(19) United States
(12) Patent Application Publication
(10) Pub. No.: US 2024/0132591 A1
(43) Pub. Date: Apr. 25, 2024

(54) PANCREAS TARGETED THERAPEUTICS AND USES THEREOF

(71) Applicant: PANDION OPERATIONS, INC., Watertown, MA (US)

(72) Inventors: Daniel RIOS, Boston, MA (US); Kevin OTIPOBY, Ashland, MA (US); Joanne L. VINEY, Belmont, MA (US); Nathan HIGGINSON-SCOTT, Hingham, MA (US)

(73) Assignee: PANDION OPERATIONS, INC., Watertown, MA (US)

(21) Appl. No.: 18/264,940

(22) PCT Filed: Feb. 22, 2022
(86) PCT No.: PCT/US2022/070765

§ 371 Date: Aug. 9, 2023

Related U.S. Application Data
(60) Provisional application No. 63/152,030, filed on Feb. 22, 2021.

(51) Int. Cl. C07K 16/28; A61P 3/10

(52) U.S. Cl. CPC C07K 16/2818; A61P 3/10; C07K 16/2803; A61K 2039/505; C07K 2317/31; C07K 2317/622; C07K 2317/75

(57) ABSTRACT

The disclosure relates to methods and compositions for treating Type 1 diabetes, such as compositions comprising an effector domain linked to a protein that binds to MAdCAM.

### CROSS-REFERENCE TO RELATED APPLICATIONS

This application claims priority to U.S. Provisional Application No. 63/152,030, filed on Feb. 22, 2021, which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Feb. 10, 2022, is named 145256_001902_SL and is 739,761 bytes in size.

### FIELD

The embodiments provided herein relate to, for example, methods and compositions for local or targeted immune-privilege.

### BACKGROUND

Instances of unwanted immune responses, e.g., as in the rejection of transplanted tissue or in autoimmune disorders, constitute a major health problem for millions of people across the world. Long-term outcomes for organ transplantation are frequently characterized by chronic rejection, and eventual failure of the transplanted organ. More than twenty autoimmune disorders are known, affecting essentially every organ of the body, and affecting over fifty million people in North America alone. The broadly active immunosuppressive medications used to combat the pathogenic immune response in both scenarios have serious side effects. Programmed cell death protein 1 (PD-1) is an inhibitory immune checkpoint molecule present on the surface of T cells, and others. PD-1 binds to two ligands, PD-L1 and PD-L2, minimizing or preventing activation and function of said T cells. PD-1 targeted therapies have emerged as ways of providing local or targeted immune privilege. The present disclosure provides for methods and compounds that provide local or targeted immune privilege.

### SUMMARY

The present disclosure provides for methods of providing local immune privilege. In some embodiments, methods of treating or preventing Type 1 diabetes comprising administering to a subject in need thereof, an anti-PD-1 agonist antibody linked to an anti-MAdCAM antibody, or antigen binding fragment thereof, are provided. In some embodiments, the anti-MAdCAM antibody, or antigen binding fragment thereof, comprises:
i) a heavy chain variable region comprising heavy chain CDR1, CDR2, and CDR3 sequences, wherein the heavy chain CDR1 sequence has an amino acid sequence having at least one amino acid substitution relative to SEQ ID NO: 1499; the heavy chain CDR2 has an amino acid sequence having at least one amino acid substitution relative to SEQ ID NO: 1506; and the heavy chain CDR3 has an amino acid sequence having at least one amino acid substitution relative to SEQ ID NOs: 1507, 1531, or 1532; and
(ii) a light chain variable region comprising light chain CDR1, CDR2, and CDR3 sequences, wherein the light chain CDR1 sequence has an amino acid sequence having at least one amino acid substitution relative to SEQ ID NO: 1502; the light chain CDR2 has an amino acid sequence having at least one amino acid substitution relative to SEQ ID NO: 1497; and the light chain CDR3 has an amino acid sequence having at least one amino acid substitution relative to SEQ ID NO: 1498.

In some embodiments, methods of treating Type 1 diabetes comprising administering to a subject in need thereof, an effector molecule linked to an antibody, or antigen binding fragment thereof, are provided. In some embodiments, the antibody comprises:
a light chain variable region comprising an amino acid sequence having at least 90% identity to an amino acid sequence selected from SEQ ID NOs: 592, 1360, 1361, 1362, 1363, 1364, 1365, 1366, 1367, 1368, 1369, 1370, 1371, 1372, 1373, 1374, 1375, 1376, 1380, 1381, 1382, 1383, 1384, 1385, 1386, 1464, 1465, 1466, 1467, or 1543; and
a heavy chain variable region comprising an amino acid sequence having at least 90% identity to an amino acid sequence selected from SEQ ID NOs: 591, 1346, 1347, 1348, 1349, 1350, 1351, 1352, 1353, 1354, 1355, 1356, 1357, 1358, 1377, 1378, 1379, 1387, 1388, 1389, 1390, 1391, 1392, 1393, 1394, 1395, 1396, 1397, 1398, 1439, 1440, 1441, 1442, 1443, 1444, 1445, 1450, 1451, 1452, 1453, 1454, 1455, 1456, 1457, 1458, 1459, 1460, 1461, 1462, 1463, 1469, 1470, 1471, 1472, 1473, 1474, 1475, 1477, 1480, 1542, 1544, or 1545.

In some embodiments, methods of delaying, reducing, treating, or preventing hyperglycemia comprising administering, to a subject in need thereof, a composition comprising an effector molecule linked to an anti-MAdCAM antibody, or antigen binding fragment thereof; and a pharmaceutically acceptable carrier, are provided.

In some embodiments, methods of treating Type 1 diabetes comprising administering to a subject in need thereof, a composition comprising an effector molecule linked to an antibody, or antigen binding fragment thereof, are provided. In some embodiments, the composition comprises:
a) the antibody is in an scFv orientation and comprises:
   i) a heavy chain variable region comprising heavy chain CDR1, CDR2, and CDR3 sequences, wherein the heavy chain CDR1 sequence has an amino acid sequence having one or zero amino acid substitution relative to SEQ ID NO: 1499; the heavy chain CDR2 has an amino acid sequence having one or zero amino acid substitution relative to SEQ ID NO: 1506; and the heavy chain CDR3 has an amino acid sequence having one or zero amino acid substitution relative to SEQ ID NOs: 1507, 1531, or 1532; and
   (ii) a light chain variable region comprising light chain CDR1, CDR2, and CDR3 sequences, wherein the light chain CDR1 sequence has an amino acid sequence having one or zero amino acid substitution relative to SEQ ID NO: 1502; the light chain CDR2 has an amino acid sequence having one or zero amino acid substitution relative to SEQ ID NO: 1497; and the light chain CDR3 has an amino acid sequence having one or zero amino acid substitution relative to SEQ ID NO: 1498; and
b) the effector molecule is in a Fab orientation and comprises:
   i) a heavy chain variable region comprising heavy chain CDR1, CDR2, and CDR3 sequences, wherein the heavy chain CDR1 sequence has an amino acid sequence having one or zero amino acid substitution relative to SEQ ID NO: 1481, or 1487; the heavy chain CDR2 has an amino acid sequence having one or zero amino acid substitution relative to SEQ ID NO: 1482, or 1488; and the heavy chain CDR3 has an amino acid sequence having one or zero amino acid substitution relative to SEQ ID NOs: 1483, or 1489; and
   (ii) a light chain variable region comprising light chain CDR1, CDR2, and CDR3 sequences, wherein the light chain CDR1 sequence has an amino acid sequence having one or zero amino acid substitution relative to SEQ ID NO: 1484, 1490, or 1214; the light chain CDR2 has an amino acid sequence having one or zero amino acid substitution relative to SEQ ID NO: 1485, or 1491; and the light chain CDR3 has an amino acid sequence having one or zero amino acid substitution relative to SEQ ID NO: 1486, or 1492.

In some embodiments, methods of treating Type 1 diabetes comprising administering to a subject in need thereof, an effector molecule linked to an antibody, or antigen binding fragment thereof, are provided, wherein:
a) the antibody is in an scFv orientation and comprises:
a light chain variable region comprising an amino acid sequence having at least 95% identity to an amino acid sequence selected from SEQ ID NOs: 1367; and
a heavy chain variable region comprising an amino acid sequence having at least 95% identity to an amino acid sequence selected from SEQ ID NOs: 1445; and
b) the effector molecule is in a Fab orientation and comprises:
a light chain variable region comprising an amino acid sequence having at least 95% identity to an amino acid sequence selected from SEQ ID NOs: 1359, 1449, or 1479; and
a heavy chain variable region comprising an amino acid sequence having at least 95% identity to an amino acid sequence selected from SEQ ID NOs: 1438, 1446, 1447, 1448, 1476, or 1478.

### BRIEF DESCRIPTION OF THE DRAWINGS

FIG. 1 depicts a bi-specific therapeutic compund in a tandem scFv-Fc format containing a targeting scFv domain and an effector domain consisting of either an scFv or a sequence corresponding to the endogenous ligand. The depiction is an example of a non-limiting embodiment of the therapeutic compounds provided herein.

FIG. 2 depicts a T cell bound to therapeutic componds disclosed herein. In state 1 the effector domain of a bi-specific binds inhibitory receptors of T cells while in systemic circulation, with neither agonism or antagonism of the receptor occurring. In state 2, the targeting domain of bi-specific binds to target organ leading to bi-specific multimerization on the target organ surface. During T cell recognition of target organ, multimerized effector domains bind, cluster and signal through T cell inhibitory molecules. The depiction is an example of a non-limiting illustration of how a therapeutic compound provided herein could function.

FIG. 3A depicts a non-limiting illustration of the therapeutic compounds provided herein.

FIG. 3B depicts a non-limiting illustration of the therapeutic compounds provided herein.

FIG. 4 depicts a non-limiting illustration of the therapeutic compounds provided herein.

FIG. 5 depicts a non-limiting illustration of the therapeutic compounds provided herein.

FIG. 6 depicts a non-limiting illustration of the therapeutic compounds provided herein.

FIG. 7 depicts a non-limiting illustration of the therapeutic compounds provided herein.

FIG. 8 depicts a non-limiting illustration of the therapeutic compounds provided herein.

FIG. 9 depicts a non-limiting illustration of the therapeutic compounds provided herein.

FIG. 10 depicts a non-limiting illustration of the therapeutic compounds provided herein.

FIG. 11 depicts a non-limiting illustration of the therapeutic compounds provided herein.

FIG. 12 depicts a non-limiting illustration of the therapeutic compounds provided herein.

FIG. 13 depicts a non-limiting illustration of the therapeutic compounds provided herein.

FIG. 14 depicts a non-limiting illustration of the therapeutic compounds provided herein.

FIG. 15 depicts a non-limiting illustration of the therapeutic compounds provided herein.

FIG. 16 depicts a non-limiting illustration of the therapeutic compounds provided herein.

FIG. 17 depicts a non-limiting illustration of the therapeutic compounds provided herein.

FIG. 18 depicts a non-limiting illustration of the therapeutic compounds provided herein.

FIG. 19 depicts a non-limiting illustration of the therapeutic compounds provided herein.

FIGS. 20A and 20B depict localization of PD-1-MAdCAM antibodies in the gut 4 weeks following subcutaneous administration of said antibodies.

FIG. 21A depicts localization of an anti-PD-1-MAdCAM bispecific molecule in mesenteric lymph node.

FIG. 21B depicts localization of the anti-PD-1-MAdCAM bispecific molecule in pancreatic lymph node.

FIG. 21C depicts localization of the anti-PD-1-MAdCAM bispecific molecule in pancreas of NOD mice.

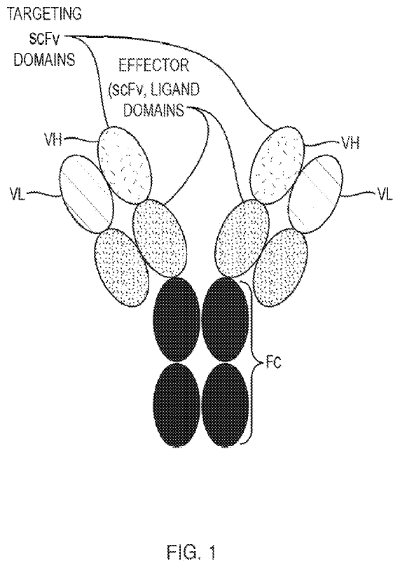

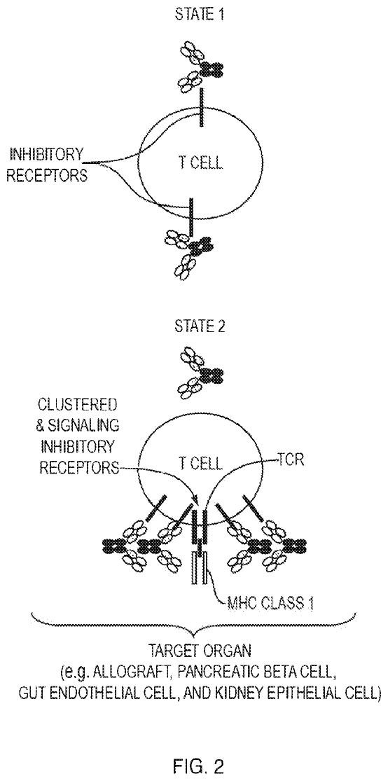

### DETAILED DESCRIPTION

This application incorporates by reference each of the following in its entirety: U.S. Provisional Application No. 63/115,243 filed Nov. 18, 2020, U.S. Provisional Application No. 63/115,235 filed Nov. 18, 2020, PCT Application No. PCT/US2020/046920 filed Aug. 19, 2020, U.S. Non-Provisional application Ser. No. 16/997,238 filed Aug. 19, 2020, PCT Application No. PCT/US2020/033707 filed May 20, 2020, and U.S. Provisional Application No. 62/850,172, filed May 20, 2019, U.S. application Ser. No. 15/922,592 filed Mar. 15, 2018 and PCT Application No. PCT/US2018/022675, filed Mar. 15, 2018. This application also incorporate by reference, each of the following in their entirety: U.S. Provisional Application No. 62/721,644, filed Aug. 23, 2018, U.S. provisional Application No. 62/675,972 filed May 24, 2018, U.S. provisional Application No. 62/595,357 filed Dec. 6, 2017, U.S. Provisional Application No. 62/595,348, filed Dec. 6, 2017, U.S. Non-Provisional application Ser. No. 16/109,875, filed Aug. 23, 2018, U.S. Non-Provisional application Ser. No. 16/109,897, filed Aug. 23, 2018, U.S. Non-Provisional application Ser. No. 15/988,311, filed May 24, 2018, PCT Application No. PCT/US2018/034334, filed May 24, 2018, and, PCT/US2018/062780, filed Nov. 28, 2018, each of which is hereby incorporated by reference in their entirety.

As used herein and in the appended claims, the singular forms “a”, “an” and “the” include plural reference unless the context clearly dictates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by +5% and remain within the scope of the disclosed embodiments. Thus, about 100 means 95 to 105.

As used herein, the term “animal” includes, but is not limited to, humans and non-human vertebrates such as wild, domestic, and farm animals. As used herein, the term “mammal” means a rodent (i.e., a mouse, a rat, or a guinea pig), a monkey, a cat, a dog, a cow, a horse, a pig, or a human. In some embodiments, the mammal is a human.

As used herein, the term “contacting” means bringing together of two elements in an in vitro system or an in vivo system. For example, “contacting” a therapeutic compound with an individual or patient or cell includes the administration of the compound to an individual or patient, such as a human, as well as, for example, introducing a compound into a sample containing a cellular or purified preparation containing target.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps. Any composition or method that recites the term “comprising” should also be understood to also describe such compositions as consisting, consisting of, or consisting essentially of the recited components or elements.

As used herein, the term “fused” or “linked” when used in reference to a protein having different domains or heterologous sequences means that the protein domains are part of the same peptide chain that are connected to one another with either peptide bonds or other covalent bonding. The domains or section can be linked or fused directly to one another or another domain or peptide sequence can be between the two domains or sequences and such sequences would still be considered to be fused or linked to one another. In some embodiments, the various domains or proteins provided for herein are linked or fused directly to one another or a linker sequences, such as the glycine/serine sequences described herein link the two domains together. Two peptide sequences are linked directly if they are directly connected to one another or indirectly if there is a linker or other structure that links the two regions. A linker can be directly linked to two different peptide sequences or domains.

As used herein, the term “individual,” “subject,” or “patient,” used interchangeably, means any animal, including mammals, such as mice, rats, other rodents, rabbits, dogs, cats, swine, cattle, sheep, horses, or primates, such as humans.

As used herein, the term “inhibit” refers to a result, symptom, or activity being reduced as compared to the activity or result in the absence of the compound that is inhibiting the result, symptom, or activity. In some embodiments, the result, symptom, or activity, is inhibited by about, or, at least, 10%, 20%, 30%, 40%, 50%, 60%, 70%, 80%, 90%, 95%, or 99%. A result, symptom, or activity can also be inhibited if it is completely elimination or extinguished.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, or condition is prevalent. In some embodiments, the subject is at risk of developing a particular disease or disorder that a treatment is intended to treat and/or prevent. Those “in need of treatment” include those patients that may benefit form treatment with the methods of the inventions, e.g. a patient suffering from or at risk of developing an autoimmune disorder or diabetes.

As used herein, the phrase “integer from X to Y” means any integer that includes the endpoints. For example, the phrase “integer from 1 to 5” means 1, 2, 3, 4, or 5.

In some embodiments, therapeutic compounds are provided herein. In some embodiments, the therapeutic compound is a protein or a polypeptide, that has multiple peptide chains that interact with one another. The polypeptides can interact with one another through non-covalent interactions or covalent interactions, such as through disulfide bonds or other covalent bonds. Therefore, if an embodiment refers to a therapeutic compound it can also be said to refer to a protein or polypeptide as provided for herein and vice versa as the context dictates.

As used herein, the phrase “ophthalmically acceptable” means having no persistent detrimental effect on the treated eye or the functioning thereof, or on the general health of the subject being treated. However, it will be recognized that transient effects such as minor irritation or a “stinging” sensation are common with topical ophthalmic administration of drugs and the existence of such transient effects is not inconsistent with the composition, formulation, or ingredient (e.g., excipient) in question being “ophthalmically acceptable” as herein defined. In some embodiments, the pharmaceutical compositions can be ophthalmically acceptable or suitable for ophthalmic administration.

“Specific binding” or “specifically binds to” or is “specific for” a particular antigen, target, or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen, target, or an epitope can be exhibited, for example, by an antibody having a K_{D} for an antigen or epitope of at least about 10^{-4M}, at least about 10^{-5M}, at least about 10^{-6M}, at least about 10^{-7M}, at least about 10^{-8M}, at least about 10^{-9M} alternatively at least about 10^{-10M} at least about 10^{-11M} at least about 10^{-12M}, or greater, where K_{D} refers to a dissociation rate of a particular antibody-target interaction. Typically, an antibody that specifically binds an antigen or target will have a K_{D} that is, or at least, 2-, 4-, 5-, 10-, 20-, 50-, 100-, 500-, 1000-, 5,000-, 10,000-, or more times greater for a control molecule relative to the antigen or epitope.

In some embodiments, specific binding for a particular antigen, target, or an epitope can be exhibited, for example, by an antibody having a K_{A} or Kₐ for a target, antigen, or epitope of at least 2-, 4-, 5-, 20-, 50-, 100-, 500-, 1000-, 5,000-, 10,000- or more times greater for the target, antigen, or epitope relative to a control, where K_{A} or Kₐ refers to an association rate of a particular antibody-antigen interaction.

As provided herein, the therapeutic compounds and compositions can be used in methods of treatment as provided herein. As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. For purposes of these embodiments, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of an auto-immune disease/disorder” means an activity that alleviates or ameliorates any of the primary phenomena or secondary symptoms associated with the auto-immune disease/disorder or other condition described herein. Methods for the treatment of various diseases or conditions are provided herein. The therapeutic treatment can also be administered prophylactically to preventing or reduce the disease or condition before the onset.

### PD-1 Agonists

Provided herein are therapeutic compounds, e.g., therapeutic protein molecules, e.g., fusion proteins, including a targeting moiety and an effector binding/modulating moiety, typically as separate domains. Also provided are methods of using and making the therapeutic compounds. The targeting moiety serves to localize the therapeutic compound, and thus the effector binding/modulating moiety, to a site at which immune-privilege is desired. As used herein, “immune privilege” means lack of, or suppression of an inflammatory response. As a non-limiting example, immune privilege includes situations where a tissue or site in the body is able to tolerate the introduction of antigens without eliciting an inflammatory immune response (Forester J. V., Lambe H. Xu, Cornall R. Immune Privilege or privileged immunity? Mucosal Immunology, 1, 372-381 (2008)).

The effector binding/modulating moiety comprises one or more of: (a) an immune cell inhibitory molecule binding/modulating moiety (an ICIM binding/modulating moiety): (b) an immunosuppressive immune cell binding/modulating moiety (an IIC binding/modulating moiety); (c) a soluble molecule binding/modulating moiety (a SM binding/modulating moiety) or (d) a molecule that blocks or inhibits immune cell stimulatory molecule binding/modulating moiety (referred to herein as an ICSM binding/modulating moiety). In some embodiments, the ICSM inhibits immune activation by, for example, blocking the interaction between a costimulatory molecule and its counterstructure. In some embodiments, a therapeutic compound comprises: (a) and (b); (a) and (c); (a) and (d); (b) and (c); (b) and (d); (c) and (d); or (a), (b), (c), and (d).

The present disclosure provides, for example, molecules that can act as PD-1 agonists. Without being bound to any particular theory, agonism of PD-1 inhibits T cell activation/signaling and can be accomplished by different mechanisms. For example crosslinking of bead-bound functional PD-1 agonists can lead to agonism. Functional PD-1 agonists have been described (Akkaya. Ph.D. Thesis: Modulation of the PD-1 pathway by inhibitory antibody superagonists. Christ Church College, Oxford, UK, 2012), which is hereby incorporated by reference. Crosslinking of PD-1 with two mAbs that bind non-overlapping epitopes induces PD-1 signaling (Davis, US 2011/0171220), which is hereby incorporated by reference. Another example is illustrated through the use of a goat anti-PD-1 antiserum (e.g. AF1086, R&D Systems), which acts as an agonist when soluble (Said et al., 2010, Nat Med. 2010 April; 16(4):452-9) which is hereby incorporated by reference. Non-limiting examples of PD-1 agonists that can be used in the present embodiments include, but are not limited to, UCB clone 19 or clone 10, PD1AB-1, PD1AB-2, PD1AB-3, PD1AB-4 and PD1AB-5, PD1AB-6 (Anaptys/Celgene), PD1-17, PD1-28, PD1-33 and PD1-35 (Collins et al, US 2008/0311117 A1 Antibodies against PD-1 and uses therefor, which is incorporated by reference), or can be a bi-specific, monovalent anti-PD-1/anti-CD3 (Ono), and the like. In some embodiments, the PD-1 agonist antibodies can be antibodies that block binding of PD-L1 to PD-1. In some embodiments, the PD-1 agonist antibodies can be antibodies that do not block binding of PD-L1 to PD-1.

PD-1 agonism can be measured by any method, such as the methods described in the examples. For example, cells can be constructed that express, including stably express, constructs that include a human PD-1 polypeptide fused to a β-galactosidase “Enzyme donor” and 2) a SHP-2 polypeptide fused to a β-galactosidase “Enzyme acceptor.” Without being bound by any theory, when PD-1 is engaged, SHP-2 is recruited to PD-1. The enzyme acceptor and enzyme donor form a fully active b-galactosidase enzyme that can be assayed. Although, the assay does not directly show PD-1 agonism, but shows activation of PD-1 signaling. PD-1 agonism can also be measured by measuring inhibition of T cell activation because, without being bound to any theory, PD-1 agonism inhibits anti-CD3-induced T cell activation. For example, PD-1 agonism can be measured by preactivating T cells with PHA (for human T cells) or ConA (for mouse T cells) so that they express PD-1. The cells can then be reactivated with anti-CD3 in the presence of anti-PD-1 (or PD-L1) for the PD-1 agonism assay. T cells that receive a PD-1 agonist signal in the presence of anti-CD3 will show decreased activation, relative to anti-CD3 stimulation alone. Activation can be readout by proliferation or cytokine production (IL-2, IFNg, IL-17) or other markers, such as CD69 activation marker. Thus, PD-1 agonism can be measured by either cytokine production or cell proliferation. Other methods can also be used to measure PD-1 agonism.

PD-1 is an Ig superfamily member expressed on activated T cells and other immune cells. The natural ligands for PD-1 appear to be PD-L1 and PD-L2. Without being bound to any particular theory, when PD-L1 or PD-L2 bind to PD-1 on an activated T cell, an inhibitory signaling cascade is initiated, resulting in attenuation of the activated T effector cell function. Thus, blocking the interaction between PD-1 on a T cell, and PD-L1/2 on another cell (for example, a tumor cell) with a PD-1 antagonist is known as checkpoint inhibition, and releases the T cells from inhibition. In contrast, PD-1 agonist antibodies can bind to PD-1 and send an inhibitory signal and attenuate the function of a T cell. Thus, PD-1 agonist antibodies can be incorporated into various embodiments described herein as an effector molecule binding/modulating moiety (sometimes also referred to herein as an effector molecule), which can accomplish localized tissue-specific immunomodulation when paired with a targeting moiety.

The effector molecule binding/modulating moiety can provide an immunosuppressive signal or environment in a variety of ways. In some embodiments, the effector binding/modulating moiety comprises an ICIM binding/modulating moiety that directly binds and (under the appropriate conditions as described herein) activates an inhibitory receptor expressed by immune cells responsible for driving disease pathology. In another embodiment the effector binding/modulating moiety comprises and IIC binding/modulating moiety and binds and accumulates immunosuppressive immune cells. In some embodiments, the accumulated immune suppressive cells promote immune privilege. In another embodiment the effector binding/modulating moiety comprises an SM binding/modulating moiety which manipulates the surrounding microenvironment to make it less permissible for the function of immune cells, e.g., immune cells driving disease pathology. In some embodiments, the SM binding/modulating moiety depletes an entity that promotes immune attack or activation. In some embodiments the effector binding/modulating moiety comprises an ICSM binding/modulating moiety that binds a member of a pair of stimulatory molecules, e.g., costimulatory molecules, and inhibits the interaction between the costimulatory molecule and the costimulatory molecule counterstructure, such as, but not limited to, OX40 or CD30 or CD40 and OX40L, or CD30L or CD40L and inhibits the immune stimulation of a cell, such as, but not limited to, a T cell, B cell, NK cell, or other immune cell comprising a member of the pair.

The targeting moiety and effector binding/modulating moiety are physically tethered, covalently or non-covalently, directly or through a linker entity, to one another, e.g., as a member of the same protein molecule in a therapeutic protein molecule. In some embodiments, the targeting and effector moieties are provided in a therapeutic protein molecule, e.g., a fusion protein, typically as separate domains. In some embodiments, the targeting moiety, the effector binding/modulating moiety, or both each comprises a single domain antibody molecule, e.g., a camelid antibody VHH molecule or human soluble VH domain. It may also contain a single-chain fragment variable (scFv) or a Fab domain. In some embodiments, the therapeutic protein molecule, or a nucleic acid, e.g., an mRNA or DNA, encoding the therapeutic protein molecule, can be administered to a subject. In some embodiments, the targeting and effector molecule binding/modulating moieties are linked to a third entity, e.g., a carrier, e.g., a polymeric carrier, a dendrimer, or a particle, e.g., a nanoparticle. The therapeutic compounds can be used to down regulate an immune response at or in a tissue at a selected target or site while having no or substantially less immunosuppressive function systemically. The target or site can comprise donor tissue or autologous tissue.

Provided herein are methods of providing site-specific immune privilege for a transplanted donor tissue, e.g., an allograft tissue, e.g., a tissue described herein, e.g., an allograft liver, an allograft kidney, an allograft heart, an allograft pancreas, an allograft thymus or thymic tissue, allograft skin, or an allograft lung, with therapeutic compounds disclosed herein. In embodiments the treatment minimizes rejection of, minimizes immune effector cell mediated damage to, prolongs acceptance of, or prolongs the functional life of, donor transplant tissue.

Also provided herein are methods of inhibiting graft versus host disease (GVHD) by minimizing the ability of donor immune cells, e.g., donor T cells, to mediate immune attack of recipient tissue, with therapeutic compounds disclosed herein.

Also provided herein are methods of treating, e.g., therapeutically treating or prophylactically treating (or preventing), an auto-immune disorder or autoimmune response in a subject by administration of a therapeutic compound disclosed herein, e.g., to provide site or tissue specific modulation of the immune system. In some embodiments, the method provides tolerance to, minimization of the rejection of, minimization of immune effector cell mediated damage to, or prolonging a function of, subject tissue. In some embodiments, the therapeutic compound includes a targeting moiety that targets, e.g., specifically targets, the tissue under, or at risk for, autoimmune attack. Non-limiting exemplary tissues include, but are not limited to, the pancreas, myelin, salivary glands, synoviocytes, and myocytes.

In some embodiments, administration of the therapeutic compound begins after the disorder is apparent. In some embodiments, administration of the therapeutic compound, begins prior to onset, or full onset, of the disorder. In some embodiments, administration of the therapeutic compound, begins prior to onset, or full onset, of the disorder, e.g., in a subject having the disorder, a high-risk subject, a subject having a biomarker for risk or presence of the disorder, a subject having a family history of the disorder, or other indicator of risk of, or asymptomatic presence of, the disorder. For example, in some embodiments, a subject having islet cell damage but which is not yet diabetic, is treated.

While not wishing to be bound by theory, it is believed that the targeting moiety functions to bind and accumulate the therapeutic compound to a target selectively expressed at the anatomical site where immune privilege is desired. In some embodiments, e.g., in the context of donor tissue transplantation, the target moiety binds to a target, e.g., an allelic product, present in the donor tissue but not the recipient. For treatment of autoimmune disorders, the targeting moiety binds a target preferentially expressed at the anatomical site where immune privilege is desired, e.g., in the pancreas. In some embodiments, the polypeptide or antibody of the disclosure binds to a pancreatic cell. In some embodiments, the pancreatic cell is a pancreatic endothelial cell. For treatment of GVHD, the targeting moiety targets the host tissue, and protects the host against attack from transplanted immune effector cells derived from transplanted tissue.

Again, while not wishing to be bound by theory it is believed that the effector binding/modulating moiety serves to deliver an immunosuppressive signal or otherwise create an immune privileged environment.

Effector, as that term is used herein, refers to an entity, e.g., a cell or molecule, e.g., a soluble or cell surface molecule, which mediates an immune response.

Effector ligand binding molecule, as used herein, refers to a polypeptide that has sufficient sequence from a naturally occurring counter-ligand of an effector, that it can bind the effector with sufficient specificity that it can serve as an effector binding/modulating molecule. In some embodiments, it binds to effector with at least 10, 20, 30, 40, 50, 60, 70, 80, 90, or 95% of the affinity of the naturally occurring counter-ligand. In some embodiments, it has at least 60, 70, 80, 90, 95, 99, or 100% sequence identity, or substantial sequence identity, with a naturally occurring counter-ligand for the effector.

Effector specific binding polypeptide, as used herein, refers to a polypeptide that can bind with sufficient specificity that it can serve as an effector binding/modulating moiety. In some embodiments, a specific binding polypeptide comprises an effector ligand binding molecule.

Unless otherwise defined, all technical and scientific terms used herein have the same meaning as commonly understood by one of ordinary skill in the art to which these embodiments belong. Although methods and materials similar or equivalent to those described herein can be used in the practice or testing of the present embodiments, suitable methods and materials are described below. All publications, patent applications, patents, and other references mentioned herein are incorporated by reference in their entirety. In addition, the materials, methods, and examples are illustrative only and not intended to be limiting. Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the embodiments will be apparent from the description and drawings, and from the claims.

### Additional Definitions

Unless defined otherwise, all technical and scientific terms used herein have the same meaning as commonly understood by one of ordinary skill in the art to which the embodiments pertains. In describing and claiming the present embodiments, the following terminology and terminology otherwise referenced throughout the present application will be used according to how it is defined, where a definition is provided.

It is also to be understood that the terminology used herein is for the purpose of describing particular embodiments only, and is not intended to be limiting.

Antibody molecule, as that term is used herein, refers to a polypeptide, e.g., an immunoglobulin chain or fragment thereof, comprising at least one functional immunoglobulin variable domain sequence. An antibody molecule encompasses antibodies (e.g., full-length antibodies) and antibody fragments. In some embodiments, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. An antibody fragment, e.g., functional fragment, comprises a portion of an antibody, e.g., Fab, Fab′, F(ab′)2, F(ab)2, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)2 fragments, and single chain variable fragments (scFvs).

Immunoglobulin chains exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminus to C-terminus, both light and heavy chains comprise the domains FR1, CDR1, FR2, CDR2, FR3, CDR3 and FR4. The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987); Chothia et al. Nature 342:878-883 (1989). In some embodiments, the antibodies provided herein comprise the same FRs and different CDRs. In some embodiments, the antibodies provided herein comprise the same CDRs and different FRs. In some embodiments, mutations in the FR are in the heavy chain. In some embodiments, mutations in the FR are in the FR1 of the heavy chain. In some embodiments, mutations in the FR are in the FR2 of the heavy chain. In some embodiments, mutations in the FR are in the FR3 of the heavy chain. In some embodiments, mutations in the FR are in the FR4 of the heavy chain. In some embodiments, mutations in the FR are in the light chain. In some embodiments, mutations in the FR are in the FR1 of the light chain. In some embodiments, mutations in the FR are in the FR2 of the light chain. In some embodiments, mutations in the FR are in the FR3 of the light chain. In some embodiments, mutations in the FR are in the FR4 of the light chain. In some embodiments, mutations in the FR are in the heavy and light chains. In some embodiments, mutations in the FR are in any one or more of the FRs of the heavy and light chains.

The term “antibody molecule” also encompasses whole or antigen binding fragments of domain, or single domain, antibodies, which can also be referred to as “sdAb” or “VHH.” Domain antibodies comprise either VH or VL that can act as stand-alone, antibody fragments. Additionally, domain antibodies include heavy-chain-only antibodies (HCAbs). Domain antibodies also include a CH2 domain of an IgG as the base scaffold into which CDR loops are grafted. It can also be generally defined as a polypeptide or protein comprising an amino acid sequence that is comprised of four framework regions interrupted by three complementarity determining regions. This is represented as FR1-CDR1-FR2-CDR2-FR3-CDR3-FR4. sdAbs can be produced in camelids such as llamas, but can also be synthetically generated using techniques that are well known in the art. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91, which is hereby incorporated by reference). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113. Domain antibodies are also described in WO2004041862 and WO2016065323, each of which is hereby incorporated by reference. The domain antibodies can be a targeting moiety as described herein.

Antibody molecules can be monospecific (e.g., monovalent or bivalent), bispecific (e.g., bivalent, trivalent, tetravalent, pentavalent, or hexavalent), trispecific (e.g., trivalent, tetravalent, pentavalent, hexavalent), or with higher orders of specificity (e.g., tetraspecific) and/or higher orders of valency beyond hexavalency. An antibody molecule can comprise a functional fragment of a light chain variable region and a functional fragment of a heavy chain variable region, or heavy and light chains may be fused together into a single polypeptide.

Examples of formats for multispecific therapeutic compounds, e.g., bispecific antibody molecules are shown in the following non-limiting examples. Although illustrated with antibody molecules, they can be used as platforms for therapeutic molecules that include other non-antibody moieties as specific binding or effector moieties. In some embodiments, these non-limiting examples are based upon either a symmetrical or asymmetrical Fc formats.

For example, the figures illustrate non-limiting and varied symmetric homodimer approach. In some embodiments, the dimerization interface centers around human IgG1 CH2-CH3 domains, which dimerize via a contact interface spanning both CH2/CH2 and CH3/CH3. The resulting bispecific antibodies shown have a total valence comprised of four binding units with two identical binding units at the N-terminus on each side of the dimer and two identical units at the C-terminus on each side of the dimer. In each case the binding units at the N-terminus of the homo-dimer are different from those at the C-terminus of the homo-dimer. Using this type of bivalency for both an inhibitory T cell receptor at either terminus of the molecule and bivalency for a tissue tethering antigen can be achieved at either end of the molecule.

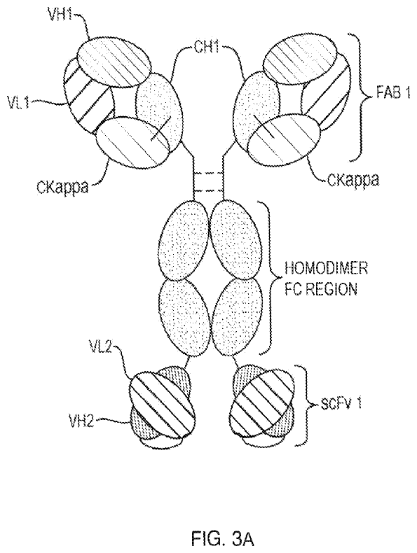

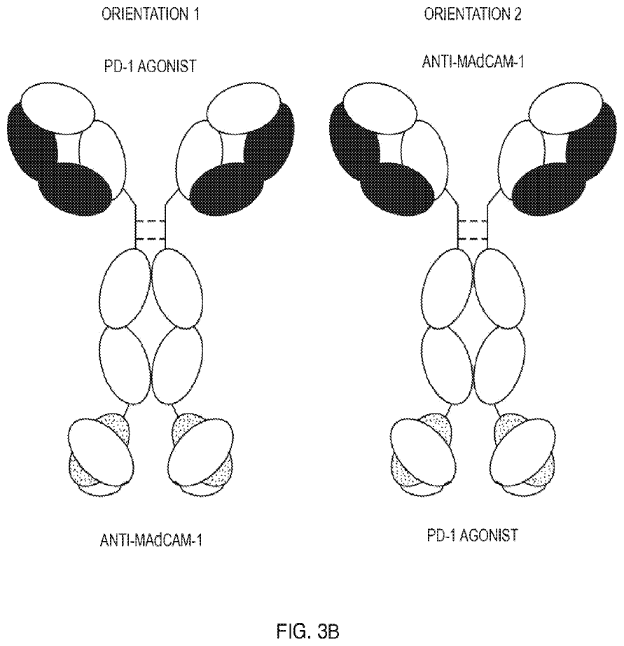

For example, in FIG. 3A, a non-limiting embodiment is illustrated. The N-terminus of the homodimer contains two identical Fab domains comprised of two identical light chains, which are separate polypeptides, interfaced with the n-terminal VH-CH1 domains of each heavy chain via the VH/VL interaction and Ckappa or Clambda interaction with CH1. The native disulfide bond between the Ckappa or Clambda with CH1 is present providing a covalent anchor between the light and heavy chains. At the c-terminus of this design are two identical scFv units where by (in this example) the c-terminus of the CH3 domain of the Fc, is followed by a flexible, hydrophilic linker typically comprised of (but not limited to) serine, glycine, alanine, and/or threonine residues, which is followed by the VH domain of each scFv unit, which is followed by a glycine/serine rich linker, followed by a VL domain. These tandem VH and VL domains associate to form a single chain fragment variable (scFv) appended at the c-terminus of the Fc. Two such units exist at the c-terminus of this molecule owing to the homodimeric nature centered at the Fc. The domain order of scFvs may be configured to be from N to C terminus either VH-Linker-VL or VL-Linker-VH.

A non-limiting example of a molecule that has different binding regions on the different ends includes a molecule that comprises, a PD-1 agonist at one end and an antibody that provides target specificity, particularly, an anti-MAdCAM-1 antibody at the other end. This can be illustrated as shown, for example, in FIG. 3B, which illustrates the molecules in different orientations.

In some embodiments, the MAdCAM antibody is a blocking or non-blocking antibody as described elsewhere herein. Without being bound to any theory, MAdCAM has been shown to interact with the headpiece of the integrin α4β7 expressed on lymphocytes via multiple residues within its two Ig superfamily I-set domains and the atomic level structural basis for that interaction has been described (Viney J L et al. (1996). J Immunol. 157, 2488-2497; Yu Y et al (2013). J Biol Chem. 288, 6284-6294; Yu Y et al (2012). J Cell Biol. 196, 131-146, each of which is incorporated by reference in its entirety). It has been shown in great structural, mechanistic and functional detail in both human (Chen J et al (2003). Nat Struct Biol. 10, 995-1001; de Chateau M et al (2001). Biochemistry. 40, 13972-13979) and mouse (Day E S et al (2002). Cell Commun Adhes. 9, 205-219; Hoshino H et al (2011). J Histochem Cytochem. 59, 572-583) molecular systems that any interaction of MAdCAM with α4β7 is dependent on three dication binding sites present in the integrin beta 7 subunit I-like domain and that these metal binding sites can coordinate with Ca2+, Mn2+, and Mg2+. Using cell adhesion assays, flow cytometry, and/or flow chamber assays in the presence of high levels of Ca2+ with or without Mg2+ or Mn2+, the MAdCAM/α4β7 interaction is shown to be of a lower functional affinity and permits rolling adhesion of lymphocytes, whereas in low Ca2+ but higher Mg2+ or Mn2+ which activates the integrin, the MAdCAM/α4β7 interaction is of a higher functional affinity and mediates firm lymphocyte adhesion (Chen J et al (2003). Nat Struct Biol. 10, 995-1001). A number of groups have shown that various cell:cell, cell:membrane prep, and/or cell:protein based adhesion/interaction assays can be utilized, with FACS, cell flow chamber based counts, or IHC based read-outs to monitor the impact of anti-MAdCAM or anti-α4β7 antibodies upon the interaction of MAdCAM with α4β7, allowing one to identify blocking or non-blocking antibodies (Nakache, M et al (1989). Nature. 337, 179-181; Streeter, P R et al (1988). Nature. 331. 41-46; Yang Y et al (1995). Scand J Immunol. 42. 235-247; Leung E et al (2004). Immunol Cell Biol. 82. 400-409; Pullen N et al (2009). B J Pharmacol. 157. 281-293; Soler D et al (2009). J Pharmacol Exp Ther. 330. 864-875; Qi J et al (2012). J Biol Chem. 287. 15749-15759). This has been exemplified in the mouse system setting with the identification of anti-mouse MAdCAM antibodies such as MECA-89 (non-blocking) and MECA-367 (blocking)) Nakache, M et al (1989). Nature. 337, 179-181; Streeter, P R et al (1988). Nature. 331. 41-46; Yang Y et al (1995). Scand J Immunol. 42. 235-247). In a human system, antibodies have been identified that block the interaction of human MAdCAM with human α4β7 such as anti-human MAdCAM PF-00547659 (Pullen N et al (2009). B J Pharmacol. 157. 281-293) and anti-human α4β7 vedolizumab (Soler D et al (2009). J Pharmacol Exp Ther. 330. 864-875), as well as antibodies that do not block the interaction such as anti-human MAdCAM clone 17F5 (Soler D et al (2009). J Pharmacol Exp Ther. 330. 864-875), and anti-human α4β7 clone J19 (Qi J et al (2012). J Biol Chem. 287. 15749-15759). Thus, the antibody can either be blocking or non-blocking based upon the desired effect. In some embodiments, the antibody is a non-blocking MAdCAM antibody. In some embodiments, the antibody is a blocking MAdCAM antibody. One non-limiting example of demonstrating whether an antibody is blocking or non-blocking can be found throughout the examples, but any method can be used. Each of the references described herein are incorporated by reference in its entirety. In some embodiments, the PD-1 Agonist is replaced with an IL-2 mutein, such as, but not limited to, the ones described herein.

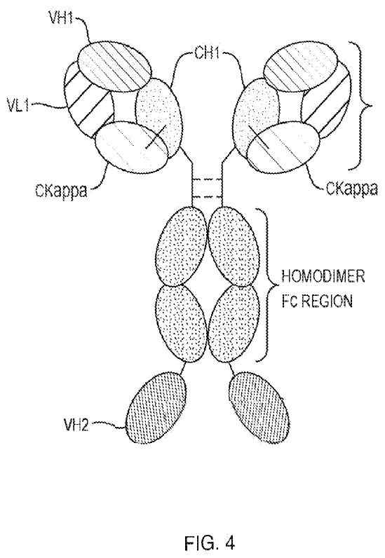

In another example, and as depicted in FIG. 4, the N-terminus of the homodimer contains two identical Fab domains comprised of two identical light chains, which are separate polypeptides, interfaced with the N-terminal VH-CH1 domains of each heavy chain via the VH/VL interaction and Ckappa or Clambda interaction with CH1. The native disulfide bond between the Ckappa or Clambda with CH1 is present providing a covalent anchor between the light and heavy chains. At the C-terminus of this design are two identical VH units (though non-antibody moieties could also be substituted here or at any of the four terminal attachment/fusion points) where by (in this example) the C-terminus of the CH3 domain of the Fc, is followed by a flexible, hydrophilic linker typically comprised of (but not limited to) serine, glycine, alanine, and/or threonine residues, which is followed by a soluble independent VH3 germline family based VH domain. Two such units exist at the C-terminus of this molecule owing to the homodimeric nature centered at the Fc.

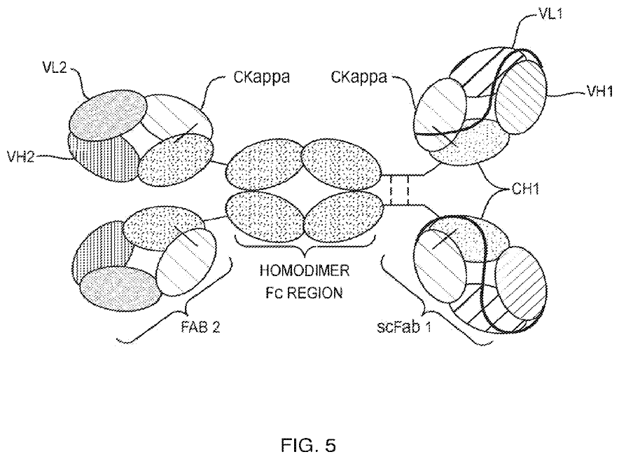

In another non-limiting example, as depicted in FIG. 5, the N-terminus of the homodimer contains two identical Fab domains comprised of two identical light chains, which, unlike FIG. 3A and FIG. 4, are physically conjoined with the heavy chain at the N-terminus via a linker between the c-terminus of Ckappa or Clambda and the N-terminus of the VH. The linker may be 36-80 amino acids in length and comprised of serine, glycine, alanine and threonine residues. The physically conjoined n-terminal light chains interface with the n-terminal VH-CH1 domains of each heavy chain via the VH/VL interaction and Ckappa or Clambda interaction with CH1. The native disulfide bond between the Ckappa or Clambda with CH1 is present providing additional stability between the light and heavy chains. At the c-terminus of this design are two identical Fab units where by (in this example) the c-terminus of the CH3 domain of the Fc, is followed by a flexible, hydrophilic linker typically comprised of (but not limited to) serine, glycine, alanine, and/or threonine residues, which is followed by a CH1 domain, followed by a VH domain at the c-terminus. The light chain that is designed to pair with the c-terminal CH1/VH domains is expressed as a separate polypeptide, unlike the N-terminal light chain which is conjoined to the n-terminal VH/CH1 domains as described. The C-terminal light chains form an interface at between VH/VL and Ckappa or Clambda with CH1. The native disulfide anchors this light chain to the heavy chain. Again, any of the antibody moieties at any of the four attachment/fusion points can be substituted with a non-antibody moiety, e.g., an effector binding/modulating moiety that does not comprise an antibody molecule.

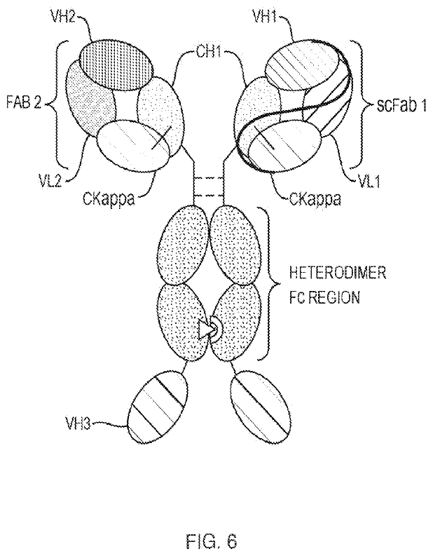

The bispecific antibodies can also be asymmetric as shown in the following non-limiting examples. Non-limiting example are also depicted in FIG. 6, FIG. 7, and FIG. 8, which illustrate an asymmetric/heterodimer approach. Again, in any of these formats, any of the antibody moieties at any of the four attachment/fusion points can be substituted with a non-antibody moiety, e.g., an effector binding/modulating moiety that does not comprise an antibody molecule. In some embodiments, the dimerization interface centers around the human IgG1 CH2-CH3 domains, which dimerize via a contact interface spanning both CH2/CH2 and CH3/CH3. However, in order to achieve heterodimerization instead of homodimerization of each heavy chain, mutations are introduced in each CH3 domain. The heterodimerizing mutations include T366W mutation (kabat) in one CH3 domain and T366S, L368A, and Y407V (kabat) mutations in the other CH3 domain. The heterodimerizing interface may be further stabilized with de novo disulfide bonds via mutation of native residues to cysteine residues such as S354 and Y349 on opposite sides of the CH3/CH3 interface. The resulting bispecific antibodies shown have a total valence comprised of four binding units. With this approach, the overall molecule can be designed to have bispecificity at just one terminus and monospecificity at the other terminus (trispecificity overall) or bispecificity at either terminus with an overall molecular specificity of 2 or 4. In the illustrative examples below, the C-terminus comprises two identical binding domains which could, for example, provide bivalent monospecificity for a tissue tethering target. At the N-terminus of all three of the illustrative examples, both binding domains comprise different recognition elements/paratopes and which could achieve recognition of two different epitopes on the same effector moiety target, or could recognize for examples a T cell inhibitory receptor and CD3. In some embodiments, the N-terminal binding moieties may be interchanged with other single polypeptide formats such as scFv, single chain Fab, tandem scFv, VH or VHH domain antibody configurations for example. Other types of recognition element may be used also, such as linear or cyclic peptides.

An example of an asymmetric molecule is depicted in FIG. 6. Referring to FIG. 6, the N-terminus of the molecule is comprised of a first light chain paired with a first heavy chain via VH/VL and Ckappa or Clambda/CH1 interactions and a covalent tether comprised of the native heavy/light chain disulfide bond. On the opposite side of this heterodimeric molecule at the N-terminus is a second light chain and a second heavy chain which are physically conjoined via a linker between the c-terminus of Ckappa or Clambda and the N-terminus of the VH. The linker may be 36-80 amino acids in length and comprised of serine, glycine, alanine and threonine residues. The physically conjoined N-terminal light chains interface with the N-terminal VH-CH1 domains of each heavy chain via the VH/VL interaction and Ckappa or Clambda interaction with CH1. The native disulfide bond between the Ckappa or Clambda with CH1 is present providing additional stability between the light and heavy chains. At the C-terminus of the molecule are two identical soluble VH3 germline family VH domains joined via an N-terminal glycine/serine/alanine/threonine based linker to the C-terminus of the CH3 domain of both heavy chain 1 and heavy chain 2.

In some embodiments, an asymmetric molecule can be as depicted in FIG. 7. For example, the N-terminus of the molecule is comprised of two different VH3 germlined based soluble VH domains linked to the human IgG1 hinge region via a glycine/serine/alanine/threonine based linker. The VH domain connected to the first heavy chain is different to the VH domain connected to the second heavy chain. At the C-terminus of each heavy chain is an additional soluble VH3 germline based VH domain, which is identical on each of the two heavy chains. The heavy chain heterodimerizes via the previously described knobs into holes mutations present at the CH3 interface of the Fc module.

In some embodiments, an asymmetric molecule can be as illustrated in FIG. 8. This example is similar to the molecule shown in FIG. 7, except both N-terminal Fab units are configured in a way that light chain 1 and light chain 2 are physically conjoined with heavy chain 1 and heavy chain 2 via a linker between the C-terminus of Ckappa or Clambda and the N-terminus of each respective VH. The linker in each case may be 36-80 amino acids in length and comprised of serine, glycine, alanine and threonine residues. The physically conjoined N-terminal light chains interface with the N-terminal VH-CH1 domains of each heavy chain via the VH/VL interaction and Ckappa or Clambda interaction with CH1. The native disulfide bond between the Ckappa or Clambda with CH1 is present providing additional stability between the light and heavy chains.

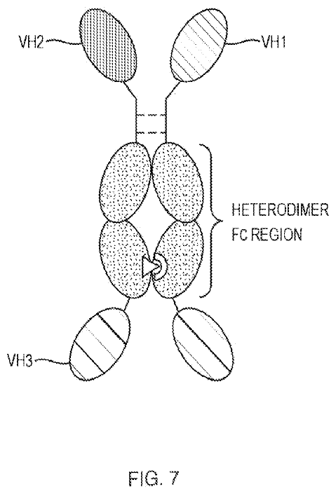

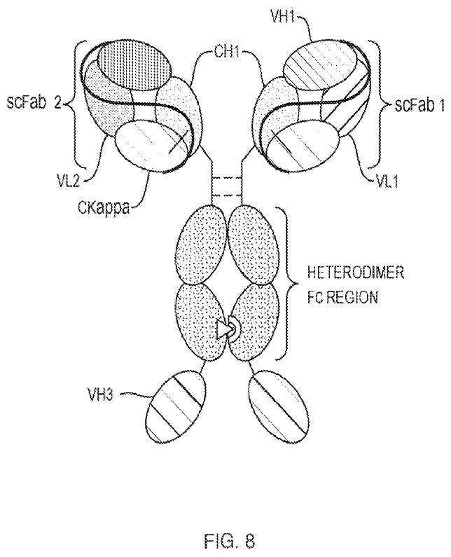

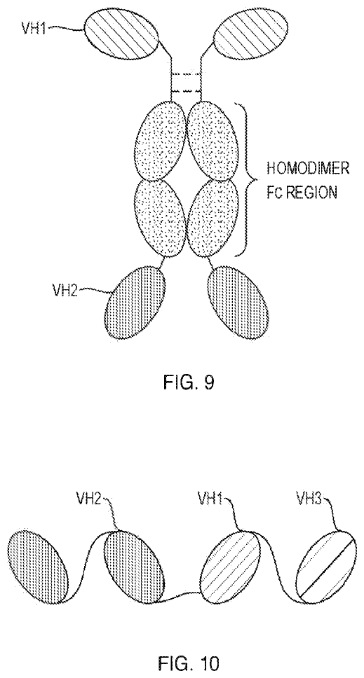

Bi-specific molecules can also have a mixed format. This is illustrated, for example, in FIG. 9, FIG. 10, and FIG. 11.

For example, FIG. 9 illustrates a homodimer Fc based approach (see FIGS. 3, 4, and 5), combined with the moiety format selection of FIG. 7, whereby the total molecular valency is four, but specificity is restricted to two specificities. The N-terminus is comprised of two identical soluble VH3 germline based VH domains and the C-terminus is comprised of two identical soluble VH3 germlined based VH domains of different specificity to the N-terminal domains. Therefore, each specificity has a valence of two. Again, in this format, any of the antibody moieties at any of the four attachment/fusion points can be substituted with a non-antibody moiety, e.g., an effector binding/modulating moiety that does not comprise an antibody molecule.

FIG. 10 illustrates another example of a bispecific molecule having a mixed format. In this example, the molecule is comprised of four VH3 germline based soluble VH domains. The first two domains have the same specificity (for example an inhibitory receptor), the 3rd domain from the N-terminus may have specificity for a tissue antigen and the fourth domain from the N-terminus may have specificity for human serum albumin (HSA), thereby granting the molecule extended half-life in the absence of an Ig Fc domain. Three glycine, serine, alanine and/or threonine rich linkers exists between domains 1 and 2, domains 2 and 3, and domains 3 and 4. This format may be configured with up to tetraspecificity, but monovalent in each case, or to have bispecificity with bivalency in each case. The order of domains can be changed. Again, in this format, any of the antibody moieties can be substituted with a non-antibody moiety, e.g., an effector binding/modulating moiety that does not comprise an antibody molecule.

FIG. 11 illustrates yet another approach. This example is similar to FIGS. 3 and 4, in that it is Fc homodimer based with two identical Fab units (bivalent monospecificity) at the N-terminus of the molecule. This example differs from FIGS. 3A and 4 in that the C-terminus of each heavy chain is appended with a tandem-scFv. Thus, in each case the C-terminus of the CH3 domain of the Fc is linked via a glycine/serine/alanine/threonine based linker to the N-terminus of a first VH domain, which is linked via the C-terminus by a 12-15 amino acid glycine/serine rich linker to the N-terminus of a first VL domain, which linked via a 25-35 amino acid glycine/serine/alanine/threonine based linker at the c-terminus to the N-terminus of a second VH domain, which is linked via the C-terminus with a 12-15 amino acid glycine/serine based linker to the N-terminus of a 2nd VL domain. In this Fc homodimer based molecule there are therefore two identical tandem scFvs at the C-terminus of the molecule offering either tetravalency for a single tissue antigen for example or bivalency to two different molecules. This format could also be adapted with a heterodimer Fc core allowing two different tandem-scFvs at the Cc-terminus of the Fc allowing for monovalent tetraspecificity at the c-terminus while retaining either bivalent monospecificity at the N-terminus or monovalent bispecificity at the N-terminal via usage of single chain Fab configurations as in FIGS. 5, 6, and 7. This molecule can therefore be configured to have 2, 3, 4, 5, or 6 specificities. The domain order of scFvs within the tandem-scFv units may be configured to be from N to C terminus either VH-Linker-VL or VL-Linker-VH. Again, in this format, any of the antibody moieties at any of the four attachment/fusion points can be substituted with a non-antibody moiety, e.g., an effector binding/modulating moiety that does not comprise an antibody molecule.

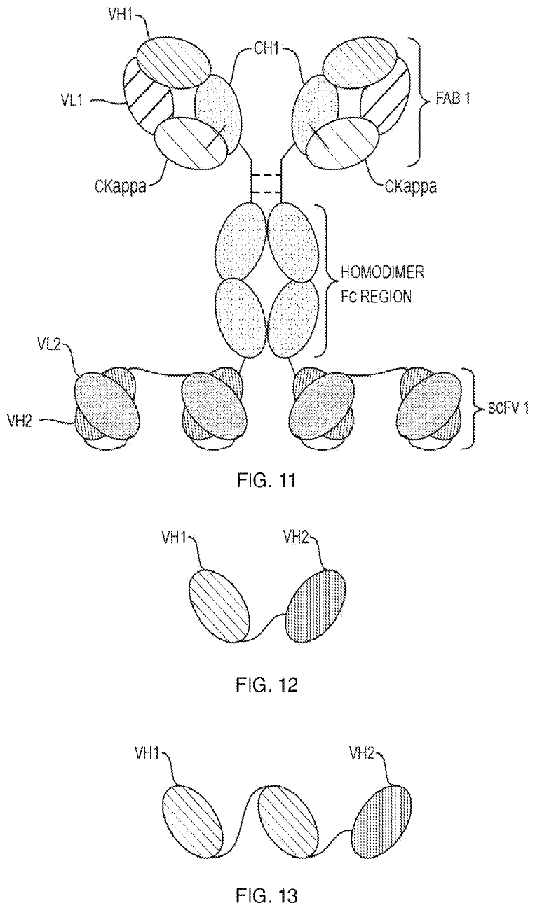

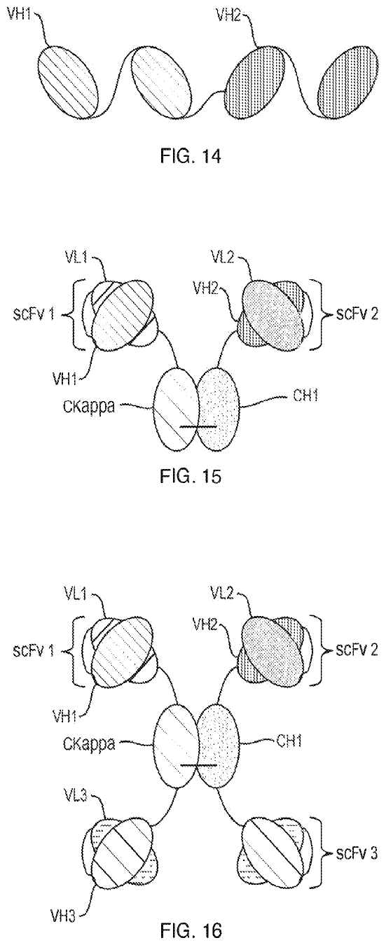

Bi-specific antibodies can also be constructed to have, for example, shorter systemic PK while having increased tissue penetration. These types of antibodies can be based upon, for example, a human VH3 based domain antibody format. These are illustrated, for example, in FIGS. 12, 13, and 14. FIGS. 12, 13, and 14 each comprised a soluble VH3 germline family based VH domain modules. Each domain is approximately 12.5 kDa allowing for a small overall MW, which, without being bound to any particular theory, should be beneficial for enhanced tissue penetration. In these examples, none of the VH domains recognize any half-life extending targets such as FcRn or HSA. As illustrated in FIG. 12, the molecule is comprised of two VH domains joined with a flexible hydrophilic glycine/serine based linker between the C-terminus of the first domain and N-terminus of the second domain. In this example one domain may recognize a T cell co-stimulatory receptor and the second may recognize a tissue tethering antigen. As illustrated in FIG. 13, the molecule is comprised of three VH domains with N-C terminal linkages of hydrophilic glycine/serine based linkers. The molecule may be configured to be trispecific but monovalent for each target. It may be bispecific with bivalency for one target and monovalency for another. As illustrated in FIG. 14, the molecule is comprised of four VH domains with N-C terminal Glycine/Serine rich linkers between each domain. This molecule may be configured to be tetraspecific, trispecific, or bispecific with varying antigenic valencies in each case. Again, in this format, any of the antibody moieties at can be substituted with a non-antibody moiety, e.g., an effector binding/modulating moiety that does not comprise an antibody molecule.

Other embodiments of bi-specific antibodies are illustrated in FIGS. 15 and 16. FIGS. 15 and 16 are comprised of the naturally heterodimerizing core of the human IgG CH1/Ckappa interface, including the c-terminal heavy/light disulfide bond which covalently anchors the interaction. This format does not contain an Fc or any moieties for half-life extension. As illustrated in FIG. 15, the molecule, at the N-terminus of the constant kappa domain is appended with an scFv fragment consisting of an N-terminal VH domain, linked at its C-terminus to the N-terminus of a VL domain via a 12-15 amino acid gly/ser based linker, which is linked by its C-terminus to the N-terminus of the constant kappa domain via the native VL-Ckappa elbow sequence. The CH1 domain is appended at the N-terminus with an scFv fragment consisting of an N-terminal VL domain linked at its c-terminus via a 12-15 amino acid gly/ser linker to the N-terminus of a VH domain, which is linked at its c-terminus to the N-terminus of the CH1 domains via the natural VH-CH1 elbow sequence. As illustrated in FIG. 16, the molecule has the same N-terminal configuration as Example 13. However the C-terminus of the constant kappa and CH1 domains are appended with scFv modules which may be in either the VH-VL or VL-VH configuration and may be either specific for the same antigen or specific for two different antigens. The VH/VL inter-domain linkers may be 12-15 amino acids in length and consisting of gly/ser residues. The scFv binding sub-units may be swapped for soluble VH domains, or peptide recognition elements, or even tandem-scFv elements. This approach can also be configured to use variable lambda and/or constant lambda domains. Again, in this format, any of the antibody moieties at any of the attachment/fusion points can be substituted with a non-antibody moiety, e.g., an effector binding/modulating moiety that does not comprise an antibody molecule.

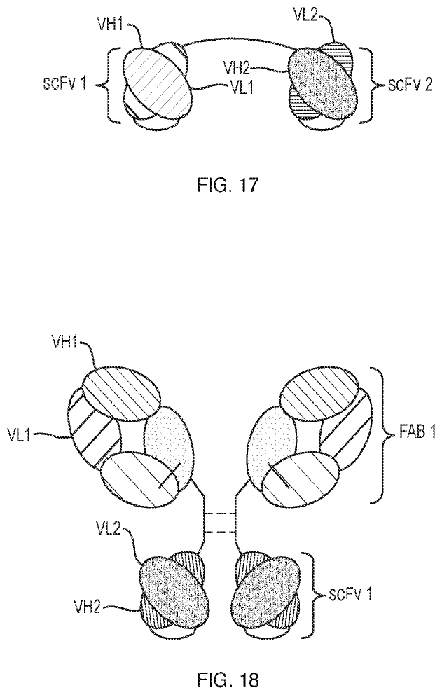

FIG. 17 illustrates another embodiment. FIG. 17 represents a tandem scFv format consisting of a first N-terminal VL domain linked at its C-terminus to the N-terminus of a first VH domain with a 12-15 amino acid gly/ser rich linker, followed at the first VH c-terminus by a 25-30 amino acid gly/ser/ala/thr based linker to the N-terminus of a second VL domain. The second VL domain is linked at the C-terminus to the N-terminus of a 2nd VH domain by a 12-15 amino acid gly/ser linker. Each scFv recognizes a different target antigen such as a co-stimulatory T cell molecule and a tissue tethering target. Again, in this format, any of the antibody moieties can be substituted with a non-antibody moiety, e.g., an effector binding/modulating moiety that does not comprise an antibody molecule.

FIG. 18 illustrates another embodiment. FIG. 18 is a F(ab′)2 scFv fusion. This consists of two identical Fab components joined via two disulfide bonds in the native human IgG1 hinge region c-terminal of the human IgG CH1 domain. The human IgG1 CH2 and CH3 domains are absent. At the c-terminus of heavy chains 1 and 2 are two identical scFv fragments linked via a gly/ser/ala/thr rich linker to the c-terminus of the huIgG1 hinge region. In the configuration shown, the VH is N-terminal in each scFv unit and linked via a 12-15 amino acid gly/ser rich linker to the N-terminus of a VL domain. An alternative configuration is N-term-VL-Linker-VH-C-term. In this design, the construct is bispecific with bivalency for reach target. Again, in this format, any of the antibody moieties at any of the four attachment/fusion points can be substituted with a non-antibody moiety, e.g., an effector binding/modulating moiety that does not comprise an antibody molecule.

Cell surface molecule binder, as that term is used herein, refers to a molecule, typically a polypeptide, that binds, e.g., specifically, to a cell surface molecule on a cell, e.g., an immunosuppressive immune cell, e.g., a Treg. In some embodiments, the cell surface binder has sufficient sequence from a naturally occurring ligand of the cell surface molecule, that it can specifically bind the cell surface molecule (a cell surface molecule ligand). In some embodiments, the cell surface binding is an antibody molecule that binds, e.g., specifically binds, the cell surface molecule.

Donor specific targeting moiety, as that term is used herein, refers to a moiety, e.g., an antibody molecule, that as a component of a therapeutic compound, localizes the therapeutic compound preferentially to an implanted donor tissue, as opposed to tissue of a recipient. As a component of a therapeutic compound, the donor specific targeting moiety provides site-specific immune privilege for a transplant tissue, e.g., an organ, from a donor.

In some embodiments, a donor specific targeting moiety it binds to the product, e.g., a polypeptide product, of an allele present at a locus, which allele is not present at the locus in the (recipient) subject. In some embodiments, a donor specific targeting moiety binds to an epitope on product, which epitope is not present in the (recipient) subject.

In some embodiments, a donor specific targeting moiety, as a component of a therapeutic compound, preferentially binds to a donor target or antigen, e.g., has a binding affinity for the donor target that is greater for donor antigen or tissue, e.g., at least 2, 4, 5, 10, 50, 100, 500, 1,000, 5,000, or 10,000 fold greater, than its affinity for than for subject antigen or tissue. In some embodiments, a donor specific targeting moiety, has a binding affinity for a product of an allele of a locus present in donor tissue (but not present in the subject) at least 2, 4, 5, 10, 50, 100, 500, 1,000, 5,000, or 10,000 fold greater, than its affinity for the product of the allele of the locus present in the subject (which allele is not present in donor tissue). Affinity of a therapeutic compound of which the donor specific moiety is a component, can be measured in a cell suspension, e.g., the affinity for suspended cells having the allele is compared with its affinity for suspended cells not having the allele. In some embodiments, the binding affinity for the donor allele cells is below 10 nM. In some embodiments, the binding affinity for the donor allele cells is below 100 pM, 50 pM, or 10 pM.

In some embodiments, the specificity for a product of a donor allele is sufficient that when the donor specific targeting moiety is coupled to an immune-down regulating effector: i) immune attack of the implanted tissue, e.g., as measured by histological inflammatory response, infiltrating T effector cells, or organ function, in the clinical setting—e.g. creatinine for the kidney, is substantially reduced, e.g., as compared to what would be seen in an otherwise similar implant but lacking the donor specific targeting moiety is coupled to an immune-down regulating effector; and/or ii) immune function in the recipient, outside or away from the implanted tissue, is substantially maintained. In some embodiments, one or more of the following is seen: at therapeutic levels of therapeutic compound, peripheral blood lymphocyte counts are not substantially impacted, e.g., the level of T cells is within 25, 50, 75, 85, 90, or 95% of normal, the level of B cells is within 25, 50, 75, 85, 90, or 95% of normal, and/or the level of granuloctyes (PMNs) cells is within 25, 50, 75, 85, 90, or 95% of normal, or the level of monocytes is within 25, 50, 75, 85, 90, or 95% of normal; at therapeutic levels of therapeutic compound, the ex vivo proliferative function of PBMCs (peripheral blood mononuclear cells) against non-disease relevant antigens is substantially normal or is within 70, 80, or 90% of normal; at therapeutic levels of therapeutic compound, the incidence or risk of risk of opportunistic infections and cancers associated with immunosuppression is not substantially increased over normal; or at therapeutic levels of therapeutic compound, the incidence or risk of risk of opportunistic infections and cancers associated with immunosuppression is substantially less than would be seen with standard of care, or non-targeted, immunosuppression. In some embodiments, the donor specific targeting moiety comprises an antibody molecule, a target specific binding polypeptide, or a target ligand binding molecule.

Elevated risk, as used herein, refers to the risk of a disorder in a subject, wherein the subject has one or more of a medical history of the disorder or a symptom of the disorder, a biomarker associated with the disorder or a symptom of the disorder, or a family history of the disorder or a symptom of the disorder.

Functional antibody molecule to an effector or inhibitory immune checkpoint molecule, as that term is used herein, refers to an antibody molecule that when present as the ICIM binding/modulating moiety of a multimerized therapeutic compound, can bind and agonize the effector or inhibitory immune checkpoint molecule. In some embodiments, the anti-effector or inhibitory immune checkpoint molecule antibody molecule, when binding as a monomer (or binding when the therapeutic compound is not multimerized), to the effector or inhibitory immune checkpoint molecule, does not antagonize, substantially antagonize, prevent binding, or prevent substantial binding, of an endogenous counter ligand of the inhibitory immune checkpoint molecule to inhibitory immune checkpoint molecule. In some embodiments, the anti-effector or inhibitory immune checkpoint molecule antibody molecule when binding as a monomer (or binding when the therapeutic compound is not multimerized), to the inhibitory immune checkpoint molecule, does not agonize or substantially agonize, the effector or inhibitory molecule.

ICIM binding/modulating moiety, as that term is used herein, refers to an effector binding/modulating moiety that, as part of a therapeutic compound, binds and agonizes a cell surface inhibitory molecule, e.g., an inhibitory immune checkpoint molecule, e.g., PD-1, or binds or modulates cell signaling, e.g., binds a FCRL, e.g., FCRL1-6, or binds and antagonizes a molecule that promotes immune function.

IIC binding/modulating moiety, as that term is used herein, refers to an effector binding/modulating moiety that, as part of a therapeutic compound, binds an immunosuppressive immune cell. In some embodiments, the IIC binding/modulating moiety increases the number or concentration of an immunosuppressive immune cell at the binding site.

ICSM binding/modulating moiety, as that term is used herein, refers to an effector binding/modulating moiety that antagonizes an immune stimulatory effect of a stimulatory, e.g., co-stimulatory, binding pair. A stimulatory or co-stimulatory binding pair, as that term is used herein, comprises two members, 1) a molecule on the surface of an immune cell; and 2) the binding partner for that cell molecule, which may be an additional immune cell, or a non-immune cell. Ordinarily, upon binding of one member to the other, assuming other requirements are met, the member on the immune cell surfaces stimulates the immune cell, e.g., a costimulatory molecule, and an immune response is promoted. In situations where the costimulatory molecule and the costimulatory molecule counterstructure are both expressed on immune cells, bi-directional activation of both cells may occur. In an embodiment an ICSM binding/modulating moiety binds and antagonizes the immune cell expressed member of a binding pair. For example, it binds and antagonizes OX40. In another embodiment, an ICSM binding/modulating moiety binds and antagonizes the member of the binding pair that itself binds the immune cell expressed member, e.g., it binds and antagonizes OX40L. In either case, inhibition of stimulation or co-stimulation of an immune cell is achieved. In an embodiment the ICSM binding/modulating moiety decreases the number or the activity of an immunostimulating immune cell at the binding site.

### Inhibitory Immune Checkpoint Molecules

An “inhibitory immune checkpoint molecule ligand molecule,” as that term is used herein, refers to a polypeptide having sufficient inhibitory immune checkpoint molecule ligand sequence, e.g., in the case of a PD-L1 molecule, sufficient PD-L1 sequence, that when present as an ICIM binding/modulating moiety of a multimerized therapeutic compound, can bind and agonize its cognate inhibitory immune checkpoint molecule, e.g., again in the case of a PD-L1 molecule, PD-1.

In some embodiments, the inhibitory immune checkpoint molecule ligand molecule, e.g., a PD-L1 molecule, when binding as a monomer (or binding when the therapeutic compound is not multimerized), to its cognate ligand, e.g., PD-1, does not antagonize or substantially antagonize, or prevent binding, or prevent substantial binding, of an endogenous inhibitory immune checkpoint molecule ligand to the inhibitory immune checkpoint molecule. E.g., in the case of a PD-L1 molecule, the PD-L1 molecule does not antagonize binding of endogenous PD-L1 to PD-1.

In some embodiments, the inhibitory immune checkpoint molecule ligand when binding as a monomer, to its cognate inhibitory immune checkpoint molecule does not agonize or substantially agonize the inhibitory immune checkpoint molecule. By way of example, e.g., a PD-L1 molecule when binding to PD-1, does not agonize or substantially agonize PD-1.

In some embodiments, an inhibitory immune checkpoint molecule ligand molecule has at least 60, 70, 80, 90, 95, 99, or 100% sequence identity, or substantial sequence identity, with a naturally occurring inhibitory immune checkpoint molecule ligand.

Exemplary inhibitory immune checkpoint molecule ligand molecules include: a PD-L1 molecule, which binds to inhibitory immune checkpoint molecule PD-1, and in embodiments has at least 60, 70, 80, 90, 95, 99, or 100% sequence identity, or substantial sequence identity, with a naturally occurring PD-L1, e.g., the PD-L1 molecule comprising the sequence of

(SEQ ID NO: 3)
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET,

or an active fragment thereof; in some embodiments, the active fragment comprises residues 19 to 290 of the PD-L1 sequence; an HLA-G molecule, which binds to any of inhibitory immune checkpoint molecules KIR2DL4, LILRB1, and LILRB2, and in embodiments has at least 60, 70, 80, 90, 95, 99, or 100% sequence identity, or substantial sequence identity, with a naturally occurring HLA-G. Exemplary HLA-G sequences include, e.g., a mature form found in the sequence at GenBank P17693.1 RecName: Full=HLA class I histocompatibility antigen, alpha chain G; AltName: Full=HLA G antigen; AltName: Full=MHC class I antigen G; Flags: Precursor, or in the sequence

(SEQ ID NO: 4)
MVVMAPRTLFLLLSGALTLTETWAGSHSMRYFSAAVSRPGRGEPRFIAMG
YVDDTQFVRFDSDSACPRMEPRAPWVEQEGPEYWEEETRNTKAHAQTDRM
NLQTLRGYYNQSEASSHTLQWMIGCDLGSDGRLLRGYEQYAYDGKDYLAL
NEDLRSWTAADTAAQISKRKCEAANVAEQRRAYLEGTCVEWLHRYLENGK
EMLQRADPPKTHVTHHPVFDYEATLRCWALGFYPAEIILTWQRDGEDQTQ
DVELVETRPAGDGTFQKWAAVVVPSGEEQRYTCHVQHEGLPEPLMLRWKQ
SSLPTIPIMGIVA.

Inhibitory molecule counter ligand molecule, as that term is used herein, refers to a polypeptide having sufficient inhibitory molecule counter ligand sequence such that when present as the ICIM binding/modulating moiety of a multimerized therapeutic compound, can bind and agonize a cognate inhibitory molecule. In some embodiments, the inhibitory molecule counter ligand molecule, when binding as a monomer (or binding when the therapeutic compound is not multimerized), to the inhibitory molecule, does not antagonize, substantially antagonize, prevent binding, or prevent substantial binding, of an endogenous counter ligand of the inhibitory molecule to the inhibitory molecule. In some embodiments, the inhibitory molecule counter ligand molecule when binding as a monomer (or binding when the therapeutic compound is not multimerized), to the inhibitory molecule, does not agonize or substantially agonize, the inhibitory molecule.

Sequence identity, percentage identity, and related terms, as those terms are used herein, refer to the relatedness of two sequences, e.g., two nucleic acid sequences or two amino acid or polypeptide sequences. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”).

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to for example any a nucleic acid sequence provided herein. BLAST protein searches can be performed with the XBLAST program, score=50, wordlength=3 to obtain amino acid sequences homologous to protein molecules provided herein. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. See http://www.ncbi.nlm.nih.gov.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified.

It is understood that the molecules and compounds of the present embodiments may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). CD39 molecule, a CD73 molecule, a Cell surface molecule binder, Donor specific targeting moiety Effector ligand binding molecule, ICIM binding/modulating moiety IIC binding/modulating moiety, an inhibitory immune checkpoint molecule ligand molecule, Inhibitory molecule counter ligand molecule, SM binding/modulating moiety, or ICSM binding/modulating moiety.

SM binding/modulating moiety, as that term is used herein, refers to an effector binding/modulating moiety that, as part of a therapeutic compound, promotes an immunosuppressive local microenvironment, e.g., by providing in the proximity of the target, a substance that inhibits or minimizes attack by the immune system of the target. In some embodiments, the SM binding/modulating moiety comprises, or binds, a molecule that inhibits or minimizes attack by the immune system of the target. In some embodiments, a therapeutic compound comprises an SM binding/modulating moiety that binds and accumulates a soluble substance, e.g., an endogenous or exogenous substance, having immunosuppressive function. In some embodiments, a therapeutic compound comprises an SM binding/modulating moiety that binds and inhibits, sequesters, degrades or otherwise neutralizes a substance, e.g., a soluble substance, typically and endogenous soluble substance, that promotes immune attack. In some embodiments, a therapeutic compound comprises an SM binding/modulating moiety that comprises an immune-suppressive substance, e.g. a fragment of protein known to be immunosuppressive. By way of example, an effector molecule binding moiety that binds, or comprises, a substance e.g., a CD39 molecule or a CD73 molecule, that depletes a component, that promotes immune effector cell function, e.g., ATP or AMP.

Specific targeting moiety, as that term is used herein, refers to donor specific targeting moiety or a tissue specific targeting moiety.

Subject, as that term is used herein, refers to a mammalian subject, e.g., a human subject. In some embodiments, the subject is a non-human mammal, e.g., a horse, dog, cat, cow, goat, or pig.

Target ligand binding molecule, as used herein, refers to a polypeptide that has sufficient sequence from a naturally occurring counter-ligand of a target ligand that it can bind the target ligand on a target tissue (e.g., donor tissue or subject target tissue) with sufficient specificity that it can serve as a specific targeting moiety. In some embodiments, it binds to target tissue or cells with at least 10, 20, 30, 40, 50, 60, 70, 80, 90, or 95% of the affinity of the naturally occurring counter-ligand. In some embodiments, it has at least 60, 70, 80, 90, 95, 99, or 100% sequence identity, or substantial sequence identity, with a naturally occurring counter-ligand for the target ligand.

Target site, as that term is used herein, refers to a site which contains the entity, e.g., epitope, bound by a targeting moiety. In some embodiments, the target site is the site at which immune privilege is established.

Tissue specific targeting moiety, as that term is used herein, refers to a moiety, e.g., an antibody molecule, that as a component of a therapeutic molecule, localizes the therapeutic molecule preferentially to a target tissue, as opposed to other tissue of a subject. As a component of a therapeutic compound, the tissue specific targeting moiety provides site-specific immune privilege for a target tissue, e.g., an organ or tissue undergoing or at risk for autoimmune attack. In some embodiments, a tissue specific targeting moiety binds to a product, e.g., a polypeptide product, which is not present outside the target tissue, or is present at sufficiently low levels that, at therapeutic concentrations of therapeutic molecule, unacceptable levels of immune suppression are absent or substantially absent. In some embodiments, a tissue specific targeting moiety binds to an epitope, which epitope is not present outside, or not substantially present outside, the target site.

In some embodiments, a tissue specific targeting moiety, as a component of a therapeutic compound, preferentially binds to a target tissue or target tissue antigen, e.g., has a binding affinity for the target tissue or antigen that is greater for target antigen or tissue, e.g., at least 2, 4, 5, 10, 50, 100, 500, 1,000, 5,000, or 10,000 fold greater, than its affinity for than for non-target tissue or antigen present outside the target tissue. Affinity of a therapeutic compound of which the tissue specific moiety is a component, can be measured in a cell suspension, e.g., the affinity for suspended cells having the target antigen is compared with its affinity for suspended cells not having the target antigen. In some embodiments, the binding affinity for the target antigen bearing cells is below 10 nM.

In some embodiments, the binding affinity for the target antigen bearing cells is below 100 pM, 50 pM, or 10 pM. In some embodiments, the specificity for a target antigen is sufficient, that when the tissue specific targeting moiety is coupled to an immune-down regulating effector: i) immune attack of the target tissue, e.g., as measured by histological inflammatory response, infiltrating T effector cells, or organ function, in the clinical setting—e.g. creatinine for kidney, is substantially reduced, e.g., as compared to what would be seen in an otherwise similar implant but lacking the tissue specific targeting moiety is coupled to an immune-down regulating effector; and/or ii) immune function in the recipient, outside or away from the target tissue, is substantially maintained.

In some embodiments, one or more of the following is seen: at therapeutic levels of therapeutic compound, peripheral blood lymphocyte counts are not substantially impacted, e.g., the level of T cells is within 25, 50, 75, 85, 90, or 95% of normal, the level of B cells is within 25, 50, 75, 85, 90, or 95% of normal, and/or the level of granulocytes (PMNs) cells is within 25, 50, 75, 85, 90, or 95% of normal, or the level of monocytes is within 25, 50, 75, 85, 90, or 95% of normal 1; at therapeutic levels of therapeutic compound, the ex vivo proliferative function of PBMCs (peripheral blood mononuclear cells) against non-disease relevant antigens is substantially normal or is within 70, 80, or 90% of normal; at therapeutic levels of therapeutic compound, the incidence or risk of risk of opportunistic infections and cancers associated with immunosuppression is not substantially increased over normal; or at therapeutic levels of therapeutic compound, the incidence or risk of risk of opportunistic infections and cancers associated with immunosuppression is substantially less than would be seen with standard of care, or non-targeted, immunosuppression. In some embodiments, the tissue specific targeting moiety comprises an antibody molecule. In some embodiments, the donor specific targeting moiety comprises an antibody molecule, a target specific binding polypeptide, or a target ligand binding molecule. In some embodiments, the tissue specific targeting moiety binds a product, or a site on a product, that is present or expressed exclusively, or substantially exclusively, on target tissue.

ICIM Binding/Modulating Moieties: Effector Binding/Modulating Moieties that Bind Inhibitory Receptors

Methods and compounds described herein provide for a therapeutic compound having an effector binding/modulating moiety comprising an ICIM binding/modulating moiety, that directly binds and activates an inhibitory receptor on the surface of an immune cell, e.g., to reduce or eliminate, or substantially eliminate, the ability of the immune cell to mediate immune attack. Coupling of the ICIM binding/modulating moiety to a targeting entity, promotes site-specific or local down regulation of the immune cell response, e.g., confined substantially to the locations having binding sites for the targeting moiety. Thus, normal systemic immune function is substantially retained. In some embodiments, an ICIM binding/modulating moiety comprises an inhibitory immune checkpoint molecule counter ligand molecule, e.g., a natural ligand, or fragment of a natural ligand (e.g., PD-L1 or HLA-G) of the inhibitory immune checkpoint molecule. In some embodiments, an ICIM binding/modulating moiety comprises a functional antibody molecule, e.g., a functional antibody molecule comprising an scFv binding domain, that engages inhibitory immune checkpoint molecule.

In some embodiments, the ICIM binding/modulating moiety, comprising, e.g., a functional antibody molecule, or inhibitory immune checkpoint molecule ligand molecule, binds the inhibitory receptor but does not prevent binding of a natural ligand of the inhibitory receptor to the inhibitory receptor. In embodiments a format is used wherein a targeting moiety is coupled, e.g., fused, to an ICIM binding/modulating moiety, comprising, e.g., an scFv domain, and configured so that upon binding of an inhibitory receptor while in solution (e.g., in blood or lymph) (and presumably in a monomeric format), the therapeutic molecule: i) fails to agonize, or fails to substantially agonize (e.g., agonizes at less than 30, 20, 15, 10, or 5% of the level seen with a full agonizing molecule) the inhibitory receptor on the immune cell; and/or ii) fails to antagonize, or fails to substantially antagonize (e.g., antagonizes at less than 30, 20, 15, 10, or 5% of the level seen with a full antagonizing molecule) the inhibitory receptor on the immune cell. A candidate molecule can be evaluated for its ability to agonize or not agonize by its ability to either increase or decrease the immune response in an in vitro cell based assay wherein the target is not expressed, e.g., using an MLR-based assay (mixed lymphocyte reaction).

In some embodiments, candidate ICIM binding/modulating moieties can reduce, completely or substantially eliminate systemic immunosuppression and systemic immune activation. In some embodiments, the targeting domain of the therapeutic compound, when bound to target, will serve to cluster or multimerize the therapeutic compound on the surface of the tissue desiring immune protection. In some embodiments, the ICIM binding/modulating moiety, e.g., an ICIM binding/modulating moiety comprising a scFv domain, requires a clustered or multimeric state to be able to deliver an agonistic and immunosuppressive signal, or substantial levels of such signal, to local immune cells. This type of therapeutic can, for example, provide to a local immune suppression whilst leaving the systemic immune system unperturbed or substantially unperturbed. That is, the immune suppression is localized to where the suppression is needed as opposed to being systemic and not localized to a particular area or tissue type.

In some embodiments, upon binding to the target e.g., a target organ, tissue or cell type, the therapeutic compound coats the target, e.g., target organ, tissue or cell type. When circulating lymphocytes attempt to engage and destroy the target, this therapeutic will provide an ‘off’ signal only at, or to a greater extent at, the site of therapeutic compound accumulation.

A candidate therapeutic compound can be evaluated for the ability to bind, e.g., specifically bind, its target, e.g., by ELISA, a cell based assay, or surface plasmon resonance. This property should generally be maximized, as it mediates the site-specificity and local nature of the immune privilege. A candidate therapeutic compound can be evaluated for the ability to down regulate an immune cell when bound to target, e.g., by a cell based activity assay. This property should generally be maximized, as it mediates the site-specificity and local nature of the immune privilege. The level of down regulation effected by a candidate therapeutic compound in monomeric (or non-bound) form can be evaluated, e.g., by a cell based activity assay. This property should generally be minimized, as could mediate systemic down regulation of the immune system. The level of antagonism of a cell surface inhibitory molecule, e.g., an inhibitory immune checkpoint molecule, effected by a candidate therapeutic compound in monomeric (or non-bound) form can be evaluated, e.g., by, e.g., by a cell based activity assay. This property should generally be minimized, as could mediate systemic unwanted activation of the immune system. Generally, the properties should be selected and balanced to produce a sufficiently robust site specific immune privilege without unacceptable levels of non-site specific agonism or antagonism of the inhibitory immune checkpoint molecule.

### The PD-L1/PD-1 Pathway

As provided for herein, in some embodiments, the effector molecule is a PD-1 binding moiety, such as a PD-1 agonist. Programmed cell death protein 1, (often referred to as PD-1) is a cell surface receptor that belongs to the immunoglobulin superfamily. PD-1 is expressed on T cells and other cell types including, but not limited to, B cells, myeloid cells, dendritic cells, monocytes, T regulatory cells, iNK T cells. PD-1 binds two ligands, PD-L1 and PD-L2, and is an inhibitory immune checkpoint molecule. Engagement with a cognate ligand, PD-L1 or PD-L2, in the context of engagement of antigen loaded MCH with the T Cell Receptor on a T cell minimizes or prevents the activation and function of T cells. The inhibitory effect of PD-1 can include both promoting apoptosis (programmed cell death) in antigen specific T-cells in lymph nodes and reducing apoptosis in regulatory T cells (suppressor T cells).

In some embodiments, a therapeutic compound comprises an ICIM binding/modulating moiety which agonizes PD-1 inhibition. An ICIM binding/modulating moiety can include an inhibitory molecule counter ligand molecule, e.g., comprising a fragment of a ligand of PD-1 (e.g., a fragment of PD-L1 or PD-L2) or another moiety, e.g., a functional antibody molecule, comprising, e.g., an scFv domain that binds PD-1.

In some embodiments, a therapeutic compound comprises a targeting moiety that preferentially binds a donor antigen not present in, or present in substantially lower levels in the subject, e.g., a donor antigen from Table 2, and is localized to donor graft tissue in a subject. In some embodiments, it does not bind, or does not substantially bind, other tissues. In some embodiments, a therapeutic compound can include a targeting moiety that is specific for HLA-A2 and specifically binds donor allograft tissue but does not bind, or does not substantially bind, host tissues. In some embodiments, the therapeutic compound comprises an ICIM binding/modulating moiety, e.g., an inhibitory molecule counter ligand molecule, e.g., comprising a fragment of a ligand of PD-1 (e.g., a fragment of PD-L1 or PD-L2) or another moiety, e.g., a functional antibody molecule, comprising, e.g., an scFv domain that binds PD-1, such that the therapeutic compound, e.g., when bound to target, activates PD-1. The therapeutic compound targets an allograft and provides local immune privilege to the allograft.

In some embodiments, a therapeutic compound comprises a targeting moiety that is preferentially binds to an antigen of Table 3, and is localized to the target in a subject, e.g., a subject having an autoimmune disorder, e.g., an autoimmune disorder of Table 3. In some embodiments, it does not bind, or does not substantially bind, other tissues. In some embodiments, the therapeutic compound comprises an ICIM binding/modulating moiety, e.g., an inhibitory molecule counter ligand molecule, e.g., comprising a fragment of a ligand of PD-1 (e.g., a fragment of PD-L1 or PD-L2) or another moiety, e.g., a functional antibody molecule, comprising, e.g., an scFv domain that binds PD-1, such that the therapeutic compound, e.g., when bound to target, activates PD-1. The therapeutic compound targets a tissue subject to autoimmune attack and provides local immune privilege to the tissue.

PD-L1 and PDL2, or polypeptides derived therefrom, can provide candidate ICIM binding moieties. However, in monomer form, e.g., when the therapeutic compound is circulating in blood or lymph, this molecule could have an undesired effect of antagonizing the PD-L1/PD-1 pathway, and may only agonize the PD-1 pathway when clustered or multimerized on the surface of a target, e.g., a target organ. In some embodiments, a therapeutic compound comprises an ICIM binding/modulating moiety comprising a functional antibody molecule, e.g., a scFv domain, that is inert, or substantially inert, to the PD-1 pathway in a soluble form but which agonizes and drives an inhibitory signal when multimerized (by the targeting moiety) on the surface of a tissue.

IIC Binding/Modulating Moieties: Effector Binding/Modulating Moieties that Recruit Immunosuppressive T Cells

In some embodiments, the composition or therapeutic compound that can be used, for example, to treat Type 1 Diabetes (T1D) comprises an effector binding/modulating moiety, e.g., an IIC binding/modulating moiety, that binds, activates, or retains immunosuppressive cells, e.g., immunosuppressive T cells, at the site mediated by the targeting moiety, providing site-specific immune privilege. The IIC binding/modulating moiety, e.g., an IIC binding/modulating moiety comprising an antibody molecule, comprising, e.g., an scFv binding domain, binds immunosuppressive cell types, e.g., Tregs, e.g., Foxp3+CD25+ Tregs. Organ, tissue or specific cell type tolerance is associated with an overwhelming increase of Tregs proximal and infiltrating the target organ; in embodiments, the methods and compounds described herein synthetically re-create and mimic this physiological state. Upon accumulation of Tregs, an immunosuppressive microenvironment is created that serves to protect the organ of interest from the immune system.

IL-2 Mutein Molecules: IL2 Receptor Binders that Activate Tregs

In some embodiments, the effector molecule is an IL-2 mutein molecule. As that term is used herein, refers to an IL2 variant that binds with high affinity to the CD25 (IL-2R alpha chain) and with low affinity to the other IL-2R signaling components CD122 (IL-2R beta) and CD132 (IL-2R gamma). Such an IL-2 mutein molecule preferentially activates Treg cells. In embodiments, either alone, or as a component of a therapeutic compound, an IL-2 mutein activates Tregs at least 2, 5, 10, or 100 fold more than cytotoxic or effector T cells. Exemplary IL-2 mutein molecules are described in WO2010085495, WO2016/164937, US2014/0286898A1, WO2014153111A2, WO2010/085495, cytotoxic WO2016014428A2, WO2016025385A1, and US20060269515. Muteins disclosed in these references that include additional domains, e.g., an Fc domain, or other domain for extension of half-life can be used in the therapeutic compounds and methods described herein without such additional domains. In another embodiment an IIC binding/modulating moiety comprises an IL-2 mutein, or active fragment thereof, coupled, e.g., fused, to another polypeptide, e.g., a polypeptide that extends in vivo half-life, e.g., an immunoglobulin constant region, or a multimer or dimer thereof, e.g., AMG 592. In an embodiment the therapeutic compound comprises the IL-2 portion of AMG 592. In an embodiment the therapeutic compound comprises the IL-2 portion but not the immunoglobulin portion of AMG 592. In some embodiments, the mutein does not comprise a Fc region. For some IL-2 muteins, the muteins are engineered to contain a Fc region because such region has been shown to increase the half-life of the mutein. In some embodiments, the extended half-life is not necessary for the methods described and embodied herein. In some embodiments, the Fc region that is fused with the IL-2 mutein comprises a N297 mutations, such as, but not limited to, N297A. In some embodiments, the Fc region that is fused with the IL-2 mutein does not comprise a N297 mutation, such as, but not limited to, N297A.

Although examples may be provided herein that demonstrate the use of a MAdCAM-PD-1 bispecific to treat Type 1 diabetes, the PD-1 agonist may be replaced with an IL-2 mutein. IL-2 mutein molecules that preferentially expand or stimulate Treg cells (over cytotoxic T cells) can be also used as an IIC binding/modulating moiety.

In some embodiments, IIC binding/modulating moiety comprises an IL-2 mutein molecule. As used herein, the term “IL-2 mutein molecule” or “IL-2 mutein” refers to an IL-2 variant that preferentially activates Treg cells. In some embodiments, either alone, or as a component of a therapeutic compound, an IL-2 mutein molecule activates Tregs at least 2, 5, 10, or 100 fold more than cytotoxic T cells. A suitable assay for evaluating preferential activation of Treg cells can be found in U.S. Pat. No. 9,580,486 at, for example, Examples 2 and 3, or in WO2016014428 at, for example, Examples 3, 4, and 5, each of which is incorporated by reference in its entirety. The sequence of mature IL-2 is

(SEQ ID NO: 6)
APTSSSTKKTQLQLEHLLLDLQMILNGINNYKNPKLTRMLTFKF
YMPKKATELKHLQCLEEELKPLEEVLNLAQSKNFHLRPRDLISN
INVIVLELKGSETTFMCEYADETATIVEFLNRWITFCQSIISTL
T (mature IL-2 sequence).

The immature sequence of IL-2 can be represented by

(SEQ ID NO: 15)
MYRMQLLSCIALSLALVTNSAPTSSSTKKTQLQLEHLLLDLQMI
LNGINNYKNPKLTRMLTFKFYMPKKATELKHLQCLEEELKPLEE
VLNLAQSKNFHLRPRDLISNINVIVLELKGSETTFMCEYADETA
TIVEFLNRWITFCQSIISTLT.

In some embodiments, an IIC binding/modulating moiety comprises an IL-2 mutein, or active fragment thereof, coupled, e.g., fused, to another polypeptide, e.g., a polypeptide that extends in vivo half-life, e.g., an immunoglobulin constant region, or a multimer or dimer thereof.

An IL-2 mutein molecule can be prepared by mutating one or more of the residues of IL-2. Non-limiting examples of IL-2-muteins can be found in WO2016/164937, U.S. Pat. Nos. 9,580,486, 7,105,653, 9,616,105, 9,428,567, US2017/0051029, US2014/0286898A1, WO2014153111A2, WO2010/085495, WO2016014428A2, WO2016025385A1, and US20060269515, each of which are incorporated by reference in its entirety.

In some embodiments, the alanine at position 1 of the sequence above is deleted. In some embodiments, the IL-2 mutein molecule comprises a serine substituted for cysteine at position 125 of the mature IL-2 sequence. Other combinations of mutations and substitutions that are IL-2 mutein molecules are described in US20060269515, which is incorporated by reference in its entirety. In some embodiments, the cysteine at position 125 is also substituted with a valine or alanine. In some embodiments, the IL-2 mutein molecule comprises a V91K substitution. In some embodiments, the IL-2 mutein molecule comprises a N88D substitution. In some embodiments, the IL-2 mutein molecule comprises a N88R substitution. In some embodiments, the IL-2 mutein molecule comprises a substitution of H16E, D84K, V91N, N88D, V91K, or V91R, any combinations thereof. In some embodiments, these IL-2 mutein molecules also comprise a substitution at position 125 as described herein. In some embodiments, the IL-2 mutein molecule comprises one or more substitutions selected from the group consisting of: T3N, T3A, L12G, L12K, L12Q, L12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20H, D20I, D20Y, D20F, D20G, D20T, D20W, M23R, R81A, R81G, R81S, R81T, D84A, D84E, D84G, D84I, D84M, D84Q D84R, D84S, D84T, S87R, N88A, N88D, N88E, N88I, N88F, N88G, N88M, N88R, N88S, N88V, N88W, V91D, V91E, V91G, V91S, I92K, I92R, E95G, and Q126. In some embodiments, the amino acid sequence of the IL-2 mutein molecule differs from the amino acid sequence set forth in mature IL-2 sequence with a C125A or C125S substitution and with one substitution selected from T3N, T3A, L12G, L12K, L12Q L12S, Q13G, E15A, E15G, E15S, H16A, H16D, H16G, H16K, H16M, H16N, H16R, H16S, H16T, H16V, H16Y, L19A, L19D, L19E, L19G, L19N, L19R, L19S, L19T, L19V, D20A, D20E, D20F, D20G, D20T, D20W, M23R, R81A, R81G, R81S, R81T, D84A, D84E, D84G, D84I, D84M, D84Q, D84R, D84S, D84T, S87R, N88A, N88D, N88E, N88F, N88I, N88G, N88M, N88R, N88S, N88V, N88W, V91D, V91E, V91G, V91S, I92K, I92R, E95G, Q126I, Q126L, and Q126F. In some embodiments, the IL-2 mutein molecule differs from the amino acid sequence set forth in mature IL-2 sequence with a C125A or C125S substitution and with one substitution selected from D20H, D20I, D20Y, D20E, D20G, D20W, D84A, D84S, H16D, H16G, H16K, H16R, H16T, H16V, I92K, I92R, L12K, L19D, L19N, L19T, N88D, N88R, N88S, V91D, V91G, V91K, and V91S. In some embodiments, the IL-2 mutein comprises N88R and/or D20H mutations.

In some embodiments, the IL-2 mutein molecule comprises a mutation in the polypeptide sequence at a position selected from the group consisting of amino acid 30, amino acid 31, amino acid 35, amino acid 69, and amino acid 74. In some embodiments, the mutation at position 30 is N30S. In some embodiments, the mutation at position 31 is Y311H. In some embodiments, the mutation at position 35 is K35R. In some embodiments, the mutation at position 69 is V69A. In some embodiments, the mutation at position 74 is Q74P. In some embodiments, the mutein comprises a V69A mutation, a Q74P mutation, a N88D or N88R mutation, and one or more of L53I, L56I, L80I, or L118I mutations. In some embodiments, the mutein comprises a V69A mutation, a Q74P mutation, a N88D or N88R mutation, and a L to I mutation selected from the group consisting of L53I, L56I, L80I, and L118I mutation. In some embodiments, the IL-2 mutein comprises a V69A, a Q74P, a N88D or N88R mutation, and a L53I mutation. In some embodiments, the IL-2 mutein comprises a V69A, a Q74P, a N88D or N88R mutation, and a L56I mutation. In some embodiments, the IL-2 mutein comprises a V69A, a Q74P, a N88D or N88R mutation, and a L80I mutation. In some embodiments, the IL-2 mutein comprises a V69A, a Q74P, a N88D or N88R mutation, and a L1181 mutation. As provided for herein, the muteins can also comprise a C125A or C125S mutation.

In some embodiments, the mutein comprises a T3A mutation. The full length IL-2 muteins provided herein may not be illustrated with a T3A or other mutations provided for herein, but such mutations can be added into the muteins provided herein as is the case for any of the other mutations illustrated herein. Accordingly, In some embodiments, the mutein comprises a T3N mutation. In some embodiments, the mutein comprises a T3A mutation. In some embodiments, the mutein comprises a L12G mutation. In some embodiments, the mutein comprises a L12K mutation. In some embodiments, the mutein comprises a L12Q mutation. In some embodiments, the mutein comprises a L12S mutation. In some embodiments, the mutein comprises a Q13G mutation. In some embodiments, the mutein comprises a E15A mutation. In some embodiments, the mutein comprises a E15G mutation. In some embodiments, the mutein comprises a E15S mutation. In some embodiments, the mutein comprises a H16A mutation. In some embodiments, the mutein comprises a H16D mutation. In some embodiments, the mutein comprises a H16G mutation. In some embodiments, the mutein comprises a H16K mutation. In some embodiments, the mutein comprises a H16M mutation. In some embodiments, the mutein comprises a H16N mutation. In some embodiments, the mutein comprises a H16R mutation. In some embodiments, the mutein comprises a H16S mutation. In some embodiments, the mutein comprises a H16T mutation. In some embodiments, the mutein comprises a H16V mutation. In some embodiments, the mutein comprises a H16Y mutation. In some embodiments, the mutein comprises a L19A mutation. In some embodiments, the mutein comprises a L19D mutation. In some embodiments, the mutein comprises a L19E mutation. In some embodiments, the mutein comprises a L19G mutation. In some embodiments, the mutein comprises a L19N mutation. In some embodiments, the mutein comprises a L19R mutation. In some embodiments, the mutein comprises a L19S mutation. In some embodiments, the mutein comprises a L19T mutation. In some embodiments, the mutein comprises a L19V mutation. In some embodiments, the mutein comprises a D20A mutation. In some embodiments, the mutein comprises a D20E mutation. In some embodiments, the mutein comprises a D20H mutation. In some embodiments, the mutein comprises a D20I mutation. In some embodiments, the mutein comprises a D20Y mutation. In some embodiments, the mutein comprises a D20F mutation. In some embodiments, the mutein comprises a D20G mutation. In some embodiments, the mutein comprises a D20T mutation. In some embodiments, the mutein comprises a D20W mutation. In some embodiments, the mutein comprises a M23R mutation. In some embodiments, the mutein comprises a R81A mutation. In some embodiments, the mutein comprises a R81G mutation. In some embodiments, the mutein comprises a R81S mutation. In some embodiments, the mutein comprises a R81T mutation. In some embodiments, the mutein comprises a D84A mutation. In some embodiments, the mutein comprises a D84E mutation. In some embodiments, the mutein comprises a D84G mutation. In some embodiments, the mutein comprises a D84I mutation. In some embodiments, the mutein comprises a D84M mutation. In some embodiments, the mutein comprises a D84Q mutation. In some embodiments, the mutein comprises a D84R mutation. In some embodiments, the mutein comprises a D84S mutation. In some embodiments, the mutein comprises a D84T mutation. In some embodiments, the mutein comprises a S87R mutation. In some embodiments, the mutein comprises a N88A mutation. In some embodiments, the mutein comprises a N88D mutation. In some embodiments, the mutein comprises a N88E mutation. In some embodiments, the mutein comprises a N88I mutation. In some embodiments, the mutein comprises a N88F mutation. In some embodiments, the mutein comprises a N88G mutation. In some embodiments, the mutein comprises a N88M mutation. In some embodiments, the mutein comprises a N88R mutation. In some embodiments, the mutein comprises a N88S mutation. In some embodiments, the mutein comprises a N88V mutation. In some embodiments, the mutein comprises a N88W mutation. In some embodiments, the mutein comprises a V91D mutation. In some embodiments, the mutein comprises a V91E mutation. In some embodiments, the mutein comprises a V91G mutation. In some embodiments, the mutein comprises a V91S mutation. In some embodiments, the mutein comprises a I92K mutation. In some embodiments, the mutein comprises a I92R mutation. In some embodiments, the mutein comprises a E95G mutation. In some embodiments, the mutein comprises a Q126 mutation.

Although the mutations are illustrated in list form, this is simply for convenience and the muteins may have one or more of the substitutions provided herein.

In some embodiments, the IL-2 mutein molecule comprises a substitution selected from the group consisting of: N88R, N88I, N88G, D20H, D109C, Q126L, Q126F, D84G, or D84I relative to mature human IL-2 sequence provided above. In some embodiments, the IL-2 mutein molecule comprises a substitution of D109C and one or both of a N88R substitution and a C125S substitution. In some embodiments, the cysteine that is in the IL-2 mutein molecule at position 109 is linked to a polyethylene glycol moiety, wherein the polyethylene glycol moiety has a molecular weight of between 5 and 40 kDa.

In some embodiments, any of the substitutions described herein are combined with a substitution at position 125. The substitution can be a C125S, C125A, or C125V substitution.

In addition to the substitutions or mutations described herein, in some embodiments, the IL-2 mutein has a substitution/mutation at one or more of positions 73, 76, 100, or 138 that correspond to SEQ ID NO: 15 or positions at one or more of positions 53, 56, 80, or 118 that correspond to SEQ ID NO: 6. In some embodiments, the IL-2 mutein comprises a mutation at positions 73 and 76; 73 and 100; 73 and 138; 76 and 100; 76 and 138; 100 and 138; 73, 76, and 100; 73, 76, and 138; 73, 100, and 138; 76, 100 and 138; or at each of 73, 76, 100, and 138 that correspond to SEQ ID NO: 15. In some embodiments, the IL-2 mutein comprises a mutation at positions 53 and 56; 53 and 80; 53 and 118; 56 and 80; 56 and 118; 80 and 118; 53, 56, and 80; 53, 56, and 118; 53, 80, and 118; 56, 80 and 118; or at each of 53, 56, 80, and 118 that correspond to SEQ ID NO: 6. As the IL-2 can be fused or tethered to other proteins, as used herein, the term corresponds to as reference to a SEQ ID NOs: 6 or 15 refer to how the sequences would align with default settings for alignment software, such as can be used with the NCBI website. In some embodiments, the mutation is leucine to isoleucine. Thus, the IL-2 mutein can comprise one more isoleucines at positions 73, 76, 100, or 138 that correspond to SEQ ID NO: 15 or positions at one or more of positions 53, 56, 80, or 118 that correspond to SEQ ID NO: 6. In some embodiments, the mutein comprises a mutation at L53 that correspond to SEQ ID NO: 6. In some embodiments, the mutein comprises a mutation at L56 that correspond to SEQ ID NO: 6. In some embodiments, the mutein comprises a mutation at L80 that correspond to SEQ ID NO: 6. In some embodiments, the mutein comprises a mutation at L118 that correspond to SEQ ID NO: 6. In some embodiments, the mutation is leucine to isoleucine. In some embodiments, the mutein also comprises a mutation as position 69, 74, 88, 125, or any combination thereof in these muteins that correspond to SEQ ID NO: 6. In some embodiments, the mutation is a V69A mutation. In some embodiments, the mutation is a Q74P mutation. In some embodiments, the mutation is a N88D or N88R mutation. In some embodiments, the mutation is a C125A or C125S mutation.

In some embodiments, the IL-2 mutein comprises a mutation at one or more of positions 49, 51, 55, 57, 68, 89, 91, 94, 108, and 145 that correspond to SEQ ID NO: 15 or one or more positions 29, 31, 35, 37, 48, 69, 71, 74, 88, and 125 that correspond to SEQ ID NO: 6. The substitutions can be used alone or in combination with one another. In some embodiments, the IL-2 mutein comprises substitutions at 2, 3, 4, 5, 6, 7, 8, 9, or each of positions 49, 51, 55, 57, 68, 89, 91, 94, 108, and 145. Non-limiting examples such combinations include, but are not limited to, a mutation at positions 49, 51, 55, 57, 68, 89, 91, 94, 108, and 145; 49, 51, 55, 57, 68, 89, 91, 94, and 108; 49, 51, 55, 57, 68, 89, 91, and 94; 49, 51, 55, 57, 68, 89, and 91; 49, 51, 55, 57, 68, and 89; 49, 51, 55, 57, and 68; 49, 51, 55, and 57; 49, 51, and 55; 49 and 51; 51, 55, 57, 68, 89, 91, 94, 108, and 145; 51, 55, 57, 68, 89, 91, 94, and 108; 51, 55, 57, 68, 89, 91, and 94; 51, 55, 57, 68, 89, and 91; 51, 55, 57, 68, and 89; 55, 57, and 68; 55 and 57; 55, 57, 68, 89, 91, 94, 108, and 145; 55, 57, 68, 89, 91, 94, and 108; 55, 57, 68, 89, 91, and 94; 55, 57, 68, 89, 91, and 94; 55, 57, 68, 89, and 91; 55, 57, 68, and 89; 55, 57, and 68; 55 and 57; 57, 68, 89, 91, 94, 108, and 145; 57, 68, 89, 91, 94, and 108; 57, 68, 89, 91, and 94; 57, 68, 89, and 91; 57, 68, and 89; 57 and 68; 68, 89, 91, 94, 108, and 145; 68, 89, 91, 94, and 108; 68, 89, 91, and 94; 68, 89, and 91; 68 and 89; 89, 91, 94, 108, and 145; 89, 91, 94, and 108; 89, 91, and 94; 89 and 91; 91, 94, 108, and 145; 91, 94, and 108; 91, and 94; or 94 and 108. Each mutation can be combined with one another. The same substitutions can be made in SEQ ID NO: 6, but the numbering would adjust appropriately as is clear from the present disclosure (20 less than the numbering for SEQ ID NO: 15 corresponds to the positions in SEQ ID NO: 6).

In some embodiments, the IL-2 mutein comprises a mutation at one or more positions of 35, 36, 42, 104, 115, or 146 that correspond to SEQ ID NO: 15 or the equivalent positions at SEQ ID NO: 6 (e.g. positions 15, 16, 22, 84, 95, or 126). These mutations can be combined with the other leucine to isoleucine mutations described herein or the mutation at positions 73, 76, 100, or 138 that correspond to SEQ ID NO: 15 or at one or more of positions 53, 56, 80, or 118 that correspond to SEQ ID NO: 6. In some embodiments, the mutation is a E35Q, H36N, Q42E, D104N, E115Q, or Q146E, or any combination thereof. In some embodiments, one or more of these substitutions is wild type. In some embodiments, the mutein comprises a wild-type residue at one or more of positions 35, 36, 42, 104, 115, or 146 that correspond to SEQ ID NO: 15 or the equivalent positions at SEQ ID NO: 6 (e.g. positions 15, 16, 22, 84, 95, and 126).

The mutations at these positions can be combined with any of the other mutations described herein, including, but not limited to substitutions at positions 73, 76, 100, or 138 that correspond to SEQ ID NO: 15 or positions at one or more of positions 53, 56, 80, or 118 that correspond to SEQ ID NO: 6 described herein and above. In some embodiments, the IL-2 mutein comprises a N49S mutation that corresponds to SEQ ID NO: 15. In some embodiments, the IL-2 mutein comprises a Y51S or a Y51H mutation that corresponds to SEQ ID NO: 15. In some embodiments, the IL-2 mutein comprises a K55R mutation that corresponds to SEQ ID NO: 15. In some embodiments, the IL-2 mutein comprises a T57A mutation that corresponds to SEQ ID NO: 15. In some embodiments, the IL-2 mutein comprises a K68E mutation that corresponds to SEQ ID NO: 15. In some embodiments, the IL-2 mutein comprises a V89A mutation that corresponds to SEQ ID NO: 15. In some embodiments, the IL-2 mutein comprises a N91R mutation that corresponds to SEQ ID NO: 15. In some embodiments, the IL-2 mutein comprises a Q94P mutation that corresponds to SEQ ID NO: 15. In some embodiments, the IL-2 mutein comprises a N108D or a N108R mutation that corresponds to SEQ ID NO: 15. In some embodiments, the IL-2 mutein comprises a C145A or C145S mutation that corresponds to SEQ ID NO: 15. These substitutions can be used alone or in combination with one another. In some embodiments, the mutein comprises each of these substitutions. In some embodiments, the mutein comprises 1, 2, 3, 4, 5, 6, 7, or 8 of these mutations. In some embodiments, the mutein comprises a wild-type residue at one or more of positions 35, 36, 42, 104, 115, or 146 that correspond to SEQ ID NO: 15 or the equivalent positions at SEQ ID NO: 6 (e.g. positions 15, 16, 22, 84, 95, and 126).

In some embodiments, the IL-2 mutein comprises a N29S mutation that corresponds to SEQ ID NO: 6. In some embodiments, the IL-2 mutein comprises a Y31S or a Y31H mutation that corresponds to SEQ ID NO: 6. In some embodiments, the IL-2 mutein comprises a K35R mutation that corresponds to SEQ ID NO: 6. In some embodiments, the IL-2 mutein comprises a T37A mutation that corresponds to SEQ ID NO: 6. In some embodiments, the IL-2 mutein comprises a K48E mutation that corresponds to SEQ ID NO: 6. In some embodiments, the IL-2 mutein comprises a V69A mutation that corresponds to SEQ ID NO: 6. In some embodiments, the IL-2 mutein comprises a N71R mutation that corresponds to SEQ ID NO: 6. In some embodiments, the IL-2 mutein comprises a Q74P mutation that corresponds to SEQ ID NO: 6. In some embodiments, the IL-2 mutein comprises a N88D or a N88R mutation that corresponds to SEQ ID NO: 6. In some embodiments, the IL-2 mutein comprises a C125A or C125S mutation that corresponds to SEQ ID NO: 6. These substitutions can be used alone or in combination with one another. In some embodiments, the mutein comprises 1, 2, 3, 4, 5, 6, 7, or 8 of these mutations. In some embodiments, the mutein comprises each of these substitutions. In some embodiments, the mutein comprises a wild-type residue at one or more of positions 35, 36, 42, 104, 115, or 146 that correspond to SEQ ID NO: 15 or the equivalent positions at SEQ ID NO: 6 (e.g., positions 15, 16, 22, 84, 95, and 126).

For any of the IL-2 muteins described herein, in some embodiments, one or more of positions 35, 36, 42, 104, 115, or 146 that correspond to SEQ ID NO: 15 or the equivalent positions at SEQ ID NO: 6 (e.g. positions 15, 16, 22, 84, 95, or 126) are wild-type (e.g., are as shown in SEQ ID NOs: 6 or 15). In some embodiments, 2, 3, 4, 5, 6, or each of positions 35, 36, 42, 104, 115, or 146 that correspond to SEQ ID NO: 15 or the equivalent positions at SEQ ID NO: 6 (e.g. positions 15, 16, 22, 84, 95, and 126) are wild-type.

In some embodiments, the IL-2 mutein comprises a sequence of:

(SEQ ID NO: 16)
MYRMQLLSCIALSLALVTNSAPTSSSTKKTQLQLEHLLLDLQMI
LNGISNHKNPRLARMLTFKFYMPEKATEIKHLQCLEEELKPLEE
ALRLAPSKNFHLRPRDLISDINVIVLELKGSETTFMCEYADETA
TIVEFLNRWITFSQSIISTLT.

In some embodiments, the IL-2 mutein comprises a sequence of:

(SEQ ID NO: 17)
MYRMQLLSCIALSLALVTNSAPTSSSTKKTQLQLEHLLLDLQMI
LNGISNHKNPRLARMLTFKFYMPEKATELKHIQCLEEELKPLEE
ALRLAPSKNFHLRPRDLISDINVIVLELKGSETTFMCEYADETA
TIVEFLNRWITFSQSIISTLT.

In some embodiments, the IL-2 mutein comprises a sequence of:

(SEQ ID NO: 18)
MYRMQLLSCIALSLALVTNSAPTSSSTKKTQLQLEHLLLDLQMI
LNGISNHKNPRLARMLTFKFYMPEKATELKHLQCLEEELKPLEE
ALRLAPSKNFHIRPRDLISDINVIVLELKGSETTFMCEYADETA
TIVEFLNRWITFSQSIISTLT.

In some embodiments, the IL-2 mutein comprises a sequence of:

(SEQ ID NO: 19)
MYRMQLLSCIALSLALVTNSAPTSSSTKKTQLQLEHLLLDLQMI
LNGISNHKNPRLARMLTFKFYMPEKATELKHLQCLEEELKPLEE
ALRLAPSKNFHLRPRDLISDINVIVLELKGSETTFMCEYADETA
TIVEFINRWITFSQSIISTLT.

In some embodiments, the IL-2 mutein sequences described herein do not comprise the IL-2 leader sequence. The IL-2 leader sequence can be represented by the sequence of MYRMQLLSCIALSLALVTNS (SEQ ID NO: 20). Therefore, in some embodiments, the sequences illustrated above can also encompass peptides without the leader sequence. Although SEQ ID NOs: 16-20 are illustrated with only mutation at one of positions 73, 76, 100, or 138 that correspond to SEQ ID NO: 15 or positions at one or more of positions 53, 56, 80, or 118 that correspond to SEQ ID NO: 6, the peptides can comprise one, two, three or 4 of the mutations at these positions. In some embodiments, the substitution at each position is isoleucine or other type of conservative amino acid substitution. In some embodiments, the leucine at the recited positions are substituted with, independently, isoleucine, valine, methionine, or phenylalanine.

In some embodiments, the IL-2 mutein molecule is fused to a Fc Region or other linker region as described herein. Examples of such fusion proteins can be found in U.S. Pat. Nos. 9,580,486, 7,105,653, 9,616,105, 9,428,567, US2017/0051029, WO2016/164937, US2014/0286898A1, WO2014153111A2, WO2010/085495, WO2016014428A2, WO2016025385A1, US2017/0037102, and US2006/0269515, each of which are incorporated by reference in its entirety.

In some embodiments, the Fc Region comprises what is known as the LALA mutation. Using the Kabat numbering of the Fc region, this would correspond to L247A, L248A, and G250A. In some embodiments, using the EU numbering of the Fc region, the Fc region comprises a L234A mutation, a L235A mutation, and/or a G237A mutation. Regardless of the numbering system used, in some embodiments, the Fc portion can comprise mutations that correspond to these residues. In some embodiments, the Fe Region comprises N297G or N297A (kabat numbering) mutations. The Kabat numbering is based upon a full-length sequence, but would be used in a fragment based upon a traditional alignment used by one of skill in the art for the Fc region.

In some embodiments, the Fc Region comprises a sequence of:

(SEQ ID NO: 21)
DKTHTCPPCPAPEAAGAPSVFLFPPKPKDTLMISRTPEVTCVVV
DVSHEDPEVKFNWYVDGVEVHNAKTKPREEQYNSTYRVVSVLTV
LHQDWLNGKEYKCKVSNKALPAPIEKTISKAKGQPREPQVYTLP
PSREEMTKNQVSLTCLVKGFYPSDIAVEWESNGQPENNYKTTPP
VLDSDGSFFLYSKLTVDKSRWQQGNVFSCSVMHEALHNHYTQKS
LSLSPG;
or
(SEQ ID NO: 28)
DKTHTCPPCPAPELLGGPSVFLFPPKPKDTLMISRTPEVTCVVV
DVSHEDPEVKFNWYVDGVEVHNAKTKPREEQYNSTYRVVSVLTV
LHQDWLNGKEYKCKVSNKALPAPIEKTISKAKGQPREPQVYTLP
PSREEMTKNQVSLTCLVKGFYPSDIAVEWESNGQPENNYKTTPP
VLDSDGSFFLYSKLTVDKSRWQQGNVFSCSVMHEALHNHYTQKS
LSLSPG.

In some embodiments, the IL-2 mutein is linked to the Fc Region. Non-limiting examples of linkers are glycine/serine linkers. For example, a glycine/serine linkers can be a sequence of GGGGSGGGGSGGGGSGGGGS (SEQ ID NO: 22) or GGGGSGGGGSGGGGS (SEQ ID NO: 30). This is simply a non-limiting example and the linker can have varying number of GGGGS (SEQ ID NO: 23) or GGGGA repeats (SEQ ID NO: 29). In some embodiments, the linker comprises 1, 2, 3, 4, 5, 6, 7, 8, 9, or 10 of the GGGGS (SEQ ID NO: 23) or GGGGA repeats (SEQ ID NO: 29) repeats (repeats disclosed as SEQ ID NOS 1550-1551, respectively). In some embodiments, the linker is 10 amino acids in length. In some embodiments, the linker is 5 amino acids in length. In some embodiments, the linker is 15 amino acids in length. In some embodiments, the linker is 20 amino acids in length. In some embodiments, the linker is 25 amino acids in length. In some embodiments, the linker is 30 amino acids in length. In some embodiments, the linker is 35 amino acids in length. In some embodiments, the linker is from 5-50 amino acids in length.

Thus, the IL-2/Fc Fusion can be represented by the formula of Z_{IL-2M}-L_{gs}-Z_{Fc}, wherein Z_{IL-2M} is an IL-2 mutein as described herein, L_{gs} is a linker sequence as described herein (e.g. glycine/serine linker) and Z_{Fc} is a Fc region described herein or known to one of skill in the art. In some embodiments, the formula can be in the reverse orientation Z_{Fc}-L_{gs}-Z_{IL-2M}.

In some embodiments, the IL-2/Fc fusion comprises a sequence of

(SEQ ID NO: 24)
MYRMQLLSCIALSLALVTNSAPTSSSTKKTQLQLEHLLLDLQMI
LNGISNHKNPRLARMLTFKFYMPEKATEIKHLQCLEEELKPLEE
ALRLAPSKNFHLRPRDLISDINVIVLELKGSETTFMCEYADETA
TIVEFLNRWITFSQSIISTLTGGGGSGGGGSGGGGSGGGGSDKT
HTCPPCPAPEAAGAPSVFLFPPKPKDTLMISRTPEVTCVVVDVS
HEDPEVKFNWYVDGVEVHNAKTKPREEQYNSTYRVVSVLTVLHQ
DWLNGKEYKCKVSNKALPAPIEKTISKAKGQPREPQVYTLPPSR
EEMTKNQVSLTCLVKGFYPSDIAVEWESNGQPENNYKTTPPVLD
SDGSFFLYSKLTVDKSRWQQGNVFSCSVMHEALHNHYTQKSLSL
SPG;
(SEQ ID NO: 25)
MYRMQLLSCIALSLALVTNSAPTSSSTKKTQLQLEHLLLDLQMI
LNGISNHKNPRLARMLTFKFYMPEKATELKHIQCLEEELKPLEE
ALRLAPSKNFHLRPRDLISDINVIVLELKGSETTFMCEYADETA
TIVEFLNRWITFSQSIISTLTGGGGSGGGGSGGGGSGGGGSDKT
HTCPPCPAPEAAGAPSVFLFPPKPKDTLMISRTPEVTCVVVDVS
HEDPEVKFNWYVDGVEVHNAKTKPREEQYNSTYRVVSVLTVLHQ
DWLNGKEYKCKVSNKALPAPIEKTISKAKGQPREPQVYTLPPSR
EEMTKNQVSLTCLVKGFYPSDIAVEWESNGQPENNYKTTPPVLD
SDGSFFLYSKLTVDKSRWQQGNVFSCSVMHEALHNHYTQKSLSL
SPG;
(SEQ ID NO: 26)
MYRMQLLSCIALSLALVTNSAPTSSSTKKTQLQLEHLLLDLQMI
LNGISNHKNPRLARMLTFKFYMPEKATELKHLQCLEEELKPLEE
ALRLAPSKNFHIRPRDLISDINVIVLELKGSETTFMCEYADETA
TIVEFLNRWITFSQSIISTLTGGGGSGGGGSGGGGSGGGGSDKT
HTCPPCPAPEAAGAPSVFLFPPKPKDTLMISRTPEVTCVVVDVS
HEDPEVKFNWYVDGVEVHNAKTKPREEQYNSTYRVVSVLTVLHQ
DWLNGKEYKCKVSNKALPAPIEKTISKAKGQPREPQVYTLPPSR
EEMTKNQVSLTCLVKGFYPSDIAVEWESNGQPENNYKTTPPVLD
SDGSFFLYSKLTVDKSRWQQGNVFSCSVMHEALHNHYTQKSLSL
SPG;
or
(SEQ ID NO: 27)
MYRMQLLSCIALSLALVTNSAPTSSSTKKTQLQLEHLLLDLQMI
LNGISNHKNPRLARMLTFKFYMPEKATELKHLQCLEEELKPLEE
ALRLAPSKNFHLRPRDLISDINVIVLELKGSETTFMCEYADETA
TIVEFINRWITFSQSIISTLTGGGGSGGGGSGGGGSGGGGSDKT
HTCPPCPAPEAAGAPSVELFPPKPKDTLMISRTPEVTCVVVDVS
HEDPEVKFNWYVDGVEVHNAKTKPREEQYNSTYRVVSVLTVLHQ
DWLNGKEYKCKVSNKALPAPIEKTISKAKGQPREPQVYTLPPSR
EEMTKNQVSLTCLVKGFYPSDIAVEWESNGQPENNYKTTPPVLD
SDGSFFLYSKLTVDKSRWQQGNVFSCSVMHEALHNHYTQKSLSL
SPG.

In some embodiments, the IL-2/Fc Fusion comprises a sequence selected from the following table, Table 3.

TABLE 3
IL-2/Fc Fusion Protein Amino Acid Sequences

| Sequence | |
|---|---|
| Identification | Sequence |
| SEQ ID NO: 7 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGAGGGGDKTHTCPPCPAPELLGGPSVFLFPPKPKDTLMISRTPEVTC |
| | VVVDVSHEDPEVKFNWYVDGVEVHNAKTKPREEQYASTYRVVSVLTVLHQDWLNGKEYKCK |
| | VSNKALPAPIEKTISKAKGQPREPQVYTLPPSRDELTKNQVSLTCLVKGFYPSDIAVEWES |
| | NGQPENNYKTTPPVLDSDGSFFLYSKLTVDKSRWQQGNVFSCSVMHEALHNHYTQKSLSLS |
| | PGK |
| SEQ ID NO: 8 | APTSSSTKKTQLQLEHLLLHLQMILNGINNYKNPKLTRMLTFKFYMPKKATELKHLQCLEE |
| | ELKPLEEVLNLAQSKNFHLRPRDLISNINVIVLELKGSETTFMCEYADETATIVEFLNRWI |
| | TFSQSIISTLTVECPPCPAPPVAGPSVFLFPPKPKDTLMISRTPEVTCVVVDVSHEDPEVQ |
| | FNWYVDGVEVHNAKTKPREEQFNSTFRVVSVLTVVHQDWLNGKEYKCKVSNKGLPAPIEKT |
| | ISKTKGQPREPQVYTLPPSREEMTKNQVSLTCLVKGFYPSDIAVEWESNGQPENNYKTTPP |
| | MLDSDGSFFLYSKLTVDKSRWQQGNVFSCSVMHEALHNHYTQKSLSLSPGK |
| SEQ ID NO: 9 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTDKTHTCPPCPAPELLGGPSVFLFPPKPKDTLMISRTPEVTCVVVDVSHED |
| | PEVKFNWYVDGVEVHNAKTKPREEQYASTYRVVSVLTVLHQDWLNGKEYKCKVSNKALPAP |
| | IEKTISKAKGQPREPQVYTLPPSRDELTKNQVSLTCLVKGFYPSDIAVEWESNGQPENNYK |
| | TTPPVLDSDGSFFLYSKLTVDKSRWQQGNVFSCSVMHEALHNHYTQKSLSLSPG |
| SEQ ID NO: 10 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGSDKTHTCPPCPAPELLGGPSVFLFPPKPKDTLMISRTPEVTCVVVD |
| | VSHEDPEVKENWYVDGVEVHNAKTKPREEQYASTYRVVSVLTVLHQDWLNGKEYKCKVSNK |
| | ALPAPIEKTISKAKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHYTQKSLSLSPG |
| SEQ ID NO: 11 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGSGGGGSDKTHTCPPCPAPELLGGPSVFLFPPKPKDTLMISRTPEVT |
| | CVVVDVSHEDPEVKFNWYVDGVEVHNAKTKPREEQYASTYRVVSVLTVLHQDWLNGKEYKC |
| | KVSNKALPAPIEKTISKAKGQPREPQVYTLPPSRDELTKNQVSLTCLVKGFYPSDIAVEWE |
| | SNGQPENNYKTTPPVLDSDGSFFLYSKLTVDKSRWQQGNVFSCSVMHEALHNHYTQKSLSL |
| | SPG |
| SEQ ID NO: 12 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGSGGGGSGGGGSDKTHTCPPCPAPELLGGPSVFLFPPKPKDTLMISR |
| | TPEVTCVVVDVSHEDPEVKFNWYVDGVEVHNAKTKPREEQYASTYRVVSVLTVLHQDWLNG |
| | KEYKCKVSNKALPAPIEKTISKAKGQPREPQVYTLPPSRDELTKNQVSLTCLVKGFYPSDI |
| | AVEWESNGQPENNYKTTPPVLDSDGSFFLYSKLTVDKSRWQQGNVFSCSVMHEALHNYHTQ |
| | KSLSLSPG |
| SEQ ID NO: 13 | APTSSSTKKTQLQLEHLLLDLQMILNGINNYKNPKLTRMLTFKFYMPKKATELKHLQCLEE |
| | ELKPLEEVLNLAQSKNFHLRPRDLISRINVIVLELKGSETTFMCEYADETATIVEFLNRWI |
| | TFSQSIISTLTGGGGSGGGGSGGGGSGGGGSDKTHTCPPCPAPELLGGPSVFLFPPKPKDT |
| | LMISRTPEVTCVVVDVSHEDPEVKFNWYVDGVEVHNAKTKPREEQYASTYRVVSVLTVLHQ |
| | DWLNGKEYKCKVSNKALPAPIEKTISKAKGQPREPQVYTLPPSRDELTKNQVSLTCLVKGF |
| | YPSDIAVEWESNGQPENNYKTTPPVLDSDGSFFLYSKLTVDKSRWQQGNVFSCSVMHEALH |
| | NHYTQKSLSLSPG |
| SEQ ID NO: 14 | APTSSSTKKTQLQLEHLLLHLQMILNGINNYKNPKLTRMLTFKFYMPKKATELKHLQCLEE |
| | ELKPLEEVLNLAQSKNFHLRPRDLISNINVIVLELKGSETTFMCEYADETATIVEFLNRWI |
| | TFSQSIISTLTGGGGSGGGGSGGGGSDKTHTCPPCPAPELLGGPSVFLFPPKPKDTLMISR |
| | TPEVTCVVVDVSHEDPEVKFNWYVDGVEVHNAKTKPREEQYASTYPVVSVLTVLHQDWLNG |
| | KEYKCKVSNKALPAPIEKTISKAKGQPREPQVYTLPPSRDELTKNQVSLTCLVKGFYPSDI |
| | AVEWESNGQPENNYKTTPPVLDSDGSFFLYSKLTVDKSRWQQGNVFSCSVMHEALHNHYTQ |
| | KSLSLSPG |

In some embodiments, the IL-2 muteins comprises one or more of the sequences provided in the following table, which, in some embodiments, shows the IL-2 mutein fused with other proteins or linkers. The table also provides sequences for a variety of Fc domains or variants that the IL-2 can be fused with:

| SEQ ID | Brief | |
|---|---|---|
| NO: | Description | Amino Acid Sequence |
| 31 | Human IL-2 | APTSSSTKKTQLQLEHLLLDLQMILNGINNYKNPKLTRMLTFKFYMPKKA |
| | with C125S | TELKHLQCLEEELKPLEEVLNLAQSKNFHLRPRDLISNINVIVLELKGSE |
| | mutation | TTFMCEYADETATIVEFLNRWITFSQSIISTLT |
| 32 | Human IL-2 | APASSSTKKTQLQLEHLLLDLQMILNGINNYKNPKLTRMLTFKFYMPKKA |
| | with C125S | TELKHLQCLEEELKPLEEVLNLAQSKNFHLRPRDLISNINVIVLELKGSE |
| | and T3A | TTFMCEYADETATIVEFLNRWITFSQSIISTLT |
| | mutations | |
| 33 | Human IL-2 | APTSSSTKKTQLQLEHLLLDLQMILNGINNYKNPKLTRMLTFKFYMPKKA |
| | with N88R and | TELKHLQCLEEELKPLEEVLNLAQSKNFHLRPRDLISRINVIVLELKGSE |
| | C125S | TTFMCEYADETATIVEFLNRWITFSQSIISTLT |
| 34 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISNINVIVLELKGSE |
| | Q74P and | TTFMCEYADETATIVEFLNRWITFSQSIISTLT |
| | C125S | |
| | mutations | |
| 35 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISDINVIVLELKGSE |
| | Q74P, N88D | TTFMCEYADETATIVEFLNRWITFSQSIISTLT |
| | and C125S | |
| | mutations | |
| 36 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISRINVIVLELKGSE |
| | Q74P, N88R | TTFMCEYADETATIVEFLNRWITFSQSIISTLT |
| | and C125S | |
| | mutations | |
| 37 | Human IL-2 | APTSSSTKKTQLQLEHLLLDLQMILNGINNYKNPKLTRMLTFKFYMPKKA |
| | with N88D and | TELKHLQCLEEELKPLEEVLNLAQSKNFHLRPRDLISDINVIVLELKGSE |
| | C125S | TTFMCEYADETATIVEFLNRWITFSQSIISTLT |
| 38 | Human IL-2 | APTSSSTKKTQLQLEHLLLDLQMILNGINNYKNPKLTRMLTFKFYMPKKA |
| | with L53I, | TEIKHLQCLEEELKPLEEALNLAPSKNFHLRPRDLISDINVIVLELKGSE |
| | V69A, Q74P, | TTFMCEYADETATIVEFLNRWITFSQSIISTLT |
| | N88D and | |
| | C125S | |
| | mutations | |
| 39 | Human IL-2 | APTSSSTKKTQLQLEHLLLDLQMILNGINNYKNPKLTRMLTFKFYMPKKA |
| | with L56I, | TELKHIQCLEEELKPLEEALNLAPSKNFHLRPRDLISDINVIVLELKGSE |
| | V69A, Q74P, | TTFMCEYADETATIVEFLNRWITFSQSIISTLT |
| | N88D and | |
| | C125S | |
| | mutations | |
| 40 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHIRPRDLISDINVIVLELKGSE |
| | Q74P, L80I, | TTFMCEYADETATIVEFLNRWITFSQSIISTLT |
| | N88D and | |
| | C125S | |
| | mutations | |
| 41 | Human IL-2 | APTSSSTKKTQLQLEHLLLDLQMILNGINNYKNPKLTRMLTFKFYMPKKA |
| | with V69A, | TELKHLQCLEEELKPLEEALNLAPSKNFHLRPRDLISDINVIVLELKGSE |
| | Q74P, N88D, | TTFMCEYADETATIVEFINRWITFSQSIISTLT |
| | L118I, and | |
| | C125S | |
| | mutations | |
| 21 | Human IgG1 Fc | DKTHTCPPCPAPEAAGAPSVFLFPPKPKDTLMISRTPEVTCVVVDVSHED |
| | (N-terminal | PEVKFNWYVDGVEVHNAKTKPREEQYNSTYRVVSVLTVLHQDWLNGKEYK |
| | fusions) with | CKVSNKALPAPIEKTISKAKGQPREPQVYTLPPSREEMTKNQVSLTCLVK |
| | L234A, L235A, | GFYPSDIAVEWESNGQPENNYKTTPPVLDSDGSFFLYSKLTVDKSRWQQG |
| | and G237A | NVFSCSVMHEALHNHYTQKSLSLSPG |
| | mutations | |
| 30 | GGGGSGGGGSGGG | GGGGSGGGGSGGGGS |
| | GS linker (15 | |
| | amino acids) | |
| 22 | GGGGSGGGGSGGG | GGGGSGGGGSGGGGSGGGGS |
| | GSGGGGS | |
| | linker (20 | |
| | amino acids) | |
| 23 | GGGGS linker | GGGGS |
| | (5 amino | |
| | acids) | |
| 43 | Human IgG1 Fc | DKTHTCPPCPAPELLGGPSVFLFPPKPKDTLMISRTPEVTCVVVDVSHED |
| | (truncated) | PEVKFNWYVDGVEVHNAKTKPREEQYGSTYRVVSVLTVLHQDWLNGKEYK |
| | with N297G | CKVSNKALPAPIEKTISKAKGQPREPQVYTLPPSREEMTKNQVSLTCLVK |
| | mutation | GFYPSDIAVEWESNGQPENNYKTTPPVLDSDGSFFLYSKLTVDKSRWQQG |
| | | NVFSCSVMHEALHNHYTQKSLSLSPG |
| 44 | Antibody | ASTKGPSVFPLAPSSKSTSGGTAALGCLVKDYFPEPVTVSWNSGALTSGV |
| | Heavy Chain | HTFPAVLQSSGLYSLSSVVTVPSSSLGTQTYICNVNHKPSNTKVDKKVEP |
| | CH1-CH2-CH3 | KSCDKTHTCPPCPAPEAAGAPSVFLFPPKPKDTLMISRTPEVTCVVVDVS |
| | domains | HEDPEVKFNWYVDGVEVHNAKTKPREEQYNSTYRVVSVLTVLHQDWLNGK |
| | (human IgG1 | EYKCKVSNKALPAPIEKTISKAKGQPREPQVYTLPPSREEMTKNQVSLTC |
| | with L234A, | LVKGFYPSDIAVEWESNGQPENNYKTTPPVLDSDGSFFLYSKLTVDKSRW |
| | L235A, and | QQGNVFSCSVMHEALHNHYTQKSLSLSPG |
| | G237A) | |
| 45 | Antibody | RTVAAPSVFIFPPSDEQLKSGTASVVCLLNNFYPREAKVQWKVDNALQSG |
| | Kappa | NSQESVTEQDSKDSTYSLSSTLTLSKADYEKHKVYACEVTHQGLSSPVTK |
| | Constant | SFNRGEC |
| | Domain | |
| | (human) | |
| 46 | IL-2-G4Sx3-Fc | APTSSSTKKTQLQLEHLLLDLQMILNGINNYKNPKLTRMLTFKFYMPKKA |
| | | TELKHLQCLEEELKPLEEVLNLAQSKNFHLRPRDLISNINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 47 | IL-2 T3A- | APASSSTKKTQLQLEHLLLDLQMILNGINNYKNPKLTRMLTFKFYMPKKA |
| | G4Sx3-Fc | TELKHLQCLEEELKPLEEVLNLAQSKNFHLRPRDLISNINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 48 | IL-2 N88R- | APTSSSTKKTQLQLEHLLLDLQMILNGINNYKNPKLTRMLTFKFYMPKKA |
| | G4Sx3-Fc | TELKHLQCLEEELKPLEEVLNLAQSKNFHLRPRDLISRINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 49 | IL-2 V69A, | APTSSSTKKTQLQLEHLLLDLQMILNGINNYKNPKLTRMLTFKFYMPKKA |
| | Q74P,-G4Sx3- | TELKHLQCLEEELKPLEEALNLAPSKNFHLRPRDLISNINVIVLELKGSE |
| | Fc | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 50 | IL-2 N88D | APTSSSTKKTQLQLEHLLLDLQMILNGINNYKNPKLTRMLTFKFYMPKKA |
| | V69A, Q74P- | TELKHLQCLEEELKPLEEALNLAPSKNFHLRPRDLISDINVIVLELKGSE |
| | G4Sx3-Fc | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 51 | IL-2 N88R | APTSSSTKKTQLQLEHLLLDLQMILNGINNYKNPKLTRMLTFKFYMPKKA |
| | V69A, Q74P- | TELKHLQCLEEELKPLEEALNLAPSKNFHLRPRDLISRINVIVLELKGSE |
| | G4Sx3-Fc | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 52 | IL-2 N88D- | APTSSSTKKTQLQLEHLLLDLQMILNGINNYKNPKLTRMLTFKFYMPKKA |
| | G4Sx3-Fc | TELKHLQCLEEELKPLEEVLNLAQSKNFHLRPRDLISDINVIVLELKGSE |
| | | TTFMCEYADETATIVEFLNRWITFSQSIISTLTGGGGSGGGGSGGGGSDK |
| | | THTCPPCPAPEAAGAPSVFLFPPKPKDTLMISRTPEVTCVVVDVSHEDPE |
| | | VKFNWYVDGVEVHNAKTKPREEQYNSTYRVVSVLTVLHQDWLNGKEYKCK |
| | | VSNKALPAPIEKTISKAKGQPREPQVYTLPPSREEMTKNQVSLTCLVKGF |
| | | YPSDIAVEWESNGQPENNYKTTPPVLDSDGSFFLYSKLTVDKSRWQQGNV |
| | | FSCSVMHEALHNHYTQKSLSLSPG |
| 53 | IL-2 L53I | APTSSSTKKTQLQLEHLLLDLQMILNGINNYKNPKLTRMLTFKFYMPKKA |
| | N88D V69A, | TEIKHLQCLEEELKPLEEALNLAPSKNFHLRPRDLISDINVIVLELKGSE |
| | Q74P,C125S- | TTFMCEYADETATIVEFLNRWITFSQSIISTLTGGGGSGGGGSGGGGSGG |
| | G4Sx4-Fc | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 54 | IL-2 L56I | APTSSSTKKTQLQLEHLLLDLQMILNGINNYKNPKLTRMLTFKFYMPKKA |
| | N88D V69A, | TELKHIQCLEEELKPLEEALNLAPSKNFHLRPRDLISDINVIVLELKGSE |
| | Q74P, C125S- | TTFMCEYADETATIVEFLNRWITFSQSIISTLTGGGGSGGGGSGGGGSGG |
| | G4Sx4-Fc | GGSDKTHTCPPCPAPEAAGAPSVELFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 55 | IL-2 L80I | APTSSSTKKTQLQLEHLLLDLQMILNGINNYKNPKLTRMLTFKFYMPKKA |
| | N88D V69A, | TELKHLQCLEEELKPLEEALNLAPSKNFHIRPRDLISDINVIVLELKGSE |
| | C125S Q74P- | TTFMCEYADETATIVEFLNRWITFSQSIISTLTGGGGSGGGGSGGGGSGG |
| | G4Sx4-Fc | GGSDKTHTCPPCPAPEAAGAPSVELFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 56 | IL-2 L118I | APTSSSTKKTQLQLEHLLLDLQMILNGINNYKNPKLTRMLTFKFYMPKKA |
| | N88D V69A, | TELKHLQCLEEELKPLEEALNLAPSKNFHLRPRDLISDINVIVLELKGSE |
| | Q74P, C125S- | TTFMCEYADETATIVEFINRWITFSQSIISTLTGGGGSGGGGSGGGGSGG |
| | G4Sx4-Fc | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 57 | IL-2 N88D | APTSSSTKKTQLQLEHLLLDLQMILNGINNYKNPKLTRMLTFKFYMPKKA |
| | V69A, Q74P- | TELKHLQCLEEELKPLEEALNLAPSKNFHLRPRDLISDINVIVLELKGSE |
| | G4Sx4-Fc | TTFMCEYADETATIVEFLNRWITFSQSIISTLTGGGGSGGGGSGGGGSGG |
| | | GGSDKTHTCPPCPAPEAAGAPSVFLFPPKPKDTLMISRTPEVTCVVVDVS |
| | | HEDPEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRW |
| | | QQGNVFSCSVMHEALHNHYTQKSLSLSPG |
| 58 | Fc-G4S-IL-2 | DKTHTCPPCPAPELLGGPSVFLFPPKPKDTLMISRTPEVTCVVVDVSHED |
| | N88D V69A, | PEVKFNWYVDGVEVHNAKTKPREEQYGSTYRVVSVLTVLHQDWLNGKEYK |
| | Q74P | CKVSNKALPAPIEKTISKAKGQPREPQVYTLPPSREEMTKNQVSLTCLVK |
| | | GFYPSDIAVEWESNGQPENNYKTTPPVLDSDGSFFLYSKLTVDKSRWQQG |
| | | NVFSCSVMHEALHNHYTQKSLSLSPGGGGGSAPTSSSTKKTQLQLEHLLL |
| | | DLQMILNGINNYKNPKLTRMLTFKFYMPKKATELKHLQCLEEELKPLEEA |
| | | LNLAPSKNFHLRPRDLISDINVIVLELKGSETTFMCEYADETATIVEFLN |
| | | RWITFAQSIISTLT |
| 59 | IL-2 N88D | APTSSSTKKTQLQLEHLLLDLQMILNGINNYKNPKLTRMLTFKFYMPKKA |
| | V69A, Q74P, | TEX_{1K}HX₂QCLEEELKPLEEALNLAPSKNFHX₃RPRDLISDINVIVLELKG |
| | C125S-G4Sx4- | SETTFMCEYADETATIVEFX₄NRWITFSQSIISTLTGGGGSGGGGSGGGGS |
| | Fc, wherein | GGGGSDKTHTCPPCPAPEAAGAPSVFLFPPKPKDTLMISRTPEVTCVVVD |
| | at least one | VSHEDPEVKFNWYVDGVEVHNAKTKPREEQYNSTYRVVSVLTVLHQDWLN |
| | of X₁, X₂, X₃, | GKEYKCKVSNKALPAPIEKTISKAKGQPREPQVYTLPPSREEMTKNQVSL |
| | and X₄ is I | TCLVKGFYPSDIAVEWESNGQPENNYKTTPPVLDSDGSFFLYSKLTVDKS |
| | and the | RWQQGNVFSCSVMHEALHNHYTQKSLSLSPG |
| | remainder are | |
| | L or I. | |
| 60 | IL-2 N88D | APTSSSTKKTQLQLEHLLLDLQMILNGINNYKNPKLTRMLTFKFYMPKKA |
| | V69A, Q74P, | TEX₁KHX₂QCLEEELKPLEEALNLAPSKNFHX₃RPRDLISDINVIVLELKG |
| | C125S, | SETTFMCEYADETATIVEFX₄NRWITFSQSIISTLT |
| | wherein at | |
| | least one of | |
| | X₁, X₂, X₃, | |
| | and X₄ is I | |
| | and the | |
| | remainder are | |
| | L or I. | |

In some embodiments, the sequences shown in the table or throughout comprise one or more mutations that correspond to positions L53, L56, L80, and L118. In some embodiments, the sequences shown in the table or throughout do not comprise one or more mutations that correspond to positions L53, L56, L80, and L118. In some embodiments, the sequences shown in the table or throughout the present application comprise one or more mutations that correspond to positions L59I, L631, 124L, L94I, L96I or L132I or other substitutions at the same positions. In some embodiments, the sequences shown in the table or throughout the present application do not comprise one or more mutations that correspond to positions L59I, L631, 124L, L94I, L96I or L132I or other substitutions at the same positions. In some embodiments, the mutation is leucine to isoleucine. In some embodimnets, the mutein does not comprise other mutations than as shown or described herein. In some embodiments, the peptide comprises a sequence of SEQ ID NO: 21, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39, SEQ ID NO: 40, SEQ ID NO: 41, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, SEQ ID NO: 54, SEQ ID NO: 55, or SEQ ID NO: 56, SEQ ID NO: 57, SEQ ID NO: 58, SEQ ID NO: 59, or SEQ ID NO: 60.

In some embodiments, the protein comprises an IL-2 mutein as provided for herein. In some embodiments, a polypeptide is provided comprising SEQ ID NO: 59 or SEQ ID NO: 60, wherein at least one of X₁, X₂, X₃, and X₄ is I and the remainder are L or I. In some embodiments, X₁, X₂, and X₃ are L and X₄ is I. In some embodiments, X₁, X₂, and X₄ are L and X₃ is I. In some embodiments, X₂, X₃, and X₄ are L and X₁ is I. In some embodiments, X₁, X₃, and X₄ are L and X₂ is I. In some embodiments, X₁ and X₂ are L and X₃ and X₄ are I. In some embodiments, X₁ and X₃ are L and X₂ and X₄ are I. In some embodiments, X₁ and X₄ are L and X₂ and X₃ are I. In some embodiments, X₂ and X₃ are L and X₁ and X₄ are I. In some embodiments, X₂ and X₄ are L and X₁ and X₃ are I. In some embodiments, X₃ and X₄ are L and X₁ and X₂ are I. In some embodiments, X₁, X₂, and X₃ are L and X₄ is I. In some embodiments, X₂, X₃, and X₄ are L and X₁ is I. In some embodiments, X₁, X₃, and X₄ are L and X₂ is I. In some embodiments, X₁, X₂, and X₄ are L and X₃ is I.

In some embodiments, the Fc portion of the fusion is not included. In some embodiments, the peptide consists essentially of an IL-2 mutein provided for herein. In some embodiments, the protein is free of a Fc portion.

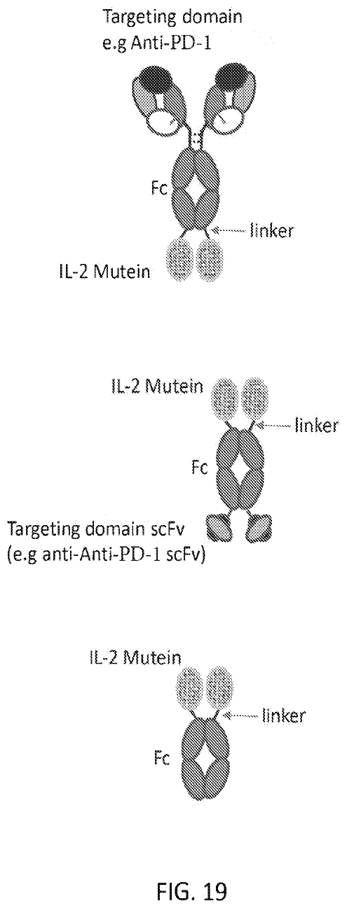

For illustrative purposes only, embodiments of an IL-2 mutein fused with a Fc and with a targeting moiety are illustrated in FIG. 19.

The sequences are for illustrative purposes only and are not intended to be limiting. In some embodiments, the compound comprises an amino acid sequence of SEQ ID NO: 53, 54, 55, or 56. In some embodiments, the compound comprises an amino acid sequence of SEQ ID NO: 53, 54, 55, or 56 with or without a C125A or C125S mutation. In some embodiments, the residue at position 125 is C, S, or A. In some embodiments, the compound comprises an amino acid sequence of SEQ ID NO: 59 or SEQ ID NO: 60, wherein at least one of X₁, X₂, X₃, and X₄ is I and the remainder are L or I. In some embodiments, the protein comprises an IL-2 mutein as provided for herein. In some embodiments, a polypeptide is provided comprising SEQ ID NO: 59 or SEQ ID NO: 60, wherein at least one of X₁, X₂, X₃, and X₄ is I and the remainder are L or I. In some embodiments, X₁, X₂, and X₃ are L and X₄ is I. In some embodiments, X₁, X₂, and X₄ are L and X₃ is I. In some embodiments, X₂, X₃, and X₄ are L and X₁ is I. In some embodiments, X₁, X₃, and X₄ are L and X₂ is I. In some embodiments, X₁ and X₂ are L and X₃ and X₄ are I. In some embodiments, X₁ and X₃ are L and X₂ and X₄ are I. In some embodiments, X₁ and X₄ are L and X₂ and X₃ are I. In some embodiments, X₂ and X₃ are L and X₁ and X₄ are I. In some embodiments, X₂ and X₄ are L and X₁ and X₃ are I. In some embodiments, X₃ and X₄ are L and X₁ and X₂ are I. In some embodiments, X₁, X₂, and X₃ are L and X₄ is I. In some embodiments, X₂, X₃, and X₄ are L and X₁ is I. In some embodiments, X₁, X₃, and X₄ are L and X₂ is I. In some embodiments, X₁, X₂, and X₄ are L and X₃ is I.

Each of the proteins may also be considered to have the C125S and the LALA and/or G237A mutations as provided for herein. The C125 substitution can also be C125A as described throughout the present application.

In an embodiment, an IL-2 mutein molecule comprises at least 60, 70, 80, 85, 90, 95, or 97% sequence identity or homology with a naturally occurring human IL-2 molecule, e.g., a naturally occurring IL-2 sequence disclosed herein or those that incorporated by reference.

As described herein, the IL-2 muteins can be part of a bi-specific molecule with a tethering moiety, such as a MAdCAM antibody that will target the IL-2 mutein to a MAdCAM expressing cell. As described herein, the bispecific molecule can be produced from two polypeptide chains. In some embodiments, the following can be used:

Table of MAdCAM-IL-2 Mutein Bispecific Compounds

| | Chain 1 N-terminal to C-terminal Molecule | | | | | |
|---|---|---|---|---|---|---|
| | Component Sequence IDs | | | | | |
| | | Antibody | | | Chain 2 N-terminal | |
| | | Heavy | | | to C-terminal | |
| | | Chain | | | Molecule Component | |
| | | CH1-CH2- | | C- | Sequence IDs | |
| | Antibody VH | CH3 | | terminal | Light Chain | Light Chain |
| Detail | Domain | Domains | Linker 1 | Moiety | VK Domain | CK Domain |
| 1. Anti- | Rat Anti- | SEQ ID | SEQ ID | SEQ ID | Rat Anti- | SEQ ID |
| MAdCam-Fc- | MAdCam-VH1 | NO: 44 | NO: 23 | NO: 35 | MAdCam-VK1 | NO: 45 |
| IL-2 N88D | | | | | | |
| V69A, Q74P | | | | | | |
| 2. Anti- | Rat Anti- | SEQ ID | SEQ ID | SEQ ID | Rat Anti- | SEQ ID |
| MAdCam- | MAdCam-VH2 | NO: 44 | NO: 23 | NO: 35 | MAdCam-VK2 | NO: 45 |
| Fc-IL-2 | | | | | | |
| N88D V69A, | | | | | | |
| Q74P | | | | | | |
| 3. Anti- | Rat Anti- | SEQ ID | SEQ ID | SEQ ID | Rat Anti- | SEQ ID |
| MAdCam- | MAdCam-VH1 | NO: 44 | NO: 23 | NO: 41 | MAdCam-VK3 | NO: 45 |
| Fc-IL-2 | | | | | | |
| L118I N88D | | | | | | |
| V69A, Q74P | | | | | | |
| 4. TTJ2- | Human TTJ2- | SEQ ID | SEQ ID | SEQ ID | Human TTJ2- | SEQ ID |
| Fc-IL-2 | VH | NO: 44 | NO: 23 | NO: 41 | VK | NO: 45 |
| L118I N88D | | | | | | |
| V69A, Q74P | | | | | | |
| 5. anti | Anti-MAdCam | SEQ ID | SEQ ID | SEQ ID | Anti-MAdCam | SEQ ID |
| hu.MAdCAM- | Human VH3 | NO: 44 | NO: 23 | NO: 41 | Human VK3 | NO: 45 |
| Fc-IL-2 | | | | | | |
| L118I N88D | | | | | | |
| V69A, Q74P | | | | | | |
| 6. anti | Anti-MAdCam | SEQ ID | SEQ ID | SEQ ID | Anti-MAdCam | SEQ ID |
| hu.MAdCAM- | Human VH4 | NO: 44 | NO: 23 | NO: 41 | Human VK4 | NO: 45 |
| Fc-IL-2 | | | | | | |
| L118I N88D | | | | | | |
| V69A, Q74P | | | | | | |
| 7. anti | Anti-MAdCam | SEQ ID | SEQ ID | SEQ ID | Anti-MAdCam | SEQ ID |
| hu.MAdCAM- | Human VH5 | NO: 44 | NO: 23 | NO: 41 | Human VK5 | NO: 45 |
| Fc-IL-2 | | | | | | |
| L118I N88D | | | | | | |
| V69A, Q74P | | | | | | |

The proteins can be produced with or without a C125A or C125S mutation in the IL-2 mutein. Examples of IL-2 muteins that can be included are illustrated herein, such as, but not limited to, a sequence of SEQ ID NO: 59 or SEQ ID NO: 60.

In some embodiments, the constant kappa domain in any of the light chains can be replaced with a constant lambda domain.

### Auto-Immune Disorders

In some embodiments, methods of treating a subject having Type 1 diabetes are provided. In some embodiments, methods of treating a subject having Type 1 diabetes or at risk for developing Type 1 diabetes are provided. In some embodiments, methods of treating a subject at risk for having Type 1 diabetes are provided. In some embodiments, methods of treating a subject at elevated risk for having Type 1 diabetes are provided.

In some embodiments, the methods comprise administering a polypeptide, protein, antibody, or pharmaceutical composition as provided for herein to the subject to treat or prevent Type 1 diabetes in the subject. In some embodiments, the composition comprises an effector domain linked to an antibody that binds to MadCAM. In some embodiments, the effector domain is an IL-2 mutein. In some embodiments, the composition comprises a bi-specific molecule that comprises a portion that binds to MAdCAM and another portion that binds to PD-1. In some embodiments, the molecule that binds to PD-1 is a PD-1 agonist. In some embodiments, the portions that bind to MAdCAM and/or PD-1 are antibodies. In some embodiments, the format is an IgG format or a scFv format.

In some embodiments, methods of delaying the onset of Type 1 diabetes in a subject are provided. In some embodiments, the methods comprise administering a polypeptide, protein, antibody, or pharmaceutical composition as provided for herein to the subject to delay the onset of Type 1 diabetes in the subject. In some embodiments, the composition comprises a bi-specific molecule that comprises a portion that binds to MAdCAM and another portion that binds to PD-1. In some embodiments, the molecule that binds to PD-1 is a PD-1 agonist. In some embodiments, the portions that bind to MAdCAM and/or PD-1 are antibodies. In some embodiments, the format is an IgG format or a scFv format. In some embodiments, the subject is at risk for developing Type 1 diabetes.

In some embodiments, methods of delaying, reducing, treating, or preventing hyperglycemia in a subject are provided. In some embodiments, the methods comprise administering a polypeptide, protein, antibody, or pharmaceutical composition as provided for herein to the subject to delay, reduce, treat, or prevent hyperglycemia in the subject. In some embodiments, the composition comprises a bi-specific molecule that comprises a portion that binds to MAdCAM and another portion that binds to PD-1. In some embodiments, the molecule that binds to PD-1 is a PD-1 agonist. In some embodiments, the portions that bind to MAdCAM and/or PD-1 are antibodies. In some embodiments, the format is an IgG format or a scFv format. In some embodiments, the subject is a subject having, at risk, or elevated risk, for having Type 1 diabetes or hyperglycemia.

As provided for herein, in some embodiments the PD-1 agonist is replaced with an IL-2 mutein, such as those provided for herein.

### Therapeutic Compounds

A therapeutic compound comprises a specific targeting moiety functionally associated with an effector binding/modulating moiety. The targeting moiety can be one that, for example, binds to MAdCAM. In some embodiments, the effector moiety is a PD-1 agonist or an IL-2 mutein. In some embodiments, the specific targeting moiety and effector binding/modulating moiety are linked to one another by a covalent or noncovalent bond, e.g., a covalent or non-covalent bond directly linking the one to the other. In other embodiments, a specific targeting moiety and effector binding/modulating moiety are linked, e.g., covalently or noncovalently, through a linker moiety. E.g., in the case of a fusion polypeptide, a polypeptide sequence comprising the specific targeting moiety and a polypeptide sequence can be directly linked to one another or linked through one or more linker sequences. In some embodiments, the linker moiety comprises a polypeptide. Linkers are not, however, limited to polypeptides. In some embodiments, a linker moiety comprises other backbones, e.g., a non-peptide polymer, e.g., a PEG polymer. In some embodiments, a linker moiety can comprise a particle, e.g., a nanoparticle, e.g., a polymeric nanoparticle. In some embodiments, a linker moiety can comprise a branched molecule, or a dendrimer. However, in embodiments where the effector binding/modulating moiety comprises an ICIM binding/modulating moiety (which binds an effector like PD-1) structures that result in clustering in the absence of target binding should be avoided as they may cause clustering in the absence of target binding. Thus in embodiments, the therapeutic compound has a structure, e.g., the copies of an ICIM are sufficiently limited, such that clustering in the absence of target binding is minimized or substantially eliminated, or eliminated, or is sufficiently minimized that substantial systemic immune suppression does not occur.

In some embodiments, a therapeutic compound comprises a polypeptide comprising a specific targeting moiety covalently or non-covalently conjugated to an effector binding/modulating moiety. In some embodiments, a therapeutic molecule comprises a fusion protein having comprising a specific targeting moiety fused, e.g., directly or through a linking moiety comprising one or more amino acid residues, to an effector binding/modulating moiety. In some embodiments, a therapeutic molecule comprises a polypeptide comprising a specific targeting moiety linked by a non-covalent bond or a covalent bond, e.g., a covalent bond other than a peptide bond, e.g., a sulfhydryl bond, to an effector binding/modulating moiety.

In some embodiments, a therapeutic compound comprises polypeptide, e.g., a fusion polypeptide, comprising:
1.a) a specific targeting moiety comprising a target specific binding polypeptide;
1.b) a specific targeting moiety comprising a target ligand binding molecule;
1.c) a specific targeting moiety comprising an antibody molecule;
1.d) a specific targeting moiety comprising a single chain antibody molecule, e.g., a scFv domain; or
1.e) a specific targeting moiety comprising a first of the light or heavy chain variable region of an antibody molecule, and wherein the other variable region is covalently or non-covalently associated with the first;
and
2.a) an effector binding/modulating moiety comprising an effector specific binding polypeptide;
2.b) an effector binding/modulating moiety comprising an effector ligand binding molecule;
2.c) an effector binding/modulating moiety comprising an antibody molecule;
2.d) an effector binding/modulating moiety comprising a single chain antibody molecule, e.g., a scFv domain; or
2.e) an effector binding/modulating moiety comprising a first of the light or heavy chain variable region of an antibody molecule, and wherein the other variable region is covalently or non-covalently associated with the first.

In some embodiments, a therapeutic compound comprises 1.a and 2.a.

In some embodiments, a therapeutic compound comprises 1.a and 2.b.

In some embodiments, a therapeutic compound comprises 1.a and 2.c.

In some embodiments, a therapeutic compound comprises 1.a and 2.d.

In some embodiments, a therapeutic compound comprises 1.a and 2.e.

In some embodiments, a therapeutic compound comprises 1.b and 2.a.

In some embodiments, a therapeutic compound comprises 1.b and 2.b.

In some embodiments, a therapeutic compound comprises 1.b and 2.c.

In some embodiments, a therapeutic compound comprises 1.b and 2.d.

In some embodiments, a therapeutic compound comprises 1.b and 2.e.

In some embodiments, a therapeutic compound comprises 1.c and 2.a.

In some embodiments, a therapeutic compound comprises 1.c and 2.b.

In some embodiments, a therapeutic compound comprises 1.c and 2.c.

In some embodiments, a therapeutic compound comprises 1.c and 2.d.

In some embodiments, a therapeutic compound comprises 1.c and 2.e.

In some embodiments, a therapeutic compound comprises 1.d and 2.a.

In some embodiments, a therapeutic compound comprises 1.d and 2.b.

In some embodiments, a therapeutic compound comprises 1.d and 2.c.

In some embodiments, a therapeutic compound comprises 1.d and 2.d.

In some embodiments, a therapeutic compound comprises 1.d and 2.e.

In some embodiments, a therapeutic compound comprises 1.e and 2.a.

In some embodiments, a therapeutic compound comprises 1.e and 2.b.

In some embodiments, a therapeutic compound comprises 1.e and 2.c.

In some embodiments, a therapeutic compound comprises 1.e and 2.d.

In some embodiments, a therapeutic compound comprises 1.e and 2.e.

Therapeutic compounds disclosed herein can, for example, comprise a plurality of effector binding/modulating and specific targeting moieties. Any suitable linker or platform can be used to present the plurality of moieties. The linker is typically coupled or fused to one or more effector binding/modulating and targeting moieties.

In some embodiments, two (or more) linkers associate, either covalently or non-covalently, e.g., to form a hetero or homo-dimeric therapeutic compound. E.g., the linker can comprise an Fc region and two Fc regions associate with one another. In some embodiments of a therapeutic compound comprising two linker regions, the linker regions can self-associate, e.g., as two identical Fc regions. In some embodiments of a therapeutic compound comprising two linker regions, the linker regions are not capable of, or not capable of substantial, self-association, e.g., the two Fc regions can be members of a knob and hole pair.

Non-limiting exemplary configurations of therapeutic compounds comprise the following (e.g., in N to C terminal order):
R1-Linker Region A-R2
R3-Linker Region B-R4,
   wherein,
R1, R2, R3, and R4, each independently comprises an effector binding/modulating moiety, e.g., an ICIM binding/modulating moiety, an IIC binding/modulating moiety, ICSM binding/modulating moiety, or an SM binding/modulating moiety; a specific targeting moiety; or is absent, provided that at least one of R1 and R2 is not absent, and at least one of R3 and R4 is not absent;
Linker Region A and Linker B comprise moieties that can associate with one another, e.g., Linker A and Linker B each comprises an Fc moiety provided that an effector binding/modulating moiety and a specific targeting moiety are present.

In some embodiments:
R1 comprises an effector binding/modulating moiety, e.g., an ICIM binding/modulating moiety, an IIC binding/modulating moiety, ICSM binding/modulating moiety, or an SM binding/modulating moiety, or is absent;
R2 comprises a specific targeting moiety, or is absent;
R3 comprises an effector binding/modulating moiety, e.g., an ICIM binding/modulating moiety, an IIC binding/modulating moiety, ICSM binding/modulating moiety, or an SM binding/modulating moiety, or is absent;
R4 comprises a specific targeting moiety, or is absent;
Linker Region A and Linker B comprise moieties that can associate with one another, e.g., Linker A and Linker B each comprises an Fc moiety, provided that one of R1 or R3 is present and one of R2 or R4 is present.

In some embodiments:
R1 comprises a specific targeting moiety, or is absent;
R2 comprises an effector binding/modulating moiety, e.g., an ICIM binding/modulating moiety, an IIC binding/modulating moiety, ICSM binding/modulating moiety, or an SM binding/modulating moiety, or is absent;
R3 comprises a specific targeting moiety, or is absent;
R4 comprises an effector binding/modulating moiety, e.g., an ICIM binding/modulating moiety, an IIC binding/modulating moiety, ICSM binding/modulating moiety, or an SM binding/modulating moiety, or is absent;
Linker Region A and Linker B comprise moieties that can associate with one another, e.g., Linker A and Linker B each comprises an Fc moiety, provided that one of R1 or R3 is present and one of R2 or R4 is present.

Non-limiting examples include, but are not limited to:

| | Linker | | | Linker | | |
|---|---|---|---|---|---|---|
| R1 | Region A | R2 | R3 | Region B | R4 | Other |
| HCVR and | Fc Region | scFv | HCVR | Fc Region | scFv | Self-Pairing |
| LCVR | | | and | | | Linker Regions |
| | | | LCVR | | | |
| HCVR and | Fc Region | scFv | HCVR | Fc Region | scFv | Non-Self |
| LCVR | | | and | | | Pairing linker |
| | | | LCVR | | | regions |
| HCVR and | Fc Region | scFv | HCVR | Fc Region | scFv | Self-Pairing |
| LCVR (or | | | and | | | Linker Regions |
| absent) | | | LCVR | | | One of R1 or |
| | | | (or | | | R3 is absent. |
| | | | absent) | | | |
| HCVR and | Fc Region | scFv | HCVR | Fc Region | scFv | Non-Self |
| LCVR (or | | | and | | | Pairing Linker |
| absent) | | | LCVR | | | Regions |
| | | | (or | | | One of R1 or |
| | | | absent) | | | R3 is absent. |
| HCVR and | Fc Region | scFv (or | HCVR | Fc Region | scFv (or | Self-Pairing |
| LCVR | | absent) | and | | absent) | linker regions |
| | | | LCVR | | | One of R2 or |
| | | | | | | R4 is absent. |
| HCVR and | Fc Region | scFv (or | HCVR | Fc Region | scFv (or | Non-Self |
| LCVR | | absent) | and | | absent) | Pairing linker |
| | | | LCVR | | | regions |
| | | | | | | One of R2 or |
| | | | | | | R4 is absent. |
| HCVR and | Fc Region | scFv | HCVR | Fc Region | scFv | Self-Pairing |
| LCVR | | | and | | | Linker Regions |
| | | | LCVR | | | R1 and R3 are |
| | | | | | | the same |
| HCVR and | Fc Region | scFv | HCVR | Fc Region | scFv | Non-Self |
| LCVR | | | and | | | Pairing linker |
| | | | LCVR | | | regions |
| | | | | | | R1and R3 are |
| | | | | | | different |
| HCVR and | Fc Region | scFv | HCVR | Fc Region | scFv | Self-Pairing |
| LCVR | | | and | | | Linker Regions |
| | | | LCVR | | | R2 and R4 are |
| | | | | | | the same |
| HCVR and | Fc Region | scFv | HCVR | Fc Region | scFv | Non-Self |
| LCVR | | | and | | | Pairing linker |
| | | | LCVR | | | regions |
| | | | | | | R2and R4 are |
| | | | | | | different |

HCVR and LCVR: refers to a moiety comprising an antigen binding portion of a heavy and light chain variable region, typically with the heavy chain fused to the Linker region.
Self-pairing: wherein a liker region can pair with itself, e.g., an Fc region that can pair a copy of itself.
Non-Self Pairing: wherein a Linker Region does not pair with itself, or does not substantially pair with itself, e.g., an Fc region does not or does not significantly pair with itself, e.g., wherein Linker Region A and Linker Region B are members of a knob and hole pair.

In some embodiments:
R1, R2, R3 and R4 each independently comprise: an effector binding modulating moiety that activates an inhibitory receptor on an immune cell, e.g., a T cell or a B cell, e.g., a PD-L1 molecule or a functional anti-PD-1 antibody molecule (an agonist of PD-1); a specific targeting moiety; or is absent, provided that at least one of R1 and R2 is not absent, and at least one of R3 and R4 is not absent;
provided that an effector binding moiety and a specific targeting moiety are present.

In some embodiments, Linker A and Linker B comprise Fc moieties (e.g., self-pairing Fc moieties).

In some embodiment:
R1 and R3 independently comprise an effector binding modulating moiety that activates an inhibitory receptor on an immune cell, e.g., a T cell or a B cell, e.g., a PD-L1 molecule or a functional anti-PD-1 antibody molecule (an agonist of PD-1); and
R2 and R4 independently comprise specific targeting moieties, e.g., scFv molecules against a tissue antigen.

In some embodiments, Linker A and Linker B comprise Fc moieties (e.g., self-pairing Fc moieties).

In some embodiments:
R1 and R3 independently comprise a functional anti-PD-1 antibody molecule (an agonist of PD-1); and
R2 and R4 independently comprise specific targeting moieties, e.g., scFv molecules against a tissue antigen.

In some embodiments, Linker A and Linker B comprise Fc moieties (e.g., self-pairing Fc moieties).

In some embodiments:
R1 and R3 independently comprise specific targeting moieties, e.g., an anti-tissue antigen antibody; and
R2 and R4 independently comprise a functional anti-PD-1 antibody molecule (an agonist of PD-1), e.g., an scFv molecule.

In some embodiments, Linker A and Linker B comprise Fc moieties (e.g., self-pairing Fc moieties).

In some embodiment:
R1 and R3 independently comprise a PD-L1 molecule (an agonist of PD-1); and
R2 and R4 independently comprise specific targeting moieties, e.g., scFv molecules against a tissue antigen; and

In some embodiments, Linker A and Linker B comprise Fc moieties (e.g., self-pairing Fc moieties).

In some embodiment:
R1 and R3 independently comprise specific targeting moieties, e.g., an anti-tissue antigen antibody; and
R2 and R4 independently comprise a PD-L1 molecule (an agonist of PD-1).

In some embodiments, Linker A and Linker B comprise Fc moieties (e.g., self-pairing Fc moieties).

In some embodiment:
R1, R2, R3 and R4 each independently comprise: an SM binding/modulating moiety which modulates, e.g., binds and inhibits, sequesters, degrades or otherwise neutralizes a substance, e.g., a soluble molecule that modulates an immune response, e.g., PD-1 agonist or IL-2 mutein, or is absent, provided that at least one of R1 and R2 is not absent, and at least one of R3 and R4 is not absent;
provided that an SM binding/modulating moiety and a specific targeting moiety are present.

In some embodiments, Linker A and Linker B comprise Fc moieties (e.g., self-pairing Fc moieties or Fc moieties that do not, or do not substantially self-pair).

In some embodiment:
R1 and R3 independently comprise an SM binding/modulating moiety which modulates, e.g., binds and inhibits, sequesters, degrades or otherwise neutralizes a substance, e.g., PD-1 agonist or IL-2 mutein; and
R2 and R4 independently comprise specific targeting moieties, e.g., scFv molecules against a tissue antigen, such as, MAdCAM.

In some embodiments, Linker A and Linker B comprise Fc moieties (e.g., self-pairing Fc moieties or Fc moieties that do not, or do not substantially self-pair).

In some embodiment:
R1 and R3 independently comprise a PD-1 agonist, IL-2 mutein, CD39 molecule or a CD73 molecule; and
R2 and R4 independently comprise specific targeting moieties, e.g., scFv molecules against a tissue antigen, such as, MAdCAM.

In some embodiments, Linker A and Linker B comprise Fc moieties (e.g., self-pairing Fc moieties or Fc moieties that do not, or do not substantially self-pair).

In some embodiment:
R1 and R3 each comprises a PD-1 agonist, IL-2 mutein, or CD39 molecule; and
R2 and R4 independently comprise specific targeting moieties, e.g., scFv molecules against a tissue antigen, such as, MAdCAM.

In some embodiments, Linker A and Linker B comprise Fc moieties (e.g., self-pairing Fc moieties or Fc moieties that do not, or do not substantially self-pair).

In an embodiment:
R1, R2, R3 and R4 each independently comprise: an IL-2 mutein molecule; a specific targeting moiety; or is absent, provided that at least one of R1 and R2 is not absent, and at least one of R3 and R4 is not absent;
provided that an IL-2 mutein molecule and a specific targeting moiety (e.g., one that binds to MAdCAM) are present.

In an embodiment Linker A and Linker B comprise Fc moieties (e.g., self-pairing Fc moieties or Fc moieties that do not, or do not substantially self-pair). As provided for herein and throughout, in some embodiments, the targeting moiety is an anti-MAdCAM antibody.

In an embodiment:
R1 and R3 each comprise an IL-2 mutein molecule; and
R2 and R4 independently comprise specific targeting moieties, e.g., scFv molecules against a tissue antigen.

In an embodiment Linker A and Linker B comprise Fc moieties (e.g., self-pairing Fc moieties or Fc moieties that do not, or do not substantially self-pair).

### Linker Regions

As discussed elsewhere herein specific targeting and effector binding/modulating moieties can be linked by linker regions. Any linker region described herein can be used as a linker. For example, linker Regions A and B can comprise Fc regions. In some embodiments, a therapeutic compound comprises a Linker Region that can self-associate. In some embodiments, a therapeutic compound comprises a Linker Region that has a moiety that minimizes self-association, and typically Linker Region A and Linker Region B are heterodimers. Linkers also include glycine/serine linkers. In some embodiments, the linker can comprise one or more repeats of GGGGS (SEQ ID NO: 23). In some embodiments, the linker comprises 1, 2, 3, 4, or 5 repeats of SEQ ID NO: 23 (repeats disclosed as SEQ ID NO: 1549). In some embodiments, the linker comprises or GGGGSGGGGSGGGGSGGGGS (SEQ ID NO: 22) GGGGSGGGGSGGGGS (SEQ ID NO: 30). These linkers can be used in any of the therapeutic compounds or compositions provided herein.

The linker region can comprise a Fc region that has been modified (e.g. mutated) to produce a heterodimer. In some embodiments, the CH3 domain of the Fc region can be mutated. Examples of such Fc regions can be found in, for example, U.S. Pat. No. 9,574,010, which is hereby incorporated by reference in its entirety. The Fc region as defined herein comprises a CH3 domain or fragment thereof, and may additionally comprise one or more addition constant region domains, or fragments thereof, including hinge, CH1, or CH2. It will be understood that the numbering of the Fc amino acid residues is that of the EU index as in Kabat et al., 1991, NIH Publication 91-3242, National Technical Information Service, Springfield, Va. The “EU index as set forth in Kabat” refers to the EU index numbering of the human IgG1 Kabat antibody. For convenience, Table B of U.S. Pat. No. 9,574,010 provides the amino acids numbered according to the EU index as set forth in Kabat of the CH2 and CH3 domain from human IgG1, which is hereby incorporated by reference. Table 1.1 of U.S. Pat. No. 9,574,010 provides mutations of variant Fc heterodimers that can be used as linker regions. Table 1.1 of U.S. Pat. No. 9,574,010 is hereby incorporated by reference.

In some embodiments, the Linker Region A comprises a first CH3 domain polypeptide and a the Linker Region B comprises a second CH3 domain polypeptide, the first and second CH3 domain polypeptides independently comprising amino acid modifications as compared to a wild-type CH3 domain polypeptide, wherein the first CH3 domain polypeptide comprises amino acid modifications at positions T350, L351, F405, and Y407, and the second CH3 domain polypeptide comprises amino acid modifications at positions T350, T366, K392 and T394, wherein the amino acid modification at position T350 is T350V, T3501, T350L or T350M; the amino acid modification at position L351 is L351Y; the amino acid modification at position F405 is F405A, F405V, F405T or F405S; the amino acid modification at position Y407 is Y407V, Y407A or Y407I; the amino acid modification at position T366 is T366L, T366I, T366V, or T366M, the amino acid modification at position K392 is K392F, K392L or K392M, and the amino acid modification at position T394 is T394W, and wherein the numbering of amino acid residues is according to the EU index as set forth in Kabat.

In some embodiments, the amino acid modification at position K392 is K392M or K392L. In some embodiments, the amino acid modification at position T350 is T350V. In some embodiments, the first CH3 domain polypeptide further comprises one or more amino acid modifications selected from Q347R and one of S400R or S400E. In some embodiments, the second CH3 domain polypeptide further comprises one or more amino acid modifications selected from L351Y, K360E, and one of N390R, N390D or N390E. In some embodiments, the first CH3 domain polypeptide further comprises one or more amino acid modifications selected from Q347R and one of S400R or S400E, and the second CH3 domain polypeptide further comprises one or more amino acid modifications selected from L351Y, K360E, and one of N390R, N390D or N390E. In some embodiments, the amino acid modification at position T350 is T350V. In some embodiments, the amino acid modification at position F405 is F405A. In some embodiments, the amino acid modification at position Y407 is Y407V. In some embodiments, the amino acid modification at position T366 is T366L or T366I. In some embodiments, the amino acid modification at position F405 is F405A, the amino acid modification at position Y407 is and Y407V, the amino acid modification at position T366 is T366L or T366I, and the amino acid modification at position K392 is K392M or K392L. In some embodiments, the first CH3 domain polypeptide comprises the amino acid modifications T350V, L351Y, S400E, F405V and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405T and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405S and Y407V, and the second CH3 domain polypeptide comprises the amino acid modifications T350V, T366L, N390R, K392M and T394W. In some embodiments, the first CH3 domain polypeptide comprises the amino acid modifications T350V, L351Y, S400E, F405A and Y407V, and the second CH3 domain polypeptide comprises the amino acid modifications T350V, L351Y, T366L, N390R, K392M and T394W. In some embodiments, the first CH3 domain polypeptide comprises the amino acid modifications Q347R, T350V, L351Y, S400E, F405A and Y407V, and the second CH3 domain polypeptide comprises the amino acid modifications T350V, K360E, T366L, N390R, K392M and T394W. In some embodiments, the first CH3 domain polypeptide comprises the amino acid modifications T350V, L351Y, S400R, F405A and Y407V, and the second CH3 domain polypeptide comprises the amino acid modifications T350V, T366L, N390D, K392M and T394W. In some embodiments, the first CH3 domain polypeptide comprises the amino acid modifications T350V, L351Y, S400R, F405A and Y407V, and the second CH3 domain polypeptide comprises the amino acid modifications T350V, T366L, N390E, K392M and T394W. In some embodiments, the first CH3 domain polypeptide comprises the amino acid modifications T350V, L351Y, S400E, F405A and Y407V, and the second CH3 domain polypeptide comprises the amino acid modifications T350V, T366L, N390R, K392L and T394W. In some embodiments, the first CH3 domain polypeptide comprises the amino acid modifications T350V, L351Y, S400E, F405A and Y407V, and the second CH3 domain polypeptide comprises the amino acid modifications T350V, T366L, N390R, K392F and T394W.

In some embodiments, an isolated heteromultimer comprising a heterodimeric CH3 domain comprising a first CH3 domain polypeptide and a second CH3 domain polypeptide, the first CH3 domain polypeptide comprising amino acid modifications at positions F405 and Y407, and the second CH3 domain polypeptide comprising amino acid modifications at positions T366 and T394, wherein: (i) the first CH3 domain polypeptide further comprises an amino acid modification at position L351, and (ii) the second CH3 domain polypeptide further comprises an amino acid modification at position K392, wherein the amino acid modification at position F405 is F405A, F405T, F405S or F405V; and the amino acid modification at position Y407 is Y407V, Y407A, Y407L or Y407I; the amino acid modification at position T394 is T394W; the amino acid modification at position L351 is L351Y; the amino acid modification at position K392 is K392L, K392M, K392V or K392F, and the amino acid modification at position T366 is T366I, T366L, T366M or T366V, wherein the heterodimeric CH3 domain has a melting temperature (Tm) of about 70.degree. C. or greater and a purity greater than about 90%, and wherein the numbering of amino acid residues is according to the EU index as set forth in Kabat.

In some embodiments, the Linker Region A comprises a first CH3 domain polypeptide and a t Linker Region B comprises a second CH3 domain polypeptide, wherein the first CH3 domain polypeptide comprising amino acid modifications at positions F405 and Y407, and the second CH3 domain polypeptide comprising amino acid modifications at positions T366 and T394, wherein: (i) the first CH3 domain polypeptide further comprises an amino acid modification at position L351, and (ii) the second CH3 domain polypeptide further comprises an amino acid modification at position K392, wherein the amino acid modification at position F405 is F405A, F405T, F405S or F405V; and the amino acid modification at position Y407 is Y407V, Y407A, Y407L or Y407I; the amino acid modification at position T394 is T394W; the amino acid modification at position L351 is L351Y; the amino acid modification at position K392 is K392L, K392M, K392V or K392F, and the amino acid modification at position T366 is T366I, T366L, T366M or T366V, wherein the heterodimeric CH3 domain has a melting temperature (Tm) of about 70 C. or greater and a purity greater than about 90%, and wherein the numbering of amino acid residues is according to the EU index as set forth in Kabat. In some embodiments, the amino acid modification at position F405 is F405A. In some embodiments, the amino acid modification at position T366 is T366I or T366L. In some embodiments, the amino acid modification at position Y407 is Y407V. In some embodiments, the amino acid modification at position F405 is F405A, the amino acid modification at position Y407 is Y407V, the amino acid modification at position T366 is T366I or T366L, and the amino acid modification at position K392 is K392L or K392M. In some embodiments, the amino acid modification at position F405 is F405A, the amino acid modification at position Y407 is Y407V, the amino acid modification at position T366 is T366L, and the amino acid modification at position K392 is K392M. In some embodiments, the amino acid modification at position F405 is F405A, the amino acid modification at position Y407 is Y407V, the amino acid modification at position T366 is T366L, and the amino acid modification at position K392 is K392L. In some embodiments, the amino acid modification at position F405 is F405A, the amino acid modification at position Y407 is Y407V, the amino acid modification at position T366 is T366I, and the amino acid modification at position K392 is K392M. In some embodiments, the amino acid modification at position F405 is F405A, the amino acid modification at position Y407 is Y407V, the amino acid modification at position T366 is T366I, and the amino acid modification at position K392 is K392L. In some embodiments, the first CH3 domain polypeptide further comprises an amino acid modification at position S400 selected from S400D and S400E, and the second CH3 domain polypeptide further comprises the amino acid modification N390R. In some embodiments, the amino acid modification at position F405 is F405A, the amino acid modification at position Y407 is Y405V, the amino acid modification at position 5400 is S400E, the amino acid modification at position T366 is T366L, and the amino acid modification at position K392 is K392M.

In some embodiments, the modified first and second CH3 domains are comprised by an Fc construct based on a type G immunoglobulin (IgG). The IgG can be an IgG1, IgG2, IgG3 or IgG4.

Other Linker Region A and Linger Region B comprising variant CH3 domains are described in U.S. Pat. Nos. 9,499,634 and 9,562,109, each of which is incorporated by reference in its entirety.

A Linker Region A and Linker Region B can be complementary fragments of a protein, e.g., a naturally occurring protein such as human serum albumin. In embodiments, one of Linker Region A and Linker Region B comprises a first, e.g., an N terminal fragment of the protein, e.g., hSA, and the other comprises a second, e.g., a C terminal fragment of the protein, e.g., has. In an embodiment the fragments comprise an N terminal and a C terminal fragment. In an embodiment the fragments comprise two internal fragments. Typically the fragments do not overlap. In an embodiment the First and second fragment, together, provide the entire sequence of the original protein, e.g., hSA. The first fragment provides a N terminus and a C terminus for linking, e.g., fusing, to other sequences, e.g., sequences of R1, R2, R3, or R4 (as defined herein).

The Linker Region A and the Linker Region B can be derived from albumin polypeptide. In some embodiments, the albumin polypeptide is selected from native human serum albumin polypeptide and human alloalbumin polypeptide. The albumin polypeptide can be modified such that the Linker Region A and Linker Region B interact with one another to form heterodimers. Examples of modified albumin polypeptides are described in U.S. Pat. Nos. 9,388,231 and 9,499,605, each of which is hereby incorporated by reference in its entirety. Accordingly, provided herein are multifunctional heteromultimer proteins of the formula R1 Linker Region A-R2 and R3-Linker Region B-R4, wherein the Linker Region A and Linker Region B form a heteromultimer. In some embodiments, the Linker Region A comprises a first polypeptide and the Linker Region B comprises a second polypeptide; wherein each of said first and second polypeptides comprises an amino acid sequence comprising a segment of an albumin polypeptide selected from native human serum albumin polypeptide and human alloalbumin polypeptide; wherein said first and second polypeptides are obtained by segmentation of said albumin polypeptide at a segmentation site, such that the segmentation results in a deletion of zero to 3 amino acid residues at the segmentation site; wherein said first polypeptide comprises at least one mutation selected from A194C, L198C, W214C, A217C, L331C and A335C, and said second polypeptide comprises at least one mutation selected from L331C, A335C, V343C, L346C, A350C, V455C, and N458C; and wherein said first and second polypeptides self-assemble to form a quasi-native structure of the monomeric form of the albumin polypeptide.

In some embodiments, the segmentation site resides on a loop of the albumin polypeptide that has a high solvent accessible surface area (SASA) and limited contact with the rest of the albumin structure, b) the segmentation results in a complementary interface between the transporter polypeptides. These segmentation sites are described, for example, in U.S. Pat. No. 9,388,231, which is hereby incorporated by reference in its entirety.

In some embodiments, the first polypeptide comprises residues 1-337 or residues 1-293 of the albumin polypeptide with one or more of the mutations described herein. In some embodiments, the second polypeptide comprises residues of 342-585 or 304-585 of the albumin polypeptide with one or more of the mutations described herein. In some embodiments, the first polypeptide comprises residues 1-339, 1-300, 1-364, 1-441, 1-83, 1-171, 1-281, 1-293, 1-114, 1-337, or 1-336 of the albumin protein. In some embodiments, the second polypeptide comprises residues 301-585, 365-585, 442-585, 85-585, 172-585, 282-585, or 115-585, 304-585, 340-585, or 342-585 of the albumin protein.

In some embodiments, the first and second polypeptide comprise the residues of the albumin protein as shown in the table below. The sequence of the albumin protein is described below.

| First Polypeptide Residues | Second Polypeptide Residues |
|---|---|
| 1-300 | 301-585 |
| 1-364 | 365-585 |
| 1-441 | 442-585 |
| 1-83 | 85-585 |
| 1-171 | 172-585 |
| 1-281 | 282-585 |
| 1-114 | 115-585 |
| 1-339 | 340-585 |
| 1-337 | 342-585 |
| 1-293 | 304-585 |
| 1-336 | 342-585 |

In some embodiments, the first and second polypeptides comprise a linker that can form a covalent bond with one another, such as a disulfide bond. A non-limiting example of the linker is a peptide linker. In some embodiments, the peptide linker comprises GGGGS (SEQ ID NO: 23). The linker can be fused to the C-terminus of the first polypeptide and the N-terminus of the second polypeptide. The linker can also be used to attach the moieties described herein without abrogating the ability of the linkers to form a disulfide bond. In some embodiments, the first and second polypeptides do not comprise a linker that can form a covalent bond. In some embodiments, the first and second polypeptides have the following substitutions.

| First Polypeptide Substitution | Second Polypeptide Substitution |
|---|---|
| A217C | V343C |
| L331C | A350C |
| A217C | L346C |
| W214C | V343C |
| A335C | L346C |
| L198C | V455C |
| A217C | A335C |
| A217C | L331C |
| L198C | N458C |
| A194C | V455C |

The sequence of the albumin polypeptide can be The sequence of human albumin is as shown, in the post-protein form with the N-terminal signaling residues removed

(MKWVTFISLLFLFSSAYSRGVFRR SEQ ID NO: 1533)
(human albumin, SEQ ID NO: 42)
DAHKSEVAHRFKDLGEENFKALVLIAFAQYLQQCPFEDHVKLVN
EVTEFAKTCVADESAENCDKSLHTLFGDKLCTVATLRETYGEMA
DCCAKQEPERNECFLQHKDDNPNLPRLVRPEVDVMCTAFHDNEE
TFLKKYLYEIARRHPYFYAPELLFFAKRYKAAFTECCQAADKAA
CLLPKLDELRDEGKASSAKQRLKCASLQKFGERAFKAWAVARLS
QRFPKAEFAEVSKLVTDLTKVHTECCHGDLLECADDRADLAKYI
CENQDSISSKLKECCEKPLLEKSHCIAEVENDEMPADLPSLAAD
FVESKDVCKNYAEAKDVFLGMFLYEYARRHPDYSVVLLLRLAKT
YETTLEKCCAAADPHECYAKVFDEFKPLVEEPQNLIKQNCELFE
QLGEYKFQNALLVRYTKKVPQVSTPTLVEVSRNLGKVGSKCCKH
PEAKRMPCAEDYLSVVLNQLCVLHEKTPVSDRVTKCCTESLVNR
RPCFSALEVDETYVPKEFNAETFTFHADICTLSEKERQIKKQTA
LVELVKHKPKATKEQLKAVMDDFAAFVEKCCKADDKETCFAEEG
KKLVAASQAALGL.

In some embodiments, the Linker Region A and the Linker Region B form a heterodimer as described herein.

In some embodiments, the polypeptide comprises at the N-terminus an antibody comprised of F(ab′)2 on an IgG1 Fc backbone fused with scFvs on the C-terminus of the IgG Fc backbone. In some embodiments, the IgG Fc backbone is a IgG1 Fc backbone. In some embodiments, the IgG1 backbone is replaced with a IgG4 backbone, IgG2 backbone, or other similar IgG backbone. The IgG backbones described in this paragraph can be used throughout this application where a Fc region is referred to as part of the therapeutic compound. Thus, in some embodiments, the antibody comprised of F(ab′)2 on an IgG1 Fc backbone can be an anti-MAdCAM antibody or an anti-PD-1 antibody on an IgG1 Fc or any other targeting moiety or effector binding/modulating moiety provided herein. In some embodiments, the scFV segments fused to the C-terminus could be an anti-PD-1 antibody, if the N-terminus region is an anti-MAdCAM antibody, or anti-MAdCAM antibody, if the N-terminus region is an anti-PD-1 antibody. In this non-limiting example, the N-terminus can be the targeting moiety, such as any one of the ones provided for herein, and the C-terminus can be the effector binding/modulating moiety, such as any of the ones provided for herein. Alternatively, in some embodiments, the N-terminus can be the effector binding/modulating moiety, such as any one of the ones provided for herein, and the C-terminus can be the targeting moiety, such as any of the ones provided for herein.

In some embodiments, the N-terminus can be the targeting moiety, such as any one of the ones provided for herein, and the C-terminus can be the effector binding/modulating moiety, such as any of the ones provided for herein.

In some embodiments, the therapeutic compound comprises two polypeptides that homodimerize. In some embodiments, the N-terminus of the polypeptide comprises an effector binding/modulating moiety that is fused to a human IgG1 Fe domain (e.g. CH2 and/or CH3 domains). In some embodiments, the C-terminus of the Fe domain is another linker that is fused to the targeting moiety. Thus, in some embodiments, the molecule could be represented using the formula of R1-Linker A-Fc Region-Linker B-R2, wherein R1 can be an effector binding/modulating moiety, R2 is a targeting moiety, Linker A and Linker B are independently linkers as provided for herein. In some embodiments, Linker 1 and Linker 2 are different.

In some embodiments, the molecule could be represented using the formula of R1-Linker A-Fc Region-Linker B-R2, wherein R1 can be a targeting moiety, R2 is an effector binding/modulating moiety, Linker A and Linker B are independently linkers as provided for herein. In some embodiments, Linker A and Linker B are different. The linkers can be chosen from the non-limiting examples provided for herein. In some embodiments, R1 and R2 are independently selected from F(ab′)2 and scFV antibody domains. In some embodiments, R1 and R2 are different antibody domains. In some embodiments, the scFV is in the VL-VH domain orientation.

In some embodiments, the therapeutic compound is a bispecific antibody. In some embodiments, the bispecific antibodies are comprised of four polypeptide chains comprising the following:
Chain 1: nt-VH1-CH1-CH2-CH3-Linker A-scFv[VL2-Linker B-VH2]-ct
Chain 2: nt-VH1-CH1-CH2-CH3-Linker A-scFv[VL2-Linker B-VH2]-ct
Chain 3: nt-VL1-CL-ct
Chain 4: nt-VL1-CL-ct,
wherein chains 1 and 2 are identical to each other, and chains 3 and 4 are identical to each other,
wherein chain 1 forms a homodimer with chain 2; and chain 3 and 4 associate with chain 1 and chain 2. That is, when each light chain associates with each heavy chain, VL1 associates with VH1 and CL associates with CH1 to form two functional Fab units. Without being bound to any particular theory, each scFv unit is intrinsically functional since VL2 and VH2 are covalently linked in tandem with a linker as provided herein (e.g. GGGGS (SEQ ID NO: 23), GGGGSGGGGSGGGGSGGGGS (SEQ ID NO: 22), or GGGGSGGGGSGGGGS (SEQ ID NO: 30). The sequences of Linker A and Linker B, which are independent of one another can be the same or different and as otherwise described throughout the present application. Thus, in some embodiments, Linker A comprises GGGGS (SEQ ID NO: 23), or two repeats thereof, GGGGSGGGGSGGGGS (SEQ ID NO: 30), or GGGGSGGGGSGGGGSGGGGS (SEQ ID NO: 22). In some embodiments, Linker B comprises GGGGS (SEQ ID NO: 23), or two repeats thereof, GGGGSGGGGSGGGGS (SEQ ID NO: 30), or GGGGSGGGGSGGGGSGGGGS (SEQ ID NO: 22). The scFv may be arranged in the NT-VH2-VL2-CT or NT-VL2-VH2-CT orientation. NT or nt stands for N-terminus and CT or ct stands for C-terminus of the protein. CH1, CH2, and CH3 are the domains from the IgG Fc region, and CL stands for Constant Light chain, which can be either kappa or lambda family light chains. The other definitions stand for the way they are normally used in the art.

In some embodiments, the VH1 and VL1 domains are derived from the effector molecule and the VH2 and VL2 domains are derived from the targeting moiety. In some embodiments the VH1 and VL1 domains are derived from a targeting moiety and the VH2 and VL2 domains are derived from an effector binding/modulating moiety.

In some embodiments, the VH1 and VL1 domains are derived from an anti-PD-1 antibody, and the VH2 and VL2 domains are derived from an anti-MAdCAM antibody. In some embodiments the VH1 and VL1 domains are derived from an anti-MAdCAM antibody and the VH2 and VL2 domains are derived from an anti-PD-1 antibody.

In some embodiments, Linker A comprises 1, 2, 3, 4, or 5 GGGGS (SEQ ID NO: 23) repeats (repeats disclosed as SEQ ID NO: 1549). In some embodiments, Linker B comprises 1, 2, 3, 4, or 5 GGGGS (SEQ ID NO: 23) repeats (repeats disclosed as SEQ ID NO: 1549). For the avoidance of doubt, the sequences of Linker A and Linker B, which are used throughout this application, are independent of one another. Therefore, in some embodiments, Linker A and Linker B can be the same or different. In some embodiments, Linker A comprises GGGGS (SEQ ID NO: 23), or two repeats thereof, GGGGSGGGGSGGGGS (SEQ ID NO: 30), or GGGGSGGGGSGGGGSGGGGS (SEQ ID NO: 22). In some embodiments, Linker B comprises GGGGS (SEQ ID NO: 23), or two repeats thereof, GGGGSGGGGSGGGGS (SEQ ID NO: 30), or GGGGSGGGGSGGGGSGGGGS (SEQ ID NO: 22).

In some embodiments, the therapeutic compound comprises a light chain and a heavy chain. In some embodiments, the light and heavy chain begin at the N-terminus with the VH domain of a targeting moiety followed by the CH1 domain of a human IgG1, which is fused to a Fc region (e.g. CH2-CH3) of human IgG1. In some embodiments, at the c-terminus of the Fc region is fused to a linker as provided herein, such as but not limited to, GGGGS (SEQ ID NO: 23), or two or three repeats thereof, or GGGGSGGGGSGGGGS (SEQ ID NO: 30). The linker can then be fused to an effector binding/modulating moiety, such as any one of the effector moieties provided for herein. The polypeptides can homodimerize because through the heavy chain homodimerization, which results in a therapeutic compound having two effector moieties, such as two anti-PD-1 antibodies. In this orientation, the targeting moiety is an IgG format, there are two Fab arms that each recognize binding partner of the targeting moiety, for example, MAdCAM being bound by the anti-MAdCAM targeting moiety.

In some embodiments, the therapeutic or polypeptide comprises a formula of: An antibody (targeting moiety) with a variable heavy chain and a variable light chain, in an IgG isotype, for example, with an effector molecule, such as an IL-2 mutein. In some embodiments, the IL-2 mutein is fused at the c-terminus of the variable heavy chain. This can be represented by the formula of VL and VH-IgGConstantDomain-L1-E, wherein L1 is a linker, such as a glycine/serine linker as provided herein, E is an effector molecule, such as an IL-2 mutein and VL and VH are the variable light and heavy chains. The VL domain can be a kappa domain. In some embodiments, the IgG Constant domain comprises the sequence of:

(SEQ ID NO: 44)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGAPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPG.

In some embodiments, the linker comprises GGGGS (SEQ ID NO: 23). In some embodiments, the IL-2 mutein comprises the IL-2 muteins provided herein, such as one of SEQ ID NOs: 31-41, which can also have a Fc molecule appended to the N- or C-terminus of the IL-2 mutein. The Fc domain can comprise SEQ ID NO: 21 or 43. In some embodiments, the IL-2 mutein comprises one of SEQ ID NO: 47-60. In some embodiments, the IL-2 mutein comprises SEQ ID NO: 41 or SEQ ID NO: 56. In some embodiments, the IL-2 mutein comprises SEQ ID NO: 40 or SEQ ID NO: 55.

In some embodiments, the targeting moiety is a MAdCAM antibody.

In some embodiments, the MAdCAM antibody is selected from the following table

TABLE 6

| Clone | | | | | | | |
|---|---|---|---|---|---|---|---|
| ID | HCDR1 | HCDR2 | HCDR3 | LCDR1 | LCDR2 | LCDR3 | SCFv |
| 1. | FTFS | AVIS | CTTS | QASQDI | AASSLQS | CQQGYSTPLTF | EVQLLESGGGLVQPGGSLRLSCAA |
| | SYGM | DDGS | KYYY | SKSLN | (SEQ ID | (SEQ ID NO: | SGFTFSSYGMHWVRQAPGKGLEWV |
| | H | DKYY | YYGM | SEQ | NO: 65) | 66) | AVISDDGSDKYYADSVKGRFTISR |
| | (SEQ | A | DVW | ID NO: | | | DNSKNTLYLQMNSLRAEDTAVYYC |
| | ID | (SEQ | (SEQ | 64) | | | TTSKYYYYYGMDVWGQGTTVTVSS |
| | NO: | ID | ID | | | | GGGGSGGGGSGGGGSGGGGSDIQM |
| | 61) | NO: | NO: | | | | TQSPSSLSASVGDRVTITCQASQD |
| | | 62) | 63) | | | | ISKSLNWYQQKPGKAPKLLIYAAS |
| | | | | | | | SLQSGVPSRFSGSGSGTDFTLTIS |
| | | | | | | | SLQPEDFATYYCQQGYSTPLTFGG |
| | | | | | | | GTKVEIK (SEQ ID NO: 67) |
| 2. | YPFI | GIIN | CARE | RASQSI | GASTLES | CQQTWGPPFTF | QVQLVQSGAEVKKPGASVKVSCKA |
| | GYYL | PSGG | GRLS | SSYLA | (SEQ ID | (SEQ ID NO: | SGYPFIGYYLHWVRQAPGQGLEWM |
| | H | STSY | YGMD | (SEQ | NO: 72) | 73) | GIINPSGGSTSYAQKFQGRVTMTR |
| | (SEQ | A | AW | ID NO: | | | DTSTSTVYMELSSLRSEDTAVYYC |
| | ID | (SEQ | (SEQ | 71) | | | AREGRLSYGMDAWGQGTLVTVSSG |
| | NO: | ID | ID | | | | GGGSGGGGSGGGGSGGGGSDIQMT |
| | 68) | NO: | NO: | | | | QSPSSLSASVGDRVTITCRASQSI |
| | | 69) | 70) | | | | SSYLAWYQQKPGKAPKLLIYGAST |
| | | | | | | | LESGVPSRFSGSGSGTDFTLTISS |
| | | | | | | | LQPEDFATYYCQQTWGPPFTFGQG |
| | | | | | | | TKLEIK (SEQ ID NO: 74) |
| 3. | YPFI | GIIN | CARE | RASQSI | GASTLES | CQQTWGPPFTF | QVQLVQSGAEVKKPGASVKVSCKA |
| | GQYL | PSGG | GRLS | SSYLA | (SEQ ID | (SEQ ID NO: | SGYPFIGQYLHWVRQAPGQGLEWM |
| | H | STSY | YGMD | (SEQ | NO: 72) | 73) | GIINPSGGSTSYAQKFQGRVTMTR |
| | (SEQ | A | AW | ID NO: | | | DTSTSTVYMELSSLRSEDTAVYYC |
| | ID | (SEQ | (SEQ | 71) | | | AREGRLSYGMDAWGQGTLVTVSSG |
| | NO: | ID | ID | | | | GGGSGGGGSGGGGSGGGGSDIQMT |
| | 75) | NO: | NO: | | | | QSPSSLSASVGDRVTITCRASQSI |
| | | 69) | 70) | | | | SSYLAWYQQKPGKAPKLLIYGAST |
| | | | | | | | LESGVPSRFSGSGSGTDFTLTISS |
| | | | | | | | LQPEDFATYYCQQTWGPPFTFGQG |
| | | | | | | | TKLEIK (SEQ ID NO: 76) |
| 4. | GTFS | GSIN | CAKD | QASQDI | AASSLQS | CQQSYSSVITF | QVQLVQSGAEVKKPGASVKVSCKA |
| | SYAI | PSGD | KAQW | SNSLN | (SEQ ID | (SEQ ID NO: | SGGTFSSYAISWVRQAPGQGLEWM |
| | S | TTSY | LVGY | (SEQ | NO: 65) | 81) | GSINPSGDTTSYAQKFQGRVTMTR |
| | (SEQ | A | FDYW | ID NO: | | | DTSTSTVYMELSSLRSEDTAVYYC |
| | ID | (SEQ | (SEQ | 80) | | | AKDKAQWLVGYFDYWGQGTLVTVS |
| | NO: | ID | ID | | | | SGGGGSGGGGSGGGGSGGGGSDIQ |
| | 77) | NO: | NO: | | | | MTQSPSSLSASVGDRVTITCQASQ |
| | | 78) | 79) | | | | DISNSLNWYQQKPGKAPKLLIYAA |
| | | | | | | | SSLQSGVPSRFSGSGSGTDFTLTI |
| | | | | | | | SSLQPEDFATYYCQQSYSSVITFG |
| | | | | | | | QGTKVEIK (SEQ ID NO: 82) |
| 5. | FTFS | SSIS | CARE | RASQGI | GASSLQS | CQQANSFPFTF | EVQLLESGGGLVQPGGSLRLSCAA |
| | SYWM | PGGS | VQLS | SNSLA | (SEQ ID | (SEQ ID NO: | SGFTFSSYWMHWVRQAPGKGLEWV |
| | H | NIDY | HYDY | (SEQ | NO: 87) | 88) | SSISPGGSNIDYADSVKGRFTISR |
| | (SEQ | A | W | ID NO: | | | DNSKNTLYLQMNSLRAEDTAVYYC |
| | ID | (SEQ | (SEQ | 86) | | | AREVQLSHYDYWGQGTLVTVSSGG |
| | NO: | ID | ID | | | | GGSGGGGSGGGGSGGGGSDIQMTQ |
| | 83) | NO: | NO: | | | | SPSSLSASVGDRVTITCRASQGIS |
| | | 84) | 85) | | | | NSLAWYQQKPGKAPKLLIYGASSL |
| | | | | | | | QSGVPSRFSGSGSGTDFTLTISSL |
| | | | | | | | QPEDFATYYCQQANSFPFTFGQGT |
| | | | | | | | KVEIK (SEQ ID NO: 89) |
| 6. | FTFN | SRIN | CARE | RASQII | GASSLQS | CQQSYRLPFTF | EVQLLESGGGLVQPGGSLRLSCAA |
| | NYAF | SYGT | GPVA | GTNLA | (SEQ ID | (SEQ ID NO: | SGFTENNYAFHWVRQAPGKGLEWV |
| | H | STTY | GYWY | (SEQ | NO: 87) | 94) | SRINSYGTSTTYADSVKGRFTISR |
| | (SEQ | A | FDLW | ID NO: | | | DNSKNTLYLQMNSLRAEDTAVYYC |
| | ID | (SEQ | (SEQ | 93) | | | AREGPVAGYWYFDLWGQGTLVTVS |
| | NO: | ID | ID | | | | SGGGGSGGGGSGGGGSGGGGSDIQ |
| | 90) | NO: | NO: | | | | MTQSPSSLSASVGDRVTITCRASQ |
| | | 91) | 92) | | | | IIGTNLAWYQQKPGKAPKLLIYGA |
| | | | | | | | SSLQSGVPSRFSGSGSGTDFTLTI |
| | | | | | | | SSLQPEDFATYYCQQSYRLPFTFG |
| | | | | | | | QGTKVEIK (SEQ ID NO: 95) |
| 7. | YTFT | GIIN | CAKD | RASQNI | AASSLQS | CQQSYTTPYTF | QVQLVQSGAEVKKPGASVKVSCKA |
| | GYHI | PSGG | WSSW | SSSLN | (SEQ ID | (SEQ ID NO: | SGYTFTGYHIHWVRQAPGQGLEWM |
| | H | STIY | YLGP | (SEQ | NO: 65) | 100) | GIINPSGGSTIYAQKFQGRVTMTR |
| | (SEQ | A | FDYW | ID NO: | | | DTSTSTVYMELSSLRSEDTAVYYC |
| | ID | (SEQ | (SEQ | 99) | | | AKDWSSWYLGPFDYWGQGTLVTVS |
| | NO: | ID | ID | | | | SGGGGSGGGGSGGGGSGGGGSDIQ |
| | 96) | NO: | NO: | | | | MTQSPSSLSASVGDRVTITCRASQ |
| | | 97) | 98) | | | | NISSSLNWYQQKPGKAPKLLIYAA |
| | | | | | | | SSLQSGVPSRFSGSGSGTDFTLTI |
| | | | | | | | SSLQPEDFATYYCQQSYTTPYTFG |
| | | | | | | | QGTKVEIK (SEQ ID NO: |
| | | | | | | | 101) |
| 8. | FMFG | SAIS | CAKD | RASQGI | DASSLES | CQQTHSFPSTF | EVQLLESGGGLVQPGGSLRLSCAA |
| | DYAM | GSGG | LVVA | SNNLN | (SEQ ID | (SEQ ID NO: | SGFMFGDYAMHWVRQAPGKGLEWV |
| | H | STYY | GIWY | (SEQ | NO: | 107) | SAISGSGGSTYYADSVKGRFTISR |
| | (SEQ | A | FDLW | ID NO: | 106) | | DNSKNTLYLQMNSLRAEDTAVYYC |
| | ID | (SEQ | (SEQ | 105) | | | AKDLVVAGIWYFDLWGRGTLVTVS |
| | NO: | ID | ID | | | | SGGGGSGGGGSGGGGSGGGGSDIQ |
| | 102) | NO: | NO: | | | | MTQSPSSLSASVGDRVTITCRASQ |
| | | 103) | 104) | | | | GISNNLNWYQQKPGKAPKLLIYDA |
| | | | | | | | SSLESGVPSRFSGSGSGTDFTLTI |
| | | | | | | | SSLQPEDFATYYCQQTHSFPSTFG |
| | | | | | | | QGTKLEIK (SEQ ID NO: |
| | | | | | | | 108) |
| 9. | FTFS | SVIG | CAAD | RASQGI | AASTLQS | CQQSYSTPWTF | EVQLLESGGGLVQPGGSLRLSCAA |
| | DYYM | ESGG | PVSR | SSSLA | (SEQ ID | (SEQ ID NO: | SGFTFSDYYMNWVRQAPGKGLEWV |
| | N | STYY | WPKH | (SEQ | NO: | 114) | SVIGESGGSTYYADSVKGRFTISR |
| | (SEQ | A | GGGD | ID NO: | 113) | | DNSKNTLYLQMNSLRAEDTAVYYC |
| | ID | (SEQ | YW | 112) | | | AADPVSRWPKHGGGDYWGQGTLVT |
| | NO: | ID | (SEQ | | | | VSSGGGGSGGGGSGGGGSGGGGSD |
| | 109) | NO: | ID | | | | IQMTQSPSSLSASVGDRVTITCRA |
| | | 110) | NO: | | | | SQGISSSLAWYQQKPGKAPKLLIY |
| | | | 111) | | | | AASTLQSGVPSRFSGSGSGTDFTL |
| | | | | | | | TISSLQPEDFATYYCQQSYSTPWT |
| | | | | | | | FGQGTKVEIK (SEQ ID NO: |
| | | | | | | | 115) |
| 10. | YTLT | GWIN | CAKG | RASDNI | AASSLQS | CQQGYSTPPTF | QVQLVQSGAEVKKPGASVKVSCKA |
| | TWYM | PNRG | DLWG | GSWLA | (SEQ ID | (SEQ ID NO: | SGYTLTTWYMYWVRQAPGQGLEWM |
| | Y | ATNY | AMDV | (SEQ | NO: 65) | 120) | GWINPNRGATNYAQKFQGRVTMTR |
| | (SEQ | A | W | ID NO: | | | DTSTSTVYMELSSLRSEDTAVYYC |
| | ID | (SEQ | (SEQ | 119) | | | AKGDLWGAMDVWGQGTLVTVSSGG |
| | NO: | ID | ID | | | | GGSGGGGSGGGGSGGGGSDIQMTQ |
| | 116) | NO: | NO: | | | | SPSSLSASVGDRVTITCRASDNIG |
| | | 117) | 118) | | | | SWLAWYQQKPGKAPKLLIYAASSL |
| | | | | | | | QSGVPSRFSGSGSGTDFTLTISSL |
| | | | | | | | QPEDFATYYCQQGYSTPPTFGQGT |
| | | | | | | | KVEIK (SEQ ID NO: 121) |
| 11. | YTFT | GGFD | CARH | RASESI | AASTLQS | CQQSYSVPFTF | QVQLVQSGAEVKKPGASVKVSCKA |
| | TYYM | PEDG | AVAG | SNWLA | (SEQ ID | (SEQ ID NO: | SGYTFTTYYMHWVRQAPGQGLEWM |
| | H | ETIY | AVGA | (SEQ | NO: | 126) | GGFDPEDGETIYAQKFQGRVTMTR |
| | (SEQ | A | GYYY | ID NO: | 113) | | DTSTSTVYMELSSLRSEDTAVYYC |
| | ID | (SEQ | YGMD | 125) | | | ARHAVAGAVGAGYYYYGMDVWGQG |
| | NO: | ID | VW | | | | TMVTVSSGGGGSGGGGSGGGGSGG |
| | 122) | NO: | (SEQ | | | | GGSDIQMTQSPSSLSASVGDRVTI |
| | | 123) | ID | | | | TCRASESISNWLAWYQQKPGKAPK |
| | | | NO: | | | | LLIYAASTLQSGVPSRFSGSGSGT |
| | | | 124) | | | | DFTLTISSLQPEDFATYYCQQSYS |
| | | | | | | | VPFTFGPGTKVDIK (SEQ ID |
| | | | | | | | NO: 127) |
| 12. | YTFT | GWIG | CARD | RSSQSL | SSSNRAP | CMQALHIPLTF | QVQLVQSGAEVKKPGASVKVSCKA |
| | GYYM | PNSG | LDHN | LHSNGY | (SEQ ID | (SEQ ID NO: | SGYTFTGYYMHWVRQAPGQGLEWM |
| | H | DTNY | WYFD | NYLD | NO: | 133) | GWIGPNSGDTNYAQKFQGRVTMTR |
| | (SEQ | A | LW | (SEQ | 132) | | DTSTSTVYMELSSLRSEDTAVYYC |
| | ID | (SEQ | (SEQ | ID NO: | | | ARDLDHNWYFDLWGRGTLVTVSSG |
| | NO: | ID | ID | 131) | | | GGGSGGGGSGGGGSGGGGSDIVMT |
| | 128) | NO: | NO: | | | | QSPLSLPVTPGEPASISCRSSQSL |
| | | 129 | 130) | | | | LHSNGYNYLDWYLQKPGQSPQLLI |
| | | | | | | | YSSSNRAPGVPDRFSGSGSGTDFT |
| | | | | | | | LKISRVEAEDVGVYYCMQALHIPL |
| | | | | | | | TFGGGTKVEIK (SEQ ID NO: |
| | | | | | | | 134) |
| 13. | FTFD | SYID | CAKD | QASQDI | KASTLES | CQQSYSTPITF | EVQLLESGGGLVQPGGSLRLSCAA |
| | DYAM | ASGT | QAAA | SNYLN | (SEQ ID | (SEQ ID NO: | SGFTFDDYAMHWVRQAPGKGLEWV |
| | H | TIYY | GYWY | (SEQ | NO: | 140) | SYIDASGTTIYYADSVKGRFTISR |
| | (SEQ | A | FDLW | ID NO: | 139 | | DNSKNTLYLQMNSLRAEDTAVYYC |
| | ID | (SEQ | (SEQ | 138) | | | AKDQAAAGYWYFDLWGRGTLVTVS |
| | NO: | ID | ID | | | | SGGGGSGGGGSGGGGSGGGGSDIQ |
| | 135) | NO: | NO: | | | | MTQSPSSLSASVGDRVTITCQASQ |
| | | 136) | 137) | | | | DISNYLNWYQQKPGKAPKLLIYKA |
| | | | | | | | STLESGVPSRFSGSGSGTDFTLTI |
| | | | | | | | SSLQPEDFATYYCQQSYSTPITFG |
| | | | | | | | QGTRLEIK (SEQ ID NO: |
| | | | | | | | 141) |
| 14. | YTFT | GGIV | CAKD | RSSQSL | SAYNRAS | CMQALQTPLTF | QVQLVQSGAEVKKPGSSVKVSCKA |
| | DYHI | PRSG | ESSG | LHSNGY | (SEQ ID | (SEQ ID NO: | SGYTFTDYHIHWVRQAPGQGLEWM |
| | H | STTY | WYYF | NYLD | NO: | 146) | GGIVPRSGSTTYAQKFQGRVTITA |
| | (SEQ | A | DYW | (SEQ | 145) | | DESTSTAYMELSSLRSEDTAVYYC |
| | ID | (SEQ | (SEQ | ID NO: | | | AKDESSGWYYFDYWGQGTLVTVSS |
| | NO: | ID | ID | 131) | | | GGGGSGGGGSGGGGSGGGGSDIVM |
| | 142) | NO: | NO: | | | | TQSPLSLPVTPGEPASISCRSSQS |
| | | 143) | 144) | | | | LLHSNGYNYLDWYLQKPGQSPQLL |
| | | | | | | | IYSAYNRASGVPDRFSGSGSGTDF |
| | | | | | | | TLKISRVEAEDVGVYYCMQALQTP |
| | | | | | | | LTFGQGTKVEIK (SEQ ID NO: |
| | | | | | | | 147) |
| 15. | YTFT | GGII | CAKG | QANQDI | RASKLEA | CQQSSEIPYSF | QVQLVQSGAEVKKPGSSVKVSCKA |
| | NYYM | PIVD | RYTV | SNYLN | (SEQ ID | (SEQ ID NO: | SGYTFTNYYMHWVRQAPGQGLEWM |
| | H | RVKY | NYYY | (SEQ | NO: | 153) | GGIIPIVDRVKYAQKFQGRVTITA |
| | (SEQ | A | GMDV | ID NO: | 152) | | DESTSTAYMELSSLRSEDTAVYYC |
| | ID | (SEQ | W | 151) | | | AKGRYTVNYYYGMDVWGQGTTVTV |
| | NO: | ID | (SEQ | | | | SSGGGGSGGGGSGGGGSGGGGSDI |
| | 148) | NO: | ID | | | | QMTQSPSSLSASVGDRVTITCQAN |
| | | 149) | NO: | | | | QDISNYLNWYQQKPGKAPKLLIYR |
| | | | 150) | | | | ASKLEAGVPSRFSGSGSGTDFTLT |
| | | | | | | | ISSLQPEDFATYYCQQSSEIPYSF |
| | | | | | | | GQGTKLEIK (SEQ ID NO: |
| | | | | | | | 154) |
| 16. | FTFE | SYLN | CAKD | RASQSI | DASNLET | CQQSYTIPITF | EVQLLESGGGLVQPGGSLRLSCAA |
| | DYAM | SDGG | YCTN | STYLN | (SEQ ID | (SEQ ID NO: | SGFTFEDYAMHWVRQAPGKGLEWV |
| | H | STSY | GVCA | (SEQ | NO: | 160) | SYLNSDGGSTSYADSVKGRFTISR |
| | (SEQ | A | FDYW | ID NO: | 159) | | DNSKNTLYLQMNSLRAEDTAVYYC |
| | ID | (SEQ | (SEQ | 158) | | | AKDYCTNGVCAFDYWGQGTLVTVS |
| | NO: | ID | ID | | | | SGGGGSGGGGSGGGGSGGGGSDIQ |
| | 155) | NO: | NO: | | | | MTQSPSSLSASVGDRVTITCRASQ |
| | | 156) | 157) | | | | SISTYLNWYQQKPGKAPKLLIYDA |
| | | | | | | | SNLETGVPSRFSGSGSGTDFTLTI |
| | | | | | | | SSLQPEDFATYYCQQSYTIPITFG |
| | | | | | | | QGTRLEIK (SEQ ID NO: |
| | | | | | | | 161) |
| 17. | FTFS | SAIS | CVSD | RASQSI | AASRLEG | CQQANSFPLTF | EVQLLESGGGLVQPGGSLRLSCAA |
| | DSAM | GSGS | IAVA | STFLN | (SEQ ID | (SEQ ID NO: | SGFTFSDSAMHWVRQAPGKGLEWV |
| | H | TIYY | GHWY | (SEQ | NO: | 167) | SAISGSGSTIYYADSVKGRFTISR |
| | (SEQ | A | FDLW | ID NO: | 166) | | DNSKNTLYLQMNSLRAEDTAVYYC |
| | ID | (SEQ | (SEQ | 165) | | | VSDIAVAGHWYFDLWGRGTLVTVS |
| | NO: | ID | ID | | | | SGGGGSGGGGSGGGGSGGGGSDIQ |
| | 162) | NO: | NO: | | | | MTQSPSSLSASVGDRVTITCRASQ |
| | | 163) | 164) | | | | SISTFLNWYQQKPGKAPKLLIYAA |
| | | | | | | | SRLEGGVPSRFSGSGSGTDFTLTI |
| | | | | | | | SSLQPEDFATYYCQQANSFPLTFG |
| | | | | | | | PGTKVDIK (SEQ ID NO: |
| | | | | | | | 168) |
| 18. | FTFS | SYIS | CARA | RASQSI | AASSLQS | CQQSYSTPLTF | EVQLVESGGGLVKPGGSLRLSCAA |
| | SYWM | GDSG | NSSG | SSYLN | (SEQ ID | (SEQ ID NO: | SGFTFSSYWMSWVRQAPGKGLEWV |
| | S | YTNY | WYDW | (SEQ | NO: 65) | 173) | SYISGDSGYTNYAAPVKGRFTISR |
| | (SEQ | A | YFDL | ID NO: | | | DDSKNTLYLQMNSLKTEDTAVYYC |
| | ID | (SEQ | W | 172) | | | ARANSSGWYDWYFDLWGRGTLVTV |
| | NO: | ID | (SEQ | | | | SSGGGGSGGGGSGGGGSGGGGSDI |
| | 169) | NO: | ID | | | | QMTQSPSSLSASVGDRVTITCRAS |
| | | 170) | NO: | | | | QSISSYLNWYQQKPGKAPKLLIYA |
| | | | 171) | | | | ASSLQSGVPSRFSGSGSGTDFTLT |
| | | | | | | | ISSLQPEDFATYYCQQSYSTPLTF |
| | | | | | | | GGGTKVEIK (SEQ ID NO: |
| | | | | | | | 174) |
| 19. | FTFD | SGIS | CAKD | QASQDI | DASNLET | CQQSYSTPLTF | EVQLLESGGGLVQPGGSLRLSCAA |
| | DYAM | WNSG | IVAA | SNYLN | (SEQ ID | (SEQ ID NO: | SGFTFDDYAMHWVRQAPGKGLEWV |
| | H | SIGY | GHYY | (SEQ | NO: | 173) | SGISWNSGSIGYADSVKGRFTISR |
| | (SEQ | A | YGMD | ID NO: | 159) | | DNSKNTLYLQMNSLRAEDTAVYYC |
| | ID | (SEQ | VW | 138) | | | AKDIVAAGHYYYGMDVWGQGTTVT |
| | NO: | ID | (SEQ | | | | VSSGGGGSGGGGSGGGGSGGGGSD |
| | 135) | NO: | ID | | | | IQMTQSPSSLSASVGDRVTITCQA |
| | | 175) | NO: | | | | SQDISNYLNWYQQKPGKAPKLLIY |
| | | | 176) | | | | DASNLETGVPSRFSGSGSGTDFTL |
| | | | | | | | TISSLQPEDFATYYCQQSYSTPLT |
| | | | | | | | FGGGTKVEIK (SEQ ID NO: |
| | | | | | | | 177) |
| 20. | FTFD | SYID | CARD | QAGQDI | DASNLET | CQQTYSTPITF | EVQLLESGGGLVQPGGSLRLSCAA |
| | DYAM | TSSS | EAAA | SNYLN | (SEQ ID | (SEQ ID NO: | SGFTFDDYAMHWVRQAPGKGLEWV |
| | H | HLYY | GYYG | (SEQ | NO: | 181) | SYIDTSSSHLYYADSVKGRFTISR |
| | (SEQ | A | MDVW | ID NO: | 159) | | DNSKNTLYLQMNSLRAEDTAVYYC |
| | ID | (SEQ | (SEQ | 180) | | | ARDEAAAGYYGMDVWGQGTTVTVS |
| | NO: | ID | ID | | | | SGGGGSGGGGSGGGGSGGGGSDIQ |
| | 135) | NO: | NO: | | | | MTQSPSSLSASVGDRVTITCQAGQ |
| | | 178) | 179) | | | | DISNYLNWYQQKPGKAPKLLIYDA |
| | | | | | | | SNLETGVPSRFSGSGSGTDFTLTI |
| | | | | | | | SSLQPEDFATYYCQQTYSTPITFG |
| | | | | | | | QGTKLEIK (SEQ ID NO: |
| | | | | | | | 182) |
| 21. | FTFS | STIV | CARD | RASQDI | AASSLQS | CQQSYSIPPTF | EVQLLESGGGLVQPGGSLRLSCAA |
| | NAWM | GNGG | NPLR | SNYLN | (SEQ ID | (SEQ ID NO: | SGFTFSNAWMSWVRQAPGKGLEWV |
| | S | ATYY | WQGM | (SEQ | NO: 65) | 187) | STIVGNGGATYYADSVKGRFTISR |
| | (SEQ | A | DVW | ID NO: | | | DNSKNTLYLQMNSLRAEDTAVYYC |
| | ID | (SEQ | (SEQ | 186) | | | ARDNPLRWQGMDVWGQGTLVTVSS |
| | NO: | ID | ID | | | | GGGGSGGGGSGGGGSGGGGSDIQM |
| | 183) | NO: | NO: | | | | TQSPSSLSASVGDRVTITCRASQD |
| | | 184) | 185) | | | | ISNYLNWYQQKPGKAPKLLIYAAS |
| | | | | | | | SLQSGVPSRFSGSGSGTDFTLTIS |
| | | | | | | | SLQPEDFATYYCQQSYSIPPTFGP |
| | | | | | | | GTKVDIK (SEQ ID NO: 188) |
| 22. | FTFS | SYIS | CARA | RASQSI | AASSLQS | CQQSYSTPLTF | EVQLLESGGGLVQPGGSLRLSCAA |
| | SYQM | SSST | NSSS | SSYLN | (SEQ ID | (SEQ ID NO: | SGFTFSSYQMSWVRQAPGKGLEWV |
| | S | YTNY | WYDW | SEQ | NO: 65) | 173) | SYISSSSTYTNYADSVKGRFTISR |
| | (SEQ | A | YFDL | ID NO: | | | DNSKNTLYLQMNSLRAEDTAVYYC |
| | ID | (SEQ | W | 172) | | | ARANSSSWYDWYFDLWGQGTLVTV |
| | NO: | ID | (SEQ | | | | SSGGGGSGGGGSGGGGSGGGGSDI |
| | 189) | NO: | ID | | | | QMTQSPSSLSASVGDRVTITCRAS |
| | | 190) | NO: | | | | QSISSYLNWYQQKPGKAPKLLIYA |
| | | | 191) | | | | ASSLQSGVPSRFSGSGSGTDFTLT |
| | | | | | | | ISSLQPEDFATYYCQQSYSTPLTF |
| | | | | | | | GGGTKVEIK (SEQ ID NO: |
| | | | | | | | 192) |
| 23. | FTFS | SGIS | CATS | RASQSI | AASNLQR | CQQSYSIPITF | EVQLLESGGGLVQPGGSLRLSCAA |
| | SYAM | GSGG | QAPV | SSWLA | (SEQ ID | (SEQ ID NO: | SGFTFSSYAMHWVRQAPGKGLEWV |
| | H | SAYY | DYYY | (SEQ | NO: | 198) | SGISGSGGSAYYADSVKGRFTISR |
| | (SEQ | A | YGMD | ID NO: | 197) | | DNSKNTLYLQMNSLRAEDTAVYYC |
| | ID | (SEQ | VW | 196) | | | ATSQAPVDYYYYGMDVWGQGTTVT |
| | NO: | ID | (SEQ | | | | VSSGGGGSGGGGSGGGGSGGGGSD |
| | 193) | NO: | ID | | | | IQMTQSPSSLSASVGDRVTITCRA |
| | | 194) | NO: | | | | SQSISSWLAWYQQKPGKAPKLLIY |
| | | | 195 | | | | AASNLQRGVPSRFSGSGSGTDFTL |
| | | | | | | | TISSLQPEDFATYYCQQSYSIPIT |
| | | | | | | | FGQGTKVEIK (SEQ ID NO: |
| | | | | | | | 199) |
| 24. | FTFS | SYIS | CARV | RASQSI | AASSLQS | CQQSYSTPLTF | EVQLVESGGGLVKPGGSLRLSCAA |
| | SYWM | GSSS | GSSG | SSYLN | (SEQ ID | (SEQ ID NO: | SGFTFSSYWMSWVRQAPGKGLEWV |
| | S | YTNY | WYDW | SEQ | NO: 65) | 173) | SYISGSSSYTNYAAPVKGRFTISR |
| | (SEQ | A | YFDL | ID NO: | | | DDSKNTLYLQMNSLKTEDTAVYYC |
| | ID | (SEQ | W | 172) | | | ARVGSSGWYDWYFDLWGRGTLVTV |
| | NO: | ID | (SEQ | | | | SSGGGGSGGGGSGGGGSGGGGSDI |
| | 169) | NO: | ID | | | | QMTQSPSSLSASVGDRVTITCRAS |
| | | 200) | NO: | | | | QSISSYLNWYQQKPGKAPKLLIYA |
| | | | 201) | | | | ASSLQSGVPSRFSGSGSGTDFTLT |
| | | | | | | | ISSLQPEDFATYYCQQSYSTPLTF |
| | | | | | | | GQGTKVEIK (SEQ ID NO: |
| | | | | | | | 202) |
| 25. | YTLT | GWIN | CAKG | RASDNI | AASSLQS | CQQGYSTPPTF | QVQLVQSGAEVKKPGASVKVSCKA |
| | TWYM | PNRG | DLWG | GSWLA | (SEQ ID | (SEQ ID NO: | SGYTLTTWYMYWVRQAPGQGLEWM |
| | Y | ATNY | AMDV | (SEQ | NO: 65) | 120) | GWINPNRGATNYAQKFQGRVTMTR |
| | (SEQ | A | W | ID NO: | | | DTSTSTVYMELSSLRSEDTAVYYC |
| | ID | (SEQ | (SEQ | 119) | | | AKGDLWGAMDVWGQGTLVTVSSGG |
| | NO: | ID | ID | | | | GGSGGGGSGGGGSGGGGSDIQMTQ |
| | 116) | NO: | NO: | | | | SPSSLSASVGDRVTITCRASDNIG |
| | | 117) | 118) | | | | SWLAWYQQKPGKAPKLLIYAASSL |
| | | | | | | | QSGVPSRFSGSGSGTDFTLTISSL |
| | | | | | | | QPEDFATYYCQQGYSTPPTFGQGT |
| | | | | | | | KVEIK (SEQ ID NO: 121) |
| 26. | YTLT | GWIN | CAKG | RASDNI | AASSLQS | CQQGYSTPPTF | QVQLVQSGAEVKKPGASVKVSCKA |
| | TWYM | PNRG | DLWG | GSWLA | (SEQ ID | (SEQ ID NO: | SGYTLTTWYMYWVRQAPGQGLEWM |
| | Y | ATNY | AMDV | (SEQ | NO: 65) | 120) | GWINPNRGATNYAQKFQGRVTMTR |
| | (SEQ | A | W | ID NO: | | | DTSTSTVYMELSSLRSEDTAVYYC |
| | ID | (SEQ | (SEQ | 119) | | | AKGDLWGAMDVWGQGTTVTVSSGG |
| | NO: | ID | ID | | | | GGSGGGGSGGGGSGGGGSDIQMTQ |
| | 116) | NO: | NO: | | | | SPSSLSASVGDRVTITCRASDNIG |
| | | 117) | 118) | | | | SWLAWYQQKPGKAPKLLIYAASSL |
| | | | | | | | QSGVPSRFSGSGSGTDFTLTISSL |
| | | | | | | | QPEDFATYYCQQGYSTPPTFGQGT |
| | | | | | | | KVEIK (SEQ ID NO: 203) |
| 27. | YTFT | GWMN | CARD | RASQSI | AASSLQS | CQQSYTAPYTF | QVQLVQSGAEVKKPGASVKVSCKA |
| | GYYI | PNSG | PGFL | SSYLH | (SEQ ID | (SEQ ID NO: | SGYTFTGYYIHWVRQAPGQGLEWM |
| | H | NTGY | GYCS | (SEQ | NO: 65) | 208) | GWMNPNSGNTGYAQKFQGRVTMTR |
| | (SEQ | A | GGSC | ID NO: | | | DTSTSTVYMELSSLRSEDTAVYYC |
| | ID | (SEQ | YDGW | 207) | | | ARDPGFLGYCSGGSCYDGWFDPWG |
| | NO: | ID | FDPW | | | | QGTLVTVSSGGGGSGGGGSGGGGS |
| | 204) | NO: | (SEQ | | | | GGGGSDIQMTQSPSSLSASVGDRV |
| | | 205) | ID | | | | TITCRASQSISSYLHWYQQKPGKA |
| | | | NO: | | | | PKLLIYAASSLQSGVPSRFSGSGS |
| | | | 206) | | | | GTDFTLTISSLQPEDFATYYCQQS |
| | | | | | | | YTAPYTFGQGTKLEIK (SEQ ID |
| | | | | | | | NO: 209) |
| 28. | YTFT | GWMN | CARE | RASQGI | DASNLET | CQQSYSTPLTF | QVQLVQSGAEVKKPGASVKVSCKA |
| | DYFL | PTSG | GEGS | NSWLA | (SEQ ID | (SEQ ID NO: | SGYTFTDYFLHWVRQAPGQGLEWM |
| | H | NTGY | GFDY | (SEQ | NO: | 173) | GWMNPTSGNTGYAQKFQGRVTMTR |
| | (SEQ | A | W | ID NO: | 159) | | DTSTSTVYMELSSLRSEDTAVYYC |
| | ID | (SEQ | (SEQ | 213) | | | AREGEGSGFDYWGQGTLVTVSSGG |
| | NO: | ID | ID | | | | GGSGGGGSGGGGSGGGGSDIQMTQ |
| | 210) | NO: | NO: | | | | SPSSLSASVGDRVTITCRASQGIN |
| | | 211) | 212) | | | | SWLAWYQQKPGKAPKLLIYDASNL |
| | | | | | | | ETGVPSRFSGSGSGTDFTLTISSL |
| | | | | | | | QPEDFATYYCQQSYSTPLTFGGGT |
| | | | | | | | KVEIK (SEQ ID NO: 214) |
| 29. | YTFT | AWMN | CARD | RASQGI | AASSLQS | CQQSYSTPWTF | QVQLVQSGAEVKKPGASVKVSCKA |
| | SYYM | PNSG | YDFW | SNYLA | (SEQ ID | (SEQ ID NO: | SGYTFTSYYMHWVRQAPGQGLEWM |
| | H | NTGY | SGSL | (SEQ | NO: 65) | 114) | AWMNPNSGNTGYAQKFQGRVTMTR |
| | (SEQ | A | GYW | ID NO: | | | DTSTSTVYMELSSLRSEDTAVYYC |
| | ID | (SEQ | (SEQ | 218) | | | ARDYDFWSGSLGYWGQGTLVTVSS |
| | NO: | ID | ID | | | | GGGGSGGGGSGGGGSGGGGSDIQM |
| | 215 | NO: | NO: | | | | TQSPSSLSASVGDRVTITCRASQG |
| | | 216) | 217) | | | | ISNYLAWYQQKPGKAPKLLIYAAS |
| | | | | | | | SLQSGVPSRFSGSGSGTDFTLTIS |
| | | | | | | | SLQPEDFATYYCQQSYSTPWTFGQ |
| | | | | | | | GTKVEIK (SEQ ID NO: 219) |
| 30. | YTLT | GWIN | CAKG | RASDNI | AASSLQS | CQQGYSTPPTF | QVQLVQSGAEVKKPGASVKVSCKA |
| | TWYM | PNRG | DLWG | GSWLA | (SEQ ID | (SEQ ID NO: | SGYTLTTWYMYWVRQAPGQGLEWM |
| | Y | ATNY | AMDV | (SEQ | NO: 65) | 120) | GWINPNRGATNYAQKFQGRVTMTR |
| | (SEQ | A | W | ID NO: | | | DTSTSTVYMELSSLRSEDTAVYYC |
| | ID | (SEQ | (SEQ | 119) | | | AKGDLWGAMDVWGQGTLVTVSSGG |
| | NO: | ID | ID | | | | GGSGGGGSGGGGSGGGGSDIQMTQ |
| | 116) | NO: | NO: | | | | SPSSLSASVGDRVTITCRASDNIG |
| | | 117) | 118) | | | | SWLAWYQQKPGKAPKLLIYAASSL |
| | | | | | | | QSGVPSRFSGSGSGTDFTLTISSL |
| | | | | | | | QPEDFATYYCQQGYSTPPTFGQGT |
| | | | | | | | KVEIK (SEQ ID NO: 121) |
| 31. | YTFT | GIIN | CARD | RASQSI | DASNLQS | CQQSYSIPITF | QVQLVQSGAEVKKPGASVKVSCKA |
| | SYYM | PSGG | TGYS | GRWLA | (SEQ ID | (SEQ ID NO: | SGYTFTSYYMHWVRQAPGQGLEWM |
| | H | STSY | YGRY | (SEQ | NO: | 198) | GIINPSGGSTSYAQKFQGRVTMTR |
| | (SEQ | A | YYYG | ID NO: | 222) | | DTSTSTVYMELSSLRSEDTAVYYC |
| | ID | (SEQ | MDVW | 221) | | | ARDTGYSYGRYYYYGMDVWGQGTL |
| | NO: | ID | (SEQ | | | | VTVSSGGGGSGGGGSGGGGSGGGG |
| | 215) | NO: | ID | | | | SDIQMTQSPSSLSASVGDRVTITC |
| | | 69) | NO: | | | | RASQSIGRWLAWYQQKPGKAPKLL |
| | | | 220) | | | | IYDASNLQSGVPSRFSGSGSGTDF |
| | | | | | | | TLTISSLQPEDFATYYCQQSYSIP |
| | | | | | | | ITFGQGTKVEIK (SEQ ID NO: |
| | | | | | | | 223) |
| 32. | YTLT | GIIN | CARE | RASQGI | AASSLQS | CQQSYSTPLTF | QVQLVQSGAEVKKPGASVKVSCKA |
| | DYYM | PSGG | EYSS | SSWLA | (SEQ ID | (SEQ ID NO: | SGYTLTDYYMHWVRQAPGQGLEWM |
| | H | STSY | SSGY | SEQ | NO: 65) | 173) | GIINPSGGSTSYAQKFQGRVTMTR |
| | (SEQ | A | FDYW | ID NO: | | | DTSTSTVYMELSSLRSEDTAVYYC |
| | ID | (SEQ | (SEQ | 226) | | | AREEYSSSSGYFDYWGQGTLVTVS |
| | NO: | ID | ID | | | | SGGGGSGGGGSGGGGSGGGGSDIQ |
| | 224) | NO: | NO: | | | | MTQSPSSLSASVGDRVTITCRASQ |
| | | 69) | 225) | | | | GISSWLAWYQQKPGKAPKLLIYAA |
| | | | | | | | SSLQSGVPSRFSGSGSGTDFTLTI |
| | | | | | | | SSLQPEDFATYYCQQSYSTPLTFG |
| | | | | | | | QGTKVEIK (SEQ ID NO: |
| | | | | | | | 227) |
| 33. | YTFT | GWMH | CARD | RASQSI | AASSLQS | CQQSYSVPITF | QVQLVQSGAEVKKPGASVKVSCKA |
| | SYGI | PKSG | TPYY | SSWLA | (SEQ ID | (SEQ ID NO: | SGYTFTSYGISWVRQAPGQGLEWM |
| | S | DTGL | YYGM | (SEQ | NO: 65) | 231) | GWMHPKSGDTGLTQKFQGRVTMTR |
| | (SEQ | T | DVW | ID NO: | | | DTSTSTVYMELSSLRSEDTAVYYC |
| | ID | (SEQ | (SEQ | 196) | | | ARDTPYYYYGMDVWGQGTTVTVSS |
| | NO: | ID | ID | | | | GGGGSGGGGSGGGGSGGGGSDIQM |
| | 228) | NO: | NO: | | | | TQSPSSLSASVGDRVTITCRASQS |
| | | 229) | 230) | | | | ISSWLAWYQQKPGKAPKLLIYAAS |
| | | | | | | | SLQSGVPSRFSGSGSGTDFTLTIS |
| | | | | | | | SLQPEDFATYYCQQSYSVPITFGQ |
| | | | | | | | GTKVEIK (SEQ ID NO: 232) |
| 34. | FTFG | SYIS | CARD | RASQSI | AASSLQS | CQQSYSTPLTF | EVQLVESGGGLVKPGGSLRLSCAA |
| | DYAM | GDIG | VAAT | SSYLN | (SEQ ID | (SEQ ID NO: | SGFTFGDYAMSWVRQAPGKGLEWV |
| | S | YTNY | GNWY | (SEQ | NO: 65) | 173) | SYISGDIGYTNYAAPVKGRFTISR |
| | (SEQ | A | FDLW | ID NO: | | | DDSKNTLYLQMNSLKTEDTAVYYC |
| | ID | (SEQ | (SEQ | 172) | | | ARDVAATGNWYFDLWGRGTLVTVS |
| | NO: | ID | ID | | | | SGGGGSGGGGSGGGGSGGGGSDIQ |
| | 233) | NO: | NO: | | | | MTQSPSSLSASVGDRVTITCRASQ |
| | | 234) | 235) | | | | SISSYLNWYQQKPGKAPKLLIYAA |
| | | | | | | | SSLQSGVPSRFSGSGSGTDFTLTI |
| | | | | | | | SSLQPEDFATYYCQQSYSTPLTFG |
| | | | | | | | GGTKVEIK (SEQ ID NO: |
| | | | | | | | 236) |
| 35. | FSFS | SFIT | CARD | RASQSV | GASTRAT | CQQYGSSPLTF | EVQLLESGGGLVQPGGSLRLSCAA |
| | SYTM | SSSR | RRGD | RNYLA | (SEQ ID | (SEQ ID NO: | SGFSFSSYTMNWVRQAPGKGLEWV |
| | N | TIYY | YGDS | (SEQ | NO: | 242) | SFITSSSRTIYYADSVKGRFTISR |
| | (SEQ | A | WYFD | ID NO: | 241) | | DNSKNTLYLQMNSLRAEDTAVYYC |
| | ID | (SEQ | LW | 240) | | | ARDRRGDYGDSWYFDLWGRGTLVT |
| | NO: | ID | (SEQ | | | | VSSGGGGSGGGGSGGGGSGGGGSE |
| | 237) | NO: | ID | | | | IVMTQSPATLSVSPGERATLSCRA |
| | | 238) | NO: | | | | SQSVRNYLAWYQQKPGQAPRLLIY |
| | | | 239) | | | | GASTRATGIPARFSGSGSGTEFTL |
| | | | | | | | TISSLQSEDFAVYYCQQYGSSPLT |
| | | | | | | | FGGGTKVEIK (SEQ ID NO: |
| | | | | | | | 243) |
| 36. | YTFT | GIIN | CARD | RASQSI | DASNLQS | CQQSYSIPITF | QVQLVQSGAEVKKPGASVKVSCKA |
| | GHYM | PSGG | TGYS | GRWLA | (SEQ ID | (SEQ ID NO: | SGYTFTGHYMHWVRQAPGQGLEWM |
| | H | STSY | YGRY | (SEQ | NO: | 198) | GIINPSGGSTSYAQKFQGRVTMTR |
| | (SEQ | A | YYYG | ID NO: | 222) | | DTSTSTVYMELSSLRSEDTAVYYC |
| | ID | (SEQ | MDVW | 221) | | | ARDTGYSYGRYYYYGMDVWGQGTT |
| | NO: | ID | (SEQ | | | | VTVSSGGGGSGGGGSGGGGSGGGG |
| | 244) | NO: | ID | | | | SDIQMTQSPSSLSASVGDRVTITC |
| | | 69) | NO: | | | | RASQSIGRWLAWYQQKPGKAPKLL |
| | | | 220 | | | | IYDASNLQSGVPSRFSGSGSGTDE |
| | | | | | | | TLTISSLQPEDFATYYCQQSYSIP |
| | | | | | | | ITFGGGTKVEIK (SEQ ID NO: |
| | | | | | | | 245) |
| 37. | YTFS | GWMN | CARG | RASQSI | AASTLQS | CQQSYSTPWTF | QVQLVQSGAEVKKPGASVKVSCKA |
| | KHFV | PNSG | EGGY | SSWLA | (SEQ ID | (SEQ ID NO: | SGYTFSKHFVHWVRQAPGQGLEWM |
| | H | NSGY | YYYG | (SEQ | NO: | 114) | GWMNPNSGNSGYAQKFQGRVTMTR |
| | (SEQ | A | MDVW | ID NO: | 113) | | DTSTSTVYMELSSLRSEDTAVYYC |
| | ID | (SEQ | (SEQ | 196) | | | ARGEGGYYYYGMDVWGQGTLVTVS |
| | NO: | ID | ID | | | | SGGGGSGGGGSGGGGSGGGGSDIQ |
| | 246) | NO: | NO: | | | | MTQSPSSLSASVGDRVTITCRASQ |
| | | 247) | 248) | | | | SISSWLAWYQQKPGKAPKLLIYAA |
| | | | | | | | STLQSGVPSRFSGSGSGTDFTLTI |
| | | | | | | | SSLQPEDFATYYCQQSYSTPWTFG |
| | | | | | | | QGTKVEIK (SEQ ID NO: |
| | | | | | | | 249) |
| 38. | FTFG | SAIG | CAKG | RASQPL | AASSLQS | CQQAISFPLTF | EVQLLESGGGLVQPGGSLRLSCAA |
| | SYSM | TGGG | TPYY | SNWLA | (SEQ ID | (SEQ ID NO: | SGFTFGSYSMSWVRQAPGKGLEWV |
| | S | TYYA | YYYG | (SEQ | NO: 65) | 254) | SAIGTGGGTYYADSVKGRFTISRD |
| | (SEQ | (SEQ | MDVW | ID NO: | | | NSKNTLYLQMNSLRAEDTAVYYCA |
| | ID | ID | (SEQ | 253) | | | KGTPYYYYYGMDVWGQGTMVTVSS |
| | NO: | NO: | ID | | | | GGGGSGGGGSGGGGSGGGGSDIQM |
| | 250) | 251) | NO: | | | | TQSPSSLSASVGDRVTITCRASQP |
| | | | 252 | | | | LSNWLAWYQQKPGKAPKLLIYAAS |
| | | | | | | | SLQSGVPSRFSGSGSGTDFTLTIS |
| | | | | | | | SLQPEDFATYYCQQAISFPLTFGG |
| | | | | | | | GTKVEIK (SEQ ID NO: 255) |
| 39. | YTFT | GWMN | CARD | QSSEDI | AASSLQI | CQQTYSTPYTF | QVQLVQSGAEVKKPGASVKVSCKA |
| | SYYM | PNSG | LGYY | SSSLN | (SEQ ID | (SEQ ID NO: | SGYTFTSYYMHWVRQAPGQGLEWM |
| | H | NTGY | DSSG | (SEQ | NO: | 259) | GWMNPNSGNTGYAQKFQGRVTMTR |
| | (SEQ | A | YFGA | ID NO: | 258) | | DTSTSTVYMELSSLRSEDTAVYYC |
| | ID | (SEQ | FDIW | 257) | | | ARDLGYYDSSGYFGAFDIWGQGTT |
| | NO: | ID | (SEQ | | | | VTVSSGGGGSGGGGSGGGGSGGGG |
| | 215) | NO: | ID | | | | SDIQMTQSPSSLSASVGDRVTITC |
| | | 205) | NO: | | | | QSSEDISSSLNWYQQKPGKAPKLL |
| | | | 256) | | | | IYAASSLQIGVPSRFSGSGSGTDE |
| | | | | | | | TLTISSLQPEDFATYYCQQTYSTP |
| | | | | | | | YTFGQGTKVEIK (SEQ ID NO: |
| | | | | | | | 260) |
| 40. | YTFT | GIIN | CARG | RASQGI | AASNLET | CQQIHSYPLTF | QVQLVQSGAEVKKPGASVKVSCKA |
| | SYGI | PRGG | TRSS | GNWLA | (SEQ ID | (SEQ ID NO: | SGYTFTSYGISWVRQAPGQGLEWM |
| | S | STIF | GWYG | (SEQ | NO: | 265) | GIINPRGGSTIFAQKFQGRVTMTR |
| | (SEQ | A | WFDP | ID NO: | 264) | | DTSTSTVYMELSSLRSEDTAVYYC |
| | ID | (SEQ | W | 263) | | | ARGTRSSGWYGWFDPWGQGTLVTV |
| | NO: | ID | (SEQ | | | | SSGGGGSGGGGSGGGGSGGGGSDI |
| | 228) | NO: | ID | | | | QMTQSPSSLSASVGDRVTITCRAS |
| | | 261) | NO: | | | | QGIGNWLAWYQQKPGKAPKLLIYA |
| | | | 262) | | | | ASNLETGVPSRFSGSGSGTDFTLT |
| | | | | | | | ISSLQPEDFATYYCQQIHSYPLTF |
| | | | | | | | GGGTKVEIK (SEQ ID NO: |
| | | | | | | | 266) |
| 41. | FTFD | SYIS | CARE | RASQSI | AASSLQS | CQQSYSTPLTF | EVQLLESGGGLVQPGGSLRLSCAA |
| | DYGM | SSSS | IAAA | SSYLN | (SEQ ID | (SEQ ID NO: | SGFTFDDYGMSWVRQAPGKGLEWV |
| | S | YIYY | GFYG | (SEQ | NO: 65) | 173) | SYISSSSSYIYYADSVKGRFTISR |
| | (SEQ | A | MDVW | ID NO: | | | DNSKNTLYLQMNSLRAEDTAVYYC |
| | ID | (SEQ | (SEQ | 172) | | | AREIAAAGFYGMDVWGQGTTVTVS |
| | NO: | ID | ID | | | | SGGGGSGGGGSGGGGSGGGGSDIQ |
| | 267) | NO: | NO: | | | | MTQSPSSLSASVGDRVTITCRASQ |
| | | 268) | 269) | | | | SISSYLNWYQQKPGKAPKLLIYAA |
| | | | | | | | SSLQSGVPSRFSGSGSGTDFTLTI |
| | | | | | | | SSLQPEDFATYYCQQSYSTPLTFG |
| | | | | | | | GGTKVEIK (SEQ ID NO: |
| | | | | | | | 270) |
| 42. | GTLS | GGII | CARD | RASQSV | GASTRAT | CQQYGSSPITF | QVQLVQSGAEVKKPGSSVKVSCKA |
| | RYGV | PIFG | RVYY | SSSYLA | (SEQ ID | (SEQ ID NO: | SGGTLSRYGVSWVRQAPGQGLEWM |
| | S | TTNY | DSSG | (SEQ | NO: | 275) | GGIIPIFGTTNYAQKFQGRVTITA |
| | (SEQ | A | YPTW | ID NO: | 241) | | DESTSTAYMELSSLRSEDTAVYYC |
| | ID | (SEQ | YFDL | 274) | | | ARDRVYYDSSGYPTWYFDLWGRGT |
| | NO: | ID | W | | | | LVTVSSGGGGSGGGGSGGGGSGGG |
| | 271) | NO: | (SEQ | | | | GSEIVMTQSPATLSVSPGERATLS |
| | | 272 | ID | | | | CRASQSVSSSYLAWYQQKPGQAPR |
| | | | NO: | | | | LLIYGASTRATGIPARFSGSGSGT |
| | | | 273) | | | | EFTLTISSLQSEDFAVYYCQQYGS |
| | | | | | | | SPITFGQGTKVEIK (SEQ ID |
| | | | | | | | NO: 276) |
| 43. | FTFD | SGIS | CARD | QASQDI | KASTLES | CQQANSFPLTF | EVQLLESGGGLVQPGGSLRLSCAA |
| | DFAM | GNGD | ASYG | RNYLN | (SEQ ID | (SEQ ID NO: | SGFTFDDFAMHWVRQAPGKGLEWV |
| | H | SRYY | GNYG | (SEQ | NO: | 167) | SGISGNGDSRYYADSVKGRFTISR |
| | (SEQ | A | MDVW | ID NO: | 139) | | DNSKNTLYLQMNSLRAEDTAVYYC |
| | ID | (SEQ | (SEQ | 280) | | | ARDASYGGNYGMDVWGQGTTVTVS |
| | NO: | ID | ID | | | | SGGGGSGGGGSGGGGSGGGGSDIQ |
| | 277) | NO: | NO: | | | | MTQSPSSLSASVGDRVTITCQASQ |
| | | 278) | 279) | | | | DIRNYLNWYQQKPGKAPKLLIYKA |
| | | | | | | | STLESGVPSRFSGSGSGTDFTLTI |
| | | | | | | | SSLQPEDFATYYCQQANSFPLTFG |
| | | | | | | | PGTKVDIK (SEQ ID NO: |
| | | | | | | | 281) |
| 44. | FTQS | SAIG | CARE | RASQSI | GASNLQS | CQQSYSTPWTF | EVQLVESGGGLVKPGGSLRLSCAA |
| | SYWM | TGGG | WLVP | SRWLA | (SEQ ID | (SEQ ID NO: | SGFTFSSYWMSWVRQAPGKGLEWV |
| | S | TYYA | YYGM | (SEQ | NO: | 114) | SAIGTGGGTYYAAPVKGRFTISRD |
| | (SEQ | (SEQ | DVW | ID NO: | 284) | | DSKNTLYLQMNSLKTEDTAVYYCA |
| | ID | ID | (SEQ | 283) | | | REWLVPYYGMDVWGQGTTVTVSSG |
| | NO: | NO: | ID | | | | GGGSGGGGSGGGGSGGGGSDIQMT |
| | 169) | 251) | NO: | | | | QSPSSLSASVGDRVTITCRASQSI |
| | | | 282) | | | | SRWLAWYQQKPGKAPKLLIYGASN |
| | | | | | | | LQSGVPSRFSGSGSGTDFTLTISS |
| | | | | | | | LQPEDFATYYCQQSYSTPWTFGQG |
| | | | | | | | TKVEIK (SEQ ID NO: 285) |
| 45. | FSVS | AGIS | CARS | KSSQSV | WASTRQS | CHQYYGHPPTF | EVQLLESGGGLVQPGGSLRLSCAA |
| | SNYM | YDGS | RGIA | LYSSNN | (SEQ ID | (SEQ ID NO: | SGFSVSSNYMSWVRQAPGKGLEWV |
| | S | SKPY | ARPL | KNYLA | NO: | 291) | AGISYDGSSKPYADSVKGRFTISR |
| | (SEQ | A | QHW | (SEQ | 290) | | DNSKNTLYLQMNSLRAEDTAVYYC |
| | ID | (SEQ | (SEQ | ID NO: | | | ARSRGIAARPLQHWGQGTLVTVSS |
| | NO: | ID | ID | 289) | | | GGGGSGGGGSGGGGSGGGGSDIVM |
| | 286) | NO: | NO: | | | | TQSPDSLAVSLGERATINCKSSQS |
| | | 287) | 288) | | | | VLYSSNNKNYLAWYQQKPGQPPKL |
| | | | | | | | LIYWASTRQSGVPDRFSGSGSGTD |
| | | | | | | | FTLTISSLQAEDVAVYYCHQYYGH |
| | | | | | | | PPTFGGGTKVEIK (SEQ ID |
| | | | | | | | NO: 292) |
| 46. | FSVS | AGIS | CARS | KSSQSV | QASTRQS | CHQYYGHPPTF | EVQLLESGGGLVQPGGSLRLSCAA |
| | SNYM | YDGS | RGIA | LYSSNN | (SEQ ID | (SEQ ID NO: | SGFSVSSNYMSWVRQAPGKGLEWV |
| | S | SKPY | ARPL | KNYLA | NO: | 291) | AGISYDGSSKPYADSVKGRFTISR |
| | (SEQ | A | QHW | (SEQ | 293) | | DNSKNTLYLQMNSLRAEDTAVYYC |
| | ID | (SEQ | (SEQ | ID NO: | | | ARSRGIAARPLQHWGQGTLVTVSS |
| | NO: | ID | ID | 289) | | | GGGGSGGGGSGGGGSGGGGSDIVM |
| | 286) | NO: | NO: | | | | TQSPDSLAVSLGERATINCKSSQS |
| | | 287) | 288) | | | | VLYSSNNKNYLAWYQQKPGQPPKL |
| | | | | | | | LIYQASTRQSGVPDRFSGSGSGTD |
| | | | | | | | FTLTISSLQAEDVAVYYCHQYYGH |
| | | | | | | | PPTFGGGTKVEIK (SEQ ID |
| | | | | | | | NO: 294) |
| 47. | FSFS | SAIS | CARD | RASQGI | DASNLET | CQQSYSTPLTF | EVQLLESGGGLVQPGGSLRLSCAA |
| | DYGM | GSGG | GGWQ | SNNLN | (SEQ ID | (SEQ ID NO: | SGFSFSDYGMHWVRQAPGKGLEWV |
| | H | STYY | PAAI | (SEQ | NO: | 173) | SAISGSGGSTYYADSVKGRFTISR |
| | (SEQ | A | LDYW | ID NO: | 159) | | DNSKNTLYLQMNSLRAEDTAVYYC |
| | ID | (SEQ | (SEQ | 105) | | | ARDGGWQPAAILDYWGQGTLVTVS |
| | NO: | ID | ID | | | | SGGGGSGGGGSGGGGSGGGGSDIQ |
| | 295) | NO: | NO: | | | | MTQSPSSLSASVGDRVTITCRASQ |
| | | 103) | 296) | | | | GISNNLNWYQQKPGKAPKLLIYDA |
| | | | | | | | SNLETGVPSRFSGSGSGTDFTLTI |
| | | | | | | | SSLQPEDFATYYCQQSYSTPLTFG |
| | | | | | | | GGTKVEIK (SEQ ID NO: |
| | | | | | | | 297) |
| 48. | FTFS | SVIY | CARD | RASQGI | DASNLET | CFQSYSTCYTF | EVQLLESGGGLVQPGGSLRLSCAA |
| | DHGM | GGES | PAVA | SNYLA | (SEQ ID | (SEQ ID NO: | SGFTFSDHGMHWVRQAPGKGLEWV |
| | H | TYYA | GGGI | (SEQ | NO: | 301) | SVIYGGESTYYADSVKGRFTISRD |
| | (SEQ | (SEQ | FDYW | ID NO: | 159) | | NSKNTLYLQMNSLRAEDTAVYYCA |
| | ID | ID | (SEQ | 218) | | | RDPAVAGGGIFDYWGQGTLVTVSS |
| | NO: | NO: | ID | | | | GGGGSGGGGSGGGGSGGGGSDIQM |
| | 298) | 299) | NO: | | | | TQSPSSLSASVGDRVTITCRASQG |
| | | | 300) | | | | ISNYLAWYQQKPGKAPKLLIYDAS |
| | | | | | | | NLETGVPSRFSGSGSGTDFTLTIS |
| | | | | | | | SLQPEDFATYYCQQSYSTCYTFGQ |
| | | | | | | | GTKLEIK (SEQ ID NO: 302) |
| 49. | DTFT | GWIN | CARS | RASQTI | DASTLFS | CFQYSSYPLTF | QVQLVQSGAEVKKPGASVKVSCKA |
| | GYYI | PNSG | GLWL | SIWLA | (SEQ ID | (SEQ ID NO: | SGDTFTGYYIHWVRQAPGQGLEWM |
| | H | GTNY | GSYY | (SEQ | NO: | 308) | GWINPNSGGTNYAQKFQGRVTMTR |
| | (SEQ | A | GMDV | ID NO: | 307) | | DTSTSTVYMELSSLRSEDTAVYYC |
| | ID | (SEQ | W | 306) | | | ARSGLWLGSYYGMDVWGQGTLVTV |
| | NO: | ID | (SEQ | | | | SSGGGGSGGGGSGGGGSGGGGSDI |
| | 303) | NO: | ID | | | | QMTQSPSSLSASVGDRVTITCRAS |
| | | 304) | NO: | | | | QTISIWLAWYQQKPGKAPKLLIYD |
| | | | 305) | | | | ASTLQSGVPSRFSGSGSGTDFTLT |
| | | | | | | | ISSLQPEDFATYYCQQYSSYPLTF |
| | | | | | | | GQGTKVEIK (SEQ ID NO: |
| | | | | | | | 309) |
| 50. | YTFT | GWIN | CARS | RASHFI | AASTLFS | CQQSYSGISF | QVQLVQSGAEVKKPGASVKVSCKA |
| | SYDI | PNSG | PYYY | SRWVA | (SEQ ID | (SEQ ID NO: | SGYTFTSYDINWVRQAPGQGLEWM |
| | N | TTGY | YGMD | (SEQ | NO: | 314) | GWINPNSGTTGYAQKFQGRVTMTR |
| | (SEQ | A | VW | ID NO: | 113) | | DTSTSTVYMELSSLRSEDTAVYYC |
| | ID | (SEQ | (SEQ | 313) | | | ARSPYYYYGMDVWGQGTTVTVSSG |
| | NO: | ID | ID | | | | GGGSGGGGSGGGGSGGGGSDIQMT |
| | 310) | NO: | NO: | | | | QSPSSLSASVGDRVTITCRASHFI |
| | | 311) | 312) | | | | SRWVAWYQQKPGKAPKLLIYAAST |
| | | | | | | | LQSGVPSRFSGSGSGTDFTLTISS |
| | | | | | | | LQPEDFATYYCQQSYSGISFGPGT |
| | | | | | | | KVDIK (SEQ ID NO: 315) |
| 51. | FTFN | SRIN | CARG | RASQSV | ATSSRAS | CQQYYSGLTF | EVQLLESGGGLVQPGGSLRLSCAA |
| | NYGM | SDGS | AYYY | SGSYLA | (SEQ ID | (SEQ ID NO: | SGFTFNNYGMNWVRQAPGKGLEWV |
| | N | STSY | YYMD | (SEQ | NO: | 321) | SRINSDGSSTSYADSVKGRFTISR |
| | (SEQ | A | VW | ID NO: | 320) | | DNSKNTLYLQMNSLRAEDTAVYYC |
| | ID | (SEQ | (SEQ | 319) | | | ARGAYYYYYMDVWGQGTLVTVSSG |
| | NO: | ID | ID | | | | GGGSGGGGSGGGGSGGGGSEIVMT |
| | 316) | NO: | NO: | | | | QSPATLSVSPGERATLSCRASQSV |
| | | 317) | 318) | | | | SGSYLAWYQQKPGQAPRLLIYATS |
| | | | | | | | SRASGIPARFSGSGSGTEFTLTIS |
| | | | | | | | SLQSEDFAVYYCQQYYSGLTFGQG |
| | | | | | | | TKVEIK (SEQ ID NO: 322) |
| 52. | FTFS | AHIW | CARD | RASQDI | DASSLET | CQQATSLPLTF | EVQLLESGGGLVQPGGSLRLSCAA |
| | NSDM | NDGS | RTDP | RNYLG | (SEQ ID | (SEQ ID NO: | SGFTFSNSDMNWVRQAPGKGLEWV |
| | N | QKYY | GYSS | (SEQ | NO: | 328) | AHIWNDGSQKYYADSVKGRFTISR |
| | (SEQ | A | AMDV | ID NO: | 327) | | DNSKNTLYLQMNSLRAEDTAVYYC |
| | ID | (SEQ | W | 326) | | | ARDRTDPGYSSAMDVWGQGTTVTV |
| | NO: | ID | (SEQ | | | | SSGGGGSGGGGSGGGGSGGGGSDI |
| | 323) | NO: | ID | | | | QMTQSPSSLSASVGDRVTITCRAS |
| | | 324) | NO: | | | | QDIRNYLGWYQQKPGKAPKLLIYD |
| | | | 325) | | | | ASSLETGVPSRFSGSGSGTDFTLT |
| | | | | | | | ISSLQPEDFATYYCQQATSLPLTF |
| | | | | | | | GGGTKVEIK (SEQ ID NO: |
| | | | | | | | 329) |
| 53. | YTFT | GWMN | CAKD | RASQDI | QASSLES | CQQSYTIPLTF | QVQLVQSGAEVKKPGASVKVSCKA |
| | SYDI | PNSG | SDYS | TNDLG | (SEQ ID | (SEQ ID NO: | SGYTFTSYDINWVRQAPGQGLEWM |
| | N | NTGY | NLLW | (SEQ | NO: | 333) | GWMNPNSGNTGYAQKFQGRVTMTR |
| | (SEQ | A | DYW | ID NO: | 332) | | DTSTSTVYMELSSLRSEDTAVYYC |
| | ID | (SEQ | (SEQ | 331) | | | AKDSDYSNLLWDYWGQGTLVTVSS |
| | NO: | ID | ID | | | | GGGGSGGGGSGGGGSGGGGSDIQM |
| | 310) | NO: | NO: | | | | TQSPSSLSASVGDRVTITCRASQD |
| | | 205) | 330) | | | | ITNDLGWYQQKPGKAPKLLIYQAS |
| | | | | | | | SLESGVPSRFSGSGSGTDFTLTIS |
| | | | | | | | SLQPEDFATYYCQQSYTIPLTFGQ |
| | | | | | | | GTKVEIK (SEQ ID NO: 334) |
| 54. | FTFG | AVVS | CAKD | RASQNI | DASNLET | CQQANSFPPTF | EVQLLESGGGLVQPGGSLRLSCAA |
| | DYAM | YDGT | ICSS | NNYVN | (SEQ ID | (SEQ ID NO: | SGFTFGDYAMSWVRQAPGKGLEWV |
| | S | NKYY | TSCY | (SEQ | NO: | 338) | AVVSYDGTNKYYADSVKGRFTISR |
| | (SEQ | A | FDLW | ID NO: | 159) | | DNSKNTLYLQMNSLRAEDTAVYYC |
| | ID | (SEQ | (SEQ | 337) | | | AKDICSSTSCYFDLWGRGTLVTVS |
| | NO: | ID | ID | | | | SGGGGSGGGGSGGGGSGGGGSDIQ |
| | 233) | NO: | NO: | | | | MTQSPSSLSASVGDRVTITCRASQ |
| | | 335) | 336) | | | | NINNYVNWYQQKPGKAPKLLIYDA |
| | | | | | | | SNLETGVPSRFSGSGSGTDFTLTI |
| | | | | | | | SSLQPEDFATYYCQQANSFPPTFG |
| | | | | | | | QGTRLEIK (SEQ ID NO: |
| | | | | | | | 339) |
| 55. | YTFT | GIID | CARE | RASQGI | ATSSLQT | CQQTYSIPITF | QVQLVQSGAEVKKPGASVKVSCKA |
| | SYYM | PSGG | EWSS | SSYLA | (SEQ ID | SEQ ID NO: | SGYTFTSYYMHWVRQAPGQGLEWM |
| | H | STSY | GGVG | (SEQ | NO: | 344) | GIIDPSGGSTSYAQKFQGRVTMTR |
| | (SEQ | A | YFDY | ID NO: | 343) | | DTSTSTVYMELSSLRSEDTAVYYC |
| | ID | (SEQ | W | 342) | | | AREEWSSGGVGYFDYWGQGTLVTV |
| | NO: | ID | (SEQ | | | | SSGGGGSGGGGSGGGGSGGGGSDI |
| | 215) | NO: | ID | | | | QMTQSPSSLSASVGDRVTITCRAS |
| | | 340) | NO: | | | | QGISSYLAWYQQKPGKAPKLLIYA |
| | | | 341) | | | | TSSLQTGVPSRFSGSGSGTDFTLT |
| | | | | | | | ISSLQPEDFATYYCQQTYSIPITF |
| | | | | | | | GQGTRLEIK (SEQ ID NO: |
| | | | | | | | 345) |
| 56. | FTFD | SAIS | CARD | QASQDI | KASSLES | CQQANSYPVTF | EVQLLESGGGLVQPGGSLRLSCAA |
| | DYAM | GGGE | ASYG | RNYLN | (SEQ ID | (SEQ ID NO: | SGFTFDDYAMHWVRQAPGKGLEWV |
| | H | DTYY | GNYG | (SEQ | NO: | 348) | SAISGGGEDTYYADSVKGRFTISR |
| | (SEQ | A | MDVW | ID NO: | 347) | | DNSKNTLYLQMNSLRAEDTAVYYC |
| | ID | (SEQ | (SEQ | 280) | | | ARDASYGGNYGMDVWGQGTTVTVS |
| | NO: | ID | ID | | | | SGGGGSGGGGSGGGGSGGGGSDIQ |
| | 135) | NO: | NO: | | | | MTQSPSSLSASVGDRVTITCQASQ |
| | | 346) | 279) | | | | DIRNYLNWYQQKPGKAPKLLIYKA |
| | | | | | | | SSLESGVPSRFSGSGSGTDFTLTI |
| | | | | | | | SSLQPEDFATYYCQQANSYPVTFG |
| | | | | | | | GGTKVEIK (SEQ ID NO: |
| | | | | | | | 349) |
| 57. | YTFT | GIIN | CARD | RASQGI | AASSLQG | CQQSYSLPYTF | QVQLVQSGAEVKKPGASVKVSCKA |
| | SYYM | PSGG | SVAG | SNYFA | (SEQ ID | (SEQ ID NO: | SGYTFTSYYMHWVRQAPGQGLEWM |
| | H | STSY | TGGR | (SEQ | NO: | 353) | GIINPSGGSTSYAQKFQGRVTMTR |
| | (SEQ | A | YYGM | ID NO: | 352) | | DTSTSTVYMELSSLRSEDTAVYYC |
| | ID | (SEQ | DVW | 351) | | | ARDSVAGTGGRYYGMDVWGQGTLV |
| | NO: | ID | (SEQ | | | | TVSSGGGGSGGGGSGGGGSGGGGS |
| | 215) | NO: | ID | | | | DIQMTQSPSSLSASVGDRVTITCR |
| | | 69) | NO: | | | | ASQGISNYFAWYQQKPGKAPKLLI |
| | | | 350) | | | | YAASSLQGGVPSRFSGSGSGTDFT |
| | | | | | | | LTISSLQPEDFATYYCQQSYSLPY |
| | | | | | | | TFGQGTKLEIK (SEQ ID NO: |
| | | | | | | | 354) |
| 58. | YTFT | GIIN | CTTA | RASQGI | AASSLQS | CQQYYSNADF | QVQLVQSGAEVKKPGASVKVSCKA |
| | GYYM | PSGG | DYYY | SNYLA | (SEQ ID | (SEQ ID NO: | SGYTFTGYYMHWVRQAPGQGLEWM |
| | H | NTKY | YMDV | (SEQ | NO: 65) | 357) | GIINPSGGNTKYAQKFQGRVTMTR |
| | (SEQ | A | W | ID NO: | | | DTSTSTVYMELSSLRSEDTAVYYC |
| | ID | (SEQ | (SEQ | 218) | | | TTADYYYYMDVWGKGTTVTVSSGG |
| | NO: | ID | ID | | | | GGSGGGGSGGGGSGGGGSDIQMTQ |
| | 128) | NO: | NO: | | | | SPSSLSASVGDRVTITCRASQGIS |
| | | 355) | 356) | | | | NYLAWYQQKPGKAPKLLIYAASSL |
| | | | | | | | QSGVPSRFSGSGSGTDFTLTISSL |
| | | | | | | | QPEDFATYYCQQYYSNADFGQGTK |
| | | | | | | | VEIK (SEQ ID NO: 358) |
| 59. | FTFS | SYIS | CARD | RASQSV | SSLQS | QQYKSYPVT | EVQLLESGGGLVQPGGSLRLSCAA |
| | DFWM | GDSG | RPYY | SRSLA | (SEQ ID | (SEQ ID NO: | SGFTFSDFWMHWVRQAPGKGLEWI |
| | H | YTNY | YYMD | (SEQ | NO: | 363) | SYISGDSGYTNYADSVKGRFTISR |
| | (SEQ | A | VW | ID NO: | 362) | | DNSKNTLYLQMNSLRAEDTAVYYC |
| | ID | (SEQ | (SEQ | 361) | | | ARDRPYYYYMDVWGKGTTVTVSSG |
| | NO: | ID | ID | | | | GGGSGGGGSGGGGSGGGGSDIQMT |
| | 359) | NO: | NO: | | | | QSPSSLSASVGDRVTITCRASQSV |
| | | 170) | 360) | | | | SRSLAWYQQKPGKAPKLLIYAASS |
| | | | | | | | LQSGVPSRFSGSGSGTDFTLTISS |
| | | | | | | | LQPEDFATYYCQQYKSYPVTFGQG |
| | | | | | | | TKVEIK (SEQ ID NO: 364) |
| 60. | FTFD | SDIS | CAKD | QASQDI | SYLQS | QQAHNYPIT | EVQLLESGGGLVQPGGSLRLSCAA |
| | DYTM | GSGG | VVVA | SNYLN | (SEQ ID | (SEQ ID NO: | SGFTFDDYTMHWVRQAPGKGLEWV |
| | H | STYY | GTPL | (SEQ | NO: | 369) | SDISGSGGSTYYADSVKGRFTISR |
| | (SEQ | A | HFDY | ID NO: | 368) | | DNSKNTLYLQMNSLRAEDTAVYYC |
| | ID | (SEQ | W | 138) | | | AKDVVVAGTPLHFDYWGQGTLVTV |
| | NO: | ID | (SEQ | | | | SSGGGGSGGGGSGGGGSGGGGSDI |
| | 365) | NO: | ID | | | | QMTQSPSSLSASVGDRVTITCQAS |
| | | 366) | NO: | | | | QDISNYLNWYQQKPGKAPKLLIYA |
| | | | 367) | | | | ASYLQSGVPSRFSGSGSGTDFTLT |
| | | | | | | | ISSLQPEDFATYYCQQAHNYPITF |
| | | | | | | | GQGTRLEIK (SEQ ID NO: |
| | | | | | | | 370) |
| 61. | FTFS | ASIS | CARE | RASQSI | SSLQS | QQANAFPPT | EVQLLESGGGLVQPGGSLRLSCAA |
| | NAWM | STSA | VVGA | STWLA | (SEQ ID | (SEQ ID NO: | SEFTFSNAWMSWVRQAPGKGLEWV |
| | S | YIDY | TTFD | (SEQ | NO: | 374) | ASISSTSAYIDYADSVKGRFTISR |
| | (SEQ | A | YW | ID NO: | 362) | | DNSKNTLYLQMNSLRAEDTAVYYC |
| | ID | (SEQ | (SEQ | 373) | | | AREVVGATTFDYWGQGTLVTVSSG |
| | NO: | ID | ID | | | | GGGSGGGGSGGGGSGGGGSDIQMT |
| | 183) | NO: | NO: | | | | QSPSSLSASVGDRVTITCRASQSI |
| | | 371) | 372) | | | | STWLAWYQQKPGKAPKLLIYAASS |
| | | | | | | | LQSGVPSRFSGSGSGTDFTLTISS |
| | | | | | | | LQPEDFATYYCQQANAFPPTFGQG |
| | | | | | | | TRLEIK (SEQ ID NO: 375) |
| 62. | GTFS | GWME | CAKG | KSSQSV | STRES | QQYYSTPPT | QVQLVQSGAEVKKPGSSVKVSCKA |
| | SYAI | PHTG | GFSW | LYSSNN | (SEQ ID | (SEQ ID NO: | SGGTFSSYAISWVRQAPGQGLEWM |
| | S | NTRY | FDPW | KNYLA | NO: | 379) | GWMEPHTGNTRYAQKFQGRVTITA |
| | (SEQ | A | (SEQ | (SEQ | 378) | | DESTSTAYMELSSLRSEDTAVYYC |
| | ID | (SEQ | ID | ID NO: | | | AKGGFSWFDPWGQGTLVTVSSGGG |
| | NO: | ID | NO: | 289) | | | GSGGGGSGGGGSGGGGSDIVMTQS |
| | 77) | NO: | 377) | | | | PDSLAVSLGERATINCKSSQSVLY |
| | | 376) | | | | | SSNNKNYLAWYQQKPGQPPKLLIY |
| | | | | | | | WASTRESGVPDRFSGSGSGTDFTL |
| | | | | | | | TISSLQAEDVAVYYCQQYYSTPPT |
| | | | | | | | FGQGTRLEIK (SEQ ID NO: |
| | | | | | | | 380) |
| 63. | FTFD | ASIT | CARE | RASQGI | STRAT | QQYYTYPPT | EVQLLESGGGLVKPGGSLRLSCAA |
| | DYAM | SSSA | RVDW | SNSYLA | (SEQ ID | (SEQ ID NO: | SGFTFDDYAMHWVRQAPGKGLEWV |
| | H | FIDY | NSYF | (SEQ | NO: | 385) | ASITSSSAFIDYAASVKGRFTISR |
| | (SEQ | A | DLW | ID NO: | 384) | | DDSKNTLYLQMNSLKTEDTAVYYC |
| | ID | (SEQ | (SEQ | 383) | | | ARERVDWNSYFDLWGRGTLVTVSS |
| | NO: | ID | ID | | | | GGGGSGGGGSGGGGSGGGGSEIVM |
| | 135) | NO: | NO: | | | | TQSPATLSVSPGERATLSCRASQG |
| | | 381) | 382) | | | | ISNSYLAWYQQKPGQAPRLLIYGA |
| | | | | | | | STRATGIPARFSGSGSGTEFTLTI |
| | | | | | | | SSLQSEDFAVYYCQQYYTYPPTFG |
| | | | | | | | PGTKVDIK (SEQ ID NO: |
| | | | | | | | 386) |
| 64. | FAFS | AGTS | CARE | RASQGI | ANLEG | QQSDIFPPT | EVQLLESGGGLVKPGGSLRLSCAA |
| | SHWM | GSGE | TYYY | SNYLA | (SEQ ID | (SEQ ID NO: | SGFAFSSHWMHWVRQAPGKGLEWV |
| | H | SRDY | YYMD | (SEQ | NO: | 391) | AGTSGSGESRDYADFVKGRFTISR |
| | (SEQ | A | VW | ID NO: | 390) | | DDSKNTLYLQMNSLKTEDTAVYYC |
| | ID | (SEQ | (SEQ | 218) | | | ARETYYYYYMDVWGKGTTVTVSSG |
| | NO: | ID | ID | | | | GGGSGGGGSGGGGSGGGGSDIQMT |
| | 387) | NO: | NO: | | | | QSPSSLSASVGDRVTITCRASQGI |
| | | 388) | 389) | | | | SNYLAWYQQKPGKAPKLLIYDAAN |
| | | | | | | | LEGGVPSRFSGSGSGTDFTLTISS |
| | | | | | | | LQPEDFATYYCQQSDIFPPTFGQG |
| | | | | | | | TKVEIK (SEQ ID NO: 392) |
| 65. | YTFT | GWIN | CARE | RASQSI | SSLQS | QQSNSFPLT | QVQLVQSGAEVKKPGASVKVSCKA |
| | RHWI | VKTG | SSGW | SNYLA | (SEQ ID | (SEQ ID NO: | SGYTFTRHWIHWVRQAPGQGLEWM |
| | H | GAGY | YGTD | (SEQ | NO: | 397) | GWINVKTGGAGYAQKFQGRVTMTR |
| | (SEQ | A | VW | ID NO: | 362) | | DTSTSTVYMELSSLRSEDTAVYYC |
| | ID | (SEQ | (SEQ | 396) | | | ARESSGWYGTDVWGQGTTVTVSSG |
| | NO: | ID | ID | | | | GGGSGGGGSGGGGSGGGGSDIQMT |
| | 393) | NO: | NO: | | | | QSPSSLSASVGDRATITCRASQSI |
| | | 394) | 395) | | | | SNYLAWYQQKPGKAPKLLIYAASS |
| | | | | | | | LQSGVPSRFSGSGSGTDFTLTISS |
| | | | | | | | LQPEDFATYYCQQSNSFPLTFGGG |
| | | | | | | | TKVEIK (SEQ ID NO: 398) |
| 66. | FTFS | AAIS | CARE | QASQDI | NLRS | QQANSFPVT | EVQLLESGGGLVQPGGSLRLSCAA |
| | SYWM | YDGK | NKQW | SNFVN | (SEQ ID | (SEQ ID NO: | SGFTFSSYWMHWVRQAPGKGLEWV |
| | H | YKDY | LASF | (SEQ | NO: | 403) | AAISYDGKYKDYEDSVKGRFTISR |
| | (SEQ | E | DYW | ID NO: | 402) | | DNSKNTLYLQMNSLRAEDTAVYYC |
| | ID | (SEQ | (SEQ | 401) | | | ARENKQWLASFDYWGQGTLVTVSS |
| | NO: | ID | ID | | | | GGGGSGGGGSGGGGSGGGGSDIQM |
| | 83) | NO: | NO: | | | | TQSPSSLSASVGDRVTITCQASQD |
| | | 399) | 400) | | | | ISNFVNWYQQKPGKAPKLLIYAAN |
| | | | | | | | LRSGVPSRFSGSGSGTDFTLTISS |
| | | | | | | | LQPEDFATYYCQQANSFPVTFGPG |
| | | | | | | | TKVDIK (SEQ ID NO: 404) |

In some embodiments, the antibody comprises a CDR set as set forth in Table 6 or Table 7. In some embodiments, the antibody comprises the CDRs of Clone ID: 6, Clone ID: 59, or Clone ID: 63 of Table 6.

The antibodies, can be in a scFv format, which are also illustrated in a non-limiting embodiment in Table 6.

In some embodiments, the MAdCAM antibody is selected from the following table, which can be in a IgG format as illustrated in Table 7.

TABLE 7

| Clone | | | | | | | | |
|---|---|---|---|---|---|---|---|---|
| ID | HCDR1 | HCDR2 | HCDR3 | LCDR1 | LCDR2 | LCDR3 | VH | VK |
| 1. | FTFSS | AVISD | CTTSK | QASQD | AASSL | CQQGY | EVQLLESGGGLVQP | DIQMTQSPSSLSA |
| | YGMH | DGSDK | YYYYY | ISKSL | QS (SE | STPLT | GGSLRLSCAASGFT | SVGDRVTITCQAS |
| | (SEQ | YYA | GMDVW | N | Q ID | F | FSSYGMHWVRQAPG | QDISKSLNWYQQK |
| | ID | (SEQ | (SEQ | (SEQ | NO: | (SEQ | KGLEWVAVISDDGS | PGKAPKLLIYAAS |
| | NO: | ID | ID | ID | 65) | ID | DKYYADSVKGRFTI | SLQSGVPSRESGS |
| | 61) | NO: | NO: | NO: | | NO: | SRDNSKNTLYLQMN | GSGTDFTLTISSL |
| | | 62) | 63) | 64) | | 66) | SLRAEDTAVYYCTT | QPEDFATYYCQQG |
| | | | | | | | SKYYYYYGMDVWGQ | YSTPLTFGGGTKV |
| | | | | | | | GTTVTVSS (SEQ | EIK (SEQ ID |
| | | | | | | | ID NO: 405) | NO: 406) |
| 2. | YPFIG | GIINP | CAREG | RASQS | GASTL | CQQTW | QVQLVQSGAEVKKP | DIQMTQSPSSLSA |
| | YYLH | SGGST | RLSYG | ISSYL | ES | GPPFT | GASVKVSCKASGYP | SVGDRVTITCRAS |
| | (SEQ | SYA | MDAW | A | (SEQ | F | FIGYYLHWVRQAPG | QSISSYLAWYQQK |
| | ID | (SEQ | (SEQ | (SEQ | ID | (SEQ | QGLEWMGIINPSGG | PGKAPKLLIYGAS |
| | NO: | ID | ID | ID | NO: | ID | STSYAQKFQGRVTM | TLESGVPSRFSGS |
| | 68) | NO: | NO: | NO: | 72) | NO: | TRDTSTSTVYMELS | GSGTDFTLTISSL |
| | | 69) | 70) | 71) | | 73) | SLRSEDTAVYYCAR | QPEDFATYYCQQT |
| | | | | | | | EGRLSYGMDAWGQG | WGPPFTFGQGTKL |
| | | | | | | | TLVTVSS (SEQ | EIK (SEQ ID |
| | | | | | | | ID NO: 407) | NO: 408) |
| 3. | YPFIG | GIINP | CAREG | RASQS | GASTL | CQQTW | QVQLVQSGAEVKKP | DIQMTQSPSSLSA |
| | QYLH | SGGST | RLSYG | ISSYL | ES | GPPFT | GASVKVSCKASGYP | SVGDRVTITCRAS |
| | (SEQ | SYA | MDAW | A | (SEQ | F | FIGQYLHWVRQAPG | QSISSYLAWYQQK |
| | ID | (SEQ | (SEQ | (SEQ | ID | (SEQ | QGLEWMGIINPSGG | PGKAPKLLIYGAS |
| | NO: | ID | ID | ID | NO: | ID | STSYAQKFQGRVTM | TLESGVPSRFSGS |
| | 75) | NO: | NO: | NO: | 72) | NO: | TRDTSTSTVYMELS | GSGTDFTLTISSL |
| | | 69) | 70) | 71) | | 73) | SLRSEDTAVYYCAR | QPEDFATYYCQQT |
| | | | | | | | EGRLSYGMDAWGQG | WGPPFTFGQGTKL |
| | | | | | | | TLVTVSS (SEQ | EIK (SEQ ID |
| | | | | | | | ID NO: 409) | NO: 408) |
| 4. | GTFSS | GSINP | CAKDK | QASQD | AASSL | CQQSY | QVQLVQSGAEVKKP | DIQMTQSPSSLSA |
| | YAIS | SGDTT | AQWLV | ISNSL | QS | SSVIT | GASVKVSCKASGGT | SVGDRVTITCQAS |
| | (SEQ | SYA | GYFDY | N | (SEQ | F | FSSYAISWVRQAPG | QDISNSLNWYQQK |
| | ID | (SEQ | W | (SEQ | ID | (SEQ | QGLEWMGSINPSGD | PGKAPKLLIYAAS |
| | NO: | ID | (SEQ | ID | NO: | ID | TTSYAQKFQGRVTM | SLQSGVPSRFSGS |
| | 77) | NO: | ID | NO: | 65) | NO: | TRDTSTSTVYMELS | GSGTDFTLTISSL |
| | | 78) | NO: | 80) | | 81) | SLRSEDTAVYYCAK | QPEDFATYYCQQS |
| | | | 79) | | | | DKAQWLVGYFDYWG | YSSVITFGQGTKV |
| | | | | | | | QGTLVTVSS (SEQ | EIK (SEQ ID |
| | | | | | | | ID NO: 410) | NO: 411) |
| 5. | FTFSS | SSISP | CAREV | RASQG | GASSL | CQQAN | EVQLLESGGGLVQP | DIQMTQSPSSLSA |
| | YWMH | GGSNI | QLSHY | ISNSL | QS | SFPFT | GGSLRLSCAASGFT | SVGDRVTITCRAS |
| | (SEQ | DYA | DYW | A | (SEQ | F | FSSYWMHWVRQAPG | QGISNSLAWYQQK |
| | ID | (SEQ | (SEQ | (SEQ | ID | (SEQ | KGLEWVSSISPGGS | PGKAPKLLIYGAS |
| | NO: | ID | ID | ID | NO: | ID | NIDYADSVKGRFTI | SLQSGVPSRFSGS |
| | 83) | NO: | NO: | NO: | 87) | NO: | SRDNSKNTLYLQMN | GSGTDFTLTISSL |
| | | 84) | 85) | 86) | | 88) | SLRAEDTAVYYCAR | QPEDFATYYCQQA |
| | | | | | | | EVQLSHYDYWGQGT | NSFPFTFGQGTKV |
| | | | | | | | LVTVSS (SEQ ID | EIK (SEQ ID |
| | | | | | | | NO: 412) | NO: 413) |
| 6. | FTFNN | SRINS | CAREG | RASQI | GASSL | CQQSY | EVQLLESGGGLVQP | DIQMTQSPSSLSA |
| | YAFH | YGTST | PVAGY | IGTNL | QS | RLPFT | GGSLRLSCAASGFT | SVGDRVTITCRAS |
| | (SEQ | TYA | WYFDL | A | (SEQ | F | FNNYAFHWVRQAPG | QIIGTNLAWYQQK |
| | ID | (SEQ | W | (SEQ | ID | SEQ | KGLEWVSRINSYGT | PGKAPKLLIYGAS |
| | NO: | ID | (SEQ | ID | NO: | ID | STTYADSVKGRFTI | SLQSGVPSRESGS |
| | 90) | NO: | ID | NO: | 87) | NO: | SRDNSKNTLYLQMN | GSGTDFTLTISSL |
| | | 91) | NO: | 93) | | 94) | SLRAEDTAVYYCAR | QPEDFATYYCQQS |
| | | | 92) | | | | EGPVAGYWYFDLWG | YRLPFTFGQGTKV |
| | | | | | | | QGTLVTVSS (SEQ | EIK (SEQ ID |
| | | | | | | | ID NO: 414) | NO: 415) |
| 7. | FTFSD | AIISH | CAKPY | RASRG | STLQS | QQAYS | EVQLLESGGGLVQP | DIQMTQSPSSLSA |
| | YQMS | ADGGF | SSGWS | ITNDL | (SEQ | FPWT | GGSLRLSCAASGFT | SVGDRVTITCRAS |
| | (SEQ | KDYA | AVYYF | G | ID | (SEQ | FSDYQMSWVRQAPG | RGITNDLGWYQQK |
| | ID | (SEQ | DYW | (SEQ | NO: | ID | KGLEWVAIISHADG | PGKAPKLLIYAAS |
| | NO: | ID | (SEQ | ID | 420) | NO: | GFKDYADSVKGRFT | TLQSGVPSRFSGS |
| | 416) | NO: | ID | NO: | | 421) | ISRDNSKNTLYLQM | GSGTDFTLTISSL |
| | | 417) | NO: | 419) | | | NSLRAEDTAVYYCA | QPEDFATYYCQQA |
| | | | 418) | | | | KPYSSGWSAVYYFD | YSFPWTFGQGTKV |
| | | | | | | | YWGQGTLVTVSS | EIK (SEQ ID |
| | | | | | | | (SEQ ID NO: | NO: 423) |
| | | | | | | | 422) | |
| 8. | YTFTG | GIINP | CAKDW | RASQN | AASSL | CQQSY | QVQLVQSGAEVKKP | DIQMTQSPSSLSA |
| | YHIH | SGGST | SSWYL | ISSSL | QS | TTPYT | GASVKVSCKASGYT | SVGDRVTITCRAS |
| | (SEQ | IYA | GPFDY | N | (SEQ | F | FTGYHIHWVRQAPG | QNISSSLNWYQQK |
| | ID | (SEQ | W | (SEQ | ID | (SEQ | QGLEWMGIINPSGG | PGKAPKLLIYAAS |
| | NO: | ID | (SEQ | ID | NO: | ID | STIYAQKFQGRVTM | SLQSGVPSRFSGS |
| | 96) | NO: | ID | NO: | 65) | NO: | TRDTSTSTVYMELS | GSGTDFTLTISSL |
| | | 97) | NO: | 99) | | 100) | SLRSEDTAVYYCAK | QPEDFATYYCQQS |
| | | | 98) | | | | DWSSWYLGPFDYWG | YTTPYTFGQGTKV |
| | | | | | | | QGTLVTVSS (SEQ | EIK (SEQ ID |
| | | | | | | | ID NO: 424) | NO: 425) |
| 9. | YTFTS | GIINH | CARPY | RASQS | STLQS | QQSYS | QVQLVQSGAEVKKP | DIQMTQSPSSLSA |
| | YYMH | SGGST | SGWYF | ISSSL | (SEQ | TPLT | GASVKVSCKASGYT | SVGDRVTITCRAS |
| | (SEQ | SYA | AFDIW | N | ID | (SEQ | FTSYYMHWVRQAPG | QSISSSLNWYQQK |
| | ID | (SEQ | (SEQ | (SEQ | NO: | ID | QGLEWMGIINHSGG | PGKAPKLLIYAAS |
| | NO: | ID | ID | ID | 420) | NO: | STSYAQKFQGRVTM | TLQSGVPSRFSGS |
| | 215) | NO: | NO: | NO: | | 429) | TRDTSTSTVYMELS | GSGTDFTLTISSL |
| | | 426) | 427) | 428) | | | SLRSEDTAVYYCAR | QPEDFATYYCQQS |
| | | | | | | | PYSGWYFAFDIWGQ | YSTPLTFGQGTKV |
| | | | | | | | GTLVTVSS (SEQ | EIK (SEQ ID |
| | | | | | | | ID NO: 430) | NO: 431) |
| 10. | FMFGD | SAISG | CAKDL | RASQG | DASSL | CQQTH | EVQLLESGGGLVQP | DIQMTQSPSSLSA |
| | YAMH | SGGST | VVAGI | ISNNL | ES | SFPST | GGSLRLSCAASGFM | SVGDRVTITCRAS |
| | (SEQ | YYA | WYFDL | N | (SEQ | F | FGDYAMHWVRQAPG | QGISNNLNWYQQK |
| | ID | (SEQ | W | (SEQ | ID | (SEQ | KGLEWVSAISGSGG | PGKAPKLLIYDAS |
| | NO: | ID | (SEQ | ID | NO: | ID | STYYADSVKGRFTI | SLESGVPSRESGS |
| | 102) | NO: | ID | NO: | 106) | NO: | SRDNSKNTLYLQMN | GSGTDFTLTISSL |
| | | 103) | NO: | 105) | | 107) | SLRAEDTAVYYCAK | QPEDFATYYCQQT |
| | | | 104) | | | | DLVVAGIWYFDLWG | HSFPSTFGQGTKL |
| | | | | | | | RGTLVTVSS (SEQ | EIK (SEQ ID |
| | | | | | | | ID NO: 432) | NO: 433) |
| 11. | FTFSD | SVIGE | CAADP | RASQG | AASTL | CQQSY | EVQLLESGGGLVQP | DIQMTQSPSSLSA |
| | YYMN | SGGST | VSRWP | ISSSL | QS | STPWT | GGSLRLSCAASGFT | SVGDRVTITCRAS |
| | (SEQ | YYA | KHGGG | A | (SEQ | F | FSDYYMNWVRQAPG | QGISSSLAWYQQK |
| | ID | (SEQ | DYW | (SEQ | ID | (SEQ | KGLEWVSVIGESGG | PGKAPKLLIYAAS |
| | NO: | ID | (SEQ | ID | NO: | ID | STYYADSVKGRFTI | TLQSGVPSRFSGS |
| | | | ID | NO: | 113) | NO: | SRDNSKNTLYLQMN | GSGTDFTLTISSL |
| | 109) | NO: | NO: | 112) | | 114) | SLRAEDTAVYYCAA | QPEDFATYYCQQS |
| | | 110) | 111) | | | | DPVSRWPKHGGGDY | YSTPWTFGQGTKV |
| | | | | | | | WGQGTLVTVSS | EIK (SEQ ID |
| | | | | | | | (SEQ ID NO: | NO: 435) |
| | | | | | | | 434) | |
| 12. | YTLTT | GWINP | CAKGD | RASDN | AASSL | CQQGY | QVQLVQSGAEVKKP | DIQMTQSPSSLSA |
| | WYMY | NRGAT | LWGAM | IGSWL | QS | STPPT | GASVKVSCKASGYT | SVGDRVTITCRAS |
| | (SEQ | NYA | DVW | A | (SEQ | F | LTTWYMYWVRQAPG | DNIGSWLAWYQQK |
| | ID | (SEQ | (SEQ | (SEQ | ID | (SEQ | QGLEWMGWINPNRG | PGKAPKLLIYAAS |
| | NO: | ID | ID | ID | NO: | ID | ATNYAQKFQGRVTM | SLQSGVPSRESGS |
| | 116) | NO: | NO: | NO: | 65) | NO: | TRDTSTSTVYMELS | GSGTDFTLTISSL |
| | | 117) | 118) | 119) | | 120) | SLRSEDTAVYYCAK | QPEDFATYYCQQG |
| | | | | | | | GDLWGAMDVWGQGT | YSTPPTFGQGTKV |
| | | | | | | | LVTVSS (SEQ ID | EIK (SEQ ID |
| | | | | | | | NO: 436) | NO: 437) |
| 13. | YTFTT | GGFDP | CARHA | RASES | AASTL | CQQSY | QVQLVQSGAEVKKP | DIQMTQSPSSLSA |
| | YYMH | EDGET | VAGAV | ISNWL | QS | SVPFT | GASVKVSCKASGYT | SVGDRVTITCRAS |
| | (SEQ | IYA | GAGYY | A | (SEQ | F | FTTYYMHWVRQAPG | ESISNWLAWYQQK |
| | ID | (SEQ | YYGMD | (SEQ | ID | (SEQ | QGLEWMGGFDPEDG | PGKAPKLLIYAAS |
| | NO: | ID | VW | ID | NO: | ID | ETIYAQKFQGRVTM | TLQSGVPSRFSGS |
| | 122) | NO: | (SEQ | NO: | 113) | NO: | TRDTSTSTVYMELS | GSGTDFTLTISSL |
| | | 123) | ID | 125) | | 126) | SLRSEDTAVYYCAR | QPEDFATYYCQQS |
| | | | NO: | | | | HAVAGAVGAGYYYY | YSVPFTFGPGTKV |
| | | | 124) | | | | GMDVWGQGTMVTVS | DIK (SEQ ID |
| | | | | | | | S (SEQ ID NO: | NO: 439) |
| | | | | | | | 438) | |
| 14. | YTFTN | GGIIP | CAKGQ | QANQD | SKLEA | QQSSE | QVQLVQSGAEVKKP | DIQMTQSPSSLSA |
| | YYMH | IVDGV | FTGNY | ISNYL | (SEQ | IPYS | GSSVKVSCKASGYT | SVGDRVTITCQAN |
| | (SEQ | KYA | YYGMD | N | ID | (SEQ | FTNYYMHWVRQAPG | QDISNYLNWYQQK |
| | ID | (SEQ | YW | (SEQ | NO: | ID | QGLEWMGGIIPIVD | PGKAPKLLIYRAS |
| | NO: | ID | (SEQ | ID | 442) | NO: | GVKYAQKFQGRVTI | KLEAGVPSRFSGS |
| | 148) | NO: | ID | NO: | | 443) | TADESTSTAYMELS | GSGTDFTLTISSL |
| | | 440) | NO: | 151) | | | SLRSEDTAVYYCAK | QPEDFATYYCQQS |
| | | | 441) | | | | GQFTGNYYYGMDYW | SEIPYSFGQGTKV |
| | | | | | | | GQGTLVTVSS | EIK (SEQ ID |
| | | | | | | | (SEQ ID NO: | NO: 445) |
| | | | | | | | 444) | |
| 15. | YTFTG | GWIGP | CARDL | RSSQS | SSSNR | CMQAL | QVQLVQSGAEVKKP | DIVMTQSPLSLPV |
| | YYMH | NSGDT | DHNWY | LLHSN | AP | HIPLT | GASVKVSCKASGYT | TPGEPASISCRSS |
| | (SEQ | NYA | FDLW | GYNYL | (SEQ | F | FTGYYMHWVRQAPG | QSLLHSNGYNYLD |
| | ID | (SEQ | (SEQ | D | ID | (SEQ | QGLEWMGWIGPNSG | WYLQKPGQSPQLL |
| | NO: | ID | ID | (SEQ | NO: | ID | DTNYAQKFQGRVTM | IYSSSNRAPGVPD |
| | 128) | NO: | NO: | ID | 132) | NO: | TRDTSTSTVYMELS | RFSGSGSGTDFTL |
| | | 129) | 130) | NO: | | 133) | SLRSEDTAVYYCAR | KISRVEAEDVGVY |
| | | | | 131) | | | DLDHNWYFDLWGRG | YCMQALHIPLTFG |
| | | | | | | | TLVTVSS (SEQ | GGTKVEIK (SEQ |
| | | | | | | | ID NO: 446) | ID NO: 447) |
| 16. | FTFDD | SYIDA | CAKDQ | QASQD | KASTL | CQQSY | EVQLLESGGGLVQP | DIQMTQSPSSLSA |
| | YAMH | SGTTI | AAAGY | ISNYL | ES | STPIT | GGSLRLSCAASGFT | SVGDRVTITCQAS |
| | (SEQ | YYA | WYFDL | N | (SEQ | F | FDDYAMHWVRQAPG | QDISNYLNWYQQK |
| | ID | (SEQ | W | (SEQ | ID | (SEQ | KGLEWVSYIDASGT | PGKAPKLLIYKAS |
| | NO: | ID | (SEQ | ID | NO: | ID | TIYYADSVKGRFTI | TLESGVPSRFSGS |
| | 135) | NO: | ID | NO: | 139) | NO: | SRDNSKNTLYLQMN | GSGTDFTLTISSL |
| | | 136) | NO: | 138) | | 140) | SLRAEDTAVYYCAK | QPEDFATYYCQQS |
| | | | 137) | | | | DQAAAGYWYFDLWG | YSTPITFGQGTRL |
| | | | | | | | RGTLVTVSS (SEQ | EIK (SEQ ID |
| | | | | | | | ID NO: 448) | NO: 449) |
| 17. | YTFTD | GGIVP | CAKDE | RSSQS | SAYNR | CMQAL | QVQLVQSGAEVKKP | DIVMTQSPLSLPV |
| | YHIH | RSGST | SSGWY | LLHSN | AS | QTPLT | GSSVKVSCKASGYT | TPGEPASISCRSS |
| | (SEQ | TYA | YFDYW | GYNYL | (SEQ | F | FTDYHIHWVRQAPG | QSLLHSNGYNYLD |
| | ID | (SEQ | (SEQ | D | ID | (SEQ | QGLEWMGGIVPRSG | WYLQKPGQSPQLL |
| | NO: | ID | ID | (SEQ | NO: | ID | STTYAQKFQGRVTI | IYSAYNRASGVPD |
| | 142) | NO: | NO: | ID | 145) | NO: | TADESTSTAYMELS | RFSGSGSGTDFTL |
| | | 143) | 144 | NO: | | 146) | SLRSEDTAVYYCAK | KISRVEAEDVGVY |
| | | | | 131) | | | DESSGWYYFDYWGQ | YCMQALQTPLTFG |
| | | | | | | | GTLVTVSS (SEQ | QGTKVEIK (SEQ |
| | | | | | | | ID NO: 450) | ID NO: 451) |
| 18. | YTFTN | GGIIP | CAKGR | QANQD | RASKL | CQQSS | QVQLVQSGAEVKKP | DIQMTQSPSSLSA |
| | YYMH | IVDRV | YTVNY | ISNYL | EA | EIPYS | GSSVKVSCKASGYT | SVGDRVTITCQAN |
| | (SEQ | KYA | YYGMD | N | (SEQ | F | FTNYYMHWVRQAPG | QDISNYLNWYQQK |
| | ID | (SEQ | VW | (SEQ | ID | (SEQ | QGLEWMGGIIPIVD | PGKAPKLLIYRAS |
| | NO: | ID | (SEQ | ID | NO: | ID | RVKYAQKFQGRVTI | KLEAGVPSRESGS |
| | 148) | NO: | ID | NO: | 152) | NO: | TADESTSTAYMELS | GSGTDFTLTISSL |
| | | 149) | NO: | 151) | | 153) | SLRSEDTAVYYCAK | QPEDFATYYCQQS |
| | | | 150) | | | | GRYTVNYYYGMDVW | SEIPYSFGQGTKL |
| | | | | | | | GQGTTVTVSS | EIK (SEQ ID |
| | | | | | | | (SEQ ID NO: | NO: 453) |
| | | | | | | | 452) | |
| 19. | FTFED | SYLNS | CAKDY | RASQS | DASNL | CQQSY | EVQLLESGGGLVQP | DIQMTQSPSSLSA |
| | YAMH | DGGST | CTNGV | ISTYL | ET | TIPIT | GGSLRLSCAASGFT | SVGDRVTITCRAS |
| | (SEQ | SYA | CAFDY | N | (SEQ | F | FEDYAMHWVRQAPG | QSISTYLNWYQQK |
| | ID | (SEQ | W | (SEQ | ID | (SEQ | KGLEWVSYLNSDGG | PGKAPKLLIYDAS |
| | NO: | ID | (SEQ | ID | NO: | ID | STSYADSVKGRFTI | NLETGVPSRFSGS |
| | 155) | NO: | ID | NO: | 159) | NO: | SRDNSKNTLYLQMN | GSGTDFTLTISSL |
| | | 156) | NO: | 158) | | 160) | SLRAEDTAVYYCAK | QPEDFATYYCQQS |
| | | | 157) | | | | DYCTNGVCAFDYWG | YTIPITFGQGTRL |
| | | | | | | | QGTLVTVSS (SEQ | EIK (SEQ ID |
| | | | | | | | ID NO: 454) | NO: 455) |
| 20. | FTFSD | SAISG | CVSDI | RASQS | AASRL | CQQAN | EVQLLESGGGLVQP | DIQMTQSPSSLSA |
| | SAMH | SGSTI | AVAGH | ISTFL | EG | SFPLT | GGSLRLSCAASGFT | SVGDRVTITCRAS |
| | (SEQ | YYA | WYFDL | N | (SEQ | F | FSDSAMHWVRQAPG | QSISTFLNWYQQK |
| | ID | (SEQ | W | (SEQ | ID | (SEQ | KGLEWVSAISGSGS | PGKAPKLLIYAAS |
| | NO: | ID | (SEQ | ID | NO: | ID | TIYYADSVKGRFTI | RLEGGVPSRFSGS |
| | 162) | NO: | ID | NO: | 166) | NO: | SRDNSKNTLYLQMN | GSGTDFTLTISSL |
| | | 163) | NO: | 165) | | 167) | SLRAEDTAVYYCVS | QPEDFATYYCQQA |
| | | | 164) | | | | DIAVAGHWYFDLWG | NSFPLTFGPGTKV |
| | | | | | | | RGTLVTVSS (SEQ | DIK (SEQ ID |
| | | | | | | | ID NO: 456) | NO: 457) |
| 21. | FTFSS | SYISG | CARAN | RASQS | AASSL | CQQSY | EVQLVESGGGLVKP | DIQMTQSPSSLSA |
| | YWMS | DSGYT | SSGWY | ISSYL | QS | STPLT | GGSLRLSCAASGFT | SVGDRVTITCRAS |
| | (SEQ | NYA | DWYFD | N | (SEQ | F | FSSYWMSWVRQAPG | QSISSYLNWYQQK |
| | ID | (SEQ | LW | (SEQ | ID | (SEQ | KGLEWVSYISGDSG | PGKAPKLLIYAAS |
| | NO: | ID | (SEQ | ID | NO: | ID | YTNYAAPVKGRFTI | SLQSGVPSRFSGS |
| | 169) | NO: | ID | NO: | 65) | NO: | SRDDSKNTLYLQMN | GSGTDFTLTISSL |
| | | 170) | NO: | 172) | | 173) | SLKTEDTAVYYCAR | QPEDFATYYCQQS |
| | | | 171) | | | | ANSSGWYDWYFDLW | YSTPLTFGGGTKV |
| | | | | | | | GRGTLVTVSS | EIK (SEQ ID |
| | | | | | | | (SEQ ID NO: | NO: 459) |
| | | | | | | | 458) | |
| 22. | FTFDD | SGISW | CAKDI | QASQD | DASNL | CQQSY | EVQLLESGGGLVQP | DIQMTQSPSSLSA |
| | YAMH | NSGSI | VAAGH | ISNYL | ET | STPLT | GGSLRLSCAASGET | SVGDRVTITCQAS |
| | (SEQ | GYA | YYYGM | N | (SEQ | F | FDDYAMHWVRQAPG | QDISNYLNWYQQK |
| | ID | (SEQ | DVW | (SEQ | ID | (SEQ | KGLEWVSGISWNSG | PGKAPKLLIYDAS |
| | NO: | ID | (SEQ | ID | NO: | ID | SIGYADSVKGRFTI | NLETGVPSRFSGS |
| | 135) | NO: | ID | NO: | 159) | NO: | SRDNSKNTLYLQMN | GSGTDFTLTISSL |
| | | 175) | NO: | 138) | | 173) | SLRAEDTAVYYCAK | QPEDFATYYCQQS |
| | | | 176) | | | | DIVAAGHYYYGMDV | YSTPLTFGGGTKV |
| | | | | | | | WGQGTTVTVSS | EIK (SEQ ID |
| | | | | | | | (SEQ ID NO: | NO: 461) |
| | | | | | | | 460) | |
| 23. | FTFDD | SYIDT | CARDE | QAGQD | DASNL | CQQTY | EVQLLESGGGLVQP | DIQMTQSPSSLSA |
| | YAMH | SSSHL | AAAGY | ISNYL | ET | STPIT | GGSLRLSCAASGFT | SVGDRVTITCQAG |
| | (SEQ | YYA | YGMDV | N | (SEQ | F | FDDYAMHWVRQAPG | QDISNYLNWYQQK |
| | ID | (SEQ | W | (SEQ | ID | (SEQ | KGLEWVSYIDTSSS | PGKAPKLLIYDAS |
| | NO: | ID | (SEQ | ID | NO: | ID | HLYYADSVKGRFTI | NLETGVPSRFSGS |
| | 135) | NO: | ID | NO: | 159) | NO: | SRDNSKNTLYLQMN | GSGTDFTLTISSL |
| | | 178) | NO: | 180) | | 181) | SLRAEDTAVYYCAR | QPEDFATYYCQQT |
| | | | 179) | | | | DEAAAGYYGMDVWG | YSTPITFGQGTKL |
| | | | | | | | QGTTVTVSS (SEQ | EIK (SEQ ID |
| | | | | | | | ID NO: 462) | NO: 463) |
| 24. | FTFSS | SRISS | CARGT | RASQS | SNLQS | QQSYS | EVQLVESGGGLVKP | DIQMTQSPSSLSA |
| | YWMS | DGRIT | SYCTG | IGRNL | (SEQ | IPLT | GGSLRLSCAASGET | SVGDRVTITCRAS |
| | (SEQ | TYA | GVCDI | N | ID | (SEQ | FSSYWMSWVRQAPG | QSIGRNLNWYQQK |
| | ID | (SEQ | DYW | (SEQ | NO: | ID | KGLEWVSRISSDGR | PGKAPKLLIYSAS |
| | NO: | ID | (SEQ | ID | 467) | NO: | ITTYAAPVKGRFTI | NLQSGVPSRFSGS |
| | 169) | NO: | ID | NO: | | 468) | SRDDSKNTLYLQMN | GSGTDFTLTISSL |
| | | 464) | NO: | 466) | | | SLKTEDTAVYYCAR | QPEDFATYYCQQS |
| | | | 465) | | | | GTSYCTGGVCDIDY | YSIPLTFGPGTKV |
| | | | | | | | WGQGTLVTVSS | DIK (SEQ ID |
| | | | | | | | (SEQ ID NO: | NO: 470) |
| | | | | | | | 469) | |
| 25. | FTFSN | STIVG | CARDN | RASQD | AASSL | CQQSY | EVQLLESGGGLVQP | DIQMTQSPSSLSA |
| | AWMS | NGGAT | PLRWQ | ISNYL | QS | SIPPT | GGSLRLSCAASGFT | SVGDRVTITCRAS |
| | (SEQ | YYA | GMDVW | N | (SEQ | F | FSNAWMSWVRQAPG | QDISNYLNWYQQK |
| | ID | (SEQ | (SEQ | (SEQ | ID | (SEQ | KGLEWVSTIVGNGG | PGKAPKLLIYAAS |
| | NO: | ID | ID | ID | NO: | ID | ATYYADSVKGRFTI | SLQSGVPSRFSGS |
| | 183) | NO: | NO: | NO: | 65) | NO: | SRDNSKNTLYLQMN | GSGTDFTLTISSL |
| | | 184) | 185) | 186) | | 187) | SLRAEDTAVYYCAR | QPEDFATYYCQQS |
| | | | | | | | DNPLRWQGMDVWGQ | YSIPPTFGPGTKV |
| | | | | | | | GTLVTVSS (SEQ | DIK (SEQ ID |
| | | | | | | | ID NO: 471) | NO: 472) |
| 26. | FTFSS | SYISS | CARAN | RASQS | SGLQS | QQSYS | EVQLLESGGGLVQP | DIQMTQSPSSLSA |
| | YAMS | SSTYT | SSSWY | ISSYL | (SEQ | TPLT | GGSLRLSCAASGFT | SVGDRVTITCRAS |
| | (SEQ | NYA | DWYFD | N | ID | (SEQ | FSSYAMSWVRQAPG | QSISSYLNWYQQK |
| | ID | (SEQ | LW | (SEQ | NO: | ID | KGLEWVSYISSSST | PGKAPKLLIYAAS |
| | NO: | ID | (SEQ | ID | 474) | NO: | YTNYADSVKGRFTI | GLQSGVPSRFSGS |
| | 473) | NO: | ID | NO: | | 429) | SRDNSKNTLYLQMN | GSGTDFTLTISSL |
| | | 190) | NO: | 172) | | | SLRAEDTAVYYCAR | QPEDFATYYCQQS |
| | | | 191) | | | | ANSSSWYDWYFDLW | YSTPLTFGGGTKV |
| | | | | | | | GQGTLVTVSS | EIK (SEQ ID |
| | | | | | | | (SEQ ID NO: | NO: 476) |
| | | | | | | | 475) | |
| 27. | FTFSS | SYISS | CARAN | RASQS | SGLQS | QQSYS | EVQLLESGGGLVQP | DIQMTQSPSSLSA |
| | YQMS | SSTYT | SSSWY | ISSYL | (SEQ | TPLT | GGSLRLSCAASGFT | SVGDRVTITCRAS |
| | (SEQ | NYA | DWYFD | N | ID | (SEQ | FSSYQMSWVRQAPG | QSISSYLNWYQQK |
| | ID | (SEQ | LW | (SEQ | NO: | ID | KGLEWVSYISSSST | PGKAPKLLIYAAS |
| | NO: | ID | (SEQ | ID | 474) | NO: | YTNYADSVKGRFTI | GLQSGVPSRFSGS |
| | 189) | NO: | ID | NO: | | 429) | SRDNSKNTLYLQMN | GSGTDFTLTISSL |
| | | 190) | NO: | 172) | | | SLRAEDTAVYYCAR | QPEDFATYYCQQS |
| | | | 191) | | | | ANSSSWYDWYFDLW | YSTPLTFGGGTKV |
| | | | | | | | GQGTLVTVSS | EIK (SEQ ID |
| | | | | | | | (SEQ ID NO: | NO: 476) |
| | | | | | | | 477) | |
| 28. | FTFSS | SYISS | CARAN | RASQS | SSLQS | QQSYS | EVQLLESGGGLVQP | DIQMTQSPSSLSA |
| | YAMS | SSTYT | SSSWY | ISSYL | (SEQ | TPLT | GGSLRLSCAASGFT | SVGDRVTITCRAS |
| | (SEQ | NYA | DWYFD | N | ID | (SEQ | FSSYAMSWVRQAPG | QSISSYLNWYQQK |
| | ID | (SEQ | LW | (SEQ | NO: | ID | KGLEWVSYISSSST | PGKAPKLLIYAAS |
| | NO: | ID | (SEQ | ID | 362) | NO: | YTNYADSVKGRFTI | SLQSGVPSRESGS |
| | 473) | NO: | ID | NO: | | 429) | SRDNSKNTLYLQMN | GSGTDFTLTISSL |
| | | 190) | NO: | 172) | | | SLRAEDTAVYYCAR | QPEDFATYYCQQS |
| | | | 191) | | | | ANSSSWYDWYFDLW | YSTPLTFGGGTKV |
| | | | | | | | GQGTLVTVSS | EIK (SEQ ID |
| | | | | | | | (SEQ ID NO: | NO: 459) |
| | | | | | | | 475) | |
| 29. | FTFSS | SYISS | CARAN | RASQS | AASSL | CQQSY | EVQLLESGGGLVQP | DIQMTQSPSSLSA |
| | YQMS | SSTYT | SSSWY | ISSYL | QS | STPLT | GGSLRLSCAASGFT | SVGDRVTITCRAS |
| | (SEQ | NYA | DWYFD | N | (SEQ | F | FSSYQMSWVRQAPG | QSISSYLNWYQQK |
| | ID | (SEQ | LW | (SEQ | ID | (SEQ | KGLEWVSYISSSST | PGKAPKLLIYAAS |
| | NO: | ID | (SEQ | ID | NO: | ID | YTNYADSVKGRFTI | SLQSGVPSRESGS |
| | 189) | NO: | ID | NO: | 65) | NO: | SRDNSKNTLYLQMN | GSGTDFTLTISSL |
| | | 190) | NO: | 172) | | 173) | SLRAEDTAVYYCAR | QPEDFATYYCQQS |
| | | | 191) | | | | ANSSSWYDWYFDLW | YSTPLTFGGGTKV |
| | | | | | | | GQGTLVTVSS | EIK (SEQ ID |
| | | | | | | | (SEQ ID NO: | NO: 459) |
| | | | | | | | 477) | |
| 30. | FTFSS | SGISG | CATSQ | RASQS | AASNL | CQQSY | EVQLLESGGGLVQP | DIQMTQSPSSLSA |
| | YAMH | SGGSA | APVDY | ISSWL | QR | SIPIT | GGSLRLSCAASGFT | SVGDRVTITCRAS |
| | (SEQ | YYA | YYYGM | A | (SEQ | F | FSSYAMHWVRQAPG | QSISSWLAWYQQK |
| | ID | (SEQ | DVW | (SEQ | ID | (SEQ | KGLEWVSGISGSGG | PGKAPKLLIYAAS |
| | NO: | ID | (SEQ | ID | NO: | ID | SAYYADSVKGRFTI | NLQRGVPSRFSGS |
| | 193) | NO: | ID | NO: | 197) | NO: | SRDNSKNTLYLQMN | GSGTDFTLTISSL |
| | | 194) | NO: | 196) | | 198) | SLRAEDTAVYYCAT | QPEDFATYYCQQS |
| | | | 195) | | | | SQAPVDYYYYGMDV | YSIPITFGQGTKV |
| | | | | | | | WGQGTTVTVSS | EIK (SEQ ID |
| | | | | | | | (SEQ ID NO: | NO: 479) |
| | | | | | | | 478) | |
| 31. | FTFSS | SYISG | CARVG | RASQS | AASSL | CQQSY | EVQLVESGGGLVKP | DIQMTQSPSSLSA |
| | YWMS | SSSYT | SSGWY | ISSYL | QS | STPLT | GGSLRLSCAASGFT | SVGDRVTITCRAS |
| | (SEQ | NYA | DWYFD | N | (SEQ | F | FSSYWMSWVRQAPG | QSISSYLNWYQQK |
| | ID | (SEQ | LW | (SEQ | ID | (SEQ | KGLEWVSYISGSSS | PGKAPKLLIYAAS |
| | NO: | ID | (SEQ | ID | NO: | ID | YTNYAAPVKGRFTI | SLQSGVPSRFSGS |
| | 169) | NO: | ID | NO: | 65) | NO: | SRDDSKNTLYLQMN | GSGTDFTLTISSL |
| | | 200) | NO: | 172) | | 173) | SLKTEDTAVYYCAR | QPEDFATYYCQQS |
| | | | 201) | | | | VGSSGWYDWYFDLW | YSTPLTFGQGTKV |
| | | | | | | | GRGTLVTVSS | EIK (SEQ ID |
| | | | | | | | (SEQ ID NO: | NO: 481) |
| | | | | | | | 480) | |
| 32. | YTLTT | GWINP | CAKGD | RASDN | AASSL | CQQGY | QVQLVQSGAEVKKP | DIQMTQSPSSLSA |
| | WYMY | NRGAT | LWGAM | IGSWL | QS | STPPT | GASVKVSCKASGYT | SVGDRVTITCRAS |
| | (SEQ | NYA | DVW | A | (SEQ | F | LTTWYMYWVRQAPG | DNIGSWLAWYQQK |
| | ID | (SEQ | (SEQ | (SEQ | ID | (SEQ | QGLEWMGWINPNRG | PGKAPKLLIYAAS |
| | NO: | ID | ID | ID | NO: | ID | ATNYAQKFQGRVTM | SLQSGVPSRFSGS |
| | 116) | NO: | NO: | NO: | 65) | NO: | TRDTSTSTVYMELS | GSGTDFTLTISSL |
| | | 117) | 118) | 119) | | 120) | SLRSEDTAVYYCAK | QPEDFATYYCQQG |
| | | | | | | | GDLWGAMDVWGQGT | YSTPPTFGQGTKV |
| | | | | | | | LVTVSS (SEQ ID | EIK (SEQ ID |
| | | | | | | | NO: 436) | NO: 437) |
| 33. | YTLTT | GWINP | CAKGD | RASDN | AASSL | CQQGY | QVQLVQSGAEVKKP | DIQMTQSPSSLSA |
| | WYMY | NRGAT | LWGAM | IGSWL | QS | STPPT | GASVKVSCKASGYT | SVGDRVTITCRAS |
| | (SEQ | NYA | DVW | A | (SEQ | F | LTTWYMYWVRQAPG | DNIGSWLAWYQQK |
| | ID | (SEQ | (SEQ | (SEQ | ID | (SEQ | QGLEWMGWINPNRG | PGKAPKLLIYAAS |
| | NO: | ID | ID | ID | NO: | ID | ATNYAQKFQGRVTM | SLQSGVPSRFSGS |
| | 116) | NO: | NO: | NO: | 65) | NO: | TRDTSTSTVYMELS | GSGTDFTLTISSL |
| | | 117) | 118) | 119) | | 120) | SLRSEDTAVYYCAK | QPEDFATYYCQQG |
| | | | | | | | GDLWGAMDVWGQGT | YSTPPTFGQGTKV |
| | | | | | | | TVTVSS (SEQ ID | EIK (SEQ ID |
| | | | | | | | NO: 482) | NO: 437) |
| 34. | YTFTG | GWMNP | CARDP | RASQS | AASSL | CQQSY | QVQLVQSGAEVKKP | DIQMTQSPSSLSA |
| | YYIH | NSGNT | GFLGY | ISSYL | QS | TAPYT | GASVKVSCKASGYT | SVGDRVTITCRAS |
| | (SEQ | GYA | CSGGS | H | (SEQ | F | FTGYYIHWVRQAPG | QSISSYLHWYQQK |
| | ID | (SEQ | CYDGW | (SEQ | ID | (SEQ | QGLEWMGWMNPNSG | PGKAPKLLIYAAS |
| | NO: | ID | FDPW | ID | NO: | ID | NTGYAQKFQGRVTM | SLQSGVPSRESGS |
| | 204) | NO: | (SEQ | NO: | 65) | NO: | TRDTSTSTVYMELS | GSGTDFTLTISSL |
| | | 205) | ID | 207) | | 208) | SLRSEDTAVYYCAR | QPEDFATYYCQQS |
| | | | NO: | | | | DPGFLGYCSGGSCY | YTAPYTFGQGTKL |
| | | | 206) | | | | DGWFDPWGQGTLVT | EIK (SEQ ID |
| | | | | | | | VSS (SEQ ID | NO: 484) |
| | | | | | | | NO: 483) | |
| 35. | YTFTG | GWMNP | CARDP | RASQS | AASSL | CQQSY | QVQLVQSGAEVKKP | DIQMTQSPSSLSA |
| | YYIH | NSGNT | GFLGY | ISSYL | QS | TAPYT | GASVKVSCKASGYT | SVGDRVTITCRAS |
| | (SEQ | GYA | SSGGS | H | (SEQ | F | FTGYYIHWVRQAPG | QSISSYLHWYQQK |
| | ID | (SEQ | CYDGW | (SEQ | ID | (SEQ | QGLEWMGWMNPNSG | PGKAPKLLIYAAS |
| | NO: | ID | FDPW | ID | NO: | ID | NTGYAQKFQGRVTM | SLQSGVPSRFSGS |
| | 204) | NO: | (SEQ | NO: | 65) | NO: | TRDTSTSTVYMELS | GSGTDFTLTISSL |
| | | 205) | ID | 207) | | 208) | SLRSEDTAVYYCAR | QPEDFATYYCQQS |
| | | | NO: | | | | DPGFLGYSSGGSSY | YTAPYTFGQGTKL |
| | | | 485) | | | | DGWFDPWGQGTLVT | EIK (SEQ ID |
| | | | | | | | VSS (SEQ ID | NO: 484) |
| | | | | | | | NO: 486) | |
| 36. | FTFDD | SAISG | CARDG | QASQD | SNLET | QQSYS | EVQLLESGGGLVQP | DIQMTQSPSSLSA |
| | YALH | DGRST | TVNGA | ISKYL | (SEQ | IPFT | GGSLRLSCAASGFT | SVGDRVTITCQAS |
| | (SEQ | TYA | TGWFD | N | ID | (SEQ | FDDYALHWVRQAPG | QDISKYLNWYQQK |
| | ID | (SEQ | PW | (SEQ | NO: | ID | KGLEWVSAISGDGR | PGKAPKLLIYDAS |
| | NO: | ID | (SEQ | ID | 491) | NO: | STTYADSVKGRFTI | NLETGVPSRFSGS |
| | 487) | NO: | ID | NO: | | 492) | SRDNSKNTLYLQMN | GSGTDFTLTISSL |
| | | 488) | NO: | 490) | | | SLRAEDTAVYYCAR | QPEDFATYYCQQS |
| | | | 489) | | | | DGTVNGATGWFDPW | YSIPFTFGPGTKV |
| | | | | | | | GQGTLVTVSS | DIK (SEQ ID |
| | | | | | | | (SEQ ID NO: | NO: 494) |
| | | | | | | | 493) | |
| 37. | FTFSD | SAISG | CARDG | RASQG | SNLET | QQSYS | EVQLLESGGGLVQP | DIQMTQSPSSLSA |
| | YGMP | SGGST | GWQPA | ISNNL | (SEQ | TPLT | GGSLRLSCAASGFT | SVGDRVTITCRAS |
| | (SEQ | YYA | AILDY | N | ID | (SEQ | FSDYGMPWVRQAPG | QGISNNLNWYQQK |
| | ID | (SEQ | W | (SEQ | NO: | ID | KGLEWVSAISGSGG | PGKAPKLLIYDAS |
| | NO: | ID | (SEQ | ID | 491) | NO: | STYYADSVKGRFTI | NLETGVPSRESGS |
| | 495) | NO: | ID | NO: | | 429) | SRDNSKNTLYLQMN | GSGTDFTLTISSL |
| | | 103) | NO: | 105) | | | SLRAEDTAVYYCAR | QPEDFATYYCQQS |
| | | | 296 | | | | DGGWQPAAILDYWG | YSTPLTFGQGTKV |
| | | | | | | | QGTLVTVSS (SEQ | EIK (SEQ ID |
| | | | | | | | ID NO: 496) | NO: 497) |
| 38. | YTFTD | GWMNP | CAREG | RASQG | DASNL | CQQSY | QVQLVQSGAEVKKP | DIQMTQSPSSLSA |
| | YFLH | TSGNT | EGSGF | INSWL | ET | STPLT | GASVKVSCKASGYT | SVGDRVTITCRAS |
| | (SEQ | GYA | DYW | A | (SEQ | F | FTDYFLHWVRQAPG | QGINSWLAWYQQK |
| | ID | (SEQ | (SEQ | (SEQ | ID | (SEQ | QGLEWMGWMNPTSG | PGKAPKLLIYDAS |
| | NO: | ID | ID | ID | NO: | ID | NTGYAQKFQGRVTM | NLETGVPSRFSGS |
| | 210) | NO: | NO: | NO: | 159) | NO: | TRDTSTSTVYMELS | GSGTDFTLTISSL |
| | | 211) | 212 | 213) | | 173) | SLRSEDTAVYYCAR | QPEDFATYYCQQS |
| | | | | | | | EGEGSGFDYWGQGT | YSTPLTFGGGTKV |
| | | | | | | | LVTVSS (SEQ ID | EIK (SEQ ID |
| | | | | | | | NO: 498) | NO: 499) |
| 39. | YTFTS | AWMNP | CARDY | RASQG | AASSL | CQQSY | QVQLVQSGAEVKKP | DIQMTQSPSSLSA |
| | YYMH | NSGNT | DFWSG | ISNYL | QS | STPWT | GASVKVSCKASGYT | SVGDRVTITCRAS |
| | (SEQ | GYA | SLGYW | A | (SEQ | F | FTSYYMHWVRQAPG | QGISNYLAWYQQK |
| | ID | (SEQ | (SEQ | (SEQ | ID | (SEQ | QGLEWMAWMNPNSG | PGKAPKLLIYAAS |
| | NO: | ID | ID | ID | NO: | ID | NTGYAQKFQGRVTM | SLQSGVPSRESGS |
| | 215) | NO: | NO: | NO: | 65) | NO: | TRDTSTSTVYMELS | GSGTDFTLTISSL |
| | | 216) | 217) | 218) | | 114) | SLRSEDTAVYYCAR | QPEDFATYYCQQS |
| | | | | | | | DYDFWSGSLGYWGQ | YSTPWTFGQGTKV |
| | | | | | | | GTLVTVSS (SEQ | EIK (SEQ ID |
| | | | | | | | ID NO: 500) | NO: 501) |
| 40. | YTLTT | GWINP | CAKGD | RASDN | AASSL | CQQGY | QVQLVQSGAEVKKP | DIQMTQSPSSLSA |
| | WYMY | NRGAT | LWGAM | IGSWL | QS | STPPT | GASVKVSCKASGYT | SVGDRVTITCRAS |
| | (SEQ | NYA | DVW | A | (SEQ | F | LTTWYMYWVRQAPG | DNIGSWLAWYQQK |
| | ID | (SEQ | (SEQ | (SEQ | ID | (SEQ | QGLEWMGWINPNRG | PGKAPKLLIYAAS |
| | NO: | ID | ID | ID | NO: | ID | ATNYAQKFQGRVTM | SLQSGVPSRFSGS |
| | 116) | NO: | NO: | NO: | 65) | NO: | TRDTSTSTVYMELS | GSGTDFTLTISSL |
| | | 117) | 118) | 119) | | 120) | SLRSEDTAVYYCAK | QPEDFATYYCQQG |
| | | | | | | | GDLWGAMDVWGQGT | YSTPPTFGQGTKV |
| | | | | | | | LVTVSS (SEQ ID | EIK (SEQ ID |
| | | | | | | | NO: 436) | NO: 437) |
| 41. | YTFTS | GIINP | CARDT | RASQS | DASNL | CQQSY | QVQLVQSGAEVKKP | DIQMTQSPSSLSA |
| | YYMH | SGGST | GYSYG | IGRWL | QS | SIPIT | GASVKVSCKASGYT | SVGDRVTITCRAS |
| | (SEQ | SYA | RYYYY | A | (SEQ | F | FTSYYMHWVRQAPG | QSIGRWLAWYQQK |
| | ID | (SEQ | GMDVW | (SEQ | ID | (SEQ | QGLEWMGIINPSGG | PGKAPKLLIYDAS |
| | NO: | ID | (SEQ | ID | NO: | ID | STSYAQKFQGRVTM | NLQSGVPSRFSGS |
| | 215) | NO: | ID | NO: | 222) | NO: | TRDTSTSTVYMELS | GSGTDFTLTISSL |
| | | 69) | NO: | 221) | | 198) | SLRSEDTAVYYCAR | QPEDFATYYCQQS |
| | | | 220) | | | | DTGYSYGRYYYYGM | YSIPITFGQGTKV |
| | | | | | | | DVWGQGTLVTVSS | EIK (SEQ ID |
| | | | | | | | (SEQ ID NO: | NO: 503) |
| | | | | | | | 502) | |
| 42. | YTLTD | GIINP | CAREE | RASQG | AASSL | CQQSY | QVQLVQSGAEVKKP | DIQMTQSPSSLSA |
| | YYMH | SGGST | YSSSS | ISSWL | QS | STPLT | GASVKVSCKASGYT | SVGDRVTITCRAS |
| | (SEQ | SYA | GYFDY | A | (SEQ | F | LTDYYMHWVRQAPG | QGISSWLAWYQQK |
| | ID | (SEQ | W | (SEQ | ID | (SEQ | QGLEWMGIINPSGG | PGKAPKLLIYAAS |
| | NO: | ID | (SEQ | ID | NO: | ID | STSYAQKFQGRVTM | SLQSGVPSRFSGS |
| | 224) | NO: | ID | NO: | 65) | NO: | TRDTSTSTVYMELS | GSGTDFTLTISSL |
| | | 69) | NO: | 226) | | 173) | SLRSEDTAVYYCAR | QPEDFATYYCQQS |
| | | | 225) | | | | EEYSSSSGYFDYWG | YSTPLTFGQGTKV |
| | | | | | | | QGTLVTVSS (SEQ | EIK (SEQ ID |
| | | | | | | | ID NO: 504) | NO: 505) |
| 43. | YTFTS | GWMHP | CARDT | RASQS | AASSL | CQQSY | QVQLVQSGAEVKKP | DIQMTQSPSSLSA |
| | YGIS | KSGDT | PYYYY | ISSWL | QS | SVPIT | GASVKVSCKASGYT | SVGDRVTITCRAS |
| | (SEQ | GLT | GMDVW | A | (SEQ | F | FTSYGISWVRQAPG | QSISSWLAWYQQK |
| | ID | (SEQ | (SEQ | (SEQ | ID | (SEQ | QGLEWMGWMHPKSG | PGKAPKLLIYAAS |
| | NO: | ID | ID | ID | NO: | ID | DTGLTQKFQGRVTM | SLQSGVPSRFSGS |
| | 228) | NO: | NO: | NO: | 65) | NO: | TRDTSTSTVYMELS | GSGTDFTLTISSL |
| | | 229) | 230) | 196) | | 231) | SLRSEDTAVYYCAR | QPEDFATYYCQQS |
| | | | | | | | DTPYYYYGMDVWGQ | YSVPITFGQGTKV |
| | | | | | | | GTTVTVSS (SEQ | EIK (SEQ ID |
| | | | | | | | ID NO: 506) | NO: 507) |
| 44. | FTFSS | SAISG | CAKER | QASQD | SSLQS | QQTYS | EVQLLESGGGLVQP | DIQMTQSPSSLSA |
| | YAMS | SGGST | FIDYG | ISNYL | (SEQ | GWT | GGSLRLSCAASGFT | SVGDRVTITCQAS |
| | (SEQ | YYA | MDVW | N | ID | (SEQ | FSSYAMSWVRQAPG | QDISNYLNWYQQK |
| | ID | (SEQ | (SEQ | (SEQ | NO: | ID | KGLEWVSAISGSGG | PGKAPKLLIYAAS |
| | NO: | ID | ID | ID | 362) | NO: | STYYADSVKGRFTI | SLQSGVPSRFSGS |
| | 473) | NO: | NO: | NO: | | 509 | SRDNSKNTLYLQMN | GSGTDFTLTISSL |
| | | 103) | 508) | 138) | | | SLRAEDTAVYYCAK | QPEDFATYYCQQT |
| | | | | | | | ERFIDYGMDVWGQG | YSGWTFGPGTKVD |
| | | | | | | | TTVTVSS (SEQ | IK (SEQ ID |
| | | | | | | | ID NO: 510) | NO: 511) |
| 45. | FTFGD | SYISG | CARDV | RASQS | AASSL | CQQSY | EVQLVESGGGLVKP | DIQMTQSPSSLSA |
| | YAMS | DIGYT | AATGN | ISSYL | QS | STPLT | GGSLRLSCAASGFT | SVGDRVTITCRAS |
| | (SEQ | NYA | WYFDL | N | (SEQ | F | FGDYAMSWVRQAPG | QSISSYLNWYQQK |
| | ID | (SEQ | W | (SEQ | ID | (SEQ | KGLEWVSYISGDIG | PGKAPKLLIYAAS |
| | NO: | ID | (SEQ | ID | NO: | ID | YTNYAAPVKGRFTI | SLQSGVPSRFSGS |
| | 233) | NO: | ID | NO: | 65) | NO: | SRDDSKNTLYLQMN | GSGTDFTLTISSL |
| | | 234) | NO: | 172) | | 173) | SLKTEDTAVYYCAR | QPEDFATYYCQQS |
| | | | 235) | | | | DVAATGNWYFDLWG | YSTPLTFGGGTKV |
| | | | | | | | RGTLVTVSS (SEQ | EIK (SEQ ID |
| | | | | | | | ID NO: 512) | NO: 459) |
| 46. | FSFSS | SFITS | CARDR | RASQS | GASTR | CQQYG | EVQLLESGGGLVQP | EIVMTQSPATLSV |
| | YTMN | SSRTI | RGDYG | VRNYL | AT | SSPLT | GGSLRLSCAASGFS | SPGERATLSCRAS |
| | (SEQ | YYA | DSWYF | A | (SEQ | F | FSSYTMNWVRQAPG | QSVRNYLAWYQQK |
| | ID | (SEQ | DLW | (SEQ | ID | (SEQ | KGLEWVSFITSSSR | PGQAPRLLIYGAS |
| | NO: | ID | (SEQ | ID | NO: | ID | TIYYADSVKGRFTI | TRATGIPARFSGS |
| | 237) | NO: | ID | NO: | 241) | NO: | SRDNSKNTLYLQMN | GSGTEFTLTISSL |
| | | 238) | NO: | 240) | | 242) | SLRAEDTAVYYCAR | QSEDFAVYYCQQY |
| | | | 239) | | | | DRRGDYGDSWYFDL | GSSPLTFGGGTKV |
| | | | | | | | WGRGTLVTVSS | EIK (SEQ ID |
| | | | | | | | (SEQ ID NO: | NO: 514) |
| | | | | | | | 513) | |
| 47. | YTFTG | GIINP | CARDT | RASQS | DASNL | CQQSY | QVQLVQSGAEVKKP | DIQMTQSPSSLSA |
| | HYMH | SGGST | GYSYG | IGRWL | QS | SIPIT | GASVKVSCKASGYT | SVGDRVTITCRAS |
| | (SEQ | SYA | RYYYY | A | (SEQ | F | FSKHFVHWVRQAPG | QSISSWLAWYQQK |
| | ID | (SEQ | GMDVW | (SEQ | ID | (SEQ | QGLEWMGWMNPNSG | PGKAPKLLIYAAS |
| | NO: | ID | (SEQ | ID | NO: | ID | NSGYAQKFQGRVTM | TLQSGVPSRFSGS |
| | 244) | NO: | ID | NO: | 222) | NO: | TRDTSTSTVYMELS | GSGTDFTLTISSL |
| | | 69) | NO: | 221) | | 198) | SLRSEDTAVYYCAR | QPEDFATYYCQQS |
| | | | 220) | | | | GEGGYYYYGMDVWG | YSTPWTFGQGTKV |
| | | | | | | | QGTLVTVSS (SEQ | EIK (SEQ ID |
| | | | | | | | ID NO: 515) | NO: 516) |
| 48. | YTFSK | GWMNP | CARGE | RASQS | AASTL | CQQSY | EVQLLESGGGLVQP | DIQMTQSPSSLSA |
| | HFVH | NSGNS | GGYYY | ISSWL | QS | STPWT | GGSLRLSCAASGFT | SVGDRVTITCRAS |
| | (SEQ | GYA | YGMDV | A | (SEQ | F | FGSYSMSWVRQAPG | QPLSNWLAWYQQK |
| | ID | (SEQ | W | (SEQ | ID | (SEQ | KGLEWVSAIGTGGG | PGKAPKLLIYAAS |
| | NO: | ID | (SEQ | ID | NO: | ID | TYYADSVKGRFTIS | SLQSGVPSRFSGS |
| | 246) | NO: | ID | NO: | 113) | NO: | RDNSKNTLYLQMNS | GSGTDFTLTISSL |
| | | 247) | NO: | 196) | | 114) | LRAEDTAVYYCAKG | QPEDFATYYCQQA |
| | | | 248) | | | | TPYYYYYGMDVWGQ | ISFPLTFGGGTKV |
| | | | | | | | GTMVTVSS (SEQ | EIK (SEQ ID |
| | | | | | | | ID NO: 517) | NO: 518) |
| 49. | FTFGS | SAIGT | CAKGT | RASQP | AASSL | CQQAI | QVQLVQSGAEVKKP | DIQMTQSPSSLSA |
| | YSMS | GGGTY | PYYYY | LSNWL | QS | SFPLT | GASVKVSCKASGYT | SVGDRVTITCQSS |
| | SEQ | YA | YGMDV | A | (SEQ | F | FTSYYMHWVRQAPG | EDISSSLNWYQQK |
| | ID | (SEQ | W | (SEQ | ID | (SEQ | QGLEWMGWMNPNSG | PGKAPKLLIYAAS |
| | NO: | ID | SEQ | ID | NO: | ID | NTGYAQKFQGRVTM | SLQIGVPSRFSGS |
| | 250) | NO: | ID | NO: | 65) | NO: | TRDTSTSTVYMELS | GSGTDFTLTISSL |
| | | 251) | NO: | 253) | | 254) | SLRSEDTAVYYCAR | QPEDFATYYCQQT |
| | | | 252) | | | | DLGYYDSSGYFGAF | YSTPYTFGQGTKV |
| | | | | | | | DIWGQGTTVTVSS | EIK (SEQ ID |
| | | | | | | | (SEQ ID NO: | NO: 520) |
| | | | | | | | 519) | |
| 50. | YTFTS | GWMNP | CARDL | QSSED | AASSL | CQQTY | QVQLVQSGAEVKKP | DIQMTQSPSSLSA |
| | YYMH | NSGNT | GYYDS | ISSSL | QI | STPYT | GASVKVSCKASGYT | SVGDRVTITCRAS |
| | (SEQ | GYA | SGYFG | N | (SEQ | F | FTSYGISWVRQAPG | QGIGNWLAWYQQK |
| | ID | (SEQ | AFDIW | (SEQ | ID | (SEQ | QGLEWMGIINPRGG | PGKAPKLLIYAAS |
| | NO: | ID | (SEQ | ID | NO: | ID | STIFAQKFQGRVTM | NLETGVPSRFSGS |
| | 215) | NO: | ID | NO: | 258) | NO: | TRDTSTSTVYMELS | GSGTDFTLTISSL |
| | | 205) | NO: | 257) | | 259) | SLRSEDTAVYYCAR | QPEDFATYYCQQI |
| | | | 256) | | | | GTRSSGWYGWFDPW | HSYPLTFGGGTKV |
| | | | | | | | GQGTLVTVSS | EIK (SEQ ID |
| | | | | | | | (SEQ ID NO: | NO: 522 |
| | | | | | | | 521) | |
| 51. | YTFTS | GIINP | CARGT | RASQG | AASNL | CQQIH | EVQLVESGGGLVKP | DIVMTQSPLSLPV |
| | YGIS | RGGST | RSSGW | IGNWL | ET | SYPLT | GGSLRLSCAASGFI | TPGEPASISCRSS |
| | (SEQ | IFA | YGWFD | A | (SEQ | F | FQDSAIHWVRQAPG | QSLLHSNGYNYLD |
| | ID | (SEQ | PW | (SEQ | ID | (SEQ | KGLEWVSAIGTGGG | WYLQKPGQSPQLL |
| | NO: | ID | (SEQ | ID | NO: | ID | TYYAAPVKGRFTIS | IYDASNLETGVPD |
| | 228) | NO: | ID | NO: | 264) | NO: | RDDSKNTLYLQMNS | RFSGSGSGTDFTL |
| | | 261) | NO: | 263) | | 265) | LKTEDTAVYYCARS | KISRVEAEDVGVY |
| | | | 262) | | | | YCSGGSCSLGSWGQ | YCMQALQTPLTFG |
| | | | | | | | GTLVTVSS (SEQ | QGTKVEIK (SEQ |
| | | | | | | | ID NO: 523) | ID NO: 524) |
| 52. | FTFDD | SYISS | CAREI | RASQS | AASSL | CQQSY | EVQLLESGGGLVQP | DIQMTQSPSSLSA |
| | YGMS | SSSYI | AAAGF | ISSYL | QS | STPLT | GGSLRLSCAASGFT | SVGDRVTITCRAS |
| | (SEQ | YYA | YGMDV | N | (SEQ | F | FDDYGMSWVRQAPG | QSISSYLNWYQQK |
| | ID | (SEQ | W | (SEQ | ID | (SEQ | KGLEWVSYISSSSS | PGKAPKLLIYAAS |
| | NO: | ID | (SEQ | ID | NO: | ID | YIYYADSVKGRFTI | SLQSGVPSRFSGS |
| | 267) | NO: | ID | NO: | 65) | NO: | SRDNSKNTLYLQMN | GSGTDFTLTISSL |
| | | 268) | NO: | 172) | | 173) | SLRAEDTAVYYCAR | QPEDFATYYCQQS |
| | | | 269) | | | | EIAAAGFYGMDVWG | YSTPLTFGGGTKV |
| | | | | | | | QGTTVTVSS (SEQ | EIK (SEQ ID |
| | | | | | | | ID NO: 525) | NO: 459 |
| 53. | YTFTS | GWMNP | CAREG | RASQG | SSLQS | QQSYS | QVQLVQSGAEVKKP | DIQMTQSPSSLSA |
| | YYMH | NSGNT | LGYCT | ISSWL | (SEQ | TPYT | GASVKVSCKASGYT | SVGDRVTITCRAS |
| | (SEQ | GYA | NGVCW | A | ID | (SEQ | FTSYYMHWVRQAPG | QGISSWLAWYQQK |
| | ID | (SEQ | NYYGM | (SEQ | NO: | ID | QGLEWMGWMNPNSG | PGKAPKLLIYGAS |
| | NO: | ID | DVW | ID | 362) | NO: | NTGYAQKFQGRVTM | SLQSGVPSRFSGS |
| | 215) | NO: | (SEQ | NO: | | 527) | TRDTSTSTVYMELS | GSGTDFTLTISSL |
| | | 205) | ID | 226) | | | SLRSEDTAVYYCAR | QPEDFATYYCQQS |
| | | | NO: | | | | EGLGYCTNGVCWNY | YSTPYTFGQGTKV |
| | | | 526) | | | | YGMDVWGQGTLVTV | EIK (SEQ ID |
| | | | | | | | SS (SEQ ID NO: | NO: 529 |
| | | | | | | | 528) | |
| 54. | GTLSR | GGIIP | CARDR | RASQS | GASTR | CQQYG | QVQLVQSGAEVKKP | EIVMTQSPATLSV |
| | YGVS | IFGTT | VYYDS | VSSSY | AT | SSPIT | GSSVKVSCKASGGT | SPGERATLSCRAS |
| | (SEQ | NYA | SGYPT | LA | (SEQ | F | LSRYGVSWVRQAPG | QSVSSSYLAWYQQ |
| | ID | (SEQ | WYFDL | (SEQ | ID | (SEQ | QGLEWMGGIIPIFG | KPGQAPRLLIYGA |
| | NO: | ID | W | ID | NO: | ID | TTNYAQKFQGRVTI | STRATGIPARFSG |
| | 271) | NO: | (SEQ | NO: | 241) | NO: | TADESTSTAYMELS | SGSGTEFTLTISS |
| | | 272) | ID | 274) | | 275) | SLRSEDTAVYYCAR | LQSEDFAVYYCQQ |
| | | | NO: | | | | DRVYYDSSGYPTWY | YGSSPITFGQGTK |
| | | | 273) | | | | FDLWGRGTLVTVSS | VEIK (SEQ ID |
| | | | | | | | (SEQ ID NO: | NO: 531) |
| | | | | | | | 530) | |
| 55. | FTFDD | SGISG | CARDA | QASQD | KASTL | CQQAN | EVQLLESGGGLVQP | DIQMTQSPSSLSA |
| | FAMH | NGDSR | SYGGN | IRNYL | ES | SFPLT | GGSLRLSCAASGFT | SVGDRVTITCQAS |
| | (SEQ | YYA | YGMDV | N | (SEQ | F | FDDFAMHWVRQAPG | QDIRNYLNWYQQK |
| | ID | (SEQ | W | (SEQ | ID | (SEQ | KGLEWVSGISGNGD | PGKAPKLLIYKAS |
| | NO: | ID | (SEQ | ID | NO: | ID | SRYYADSVKGRFTI | TLESGVPSRFSGS |
| | 277) | NO: | ID | NO: | 139) | NO: | SRDNSKNTLYLQMN | GSGTDFTLTISSL |
| | | 278) | NO: | 280) | | 167) | SLRAEDTAVYYCAR | QPEDFATYYCQQA |
| | | | 279) | | | | DASYGGNYGMDVWG | NSFPLTFGPGTKV |
| | | | | | | | QGTTVTVSS (SEQ | DIK (SEQ ID |
| | | | | | | | ID NO: 532) | NO: 533) |
| 56. | FTFSS | SAIGT | CAREW | RASQS | GASNL | CQQSY | EVQLVESGGGLVKP | DIQMTQSPSSLSA |
| | YWMS | GGGTY | LVPYY | ISRWL | QS | STPWT | GGSLRLSCAASGFT | SVGDRVTITCRAS |
| | (SEQ | YA | GMDVW | A | (SEQ | F | FSSYWMSWVRQAPG | QSISRWLAWYQQK |
| | ID | (SEQ | (SEQ | (SEQ | ID | (SEQ | KGLEWVSAIGTGGG | PGKAPKLLIYGAS |
| | NO: | ID | ID | ID | NO: | ID | TYYAAPVKGRFTIS | NLQSGVPSRFSGS |
| | 169) | NO: | NO: | NO: | 284) | NO: | RDDSKNTLYLQMNS | GSGTDFTLTISSL |
| | | 251) | 282) | 283) | | 114) | LKTEDTAVYYCARE | QPEDFATYYCQQS |
| | | | | | | | WLVPYYGMDVWGQG | YSTPWTFGQGTKV |
| | | | | | | | TTVTVSS (SEQ | EIK (SEQ ID |
| | | | | | | | ID NO: 534) | NO: 535) |
| 57. | FSVSS | AGISY | CARSR | KSSQS | WASTR | CHQYY | EVQLLESGGGLVQP | DIVMTQSPDSLAV |
| | NYMS | DGSSK | GIAAR | VLYSS | QS | GHPPT | GGSLRLSCAASGES | SLGERATINCKSS |
| | (SEQ | PYA | PLQHW | NNKNY | (SEQ | F | VSSNYMSWVRQAPG | QSVLYSSNNKNYL |
| | ID | (SEQ | (SEQ | LA | ID | (SEQ | KGLEWVAGISYDGS | AWYQQKPGQPPKL |
| | NO: | ID | ID | (SEQ | NO: | ID | SKPYADSVKGRFTI | LIYWASTRQSGVP |
| | 286) | NO: | NO: | ID | 290) | NO: | SRDNSKNTLYLQMN | DRFSGSGSGTDFT |
| | | 287) | 288) | NO: | | 291) | SLRAEDTAVYYCAR | LTISSLQAEDVAV |
| | | | | 289) | | | SRGIAARPLQHWGQ | YYCHQYYGHPPTF |
| | | | | | | | GTLVTVSS (SEQ | GGGTKVEIK |
| | | | | | | | ID NO: 536) | (SEQ ID NO: |
| | | | | | | | | 537) |
| 58. | FSVSS | AGISY | CARSR | KSSQS | QASTR | CHQYY | EVQLLESGGGLVQP | DIVMTQSPDSLAV |
| | NYMS | DGSSK | GIAAR | VLYSS | QS | GHPPT | GGSLRLSCAASGES | SLGERATINCKSS |
| | (SEQ | PYA | PLQHW | NNKNY | (SEQ | F | VSSNYMSWVRQAPG | QSVLYSSNNKNYL |
| | ID | (SEQ | (SEQ | LA | ID | (SEQ | KGLEWVAGISYDGS | AWYQQKPGQPPKL |
| | NO: | ID | ID | (SEQ | NO: | ID | SKPYADSVKGRFTI | LIYQASTRQSGVP |
| | 286) | NO: | NO: | ID | 293) | NO: | SRDNSKNTLYLQMN | DRFSGSGSGTDFT |
| | | 287) | 288) | NO: | | 291) | SLRAEDTAVYYCAR | LTISSLQAEDVAV |
| | | | | 289) | | | SRGIAARPLQHWGQ | YYCHQYYGHPPTF |
| | | | | | | | GTLVTVSS (SEQ | GGGTKVEIK |
| | | | | | | | ID NO: 536) | (SEQ ID NO: |
| | | | | | | | | 538) |
| 59. | FSFSD | SAISG | CARDG | RASQG | DASNL | CQQSY | EVQLLESGGGLVQP | DIQMTQSPSSLSA |
| | YGMH | SGGST | GWQPA | ISNNL | ET | STPLT | GGSLRLSCAASGFS | SVGDRVTITCRAS |
| | (SEQ | YYA | AILDY | N | (SEQ | F | FSDYGMHWVRQAPG | QGISNNLNWYQQK |
| | ID | (SEQ | W | (SEQ | ID | (SEQ | KGLEWVSAISGSGG | PGKAPKLLIYDAS |
| | NO: | ID | (SEQ | ID | NO: | ID | STYYADSVKGRFTI | NLETGVPSRFSGS |
| | 295) | NO: | ID | NO: | 159) | NO: | SRDNSKNTLYLQMN | GSGTDFTLTISSL |
| | | 103) | NO: | 105) | | 173) | SLRAEDTAVYYCAR | QPEDFATYYCQQS |
| | | | 296) | | | | DGGWQPAAILDYWG | YSTPLTFGGGTKV |
| | | | | | | | QGTLVTVSS (SEQ | EIK (SEQ ID |
| | | | | | | | ID NO: 539) | NO: 540) |
| 60. | FTFSD | SVIYG | CARDP | RASQG | DASNL | CQQSY | EVQLLESGGGLVQP | DIQMTQSPSSLSA |
| | HGMH | GESTY | AVAGG | ISNYL | ET | STCYT | GGSLRLSCAASGET | SVGDRVTITCRAS |
| | (SEQ | YA | GIFDY | A | (SEQ | F | FSDHGMHWVRQAPG | QGISNYLAWYQQK |
| | ID | (SEQ | W | (SEQ | ID | (SEQ | KGLEWVSVIYGGES | PGKAPKLLIYDAS |
| | NO: | ID | (SEQ | ID | NO: | ID | TYYADSVKGRFTIS | NLETGVPSRESGS |
| | 298) | NO: | ID | NO: | 159) | NO: | RDNSKNTLYLQMNS | GSGTDFTLTISSL |
| | | 299) | NO: | 218) | | 301) | LRAEDTAVYYCARD | QPEDFATYYCQQS |
| | | | 300) | | | | PAVAGGGIFDYWGQ | YSTCYTFGQGTKL |
| | | | | | | | GTLVTVSS (SEQ | EIK (SEQ ID |
| | | | | | | | ID NO: 541) | NO: 542) |
| 61. | DTFTG | GWINP | CARSG | RASQT | DASTL | CQQYS | QVQLVQSGAEVKKP | DIQMTQSPSSLSA |
| | YYIH | NSGGT | LWLGS | ISIWL | QS | SYPLT | GASVKVSCKASGDT | SVGDRVTITCRAS |
| | (SEQ | NYA | YYGMD | A | (SEQ | F | FTGYYIHWVRQAPG | QTISIWLAWYQQK |
| | ID | (SEQ | VW | (SEQ | ID | (SEQ | QGLEWMGWINPNSG | PGKAPKLLIYDAS |
| | NO: | ID | (SEQ | ID | NO: | ID | GTNYAQKFQGRVTM | TLQSGVPSRESGS |
| | 303) | NO: | ID | NO: | 307) | NO: | TRDTSTSTVYMELS | GSGTDFTLTISSL |
| | | 304) | NO: | 306) | | 308) | SLRSEDTAVYYCAR | QPEDFATYYCQQY |
| | | | 305) | | | | SGLWLGSYYGMDVW | SSYPLTFGQGTKV |
| | | | | | | | GQGTLVTVSS | EIK (SEQ ID |
| | | | | | | | (SEQ ID NO: | NO: 544) |
| | | | | | | | 543) | |
| 62. | YTFTS | GWINP | CARSP | RASHF | AASTL | CQQSY | QVQLVQSGAEVKKP | DIQMTQSPSSLSA |
| | YDIN | NSGT | YYYYG | ISRWV | QS | SGISF | GASVKVSCKASGYT | SVGDRVTITCRAS |
| | (SEQ | GYA | MDVW | A | (SEQ | (SEQ | FTSYDINWVRQAPG | HFISRWVAWYQQK |
| | ID | (SEQ | (SEQ | (SEQ | ID | ID | QGLEWMGWINPNSG | PGKAPKLLIYAAS |
| | NO: | ID | ID | ID | NO: | NO: | TTGYAQKFQGRVTM | TLQSGVPSRFSGS |
| | 310) | NO: | NO: | NO: | 113) | 314) | TRDTSTSTVYMELS | GSGTDFTLTISSL |
| | | 311) | 312) | 313) | | | SLRSEDTAVYYCAR | QPEDFATYYCQQS |
| | | | | | | | SPYYYYGMDVWGQG | YSGISFGPGTKVD |
| | | | | | | | TTVTVSS (SEQ | IK (SEQ ID |
| | | | | | | | ID NO: 545) | NO: 546) |
| 63. | FTFNN | SRINS | CARGA | RASQS | ATSSR | CQQYY | EVQLLESGGGLVQP | EIVMTQSPATLSV |
| | YGMN | DGSST | YYYYY | VSGSY | AS | SGLTF | GGSLRLSCAASGFT | SPGERATLSCRAS |
| | (SEQ | SYA | MDVW | LA | (SEQ | (SEQ | FNNYGMNWVRQAPG | QSVSGSYLAWYQQ |
| | ID | (SEQ | (SEQ | (SEQ | ID | ID | KGLEWVSRINSDGS | KPGQAPRLLIYAT |
| | NO: | ID | ID | ID | NO: | NO: | STSYADSVKGRFTI | SSRASGIPARFSG |
| | 316) | NO: | NO: | NO: | 320) | 321) | SRDNSKNTLYLQMN | SGSGTEFTLTISS |
| | | 317) | 318) | 319) | | | SLRAEDTAVYYCAR | LQSEDFAVYYCQQ |
| | | | | | | | GAYYYYYMDVWGQG | YYSGLTFGQGTKV |
| | | | | | | | TLVTVSS (SEQ | EIK (SEQ ID |
| | | | | | | | ID NO: 547) | NO: 548) |
| 64. | FTFSN | AHIWN | CARDR | RASQD | DASSL | CQQAT | EVQLLESGGGLVQP | DIQMTQSPSSLSA |
| | SDMN | DGSQK | TDPGY | IRNYL | ET | SLPLT | GGSLRLSCAASGFT | SVGDRVTITCRAS |
| | (SEQ | YYA | SSAMD | G | (SEQ | F | FSNSDMNWVRQAPG | QDIRNYLGWYQQK |
| | ID | (SEQ | VW | (SEQ | ID | (SEQ | KGLEWVAHIWNDGS | PGKAPKLLIYDAS |
| | NO: | ID | (SEQ | ID | NO: | ID | QKYYADSVKGRFTI | SLETGVPSRFSGS |
| | 323) | NO: | ID | NO: | 327) | NO: | SRDNSKNTLYLQMN | GSGTDFTLTISSL |
| | | 324) | NO: | 326 | | 328) | SLRAEDTAVYYCAR | QPEDFATYYCQQA |
| | | | 325) | | | | DRTDPGYSSAMDVW | TSLPLTFGGGTKV |
| | | | | | | | GQGTTVTVSS | EIK (SEQ ID |
| | | | | | | | (SEQ ID NO: | NO: 550) |
| | | | | | | | 549) | |
| 65. | YTFTS | GWMNP | CAKDS | RASQD | QASSL | CQQSY | QVQLVQSGAEVKKP | DIQMTQSPSSLSA |
| | YDIN | NSGNT | DYSNL | ITNDL | ES | TIPLT | GASVKVSCKASGYT | SVGDRVTITCRAS |
| | (SEQ | GYA | LWDYW | G | (SEQ | F | FTSYDINWVRQAPG | QDITNDLGWYQQK |
| | ID | (SEQ | (SEQ | (SEQ | ID | (SEQ | QGLEWMGWMNPNSG | PGKAPKLLIYQAS |
| | NO: | ID | ID | ID | NO: | ID | NTGYAQKFQGRVTM | SLESGVPSRFSGS |
| | 310) | NO: | NO: | NO: | 332) | NO: | TRDTSTSTVYMELS | GSGTDFTLTISSL |
| | | 205) | 330) | 331) | | 333) | SLRSEDTAVYYCAK | QPEDFATYYCQQS |
| | | | | | | | DSDYSNLLWDYWGQ | YTIPLTFGQGTKV |
| | | | | | | | GTLVTVSS (SEQ | EIK (SEQ ID |
| | | | | | | | ID NO: 551) | NO: 552) |
| 66. | YTFTG | GIINP | CARDG | RASQG | SNLET | QQYYS | QVQLVQSGAEVKKP | DIQMTQSPSSLSA |
| | HYMH | SGGST | AWFGE | ISNWL | (SEQ | FPLYT | GASVKVSCKASGYT | SVGDRVTITCRAS |
| | (SEQ | SYA | EYYYG | A | ID | (SEQ | FTGHYMHWVRQAPG | QGISNWLAWYQQK |
| | ID | (SEQ | MDVW | (SEQ | NO: | ID | QGLEWMGIINPSGG | PGKAPKLLIYDAS |
| | NO: | ID | (SEQ | ID | 491) | NO: | STSYAQKFQGRVTM | NLETGVPSRFSGS |
| | 244) | NO: | ID | NO: | | 555) | TRDTSTSTVYMELS | GSGTDFTLTISSL |
| | | 69) | NO: | 554) | | | SLRSEDTAVYYCAR | QPEDFATYYCQQY |
| | | | 553) | | | | DGAWFGEEYYYGMD | YSFPLYTFGQGTK |
| | | | | | | | VWGQGTTVTVSS | VEIK (SEQ ID |
| | | | | | | | (SEQ ID NO: | NO: 557) |
| | | | | | | | 556) | |
| 67. | YTFTG | GMIYP | CAMTG | RASQG | STLQS | QQSYS | QVQLVQSGAEVKKP | DIQMTQSPSSLSA |
| | YYMH | RDGST | WGYGM | INNYL | (SEQ | APPT | GASVKVSCKASGYT | SVGDRVTITCRAS |
| | (SEQ | SYA | DVW | A | ID | (SEQ | FTGYYMHWVRQAPG | QGINNYLAWYQQK |
| | ID | (SEQ | (SEQ | (SEQ | NO: | ID | QGLEWMGMIYPRDG | PGKAPKLLIYDAS |
| | NO: | ID | ID | ID | 420) | NO: | STSYAQKFQGRVTM | TLQSGVPSRFSGS |
| | 128) | NO: | NO: | NO: | | 561) | TRDTSTSTVYMELS | GSGTDFTLTISSL |
| | | 558) | 559) | 560) | | | SLRSEDTAVYYCAM | QPEDFATYYCQQS |
| | | | | | | | TGWGYGMDVWGKGT | YSAPPTFGQGTKL |
| | | | | | | | TVTVSS (SEQ ID | EIK (SEQ ID |
| | | | | | | | NO: 562) | NO: 563) |
| 68. | FTFGD | AVVSY | CAKDI | RASQN | DASNL | CQQAN | EVQLLESGGGLVQP | DIQMTQSPSSLSA |
| | YAMS | DGTNK | CSSTS | INNYV | ET | SFPPT | GGSLRLSCAASGFT | SVGDRVTITCRAS |
| | (SEQ | YYA | CYFDL | N | (SEQ | F | FGDYAMSWVRQAPG | QNINNYVNWYQQK |
| | ID | (SEQ | W | (SEQ | ID | (SEQ | KGLEWVAVVSYDGT | PGKAPKLLIYDAS |
| | NO: | ID | (SEQ | ID | NO: | ID | NKYYADSVKGRFTI | NLETGVPSRFSGS |
| | 233) | NO: | ID | NO: | 159) | NO: | SRDNSKNTLYLQMN | GSGTDFTLTISSL |
| | | 335) | NO: | 337) | | 338) | SLRAEDTAVYYCAK | QPEDFATYYCQQA |
| | | | 336) | | | | DICSSTSCYFDLWG | NSFPPTFGQGTRL |
| | | | | | | | RGTLVTVSS (SEQ | EIK (SEQ ID |
| | | | | | | | ID NO: 564) | NO: 565) |
| 69. | YTFTS | GIIDP | CAREE | RASQG | ATSSL | CQQTY | QVQLVQSGAEVKKP | DIQMTQSPSSLSA |
| | YYMH | SGGST | WSSGG | ISSYL | QT | SIPIT | GASVKVSCKASGYT | SVGDRVTITCRAS |
| | (SEQ | SYA | VGYFD | A | (SEQ | F | FTSYYMHWVRQAPG | QGISSYLAWYQQK |
| | ID | (SEQ | YW | (SEQ | ID | (SEQ | QGLEWMGIIDPSGG | PGKAPKLLIYATS |
| | NO: | ID | (SEQ | ID | NO: | ID | STSYAQKFQGRVTM | SLQTGVPSRFSGS |
| | 215) | NO: | ID | NO: | 343) | NO: | TRDTSTSTVYMELS | GSGTDFTLTISSL |
| | | 340) | NO: | 342) | | 344) | SLRSEDTAVYYCAR | QPEDFATYYCQQT |
| | | | 341) | | | | EEWSSGGVGYFDYW | YSIPITFGQGTRL |
| | | | | | | | GQGTLVTVSS | EIK (SEQ ID |
| | | | | | | | (SEQ ID NO: | NO: 567) |
| | | | | | | | 566) | |
| 70. | YPFTD | GWIKP | CARDR | RASQS | SSLQS | QQSYD | QVQLVQSGAEVKKP | DIQMTQSPSSLSA |
| | YYMH | NSGDT | FVGKP | ISVWL | (SEQ | TPYT | GASVKVSCKASGYP | SVGDRVTITCRAS |
| | (SEQ | EYA | DYYYY | A | ID | (SEQ | FTDYYMHWVRQAPG | QSISVWLAWYQQK |
| | ID | (SEQ | GMDVW | (SEQ | NO: | ID | QGLEWMGWIKPNSG | PGKAPKLLIYAAS |
| | NO: | ID | (SEQ | ID | 362) | NO: | DTEYAQKFQGRVTM | SLQSGVPSRFSGS |
| | 568) | NO: | ID | NO: | | 572) | TRDTSTSTVYMELS | GSGTDFTLTISSL |
| | | 569) | NO: | 571) | | | SLRSEDTAVYYCAR | QPEDFATYYCQQS |
| | | | 570) | | | | DRFVGKPDYYYYGM | YDTPYTFGQGTKL |
| | | | | | | | DVWGQGTMVTVSS | EIK (SEQ ID |
| | | | | | | | (SEQ ID NO: | NO: 574) |
| | | | | | | | 573) | |
| 71. | YTFTS | GIINP | CARDS | RASQG | AASSL | CQQSY | QVQLVQSGAEVKKP | DIQMTQSPSSLSA |
| | YYMH | SGGST | VAGTG | ISNYF | QG | SLPYT | GASVKVSCKASGYT | SVGDRVTITCRAS |
| | (SEQ | SYA | GRYYG | A | (SEQ | F | FTSYYMHWVRQAPG | QGISNYFAWYQQK |
| | ID | (SEQ | MDVW | (SEQ | ID | (SEQ | QGLEWMGIINPSGG | PGKAPKLLIYAAS |
| | NO: | ID | (SEQ | ID | NO: | ID | STSYAQKFQGRVTM | SLQGGVPSRFSGS |
| | 215) | NO: | ID | NO: | 352) | NO: | TRDTSTSTVYMELS | GSGTDFTLTISSL |
| | | 69) | NO: | 351) | | 353) | SLRSEDTAVYYCAR | QPEDFATYYCQQS |
| | | | 350) | | | | DSVAGTGGRYYGMD | YSLPYTFGQGTKL |
| | | | | | | | VWGQGTLVTVSS | EIK (SEQ ID |
| | | | | | | | (SEQ ID NO: | NO: 576) |
| | | | | | | | 575) | |
| 72. | YTFTS | GVINP | CASGA | RASQS | SYLAT | QQSYS | QVQLVQSGAEVKKP | DIQMTQSPSSLSA |
| | YYMH | IGGTT | PSYYY | ISSYL | (SEQ | TPLT | GASVKVSCKASGYT | SVGDRVTITCRAS |
| | (SEQ | TYA | YGMDV | N | ID | (SEQ | FTSYYMHWVRQAPG | QSISSYLNWYQQK |
| | ID | (SEQ | W | (SEQ | NO: | ID | QGLEWMGVINPIGG | PGKAPKLLIYGTS |
| | NO: | ID | (SEQ | ID | 579) | NO: | TTTYAQKFQGRVTM | YLATGVPSRFSGS |
| | 215) | NO: | ID | NO: | | 429) | TRDTSTSTVYMELS | GSGTDFTLTISSL |
| | | 577) | NO: | 172) | | | SLRSEDTAVYYCAS | QPEDFATYYCQQS |
| | | | 578) | | | | GAPSYYYYGMDVWG | YSTPLTFGQGTKV |
| | | | | | | | QGTLVTVSS (SEQ | EIK (SEQ ID |
| | | | | | | | ID NO: 580) | NO: 581) |
| 73. | YTFTS | GRINP | CARAG | QASQD | TALRT | QQSYS | QVQLVQSGAEVKKP | DIQMTQSPSSLSA |
| | NYVH | HSGDT | QLWSD | IRNYL | (SEQ | HPLT | GASVKVSCKASGYT | SVGDRVTITCQAS |
| | (SEQ | SYA | WYFDL | N | ID | (SEQ | FTSNYVHWVRQAPG | QDIRNYLNWYQQK |
| | ID | (SEQ | W | (SEQ | NO: | ID | QGLEWMGRINPHSG | PGKAPKLLIYAAT |
| | NO: | ID | (SEQ | ID | 585) | NO: | DTSYAQKFQGRVTM | ALRTGVPSRFSGS |
| | 582) | NO: | ID | NO: | | 586) | TRDTSTSTVYMELS | GSGTDFTLTISSL |
| | | 583) | NO: | 280) | | | SLRSEDTAVYYCAR | QPEDFATYYCQQS |
| | | | 584) | | | | AGQLWSDWYFDLWG | YSHPLTFGQGTKV |
| | | | | | | | RGTLVTVSS (SEQ | EIK (SEQ ID |
| | | | | | | | ID NO: 587) | NO: 588) |
| 74. | YTFTG | GIINP | CTTAD | RASQG | AASSL | CQQYY | QVQLVQSGAEVKKP | DIQMTQSPSSLSA |
| | YYMH | SGGNT | YYYYM | ISNYL | QS | SNADF | GASVKVSCKASGYT | SVGDRVTITCRAS |
| | (SEQ | KYA | DVW | A | (SEQ | (SEQ | FTGYYMHWVRQAPG | QGISNYLAWYQQK |
| | ID | (SEQ | (SEQ | (SEQ | ID | ID | QGLEWMGIINPSGG | PGKAPKLLIYAAS |
| | NO: | ID | ID | ID | NO: | NO: | NTKYAQKFQGRVTM | SLQSGVPSRFSGS |
| | 128) | NO: | NO: | NO: | 65) | 357) | TRDTSTSTVYMELS | GSGTDFTLTISSL |
| | | 355) | 356) | 218) | | | SLRSEDTAVYYCTT | QPEDFATYYCQQY |
| | | | | | | | ADYYYYMDVWGKGT | YSNADFGQGTKVE |
| | | | | | | | TVTVSS (SEQ ID | IK (SEQ ID |
| | | | | | | | NO: 589) | NO: 590) |
| 75. | FTFSD | SYISG | CARDR | RASQS | SSLQS | QQYKS | EVQLLESGGGLVQP | DIQMTQSPSSLSA |
| | FWMH | DSGYT | PYYYY | VSRSL | (SEQ | YPVT | GGSLRLSCAASGFT | SVGDRVTITCRAS |
| | (SEQ | NYA | MDVW | A | ID | (SEQ | FSDFWMHWVRQAPG | QSVSRSLAWYQQK |
| | ID | (SEQ | (SEQ | (SEQ | NO: | ID | KGLEWISYISGDSG | PGKAPKLLIYAAS |
| | NO: | ID | ID | ID | 362) | NO: | YTNYADSVKGRFTI | SLQSGVPSRFSGS |
| | 359) | NO: | NO: | NO: | | 363) | SRDNSKNTLYLQMN | GSGTDFTLTISSL |
| | | 170) | 360) | 361) | | | SLRAEDTAVYYCAR | QPEDFATYYCQQY |
| | | | | | | | DRPYYYYMDVWGKG | KSYPVTFGQGTKV |
| | | | | | | | TTVTVSS (SEQ | EIK (SEQ ID |
| | | | | | | | ID NO: 591) | NO: 592) |
| 76. | FTFDD | SDISG | CAKDV | QASQD | SYLQS | QQAHN | EVQLLESGGGLVQP | DIQMTQSPSSLSA |
| | YTMH | SGGST | VVAGT | ISNYL | (SEQ | YPIT | GGSLRLSCAASGFT | SVGDRVTITCQAS |
| | (SEQ | YYA | PLHFD | N | ID | (SEQ | FDDYTMHWVRQAPG | QDISNYLNWYQQK |
| | ID | (SEQ | YW | (SEQ | NO: | ID | KGLEWVSDISGSGG | PGKAPKLLIYAAS |
| | NO: | ID | (SEQ | ID | 368) | NO: | STYYADSVKGRFTI | YLQSGVPSRFSGS |
| | 365) | NO: | ID | NO: | | 369) | SRDNSKNTLYLQMN | GSGTDFTLTISSL |
| | | 366) | NO: | 138) | | | SLRAEDTAVYYCAK | QPEDFATYYCQQA |
| | | | 367) | | | | DVVVAGTPLHFDYW | HNYPITFGQGTRL |
| | | | | | | | GQGTLVTVSS | EIK (SEQ ID |
| | | | | | | | (SEQ ID NO: | NO: 594) |
| | | | | | | | 593) | |
| 77. | FTFSN | ASISS | CAREV | RASQS | SSLQS | QQANA | EVQLLESGGGLVQP | DIQMTQSPSSLSA |
| | AWMS | TSAYI | VGATT | ISTWL | (SEQ | FPPT | GGSLRLSCAASEFT | SVGDRVTITCRAS |
| | (SEQ | DYA | FDYW | A | ID | (SEQ | FSNAWMSWVRQAPG | QSISTWLAWYQQK |
| | ID | (SEQ | (SEQ | (SEQ | NO: | ID | KGLEWVASISSTSA | PGKAPKLLIYAAS |
| | NO: | ID | ID | ID | 362) | NO: | YIDYADSVKGRFTI | SLQSGVPSRFSGS |
| | 183) | NO: | NO: | NO: | | 374) | SRDNSKNTLYLQMN | GSGTDFTLTISSL |
| | | 371) | 372) | 373) | | | SLRAEDTAVYYCAR | QPEDFATYYCQQA |
| | | | | | | | EVVGATTFDYWGQG | NAFPPTFGQGTRL |
| | | | | | | | TLVTVSS (SEQ | EIK (SEQ ID |
| | | | | | | | ID NO: 595) | NO: 596) |
| 78. | GTFSS | GWMEP | CAKGG | KSSQS | STRES | QQYYS | QVQLVQSGAEVKKP | DIVMTQSPDSLAV |
| | YAIS | HTGNT | FSWFD | VLYSS | (SEQ | TPPT | GSSVKVSCKASGGT | SLGERATINCKSS |
| | (SEQ | RYA | PW | NNKNY | ID | (SEQ | FSSYAI SWVRQAPG | QSVLYSSNNKNYL |
| | ID | (SEQ | (SEQ | LA | NO: | ID | QGLEWMGWMEPHTG | AWYQQKPGQPPKL |
| | NO: | ID | ID | (SEQ | 378) | NO: | NTRYAQKFQGRVTI | LIYWASTRESGVP |
| | 77) | NO: | NO: | ID | | 379) | TADESTSTAYMELS | DRFSGSGSGTDFT |
| | | 376) | 377) | NO: | | | SLRSEDTAVYYCAK | LTISSLQAEDVAV |
| | | | | 289) | | | GGFSWFDPWGQGTL | YYCQQYYSTPPTF |
| | | | | | | | VTVSS (SEQ ID | GQGTRLEIK |
| | | | | | | | NO: 597) | (SEQ ID NO: |
| | | | | | | | | 598) |
| 79. | FTFDD | ASITS | CARER | RASQG | STRAT | QQYYT | EVQLLESGGGLVKP | EIVMTQSPATLSV |
| | YAMH | SSAFI | VDWNS | ISNSY | (SEQ | YPPT | GGSLRLSCAASGFT | SPGERATLSCRAS |
| | (SEQ | DYA | YFDLW | LA | ID | (SEQ | FDDYAMHWVRQAPG | QGISNSYLAWYQQ |
| | ID | (SEQ | (SEQ | (SEQ | NO: | ID | KGLEWVASITSSSA | KPGQAPRLLIYGA |
| | NO: | ID | ID | ID | 384) | NO: | FIDYAASVKGRFTI | STRATGIPARFSG |
| | 135) | NO: | NO: | NO: | | 385) | SRDDSKNTLYLQMN | SGSGTEFTLTISS |
| | | 381) | 382) | 383) | | | SLKTEDTAVYYCAR | LQSEDFAVYYCQQ |
| | | | | | | | ERVDWNSYFDLWGR | YYTYPPTFGPGTK |
| | | | | | | | GTLVTVSS (SEQ | VDIK (SEQ ID |
| | | | | | | | ID NO: 599) | NO: 600) |
| 80. | FTFDD | SAISG | CAKDL | QASQD | SNLEA | QQSYS | EVQLLESGGGLVQP | DIQMTQSPSSLSA |
| | YAMH | SGGST | GVVVP | ISNHL | (SEQ | TPLT | GGSLRLSCAASGFT | SVGDRVTITCQAS |
| | (SEQ | YYA | AALDY | N | ID | (SEQ | FDDYAMHWVRQAPG | QDISNHLNWYQQK |
| | ID | (SEQ | W | (SEQ | NO: | ID | KGLEWVSAISGSGG | PGKAPKLLIYDAS |
| | NO: | ID | (SEQ | ID | 603) | NO: | STYYADSVKGRFTI | NLEAGVPSRFSGS |
| | 135) | NO: | ID | NO: | | 429) | SRDNSKNTLYLQMN | GSGTDFTLTISSL |
| | | 103) | NO: | 602) | | | SLRAEDTAVYYCAK | QPEDFATYYCQQS |
| | | | 601) | | | | DLGVVVPAALDYWG | YSTPLTFGGGTKV |
| | | | | | | | QGTTVTVSS (SEQ | EIK (SEQ ID |
| | | | | | | | ID NO: 604) | NO: 605) |
| 81. | FAFSS | AGTSG | CARET | RASQG | ANLEG | QQSDI | EVQLLESGGGLVKP | DIQMTQSPSSLSA |
| | HWMH | SGESR | YYYYY | ISNYL | (SEQ | FPPT | GGSLRLSCAASGFA | SVGDRVTITCRAS |
| | (SEQ | DYA | MDVW | A | ID | (SEQ | FSSHWMHWVRQAPG | QGISNYLAWYQQK |
| | ID | (SEQ | (SEQ | (SEQ | NO: | ID | KGLEWVAGTSGSGE | PGKAPKLLIYDAA |
| | NO: | ID | ID | ID | 390) | NO: | SRDYADFVKGRFTI | NLEGGVPSRFSGS |
| | 387) | NO: | NO: | NO: | | 391) | SRDDSKNTLYLQMN | GSGTDFTLTISSL |
| | | 388) | 389) | 218) | | | SLKTEDTAVYYCAR | QPEDFATYYCQQS |
| | | | | | | | ETYYYYYMDVWGKG | DIFPPTFGQGTKV |
| | | | | | | | TTVTVSS (SEQ | EIK (SEQ ID |
| | | | | | | | ID NO: 606) | NO: 607) |
| 82. | YTFTR | GWINV | CARES | RASQS | SSLQS | QQSNS | QVQLVQSGAEVKKP | DIQMTQSPSSLSA |
| | HWIH | KTGGA | SGWYG | ISNYL | (SEQ | FPLT | GASVKVSCKASGYT | SVGDRATITCRAS |
| | (SEQ | GYA | TDVW | A | ID | (SEQ | FTRHWIHWVRQAPG | QSISNYLAWYQQK |
| | ID | (SEQ | (SEQ | (SEQ | NO: | ID | QGLEWMGWINVKTG | PGKAPKLLIYAAS |
| | NO: | ID | ID | ID | 362) | NO: | GAGYAQKFQGRVTM | SLQSGVPSRFSGS |
| | 393) | NO: | NO: | NO: | | 397) | TRDTSTSTVYMELS | GSGTDFTLTISSL |
| | | 394) | 395) | 396) | | | SLRSEDTAVYYCAR | QPEDFATYYCQQS |
| | | | | | | | ESSGWYGTDVWGQG | NSFPLTFGGGTKV |
| | | | | | | | TTVTVSS (SEQ | EIK (SEQ ID |
| | | | | | | | ID NO: 608) | NO: 609) |
| 83. | FTFSS | AAISY | CAREN | QASQD | NLRS | QQANS | EVQLLESGGGLVQP | DIQMTQSPSSLSA |
| | YWMH | DGKYK | KQWLA | ISNFV | (SEQ | FPVT | GGSLRLSCAASGFT | SVGDRVTITCQAS |
| | (SEQ | DYE | SFDYW | N | ID | (SEQ | FSSYWMHWVRQAPG | QDISNFVNWYQQK |
| | ID | (SEQ | (SEQ | (SEQ | NO: | ID | KGLEWVAAISYDGK | PGKAPKLLIYAAN |
| | NO: | ID | ID | ID | 402) | NO: | YKDYEDSVKGRFTI | LRSGVPSRFSGSG |
| | 83) | NO: | NO: | NO: | | 403) | SRDNSKNTLYLQMN | SGTDFTLTISSLQ |
| | | 399) | 400) | 401) | | | SLRAEDTAVYYCAR | PEDFATYYCQQAN |
| | | | | | | | ENKQWLASFDYWGQ | SFPVTFGPGTKVD |
| | | | | | | | GTLVTVSS (SEQ | IK (SEQ ID |
| | | | | | | | ID NO: 610) | NO: 611) |
| 84. | GTFSS | GWISA | CASRV | QASEH | SSLQS | QQTDS | QVQLVQSGAEVKKP | DIQMTQSPSSLSA |
| | SAIS | YNGYT | HSGGS | IYNYL | (SEQ | IPIT | GASVKVSCKASGGT | SVGDRVTITCQAS |
| | (SEQ | NYA | YPDDY | N | ID | (SEQ | FSSSAISWVRQAPG | EHIYNYLNWYQQK |
| | ID | (SEQ | W | (SEQ | NO: | ID | QGLEWMGWISAYNG | PGKAPKLLIYAAS |
| | NO: | ID | (SEQ | ID | 362) | NO: | YTNYAQKFQGRVTM | SLQSGVPSRESGS |
| | 612) | NO: | ID | NO: | | 616) | TRDTSTSTVYMELS | GSGTDFTLTISSL |
| | | 613) | NO: | 615) | | | SLRSEDTAVYYCAS | QPEDFATYYCQQT |
| | | | 614) | | | | RVHSGGSYPDDYWG | DSIPITFGQGTKV |
| | | | | | | | QGTLVTVSS (SEQ | EIK (SEQ ID |
| | | | | | | | ID NO: 617) | NO: 618) |

In some embodiments, the antibody comprises the CDRs of Clone ID: 6, Clone ID: 75, or Clone ID: 79 of Table 7.

The IgG and scFv formats illustrated herein are simply non-limiting examples. The CDRs provided herein can be placed in different formats, including different VH and VL/VK formats and still be able to bind to MAdCAM.

Although the CDRs are illustrated in the tables provided herein, there are other ways to annotate or identify CDRs. For example, in some embodiments, the HCDR2 can have an extra amino acid at the N-terminus. For example, for the HCDR2 of Clone 6 the table indicates that it has a sequence of: SRLINSYGTSTTYA (SEQ ID NO: 91) However, in some embodiments, the HCDR2 has a sequence of VSRINSYGTSTTYA (SEQ ID NO: 629), which is shown with an extra residue, a valine, at the N-terminus of the HCDR2. The valine is clearly illustrated in VH peptide of the tables provided herein. Therefore, in some embodiments, the HCDR2 comprises one additional amino acid immediately to the N-terminus of the HCDR2 listed in the table. The residue would be the residue that is immediately to the N-terminus of the HCDR2 found in the VH sequence provided for in the table in the same row. One of skill in the art with this information could immediately envisage the HCDR2 peptide sequence that has the additional amino acid residue immediately to the N-terminus of the HCDR2 listed in the table. These embodiments are sufficiently described and do not require application to list each of these different annotations and one of skill in the art with the guidance and description provided herein could write them out individually without any undue experimentation.

Similarly, the HCDR3 can exclude the cysteine residue. Each of the HCDR3 polypeptides provided for in Tables 6 and 7 begins with a cysteine residue. In some embodiments, the HCDR3 does not include the cysteine. Furthermore, in some embodiments, the HCDR3 does not have the last C-terminal residue illustrated in Table 6 and 7 provided for herein. Therefore, in some embodiments, the HCDR3 does not have the cysteine and/or the last C-terminal residue illustrated in the tables. One of skill in the art with this information could immediately envisage the HCDR3 peptide sequence that does not have the cysteine and/or the last C-terminal residue illustrated in the tables. These embodiments are sufficiently described and do not require application to list each of these different annotations and one of skill in the art with the guidance and description provided herein could write them out individually without any undue experimentation.

In some embodiments, the light chain CDR2 can have one or two extra amino acid residues at the N-terminus. These additional residues would be those that are immediately to the N-terminus of the light chain CDR2 (LCDR2) present in the VL/VK chain provided for herein, in the same row as the CDRs that are listed. For example, the LCDR2 of Clone 6 is provided as GASSLQS (SEQ ID NO: 87), but in some embodiments could be IYGASSLQS (SEQ ID NO: 630) or YGASSLQS (SEQ ID NO: 631). One of skill in the art with this information could immediately envisage the LCDR2 peptide sequence that has one or two extra amino acid residues at the N-terminus of the LCDR2 sequence provided for herein. These embodiments are sufficiently described and do not require application to list each of these different annotations and one of skill in the art with the guidance and description provided herein could write them out individually without any undue experimentation.

There are also alternative systems for annotating CDRs, any of which can be used. For example, CDRs can be chosen based on the Kabat sytem, the IMGT system, or the Chothia system. Other proprietary systems can also be used, which may be based on the predicted 3-dimensional structure of the protein. Accordingly, in some embodiments, the CDRs of Clone ID: 6, Clone ID: 75, or Clone ID: 79 of Table 7 can also be characterized as shown in Table 8. These alternative CDRs can be substituted for these clone referenced in Table 7 or the equivalent clone numbering in Table 6, i.e., Clone 6, Clone 59, and Clone 63.

TABLE 8
Alterative CDRs for Certain Clones

| Clone No. | Annotation | | | | | | |
|---|---|---|---|---|---|---|---|
| (Table 7) | System | LCDR1 | LCDR2 | LCDR3 | HCDR1 | HCDR2 | HCDR3 |
| 6 | Proprietary | RASQIIG | SSLQS | QQSYRLP | FTFNNYA | SRINSYG | CAREGPVA |
| | | TNLA | (SEQ ID | FT (SEQ | FH (SEQ | TSTTYA | GYWYFDLW |
| | | (SEQ ID | NO: | ID NO: | ID NO: | (SEQ ID | (SEQ ID |
| | | NO: 93) | 362) | 632) | 90) | NO: 91) | NO: 92) |
| | Other | RASQIIG | GASSLQS | COQSYRL | FTENNYA | SRINSYG | CAREGPVA |
| | Annotation | TNLA | (SEQ ID | PFTF | FH (SEQ | TSTTYA | GYWYFDLW |
| | | (SEQ ID | NO: 87) | (SEQ ID | ID NO: | (SEQ ID | (SEQ ID |
| | | NO: 93) | | NO: 94) | 90 | NO: 91) | NO: 92) |
| | Kabat | RASQIIG | GASSLQS | QQSYRLP | NYAFH | RINSYGT | EGPVAGYW |
| | | TNLA | (SEQ ID | FT (SEQ | (SEQ ID | STTYADS | YFDL |
| | | (SEQ ID | NO: 87) | ID NO: | NO: | VKG | (SEQ ID |
| | | NO: 93) | | 632) | 633) | (SEQ ID | NO: 635) |
| | | | | | | NO: | |
| | | | | | | 634) | |
| | IMGT | QIIGTN | GAS | QQSYRLP | GFTFNNY | INSYGTS | AREGPVAG |
| | | (SEQ ID | | FT (SEQ | A (SEQ | T (SEQ | YWYFDL |
| | | NO: | | ID NO: | ID NO: | ID NO: | (SEQ ID |
| | | 636) | | 632) | 637) | 638) | NO: 639) |
| | CHOTHIA | RASQIIG | GASSLQS | QQSYRLP | GFTENNY | NSYGTS | EGPVAGYW |
| | | TNLA | (SEQ ID | FT (SEQ | (SEQ ID | (SEQ ID | YFDL |
| | | (SEQ ID | NO: 87) | ID NO: | NO: | NO: | (SEQ ID |
| | | NO: 93) | | 632) | 640) | 641) | NO: 635) |
| 75 | Proprietary | RASQSVS | SSLQS | QQYKSYP | FTFSDFW | SYISGDS | CARDRPYY |
| | | RSLA | (SEQ ID | VT (SEQ | MH (SEQ | GYTNYA | YYMDVW |
| | | (SEQ ID | NO: | ID NO: | ID NO: | (SEQ ID | (SEQ ID |
| | | NO: | 362) | 363) | 359) | NO: | NO: 360) |
| | | 361) | | | | 170) | |
| | Other | RASQSVS | AASSLQS | COQYKSY | FTFSDFW | SYISGDS | CARDRPYY |
| | Annotation | RSLA | (SEQ ID | PVTF | MH (SEQ | GYTNYA | YYMDVW |
| | | (SEQ ID | NO: 65) | (SEQ ID | ID NO: | (SEQ ID | (SEQ ID |
| | | NO: | | NO: | 359) | NO: | NO: 360) |
| | | 361) | | 642) | | 170) | |
| | Kabat | RASQSVS | AASSLQS | QQYKSYP | DFWMH | YISGDSG | DRPYYYYM |
| | | RSLA | (SEQ ID | VT (SEQ | (SEQ ID | YTNYADS | DV (SEQ |
| | | (SEQ ID | NO: 65) | ID NO: | NO: | VKG | ID NO: |
| | | NO: | | 363) | 643) | (SEQ ID | 645) |
| | | 361) | | | | NO: | |
| | | | | | | 644) | |
| | IMGT | QSVSRS | AAS | QQYKSYP | GFTFSDE | ISGDSGY | ARDRPYYY |
| | | (SEQ ID | | VT (SEQ | W (SEQ | T (SEQ | YMDV |
| | | NO: | | ID NO: | ID NO: | ID NO: | (SEQ ID |
| | | 646) | | 363) | 647) | 648) | NO: 649) |
| | CHOTHIA | RASQSVS | AASSLQS | QQYKSYP | GFTFSDE | SGDSGY | DRPYYYYM |
| | | RSLA | (SEQ ID | VT (SEQ | (SEQ ID | (SEQ ID | DV (SEQ |
| | | (SEQ ID | NO: 65) | ID NO: | NO: | NO: | ID NO: |
| | | NO: | | 363) | 650) | 651) | 645) |
| | | 361) | | | | | |
| 79 | Proprietary | RASQGIS | STRAT | QQYYTYP | FTFDDYA | ASITSSS | CARERVDW |
| | | NSYLA | (SEQ ID | PT (SEQ | MH (SEQ | AFIDYA | NSYFDLW |
| | | (SEQ ID | NO: | ID NO: | ID NO: | (SEQ ID | (SEQ ID |
| | | NO: | 384) | 385) | 135) | NO: | NO: 382) |
| | | 383) | | | | 381) | |
| | Other | RASQGIS | GASTRAT | COQYYTY | FTEDDYA | ASITSSS | CARERVDW |
| | Annotation | NSYLA | (SEQ ID | PPTF | MH (SEQ | AFIDYA | NSYFDLW |
| | | (SEQ ID | NO: | (SEQ ID | ID NO: | (SEQ ID | (SEQ ID |
| | | NO: | 241) | NO: | 135) | NO: | NO: 382) |
| | | 383) | | 652) | | 381) | |
| | Kabat | RASQGIS | GASTRAT | QQYYTYP | DYAMH | SITSSSA | ERVDWNSY |
| | | NSYLA | (SEQ ID | PT (SEQ | (SEQ ID | FIDYAAS | FDL (SEQ |
| | | (SEQ ID | NO: | ID NO: | NO: | VKG | ID NO: |
| | | NO: | 241) | 385) | 653) | (SEQ ID | 655) |
| | | 383) | | | | NO: | |
| | | | | | | 654) | |
| | IMGT | QGISNSY | GAS | QQYYTYP | GFTEDDY | ITSSSAF | ARERVDWN |
| | | (SEQ ID | | PT (SEQ | A (SEQ | I (SEQ | SYFDL |
| | | NO: | | ID NO: | ID NO: | ID NO: | (SEQ ID |
| | | 656) | | 385) | 657) | 658) | NO: 659) |
| | CHOTHIA | RASQGIS | GASTRAT | QQYYTYP | GFTEDDY | TSSSAF | ERVDWNSY |
| | | NSYLA | (SEQ ID | PT (SEQ | (SEQ ID | (SEQ ID | FDL (SEQ |
| | | (SEQ ID | NO: | ID NO: | NO: | NO: | ID NO: |
| | | NO: | 241) | 385) | 660) | 661) | 655) |
| | | 383) | | | | | |

In some embodiments, the MAdCAM antibody is selected from the following table

TABLE 9

| Clone | | | | | | | | |
|---|---|---|---|---|---|---|---|---|
| (Fab) | VH Seq | VK Seq | HCDR1 | HCDR2 | HCDR3 | LCDR1 | LCDR2 | LCDR3 |
| MIAB128 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PNYYY | VSRSL | (SEQ | YPVT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 1399) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PNYYYMDVWG | YKSYPVTFG | | | | | | |
| | KGTTVTVSS | QGTKVEIK | | | | | | |
| | (SEQ ID | (SEQ ID | | | | | | |
| | NO: 1346) | NO: 592) | | | | | | |
| MIAB128A | EVQLLESGGG | DIQMTQSPS | FTFSD | SYISG | CARDR | RASQS | SSLQS | QQYKS |
| | LVQPGGSLRL | SLSASVGDR | FWMH | DSGYT | PQYYY | VSRSL | (SEQ | YPVT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 1400) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PQYYYMDVWG | YKSYPVTFG | | | | | | |
| | KGTTVTVSS | QGTKVEIK | | | | | | |
| | (SEQ ID | (SEQ ID | | | | | | |
| | NO: 1348) | NO: 592) | | | | | | |
| MIAB129 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PGYYY | VSRSL | (SEQ | YPVT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 1401) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PGYYYMDVWG | YKSYPVTFG | | | | | | |
| | KGTTVTVSS | QGTKVEIK | | | | | | |
| | (SEQ ID | (SEQ ID | | | | | | |
| | NO: 1349) | NO: 592) | | | | | | |
| MIAB130 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYQ | VSRSL | (SEQ | YPVT |
| | SCAASGFTES | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 1402) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYQMDVWG | YKSYPVTFG | | | | | | |
| | KGTTVTVSS | QGTKVEIK | | | | | | |
| | (SEQ ID | (SEQ ID | | | | | | |
| | NO: 1350) | NO: 592) | | | | | | |
| MIAB131 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYG | VSRSL | (SEQ | YPVT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 1403) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYGMDVWG | YKSYPVTFG | | | | | | |
| | KGTTVTVSS | QGTKVEIK | | | | | | |
| | (SEQ ID | (SEQ ID | | | | | | |
| | NO: 1351) | NO: 592) | | | | | | |
| MIAB132 | EVQLLESGGG | DIQMTQSPS | FTFSN | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTFS | VTITCRASQ | (SEQ | NYA | MDVW | A | ID | (SEQ |
| | NFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 1404) | NO: | NO: | NO: | | 363) |
| | ADSVKGRFTI | QSGVPSRES | | 170) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1352) | NO: 592) | | | | | | |
| MIAB133 | EVQLLESGGG | DIQMTQSPS | FTFSS | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTFS | VTITCRASQ | (SEQ | NYA | MDVW | A | ID | (SEQ |
| | SFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 1405) | NO: | NO: | NO: | | 363) |
| | ADSVKGRFTI | QSGVPSRES | | 170) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1353) | NO: 592) | | | | | | |
| MIAB134 | EVQLLESGGG | DIQMTQSPS | FTFSA | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTFS | VTITCRASQ | (SEQ | NYA | MDVW | A | ID | (SEQ |
| | AFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 1406) | NO: | NO: | NO: | | 363) |
| | ADSVKGRFTI | QSGVPSRFS | | 170) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1354) | NO: 592) | | | | | | |
| MIAB135 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 361) | | |
| | SRDNSNNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1355) | NO: 592) | | | | | | |
| MIAB136 | EVQLLESGGG | DIQMTQSPS | FTFSS | SYISG | CARDR | RASQS | SSLQS | QQYKS |
| | LVQPGGSLRL | SLSASVGDR | YAMS | DSGYT | PYYYY | VSRSL | (SEQ | YPVT |
| | SCAASGFTFS | VTITCRASQ | (SEQ | NYA | MDVW | A | ID | (SEQ |
| | SYAMSWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 473) | NO: | NO: | NO: | | 363) |
| | ADSVKGRFTI | QSGVPSRES | | 170) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1356) | NO: 592) | | | | | | |
| MIAB137 | EVQLLESGGG | DIQMTQSPS | FTFSD | SAISG | CARDR | RASQS | SSLQS | QQYKS |
| | LVQPGGSLRL | SLSASVGDR | FWMH | SGGST | PYYYY | VSRSL | (SEQ | YPVT |
| | SCAASGFTES | VTITCRASQ | (SEQ | YYA | MDVW | A | ID | (SEQ |
| | DFWMHWVRQA | SVSRSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 359) | NO: | NO: | NO: | | 363) |
| | ADSVKGRFTI | QSGVPSRES | | 103) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1357) | NO: 592) | | | | | | |
| MIAB138 | EVQLLESGGG | DIQMTQSPS | FTFSS | SAISG | CARDR | RASOS | SSLQS | QQYKS |
| | LVQPGGSLRL | SLSASVGDR | YAMS | SGGST | PYYYY | VSRSL | (SEQ | YPVT |
| | SCAASGFTES | VTITCRASQ | (SEQ | YYA | MDVW | A | ID | (SEQ |
| | SYAMSWVRQA | SVSRSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 473) | NO: | NO: | NO: | | 363) |
| | ADSVKGRFTI | QSGVPSRFS | | 103) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1358) | NO: 592) | | | | | | |
| MIAB139 | EVQLLESGGG | DIQMTQSPS | FTFSD | SYISG | CARDR | RASQS | SSLQS | QQSYS |
| | LVQPGGSLRL | SLSASVGDR | FWMH | DSGYT | PYYYY | ISSYL | (SEQ | TPRT |
| | SCAASGFTES | VTITCRASQ | (SEQ | NYA | MDVW | N | ID | (SEQ |
| | DFWMHWVRQA | SISSYLNWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 1407) |
| | ADSVKGRFTI | QSGVPSRES | | 170) | 360) | 172) | | |
| | SRDNSKNTLY | GSGSGTDFT | | | | | | |
| | LQMNSLRAED | LTISSLQPE | | | | | | |
| | TAVYYCARDR | DEATYYCQQ | | | | | | |
| | PYYYYMDVWG | SYSTPRTFG | | | | | | |
| | KGTTVTVSS | QGTKVEIK | | | | | | |
| | (SEQ ID | (SEQ ID | | | | | | |
| | NO: 591) | NO: 1360) | | | | | | |
| MIAB140 | EVQLLESGGG | DIQMTQSPS | FTFSS | SAISG | CARDR | RASQS | SSLQS | QQSYS |
| | LVQPGGSLRL | SLSASVGDR | YAMS | SGGST | PYYYY | ISSYL | (SEQ | TPRT |
| | SCAASGFTES | VTITCRASQ | (SEQ | YYA | MDVW | N | ID | (SEQ |
| | SYAMSWVRQA | SISSYLNWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 473) | NO: | NO: | NO: | | 1407) |
| | ADSVKGRFTI | QSGVPSRES | | 103) | 360) | 172) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | SYSTPRTFG | | | | | | |
| | KGTTVTVSS | QGTKVEIK | | | | | | |
| | (SEQ ID | (SEQ ID | | | | | | |
| | NO: 1358) | NO: 1360) | | | | | | |
| MIAB141 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | ISSYL | (SEQ | YPVT |
| | SCAASGFTFS | VTITCRASQ | (SEQ | NYA | MDVW | N | ID | (SEQ |
| | DFWMHWVRQA | SISSYLNWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 172) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1361) | | | | | | |
| MIAB142 | EVQLLESGGG | DIQMTQSPS | FTFSD | SYISG | CARDR | RASQS | SSLQS | QQSYS |
| | LVQPGGSLRL | SLSASVGDR | FWMH | DSGYT | PYYYY | VSRSL | (SEQ | TPRT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 1407) |
| | ADSVKGRFTI | QSGVPSRFS | | 170) | 360) | 361) | | |
| | SRDNSKNTLY | GSGSGTDFT | | | | | | |
| | LQMNSLRAED | LTISSLQPE | | | | | | |
| | TAVYYCARDR | DFATYYCOQ | | | | | | |
| | PYYYYMDVWG | SYSTPRTFG | | | | | | |
| | KGTTVTVSS | QGTKVEIK | | | | | | |
| | (SEQ ID | (SEQ ID | | | | | | |
| | NO: 591) | NO: 1362) | | | | | | |
| MIAB143 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTES | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 592) | | | | | | |
| MIAB144 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 361) | | |
| | SRDNSKNTLY | GSGSGTDFT | | | | | | |
| | LQMNSLRAED | LTISSLQPE | | | | | | |
| | TAVYYCARDR | DFATYYCOQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 592) | | | | | | |
| MIAB145 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | ISRSL | (SEQ | YPVT |
| | SCAASGFTFS | VTITCRASQ | (SEQ | NYA | MDVW | A | ID | (SEQ |
| | DFWMHWVRQA | SISRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 1408) | | |
| | SRDNSKNTLY | GSGSGTDET | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1363) | | | | | | |
| MIAB146 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSSSL | (SEQ | YPVT |
| | SCAASGFTES | VTITCRASQ | (SEQ | NYA | MDVW | A | ID | (SEQ |
| | DFWMHWVRQA | SVSSSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 1409) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1364) | | | | | | |
| MIAB147 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRYL | (SEQ | YPVT |
| | SCAASGFTES | VTITCRASQ | (SEQ | NYA | MDVW | A | ID | (SEQ |
| | DFWMHWVRQA | SVSRYLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 1410) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1365) | | | | | | |
| MIAB148 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTES | VTITCRASQ | (SEQ | NYA | MDVW | N | ID | (SEQ |
| | DFWMHWVRQA | SVSRSLNWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 1411) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1366) | | | | | | |
| MIAB149 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | ISSSL | (SEQ | YPVT |
| | SCAASGFTFS | VTITCRASQ | (SEQ | NYA | MDVW | A | ID | (SEQ |
| | DFWMHWVRQA | SISSSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 1412) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1367) | | | | | | |
| MIAB150 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | ISRYL | (SEQ | YPVT |
| | SCAASGFTES | VTITCRASQ | (SEQ | NYA | MDVW | A | ID | (SEQ |
| | DFWMHWVRQA | SISRYLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 1413) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1368) | | | | | | |
| MIAB151 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | ISRSL | (SEQ | YPVT |
| | SCAASGFTFS | VTITCRASQ | (SEQ | NYA | MDVW | N | ID | (SEQ |
| | DFWMHWVRQA | SISRSLNWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 1414) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1369) | | | | | | |
| MIAB152 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSSYL | (SEQ | YPVT |
| | SCAASGFTES | VTITCRASQ | (SEQ | NYA | MDVW | A | ID | (SEQ |
| | DFWMHWVRQA | SVSSYLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 1415) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1370) | | | | | | |
| MIAB153 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSSSL | (SEQ | YPVT |
| | SCAASGETFS | VTITCRASQ | (SEQ | NYA | MDVW | N | ID | (SEQ |
| | DFWMHWVRQA | SVSSSLNWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 1416) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSSV | QGTKVEIK | | | | | | |
| | (SEQ ID | (SEQ ID | | | | | | |
| | NO: 1548) | NO: 1371) | | | | | | |
| MIAB154 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRYL | (SEQ | YPVT |
| | SCAASGFTFS | VTITCRASQ | (SEQ | NYA | MDVW | N | ID | (SEQ |
| | DFWMHWVRQA | SVSRYLNWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 1417) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1372) | | | | | | |
| MIAB155 | EVQLLESGGG | DIQMTQSPS | FTFSD | SYISG | CARDR | RASQS | SSLQS | ΩQYKS |
| | LVQPGGSLRL | SLSASVGDR | FWMH | DSGYT | PYYYY | ISSSL | (SEQ | YPVT |
| | SCAASGFTES | VTITCRASQ | (SEQ | NYA | MDVW | N | ID | (SEQ |
| | DFWMHWVRQA | SISSSLNWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 1418) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1373) | | | | | | |
| MIAB156 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | ISRYL | (SEQ | YPVT |
| | SCAASGFTFS | VTITCRASQ | (SEQ | NYA | MDVW | N | ID | (SEQ |
| | DFWMHWVRQA | SISRYLNWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 1419) | | |
| | SRDNSKNTLY | GSGSGTDFT | | | | | | |
| | LQMNSLRAED | LTISSLQPE | | | | | | |
| | TAVYYCARDR | DFATYYCOQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1374) | | | | | | |
| MIAB157 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | ISSYL | (SEQ | YPVT |
| | SCAASGFTES | VTITCRASQ | (SEQ | NYA | MDVW | A | ID | (SEQ |
| | DFWMHWVRQA | SISSYLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 1420) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1375) | | | | | | |
| MIAB158 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSSYL | (SEQ | YPVT |
| | SCAASGFTES | VTITCRASQ | (SEQ | NYA | MDVW | N | ID | (SEQ |
| | DFWMHWVRQA | SVSSYLNWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 1421) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1376) | | | | | | |
| MIAB159 | EVQLLESGGG | DIQMTQSPS | FTFSD | SYISG | CARDR | RASQS | SSLQS | QQYKS |
| | LVQPGGSLRL | SLSASVGDR | FWMH | ESGYT | PYYYY | VSRSL | (SEQ | YPVT |
| | SCAASGFTES | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGESGYTNY | LLIYAASSL | 359) | NO: | NO: | NO: | | 363) |
| | ADSVKGRFTI | QSGVPSRFS | | 1422) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1377) | NO: 592) | | | | | | |
| MIAB160 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTES | VTITCRASQ | (SEQ | QYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTQY | LLIYAASSL | 359) | NO: | NO: | NO: | | 363) |
| | ADSVKGRFTI | QSGVPSRES | | 1423) | 360) | 361) | | |
| | SRDNSKNTLY | GSGSGTDFT | | | | | | |
| | LQMNSLRAED | LTISSLQPE | | | | | | |
| | TAVYYCARDR | DFATYYCOQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1378) | NO: 592) | | | | | | |
| MIAB161 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 361) | | |
| | SRDQSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1379) | NO: 592) | | | | | | |
| MIAB162 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTFS | VTITCRASQ | (SEQ | NYA | MDVW | G | ID | (SEQ |
| | DFWMHWVRQA | SVSRSLGWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 1424) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1380) | | | | | | |
| MIAB163 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTFS | VTITCRASQ | (SEQ | NYA | MDVW | D | ID | (SEQ |
| | DFWMHWVRQA | SVSRSLDWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 1425) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1381) | | | | | | |
| MIAB164 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTES | VTITCRASQ | (SEQ | NYA | MDVW | T | ID | (SEQ |
| | DFWMHWVRQA | SVSRSLTWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 1426) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1382) | | | | | | |
| MIAB165 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTES | VTITCRASQ | (SEQ | NYA | MDVW | S | ID | (SEQ |
| | DFWMHWVRQA | SVSRSLSWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 1427) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1383) | | | | | | |
| MIAB166 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTES | VTITCRASQ | (SEQ | NYA | MDVW | E | ID | (SEQ |
| | DFWMHWVRQA | SVSRSLEWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 1428) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1384) | | | | | | |
| MIAB167 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTFS | VTITCRASQ | (SEQ | NYA | MDVW | K | ID | (SEQ |
| | DFWMHWVRQA | SVSRSLKWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 1429) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1385) | | | | | | |
| MIAB168 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTES | VTITCRASQ | (SEQ | NYA | MDVW | L | ID | (SEQ |
| | DFWMHWVRQA | SVSRSLLWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 360) | 1430) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 1386) | | | | | | |
| MIAB169 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYI | VSRSL | (SEQ | YPVT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 1431) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1387) | NO: 592) | | | | | | |
| MIAB170 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYW | VSRSL | (SEQ | YPVT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 1432) | 361) | | |
| | SRDNSKNTLY | GSGSGTDET | | | | | | |
| | LQMNSLRAED | LTISSLQPE | | | | | | |
| | TAVYYCARDR | DFATYYCQQ | | | | | | |
| | PYYYWMDVWG | YKSYPVTFG | | | | | | |
| | KGTTVTVSS | QGTKVEIK | | | | | | |
| | (SEQ ID | (SEQ ID | | | | | | |
| | NO: 1388) | NO: 592) | | | | | | |
| MIAB171 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PSYYY | VSRSL | (SEQ | YPVT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 1433) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PSYYYMDVWG | YKSYPVTFG | | | | | | |
| | KGTTVTVSS | QGTKVEIK | | | | | | |
| | (SEQ ID | (SEQ ID | | | | | | |
| | NO: 1389) | NO: 592) | | | | | | |
| MIAB172 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PTYYY | VSRSL | (SEQ | YPVT |
| | SCAASGFTES | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 1434) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PTYYYMDVWG | YKSYPVTFG | | | | | | |
| | KGTTVTVSS | QGTKVEIK | | | | | | |
| | (SEQ ID | (SEQ ID | | | | | | |
| | NO: 1390) | NO: 592) | | | | | | |
| MIAB173 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYY | VSRSL | (SEQ | YPVT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 360) | 361) | | |
| | SRDNSKNTLY | GSGSGTDET | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 591) | NO: 592) | | | | | | |
| MIAB174 | EVQLLESGGG | DIQMTQSPS | FTFSD | SYISG | CARDR | RASQS | SSLQS | QQYKS |
| | LVQPGGSLRL | SLSASVGDR | YWMH | DSGYT | PYYYY | VSRSL | (SEQ | YPVT |
| | SCAASGFTFS | VTITCRASQ | (SEQ | NYA | MDVW | A | ID | (SEQ |
| | DYWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 1435) | NO: | NO: | NO: | | 363) |
| | ADSVKGRFTI | QSGVPSRES | | 170) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1391) | NO: 592) | | | | | | |
| MIAB175 | EVQLLESGGG | DIQMTQSPS | FTFSS | SYISG | CARDR | RASQS | SSLQS | QQYKS |
| | LVQPGGSLRL | SLSASVGDR | YWMH | DSGYT | PYYYY | VSRSL | (SEQ | YPVT |
| | SCAASGFTFS | VTITCRASQ | (SEQ | NYA | MDVW | A | ID | (SEQ |
| | SYWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 83) | NO: | NO: | NO: | | 363) |
| | ADSVKGRFTI | QSGVPSRFS | | 170) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1392) | NO: 592) | | | | | | |
| MIAB176 | EVQLLESGGG | DIQMTQSPS | FTFSD | SAISG | CARDR | RASQS | SSLQS | QQYKS |
| | LVQPGGSLRL | SLSASVGDR | FWMH | SSGST | PYYYY | VSRSL | (SEQ | YPVT |
| | SCAASGFTES | VTITCRASQ | (SEQ | YYA | MDVW | A | ID | (SEQ |
| | DFWMHWVRQA | SVSRSLAWY | ID | (SEQ | (SEQ | (SEQ | NO: | ID |
| | PGKGLEWISA | QQKPGKAPK | NO: | ID | ID | ID | 362) | NO: |
| | ISGSSGSTYY | LLIYAASSL | 359) | NO: | NO: | NO: | | 363) |
| | ADSVKGRFTI | QSGVPSRES | | 1436) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1393) | NO: 592) | | | | | | |
| MIAB177 | EVQLLESGGG | DIQMTQSPS | FTFSD | SAISG | CARDR | RASQS | SSLQS | QQYKS |
| | LVQPGGSLRL | SLSASVGDR | FWMH | SGGST | PYYYY | VSRSL | (SEQ | YPVT |
| | SCAASGFTFS | VTITCRASQ | (SEQ | YYA | MDVW | A | ID | (SEQ |
| | DFWMHWVRQA | SVSRSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 359) | NO: | NO: | NO: | | 363) |
| | ADSVKGRFTI | QSGVPSRES | | 103) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1357) | NO: 592) | | | | | | |
| MIAB178 | EVQLLESGGG | DIQMTQSPS | FTFSS | SAISG | CARDR | RASQS | SSLQS | QQYKS |
| | LVQPGGSLRL | SLSASVGDR | YWMH | SSGST | PYYYY | VSRSL | (SEQ | YPVT |
| | SCAASGFTFS | VTITCRASQ | (SEQ | YYA | MDVW | A | ID | (SEQ |
| | SYWMHWVRQA | SVSRSLAWY | ID | (SEQ | (SEQ | (SEQ | NO: | ID |
| | PGKGLEWISA | QQKPGKAPK | NO: | ID | ID | ID | 362) | NO: |
| | ISGSSGSTYY | LLIYAASSL | 83) | NO: | NO: | NO: | | 363) |
| | ADSVKGRFTI | QSGVPSRES | | 1436) | 360) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1394) | NO: 592) | | | | | | |
| MIAB179 | EVQLLESGGG | DIQMTQSPS | FTFSS | SAISG | CARDR | RASQS | SSLQS | QQYKS |
| | LVQPGGSLRL | SLSASVGDR | YWMH | SGGST | PYYYY | ISRYL | (SEQ | YPVT |
| | SCAASGFTFS | VTITCRASQ | (SEQ | YYA | MDVW | A | ID | (SEQ |
| | SYWMHWVRQA | SISRYLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 83) | NO: | NO: | NO: | | 363) |
| | ADSVKGRFTI | QSGVPSRES | | 103) | 360) | 1413) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1395) | NO: 1368) | | | | | | |
| MIAB180 | EVQLLESGGG | DIQMTQSPS | FTFSS | SAISG | CARDR | RASQS | SSLQS | QQYKS |
| | LVQPGGSLRL | SLSASVGDR | YWMH | SGGST | PYYYY | VSRSL | (SEQ | YPVT |
| | SCAASGFTES | VTITCRASQ | (SEQ | YYA | MDVW | A | ID | (SEQ |
| | SYWMHWVRQA | SVSRSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 83) | NO: | NO: | NO: | | 363) |
| | ADSVKGRFTI | QSGVPSRFS | | 103) | 360) | 361) | | |
| | SRDNSKNTLY | GSGSGTDFT | | | | | | |
| | LQMNSLRAED | LTISSLQPE | | | | | | |
| | TAVYYCARDR | DFATYYCOQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1395) | NO: 592) | | | | | | |
| MIAB181 | EVQLLESGGG | DIQMTQSPS | FTFSS | SAISG | CARDR | RASQS | SSLQS | QQYKS |
| | LVQPGGSLRL | SLSASVGDR | YWMH | SGGST | PYYYY | ISRYL | (SEQ | YPVT |
| | SCAASGFTFS | VTITCRASQ | (SEQ | YYA | MDVW | A | ID | (SEQ |
| | SYWMHWVRQA | SISRYLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 83) | NO: | NO: | NO: | | 363) |
| | ADSVKGRFTI | QSGVPSRES | | 103) | 360) | 1413) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1395) | NO: 1368) | | | | | | |

| | | | | | | | | |
|---|---|---|---|---|---|---|---|---|
| MIAB182 | EVQLLESGGG | DIQMTQSPS | FTFSS | SAISG | CARDR | RASQS | SSLQS | QQYKS |
| | LVQPGGSLRL | SLSASVGDR | YWMH | SGGST | PYYYY | ISRSL | (SEQ | YPVT |
| | SCAASGFTFS | VTITCRASQ | (SEQ | YYA | MDVW | A | ID | (SEQ |
| | SYWMHWVRQA | SISRSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 83) | NO: | NO: | NO: | | 363) |
| | ADSVKGRFTI | QSGVPSRES | | 103) | 360) | 1408) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1395) | NO: 1363) | | | | | | |
| MIAB183 | EVQLLESGGG | DIQMTQSPS | FTFSS | SAISG | CARDR | RASQS | SSLQS | QQYKS |
| | LVQPGGSLRL | SLSASVGDR | YWMH | SGGST | PYYYY | VSSSL | (SEQ | YPVT |
| | SCAASGFTFS | VTITCRASQ | (SEQ | YYA | MDVW | A | ID | (SEQ |
| | SYWMHWVRQA | SVSSSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 83) | NO: | NO: | NO: | | 363) |
| | ADSVKGRFTI | QSGVPSRFS | | 103) | 360) | 1409) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1395) | NO: 1364) | | | | | | |
| MIAB184 | EVQLLESGGG | DIQMTQSPS | FTFSS | SAISG | CARDR | RASQS | SSLQS | QQYKS |
| | LVQPGGSLRL | SLSASVGDR | YWMH | SGGST | PYYYY | ISSSL | (SEQ | YPVT |
| | SCAASGFTES | VTITCRASQ | (SEQ | YYA | MDVW | A | ID | (SEQ |
| | SYWMHWVRQA | SISSSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 83) | NO: | NO: | NO: | | 363) |
| | ADSVKGRFTI | QSGVPSRES | | 103) | 360) | 1412) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYYMDVWG | YKSYPVTFG | | | | | | |
| | KGTTVTVSS | QGTKVEIK | | | | | | |
| | (SEQ ID | (SEQ ID | | | | | | |
| | NO: 1395) | NO: 1367) | | | | | | |
| MIAB185 | EVQLLESGGG | DIQMTQSPS | FTFSS | SYISG | CARDR | RASQS | SSLQS | QQYKS |
| | LVQPGGSLRL | SLSASVGDR | YWMH | DSGYT | PYYYI | ISRSL | (SEQ | YPVT |
| | SCAASGFTFS | VTITCRASQ | (SEQ | NYA | MDVW | A | ID | (SEQ |
| | SYWMHWVRQA | SISRSLAWY | ID | (SEQ | SEQ | (SEQ | NO: | ID |
| | PGKGLEWISY | QQKPGKAPK | NO: | ID | ID | ID | 362) | NO: |
| | ISGDSGYTNY | LLIYAASSL | 83) | NO: | NO: | NO: | | 363) |
| | ADSVKGRFTI | QSGVPSRFS | | 170) | 1431) | 1408) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1396) | NO: 1363) | | | | | | |
| MIAB186 | EVQLLESGGG | DIQMTQSPS | FTFSS | SYISG | CARDR | RASQS | SSLQS | QQYKS |
| | LVQPGGSLRL | SLSASVGDR | YWMH | DSGYT | PYYYI | VSSSL | (SEQ | YPVT |
| | SCAASGFTFS | VTITCRASQ | (SEQ | NYA | MDVW | A | ID | (SEQ |
| | SYWMHWVRQA | SVSSSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 83) | NO: | NO: | NO: | | 363) |
| | ADSVKGRFTI | QSGVPSRFS | | 170) | 1431) | 1409) | | |
| | SRDNSKNTLY | GSGSGTDFT | | | | | | |
| | LQMNSLRAED | LTISSLQPE | | | | | | |
| | TAVYYCARDR | DFATYYCOQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1396) | NO: 1364) | | | | | | |
| MIAB187 | EVQLLESGGG | DIQMTQSPS | FTFSS | SYISG | CARDR | RASQS | SSLQS | QQYKS |
| | LVQPGGSLRL | SLSASVGDR | YWMH | DSGYT | PYYYI | ISSSL | (SEQ | YPVT |
| | SCAASGFTFS | VTITCRASQ | (SEQ | NYA | MDVW | A | ID | (SEQ |
| | SYWMHWVRQA | SISSSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 83) | NO: | NO: | NO: | | 363) |
| | ADSVKGRFTI | QSGVPSRES | | 170) | 1431) | 1412) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1396) | NO: 1367) | | | | | | |
| MIAB188 | EVQLLESGGG | DIQMTQSPS | FTFSD | SAISG | CARDR | RASQS | SSLQS | QQYKS |
| | LVQPGGSLRL | SLSASVGDR | FWMH | SGGST | PYYYI | ISRSL | (SEQ | YPVT |
| | SCAASGFTES | VTITCRASQ | (SEQ | YYA | MDVW | A | ID | (SEQ |
| | DFWMHWVRQA | SISRSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 359) | NO: | NO: | NO: | | 363) |
| | ADSVKGRFTI | QSGVPSRFS | | 103) | 1431) | 1408) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1397) | NO: 1363) | | | | | | |
| MIAB189 | EVQLLESGGG | DIQMTQSPS | FTFSD | SAISG | CARDR | RASQS | SSLQS | QQYKS |
| | LVQPGGSLRL | SLSASVGDR | FWMH | SGGST | PYYYI | VSSSL | (SEQ | YPVT |
| | SCAASGFTES | VTITCRASQ | (SEQ | YYA | MDVW | A | ID | (SEQ |
| | DFWMHWVRQA | SVSSSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 359) | NO: | NO: | NO: | | 363) |
| | ADSVKGRFTI | QSGVPSRES | | 103) | 1431) | 1409) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1397) | NO: 1364) | | | | | | |
| MIAB190 | EVQLLESGGG | DIQMTQSPS | FTFSD | SAISG | CARDR | RASQS | SSLQS | QQYKS |
| | LVQPGGSLRL | SLSASVGDR | FWMH | SGGST | PYYYI | ISSSL | (SEQ | YPVT |
| | SCAASGFTFS | VTITCRASQ | (SEQ | YYA | MDVW | A | ID | (SEQ |
| | DFWMHWVRQA | SISSSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 359) | NO: | NO: | NO: | | 363) |
| | ADSVKGRFTI | QSGVPSRFS | | 103) | 1431) | 1412) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1397) | NO: 1367) | | | | | | |
| MIAB191 | EVQLLESGGG | DIQMTQSPS | FTFSS | SAISG | CARDR | RASQS | SSLQS | QQYKS |
| | LVQPGGSLRL | SLSASVGDR | YWMH | SGGST | PYYYI | ISRSL | (SEQ | YPVT |
| | SCAASGFTFS | VTITCRASQ | (SEQ | YYA | MDVW | A | ID | (SEQ |
| | SYWMHWVRQA | SISRSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 83) | NO: | NO: | NO: | | 363) |
| | ADSVKGRFTI | QSGVPSRFS | | 103) | 1431) | 1408) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1398) | NO: 1363) | | | | | | |
| MIAB192 | EVQLLESGGG | DIQMTQSPS | FTFSS | SAISG | CARDR | RASQS | SSLQS | QQYKS |
| | LVQPGGSLRL | SLSASVGDR | YWMH | SGGST | PYYYI | VSSSL | (SEQ | YPVT |
| | SCAASGFTFS | VTITCRASQ | (SEQ | YYA | MDVW | A | ID | (SEQ |
| | SYWMHWVRQA | SVSSSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 83) | NO: | NO: | NO: | | 363) |
| | ADSVKGRFTI | QSGVPSRES | | 103) | 1431) | 1409) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1398) | NO: 1364) | | | | | | |
| MIAB193 | EVQLLESGGG | DIQMTQSPS | FTFSS | SAISG | CARDR | RASQS | SSLQS | QQYKS |
| | LVQPGGSLRL | SLSASVGDR | YWMH | SGGST | PYYYI | ISSSL | (SEQ | YPVT |
| | SCAASGFTFS | VTITCRASQ | (SEQ | YYA | MDVW | A | ID | (SEQ |
| | SYWMHWVRQA | SISSSLAWY | ID | (SEQ | (SEQ | (SEQ | NO: | ID |
| | PGKGLEWVSA | QQKPGKAPK | NO: | ID | ID | ID | 362) | NO: |
| | ISGSGGSTYY | LLIYAASSL | 83) | NO: | NO: | NO: | | 363) |
| | ADSVKGRFTI | QSGVPSRES | | 103) | 1431) | 1412) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1398) | NO: 1367) | | | | | | |
| MIAB194 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYI | ISRSL | (SEQ | YPVT |
| | SCAASGFTFS | VTITCRASQ | (SEQ | NYA | MDVW | A | ID | (SEQ |
| | DFWMHWVRQA | SISRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRFS | | 170) | 1431) | 1408) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1387) | NO: 1363) | | | | | | |
| MIAB195 | EVQLLESGGG | DIQMTQSPS | FTFSI | SYISG | CARDR | RASQS | SSLQS | QQYKS |
| | LVQPGGSLRL | SLSASVGDR | FWMH | DSGYT | PYYYI | VSSSL | (SEQ | YPVT |
| | SCAASGFTFS | VTITCRASQ | (SEQ | NYA | MDVW | A | ID | (SEQ |
| | DFWMHWVRQA | SVSSSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 1431) | 1409) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1387) | NO: 1364) | | | | | | |
| MIAB196 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYI | ISSSL | (SEQ | YPVT |
| | SCAASGFTES | VTITCRASQ | (SEQ | NYA | MDVW | A | ID | (SEQ |
| | DFWMHWVRQA | SISSSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 1431) | 1412) | | |
| | SRDNSKNTLY | GSGSGTDET | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1387) | NO: 1367) | | | | | | |
| MIAB197 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYI | ISRSL | (SEQ | YPVT |
| | SCAASGFTES | VTITCRASQ | (SEQ | NYA | MDVW | A | ID | (SEQ |
| | DFWMHWVRQA | SISRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 1431) | 1408) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1387) | NO: 1363) | | | | | | |
| MIAB198 | EVQLLESGGG | DIQMTQSPS | FTFSD | SYISG | CARDR | RASQS | SSLQS | QQYKS |
| | LVQPGGSLRL | SLSASVGDR | FWMH | DSGYT | PYYYI | VSRSL | (SEQ | YPVT |
| | SCAASGFTFS | VTITCRASQ | (SEQ | NYA | MDVW | A | ID | (SEQ |
| | DFWMHWVRQA | SVSRSLAWY | ID | (SEQ | (SEQ | (SEQ | NO: | ID |
| | PGKGLEWISY | QQKPGKAPK | NO: | ID | ID | ID | 362) | NO: |
| | ISGDSGYTNY | LLIYAASSL | 359) | NO: | NO: | NO: | | 363) |
| | ADSVKGRFTI | QSGVPSRES | | 170) | 1431) | 361) | | |
| | SRDNSKNTLY | GSGSGTDFT | | | | | | |
| | LQMNSLRAED | LTISSLQPE | | | | | | |
| | TAVYYCARDR | DFATYYCQQ | | | | | | |
| | PYYYIMDVWG | YKSYPVTFG | | | | | | |
| | KGTTVTVSS | QGTKVEIK | | | | | | |
| | (SEQ ID | (SEQ ID | | | | | | |
| | NO: 1387) | NO: 592) | | | | | | |

In some embodiments, the MAdCAM antibody comprises one or more sequences, or a combination thereof, of the sequences presented in Table 9.

In some embodiments, the antibody is linked to another antibody or therapeutic. In some embodiments, the MAdCAM antibody is linked to a PD-1 antibody or an IL-2 mutein as provided herein or that is incorporated by reference.

In some embodiments, the variable light chain MAdCAM antibody comprises a mutation selected from the group comprising V29I; R31S; S32Y; A34N; Y91S; K92Y; Y94T; and V99R.

In some embodiments, the variable heavy chain MAdCAM antibody comprises a mutation selected from the group comprising D31S, F32Y, I48V, Y50A, D54S, Y57S, N59Y, Y103G, V29I, R31S; D31S, F32Y, I48V, Y50A, D54S, Y57S, N59Y, V29I, R31S; D31S, F32Y, I48V, Y50A, D54S, Y57S, N59Y, Y103G, V29I; D31S, F32Y, I48V, Y50A, D54S, Y57S, N59Y, V29I; D31S, F32Y, Y50A, D54S, S55G, Y57S, N59Y, Y103G, V29I, R31S; D31S, F32Y, Y50A, D54S, S55G, Y57S, N59Y, V29I, R31S; D31S, F32Y, Y50A, D54S, S55G, Y57S, N59Y, Y103G, V29I; D31S, F32Y, Y50A, D54S, S55G, Y57S, N59Y, V29I; D31S, F32Y, I48V, Y50A, D54S, S55G, Y57S, N59Y, Y103G, V29I, R31S; D31S, F32Y, I48V, Y50A, D54S, S55G, Y57S, N59Y, V29I, R31S; D31S, F32Y, I48V, Y50A, D54S, S55G, Y57S, N59Y, Y103G, V29I; D31S, F32Y, I48V, Y50A, D54S, S55G, Y57S, N59Y, V29I; D31S, F32Y, I48V, D54S, S55G, Y103G, V29I, R31S; D31S, F32Y, I48V, Y50A, D54S, S55G, Y57S, N59Y, Y105D, V29I, R31S; D31S, F32Y, I48V, D54S, S55G, Y105D, V29I, R31S; D31S, F32Y, I48V, Y50A, D54S, S55G, Y57S, N59Y, Y103G, V29I, R31S; D31S, F32Y, I48V, D54S, S55G, Y105D, V29I, R31S; D31S; F32Y; W33A; H35S; I48V; Y50A; D54S; S55G; Y57S; N59Y; D60A; D60Q; N72A; N72Q; N82A; N82G; and N82Q.

In some embodiments, the MAdCAM antibody comprises one or more sequences as shown in Table 6 or Table 9. In some embodiments, the MAdCAM antibody comprises a combination of one or more sequence as shown in Table 6, or Table 9. In some embodiments, the MAdCAM antibody is in a scFV format as illustrated in Table 6. In some embodiments, the antibody comprises a CDR1 from any one of clones 1-66 of Table 6, a CDR2 from any one of clones 1-84, and a CDR3 from any one of clones 1-66 of Table 6. In some embodiments, the antibody comprises a LCDR1 from any one of clones 1-66 of Table 6, a LCDR2 from any one of clones 1-66 of Table 6, and a LCDR3 from any one of clones 1-66 of Table 6. In some embodiments, the MAdCAM antibody is in a Fab format as illustrated in Table 9. In some embodiments, the antibody comprises a HCDR1 from any one of clones MIAB128-198 of Table 9, a HCDR2 from any one of clones MIAB128-198 of Table 9, and a HCDR3 from any one of clones MIAB128-198 of Table 9. In some embodiments, the antibody comprises a LCDR1 from any one of clones MIAB128-198 of Table 9, a LCDR2 from any one of clones MIAB128-198 of Table 9, and a LCDR3 from any one of clones MIAB128-198 of Table 9. In some embodiments, the amino acid residues of the CDRs shown above contain mutations. In some embodiments, the CDRs contain 0, 1, 2, 3, 4, 5, 6, 7, 8, 9, or 10 substitutions or mutations. In some embodiments, the substitution is a conservative substitution.

In some embodiments, the MAdCAM antibody has a VH region selected from any one of clones 1-84 of Table 7 and a VL region selected from any one of clones 1-84 as set forth in of Table 7. In some embodiments, the antibody comprises a CDR1 from any one of clones 1-84 of Table 7, a CDR2 from any one of clones 1-84, and a CDR3 from any one of clones 1-84 of Table 7. In some embodiments, the antibody comprises a LCDR1 from any one of clones 1-84 of Table 7, a LCDR2 from any one of clones 1-84 of Table 7, and a LCDR3 from any one of clones 1-84 of Table 7. In some embodiments, the MAdCAM antibody has a VH region selected from any one of clones MIAB128-198 of Table 9 and a VK region selected from any one of clones MIAB128-198 as set forth in of Table 9. In some embodiments, the antibody comprises a CDR1 from any one of clones MIAB128-198 of Table 9, a CDR2 from any one of clones MIAB128-198, and a CDR3 from any one of clones MIAB128-198 of Table 9. In some embodiments, the antibody comprises a LCDR1 from any one of clones MIAB128-198 of Table 9, a LCDR2 from any one of clones MIAB128-198 of Table 9, and a LCDR3 from any one of clones MIAB128-198 of Table 9.

In some embodiments, the amino acid residues of the CDRs shown above contain mutations. In some embodiments, the CDRs contain 0, 1, 2, 3, 4, 5, 6, 7, 8, 9, or 10 substitutions or mutations. In some embodiments, the substitution is a conservative substitution.

In some embodiments, the molecule comprises an antibody that binds to MAdCAM. In some embodiments, the antibody comprises (i) a heavy chain variable region comprising heavy chain CDR1, CDR2, and CDR3 sequences, wherein the heavy chain CDR1 sequence has the amino acid sequence of any of the CDR1 sequences set forth in Table 6, Table 7, or Table 9; the heavy chain CDR2 has the amino acid sequence of any of the CDR2 sequences set forth in Table 6, Table 7, or Table 9, and the heavy chain CDR3 has the amino acid sequence of any of the CDR3 sequences set forth in Table 6, Table 7, or Table 9; or variants of any of the foregoing; and (ii) a light chain variable region comprising light chain CDR1, CDR2, and CDR3 sequences, wherein the light chain CDR1 sequence has the amino acid sequence of any of the LCDR1 sequences set forth in Table 6, Table 7, or Table 9; the light chain LCDR2 has the amino acid sequence of any of the LCDR2 sequences set forth in Table 6, Table 7, or Table 9, and the light chain CDR3 has the amino acid sequence of any of the LCDR3 sequences set forth in Table 6, Table 7, or Table 9, or variants of any of the foregoing.

In some embodiments, the antibody comprises a heavy chain variable region comprising heavy chain CDR1, CDR2, and CDR3 sequences, wherein the heavy chain CDR1, CDR2, and CDR3 sequences have the amino acid sequence as set forth in Antibody 6 of Table 6 or Antibody 6 of Table 7, or variants of any of the foregoing; and (ii) a light chain variable region comprising light chain CDR1, CDR2, and CDR3 sequences, wherein the light chain CDR1, CDR2, and CDR3 sequences have the amino acid sequence as set forth sequence as set forth in Antibody 6 of Table 6 or Antibody 6 of Table 7, or variants of any of the foregoing.

In some embodiments, the antibody comprises a heavy chain variable region comprising heavy chain CDR1, CDR2, and CDR3 sequences, wherein the heavy chain CDR1, CDR2, and CDR3 sequences have the amino acid sequence as set forth in Antibody 59 of Table 6 or Antibody 75 of Table 7, or variants of any of the foregoing; and (ii) a light chain variable region comprising light chain CDR1, CDR2, and CDR3 sequences, wherein the light chain CDR1, CDR2, and CDR3 sequences have the amino acid sequence as set forth sequence as set forth in Antibody 59 of Table 6 or Antibody 75 of Table 7, or variants of any of the foregoing.

In some embodiments, the antibody comprises a heavy chain variable region comprising heavy chain CDR1, CDR2, and CDR3 sequences, wherein the heavy chain CDR1, CDR2, and CDR3 sequences have the amino acid sequence as set forth in Antibody 63 of Table 6 or Antibody 79 of Table 7, or variants of any of the foregoing; and (ii) a light chain variable region comprising light chain CDR1, CDR2, and CDR3 sequences, wherein the light chain CDR1, CDR2, and CDR3 sequences have the amino acid sequence as set forth sequence as set forth in Antibody 63 of Table 6 or Antibody 79 of Table 7, or variants of any of the foregoing.

In some embodiments, the antibody comprises a heavy chain variable region comprising heavy chain CDR1, CDR2, and CDR3 sequences, wherein the heavy chain CDR1, CDR2, and CDR3 sequences have the amino acid sequence as set forth in MIAB197 of Table 9, or variants of any of the foregoing; and (ii) a light chain variable region comprising light chain CDR1, CDR2, and CDR3 sequences, wherein the light chain CDR1, CDR2, and CDR3 sequences have the amino acid sequence as set forth sequence as set forth in MIAB197 of Table 9, or variants of any of the foregoing.

These are non-limiting illustrative examples and the antibodies can have the CDRs as set forth in the tables provided herein and are explicitly referenced without writing out the previous paragraphs for each CDR set.

In some embodiments, the MAdCAM antibody comprises a VH and VL(VK) chain as provided herein, such as those listed in the Table 7, MAdCAM Antibody CDR Table 1, and Table 9. In some embodiments, the VH peptide comprises a sequence of SEQ ID NO: 414, 59I, 599, or 1387. In some embodiments, the VK chain comprises a sequence of 415, 592, 600, or 1363. In some embodiments, the antibody comprises a VH of SEQ ID NO: 414 and a VK of SEQ ID NO: 415. In some embodiments, the antibody comprises a VH of SEQ ID NO: 591 and a VK of SEQ ID NO: 592. In some embodiments, the antibody comprises a VH of SEQ ID NO: 599 and a VK of SEQ ID NO: 600. In some embodiments, the antibody comprises a VH of SEQ ID NO: 1387 and a VK of SEQ ID NO: 1363. The VH and VK can also be in a scFV format as illustrated in the Table 6, Table 11, Table 12, and Table 14. The VH and VK can also be in a Fab format as illustrated in the Table 9.

In some embodiments, a therapeutic is provided comprising one or more of the following polypeptides:

| SEQ ID NO: | Sequence |
|---|---|
| 620 | EVQLLESGGGLVQPGGSLRLSCAASGFTFNNYAFHWVRQAPGKGLEWVSRINSYGTSTTYADSVKGRF |
| | TISRDNSKNTLYLQMNSLRAEDTAVYYCAREGPVAGYWYFDLWGQGTLVTVSSASTKGPSVFPLAPSS |
| | KSTSGGTAALGCLVKDYFPEPVTVSWNSGALTSGVHTFPAVLQSSGLYSLSSVVTVPSSSLGTQTYIC |
| | NVNHKPSNTKVDKKVEPKSCDKTHTCPPCPAPEAAGAPSVFLFPPKPKDTLMISRTPEVTCVVVDVSH |
| | EDPEVKFNWYVDGVEVHNAKTKPREEQYNSTYRVVSVLTVLHQDWLNGKEYKCKVSNKALPAPIEKTI |
| | SKAKGQPREPQVYTLPPSREEMTKNQVSLTCLVKGFYPSDIAVEWESNGQPENNYKTTPPVLDSDGSF |
| | FLYSKLTVDKSRWQQGNVFSCSVMHEALHNHYTQKSLSLSPGGGGGSAPTSSSTKKTQLQLEHLLLDL |
| | QMILNGINNYKNPKLTRMLTFKFYMPKKATELKHLQCLEEELKPLEEALNLAPSKNFHLRPRDLISDI |
| | NVIVLELKGSETTFMCEYADETATIVEFINRWITFSQSIISTLT |
| 621 | DIQMTQSPSSLSASVGDRVTITCRASQIIGTNLAWYQQKPGKAPKLLIYGASSLQSGVPSRFSGSGSG |
| | TDFTLTISSLQPEDFATYYCQQSYRLPFTFGQGTKVEIKRRTVAAPSVFIFPPSDEQLKSGTASVVCL |
| | LNNFYPREAKVOWKVDNALQSGNSQESVTEQDSKDSTYSLSSTLTLSKADYEKHKVYACEVTHQGLSS |
| | PVTKSENRGEC |
| 622 | EVQLLESGGGLVQPGGSLRLSCAASGFTFSDFWMHWVRQAPGKGLEWISYISGDSGYTNYADSVKGRF |
| | TISRDNSKNTLYLQMNSLRAEDTAVYYCARDRPYYYYMDVWGKGTTVTVSSASTKGPSVFPLAPSSKS |
| | TSGGTAALGCLVKDYFPEPVTVSWNSGALTSGVHTFPAVLQSSGLYSLSSVVTVPSSSLGTQTYICNV |
| | NHKPSNTKVDKKVEPKSCDKTHTCPPCPAPEAAGAPSVFLFPPKPKDTLMISRTPEVTCVVVDVSHED |
| | PEVKFNWYVDGVEVHNAKTKPREEQYNSTYRVVSVLTVLHQDWLNGKEYKCKVSNKALPAPIEKTISK |
| | AKGQPREPQVYTLPPSREEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPGGGGGSAPTSSSTKKTQLQLEHLLLDLQM |
| | ILNGINNYKNPKLTRMLTFKFYMPKKATELKHLQCLEEELKPLEEALNLAPSKNFHLRPRDLISDINV |
| | IVLELKGSETTFMCEYADETATIVEFINRWITFSQSIISTLT |
| 623 | DIQMTQSPSSLSASVGDRVTITCRASQSVSRSLAWYQQKPGKAPKLLIYAASSLQSGVPSRFSGSGSG |
| | TDFTLTISSLQPEDFATYYCQQYKSYPVTFGQGTKVEIKRTVAAPSVFIFPPSDEQLKSGTASVVCLL |
| | NNFYPREAKVQWKVDNALQSGNSQESVTEQDSKDSTYSLSSTLTLSKADYEKHKVYACEVTHQGLSSP |
| | VTKSENRGEC |
| 624 | EVQLLESGGGLVKPGGSLRLSCAASGFTFDDYAMHWVRQAPGKGLEWVASITSSSAFIDYAASVKGRF |
| | TISRDDSKNTLYLQMNSLKTEDTAVYYCARERVDWNSYFDLWGRGTLVTVSSASTKGPSVFPLAPSSK |
| | STSGGTAALGCLVKDYFPEPVTVSWNSGALTSGVHTFPAVLQSSGLYSLSSVVTVPSSSLGTQTYICN |
| | VNHKPSNTKVDKKVEPKSCDKTHTCPPCPAPEAAGAPSVELFPPKPKDTLMISRTPEVTCVVVDVSHE |
| | DPEVKFNWYVDGVEVHNAKTKPREEQYNSTYRVVSVLTVLHQDWLNGKEYKCKVSNKALPAPIEKTIS |
| | KAKGQPREPQVYTLPPSREEMTKNQVSLTCLVKGFYPSDIAVEWESNGQPENNYKTTPPVLDSDGSFF |
| | LYSKLTVDKSRWQQGNVFSCSVMHEALHNHYTQKSLSLSPGGGGGSAPTSSSTKKTQLQLEHLLLDLQ |
| | MILNGINNYKNPKLTRMLTFKFYMPKKATELKHLQCLEEELKPLEEALNLAPSKNFHLRPRDLISDIN |
| | VIVLELKGSETTFMCEYADETATIVEFINRWITFSQSIISTLT |
| 625 | EIVMTQSPATLSVSPGERATLSCRASQGISNSYLAWYQQKPGQAPRLLIYGASTRATGIPARFSGSGS |
| | GTEFTLTISSLQSEDFAVYYCQQYYTYPPTFGPGTKVDIKRTVAAPSVFIFPPSDEQLKSGTASVVCL |
| | LNNFYPREAKVQWKVDNALQSGNSQESVTEQDSKDSTYSLSSTLTLSKADYEKHKVYACEVTHQGLSS |
| | PVTKSENRGEC |

In some embodiments, the polypeptide comprises one peptide of SEQ ID NO: 620, 622, or 624 and a second peptide of SEQ ID NO: 621, 623, or 625. In some embodiments, a polypeptide is provided comprising a first peptide of SEQ ID NO: 620 and a second peptide comprising a sequence of SEQ ID NO: 621. In some embodiments, a polypeptide is provided comprising a first peptide of SEQ ID NO: 620 and a second peptide comprising a sequence of SEQ ID NO: 623. In some embodiments, a polypeptide is provided comprising a first peptide of SEQ ID NO: 620 and a second peptide comprising a sequence of SEQ ID NO: 625. In some embodiments, a polypeptide is provided comprising a first peptide of SEQ ID NO: 622 and a second peptide comprising a sequence of SEQ ID NO: 621. In some embodiments, a polypeptide is provided comprising a first peptide of SEQ ID NO: 622 and a second peptide comprising a sequence of SEQ ID NO: 623. In some embodiments, a polypeptide is provided comprising a first peptide of SEQ ID NO: 622 and a second peptide comprising a sequence of SEQ ID NO: 625. In some embodiments, a polypeptide is provided comprising a first peptide of SEQ ID NO: 624 and a second peptide comprising a sequence of SEQ ID NO: 621. In some embodiments, a polypeptide is provided comprising a first peptide of SEQ ID NO: 624 and a second peptide comprising a sequence of SEQ ID NO: 623. In some embodiments, a polypeptide is provided comprising a first peptide of SEQ ID NO: 624 and a second peptide comprising a sequence of SEQ ID NO: 625.

In some embodiments, the therapeutic compound comprises a MAdCAM IgG wherein the IL-2 mutein is fused to the C-terminus of the IgG heavy chain, and is selected from one or more of the following sequences:

TABLE 10

| | | | Fc-IL- | | | |
|---|---|---|---|---|---|---|
| | | | 2M | | | |
| | | IgG1 Constant | Linker | | | |
| Ab | VH Seq | Domains Seq | Seq | IL-2M Seq | VK Seq | CK Seq |
| MIAB | EVQLLESG | ASTKGPSVFPLAPSSKSTSG | GGGGS | APTSSSTKKT | DIQMTQS | RTVAAPS |
| 128 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPNYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1346) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 128A | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPQYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1348) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 129 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPGYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1349) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 130 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | QMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1350) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 131 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | GMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1351) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 132 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSNF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | SEQ ID | QQGNVFSCSVMHEALHNHYT | | | IK (SEQ | EC (SEQ |
| | NO: | QKSLSLSPG (SEQ ID | | | ID NO: | ID NO: |
| | 1352) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 133 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1353) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 134 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSAF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1354) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 135 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSNNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1355) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 136 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASQS | TASVVCL |
| | AMSWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1356) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 137 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1357) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 138 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASOS | TASVVCL |
| | AMSWVRQA | YICNVNHKPSNTKVDKKVEP | | TFKFYMPKKA | VSRSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1358) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 139 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISSYLNW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQS | YACEVTH |
| | YMDVWGKG | AVEWESNGQPENNYKTTPPV | | TLT (SEQ | YSTPRTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1360) | 45) |
| MIAB | EVQLLESG | ASTKGPSVFPLAPSSKSTSG | GGGGS | APTSSSTKKT | DIQMTQS | RTVAAPS |
| 140 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASQS | TASVVCL |
| | AMSWVRQA | YICNVNHKPSNTKVDKKVEP | | TFKFYMPKKA | ISSYLNW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQS | YACEVTH |
| | YMDVWGKG | AVEWESNGQPENNYKTTPPV | | TLT (SEQ | YSTPRTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1358) | NO: 44) | | | 1360) | 45) |
| MIAB | EVQLLESG | ASTKGPSVFPLAPSSKSTSG | GGGGS | APTSSSTKKT | DIQMTQS | RTVAAPS |
| 141 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISSYLNW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1361) | 45) |
| MIAB | EVQLLESG | ASTKGPSVFPLAPSSKSTSG | GGGGS | APTSSSTKKT | DIQMTQS | RTVAAPS |
| 142 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQS | YACEVTH |
| | YMDVWGKG | AVEWESNGQPENNYKTTPPV | | TLT (SEQ | YSTPRTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1362) | 45) |
| MIAB | EVQLLESG | ASTKGPSVFPLAPSSKSTSG | GGGGS | APTSSSTKKT | DIQMTQS | RTVAAPS |
| 143 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 592) | 45) |
| MIAB | EVQLLESG | ASTKGPSVFPLAPSSKSTSG | GGGGS | APTSSSTKKT | DIQMTQS | RTVAAPS |
| 144 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 592) | 45) |
| MIAB | EVQLLESG | ASTKGPSVFPLAPSSKSTSG | GGGGS | APTSSSTKKT | DIQMTQS | RTVAAPS |
| 145 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1363) | 45) |
| MIAB | EVQLLESG | ASTKGPSVFPLAPSSKSTSG | GGGGS | APTSSSTKKT | DIQMTQS | RTVAAPS |
| 146 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTESDE | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | VSSSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1364) | 45) |
| MIAB | EVQLLESG | ASTKGPSVFPLAPSSKSTSG | GGGGS | APTSSSTKKT | DIQMTQS | RTVAAPS |
| 147 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRYLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1365) | 45) |
| MIAB | EVQLLESG | ASTKGPSVFPLAPSSKSTSG | GGGGS | APTSSSTKKT | DIQMTQS | RTVAAPS |
| 148 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLNW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1366) | 45) |
| MIAB | EVQLLESG | ASTKGPSVFPLAPSSKSTSG | GGGGS | APTSSSTKKT | DIQMTQS | RTVAAPS |
| 149 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISSSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1367) | 45) |
| MIAB | EVQLLESG | ASTKGPSVFPLAPSSKSTSG | GGGGS | APTSSSTKKT | DIQMTQS | RTVAAPS |
| 150 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | ISRYLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1368) | 45) |
| MIAB | EVQLLESG | ASTKGPSVFPLAPSSKSTSG | GGGGS | APTSSSTKKT | DIQMTQS | RTVAAPS |
| 151 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTOT | 23) | YKNPKLTRML | TCRASOS | TASVVCL |
| | WMHWVRQA | YICNVNHKPSNTKVDKKVEP | | TFKFYMPKKA | ISRSLNW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1369) | 45) |
| MIAB | EVQLLESG | ASTKGPSVFPLAPSSKSTSG | GGGGS | APTSSSTKKT | DIQMTQS | RTVAAPS |
| 152 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSSYLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCOQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1370) | 45) |
| MIAB | EVQLLESG | ASTKGPSVFPLAPSSKSTSG | GGGGS | APTSSSTKKT | DIQMTQS | RTVAAPS |
| 153 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSSSLNW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1371) | 45) |
| MIAB | EVQLLESG | ASTKGPSVFPLAPSSKSTSG | GGGGS | APTSSSTKKT | DIQMTQS | RTVAAPS |
| 154 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRYLNW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 4: | GQGTKVE | TKSFNRG |
| | (SEQ ID | QQGNVFSCSVMHEALHNHYT | | | IK (SEQ | EC (SEQ |
| | NO: 591) | QKSLSLSPG SEQ ID | | | ID NO: | ID NO: |
| | | NO: 44) | | | 1372) | 45) |
| MIAB | EVQLLESG | ASTKGPSVFPLAPSSKSTSG | GGGGS | APTSSSTKKT | DIQMTQS | RTVAAPS |
| 155 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISSSLNW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1373) | 45) |
| MIAB | EVQLLESG | ASTKGPSVFPLAPSSKSTSG | GGGGS | APTSSSTKKT | DIQMTQS | RTVAAPS |
| 156 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | ISRYLNW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1374) | 45) |
| MIAB | EVQLLESG | ASTKGPSVFPLAPSSKSTSG | GGGGS | APTSSSTKKT | DIQMTQS | RTVAAPS |
| 157 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISSYLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1375) | 45) |
| MIAB | EVQLLESG | ASTKGPSVFPLAPSSKSTSG | GGGGS | APTSSSTKKT | DIQMTQS | RTVAAPS |
| 158 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSSYLNW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1376) | 45) |
| MIAB | EVQLLESG | ASTKGPSVFPLAPSSKSTSG | GGGGS | APTSSSTKKT | DIQMTQS | RTVAAPS |
| 159 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGESG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG SEQ ID | | | ID NO: | ID NO: |
| | 1377) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 160 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTQYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1378) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 161 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DQSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1379) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 162 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLGW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1380) | 45) |
| MIAB | EVQLLESG | ASTKGPSVFPLAPSSKSTSG | GGGGS | APTSSSTKKT | DIQMTQS | RTVAAPS |
| 163 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLDW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1381) | 45) |
| MIAB | EVQLLESG | ASTKGPSVFPLAPSSKSTSG | GGGGS | APTSSSTKKT | DIQMTQS | RTVAAPS |
| 164 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | VSRSLTW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1382) | 45) |
| MIAB | EVQLLESG | ASTKGPSVFPLAPSSKSTSG | GGGGS | APTSSSTKKT | DIQMTQS | RTVAAPS |
| 165 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLSW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1383) | 45) |
| MIAB | EVQLLESG | ASTKGPSVFPLAPSSKSTSG | GGGGS | APTSSSTKKT | DIQMTQS | RTVAAPS |
| 166 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | VSRSLEW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1384) | 45) |
| MIAB | EVQLLESG | ASTKGPSVFPLAPSSKSTSG | GGGGS | APTSSSTKKT | DIQMTQS | RTVAAPS |
| 167 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLKW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1385) | 45) |
| MIAB | EVQLLESG | ASTKGPSVFPLAPSSKSTSG | GGGGS | APTSSSTKKT | DIQMTQS | RTVAAPS |
| 168 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLLW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGOPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 1386) | 45) |
| MIAB | EVQLLESG | ASTKGPSVFPLAPSSKSTSG | GGGGS | APTSSSTKKT | DIQMTQS | RTVAAPS |
| 169 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1387) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 170 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | WMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1388) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 171 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPSYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1389) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 172 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPTYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1390) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 173 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 592) | 45) |
| MIAB | EVQLLESG | ASTKGPSVFPLAPSSKSTSG | GGGGS | APTSSSTKKT | DIQMTQS | RTVAAPS |
| 174 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDY | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1391) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 175 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1392) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 176 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SAISGSSG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1393) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 177 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1357) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 178 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SAISGSSG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1394) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 179 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASQS | TASVVCL |
| | WMHWVRQA | YICNVNHKP SNTKVDKKVEP | | TFKFYMPKKA | ISRYLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1395) | NO: 44) | | | 1368) | 45) |
| MIAB | EVQLLESG | ASTKGPSVFPLAPSSKSTSG | GGGGS | APTSSSTKKT | DIQMTQS | RTVAAPS |
| 180 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1395) | NO: 44) | | | 592) | 45) |
| MIAB | EVQLLESG | ASTKGPSVFPLAPSSKSTSG | GGGGS | APTSSSTKKT | DIQMTQS | RTVAAPS |
| 181 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISRYLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKFNW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1395) | NO: 44) | | | 1368) | 45) |
| MIAB | EVQLLESG | ASTKGPSVFPLAPSSKSTSG | GGGGS | APTSSSTKKT | DIQMTQS | RTVAAPS |
| 182 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISRSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1395) | NO: 44) | | | 1363) | 45) |
| MIAB | EVQLLESG | ASTKGPSVFPLAPSSKSTSG | GGGGS | APTSSSTKKT | DIQMTQS | RTVAAPS |
| 183 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSSSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGOPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1395) | NO: 44) | | | 1364) | 45) |
| MIAB | EVQLLESG | ASTKGPSVFPLAPSSKSTSG | GGGGS | APTSSSTKKT | DIQMTQS | RTVAAPS |
| 184 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISSSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSENRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1395) | NO: 44) | | | 1367) | 45) |
| MIAB | EVQLLESG | ASTKGPSVFPLAPSSKSTSG | GGGGS | APTSSSTKKT | DIQMTQS | RTVAAPS |
| 185 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | ISRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1396) | NO: 44) | | | 1363) | 45) |
| MIAB | EVQLLESG | ASTKGPSVFPLAPSSKSTSG | GGGGS | APTSSSTKKT | DIQMTQS | RTVAAPS |
| 186 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSSSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1396) | NO: 44) | | | 1364) | 45) |
| MIAB | EVQLLESG | ASTKGPSVFPLAPSSKSTSG | GGGGS | APTSSSTKKT | DIQMTQS | RTVAAPS |
| 187 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | ISSSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1396) | NO: 44) | | | 1367) | 45) |
| MIAB | EVQLLESG | ASTKGPSVFPLAPSSKSTSG | GGGGS | APTSSSTKKT | DIQMTQS | RTVAAPS |
| 188 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | ISRSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1397) | NO: 44) | | | 1363) | 45) |
| MIAB | EVQLLESG | ASTKGPSVFPLAPSSKSTSG | GGGGS | APTSSSTKKT | DIQMTQS | RTVAAPS |
| 189 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSSSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1397) | NO: 44) | | | 1364) | 45) |
| MIAB | EVQLLESG | ASTKGPSVFPLAPSSKSTSG | GGGGS | APTSSSTKKT | DIOMTQS | RTVAAPS |
| 190 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISSSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1397) | NO: 44) | | | 1367) | 45) |
| MIAB | EVQLLESG | ASTKGPSVFPLAPSSKSTSG | GGGGS | APTSSSTKKT | DIQMTQS | RTVAAPS |
| 191 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISRSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKFNW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1398) | NO: 44) | | | 1363) | 45) |
| MIAB | EVQLLESG | ASTKGPSVFPLAPSSKSTSG | GGGGS | APTSSSTKKT | DIQMTQS | RTVAAPS |
| 192 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSSY | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSSSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1398) | NO: 44) | | | 1364) | 45) |
| MIAB | EVQLLESG | ASTKGPSVFPLAPSSKSTSG | GGGGS | APTSSSTKKT | DIQMTQS | RTVAAPS |
| 193 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTESSY | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISSSLAW | LNNFYPR |
| | PGKGLEWV | KSCDKTHTCPPCPAPEAAGA | | TELKHLQCLE | YQQKPGK | EAKVQWK |
| | SAISGSGG | PSVFLFPPKPKDTLMISRTP | | EELKPLEEAL | APKLLIY | VDNALQS |
| | STYYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1398) | NO: 44) | | | 1367) | 45) |
| MIAB | EVQLLESG | ASTKGPSVFPLAPSSKSTSG | GGGGS | APTSSSTKKT | DIQMTQS | RTVAAPS |
| 194 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | ISRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1387) | NO: 44) | | | 1363) | 45) |
| MIAB | EVQLLESG | ASTKGPSVFPLAPSSKSTSG | GGGGS | APTSSSTKKT | DIQMTQS | RTVAAPS |
| 195 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSSSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1387) | NO: 44) | | | 1364) | 45) |
| MIAB | EVQLLESG | ASTKGPSVFPLAPSSKSTSG | GGGGS | APTSSSTKKT | DIQMTQS | RTVAAPS |
| 196 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | ISSSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1387) | NO: 44) | | | 1367) | 45) |
| MIAB | EVQLLESG | ASTKGPSVFPLAPSSKSTSG | GGGGS | APTSSSTKKT | DIQMTQS | RTVAAPS |
| 197 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | ISRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRES | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1387) | NO: 44) | | | 1363) | 45) |
| MIAB | EVQLLESG | ASTKGPSVFPLAPSSKSTSG | GGGGS | APTSSSTKKT | DIQMTQS | RTVAAPS |
| 198 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASOS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | IMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: | QKSLSLSPG (SEQ ID | | | ID NO: | ID NO: |
| | 1387) | NO: 44) | | | 592) | 45) |
| PRNT | EVQLLESG | ASTKGPSVFPLAPSSKSTSG | GGGGS | APTSSSTKKT | DIQMTQS | RTVAAPS |
| 1 | GGLVQPGG | GTAALGCLVKDYFPEPVTVS | (SEQ | QLQLEHLLLD | PSSLSAS | VFIFPPS |
| | SLRLSCAA | WNSGALTSGVHTFPAVLQSS | ID NO: | LQMILNGINN | VGDRVTI | DEQLKSG |
| | SGFTFSDF | GLYSLSSVVTVPSSSLGTQT | 23) | YKNPKLTRML | TCRASQS | TASVVCL |
| | WMHWVRQA | YICNVNHKPSNTKVDKKVEP | | TFKFYMPKKA | VSRSLAW | LNNFYPR |
| | PGKGLEWI | KSCDKTHTCPPCPAPEAAGA | | TELKHLQCLE | YQQKPGK | EAKVQWK |
| | SYISGDSG | PSVFLFPPKPKDTLMISRTP | | EELKPLEEAL | APKLLIY | VDNALQS |
| | YTNYADSV | EVTCVVVDVSHEDPEVKENW | | NLAPSKNFHL | AASSLQS | GNSQESV |
| | KGRFTISR | YVDGVEVHNAKTKPREEQYN | | RPRDLISDIN | GVPSRFS | TEQDSKD |
| | DNSKNTLY | STYRVVSVLTVLHQDWLNGK | | VIVLELKGSE | GSGSGTD | STYSLSS |
| | LQMNSLRA | EYKCKVSNKALPAPIEKTIS | | TTFMCEYADE | FTLTISS | TLTLSKA |
| | EDTAVYYC | KAKGQPREPQVYTLPPSREE | | TATIVEFINR | LQPEDFA | DYEKHKV |
| | ARDRPYYY | MTKNQVSLTCLVKGFYPSDI | | WITFSQSIIS | TYYCQQY | YACEVTH |
| | YMDVWGKG | AVEWESNGQPENNYKTTPPV | | TLT (SEQ | KSYPVTF | QGLSSPV |
| | TTVTVSS | LDSDGSFFLYSKLTVDKSRW | | ID NO: 41) | GQGTKVE | TKSFNRG |
| | (SEQ ID | QQGNVFSCSVMHEALHNHYT | | | IK (SEQ | EC (SEQ |
| | NO: 591) | QKSLSLSPG (SEQ ID | | | ID NO: | ID NO: |
| | | NO: 44) | | | 592) | 45) |

In some embodiments, the therapeutic compound comprises one or more sequences, or a combination thereof, selected from the Table 10. In some embodiments, the therapeutic compound comprises the peptides of SEQ ID NOs: 1387, 44, 23, 41, 1363, and 45.

In additional embodiments, the MAdCAM antibody comprises an IL-2 mutein fused to the N-terminus of an Fc heavy chain, wherein the Fc is further fused at its C-terminus to a MAdCAM scFv, and has one or more of the sequences as set forth in the following table

TABLE 11

| | | IL-2M- | | | | | |
|---|---|---|---|---|---|---|---|
| | | Fc | | | | Intra | |
| | | Linker | | Fc-scFv | ScFv VH | scFv | ScFv VK |
| Ab | IL-2M Seq | Seq | Fc Domain Seq | Linker | Seq | Linker | Seq |
| MIAB | APTSSSTKK | GGGGSGG | DKTHTCPPCPAPEA | GGGGS | EVQLLESG | GGGGSG | DIQMTQS |
| 199 | TQLQLEHLL | GGSGGGG | AGAPSVFLFPPKPK | (SEQ ID | GGLVQPGG | GGGSGG | PSSLSAS |
| | LDLQMILNG | S (SEQ | DTLMISRTPEVTCV | NO: 23) | SLRLSCAA | GGSGGG | VGDRVTI |
| | INNYKNPKL | ID NO: | VVDVSHEDPEVKFN | | SGFTFSDF | GS | TCRASQS |
| | TRMLTFKFY | 30) | WYVDGVEVHNAKTK | | WMHWVRQA | (SEQ | VSRSLAW |
| | MPKKATELK | | PREEQYNSTYRVVS | | PGKGLEWI | ID NO: | YQQKPGK |
| | HLQCLEEEL | | VLTVLHQDWLNGKE | | SYISGDSG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | YMDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: 591) | | ID NO: |
| | | | 21) | | | | 592) |
| MIAB | APTSSSTKK | GGGGSGG | DKTHTCPPCPAPEA | GGGGSGG | EVQLLESG | GGGGSG | DIQMTQS |
| 200 | TQLQLEHLL | GGSGGGG | AGAPSVFLFPPKPK | GGS | GGLVQPGG | GGGSGG | PSSLSAS |
| | LDLQMILNG | S (SEQ | DTLMISRTPEVTCV | (SEQ ID | SLRLSCAA | GGSGGG | VGDRVTI |
| | INNYKNPKL | ID NO: | VVDVSHEDPEVKFN | NO: | SGFTFSDF | GS | TCRASQS |
| | TRMLTFKFY | 30) | WYVDGVEVHNAKTK | 619) | WMHWVRQA | (SEQ | VSRSLAW |
| | MPKKATELK | | PREEQYNSTYRVVS | | PGKGLEWI | ID NO: | YQQKPGK |
| | HLQCLEEEL | | VLTVLHQDWLNGKE | | SYISGDSG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | YMDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: 591) | | ID NO: |
| | | | 21) | | | | 592) |
| MIAB | APTSSSTKK | GGGGSGG | DKTHTCPPCPAPEA | GGGGSGG | EVQLLESG | GGGGSG | DIQMTQS |
| 201 | TQLQLEHLL | GGSGGGG | AGAPSVFLFPPKPK | GGSGGGG | GGLVQPGG | GGGSGG | PSSLSAS |
| | LDLQMILNG | S (SEQ | DTLMISRTPEVTCV | S (SEQ | SLRLSCAA | GGSGGG | VGDRVTI |
| | INNYKNPKL | ID NO: | VVDVSHEDPEVKFN | ID NO: | SGFTFSDF | GS | TCRASQS |
| | TRMLTFKFY | 30) | WYVDGVEVHNAKTK | 30) | WMHWVRQA | (SEQ | VSRSLAW |
| | MPKKATELK | | PREEQYNSTYRVVS | | PGKGLEWI | ID NO: | YQQKPGK |
| | HLQCLEEEL | | VLTVLHQDWLNGKE | | SYISGDSG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | YMDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: 591) | | ID NO: |
| | | | 21) | | | | 592) |
| MIAB | APTSSSTKK | GGGGSGG | DKTHTCPPCPAPEA | GGGGS | EVQLLESG | GGGGSG | DIQMTQS |
| 202 | TQLQLEHLL | GGSGGGG | AGAPSVFLFPPKPK | (SEQ ID | GGLVQPGG | GGGSGG | PSSLSAS |
| | LDLQMILNG | SGGGGS | DTLMISRTPEVTCV | NO: 23) | SLRLSCAA | GGSGGG | VGDRVTI |
| | INNYKNPKL | (SEQ ID | VVDVSHEDPEVKFN | | SGFTFSDF | GS | TCRASQS |
| | TRMLTFKFY | NO: 22) | WYVDGVEVHNAKTK | | WMHWVRQA | SEQ | VSRSLAW |
| | MPKKATELK | | PREEQYNSTYRVVS | | PGKGLEWI | ID NO: | YQQKPGK |
| | HLQCLEEEL | | VLTVLHQDWLNGKE | | SYISGDSG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | YMDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: 591) | | ID NO: |
| | | | 21) | | | | 592) |
| MIAB | APTSSSTKK | GGGGSGG | DKTHTCPPCPAPEA | GGGGSGG | EVQLLESG | GGGGSG | DIQMTQS |
| 203 | TQLQLEHLL | GGSGGGG | AGAPSVFLFPPKPK | GGS | GGLVQPGG | GGGSGG | PSSLSAS |
| | LDLQMILNG | SGGGGS | DTLMISRTPEVTCV | (SEQ ID | SLRLSCAA | GGSGGG | VGDRVTI |
| | INNYKNPKL | (SEQ ID | VVDVSHEDPEVKFN | NO: | SGFTFSDF | GS | TCRASQS |
| | TRMLTFKFY | NO: 22) | WYVDGVEVHNAKTK | 619) | WMHWVRQA | (SEQ | VSRSLAW |
| | MPKKATELK | | PREEQYNSTYRVVS | | PGKGLEWI | ID NO: | YQQKPGK |
| | HLQCLEEEL | | VLTVLHQDWLNGKE | | SYISGDSG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | YMDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: 591) | | ID NO: |
| | | | 21) | | | | 592) |
| MIAB | APTSSSTKK | GGGGSGG | DKTHTCPPCPAPEA | GGGGSGG | EVQLLESG | GGGGSG | DIQMTQS |
| 204 | TQLQLEHLL | GGSGGGG | AGAPSVFLFPPKPK | GGSGGGG | GGLVQPGG | GGGSGG | PSSLSAS |
| | LDLQMILNG | SGGGGS | DTLMISRTPEVTCV | S (SEQ | SLRLSCAA | GGSGGG | VGDRVTI |
| | INNYKNPKL | (SEQ ID | VVDVSHEDPEVKFN | ID NO: | SGFTFSDF | GS | TCRASQS |
| | TRMLTFKFY | NO: 22) | WYVDGVEVHNAKTK | 30) | WMHWVRQA | (SEQ | VSRSLAW |
| | MPKKATELK | | PREEQYNSTYRVVS | | PGKGLEWI | ID NO: | YQQKPGK |
| | HLQCLEEEL | | VLTVLHQDWLNGKE | | SYISGDSG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | YMDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: 591) | | ID NO: |
| | | | 21) | | | | 592) |
| MIAB | APTSSSTKK | GGGGSGG | DKTHTCPPCPAPEA | GGGGSGG | EVQLLESG | GGGGSG | DIQMTQS |
| 212 | TQLQLEHLL | GGSGGGG | AGAPSVFLFPPKPK | GGSGGGG | GGLVQPGG | GGGSGG | PSSLSAS |
| | LDLQMILNG | S (SEQ | DTLMISRTPEVTCV | S (SEQ | SLRLSCAA | GGSGGG | VGDRVTI |
| | INNYKNPKL | ID NO: | VVDVSHEDPEVKFN | ID NO: | SGFTFSSY | GS | TCRASQS |
| | TRMLTFKFY | 30) | WYVDGVEVHNAKTK | 30) | WMHWVRQA | (SEQ | ISSSLAW |
| | MPKKATELK | | PREEQYNSTYRVVS | | PGKGLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DMDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1445) | | 1367) |
| MIAB | APTSSSTKK | GGGGSGG | DKTHTCPPCPAPEA | GGGGSGG | EVQLLESG | GGGGSG | DIQMTQS |
| 213 | TQLQLEHLL | GGSGGGG | AGAPSVFLFPPKPK | GGSGGGG | GGLVQPGG | GGGSGG | PSSLSAS |
| | LDLQMILNG | S (SEQ | DTLMISRTPEVTCV | S (SEQ | SLRLSCAA | GGSGGG | VGDRVTI |
| | INNYKNPKL | ID NO: | VVDVSHEDPEVKFN | ID NO: | SGFTFSSY | GS | TCRASQS |
| | TRMLTFKFY | 30) | WYVDGVEVHNAKTK | 30) | WMHWVRQA | (SEQ | ISSSLAW |
| | MPKKATELK | | PREEQYNSTYRVVS | | PGKGLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DIDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1477) | | 1367) |
| MIAB | APTSSSTKK | GGGGSGG | DKTHTCPPCPAPEA | GGGGSGG | EVQLLESG | GGGGSG | DIQMTQS |
| 214 | TQLQLEHLL | GGSGGGG | AGAPSVFLFPPKPK | GGSGGGG | GGLVQPGG | GGGSGG | PSSLSAS |
| | LDLQMILNG | S (SEQ | DTLMISRTPEVTCV | S (SEQ | SLRLSCAA | GGSGGG | VGDRVTI |
| | INNYKNPKL | ID NO: | VVDVSHEDPEVKFN | ID NO: | SGFTFSSY | GS | TCRASQS |
| | TRMLTFKFY | 30) | WYVDGVEVHNAKTK | 30) | WMHWVRQA | (SEQ | ISSSLAW |
| | MPKKATELK | | PREEQYNSTYRVVS | | PGKGLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DLDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1480) | | 1367) |
| MIAB | APTSSSTKK | GGGGSGG | DKTHTCPPCPAPEA | GGGGSGG | EVQLLESG | GGGGSG | DIQMTQS |
| 215 | TQLQLEHLL | GGSGGGG | AGAPSVFLFPPKPK | GGSGGGG | GGLVQPGG | GGGSGG | PSSLSAS |
| | LDLQMILNG | S (SEQ | DTLMISRTPEVTCV | S (SEQ | SLRLSCAA | GGSGGG | VGDRVTI |
| | INNYKNPKL | ID NO: | VVDVSHEDPEVKFN | ID NO: | SGFTFSSY | GS | TCRASQS |
| | TRMLTFKFY | 30) | WYVDGVEVHNAKTK | 30) | WMHWVRQA | (SEQ | ISSSLAW |
| | MPKKATELK | | PREEQYNSTYRVVS | | PGKCLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DMDVWGKG | | KSYPVTF |
| | SIISTLT | | WQQGNVFSCSVMHE | | TTVTVSS | | GCGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1542) | | 1543) |
| MIAB | APTSSSTKK | GGGGSGG | DKTHTCPPCPAPEA | GGGGSGG | EVQLLESG | GGGGSG | DIQMTQS |
| 216 | TQLQLEHLL | GGSGGGG | AGAPSVFLFPPKPK | GGSGGGG | GGLVQPGG | GGGSGG | PSSLSAS |
| | LDLQMILNG | S (SEQ | DTLMISRTPEVTCV | S (SEQ | SLRLSCAA | GGSGGG | VGDRVTI |
| | INNYKNPKL | ID NO: | VVDVSHEDPEVKFN | ID NO: | SGFTFSSY | GS | TCRASQS |
| | TRMLTFKFY | 30) | WYVDGVEVHNAKTK | 30) | WMHWVRQA | (SEQ | ISSSLAW |
| | MPKKATELK | | PREEQYNSTYRVVS | | PGKCLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DIDVWGKG | | KSYPVTF |
| | SIISTLT | | WQQGNVFSCSVMHE | | TTVTVSS | | GCGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1544) | | 1543) |
| MIAB | APTSSSTKK | GGGGSGG | DKTHTCPPCPAPEA | GGGGSGG | EVQLLESG | GGGGSG | DIQMTQS |
| 217 | TQLQLEHLL | GGSGGGG | AGAPSVFLFPPKPK | GGSGGGG | GGLVQPGG | GGGSGG | PSSLSAS |
| | LDLQMILNG | S (SEQ | DTLMISRTPEVTCV | S (SEQ | SLRLSCAA | GGSGGG | VGDRVTI |
| | INNYKNPKL | ID NO: | VVDVSHEDPEVKFN | ID NO: | SGFTFSSY | GS | TCRASQS |
| | TRMLTFKFY | 30) | WYVDGVEVHNAKTK | 30) | WMHWVRQA | (SEQ | ISSSLAW |
| | MPKKATELK | | PREEQYNSTYRVVS | | PGKCLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DLDVWGKG | | KSYPVTF |
| | SIISTLT | | WQQGNVFSCSVMHE | | TTVTVSS | | GCGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1545) | | 1543) |
| MIAB | APTSSSTKK | GGGGSGG | DKTHTCPPCPAPEA | GGGSEGG | EVQLLESG | GGGGSG | DIQMTQS |
| 218 | TQLQLEHLL | GGSGGGG | AGAPSVFLFPPKPK | GSEGGGS | GGLVQPGG | GGGSGG | PSSLSAS |
| | LDLQMILNG | S (SEQ | DTLMISRTPEVTCV | E (SEQ | SLRLSCAA | GGSGGG | VGDRVTI |
| | INNYKNPKL | ID NO: | VVDVSHEDPEVKFN | ID NO: | SGFTFSSY | GS | TCRASQS |
| | TRMLTFKFY | 30) | WYVDGVEVHNAKTK | 1546) | WMHWVRQA | (SEQ | ISSSLAW |
| | MPKKATELK | | PREEQYNSTYRVVS | | PGKGLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DMDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1445) | | 1367) |
| MIAB | APTSSSTKK | GGGGSGG | DKTHTCPPCPAPEA | GGGSEGG | EVQLLESG | GGGGSG | DIQMTQS |
| 219 | TQLQLEHLL | GGSGGGG | AGAPSVFLFPPKPK | GSEGGGS | GGLVQPGG | GGGSGG | PSSLSAS |
| | LDLQMILNG | S (SEQ | DTLMISRTPEVTCV | E (SEQ | SLRLSCAA | GGSGGG | VGDRVTI |
| | INNYKNPKL | ID NO: | VVDVSHEDPEVKFN | ID NO: | SGFTFSSY | GS | TCRASQS |
| | TRMLTFKFY | 30) | WYVDGVEVHNAKTK | 1546) | WMHWVRQA | (SEQ | ISSSLAW |
| | MPKKATELK | | PREEQYNSTYRVVS | | PGKGLEWV | ID NO: | YQQKPGK |
| | HLQCLEEEI | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DIDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1477) | | 1367) |
| MIAB | APTSSSTKK | GGGGSGG | DKTHTCPPCPAPEA | GGGSEGG | EVQLLESG | GGGGSG | DIQMTQS |
| 220 | TQLQLEHLL | GGSGGGG | AGAPSVFLFPPKPK | GSEGGGS | GGLVQPGG | GGGSGG | PSSLSAS |
| | LDLQMILNG | S (SEQ | DTLMISRTPEVTCV | E (SEQ | SLRLSCAA | GGSGGG | VGDRVTI |
| | INNYKNPKL | ID NO: | VVDVSHEDPEVKFN | ID NO: | SGFTFSSY | GS | TCRASQS |
| | TRMLTFKFY | 30) | WYVDGVEVHNAKTK | 1546) | WMHWVRQA | (SEQ | ISSSLAW |
| | MPKKATELK | | PREEQYNSTYRVVS | | PGKGLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DLDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1480) | | 1367) |
| MIAB | APTSSSTKK | GGGGSGG | DKTHTCPPCPAPEA | GGGSEGG | EVQLLESG | GGGGSG | DIQMTQS |
| 221 | TQLQLEHLL | GGSGGGG | AGAPSVFLFPPKPK | GSEGGGS | GGLVQPGG | GGGSGG | PSSLSAS |
| | LDLQMILNG | S (SEQ | DTLMISRTPEVTCV | E (SEQ | SLRLSCAA | GGSGGG | VGDRVTI |
| | INNYKNPKL | ID NO: | VVDVSHEDPEVKFN | ID NO: | SGFTFSSY | GS | TCRASQS |
| | TRMLTFKFY | 30) | WYVDGVEVHNAKTK | 1546) | WMHWVRQA | (SEQ | ISSSLAW |
| | MPKKATELK | | PREEQYNSTYRVVS | | PGKCLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DMDVWGKG | | KSYPVTF |
| | SIISTLT | | WQQGNVFSCSVMHE | | TTVTVSS | | GCGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1542) | | 1543) |
| MIAB | APTSSSTKK | GGGGSGG | DKTHTCPPCPAPEA | GGGSEGG | EVQLLESG | GGGGSG | DIQMTQS |
| 222 | TQLQLEHLL | GGSGGGG | AGAPSVFLFPPKPK | GSEGGGS | GGLVQPGG | GGGSGG | PSSLSAS |
| | LDLQMILNG | S (SEQ | DTLMISRTPEVTCV | E (SEQ | SLRLSCAA | GGSGGG | VGDRVTI |
| | INNYKNPKL | ID NO: | VVDVSHEDPEVKFN | ID NO: | SGFTFSSY | GS | TCRASQS |
| | TRMLTFKFY | 30) | WYVDGVEVHNAKTK | 1546) | WMHWVRQA | (SEQ | ISSSLAW |
| | MPKKATELK | | PREEQYNSTYRVVS | | PGKCLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DIDVWGKG | | KSYPVTF |
| | SIISTLT | | WQQGNVFSCSVMHE | | TTVTVSS | | GCGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1544) | | 1543) |
| MIAB | APTSSSTKK | GGGGSGG | DKTHTCPPCPAPEA | GGGSEGG | EVQLLESG | GGGGSG | DIQMTQS |
| 223 | TQLQLEHLL | GGSGGGG | AGAPSVFLFPPKPK | GSEGGGS | GGLVQPGG | GGGSGG | PSSLSAS |
| | LDLQMILNG | S (SEQ | DTLMISRTPEVTCV | E (SEQ | SLRLSCAA | GGSGGG | VGDRVTI |
| | INNYKNPKL | ID NO: | VVDVSHEDPEVKFN | ID NO: | SGFTFSSY | GS | TCRASQS |
| | TRMLTFKFY | 30) | WYVDGVEVHNAKTK | 1546) | WMHWVRQA | (SEQ | ISSSLAW |
| | MPKKATELK | | PREEQYNSTYRVVS | | PGKCLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DLDVWGKG | | KSYPVTF |
| | SIISTLT | | WQQGNVFSCSVMHE | | TTVTVSS | | GCGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1545) | | 1543) |
| MIAB | APTSSSTKK | GGGGSGG | DKTHTCPPCPAPEA | GGGGSGG | EVQLLESG | GGGGSG | DIQMTQS |
| 224 | TQLQLEHLL | GGSGGGG | AGAPSVFLFPPKPK | GGSGGGG | GGLVQPGG | GGGSGG | PSSLSAS |
| | LDLQMILNG | SGGGGS | DTLMISRTPEVTCV | S (SEQ | SLRLSCAA | GGSGGG | VGDRVTI |
| | INNYKNPKL | (SEQ ID | VVDVSHEDPEVKFN | ID NO: | SGFTFSSY | GS | TCRASQS |
| | TRMLTFKFY | NO: 22) | WYVDGVEVHNAKTK | 30) | WMHWVRQA | (SEQ | ISSSLAW |
| | MPKKATELK | | PREEQYNSTYRVVS | | PGKGLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DMDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1445) | | 1367) |
| MIAB | APTSSSTKK | GGGGSGG | DKTHTCPPCPAPEA | GGGGSGG | EVQLLESG | GGGGSG | DIQMTQS |
| 225 | TQLQLEHLL | GGSGGGG | AGAPSVFLFPPKPK | GGSGGGG | GGLVQPGG | GGGSGG | PSSLSAS |
| | LDLQMILNG | SGGGGS | DTLMISRTPEVTCV | S (SEQ | SLRLSCAA | GGSGGG | VGDRVTI |
| | INNYKNPKL | (SEQ ID | VVDVSHEDPEVKFN | ID NO: | SGFTFSSY | GS | TCRASQS |
| | TRMLTFKFY | NO: 22) | WYVDGVEVHNAKTK | 30) | WMHWVRQA | (SEQ | ISSSLAW |
| | MPKKATELK | | PREEQYNSTYRVVS | | PGKGLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DIDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1477) | | 1367) |
| MIAB | APTSSSTKK | GGGGSGG | DKTHTCPPCPAPEA | GGGGSGG | EVQLLESG | GGGGSG | DIQMTQS |
| 226 | TQLQLEHLL | GGSGGGG | AGAPSVFLFPPKPK | GGSGGGG | GGLVQPGG | GGGSGG | PSSLSAS |
| | LDLQMILNG | SGGGGS | DTLMISRTPEVTCV | S (SEQ | SLRLSCAA | GGSGGG | VGDRVTI |
| | INNYKNPKL | (SEQ ID | VVDVSHEDPEVKFN | ID NO: | SGFTFSSY | GS | TCRASQS |
| | TRMLTFKFY | NO: 22) | WYVDGVEVHNAKTK | 30) | WMHWVRQA | (SEQ | ISSSLAW |
| | MPKKATELK | | PREEQYNSTYRVVS | | PGKGLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DLDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1480) | | 1367) |
| MIAB | APTSSSTKK | GGGGSGG | DKTHTCPPCPAPEA | GGGGSGG | EVQLLESG | GGGGSG | DIQMTQS |
| 227 | TQLQLEHLI | GGSGGGG | AGAPSVFLFPPKPK | GGSGGGG | GGLVQPGG | GGGSGG | PSSLSAS |
| | LDLQMILNG | SGGGGS | DTLMISRTPEVTCV | S (SEQ | SLRLSCAA | GGSGGG | VGDRVTI |
| | INNYKNPKL | (SEQ ID | VVDVSHEDPEVKFN | ID NO: | SGFTFSSY | GS | TCRASQS |
| | TRMLTFKFY | NO: 22) | WYVDGVEVHNAKTK | 30) | WMHWVRQA | (SEQ | ISSSLAW |
| | MPKKATELK | | PREEQYNSTYRVVS | | PGKCLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DMDVWGKG | | KSYPVTF |
| | SIISTLT | | WQQGNVFSCSVMHE | | TTVTVSS | | GCGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1542) | | 1543) |
| MIAB | APTSSSTKK | GGGGSGG | DKTHTCPPCPAPEA | GGGGSGG | EVQLLESG | GGGGSG | DIQMTQS |
| 228 | TQLQLEHLL | GGSGGGG | AGAPSVFLFPPKPK | GGSGGGG | GGLVQPGG | GGGSGG | PSSLSAS |
| | LDLQMILNG | SGGGGS | DTLMISRTPEVTCV | (SEQ | SLRLSCAA | GGSGGG | VGDRVTI |
| | INNYKNPKL | (SEQ ID | VVDVSHEDPEVKFN | ID NO: | SGFTFSSY | GS | TCRASQS |
| | TRMLTFKFY | NO: 22) | WYVDGVEVHNAKTK | 30) | WMHWVRQA | (SEQ | ISSSLAW |
| | MPKKATELK | | PREEQYNSTYRVVS | | PGKCLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DIDVWGKG | | KSYPVTF |
| | SIISTLT | | WQQGNVFSCSVMHE | | TTVTVSS | | GCGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1544) | | 1543) |
| MIAB | APTSSSTKK | GGGGSGG | DKTHTCPPCPAPEA | GGGGSGG | EVQLLESG | GGGGSG | DIQMTQS |
| 229 | TQLQLEHLI | GGSGGGG | AGAPSVFLFPPKPK | GGSGGGG | GGLVQPGG | GGGSGG | PSSLSAS |
| | LDLQMILNG | SGGGGS | DTLMISRTPEVTCV | S (SEQ | SLRLSCAA | GGSGGG | VGDRVTI |
| | INNYKNPKL | (SEQ ID | VVDVSHEDPEVKFN | ID NO: | SGFTFSSY | GS | TCRASQS |
| | TRMLTFKFY | NO: 22) | WYVDGVEVHNAKTK | 30) | WMHWVRQA | (SEQ | ISSSLAW |
| | MPKKATELK | | PREEQYNSTYRVVS | | PGKCLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DLDVWGKG | | KSYPVTF |
| | SIISTLT | | WQQGNVFSCSVMHE | | TTVTVSS | | GCGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1545) | | 1543) |
| MIAB | APTSSSTKK | GGGGSGG | DKTHTCPPCPAPEA | GGGSEGG | EVQLLESG | GGGGSG | DIQMTQS |
| 230 | TQLQLEHLL | GGSGGGG | AGAPSVFLFPPKPK | GSEGGGS | GGLVQPGG | GGGSGG | PSSLSAS |
| | LDLQMILNG | SGGGGS | DTLMISRTPEVTCV | E (SEQ | SLRLSCAA | GGSGGG | VGDRVTI |
| | INNYKNPKL | (SEQ ID | VVDVSHEDPEVKFN | ID NO: | SGFTFSSY | GS | TCRASQS |
| | TRMLTFKFY | NO: 22) | WYVDGVEVHNAKTK | 1546) | WMHWVRQA | (SEQ | ISSSLAW |
| | MPKKATELK | | PREEQYNSTYRVVS | | PGKGLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DMDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1445) | | 1367) |
| MIAB | APTSSSTKK | GGGGSGG | DKTHTCPPCPAPEA | GGGSEGG | EVQLLESG | GGGGSG | DIQMTQS |
| 231 | TQLQLEHLL | GGSGGGG | AGAPSVFLFPPKPK | GSEGGGS | GGLVQPGG | GGGSGG | PSSLSAS |
| | LDLQMILNG | SGGGGS | DTLMISRTPEVTCV | E (SEQ | SLRLSCAA | GGSGGG | VGDRVTI |
| | INNYKNPKL | (SEQ ID | VVDVSHEDPEVKFN | ID NO: | SGFTFSSY | GS | TCRASQS |
| | TRMLTFKFY | NO: 22) | WYVDGVEVHNAKTK | 1546) | WMHWVRQA | (SEQ | ISSSLAW |
| | MPKKATELK | | PREEQYNSTYRVVS | | PGKGLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DIDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1477) | | 1367) |
| MIAB | APTSSSTKK | GGGGSGG | DKTHTCPPCPAPEA | GGGSEGG | EVQLLESG | GGGGSG | DIQMTQS |
| 232 | TQLQLEHLL | GGSGGGG | AGAPSVFLFPPKPK | GSEGGGS | GGLVQPGG | GGGSGG | PSSLSAS |
| | LDLQMILNG | SGGGGS | DTLMISRTPEVTCV | E (SEQ | SLRLSCAA | GGSGGG | VGDRVTI |
| | INNYKNPKL | (SEQ ID | VVDVSHEDPEVKFN | ID NO: | SGFTFSSY | GS | TCRASQS |
| | TRMLTFKFY | NO: 22) | WYVDGVEVHNAKTK | 1546) | WMHWVRQA | (SEQ | ISSSLAW |
| | MPKKATELK | | PREEQYNSTYRVVS | | PGKGLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DLDVWGKG | | KSYPVTF |
| | SIISTLT | | WQQGNVFSCSVMHE | | TTVTVSS | | GQGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1480) | | 1367) |
| MIAB | APTSSSTKK | GGGGSGG | DKTHTCPPCPAPEA | GGGSEGG | EVQLLESG | GGGGSG | DIQMTQS |
| 233 | TQLQLEHLL | GGSGGGG | AGAPSVFLFPPKPK | GSEGGGS | GGLVQPGG | GGGSGG | PSSLSAS |
| | LDLQMILNG | SGGGGS | DTLMISRTPEVTCV | E (SEQ | SLRLSCAA | GGSGGG | VGDRVTI |
| | INNYKNPKL | (SEQ ID | VVDVSHEDPEVKFN | ID NO: | SGFTFSSY | GS | TCRASQS |
| | TRMLTFKFY | NO: 22) | WYVDGVEVHNAKTK | 1546) | WMHWVRQA | (SEQ | ISSSLAW |
| | MPKKATELK | | PREEQYNSTYRVVS | | PGKCLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DMDVWGKG | | KSYPVTF |
| | SIISTLT | | WQQGNVFSCSVMHE | | TTVTVSS | | GCGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1542) | | 1543) |
| MIAB | APTSSSTKK | GGGGSGG | DKTHTCPPCPAPEA | GGGSEGG | EVQLLESG | GGGGSG | DIQMTQS |
| 234 | TQLQLEHLL | GGSGGGG | AGAPSVFLFPPKPK | GSEGGGS | GGLVQPGG | GGGSGG | PSSLSAS |
| | LDLQMILNG | SGGGGS | DTLMISRTPEVTCV | E (SEQ | SLRLSCAA | GGSGGG | VGDRVTI |
| | INNYKNPKL | (SEQ ID | VVDVSHEDPEVKFN | ID NO: | SGFTFSSY | GS | TCRASQS |
| | TRMLTFKFY | NO: 22) | WYVDGVEVHNAKTK | 1546) | WMHWVRQA | (SEQ | ISSSLAW |
| | MPKKATELK | | PREEQYNSTYRVVS | | PGKCLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DIDVWGKG | | KSYPVTF |
| | SIISTLT | | WQQGNVFSCSVMHE | | TTVTVSS | | GCGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1544) | | 1543) |
| MIAB | APTSSSTKK | GGGGSGG | DKTHTCPPCPAPEA | GGGSEGG | EVQLLESG | GGGGSG | DIQMTQS |
| 235 | TQLQLEHLL | GGSGGGG | AGAPSVFLFPPKPK | GSEGGGS | GGLVQPGG | GGGSGG | PSSLSAS |
| | LDLQMILNG | SGGGGS | DTLMISRTPEVTCV | E (SEQ | SLRLSCAA | GGSGGG | VGDRVTI |
| | INNYKNPKL | (SEQ ID | VVDVSHEDPEVKFN | ID NO: | SGFTFSSY | GS | TCRASQS |
| | TRMLTFKFY | NO: 22) | WYVDGVEVHNAKTK | 1546) | WMHWVRQA | (SEQ | ISSSLAW |
| | MPKKATELK | | PREEQYNSTYRVVS | | PGKCLEWV | ID NO: | YQQKPGK |
| | HLQCLEEEL | | VLTVLHQDWLNGKE | | SYISGSGG | 22) | APKLLIY |
| | KPLEEALNL | | YKCKVSNKALPAPI | | YTNYADSV | | AASSLQS |
| | APSKNFHLR | | EKTISKAKGQPREP | | KGRFTISR | | GVPSRFS |
| | PRDLISDIN | | QVYTLPPSREEMTK | | DNSKNTLY | | GSGSGTD |
| | VIVLELKGS | | NQVSLTCLVKGFYP | | LQMNSLRA | | FTLTISS |
| | ETTFMCEYA | | SDIAVEWESNGQPE | | EDTAVYYC | | LQPEDFA |
| | DETATIVEF | | NNYKTTPPVLDSDG | | ARDRPYYY | | TYYCQQY |
| | INRWITFSQ | | SFFLYSKLTVDKSR | | DLDVWGKG | | KSYPVTF |
| | SIISTLT | | WQQGNVFSCSVMHE | | TTVTVSS | | GCGTKVE |
| | (SEQ ID | | ALHNHYTQKSLSLS | | (SEQ ID | | IK (SEQ |
| | NO: 41) | | PG (SEQ ID NO: | | NO: | | ID NO: |
| | | | 21) | | 1545) | | 1543) |

In some embodiments, the MAdCAM antibody comprises one or more sequences, or a combination thereof, of the sequences presented in Table 11.

In some embodiments, the polypeptide is referred to as an antibody or antigen binding protein.

In some embodiments, as provided for herein, the MAdCAM antibody, or binding fragment thereof, is linked directly or indirectly to a PD-1 antibody or binding fragment thereof.

In some embodiments, as provided for herein, the MAdCAM antibody, or binding fragment thereof, is linked directly or indirectly to an IL-2 mutein or binding fragment thereof. The IL-2 mutein can be any mutein as provided for herein or other IL-2 muteins known to one of skill in the art.

In some embodiments, if the therapeutic compound comprises a Fc portion, the Fc domain, (portion) bears mutations to render the Fc region “effectorless,” that is unable to bind FcRs. The mutations that render Fc regions effectorless are known. In some embodiments, the mutations in the Fc region, which is according to the known numbering system, are selected from the group consisting of: K322A, L234A, L235A, G237A, L234F, L235E, N297, P331S, or any combination thereof. In some embodiments, the Fc mutations comprises a mutation at L234 and/or L235 and/or G237. In some embodiments, the Fc mutations comprise L234A and/or L235A mutations, which can be referred to as LALA mutations. In some embodiments, the Fc mutations comprise L234A, L235A, and G237A mutations.

Disclosed herein are Linker Region polypeptides, therapeutic peptides, and nucleic acids encoding the polypeptides (e.g. therapeutic compounds), vectors comprising the nucleic acid sequences, and cells comprising the nucleic acids or vectors

Therapeutic compounds can comprise a plurality of specific targeting moieties. In some embodiments, the therapeutic compound comprises a plurality one specific targeting moiety, a plurality of copies of a donor specific targeting moiety or a plurality of tissue specific targeting moieties. In some embodiments, a therapeutic compound comprises a first and a second donor specific targeting moiety, e.g., a first donor specific targeting moiety specific for a first donor target and a second donor specific targeting moiety specific for a second donor target, e.g., wherein the first and second target are found on the same donor tissue. In some embodiments, the therapeutic compound comprises e.g., a first specific targeting moiety for a tissue specific target and a second specific targeting moiety for a second target, e.g., wherein the first and second target are found on the same or different target tissue.

In some embodiments, a therapeutic compound comprises a plurality of effector binding/modulating moieties each comprising an ICIM binding/modulating moiety, the number of ICIM binding/modulating moieties is sufficiently low that clustering of the ICIM binding/modulating moiety's ligand on immune cells (in the absence of target binding) is minimized, e.g., to avoid systemic agonizing of immune cells in the absence of binding of the therapeutic compound to target.

In some embodiments, the therapeutic compound has the formula from N-terminus to C-terminus:
A1-Linker A-A2-Linker B-A3
A3-Linker A-A2-Linker B-A1,
   wherein,
A1 and A3, each independently comprises an effector binding/modulating moiety, e.g., an ICIM binding/modulating moiety, an IIC binding/modulating moiety, ICSM binding/modulating moiety, or an SM binding/modulating moiety; or a specific targeting moiety,
A2 comprises an Fc region or is absent; and
Linker A and Linker B, each are independent linkers.

In some embodiments,
A1 comprises an IL-2 mutein molecule,
A3 comprises a specific targeting moiety, e.g. anti-human MAdCAM Ab, such as a scFv,
A2 comprises an Fc region, and
Linker A and Linker B, each are independent linkers further comprising glycine/serine linkers.

In some embodiments, a polypeptide is provided, wherein the polypeptide comprises a peptide of the formula

Ab-ConstantDomain-LinkerA-IL2Mutein-LinkerB-FcRegion, wherein the Ab is a variable heavy chain domain that binds to MAdCAM, the Constant domain is an Ig constant domain such as IgG1, IgG2, IgG3, or IgG4, Linker A is a linker, such as those provided herein, and the IL2Mutein is an IL-2 mutein, such as those provided for herein. In some embodiments, the variable heavy domain is a variable heavy chain domain as illustrated in Table 7. In some embodiments, the variable heavy chain domain comprises the variable heavy chain domain of Clone ID: 6, 75, or 79 of Table 7; MIAB197 of Table 9, or MIAB204 of Table 11. In some embodiments, the variable heavy chain domain comprises the CDRs of the heavy domain of 6, 75, or 79 of Table 7; or MIAB197 of Table 9. In some embodiments, the VH comprises a sequence of SEQ ID NO: 414, SEQ ID NO: 591, SEQ ID NO: 599, and SEQ ID NO: 1387.

In some embodiments, the ConstantDomain comprises a IgG1 constant domain, such as those provided for herein. In some embodiments, the constant domain comprises mutations to render the constant region “effectorless,” that is unable to bind FcRs. The mutations that render constant regions effectorless are known. In some embodiments, the mutations in the constant region, which is according to the known numbering system, are selected from the group consisting of: K322A, L234A, L235A, G237A, L234F, L235E, N297, P331S, or any combination thereof. In some embodiments, the constant region mutations comprises a mutation at L234 and/or L235 and/or G237. In some embodiments, the constant region mutations comprise L234A and/or L235A mutations, which can be referred to as LALA mutations. In some embodiments, the constant region mutations comprise L234A, L235A, and G237A mutations. In some embodiments, the ConstantDomain comprises SEQ ID NO: 44.

In some embodiments, the MAdCAM antibody is selected from the following table:

TABLE 15

| | ScFv | ScFv | | | | | | |
|---|---|---|---|---|---|---|---|---|
| Clone | VH | VK | | | | | | |
| (scFv) | Seq | Seq | HCDR1 | HCDR2 | HCDR3 | LCDR1 | LCDR2 | LCDR3 |
| MIAB212 | EVQLL | DIQMT | FTFSS | SYISG | CARDR | RASQS | SSLQS | QQYKS |
| | ESGGG | QSPS | YWMH | SGGYT | PYYYD | ISSSL | (SEQ | YPVT |
| | LVQPG | SLSAS | (SEQ | NYA | MDVW | A | ID | (SEQ |
| | GSLRL | VGDR | ID | (SEQ | (SEQ | (SEQ | NO: | ID |
| | SCAAS | VTITC | NO: | ID | ID | ID | 1497) | NO: |
| | GFTFS | RASQ | 1499) | NO: | NO: | NO: | | 1498) |
| | SYWMH | SISSS | | 1506) | 1507) | 1502) | | |
| | WVRQA | LAWY | | | | | | |
| | PGKGL | QQKPG | | | | | | |
| | EWVSY | KAPK | | | | | | |
| | ISGSG | LLIYA | | | | | | |
| | GYTNY | ASSL | | | | | | |
| | ADSVK | QSGVP | | | | | | |
| | GRFTI | SRFS | | | | | | |
| | SRDNS | GSGSG | | | | | | |
| | KNTLY | TDFT | | | | | | |
| | LQMNS | LTISS | | | | | | |
| | LRAED | LQPE | | | | | | |
| | TAVYY | DFATY | | | | | | |
| | CARDR | YCQQ | | | | | | |
| | PYYYD | YKSYP | | | | | | |
| | MDVWG | VTFG | | | | | | |
| | KGTTV | QGTKV | | | | | | |
| | TVSS | EIK | | | | | | |
| | (SEQ | (SEQ | | | | | | |
| | ID | ID | | | | | | |
| | NO: | NO: | | | | | | |
| | 1445) | 1367) | | | | | | |
| MIAB213 | EVQLL | DIQMT | FTFSS | SYISG | CARDR | RASQS | SSLQS | QQYKS |
| | ESGGG | QSPS | YWMH | SGGYT | PYYYD | ISSSL | (SEQ | YPVT |
| | LVQPG | SLSAS | (SEQ | NYA | IDVW | A | ID | (SEQ |
| | GSLRL | VGDR | ID | (SEQ | (SEQ | (SEQ | NO: | ID |
| | SCAAS | VTITC | NO: | ID | ID | ID | 1497) | NO: |
| | GFTFS | RASQ | 1499) | NO: | NO: | NO: | | 1498) |
| | SYWMH | SISSS | | 1506) | 1531) | 1502) | | |
| | WVRQA | LAWY | | | | | | |
| | PGKGL | QQKPG | | | | | | |
| | EWVSY | KAPK | | | | | | |
| | ISGSG | LLIYA | | | | | | |
| | GYTNY | ASSL | | | | | | |
| | ADSVK | QSGVP | | | | | | |
| | GRFTI | SRFS | | | | | | |
| | SRDNS | GSGSG | | | | | | |
| | KNTLY | TDFT | | | | | | |
| | LQMNS | LTISS | | | | | | |
| | LRAED | LQPE | | | | | | |
| | TAVYYC | DFATY | | | | | | |
| | ARDR | YCQQ | | | | | | |
| | PYYYDI | YKSYP | | | | | | |
| | DVWG | VTFG | | | | | | |
| | KGTTVT | QGTKV | | | | | | |
| | VSS | EIK | | | | | | |
| | (SEQ | (SEQ | | | | | | |
| | ID | ID | | | | | | |
| | NO: | NO: | | | | | | |
| | 1477) | 1367) | | | | | | |
| MIAB214 | EVQLL | DIQMT | FTFSS | SYISG | CARDR | RASQS | SSLQS | QQYKS |
| | ESGGG | QSPS | YWMH | SGGYT | PYYYD | ISSSL | (SEQ | YPVT |
| | LVQPG | SLSAS | (SEQ | NYA | LDVW | A | ID | (SEQ |
| | GSLRL | VGDR | ID | (SEQ | (SEQ | (SEQ | NO: | ID |
| | SCAAS | VTITC | NO: | ID | ID | ID | 1497) | NO: |
| | GFTFS | RASQ | 1499) | NO: | NO: | NO: | | 1498) |
| | SYWMH | SISSS | | 1506) | 1532) | 1502) | | |
| | WVRQA | LAWY | | | | | | |
| | PGKGL | QQKPG | | | | | | |
| | EWVSY | KAPK | | | | | | |
| | ISGSG | LLIYA | | | | | | |
| | GYTNY | ASSL | | | | | | |
| | ADSVK | QSGVP | | | | | | |
| | GRFTI | SRFS | | | | | | |
| | SRDNS | GSGSG | | | | | | |
| | KNTLY | TDFT | | | | | | |
| | LQMNS | LTISS | | | | | | |
| | LRAED | LQPE | | | | | | |
| | TAVYY | DFATY | | | | | | |
| | CARDR | YCQQ | | | | | | |
| | PYYYD | YKSYP | | | | | | |
| | LDVWG | VTFG | | | | | | |
| | KGTTV | QGTKV | | | | | | |
| | TVSS | EIK | | | | | | |
| | (SEQ | (SEQ | | | | | | |
| | ID | ID | | | | | | |
| | NO: | NO: | | | | | | |
| | 1480) | 1367) | | | | | | |

In some embodiments, the variable heavy chain domain comprises a first CDR of SEQ ID NO: 90, a second CDR of SEQ ID NO: 91, and a third CDR of SEQ ID NO: 92. In some embodiments, the variable heavy chain domain comprises a first CDR of SEQ ID NO: 359, a second CDR of SEQ ID NO: 170, and a third CDR of SEQ ID NO: 360. In some embodiments, the variable heavy chain domain comprises a first CDR of SEQ ID NO: 135, a second CDR of SEQ ID NO: 381, and a third CDR of SEQ ID NO: 382. In some embodiments, the variable heavy chain domain comprises a first CDR of SEQ ID NO: 359, a second CDR of SEQ ID NO: 170, and a third CDR of SEQ ID NO: 1431. These are illustrative only and the CDR sets as set forth herein and in the tables are also provided.

In some embodiments, the LinkerA is a glycine/serine linker, which can be any glycine/serine linker provided for herein. In some embodiments, the linker is a sequence of GGGGSGGGGSGGGGSGGGGS (SEQ ID NO: 22) or GGGGSGGGGSGGGGS (SEQ ID NO: 30). These are non-limiting examples and the linker can have varying number of GGGGS (SEQ ID NO: 23) or GGGGA repeats (SEQ ID NO: 29), or a mixture of the two. In some embodiments, the linker comprises 1, 2, 3, 4, 5, 6, 7, 8, 9, or 10 of the GGGGS (SEQ ID NO: 23) and/or GGGGA repeats (SEQ ID NO: 29) repeats (repeats disclosed as SEQ ID NOS 1550-1551, respectively). In some embodiments, the linker is 10 amino acids in length. In some embodiments, the linker is 5 amino acids in length. In some embodiments, the linker is 15 amino acids in length. In some embodiments, the linker is 20 amino acids in length. In some embodiments, the linker is 25 amino acids in length. In some embodiments, the linker is 30 amino acids in length. In some embodiments, the linker is 35 amino acids in length. In some embodiments, the linker is from 5-50 amino acids in length.

In some embodiments, the IL-2 mutein comprises a sequence of SEQ ID NO: 31. In some embodiments, the IL-2 mutein comprises a sequence of SEQ ID NO: 32. In some embodiments, the IL-2 mutein comprises a sequence of SEQ ID NO: 33. In some embodiments, the IL-2 mutein comprises a sequence of SEQ ID NO: 34. In some embodiments, the IL-2 mutein comprises a sequence of SEQ ID NO: 35. In some embodiments, the IL-2 mutein comprises a sequence of SEQ ID NO: 36. In some embodiments, the IL-2 mutein comprises a sequence of SEQ ID NO: 37. In some embodiments, the IL-2 mutein comprises a sequence of SEQ ID NO: 38. In some embodiments, the IL-2 mutein comprises a sequence of SEQ ID NO: 39. In some embodiments, the IL-2 mutein comprises a sequence of SEQ ID NO: 40. In some embodiments, the IL-2 mutein comprises a sequence of SEQ ID NO: 41. In some embodiments, the IL-2 mutein further comprises a T3A substitution (mutation). In some embodiments, the Fc Region comprises a peptide having a sequence of SEQ ID NO: 21. In some embodiments, the Fc Region comprises a peptide having a sequence of SEQ ID NO: 28. In some embodiments, the C-terminus of the Fc Region is linked to the N-terminus or the C-terminus of the variable heavy chain or IL-2 mutein. In some embodiments, the linker linking the Fc Region to the variable heavy chain or the IL-2 mutein is a glycine/serine or a glycine/alanine linker. In some embodiments, the linker linking the Fc region to the C- or N-terminus of the variable heavy chain or TL-2 mutein is a glycine/serine linker, which can be a sequence of GGGGSGGGGSGGGGSGGGGS (SEQ ID NO: 22) or GGGGSGGGGSGGGGS (SEQ ID NO: 30). These are non-limiting examples and the linker can have varying number of GGGGS (SEQ ID NO: 23) or GGGGA (SEQ ID NO: 29) repeats, or a mixture of the two. In some embodiments, the linker comprises 1, 2, 3, 4, 5, 6, 7, 8, 9, or 10 of the GGGGS (SEQ ID NO: 23) and/or GGGGA (SEQ ID NO: 29) repeats (repeats disclosed as SEQ ID NOS 1550-1551, respectively). In some embodiments, the linker is 10 amino acids in length. In some embodiments, the linker is 5 amino acids in length. In some embodiments, the linker is 15 amino acids in length. In some embodiments, the linker is 20 amino acids in length. In some embodiments, the linker is 25 amino acids in length. In some embodiments, the linker is 30 amino acids in length. In some embodiments, the linker is 35 amino acids in length. In some embodiments, the linker is from 5-50 amino acids in length.

In some embodiments, the polypeptide further comprises a polypeptide of formula VL-ConstantDomainLight, wherein VL is a variable light chain and ConstantDomainLight is an IgG light chain constant domain, wherein the polypeptide can be or is associated with the polypeptide having the formula of Ab-ConstantDomain-LinkerA-IL2Mutein-LinkerB-FcRegion. In some embodiments, the VL comprises a sequence of SEQ ID NO: 415, SEQ ID NO: 592, SEQ ID NO: 600 or SEQ ID NO: 1363. These are illustrative only and the VL domain can be VL/VK sequence provided for herein, such as in Table 7 or Table 9. In some embodiments, the variable light chain domain comprises a first CDR of SEQ ID NO: 93, a second CDR of SEQ ID NO: 87, and a third CDR of SEQ ID NO: 94. In some embodiments, the variable light chain domain comprises a first CDR of SEQ ID NO: 361, a second CDR of SEQ ID NO: 362, and a third CDR of SEQ ID NO: 363. In some embodiments, the variable heavy chain domain comprises a first CDR of SEQ ID NO: 383, a second CDR of SEQ ID NO: 384, and a third CDR of SEQ ID NO: 385. In some embodiments, the variable heavy chain domain comprises a first CDR of SEQ ID NO: 1408, a second CDR of SEQ ID NO: 362, and a third CDR of SEQ ID NO: 363. These are illustrative only and the CDR sets as set forth herein and in the tables are also provided.

In some embodiments, the constant domain also comprises mutations to negate the effector function, such as those provided for herein. In some embodiments, the ConstantDomainLight comprises a sequence of:

(SEQ ID NO: 45)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

The different polypeptides of formula IL2Mutein-LinkerA-FcRegion-LinkerB-Ab and VL-ConstantDomainLight can be interchanged with one another. In some embodiments, the polypeptide comprises a variable heavy chain comprising a first CDR of SEQ ID NO: 90, a second CDR of SEQ ID NO: 91, and a third CDR of SEQ ID NO: 92 and a variable light chain comprising a first CDR of SEQ ID NO: 93, a second CDR of SEQ ID NO: 87, and a third CDR of SEQ ID NO: 94. In some embodiments, the polypeptide comprises a variable heavy chain comprising a first CDR of SEQ ID NO: 359, a second CDR of SEQ ID NO: 170, and a third CDR of SEQ ID NO: 360 and a variable light chain comprising a first CDR of SEQ ID NO: 361, a second CDR of SEQ ID NO: 362, and a third CDR of SEQ ID NO: 363. In some embodiments, the polypeptide comprises a variable heavy chain comprising a first CDR of SEQ ID NO: 135, a second CDR of SEQ ID NO: 381, and a third CDR of SEQ ID NO: 382 and a variable light chain comprising a first CDR of SEQ ID NO: 383, a second CDR of SEQ ID NO: 384, and a third CDR of SEQ ID NO: 385. In some embodiments, the polypeptide comprises a variable heavy chain comprising a first CDR of SEQ ID NO: 359, a second CDR of SEQ ID NO: 170, and a third CDR of SEQ ID NO: 1431; and a variable light chain comprising a first CDR of SEQ ID NO: 1408, a second CDR of SEQ ID NO: 362, and a third CDR of SEQ ID NO: 363. These are non-limiting examples and the CDR combinations as illustrated in the Table 9 and Table 14 can be also be used and are provided for herein.

In some embodiments, compounds are provided comprising the following formula, from N-terminus to C-terminus:

IL2Mutein-LinkerA-FcRegion-LinkerB-Ab, wherein the IL2Mutein is any IL-2 mutein that can, for example, preferentially activate Tregs; the Linker A and Linker B are, each, independently, a linker as provided herein, the Fc Region can any one of such as provided herein, and the Ab is a tissue targeting moiety, such as those provided herein. In some embodiments, the Ab is an antibody that binds to MAdCAM or another cell surface target as provided herein. In some embodiments, the antibody is in a scFV format. In some embodiments, the antibody in scFV format is an antibody as provided in the Table 6 or Table 14. In some embodiments, the antibody in scFV format is an antibody that comprises the CDRs as set forth in Table 6, Table 7, Table 11, or Table 14.

In some embodiments, the C-terminus of the IL-2 mutein is linked to the N-terminus of the Fc region. In some embodiments, the linkage is direct or through a linker, such as those described herein. In some embodiments, the linker is a glycine/serine linker. In some embodiments, the linker linking the IL-2 mutein to the Fc region is a glycine/serine linker. In some embodiments, the glycine/serine linker comprises or consists of a sequence of GGGGSGGGGSGGGGSGGGGS (SEQ ID NO: 22) or GGGGSGGGGSGGGGS (SEQ ID NO: 30). These are non-limiting examples and the linker can have varying number of GGGGS (SEQ ID NO: 23) or GGGGA (SEQ ID NO: 29) repeats, or a mixture of the two. In some embodiments, the linker comprises 1, 2, 3, 4, 5, 6, 7, 8, 9, or 10 of the GGGGS (SEQ ID NO: 23) and/or GGGGA (SEQ ID NO: 29) repeats (repeats disclosed as SEQ ID NOS 1550-1551, respectively). In some embodiments, the linker is 10 amino acids in length. In some embodiments, the linker is 5 amino acids in length. In some embodiments, the linker is 15 amino acids in length. In some embodiments, the linker is 20 amino acids in length. In some embodiments, the linker is 25 amino acids in length. In some embodiments, the linker is 30 amino acids in length. In some embodiments, the linker is 35 amino acids in length. In some embodiments, the linker is from 5-50 amino acids in length.

In some embodiments, the IL-2 mutein comprises a sequence of SEQ ID NO: 31. In some embodiments, the IL-2 mutein comprises a sequence of SEQ ID NO: 32. In some embodiments, the IL-2 mutein comprises a sequence of SEQ ID NO: 33. In some embodiments, the IL-2 mutein comprises a sequence of SEQ ID NO: 34. In some embodiments, the IL-2 mutein comprises a sequence of SEQ ID NO: 35. In some embodiments, the IL-2 mutein comprises a sequence of SEQ ID NO: 36. In some embodiments, the IL-2 mutein comprises a sequence of SEQ ID NO: 37. In some embodiments, the IL-2 mutein comprises a sequence of SEQ ID NO: 38. In some embodiments, the IL-2 mutein comprises a sequence of SEQ ID NO: 39. In some embodiments, the IL-2 mutein comprises a sequence of SEQ ID NO: 40. In some embodiments, the IL-2 mutein comprises a sequence of SEQ ID NO: 41. In some embodiments, the IL-2 mutein further comprises a T3A substitution (mutation). In some embodiments, the Fc Region comprises a peptide having a sequence of SEQ ID NO: 21. In some embodiments, the Fc Region comprises a peptide having a sequence of SEQ ID NO: 28. In some embodiments, the C-terminus of the Fc Region is linked to the N-terminus of the variable heavy chain. In some embodiments, the linker linking the Fc Region to the variable heavy chain is a glycine/serine or a glycine/alanine linker. In some embodiments, the linker linking the Fc region to the N-terminus of the variable heavy chain is a glycine/serine linker, which can be a sequence of GGGGSGGGGSGGGGSGGGGS (SEQ ID NO: 22) or GGGGSGGGGSGGGGS (SEQ ID NO: 30). These are non-limiting examples and the linker can have varying number of GGGGS (SEQ ID NO: 23) or GGGGA (SEQ ID NO: 29) repeats, or a mixture of the two. In some embodiments, the linker comprises 1, 2, 3, 4, 5, 6, 7, 8, 9, or 10 of the GGGGS (SEQ ID NO: 23) and/or GGGGA (SEQ ID NO: 29) repeats (repeats disclosed as SEQ ID NOS 1550-1551, respectively). In some embodiments, the linker is 10 amino acids in length. In some embodiments, the linker is 5 amino acids in length. In some embodiments, the linker is 15 amino acids in length. In some embodiments, the linker is 20 amino acids in length. In some embodiments, the linker is 25 amino acids in length. In some embodiments, the linker is 30 amino acids in length. In some embodiments, the linker is 35 amino acids in length. In some embodiments, the linker is from 5-50 amino acids in length.

In some embodiments, the variable heavy chain comprises the CDRs as set forth in Table 6, Table 7, Table 9, or Table 14. In some embodiments, the variable heavy chain comprises a HCDR1, HCDR2, and a HCDR3, wherein the HCDR1, HCDR2, and a HCDR3 are as set forth in Table 6, Table 7, Table 9, or Table 14. In some embodiments, the variable heavy chain has a HCDR1, HCDR2, and a HCDR3 as set forth for Clone 1 in Table 6. In some embodiments, the variable heavy chain has a HCDR1, HCDR2, and a HCDR3 as set forth for Clone 2 in Table 6. In some embodiments, the variable heavy chain has a HCDR1, HCDR2, and a HCDR3 as set forth for Clone 3 in Table 6. In some embodiments, the variable heavy chain has a HCDR1, HCDR2, and a HCDR3 as set forth for Clone 4 in Table 6. In some embodiments, the variable heavy chain has a HCDR1, HCDR2, and a HCDR3 as set forth for Clone 5 in Table 6. In some embodiments, the variable heavy chain has a HCDR1, HCDR2, and a HCDR3 as set forth for Clone 6 in Table 6. In some embodiments, the variable heavy chain has a HCDR1, HCDR2, and a HCDR3 as set forth for Clone 7 in Table 6. In some embodiments, the variable heavy chain has a HCDR1, HCDR2, and a HCDR3 as set forth for Clone 8 in Table 6. In some embodiments, the variable heavy chain has a HCDR1, HCDR2, and a HCDR3 as set forth for Clone 9 in Table 6. In some embodiments, the variable heavy chain has a HCDR1, HCDR2, and a HCDR3 as set forth for Clone 10 in Table 6. In some embodiments, the variable heavy chain has a HCDR1, HCDR2, and a HCDR3 as set forth for Clone 11 in Table 6. In some embodiments, the variable heavy chain has a HCDR1, HCDR2, and a HCDR3 as set forth for Clone 12 in Table 6. In some embodiments, the variable heavy chain has a HCDR1, HCDR2, and a HCDR3 as set forth for Clone 13 in Table 6. In some embodiments, the variable heavy chain has a HCDR1, HCDR2, and a HCDR3 as set forth for Clone 14 in Table 6. In some embodiments, the variable heavy chain has a HCDR1, HCDR2, and a HCDR3 as set forth for Clone 15 in Table 6. In some embodiments, the variable heavy chain has a HCDR1, HCDR2, and a HCDR3 as set forth for Clone 16 in Table 6. In some embodiments, the variable heavy chain has a HCDR1, HCDR2, and a HCDR3 as set forth for Clone 17 in Table 6. In some embodiments, the variable heavy chain has a HCDR1, HCDR2, and a HCDR3 as set forth for Clone 1 in Table 6. In some embodiments, the variable heavy chain has a HCDR1, HCDR2, and a HCDR3 as set forth for Clone 18 in Table 6. In some embodiments, the variable heavy chain has a HCDR1, HCDR2, and a HCDR3 as set forth for Clone 19 in Table 6. In some embodiments, the variable heavy chain has a HCDR1, HCDR2, and a HCDR3 as set forth for Clone 20 in Table 6. In some embodiments, the variable heavy chain has a HCDR1, HCDR2, and a HCDR3 as set forth for Clone 21 in Table 6. In some embodiments, the variable heavy chain has a HCDR1, HCDR2, and a HCDR3 as set forth for Clone 22 in Table 6. In some embodiments, the variable heavy chain has a HCDR1, HCDR2, and a HCDR3 as set forth for Clone 23 in Table 6. In some embodiments, the variable heavy chain has a HCDR1, HCDR2, and a HCDR3 as set forth for Clone 24 in Table 6. In some embodiments, the variable heavy chain has a HCDR1, HCDR2, and a HCDR3 as set forth for Clone 25 in Table 6. In some embodiments, the variable heavy chain has a HCDR1, HCDR2, and a HCDR3 as set forth for Clone 26 in Table 6. In some embodiments, the variable heavy chain has a HCDR1, HCDR2, and a HCDR3 as set forth for Clone 27 in Table 6. In some embodiments, the variable heavy chain has a HCDR1, HCDR2, and a HCDR3 as set forth for Clone 28 in Table 6. In some embodiments, the variable heavy chain has a HCDR1, HCDR2, and a HCDR3 as set forth for Clone 29 in Table 6. In some embodiments, the variable heavy chain has a HCDR1, HCDR2, and a HCDR3 as set forth for Clone 30 in Table 6. In some embodiments, the variable heavy chain has a HCDR1, HCDR2, and a HCDR3 as set forth for Clone 31 in Table 6. In some embodiments, the variable heavy chain has a HCDR1, HCDR2, and a HCDR3 as set forth for Clone 32 in Table 6. In some embodiments, the variable heavy chain has a HCDR1, HCDR2, and a HCDR3 as set forth for Clone 33 in Table 6. In some embodiments, the variable heavy chain has a HCDR1, HCDR2, and a HCDR3 as set forth for Clone 34 in Table 6. In some embodiments, the variable heavy chain has a HCDR1, HCDR2, and a HCDR3 as set forth for Clone 35 in Table 6. In some embodiments, the variable heavy chain has a HCDR1, HCDR2, and a HCDR3 as set forth for Clone 36 in Table 6. In some embodiments, the variable heavy chain has a HCDR1, HCDR2, and a HCDR3 as set forth for Clone 37 in Table 6. In some embodiments, the variable heavy chain has a HCDR1, HCDR2, and a HCDR3 as set forth for Clone 38 in Table 6. In some embodiments, the variable heavy chain has a HCDR1, HCDR2, and a HCDR3 as set forth for Clone 39 in Table 6. In some embodiments, the variable heavy chain has a HCDR1, HCDR2, and a HCDR3 as set forth for Clone 40 in Table 6. In some embodiments, the variable heavy chain has a HCDR1, HCDR2, and a HCDR3 as set forth for Clone 41 in Table 6. In some embodiments, the variable heavy chain has a HCDR1, HCDR2, and a HCDR3 as set forth for Clone 42 in Table 6. In some embodiments, the variable heavy chain has a HCDR1, HCDR2, and a HCDR3 as set forth for Clone 43 in Table 6. In some embodiments, the variable heavy chain has a HCDR1, HCDR2, and a HCDR3 as set forth for Clone 44 in Table 6. In some embodiments, the variable heavy chain has a HCDR1, HCDR2, and a HCDR3 as set forth for Clone 45 in Table 6. In some embodiments, the variable heavy chain has a HCDR1, HCDR2, and a HCDR3 as set forth for Clone 46 in Table 6. In some embodiments, the variable heavy chain has a HCDR1, HCDR2, and a HCDR3 as set forth for Clone 47 in Table 6. In some embodiments, the variable heavy chain has a HCDR1, HCDR2, and a HCDR3 as set forth for Clone 48 in Table 6. In some embodiments, the variable heavy chain has a HCDR1, HCDR2, and a HCDR3 as set forth for Clone 49 in Table 6. In some embodiments, the variable heavy chain has a HCDR1, HCDR2, and a HCDR3 as set forth for Clone 50 in Table 6. In some embodiments, the variable heavy chain has a HCDR1, HCDR2, and a HCDR3 as set forth for Clone 51 in Table 6. In some embodiments, the variable heavy chain has a HCDR1, HCDR2, and a HCDR3 as set forth for Clone 52 in Table 6. In some embodiments, the variable heavy chain has a HCDR1, HCDR2, and a HCDR3 as set forth for Clone 53 in Table 6. In some embodiments, the variable heavy chain has a HCDR1, HCDR2, and a HCDR3 as set forth for Clone 54 in Table 6. In some embodiments, the variable heavy chain has a HCDR1, HCDR2, and a HCDR3 as set forth for Clone 55 in Table 6. In some embodiments, the variable heavy chain has a HCDR1, HCDR2, and a HCDR3 as set forth for Clone 56 in Table 6. In some embodiments, the variable heavy chain has a HCDR1, HCDR2, and a HCDR3 as set forth for Clone 57 in Table 6. In some embodiments, the variable heavy chain has a HCDR1, HCDR2, and a HCDR3 as set forth for Clone 58 in Table 6. In some embodiments, the variable heavy chain has a HCDR1, HCDR2, and a HCDR3 as set forth for Clone 59 in Table 6. In some embodiments, the variable heavy chain has a HCDR1, HCDR2, and a HCDR3 as set forth for Clone 60 in Table 6. In some embodiments, the variable heavy chain has a HCDR1, HCDR2, and a HCDR3 as set forth for Clone 61 in Table 6. In some embodiments, the variable heavy chain has a HCDR1, HCDR2, and a HCDR3 as set forth for Clone 62 in Table 6. In some embodiments, the variable heavy chain has a HCDR1, HCDR2, and a HCDR3 as set forth for Clone 63 in Table 6. In some embodiments, the variable heavy chain has a HCDR1, HCDR2, and a HCDR3 as set forth for Clone 64 in Table 6. In some embodiments, the variable heavy chain has a HCDR1, HCDR2, and a HCDR3 as set forth for Clone 65 in Table 6. In some embodiments, the variable heavy chain has a HCDR1, HCDR2, and a HCDR3 as set forth for Clone 66 in Table 6.

In some embodiments, the variable heavy chain has a HCDR1, HCDR2, and a HCDR3 as set forth for Clone 1 in Table 7. In some embodiments, the variable heavy chain has a HCDR1, HCDR2, and a HCDR3 as set forth for Clone 2 in Table 7. In some embodiments, the variable heavy chain has a HCDR1, HCDR2, and a HCDR3 as set forth for Clone 3 in Table 7. In some embodiments, the variable heavy chain has a HCDR1, HCDR2, and a HCDR3 as set forth for Clone 4 in Table 7. In some embodiments, the variable heavy chain has a HCDR1, HCDR2, and a HCDR3 as set forth for Clone 5 in Table 7. In some embodiments, the variable heavy chain has a HCDR1, HCDR2, and a HCDR3 as set forth for Clone 6 in Table 7. In some embodiments, the variable heavy chain has a HCDR1, HCDR2, and a HCDR3 as set forth for Clone 7 in Table 7. In some embodiments, the variable heavy chain has a HCDR1, HCDR2, and a HCDR3 as set forth for Clone 8 in Table 7. In some embodiments, the variable heavy chain has a HCDR1, HCDR2, and a HCDR3 as set forth for Clone 9 in Table 7. In some embodiments, the variable heavy chain has a HCDR1, HCDR2, and a HCDR3 as set forth for Clone 10 in Table 7. In some embodiments, the variable heavy chain has a HCDR1, HCDR2, and a HCDR3 as set forth for Clone 11 in Table 7. In some embodiments, the variable heavy chain has a HCDR1, HCDR2, and a HCDR3 as set forth for Clone 12 in Table 7. In some embodiments, the variable heavy chain has a HCDR1, HCDR2, and a HCDR3 as set forth for Clone 13 in Table 7. In some embodiments, the variable heavy chain has a HCDR1, HCDR2, and a HCDR3 as set forth for Clone 14 in Table 7. In some embodiments, the variable heavy chain has a HCDR1, HCDR2, and a HCDR3 as set forth for Clone 15 in Table 7. In some embodiments, the variable heavy chain has a HCDR1, HCDR2, and a HCDR3 as set forth for Clone 16 in Table 7. In some embodiments, the variable heavy chain has a HCDR1, HCDR2, and a HCDR3 as set forth for Clone 17 in Table 7. In some embodiments, the variable heavy chain has a HCDR1, HCDR2, and a HCDR3 as set forth for Clone 1 in Table 7. In some embodiments, the variable heavy chain has a HCDR1, HCDR2, and a HCDR3 as set forth for Clone 18 in Table 7. In some embodiments, the variable heavy chain has a HCDR1, HCDR2, and a HCDR3 as set forth for Clone 19 in Table 7. In some embodiments, the variable heavy chain has a HCDR1, HCDR2, and a HCDR3 as set forth for Clone 20 in Table 7. In some embodiments, the variable heavy chain has a HCDR1, HCDR2, and a HCDR3 as set forth for Clone 21 in Table 7. In some embodiments, the variable heavy chain has a HCDR1, HCDR2, and a HCDR3 as set forth for Clone 22 in Table 7. In some embodiments, the variable heavy chain has a HCDR1, HCDR2, and a HCDR3 as set forth for Clone 23 in Table 7. In some embodiments, the variable heavy chain has a HCDR1, HCDR2, and a HCDR3 as set forth for Clone 24 in Table 7. In some embodiments, the variable heavy chain has a HCDR1, HCDR2, and a HCDR3 as set forth for Clone 25 in Table 7. In some embodiments, the variable heavy chain has a HCDR1, HCDR2, and a HCDR3 as set forth for Clone 26 in Table 7. In some embodiments, the variable heavy chain has a HCDR1, HCDR2, and a HCDR3 as set forth for Clone 27 in Table 7. In some embodiments, the variable heavy chain has a HCDR1, HCDR2, and a HCDR3 as set forth for Clone 28 in Table 7. In some embodiments, the variable heavy chain has a HCDR1, HCDR2, and a HCDR3 as set forth for Clone 29 in Table 7. In some embodiments, the variable heavy chain has a HCDR1, HCDR2, and a HCDR3 as set forth for Clone 30 in Table 7. In some embodiments, the variable heavy chain has a HCDR1, HCDR2, and a HCDR3 as set forth for Clone 31 in Table 7. In some embodiments, the variable heavy chain has a HCDR1, HCDR2, and a HCDR3 as set forth for Clone 32 in Table 7. In some embodiments, the variable heavy chain has a HCDR1, HCDR2, and a HCDR3 as set forth for Clone 33 in Table 7. In some embodiments, the variable heavy chain has a HCDR1, HCDR2, and a HCDR3 as set forth for Clone 34 in Table 7. In some embodiments, the variable heavy chain has a HCDR1, HCDR2, and a HCDR3 as set forth for Clone 35 in Table 7. In some embodiments, the variable heavy chain has a HCDR1, HCDR2, and a HCDR3 as set forth for Clone 36 in Table 7. In some embodiments, the variable heavy chain has a HCDR1, HCDR2, and a HCDR3 as set forth for Clone 37 in Table 7. In some embodiments, the variable heavy chain has a HCDR1, HCDR2, and a HCDR3 as set forth for Clone 38 in Table 7. In some embodiments, the variable heavy chain has a HCDR1, HCDR2, and a HCDR3 as set forth for Clone 39 in Table 7. In some embodiments, the variable heavy chain has a HCDR1, HCDR2, and a HCDR3 as set forth for Clone 40 in Table 7. In some embodiments, the variable heavy chain has a HCDR1, HCDR2, and a HCDR3 as set forth for Clone 41 in Table 7. In some embodiments, the variable heavy chain has a HCDR1, HCDR2, and a HCDR3 as set forth for Clone 42 in Table 7. In some embodiments, the variable heavy chain has a HCDR1, HCDR2, and a HCDR3 as set forth for Clone 43 in Table 7. In some embodiments, the variable heavy chain has a HCDR1, HCDR2, and a HCDR3 as set forth for Clone 44 in Table 7. In some embodiments, the variable heavy chain has a HCDR1, HCDR2, and a HCDR3 as set forth for Clone 45 in Table 7. In some embodiments, the variable heavy chain has a HCDR1, HCDR2, and a HCDR3 as set forth for Clone 46 in Table 7. In some embodiments, the variable heavy chain has a HCDR1, HCDR2, and a HCDR3 as set forth for Clone 47 in Table 7. In some embodiments, the variable heavy chain has a HCDR1, HCDR2, and a HCDR3 as set forth for Clone 48 in Table 7. In some embodiments, the variable heavy chain has a HCDR1, HCDR2, and a HCDR3 as set forth for Clone 49 in Table 7. In some embodiments, the variable heavy chain has a HCDR1, HCDR2, and a HCDR3 as set forth for Clone 50 in Table 7. In some embodiments, the variable heavy chain has a HCDR1, HCDR2, and a HCDR3 as set forth for Clone 51 in Table 7. In some embodiments, the variable heavy chain has a HCDR1, HCDR2, and a HCDR3 as set forth for Clone 52 in Table 7. In some embodiments, the variable heavy chain has a HCDR1, HCDR2, and a HCDR3 as set forth for Clone 53 in Table 7. In some embodiments, the variable heavy chain has a HCDR1, HCDR2, and a HCDR3 as set forth for Clone 54 in Table 7. In some embodiments, the variable heavy chain has a HCDR1, HCDR2, and a HCDR3 as set forth for Clone 55 in Table 7. In some embodiments, the variable heavy chain has a HCDR1, HCDR2, and a HCDR3 as set forth for Clone 56 in Table 7. In some embodiments, the variable heavy chain has a HCDR1, HCDR2, and a HCDR3 as set forth for Clone 57 in Table 7. In some embodiments, the variable heavy chain has a HCDR1, HCDR2, and a HCDR3 as set forth for Clone 58 in Table 7. In some embodiments, the variable heavy chain has a HCDR1, HCDR2, and a HCDR3 as set forth for Clone 59 in Table 7. In some embodiments, the variable heavy chain has a HCDR1, HCDR2, and a HCDR3 as set forth for Clone 60 in Table 7. In some embodiments, the variable heavy chain has a HCDR1, HCDR2, and a HCDR3 as set forth for Clone 61 in Table 7. In some embodiments, the variable heavy chain has a HCDR1, HCDR2, and a HCDR3 as set forth for Clone 62 in Table 7. In some embodiments, the variable heavy chain has a HCDR1, HCDR2, and a HCDR3 as set forth for Clone 63 in Table 7. In some embodiments, the variable heavy chain has a HCDR1, HCDR2, and a HCDR3 as set forth for Clone 64 in Table 7. In some embodiments, the variable heavy chain has a HCDR1, HCDR2, and a HCDR3 as set forth for Clone 65 in Table 7. In some embodiments, the variable heavy chain has a HCDR1, HCDR2, and a HCDR3 as set forth for Clone 66 in Table 7. In some embodiments, the variable heavy chain has a HCDR1, HCDR2, and a HCDR3 as set forth for Clone 67 in Table 7. In some embodiments, the variable heavy chain has a HCDR1, HCDR2, and a HCDR3 as set forth for Clone 68 in Table 7. In some embodiments, the variable heavy chain has a HCDR1, HCDR2, and a HCDR3 as set forth for Clone 69 in Table 7. In some embodiments, the variable heavy chain has a HCDR1, HCDR2, and a HCDR3 as set forth for Clone 70 in Table 7. In some embodiments, the variable heavy chain has a HCDR1, HCDR2, and a HCDR3 as set forth for Clone 71 in Table 7. In some embodiments, the variable heavy chain has a HCDR1, HCDR2, and a HCDR3 as set forth for Clone 72 in Table 7. In some embodiments, the variable heavy chain has a HCDR1, HCDR2, and a HCDR3 as set forth for Clone 73 in Table 7. In some embodiments, the variable heavy chain has a HCDR1, HCDR2, and a HCDR3 as set forth for Clone 74 in Table 7. In some embodiments, the variable heavy chain has a HCDR1, HCDR2, and a HCDR3 as set forth for Clone 75 in Table 7. In some embodiments, the variable heavy chain has a HCDR1, HCDR2, and a HCDR3 as set forth for Clone 76 in Table 7. In some embodiments, the variable heavy chain has a HCDR1, HCDR2, and a HCDR3 as set forth for Clone 77 in Table 7. In some embodiments, the variable heavy chain has a HCDR1, HCDR2, and a HCDR3 as set forth for Clone 78 in Table 7. In some embodiments, the variable heavy chain has a HCDR1, HCDR2, and a HCDR3 as set forth for Clone 79 in Table 7. In some embodiments, the variable heavy chain has a HCDR1, HCDR2, and a HCDR3 as set forth for Clone 80 in Table 7. In some embodiments, the variable heavy chain has a HCDR1, HCDR2, and a HCDR3 as set forth for Clone 81 in Table 7. In some embodiments, the variable heavy chain has a HCDR1, HCDR2, and a HCDR3 as set forth for Clone 82 in Table 7. In some embodiments, the variable heavy chain has a HCDR1, HCDR2, and a HCDR3 as set forth for Clone 83 in Table 7. In some embodiments, the variable heavy chain has a HCDR1, HCDR2, and a HCDR3 as set forth for Clone 84 in Table 7.

In some embodiments, the variable heavy chain has a HCDR1, HCDR2, and a HCDR3 as set forth for MIAB128 in Table 9. In some embodiments, the variable heavy chain has a HCDR1, HCDR2, and a HCDR3 as set forth for MIAB128A in Table 9. In some embodiments, the variable heavy chain has a HCDR1, HCDR2, and a HCDR3 as set forth for MIAB129 in Table 9. In some embodiments, the variable heavy chain has a HCDR1, HCDR2, and a HCDR3 as set forth for MIAB130 in Table 9. In some embodiments, the variable heavy chain has a HCDR1, HCDR2, and a HCDR3 as set forth for MIAB131 in Table 9. In some embodiments, the variable heavy chain has a HCDR1, HCDR2, and a HCDR3 as set forth for MIAB132 in Table 9. In some embodiments, the variable heavy chain has a HCDR1, HCDR2, and a HCDR3 as set forth for MIAB133 in Table 9. In some embodiments, the variable heavy chain has a HCDR1, HCDR2, and a HCDR3 as set forth for MIAB134 in Table 9. In some embodiments, the variable heavy chain has a HCDR1, HCDR2, and a HCDR3 as set forth for MIAB135 in Table 9. In some embodiments, the variable heavy chain has a HCDR1, HCDR2, and a HCDR3 as set forth for MIAB136 in Table 9. In some embodiments, the variable heavy chain has a HCDR1, HCDR2, and a HCDR3 as set forth for MIAB137 in Table 9. In some embodiments, the variable heavy chain has a HCDR1, HCDR2, and a HCDR3 as set forth for MIAB138 in Table 9. In some embodiments, the variable heavy chain has a HCDR1, HCDR2, and a HCDR3 as set forth for MIAB139 in Table 9. In some embodiments, the variable heavy chain has a HCDR1, HCDR2, and a HCDR3 as set forth for MIAB140 in Table 9. In some embodiments, the variable heavy chain has a HCDR1, HCDR2, and a HCDR3 as set forth for MIAB141 in Table 9. In some embodiments, the variable heavy chain has a HCDR1, HCDR2, and a HCDR3 as set forth for MIAB142 in Table 9. In some embodiments, the variable heavy chain has a HCDR1, HCDR2, and a HCDR3 as set forth for MIAB143 in Table 9. In some embodiments, the variable heavy chain has a HCDR1, HCDR2, and a HCDR3 as set forth for MIAB144 in Table 9. In some embodiments, the variable heavy chain has a HCDR1, HCDR2, and a HCDR3 as set forth for MIAB145 in Table 9. In some embodiments, the variable heavy chain has a HCDR1, HCDR2, and a HCDR3 as set forth for MIAB146 in Table 9. In some embodiments, the variable heavy chain has a HCDR1, HCDR2, and a HCDR3 as set forth for MIAB147 in Table 9. In some embodiments, the variable heavy chain has a HCDR1, HCDR2, and a HCDR3 as set forth for MIAB148 in Table 9. In some embodiments, the variable heavy chain has a HCDR1, HCDR2, and a HCDR3 as set forth for MIAB149 in Table 9. In some embodiments, the variable heavy chain has a HCDR1, HCDR2, and a HCDR3 as set forth for MIAB150 in Table 9. In some embodiments, the variable heavy chain has a HCDR1, HCDR2, and a HCDR3 as set forth for MIAB151 in Table 9. In some embodiments, the variable heavy chain has a HCDR1, HCDR2, and a HCDR3 as set forth for MIAB152 in Table 9. In some embodiments, the variable heavy chain has a HCDR1, HCDR2, and a HCDR3 as set forth for MIAB153 in Table 9. In some embodiments, the variable heavy chain has a HCDR1, HCDR2, and a HCDR3 as set forth for MIAB154 in Table 9. In some embodiments, the variable heavy chain has a HCDR1, HCDR2, and a HCDR3 as set forth for MIAB155 in Table 9. In some embodiments, the variable heavy chain has a HCDR1, HCDR2, and a HCDR3 as set forth for MIAB156 in Table 9. In some embodiments, the variable heavy chain has a HCDR1, HCDR2, and a HCDR3 as set forth for MIAB157 in Table 9. In some embodiments, the variable heavy chain has a HCDR1, HCDR2, and a HCDR3 as set forth for MIAB158 in Table 9. In some embodiments, the variable heavy chain has a HCDR1, HCDR2, and a HCDR3 as set forth for MIAB159 in Table 9. In some embodiments, the variable heavy chain has a HCDR1, HCDR2, and a HCDR3 as set forth for MIAB160 in Table 9. In some embodiments, the variable heavy chain has a HCDR1, HCDR2, and a HCDR3 as set forth for MIAB161 in Table 9. In some embodiments, the variable heavy chain has a HCDR1, HCDR2, and a HCDR3 as set forth for MIAB162 in Table 9. In some embodiments, the variable heavy chain has a HCDR1, HCDR2, and a HCDR3 as set forth for MIAB163 in Table 9. In some embodiments, the variable heavy chain has a HCDR1, HCDR2, and a HCDR3 as set forth for MIAB164 in Table 9. In some embodiments, the variable heavy chain has a HCDR1, HCDR2, and a HCDR3 as set forth for MIAB165 in Table 9. In some embodiments, the variable heavy chain has a HCDR1, HCDR2, and a HCDR3 as set forth for MIAB166 in Table 9. In some embodiments, the variable heavy chain has a HCDR1, HCDR2, and a HCDR3 as set forth for MIAB167 in Table 9. In some embodiments, the variable heavy chain has a HCDR1, HCDR2, and a HCDR3 as set forth for MIAB168 in Table 9. In some embodiments, the variable heavy chain has a HCDR1, HCDR2, and a HCDR3 as set forth for MIAB169 in Table 9. In some embodiments, the variable heavy chain has a HCDR1, HCDR2, and a HCDR3 as set forth for MIAB170 in Table 9. In some embodiments, the variable heavy chain has a HCDR1, HCDR2, and a HCDR3 as set forth for MIAB171 in Table 9. In some embodiments, the variable heavy chain has a HCDR1, HCDR2, and a HCDR3 as set forth for MIAB172 in Table 9. In some embodiments, the variable heavy chain has a HCDR1, HCDR2, and a HCDR3 as set forth for MIAB173 in Table 9. In some embodiments, the variable heavy chain has a HCDR1, HCDR2, and a HCDR3 as set forth for MIAB174 in Table 9. In some embodiments, the variable heavy chain has a HCDR1, HCDR2, and a HCDR3 as set forth for MIAB175 in Table 9. In some embodiments, the variable heavy chain has a HCDR1, HCDR2, and a HCDR3 as set forth for MIAB176 in Table 9. In some embodiments, the variable heavy chain has a HCDR1, HCDR2, and a HCDR3 as set forth for MIAB177 in Table 9. In some embodiments, the variable heavy chain has a HCDR1, HCDR2, and a HCDR3 as set forth for MIAB178 in Table 9. In some embodiments, the variable heavy chain has a HCDR1, HCDR2, and a HCDR3 as set forth for MIAB179 in Table 9. In some embodiments, the variable heavy chain has a HCDR1, HCDR2, and a HCDR3 as set forth for MIAB180 in Table 9. In some embodiments, the variable heavy chain has a HCDR1, HCDR2, and a HCDR3 as set forth for MIAB181 in Table 9. In some embodiments, the variable heavy chain has a HCDR1, HCDR2, and a HCDR3 as set forth for MIAB182 in Table 9. In some embodiments, the variable heavy chain has a HCDR1, HCDR2, and a HCDR3 as set forth for MIAB183 in Table 9. In some embodiments, the variable heavy chain has a HCDR1, HCDR2, and a HCDR3 as set forth for MIAB184 in Table 9. In some embodiments, the variable heavy chain has a HCDR1, HCDR2, and a HCDR3 as set forth for MIAB185 in Table 9. In some embodiments, the variable heavy chain has a HCDR1, HCDR2, and a HCDR3 as set forth for MIAB186 in Table 9. In some embodiments, the variable heavy chain has a HCDR1, HCDR2, and a HCDR3 as set forth for MIAB187 in Table 9. In some embodiments, the variable heavy chain has a HCDR1, HCDR2, and a HCDR3 as set forth for MIAB188 in Table 9. In some embodiments, the variable heavy chain has a HCDR1, HCDR2, and a HCDR3 as set forth for MIAB189 in Table 9. In some embodiments, the variable heavy chain has a HCDR1, HCDR2, and a HCDR3 as set forth for MIAB190 in Table 9. In some embodiments, the variable heavy chain has a HCDR1, HCDR2, and a HCDR3 as set forth for MIAB191 in Table 9. In some embodiments, the variable heavy chain has a HCDR1, HCDR2, and a HCDR3 as set forth for MIAB192 in Table 9. In some embodiments, the variable heavy chain has a HCDR1, HCDR2, and a HCDR3 as set forth for MIAB193 in Table 9. In some embodiments, the variable heavy chain has a HCDR1, HCDR2, and a HCDR3 as set forth for MIAB194 in Table 9. In some embodiments, the variable heavy chain has a HCDR1, HCDR2, and a HCDR3 as set forth for MIAB195 in Table 9. In some embodiments, the variable heavy chain has a HCDR1, HCDR2, and a HCDR3 as set forth for MIAB196 in Table 9. In some embodiments, the variable heavy chain has a HCDR1, HCDR2, and a HCDR3 as set forth for MIAB197 in Table 9. In some embodiments, the variable heavy chain has a HCDR1, HCDR2, and a HCDR3 as set forth for MIAB198 in Table 9.

In some embodiments, the variable heavy chain has a HCDR1, HCDR2, and a HCDR3 as set forth for PMAB1 in Table 14. In some embodiments, the variable heavy chain has a HCDR1, HCDR2, and a HCDR3 as set forth for PMAB3 in Table 14. In some embodiments, the variable heavy chain has a HCDR1, HCDR2, and a HCDR3 as set forth for PMAB4 in Table 14. In some embodiments, the variable heavy chain has a HCDR1, HCDR2, and a HCDR3 as set forth for PMAB5 in Table 14. In some embodiments, the variable heavy chain has a HCDR1, HCDR2, and a HCDR3 as set forth for PMAB6 in Table 14. In some embodiments, the variable heavy chain has a HCDR1, HCDR2, and a HCDR3 as set forth for PMAB7 in Table 14. In some embodiments, the variable heavy chain has a HCDR1, HCDR2, and a HCDR3 as set forth for PMAB8 in Table 14. In some embodiments, the variable heavy chain has a HCDR1, HCDR2, and a HCDR3 as set forth for PMAB9 in Table 14. In some embodiments, the variable heavy chain has a HCDR1, HCDR2, and a HCDR3 as set forth for PMAB1 in Table 14. In some embodiments, the variable heavy chain has a HCDR1, HCDR2, and a HCDR3 as set forth for PMAB10 in Table 14. In some embodiments, the variable heavy chain has a HCDR1, HCDR2, and a HCDR3 as set forth for PMAB11 in Table 14. In some embodiments, the variable heavy chain has a HCDR1, HCDR2, and a HCDR3 as set forth for PMAB12 in Table 14. In some embodiments, the variable heavy chain has a HCDR1, HCDR2, and a HCDR3 as set forth for PMAB13 in Table 14. In some embodiments, the variable heavy chain has a HCDR1, HCDR2, and a HCDR3 as set forth for PMAB14 in Table 14. In some embodiments, the variable heavy chain has a HCDR1, HCDR2, and a HCDR3 as set forth for PMAB15 in Table 14. In some embodiments, the variable heavy chain has a HCDR1, HCDR2, and a HCDR3 as set forth for PMAB16 in Table 14. In some embodiments, the variable heavy chain has a HCDR1, HCDR2, and a HCDR3 as set forth for PMAB1 in Table 14. In some embodiments, the variable heavy chain has a HCDR1, HCDR2, and a HCDR3 as set forth for PMAB19 in Table 14. In some embodiments, the variable heavy chain has a HCDR1, HCDR2, and a HCDR3 as set forth for PMAB20 in Table 14. In some embodiments, the variable heavy chain has a HCDR1, HCDR2, and a HCDR3 as set forth for PMAB21 in Table 14. In some embodiments, the variable heavy chain has a HCDR1, HCDR2, and a HCDR3 as set forth for PMAB22 in Table 14. In some embodiments, the variable heavy chain has a HCDR1, HCDR2, and a HCDR3 as set forth for PMAB23 in Table 14. In some embodiments, the variable heavy chain has a HCDR1, HCDR2, and a HCDR3 as set forth for PMAB24 in Table 14. In some embodiments, the variable heavy chain has a HCDR1, HCDR2, and a HCDR3 as set forth for PMAB25 in Table 14. In some embodiments, the variable heavy chain has a HCDR1, HCDR2, and a HCDR3 as set forth for PMAB26 in Table 14. In some embodiments, the variable heavy chain has a HCDR1, HCDR2, and a HCDR3 as set forth for PMAB27 in Table 14. In some embodiments, the variable heavy chain has a HCDR1, HCDR2, and a HCDR3 as set forth for PMAB28 in Table 14. In some embodiments, the variable heavy chain has a HCDR1, HCDR2, and a HCDR3 as set forth for PMAB29 in Table 14. In some embodiments, the variable heavy chain has a HCDR1, HCDR2, and a HCDR3 as set forth for PMAB30 in Table 14. In some embodiments, the variable heavy chain has a HCDR1, HCDR2, and a HCDR3 as set forth for PMAB31 in Table 14. In some embodiments, the variable heavy chain has a HCDR1, HCDR2, and a HCDR3 as set forth for PMAB32 in Table 14. In some embodiments, the variable heavy chain has a HCDR1, HCDR2, and a HCDR33 as set forth for PMAB33 in Table 14. In some embodiments, the variable heavy chain has a HCDR1, HCDR2, and a HCDR3 as set forth for PMAB34 in Table 14. In some embodiments, the variable heavy chain has a HCDR1, HCDR2, and a HCDR3 as set forth for PMAB35 in Table 14. In some embodiments, the variable heavy chain has a HCDR1, HCDR2, and a HCDR3 as set forth for PMAB36 in Table 14. In some embodiments, the variable heavy chain has a HCDR1, HCDR2, and a HCDR3 as set forth for PMAB37 in Table 14. In some embodiments, the variable heavy chain has a HCDR1, HCDR2, and a HCDR3 as set forth for PMAB38 in Table 14. In some embodiments, the variable heavy chain has a HCDR1, HCDR2, and a HCDR3 as set forth for PMAB39 in Table 14. In some embodiments, the variable heavy chain has a HCDR1, HCDR2, and a HCDR3 as set forth for PMAB40 in Table 14. In some embodiments, the variable heavy chain has a HCDR1, HCDR2, and a HCDR3 as set forth for PMAB41 in Table 14. In some embodiments, the variable heavy chain has a HCDR1, HCDR2, and a HCDR3 as set forth for PMAB42 in Table 14. In some embodiments, the variable heavy chain has a HCDR1, HCDR2, and a HCDR3 as set forth for PMAB43 in Table 14. In some embodiments, the variable heavy chain has a HCDR1, HCDR2, and a HCDR3 as set forth for PMAB44 in Table 14. In some embodiments, the variable heavy chain has a HCDR1, HCDR2, and a HCDR3 as set forth for PMAB45 in Table 14. In some embodiments, the variable heavy chain has a HCDR1, HCDR2, and a HCDR3 as set forth for PMAB46 in Table 14. In some embodiments, the variable heavy chain has a HCDR1, HCDR2, and a HCDR3 as set forth for PMAB47 in Table 14. In some embodiments, the variable heavy chain has a HCDR1, HCDR2, and a HCDR3 as set forth for PMAB48 in Table 14. In some embodiments, the variable heavy chain has a HCDR1, HCDR2, and a HCDR3 as set forth for PMAB49 in Table 14. In some embodiments, the variable heavy chain has a HCDR1, HCDR2, and a HCDR3 as set forth for PMAB50 in Table 14. In some embodiments, the variable heavy chain has a HCDR1, HCDR2, and a HCDR3 as set forth for PMAB51 in Table 14. In some embodiments, the variable heavy chain has a HCDR1, HCDR2, and a HCDR3 as set forth for PMAB52 in Table 14. In some embodiments, the variable heavy chain has a HCDR1, HCDR2, and a HCDR3 as set forth for PMAB53 in Table 14. In some embodiments, the variable heavy chain has a HCDR1, HCDR2, and a HCDR3 as set forth for PMAB55 in Table 14.

In some embodiments, the variable heavy chain has a HCDR1, HCDR2, and a HCDR3 as set forth for MIAB212 in Table 15. In some embodiments, the variable heavy chain has a HCDR1, HCDR2, and a HCDR3 as set forth for MIAB213 in Table 15. In some embodiments, the variable heavy chain has a HCDR1, HCDR2, and a HCDR3 as set forth for MIAB214 in Table 15.

In some embodiments, the variable light chain has a LCDR1, LCDR2, and a LCDR3 as set forth for Clone 1 in Table 6. In some embodiments, the variable heavy chain has a LCDR1, LCDR2, and a LCDR3 as set forth for Clone 2 in Table 6. In some embodiments, the variable heavy chain has a LCDR1, LCDR2, and a LCDR3 as set forth for Clone 3 in Table 6. In some embodiments, the variable heavy chain has a LCDR1, LCDR2, and a LCDR3 as set forth for Clone 4 in Table 6. In some embodiments, the variable heavy chain has a LCDR1, LCDR2, and a LCDR3 as set forth for Clone 5 in Table 6. In some embodiments, the variable heavy chain has a LCDR1, LCDR2, and a LCDR3 as set forth for Clone 6 in Table 6. In some embodiments, the variable heavy chain has a LCDR1, LCDR2, and a LCDR3 as set forth for Clone 7 in Table 6. In some embodiments, the variable heavy chain has a LCDR1, LCDR2, and a LCDR3 as set forth for Clone 8 in Table 6. In some embodiments, the variable heavy chain has a LCDR1, LCDR2, and a LCDR3 as set forth for Clone 9 in Table 6. In some embodiments, the variable heavy chain has a LCDR1, LCDR2, and a LCDR3 as set forth for Clone 10 in Table 6. In some embodiments, the variable heavy chain has a LCDR1, LCDR2, and a LCDR3 as set forth for Clone 11 in Table 6. In some embodiments, the variable heavy chain has a LCDR1, LCDR2, and a LCDR3 as set forth for Clone 12 in Table 6. In some embodiments, the variable heavy chain has a LCDR1, LCDR2, and a LCDR3 as set forth for Clone 13 in Table 6. In some embodiments, the variable heavy chain has a LCDR1, LCDR2, and a LCDR3 as set forth for Clone 14 in Table 6. In some embodiments, the variable heavy chain has a LCDR1, LCDR2, and a LCDR3 as set forth for Clone 15 in Table 6. In some embodiments, the variable heavy chain has a LCDR1, LCDR2, and a LCDR3 as set forth for Clone 16 in Table 6. In some embodiments, the variable heavy chain has a LCDR1, LCDR2, and a LCDR3 as set forth for Clone 17 in Table 6. In some embodiments, the variable heavy chain has a LCDR1, LCDR2, and a LCDR3 as set forth for Clone 1 in Table 6. In some embodiments, the variable heavy chain has a LCDR1, LCDR2, and a LCDR3 as set forth for Clone 18 in Table 6. In some embodiments, the variable heavy chain has a LCDR1, LCDR2, and a LCDR3 as set forth for Clone 19 in Table 6. In some embodiments, the variable heavy chain has a LCDR1, LCDR2, and a LCDR3 as set forth for Clone 20 in Table 6. In some embodiments, the variable heavy chain has a LCDR1, LCDR2, and a LCDR3 as set forth for Clone 21 in Table 6. In some embodiments, the variable heavy chain has a LCDR1, LCDR2, and a LCDR3 as set forth for Clone 22 in Table 6. In some embodiments, the variable heavy chain has a LCDR1, LCDR2, and a LCDR3 as set forth for Clone 23 in Table 6. In some embodiments, the variable heavy chain has a LCDR1, LCDR2, and a LCDR3 as set forth for Clone 24 in Table 6. In some embodiments, the variable heavy chain has a LCDR1, LCDR2, and a LCDR3 as set forth for Clone 25 in Table 6. In some embodiments, the variable heavy chain has a LCDR1, LCDR2, and a LCDR3 as set forth for Clone 26 in Table 6. In some embodiments, the variable heavy chain has a LCDR1, LCDR2, and a LCDR3 as set forth for Clone 27 in Table 6. In some embodiments, the variable heavy chain has a LCDR1, LCDR2, and a LCDR3 as set forth for Clone 28 in Table 6. In some embodiments, the variable heavy chain has a LCDR1, LCDR2, and a LCDR3 as set forth for Clone 29 in Table 6. In some embodiments, the variable heavy chain has a LCDR1, LCDR2, and a LCDR3 as set forth for Clone 30 in Table 6. In some embodiments, the variable heavy chain has a LCDR1, LCDR2, and a LCDR3 as set forth for Clone 31 in Table 6. In some embodiments, the variable heavy chain has a LCDR1, LCDR2, and a LCDR3 as set forth for Clone 32 in Table 6. In some embodiments, the variable heavy chain has a LCDR1, LCDR2, and a LCDR3 as set forth for Clone 33 in Table 6. In some embodiments, the variable heavy chain has a LCDR1, LCDR2, and a LCDR3 as set forth for Clone 34 in Table 6. In some embodiments, the variable heavy chain has a LCDR1, LCDR2, and a LCDR3 as set forth for Clone 35 in Table 6. In some embodiments, the variable heavy chain has a LCDR1, LCDR2, and a LCDR3 as set forth for Clone 36 in Table 6. In some embodiments, the variable heavy chain has a LCDR1, LCDR2, and a LCDR3 as set forth for Clone 37 in Table 6. In some embodiments, the variable heavy chain has a LCDR1, LCDR2, and a LCDR3 as set forth for Clone 38 in Table 6. In some embodiments, the variable heavy chain has a LCDR1, LCDR2, and a LCDR3 as set forth for Clone 39 in Table 6. In some embodiments, the variable heavy chain has a LCDR1, LCDR2, and a LCDR3 as set forth for Clone 40 in Table 6. In some embodiments, the variable heavy chain has a LCDR1, LCDR2, and a LCDR3 as set forth for Clone 41 in Table 6. In some embodiments, the variable heavy chain has a LCDR1, LCDR2, and a LCDR3 as set forth for Clone 42 in Table 6. In some embodiments, the variable heavy chain has a LCDR1, LCDR2, and a LCDR3 as set forth for Clone 43 in Table 6. In some embodiments, the variable heavy chain has a LCDR1, LCDR2, and a LCDR3 as set forth for Clone 44 in Table 6. In some embodiments, the variable heavy chain has a LCDR1, LCDR2, and a LCDR3 as set forth for Clone 45 in Table 6. In some embodiments, the variable heavy chain has a LCDR1, LCDR2, and a LCDR3 as set forth for Clone 46 in Table 6. In some embodiments, the variable heavy chain has a LCDR1, LCDR2, and a LCDR3 as set forth for Clone 47 in Table 6. In some embodiments, the variable heavy chain has a LCDR1, LCDR2, and a LCDR3 as set forth for Clone 48 in Table 6. In some embodiments, the variable heavy chain has a LCDR1, LCDR2, and a LCDR3 as set forth for Clone 49 in Table 6. In some embodiments, the variable heavy chain has a LCDR1, LCDR2, and a LCDR3 as set forth for Clone 50 in Table 6. In some embodiments, the variable heavy chain has a LCDR1, LCDR2, and a LCDR3 as set forth for Clone 51 in Table 6. In some embodiments, the variable heavy chain has a LCDR1, LCDR2, and a LCDR3 as set forth for Clone 52 in Table 6. In some embodiments, the variable heavy chain has a LCDR1, LCDR2, and a LCDR3 as set forth for Clone 53 in Table 6. In some embodiments, the variable heavy chain has a LCDR1, LCDR2, and a LCDR3 as set forth for Clone 54 in Table 6. In some embodiments, the variable heavy chain has a LCDR1, LCDR2, and a LCDR3 as set forth for Clone 55 in Table 6. In some embodiments, the variable heavy chain has a LCDR1, LCDR2, and a LCDR3 as set forth for Clone 56 in Table 6. In some embodiments, the variable heavy chain has a LCDR1, LCDR2, and a LCDR3 as set forth for Clone 57 in Table 6. In some embodiments, the variable heavy chain has a LCDR1, LCDR2, and a LCDR3 as set forth for Clone 58 in Table 6. In some embodiments, the variable heavy chain has a LCDR1, LCDR2, and a LCDR3 as set forth for Clone 59 in Table 6. In some embodiments, the variable heavy chain has a LCDR1, LCDR2, and a LCDR3 as set forth for Clone 60 in Table 6. In some embodiments, the variable heavy chain has a LCDR1, LCDR2, and a LCDR3 as set forth for Clone 61 in Table 6. In some embodiments, the variable heavy chain has a LCDR1, LCDR2, and a LCDR3 as set forth for Clone 62 in Table 6. In some embodiments, the variable heavy chain has a LCDR1, LCDR2, and a LCDR3 as set forth for Clone 63 in Table 6. In some embodiments, the variable heavy chain has a LCDR1, LCDR2, and a LCDR3 as set forth for Clone 64 in Table 6. In some embodiments, the variable heavy chain has a LCDR1, LCDR2, and a LCDR3 as set forth for Clone 65 in Table 6. In some embodiments, the variable heavy chain has a LCDR1, LCDR2, and a LCDR3 as set forth for Clone 66 in Table 6.

In some embodiments, the variable heavy chain has a LCDR1, LCDR2, and a LCDR3 as set forth for Clone 1 in Table 7. In some embodiments, the variable heavy chain has a LCDR1, LCDR2, and a LCDR3 as set forth for Clone 2 in Table 7. In some embodiments, the variable heavy chain has a LCDR1, LCDR2, and a LCDR3 as set forth for Clone 3 in Table 7. In some embodiments, the variable heavy chain has a LCDR1, LCDR2, and a LCDR3 as set forth for Clone 4 in Table 7. In some embodiments, the variable heavy chain has a LCDR1, LCDR2, and a LCDR3 as set forth for Clone 5 in Table 7. In some embodiments, the variable heavy chain has a LCDR1, LCDR2, and a LCDR3 as set forth for Clone 6 in Table 7. In some embodiments, the variable heavy chain has a LCDR1, LCDR2, and a LCDR3 as set forth for Clone 7 in Table 7. In some embodiments, the variable heavy chain has a LCDR1, LCDR2, and a LCDR3 as set forth for Clone 8 in Table 7. In some embodiments, the variable heavy chain has a LCDR1, LCDR2, and a LCDR3 as set forth for Clone 9 in Table 7. In some embodiments, the variable heavy chain has a LCDR1, LCDR2, and a LCDR3 as set forth for Clone 10 in Table 7. In some embodiments, the variable heavy chain has a LCDR1, LCDR2, and a LCDR3 as set forth for Clone 11 in Table 7. In some embodiments, the variable heavy chain has a LCDR1, LCDR2, and a LCDR3 as set forth for Clone 12 in Table 7. In some embodiments, the variable heavy chain has a LCDR1, LCDR2, and a LCDR3 as set forth for Clone 13 in Table 7. In some embodiments, the variable heavy chain has a LCDR1, LCDR2, and a LCDR3 as set forth for Clone 14 in Table 7. In some embodiments, the variable heavy chain has a LCDR1, LCDR2, and a LCDR3 as set forth for Clone 15 in Table 7. In some embodiments, the variable heavy chain has a LCDR1, LCDR2, and a LCDR3 as set forth for Clone 16 in Table 7. In some embodiments, the variable heavy chain has a LCDR1, LCDR2, and a LCDR3 as set forth for Clone 17 in Table 7. In some embodiments, the variable heavy chain has a LCDR1, LCDR2, and a LCDR3 as set forth for Clone 1 in Table 7. In some embodiments, the variable heavy chain has a LCDR1, LCDR2, and a LCDR3 as set forth for Clone 18 in Table 7. In some embodiments, the variable heavy chain has a LCDR1, LCDR2, and a LCDR3 as set forth for Clone 19 in Table 7. In some embodiments, the variable heavy chain has a LCDR1, LCDR2, and a LCDR3 as set forth for Clone 20 in Table 7. In some embodiments, the variable heavy chain has a LCDR1, LCDR2, and a LCDR3 as set forth for Clone 21 in Table 7. In some embodiments, the variable heavy chain has a LCDR1, LCDR2, and a LCDR3 as set forth for Clone 22 in Table 7. In some embodiments, the variable heavy chain has a LCDR1, LCDR2, and a LCDR3 as set forth for Clone 23 in Table 7. In some embodiments, the variable heavy chain has a LCDR1, LCDR2, and a LCDR3 as set forth for Clone 24 in Table 7. In some embodiments, the variable heavy chain has a LCDR1, LCDR2, and a LCDR3 as set forth for Clone 25 in Table 7. In some embodiments, the variable heavy chain has a LCDR1, LCDR2, and a LCDR3 as set forth for Clone 26 in Table 7. In some embodiments, the variable heavy chain has a LCDR1, LCDR2, and a LCDR3 as set forth for Clone 27 in Table 7. In some embodiments, the variable heavy chain has a LCDR1, LCDR2, and a LCDR3 as set forth for Clone 28 in Table 7. In some embodiments, the variable heavy chain has a LCDR1, LCDR2, and a LCDR3 as set forth for Clone 29 in Table 7. In some embodiments, the variable heavy chain has a LCDR1, LCDR2, and a LCDR3 as set forth for Clone 30 in Table 7. In some embodiments, the variable heavy chain has a LCDR1, LCDR2, and a LCDR3 as set forth for Clone 31 in Table 7. In some embodiments, the variable heavy chain has a LCDR1, LCDR2, and a LCDR3 as set forth for Clone 32 in Table 7. In some embodiments, the variable heavy chain has a LCDR1, LCDR2, and a LCDR3 as set forth for Clone 33 in Table 7. In some embodiments, the variable heavy chain has a LCDR1, LCDR2, and a LCDR3 as set forth for Clone 34 in Table 7. In some embodiments, the variable heavy chain has a LCDR1, LCDR2, and a LCDR3 as set forth for Clone 35 in Table 7. In some embodiments, the variable heavy chain has a LCDR1, LCDR2, and a LCDR3 as set forth for Clone 36 in Table 7. In some embodiments, the variable heavy chain has a LCDR1, LCDR2, and a LCDR3 as set forth for Clone 37 in Table 7. In some embodiments, the variable heavy chain has a LCDR1, LCDR2, and a LCDR3 as set forth for Clone 38 in Table 7. In some embodiments, the variable heavy chain has a LCDR1, LCDR2, and a LCDR3 as set forth for Clone 39 in Table 7. In some embodiments, the variable heavy chain has a LCDR1, LCDR2, and a LCDR3 as set forth for Clone 40 in Table 7. In some embodiments, the variable heavy chain has a LCDR1, LCDR2, and a LCDR3 as set forth for Clone 41 in Table 7. In some embodiments, the variable heavy chain has a LCDR1, LCDR2, and a LCDR3 as set forth for Clone 42 in Table 7. In some embodiments, the variable heavy chain has a LCDR1, LCDR2, and a LCDR3 as set forth for Clone 43 in Table 7. In some embodiments, the variable heavy chain has a LCDR1, LCDR2, and a LCDR3 as set forth for Clone 44 in Table 7. In some embodiments, the variable heavy chain has a LCDR1, LCDR2, and a LCDR3 as set forth for Clone 45 in Table 7. In some embodiments, the variable heavy chain has a LCDR1, LCDR2, and a LCDR3 as set forth for Clone 46 in Table 7. In some embodiments, the variable heavy chain has a LCDR1, LCDR2, and a LCDR3 as set forth for Clone 47 in Table 7. In some embodiments, the variable heavy chain has a LCDR1, LCDR2, and a LCDR3 as set forth for Clone 48 in Table 7. In some embodiments, the variable heavy chain has a LCDR1, LCDR2, and a LCDR3 as set forth for Clone 49 in Table 7. In some embodiments, the variable heavy chain has a LCDR1, LCDR2, and a LCDR3 as set forth for Clone 50 in Table 7. In some embodiments, the variable heavy chain has a LCDR1, LCDR2, and a LCDR3 as set forth for Clone 51 in Table 7. In some embodiments, the variable heavy chain has a LCDR1, LCDR2, and a LCDR3 as set forth for Clone 52 in Table 7. In some embodiments, the variable heavy chain has a LCDR1, LCDR2, and a LCDR3 as set forth for Clone 53 in Table 7. In some embodiments, the variable heavy chain has a LCDR1, LCDR2, and a LCDR3 as set forth for Clone 54 in Table 7. In some embodiments, the variable heavy chain has a LCDR1, LCDR2, and a LCDR3 as set forth for Clone 55 in Table 7. In some embodiments, the variable heavy chain has a LCDR1, LCDR2, and a LCDR3 as set forth for Clone 56 in Table 7. In some embodiments, the variable heavy chain has a LCDR1, LCDR2, and a LCDR3 as set forth for Clone 57 in Table 7. In some embodiments, the variable heavy chain has a LCDR1, LCDR2, and a LCDR3 as set forth for Clone 58 in Table 7. In some embodiments, the variable heavy chain has a LCDR1, LCDR2, and a LCDR3 as set forth for Clone 59 in Table 7. In some embodiments, the variable heavy chain has a LCDR1, LCDR2, and a LCDR3 as set forth for Clone 60 in Table 7. In some embodiments, the variable heavy chain has a LCDR1, LCDR2, and a LCDR3 as set forth for Clone 61 in Table 7. In some embodiments, the variable heavy chain has a LCDR1, LCDR2, and a LCDR3 as set forth for Clone 62 in Table 7. In some embodiments, the variable heavy chain has a LCDR1, LCDR2, and a LCDR3 as set forth for Clone 63 in Table 7. In some embodiments, the variable heavy chain has a LCDR1, LCDR2, and a LCDR3 as set forth for Clone 64 in Table 7. In some embodiments, the variable heavy chain has a LCDR1, LCDR2, and a LCDR3 as set forth for Clone 65 in Table 7. In some embodiments, the variable heavy chain has a LCDR1, LCDR2, and a LCDR3 as set forth for Clone 66 in Table 7. In some embodiments, the variable heavy chain has a LCDR1, LCDR2, and a LCDR3 as set forth for Clone 67 in Table 7. In some embodiments, the variable heavy chain has a LCDR1, LCDR2, and a LCDR3 as set forth for Clone 68 in Table 7. In some embodiments, the variable heavy chain has a LCDR1, LCDR2, and a LCDR3 as set forth for Clone 69 in Table 7. In some embodiments, the variable heavy chain has a LCDR1, LCDR2, and a LCDR3 as set forth for Clone 70 in Table 7. In some embodiments, the variable heavy chain has a LCDR1, LCDR2, and a LCDR3 as set forth for Clone 71 in Table 7. In some embodiments, the variable heavy chain has a LCDR1, LCDR2, and a LCDR3 as set forth for Clone 72 in Table 7. In some embodiments, the variable heavy chain has a LCDR1, LCDR2, and a LCDR3 as set forth for Clone 73 in Table 7. In some embodiments, the variable heavy chain has a LCDR1, LCDR2, and a LCDR3 as set forth for Clone 74 in Table 7. In some embodiments, the variable heavy chain has a LCDR1, LCDR2, and a LCDR3 as set forth for Clone 75 in Table 7. In some embodiments, the variable heavy chain has a LCDR1, LCDR2, and a LCDR3 as set forth for Clone 76 in Table 7. In some embodiments, the variable heavy chain has a LCDR1, LCDR2, and a LCDR3 as set forth for Clone 77 in Table 7. In some embodiments, the variable heavy chain has a LCDR1, LCDR2, and a LCDR3 as set forth for Clone 78 in Table 7. In some embodiments, the variable heavy chain has a LCDR1, LCDR2, and a LCDR3 as set forth for Clone 79 in Table 7. In some embodiments, the variable heavy chain has a LCDR1, LCDR2, and a LCDR3 as set forth for Clone 80 in Table 7. In some embodiments, the variable heavy chain has a LCDR1, LCDR2, and a LCDR3 as set forth for Clone 81 in Table 7. In some embodiments, the variable heavy chain has a LCDR1, LCDR2, and a LCDR3 as set forth for Clone 82 in Table 7. In some embodiments, the variable heavy chain has a LCDR1, LCDR2, and a LCDR3 as set forth for Clone 83 in Table 7. In some embodiments, the variable light chain has a LCDR1, LCDR2, and a LCDR3 as set forth for Clone 84 in Table 7.

In some embodiments, the variable light chain has a LCDR1, LCDR2, and a LCDR3 as set forth for MIAB128 in Table 9. In some embodiments, the variable light chain has a LCDR1, LCDR2, and a LCDR3 as set forth for MIAB128A in Table 9. In some embodiments, the variable light chain has a LCDR1, LCDR2, and a LCDR3 as set forth for MIAB129 in Table 9. In some embodiments, the variable light chain has a LCDR1, LCDR2, and a LCDR3 as set forth for MIAB130 in Table 9. In some embodiments, the variable light chain has a LCDR1, LCDR2, and a LCDR3 as set forth for MIAB131 in Table 9. In some embodiments, the variable light chain has a LCDR1, LCDR2, and a LCDR3 as set forth for MIAB132 in Table 9. In some embodiments, the variable light chain has a LCDR1, LCDR2, and a LCDR3 as set forth for MIAB133 in Table 9. In some embodiments, the variable light chain has a LCDR1, LCDR2, and a LCDR3 as set forth for MIAB134 in Table 9. In some embodiments, the variable light chain has a LCDR1, LCDR2, and a LCDR3 as set forth for MIAB135 in Table 9. In some embodiments, the variable light chain has a LCDR1, LCDR2, and a LCDR3 as set forth for MIAB136 in Table 9. In some embodiments, the variable light chain has a LCDR1, LCDR2, and a LCDR3 as set forth for MIAB137 in Table 9. In some embodiments, the variable light chain has a LCDR1, LCDR2, and a LCDR3 as set forth for MIAB138 in Table 9. In some embodiments, the variable light chain has a LCDR1, LCDR2, and a LCDR3 as set forth for MIAB139 in Table 9. In some embodiments, the variable light chain has a LCDR1, LCDR2, and a LCDR3 as set forth for MIAB140 in Table 9. In some embodiments, the variable light chain has a LCDR1, LCDR2, and a LCDR3 as set forth for MIAB141 in Table 9. In some embodiments, the variable light chain has a LCDR1, LCDR2, and a LCDR3 as set forth for MIAB142 in Table 9. In some embodiments, the variable light chain has a LCDR1, LCDR2, and a LCDR3 as set forth for MIAB143 in Table 9. In some embodiments, the variable light chain has a LCDR1, LCDR2, and a LCDR3 as set forth for MIAB144 in Table 9. In some embodiments, the variable light chain has a LCDR1, LCDR2, and a LCDR3 as set forth for MIAB145 in Table 9. In some embodiments, the variable light chain has a LCDR1, LCDR2, and a LCDR3 as set forth for MIAB146 in Table 9. In some embodiments, the variable light chain has a LCDR1, LCDR2, and a LCDR3 as set forth for MIAB147 in Table 9. In some embodiments, the variable light chain has a LCDR1, LCDR2, and a LCDR3 as set forth for MIAB148 in Table 9. In some embodiments, the variable light chain has a LCDR1, LCDR2, and a LCDR3 as set forth for MIAB149 in Table 9. In some embodiments, the variable light chain has a LCDR1, LCDR2, and a LCDR3 as set forth for MIAB150 in Table 9. In some embodiments, the variable light chain has a LCDR1, LCDR2, and a LCDR3 as set forth for MIAB151 in Table 9. In some embodiments, the variable light chain has a LCDR1, LCDR2, and a LCDR3 as set forth for MIAB152 in Table 9. In some embodiments, the variable light chain has a LCDR1, LCDR2, and a LCDR3 as set forth for MIAB153 in Table 9. In some embodiments, the variable light chain has a LCDR1, LCDR2, and a LCDR3 as set forth for MIAB154 in Table 9. In some embodiments, the variable light chain has a LCDR1, LCDR2, and a LCDR3 as set forth for MIAB155 in Table 9. In some embodiments, the variable light chain has a LCDR1, LCDR2, and a LCDR3 as set forth for MIAB156 in Table 9. In some embodiments, the variable light chain has a LCDR1, LCDR2, and a LCDR3 as set forth for MIAB157 in Table 9. In some embodiments, the variable light chain has a LCDR1, LCDR2, and a LCDR3 as set forth for MIAB158 in Table 9. In some embodiments, the variable light chain has a LCDR1, LCDR2, and a LCDR3 as set forth for MIAB159 in Table 9. In some embodiments, the variable light chain has a LCDR1, LCDR2, and a LCDR3 as set forth for MIAB160 in Table 9. In some embodiments, the variable light chain has a LCDR1, LCDR2, and a LCDR3 as set forth for MIAB161 in Table 9. In some embodiments, the variable light chain has a LCDR1, LCDR2, and a LCDR3 as set forth for MIAB162 in Table 9. In some embodiments, the variable light chain has a LCDR1, LCDR2, and a LCDR3 as set forth for MIAB163 in Table 9. In some embodiments, the variable light chain has a LCDR1, LCDR2, and a LCDR3 as set forth for MIAB164 in Table 9. In some embodiments, the variable light chain has a LCDR1, LCDR2, and a LCDR3 as set forth for MIAB165 in Table 9. In some embodiments, the variable light chain has a LCDR1, LCDR2, and a LCDR3 as set forth for MIAB166 in Table 9. In some embodiments, the variable light chain has a LCDR1, LCDR2, and a LCDR3 as set forth for MIAB167 in Table 9. In some embodiments, the variable light chain has a LCDR1, LCDR2, and a LCDR3 as set forth for MIAB168 in Table 9. In some embodiments, the variable light chain has a LCDR1, LCDR2, and a LCDR3 as set forth for MIAB169 in Table 9. In some embodiments, the variable light chain has a LCDR1, LCDR2, and a LCDR3 as set forth for MIAB170 in Table 9. In some embodiments, the variable light chain has a LCDR1, LCDR2, and a LCDR3 as set forth for MIAB171 in Table 9. In some embodiments, the variable light chain has a LCDR1, LCDR2, and a LCDR3 as set forth for MIAB172 in Table 9. In some embodiments, the variable light chain has a LCDR1, LCDR2, and a LCDR3 as set forth for MIAB173 in Table 9. In some embodiments, the variable light chain has a LCDR1, LCDR2, and a LCDR3 as set forth for MIAB174 in Table 9. In some embodiments, the variable light chain has a LCDR1, LCDR2, and a LCDR3 as set forth for MIAB175 in Table 9. In some embodiments, the variable light chain has a LCDR1, LCDR2, and a LCDR3 as set forth for MIAB176 in Table 9. In some embodiments, the variable light chain has a LCDR1, LCDR2, and a LCDR3 as set forth for MIAB177 in Table 9. In some embodiments, the variable light chain has a LCDR1, LCDR2, and a LCDR3 as set forth for MIAB178 in Table 9. In some embodiments, the variable light chain has a LCDR1, LCDR2, and a LCDR3 as set forth for MIAB179 in Table 9. In some embodiments, the variable light chain has a LCDR1, LCDR2, and a LCDR3 as set forth for MIAB180 in Table 9. In some embodiments, the variable light chain has a LCDR1, LCDR2, and a LCDR3 as set forth for MIAB181 in Table 9. In some embodiments, the variable light chain has a LCDR1, LCDR2, and a LCDR3 as set forth for MIAB182 in Table 9. In some embodiments, the variable light chain has a LCDR1, LCDR2, and a LCDR3 as set forth for MIAB183 in Table 9. In some embodiments, the variable light chain has a LCDR1, LCDR2, and a LCDR3 as set forth for MIAB184 in Table 9. In some embodiments, the variable light chain has a LCDR1, LCDR2, and a LCDR3 as set forth for MIAB185 in Table 9. In some embodiments, the variable light chain has a LCDR1, LCDR2, and a LCDR3 as set forth for MIAB186 in Table 9. In some embodiments, the variable light chain has a LCDR1, LCDR2, and a LCDR3 as set forth for MIAB187 in Table 9. In some embodiments, the variable light chain has a LCDR1, LCDR2, and a LCDR3 as set forth for MIAB188 in Table 9. In some embodiments, the variable light chain has a LCDR1, LCDR2, and a LCDR3 as set forth for MIAB189 in Table 9. In some embodiments, the variable light chain has a LCDR1, LCDR2, and a LCDR3 as set forth for MIAB190 in Table 9. In some embodiments, the variable light chain has a LCDR1, LCDR2, and a LCDR3 as set forth for MIAB191 in Table 9. In some embodiments, the variable light chain has a LCDR1, LCDR2, and a LCDR3 as set forth for MIAB192 in Table 9. In some embodiments, the variable light chain has a LCDR1, LCDR2, and a LCDR3 as set forth for MIAB193 in Table 9. In some embodiments, the variable light chain has a LCDR1, LCDR2, and a LCDR3 as set forth for MIAB194 in Table 9. In some embodiments, the variable light chain has a LCDR1, LCDR2, and a LCDR3 as set forth for MIAB195 in Table 9. In some embodiments, the variable light chain has a LCDR1, LCDR2, and a LCDR3 as set forth for MIAB196 in Table 9. In some embodiments, the variable light chain has a LCDR1, LCDR2, and a LCDR3 as set forth for MIAB197 in Table 9. In some embodiments, the variable light chain has a LCDR1, LCDR2, and a LCDR3 as set forth for MIAB198 in Table 9.

In some embodiments, the variable light chain has a LCDR1, LCDR2, and a LCDR3 as set forth for PMAB1 in Table 14. In some embodiments, the variable light chain has a LCDR1, LCDR2, and a LCDR3 as set forth for PMAB3 in Table 14. In some embodiments, the variable light chain has a LCDR1, LCDR2, and a LCDR3 as set forth for PMAB4 in Table 14. In some embodiments, the variable light chain has a LCDR1, LCDR2, and a LCDR3 as set forth for PMAB5 in Table 14. In some embodiments, the variable light chain has a LCDR1, LCDR2, and a LCDR3 as set forth for PMAB6 in Table 14. In some embodiments, the variable light chain has a LCDR1, LCDR2, and a LCDR3 as set forth for PMAB7 in Table 14. In some embodiments, the variable light chain has a LCDR1, LCDR2, and a LCDR3 as set forth for PMAB8 in Table 14. In some embodiments, the variable light chain has a LCDR1, LCDR2, and a LCDR3 as set forth for PMAB9 in Table 14. In some embodiments, the variable light chain has a LCDR1, LCDR2, and a LCDR3 as set forth for PMAB1 in Table 14. In some embodiments, the variable light chain has a LCDR1, LCDR2, and a LCDR3 as set forth for PMAB10 in Table 14. In some embodiments, the variable light chain has a LCDR1, LCDR2, and a LCDR3 as set forth for PMAB11 in Table 14. In some embodiments, the variable light chain has a LCDR1, LCDR2, and a LCDR3 as set forth for PMAB12 in Table 14. In some embodiments, the variable light chain has a LCDR1, LCDR2, and a LCDR3 as set forth for PMAB13 in Table 14. In some embodiments, the variable light chain has a LCDR1, LCDR2, and a LCDR3 as set forth for PMAB14 in Table 14. In some embodiments, the variable light chain has a LCDR1, LCDR2, and a LCDR3 as set forth for PMAB15 in Table 14. In some embodiments, the variable light chain has a LCDR1, LCDR2, and a LCDR3 as set forth for PMAB16 in Table 14. In some embodiments, the variable light chain has a LCDR1, LCDR2, and a LCDR3 as set forth for PMAB1 in Table 14. In some embodiments, the variable light chain has a LCDR1, LCDR2, and a LCDR3 as set forth for PMAB19 in Table 14. In some embodiments, the variable light chain has a LCDR1, LCDR2, and a LCDR3 as set forth for PMAB20 in Table 14. In some embodiments, the variable light chain has a LCDR1, LCDR2, and a LCDR3 as set forth for PMAB21 in Table 14. In some embodiments, the variable light chain has a LCDR1, LCDR2, and a LCDR3 as set forth for PMAB22 in Table 14. In some embodiments, the variable light chain has a LCDR1, LCDR2, and a LCDR3 as set forth for PMAB23 in Table 14. In some embodiments, the variable light chain has a LCDR1, LCDR2, and a LCDR3 as set forth for PMAB24 in Table 14. In some embodiments, the variable light chain has a LCDR1, LCDR2, and a LCDR3 as set forth for PMAB25 in Table 14. In some embodiments, the variable light chain has a LCDR1, LCDR2, and a LCDR3 as set forth for PMAB26 in Table 14. In some embodiments, the variable light chain has a LCDR1, LCDR2, and a LCDR3 as set forth for PMAB27 in Table 14. In some embodiments, the variable light chain has a LCDR1, LCDR2, and a LCDR3 as set forth for PMAB28 in Table 14. In some embodiments, the variable light chain has a LCDR1, LCDR2, and a LCDR3 as set forth for PMAB29 in Table 14. In some embodiments, the variable light chain has a LCDR1, LCDR2, and a LCDR3 as set forth for PMAB30 in Table 14. In some embodiments, the variable light chain has a LCDR1, LCDR2, and a LCDR3 as set forth for PMAB31 in Table 14. In some embodiments, the variable light chain has a LCDR1, LCDR2, and a LCDR3 as set forth for PMAB32 in Table 14. In some embodiments, the variable heavy chain has a HCDR1, HCDR2, and a HCDR33 as set forth for PMAB33 in Table 14. In some embodiments, the variable light chain has a LCDR1, LCDR2, and a LCDR3 as set forth for PMAB34 in Table 14. In some embodiments, the variable light chain has a LCDR1, LCDR2, and a LCDR3 as set forth for PMAB35 in Table 14. In some embodiments, the variable light chain has a LCDR1, LCDR2, and a LCDR3 as set forth for PMAB36 in Table 14. In some embodiments, the variable light chain has a LCDR1, LCDR2, and a LCDR3 as set forth for PMAB37 in Table 14. In some embodiments, the variable light chain has a LCDR1, LCDR2, and a LCDR3 as set forth for PMAB38 in Table 14. In some embodiments, the variable light chain has a LCDR1, LCDR2, and a LCDR3 as set forth for PMAB39 in Table 14. In some embodiments, the variable light chain has a LCDR1, LCDR2, and a LCDR3 as set forth for PMAB40 in Table 14. In some embodiments, the variable light chain has a LCDR1, LCDR2, and a LCDR3 as set forth for PMAB41 in Table 14. In some embodiments, the variable light chain has a LCDR1, LCDR2, and a LCDR3 as set forth for PMAB42 in Table 14. In some embodiments, the variable light chain has a LCDR1, LCDR2, and a LCDR3 as set forth for PMAB43 in Table 14. In some embodiments, the variable light chain has a LCDR1, LCDR2, and a LCDR3 as set forth for PMAB44 in Table 14. In some embodiments, the variable light chain has a LCDR1, LCDR2, and a LCDR3 as set forth for PMAB45 in Table 14. In some embodiments, the variable light chain has a LCDR1, LCDR2, and a LCDR3 as set forth for PMAB46 in Table 14. In some embodiments, the variable light chain has a LCDR1, LCDR2, and a LCDR3 as set forth for PMAB47 in Table 14. In some embodiments, the variable light chain has a LCDR1, LCDR2, and a LCDR3 as set forth for PMAB48 in Table 14. In some embodiments, the variable light chain has a LCDR1, LCDR2, and a LCDR3 as set forth for PMAB49 in Table 14. In some embodiments, the variable light chain has a LCDR1, LCDR2, and a LCDR3 as set forth for PMAB50 in Table 14. In some embodiments, the variable light chain has a LCDR1, LCDR2, and a LCDR3 as set forth for PMAB51 in Table 14. In some embodiments, the variable light chain has a LCDR1, LCDR2, and a LCDR3 as set forth for PMAB52 in Table 14. In some embodiments, the variable light chain has a LCDR1, LCDR2, and a LCDR3 as set forth for PMAB53 in Table 14. In some embodiments, the variable light chain has a LCDR1, LCDR2, and a LCDR3 as set forth for PMAB55 in Table 14.

In some embodiments, the variable light chain has a LCDR1, LCDR2, and a LCDR3 as set forth for MIAB212 in Table 15. In some embodiments, the variable light chain has a LCDR1, LCDR2, and a LCDR3 as set forth for MIAB213 in Table 15. In some embodiments, the variable light chain has a LCDR1, LCDR2, and a LCDR3 as set forth for MIAB214 in Table 15.

In some embodiments, the CDRS are swapped for one another. For example, the HCDR1 of clone 1 can be substituted for the HCDR1 of clone 10, or vice versa. This CDR swapping can be done for any of the HCDRs of the clones provided herein (e.g., HCDR1 for HCDR1; HCDR2 for HCDR2; or HCDR3 for HCDR3) or the LCDRs (e.g., LCDR1 for LCDR1; LCDR2 for LCDR2; or LCDR3 for LCDR3). Therefore, in some embodiments, the antibody comprises a HCDR1 as set forth in any of Clones 1-66 of Table 6, Clones 1-84 of Table 7, MIAB128-198 of Table 9, PMAB1-55 of Table 14, or PMAB212-214 of Table 15; a HCDR2 as set forth in any of Clones 1-66 of Table 6, Clones 1-84 of Table 7, MIAB128-198 of Table 9, PMAB1-55 of Table 14, or PMAB212-214 of Table 15; a HCDR3 as set forth in any of Clones 1-66 of Table 6, Clones 1-84 of Table 7, MIAB128-198 of Table 9, PMAB1-55 of Table 14, or PMAB212-214 of Table 15; a LCDR1 as set forth in any of Clones 1-66 of Table 6, Clones 1-84 of Table 7, MIAB128-198 of Table 9, PMAB1-55 of Table 14, or PMAB212-214 of Table 15; a LCDR2 as set forth in any of Clones 1-66 of Table 6, Clones 1-84 of Table 7, MIAB128-198 of Table 9, PMAB1-55 of Table 14, or PMAB212-214 of Table 15; a LCDR3 as set forth in any of Clones 1-66 of Table 6, Clones 1-84 of Table 7, MIAB128-198 of Table 9, PMAB1-55 of Table 14, or PMAB212-214 of Table 15, or a variant of any of the foregoing.

In some embodiments, the MadCAM Antibody is a scFV format as shown in clones 6, 59, 63, MIAB199, MIAB200, MIAB201, MIAB202, MIAB203, MIAB204, or PMAB1-55. The linker as shown in those sequences is 20 amino acid residues in length, but could also be 5, 10, or 15 amino acid residues in length. In some embodiments, the linker the links the VH and VL (or VK) sequences of the antibody is a glycine/serine linker, which can be a sequence of GGGGSGGGGSGGGGSGGGGS (SEQ ID NO: 22) or GGGGSGGGGSGGGGS (SEQ ID NO: 30). This is simply a non-limiting example and the linker can have varying number of GGGGS (SEQ ID NO: 23) or GGGGA (SEQ ID NO: 29) repeats. In some embodiments, the linker comprises 1, 2, 3, 4, 5, 6, 7, 8, 9, or 10 of the GGGGS (SEQ ID NO: 23) or GGGGA (SEQ ID NO: 29) repeats (repeats disclosed as SEQ ID NOS 1550-1551, respectively). Thus, the linkers shown in Table 6 are non-limiting examples and can be substituted with any other linkers, such as those provided for herein.

In some embodiments, the polypeptide comprises the formula of:

(SEQ ID NOS 1552-1553, respectively)
APTSSSTKKTQLQLEHLLLDLQMILNGINNYKNPKLTRMLTFKFYMPKKA
TELKHLQCLEEELKPLEEALNLAPSKNFHLRPRDLISDINVIVLELKGSE
TTFMCEYADETATIVEFINRWITFSQSIISTLT-Linker1-
DKTHTCPPCPAPEAAGAPSVFLFPPKPKDTLMISRTPEVTCVVVDVSHED
PEVKFNWYVDGVEVHNAKTKPREEQYNSTYRVVSVLIVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPG-Linker2-Ab,

wherein Linker 1, Linker2, and Ab are as provided herein. In some embodiments, Linker 1 is GGGGSGGGGSGGGGS (SEQ ID NO: 30) or GGGGSGGGGSGGGGSGGGGS (SEQ ID NO: 22). In some embodiments, Linker 2 is GGGGS (SEQ ID NO: 23). In some embodiments, Linker 2 is GGGGSGGGGS (SEQ ID NO: 619). In some embodiments, Linker 2 is GGGGSGGGGSGGGGS (SEQ ID NO: 30). In some embodiments, Ab is the scFV as set forth in Table 6, Table 12, or Table 14. In some embodiments, the Ab comprises a sequence of SEQ ID NO: 95. In some embodiments, the Ab comprises a sequence of SEQ ID NO: 364. In some embodiments, the Ab comprises a sequence of SEQ ID NO: 386. In some embodiments, the Ab comprises a sequences of SEQ ID NOs: 41, 22, 1437, 30, 591, 22, and 592. In some embodiments, the Ab comprises a VH and a VK or VL segment. In some embodiments, the VH comprises a sequence as set forth in Table 7, Table 9, Table 10, Table 12, or Table 14. In some embodiments, the VK comprises a sequence as set forth in Table 7, Table 9, Table 10, Table 12, or Table 14. In some embodiments, the Ab comprises a VH and a VK as set forth for the clones in Table 7, Table 9, Table 10, Table 12, or Table 14. In some embodiments, the VH and VK are linked by a linker. In some embodiments, the VH and VK are linked by a peptide linker comprising a peptide of GGGGSGGGGSGGGGSGGGGS (SEQ ID NO: 22). In some embodiments, the VH and VK are linked by a peptide linker comprising a peptide of GGGGS (SEQ ID NO: 23). In some embodiments, the VH and VK are linked by a peptide linker comprising a peptide of GGGGSGGGGS (SEQ ID NO: 619). In some embodiments, the VH and VK are linked by a peptide linker comprising a peptide of GGGGSGGGGSGGGGS (SEQ ID NO: 30).

In some embodiments, the Ab comprises a VH of SEQ ID NO: 414 and a VK of SEQ ID NO: 415. In some embodiments, the Ab comprises a VH of SEQ ID NO: 591 and a VK of SEQ ID NO: 592. In some embodiments, the Ab comprises a VH of SEQ ID NO: 599 and a VK of SEQ ID NO: 600. In some embodiments, the Ab comprises a VH of SEQ ID NO: 1387 and a VK of SEQ ID NO: 1363.

In some embodiments, the peptide comprises:

(SEQ ID NO: 1554)
APTSSSTKKTQLQLEHLLLDLQMILNGINNYKNPKLTRMLTFKFYMPKKA
TELKHLQCLEEELKPLEEALNLAPSKNFHLRPRDLISDINVIVLELKGSE
TTFMCEYADETATIVEFINRWITFSQSIISTLT-(GGGGSGGGGSGGGGS
or
GGGGSGGGGSGGGGSGGGGS)-
DKTHTCPPCPAPEAAGAPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQG
NVFSCSVMHEALHNHYTQKSLSLSPG-(GGGGS
or
GGGGSGGGGS
or
GGGGSGGGGSGGGGS)-Ab,

wherein Ab is set forth as herein. In some embodiments, the Ab comprises a sequence of SEQ ID NO: 95. In some embodiments, the Ab comprises a sequence of SEQ ID NO: 364. In some embodiments, the Ab comprises a sequence of SEQ ID NO: 386. In some embodiments, the Ab comprises a VH and a VK or VL segment. In some embodiments, the VH comprises a sequence as set forth in Table 7, Table 9, Table 10, Table 12, or Table 14. In some embodiments, the VK comprises a sequence as set forth in Table 7, Table 9, Table 10, Table 12, or Table 14. In some embodiments, the Ab comprises a VH and a VK as set forth for the clones in Table 7, Table 9, Table 10, Table 12, or Table 14. In some embodiments, the VH and VK are linked by a linker. In some embodiments, the VH and VK are linked by a peptide linker comprising a peptide of GGGGSGGGGSGGGGSGGGGS (SEQ ID NO: 22). In some embodiments, the VH and VK are linked by a peptide linker comprising a peptide of GGGGS (SEQ ID NO: 23). In some embodiments, the VH and VK are linked by a peptide linker comprising a peptide of GGGGSGGGGS (SEQ ID NO: 619).

In some embodiments, the Ab comprises a VH of SEQ ID NO: 414 and a VK of SEQ ID NO: 415. In some embodiments, the Ab comprises a VH of SEQ ID NO: 591 and a VK of SEQ ID NO: 592. In some embodiments, the Ab comprises a VH of SEQ ID NO: 599 and a VK of SEQ ID NO: 600. In some embodiments, the Ab comprises a VH of SEQ ID NO: 1387 and a VK of SEQ ID NO: 1363. These examples are non-limiting the combinations of VH and VK as shown in Table 7, Table 9, Table 10, Table 12, or Table 14 are also provided.

In some embodiments, the therapeutic compound or polypeptide comprises a formula of an anti-PD-1 heavy and light chain, wherein the PD-1 heavy chain is linked to a MAdCAM antibody (scFV), such as those provided herein at the C-terminus of the PD-1 IgG heavy chain. The polypeptide can have the formula of A1-A2-Linker1-A4-Linker2-A5 and A6, wherein A1 is a PD-1 heavy chain, A6 is a PD-1 light chain; A2 is a IgG constant domain (e.g. IgG1 Constant domain), Linker 1 is as provided herein, such as, but not limited to, a glycine/serine linker, which can be a sequence of GGGGSGGGGSGGGGSGGGGS (SEQ ID NO: 22) or GGGGSGGGGSGGGGS (SEQ ID NO: 30), or GGGSEGGGSEGGGSE (SEQ ID NO: 1546) which are simply a non-limiting example and the linker can have varying number of GGGGS (SEQ ID NO: 23) or GGGGA (SEQ ID NO: 29) repeats, and in some embodiments, the linker comprises 1, 2, 3, 4, 5, 6, 7, 8, 9, or 10 of the GGGGS (SEQ ID NO: 23) or GGGGA (SEQ ID NO: 29) repeats (repeats disclosed as SEQ ID NOS 1550-1551, respectively); A4 is VH domain, such as those set forth in Table 7; Linker 2 is as provided herein, such as, but not limited to, a glycine/serine linker, which can be a sequence of GGGGSGGGGSGGGGSGGGGS (SEQ ID NO: 22) or GGGGSGGGGSGGGGS (SEQ ID NO: 30), which are simply a non-limiting example and the linker can have varying number of GGGGS (SEQ ID NO: 23) or GGGGA (SEQ ID NO: 29) repeats, and in some embodiments, the linker comprises 1, 2, 3, 4, 5, 6, 7, 8, 9, or 10 of the GGGGS (SEQ ID NO: 23) or GGGGA repeats (SEQ ID NO: 29) repeats (repeats disclosed as SEQ ID NOS 1550-1551, respectively); and A5 is VK/VL domain, such as those set forth in Table 7. In some embodiments, Linker 2 is GGGGSGGGGSGGGGS (SEQ ID NO: 30). In some embodiments, the A4-Linker2-A5 is a scFV antibody, such as those set forth in Table 6. The linkers shown in Table 6 can be substituted with the linker of GGGGSGGGGSGGGGS (SEQ ID NO: 30). In some embodiments, the A4-Linker2-A5 comprises the HCDR sets (e.g., HCDR1, HCDR2, HCDR3, LCDR1, LCDR2, and LCDR3) sets as set forth in Table 6 or Table 7. For the avoidance of doubt, a CDR set refers to the CDRs illustrated for each of the different antibody clones provided for in the tables. In some embodiments, A4 comprises a peptide of SEQ ID NO: 414 and A5 comprises a peptide of SEQ ID NO: 415. In some embodiments, A4 comprises a peptide of SEQ ID NO: 591 and A5 comprises a peptide of SEQ ID NO: 592. In some embodiments, A4 comprises a peptide of SEQ ID NO: 599 and A5 comprises a peptide of SEQ ID NO: 600. These examples are non-limiting the combinations of VH and VK as shown in Table 7, Table 12, or Table 14 are also provided.

In some embodiments, A2 comprises a sequence of

(SEQ ID NO: 44)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG.

In some embodiments, once expressed the heavy and light chains of the PD-1 antibody bind to one another to form the compound comprising the anti-PD-1 antibody linked to the anti-MAdCAM antibody. The anti-MAdCAM antibody can be any antibody that binds to MAdCAM, such as those provided for herein.

In some embodiments, the therapeutic compound comprises one or more sequences selected from the sequence in the following table

TABLE 12

| | | | Fc- | | | | | |
|---|---|---|---|---|---|---|---|---|
| | | | scFv | | Intra | | | |
| | | IgG1 Constant | Linker | scFv | scFv | ScFv | Fab | |
| Ab | Fab VH Seq | Domain Seq | Seq | VH Seq | Linker | VK Seq | VL | CK |
| PMAB | EVQLLE | ASTKGPSVFPLAP | GGGGSG | EVQLLE | GGGGSG | DIQMT | DIVMT | RTVAA |
| 1 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 591) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 2 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | SGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | GYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1347) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 3 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | SGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1439) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 4 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SYWMHW | ID NO: | SQSIS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | SGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | GYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1347) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1363) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 5 | SGGGLV | SSKSTSGGTAALG | GGGSGG | SGGGLV | | QSPSS | QSPDS | PSVFI |
| | QPGGSL | CLVKDYFPEPVTV | GGS | QPGGSL | GGGSGG | LSASV | LAVSL | FPPSD |
| | RLSCAA | SWNSGALTSGVHT | (SEQ | RLSCAA | GGSGGG | GDRVT | GERAT | EQLKS |
| | SGFTFS | FPAVLQSSGLYSL | ID NO: | SGFTFS | GS | ITCRA | INCKS | GTASV |
| | NSDMSW | SSVVTVPSSSLGT | 30) | SYWMHW | (SEQ | SQSIS | SQSVL | VCLLN |
| | VRQAPG | QTYICNVNHKPSN | | VRQAPG | ID NO: | RSLAW | YSPNN | NFYPR |
| | KGLEWV | TKVDKKVEPKSCD | | KGLEWV | 22) | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | SGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1439) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1363) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 6 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | GGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | GYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1440) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 7 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | GGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1441) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 8 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SYWMHW | ID NO: | SQSIS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | GGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | GYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1440) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1363) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 9 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SYWMHW | ID NO: | SQSIS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | GGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | QFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1441) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1363) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 10 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | GGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | GYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1442) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 11 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | GGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1395) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 12 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SYWMHW | ID NO: | SQSIS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | GGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | ETLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | GYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1442) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1363) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 13 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SYWMHW | ID NO: | SQSIS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | GGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1395) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1363) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 14 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | GYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1443) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 15 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | GGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QΥΥTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1446) | FFLYSKLTVDKSR | | 1444) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 16 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1446) | FFLYSKLTVDKSR | | 1445) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 17 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SAISGS | | GKAPK | WYQQK | WKVDN |
| | GGSTYY | AGAPSVFLFPPKP | | GGSTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | GYYMDV | | YYCQQ | AEDVA | ACEVT |
| | WGQGTD | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRI | EC |
| | 1447) | FFLYSKLTVDKSR | | 1442) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | QVQLVQ | ASTKGPSVFPLAP | GGGGSG | EVQLLE | GGGGSG | DIQMT | DIQMT | RTVAA |
| 18 | SGAEVK | SSKSTSGGTAALG | GGGSGG | SGGGLV | GGGSGG | QSPSS | QSPSS | PSVFI |
| | KPGASV | CLVKDYFPEPVTV | GGS | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | (SEQ | ITCRA | ITCQA | GTASV |
| | TYYMHW | SSVVTVPSSSLGT | 30) | SYWMHW | ID NO: | SQSIS | SRDIK | VCLLN |
| | VRQAPG | QTYICNVNHKPSN | | VRQAPG | 22) | SSLAW | NYLAW | NFYPR |
| | QGLEWM | TKVDKKVEPKSCD | | KGLEWV | | YQQKP | YQQKP | EAKVQ |
| | GIIAPS | KTHTCPPCPAPEA | | SYISGS | | GKAPK | GKAPK | WKVDN |
| | GGSTSY | AGAPSVFLFPPKP | | GGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DTSTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYDMDV | | YYCQQ | YYCQQ | ACEVT |
| | VTVSS | PPSREEMTKNQVS | | WGKGTT | | YKSYP | SYSTP | HQGLS |
| | (SEQ | LTCLVKGFYPSDI | | VTVSS | | VTFGQ | PTFGP | SPVTK |
| | ID NO: | AVEWESNGQPENN | | (SEQ | | GTKVE | GTKVD | SFNRG |
| | 1448) | YKTTPPVLDSDGS | | ID NO: | | IK | IK | EC |
| | | FFLYSKLTVDKSR | | 1445) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 1367) | 1449) | 45) |
| | | NO: 44) | | | | | | |
| PMAB | EVQLLE | ASTKGPSVFPLAP | GGGGSG | EVQLLE | GGGGSG | DIQMT | DIVMT | RTVAA |
| 19 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | SFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1353) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 20 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DYWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1391) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 21 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFAMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1450) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 22 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMSW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1451) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 23 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRI | EC |
| | 1438) | FFLYSKLTVDKSR | | 1452) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 24 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SAISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1453) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 25 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1454) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 26 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1455) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 27 | SGGGLV | SSKSTSGGTAALG | | SGGGLV | GGGSGG | QSPSS | QSPDS | PSVFI |
| | QPGGSL | CLVKDYFPEPVTV | GGGSGG | QPGGSL | GGSGGG | LSASV | LAVSL | FPPSD |
| | RLSCAA | SWNSGALTSGVHT | GGS | RLSCAA | GS | GDRVT | GERAT | EQLKS |
| | SGFTFS | FPAVLQSSGLYSL | (SEQ | SGFTFS | SEQ | ITCRA | INCKS | GTASV |
| | NSDMSW | SSVVTVPSSSLGT | ID NO: | DFWMHW | ID NO: | SQSVS | SQSVL | VCLLN |
| | VRQAPG | QTYICNVNHKPSN | 30) | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGSTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | ILTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1456) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 28 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTYY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1457) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 29 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | AASVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1458) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 30 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | AQSVKG | | ASSIQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1459) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 31 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DASKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1460) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 32 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DQSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1379) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 33 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMA | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1461) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 34 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMG | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1462) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 35 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMQ | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1463) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 36 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSIS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 591) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1363) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 37 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGç | QYYTT | SPVTK |
| | SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 591) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1364) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 38 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RYLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 591) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1365) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 39 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLNW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 591) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1366) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 40 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | SKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 591) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1464) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 41 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YYSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 591) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1465) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 42 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSTP | VYYCQ | H?GLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 591) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1466) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 43 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | RTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 591) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1467) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 44 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPT | | EDFAT | ISSLQ | KHKVY |
| | SGWLDY | AKGQPREPQVYTL | | YYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1390) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 45 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | DYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1468) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 46 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | GYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1469) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 47 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | NYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1470) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 48 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSI | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | SYYMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1471) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 49 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1472) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 50 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYEMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1473) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 51 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYKMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1474) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 52 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | NSDMSW | SSVVTVPSSSLGT | 30) | DFWMHW | ID NO: | SQSVS | SQSVL | VCLLN |
| | VRQAPG | QTYICNVNHKPSN | | VRQAPG | 22) | RSLAW | YSPNN | NFYPR |
| | KGLEWV | TKVDKKVEPKSCD | | KGLEWI | | YQQKP | KNYLA | EAKVQ |
| | SGITIS | KTHTCPPCPAPEA | | SYISGD | | GKAPK | WYQQK | WKVDN |
| | GGSTYY | AGAPSVFLFPPKP | | SGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYSMDV | | YYCQQ | AEDVA | ACEVT |
| | WGQGTL | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1438) | FFLYSKLTVDKSR | | 1475) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 592) | NO: | 45) |
| | | NO: 44) | | | | | 1359) | |
| PMAB | EVQLLE | ASTKGPSVFPLAP | GGGGSG | EVQLLE | GGGGSG | DIQMT | DIVMT | RTVAA |
| 53 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDIDV | | YYCQQ | AEDVA | ACEVT |
| | WGQGTT | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1476) | FFLYSKLTVDKSR | | 1477) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLVQ | ASTKGPSVFPLAP | GGGGSG | EVQLLE | GGGGSG | DIQMT | DIQMT | RTVAA |
| 54 | SGAEVK | SSKSTSGGTAALG | GGGSGG | SGGGLV | GGGSGG | QSPSS | QSPSS | PSVFI |
| | KPGASV | CLVKDYFPEPVTV | GGS | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | (SEQ | ITCRA | ITCRA | GTASV |
| | SYYMHW | SSVVTVPSSSLGT | 30) | SYWMHW | ID NO: | SQSIS | SQSIS | VCLLN |
| | VRQAPG | QTYICNVNHKPSN | | VRQAPG | 22) | SSLAW | SYLAW | NFYPR |
| | QGLEWM | TKVDKKVEPKSCD | | KGLEWV | | YQQKP | YQQKP | EAKVQ |
| | GIINPS | KTHTCPPCPAPEA | | SYISGS | | GKAPK | GKAPK | WKVDN |
| | GGSTSY | AGAPSVFLFPPKP | | GGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DTSTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYDIDV | | YYCQQ | YYCQQ | ACEVT |
| | VTVSS | PPSREEMTKNQVS | | WGKGTT | | YKSYP | SYSTP | H?GLS |
| | (SEQ | LTCLVKGFYPSDI | | VTVSS | | VTFGQ | PTFGP | SPVTK |
| | ID NO: | AVEWESNGQPENN | | (SEQ | | GTKVE | GTKVD | SFNRG |
| | 1478) | YKTTPPVLDSDGS | | ID NO: | | IK | IK | EC |
| | | FFLYSKLTVDKSR | | 1477) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 1367) | 1479) | 45) |
| | | NO: 44) | | | | | | |
| PMAB | EVQLVQ | ASTKGPSVFPLAP | GGGGSG | EVQLLE | GGGGSG | DIQMT | DIQMT | RTVAA |
| 55 | SGAEVK | SSKSTSGGTAALG | GGGSGG | SGGGLV | GGGSGG | QSPSS | QSPSS | PSVFI |
| | KPGASV | CLVKDYFPEPVTV | GGS | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | SEQ | ITCRA | ITCRA | GTASV |
| | SYYMHW | SSVVTVPSSSLGT | 30) | SYWMHW | ID NO: | SQSIS | SQSIS | VCLLN |
| | VRQAPG | QTYICNVNHKPSN | | VRQAPG | 22) | SSLAW | SYLAW | NFYPR |
| | QGLEWM | TKVDKKVEPKSCD | | KGLEWV | | YQQKP | YQQKP | EAKVQ |
| | GIINPS | KTHTCPPCPAPEA | | SYISGS | | GKAPK | GKAPK | WKVDN |
| | GGSTSY | AGAPSVFLFPPKP | | GGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DTSTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYDLDV | | YYCQQ | YYCQQ | ACEVT |
| | VTVSS | PPSREEMTKNQVS | | WGKGTT | | YKSYP | SYSTP | HQGLS |
| | (SEQ | LTCLVKGFYPSDI | | VTVSS | | VTFGQ | PTFGP | SPVTK |
| | ID NO: | AVEWESNGQPENN | | (SEQ | | GTKVE | GTKVD | SFNRG |
| | 1478) | YKTTPPVLDSDGS | | ID NO: | | IK | IK | EC |
| | | FFLYSKLTVDKSR | | 1480) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 1367) | 1479) | 45) |
| | | NO: 44) | | | | | | |
| PMAB | EVQLLE | ASTKGPSVFPLAP | GGGGSG | EVQLLE | GGGGSG | DIQMT | DIVMT | RTVAA |
| 56 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDLDV | | YYCQQ | AEDVA | ACEVT |
| | WGQGTT | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1476) | FFLYSKLTVDKSR | | 1480) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | QVQLVQ | ASTKGPSVFPLAP | GGGGSG | EVQLLE | GGGGSG | DIQMT | DIQMT | RTVAA |
| 57 | SGAEVK | SSKSTSGGTAALG | GGGSGG | SGGGLV | GGGSGG | QSPSS | QSPSS | PSVFI |
| | KPGASV | CLVKDYFPEPVTV | GGS | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | (SEQ | ITCRA | ITCQA | GTASV |
| | TYYMHW | SSVVTVPSSSLGT | 30) | DFWMHW | ID NO: | SQSVS | SRDIK | VCLLN |
| | VRQAPG | QTYICNVNHKPSN | | VRQAPG | 22) | RSLAW | NYLAW | NFYPR |
| | QGLEWM | TKVDKKVEPKSCD | | KGLEWI | | YQQKP | YQQKP | EAKVQ |
| | GIIAPS | KTHTCPPCPAPEA | | SYISGD | | GKAPK | GKAPK | WKVDN |
| | GGSTSY | AGAPSVFLFPPKP | | SGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DTSTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYYMDV | | YYCQQ | YYCQQ | ACEVT |
| | VTVSS | PPSREEMTKNQVS | | WGKGTT | | YKSYP | SYSTP | HQGLS |
| | (SEQ | LTCLVKGFYPSDI | | VTVSS | | VTFGQ | PTFGP | SPVTK |
| | ID NO: | AVEWESNGQPENN | | (SEQ | | GTKVE | GTKVD | SFNRG |
| | 1448) | YKTTPPVLDSDGS | | ID NO: | | IK | IK | EC |
| | | FFLYSKLTVDKSR | | 591) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 592) | 1449) | 45) |
| | | NO: 44) | | | | | | |
| PMAB | EVQLLE | ASTKGPSVFPLAP | GGGGSG | EVQLLE | GGGGSG | DIQMT | DIVMT | RTVAA |
| 60 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KCLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDMDV | | YYCQQ | AEDVA | ACEVT |
| | WGQGTT | PPSREEMTKNQVS | | WGKGTT | | YKSYP | VYYCQ | HQGLS |
| | VTVSS | LTCLVKGFYPSDI | | VTVSS | | VTFGC | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1476) | FFLYSKLTVDKSR | | 1542) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1543) | NO: | 45) |
| | | NO: 44) | | | | | 1359) | |
| PMAB | EVQLVQ | ASTKGPSVFPLAP | GGGGSG | EVQLLE | GGGGSG | DIQMT | DIQMT | RTVAA |
| 61 | SGAEVK | SSKSTSGGTAALG | GGGSGG | SGGGLV | GGGSGG | QSPSS | QSPSS | PSVFI |
| | KPGASV | CLVKDYFPEPVTV | GGS | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | (SEQ | ITCRA | ITCRA | GTASV |
| | SYYMHW | SSVVTVPSSSLGT | 30) | SYWMHW | ID NO: | SQSIS | SQSIS | VCLLN |
| | VRQAPG | QTYICNVNHKPSN | | VRQAPG | 22) | SSLAW | SYLAW | NFYPR |
| | QGLEWM | TKVDKKVEPKSCD | | KCLEWV | | YQQKP | YQQKP | EAKVQ |
| | GIINPS | KTHTCPPCPAPEA | | SYISGS | | GKAPK | GKAPK | WKVDN |
| | GGSTSY | AGAPSVFLFPPKP | | GGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DTSTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYDMDV | | YYCQQ | YYCQQ | ACEVT |
| | VTVSS | PPSREEMTKNQVS | | WGKGTT | | YKSYP | SYSTP | HQGLS |
| | (SEQ | LTCLVKGFYPSDI | | VTVSS | | VTFGC | PTFGP | SPVTK |
| | ID NO: | AVEWESNGQPENN | | (SEQ | | GTKVE | GTKVD | SFNRG |
| | 1478) | YKTTPPVLDSDGS | | ID NO: | | IK | IK | EC |
| | | FFLYSKLTVDKSR | | 1542) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 1543) | 1479) | 45) |
| | | NO: 44) | | | | | | |
| PMAB | EVQLLE | ASTKGPSVFPLAP | GGGGSG | EVQLLE | GGGGSG | DIQMT | DIVMT | RTVAA |
| 62 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KCLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDIDV | | YYCQQ | AEDVA | ACEVT |
| | WGOGTT | PPSREEMTKNQVS | | WGKGTT | | YKSYP | VYYCQ | HQGLS |
| | VTVSS | LTCLVKGFYPSDI | | VTVSS | | VTFGC | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1476) | FFLYSKLTVDKSR | | 1544) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1543) | NO: | 45) |
| | | NO: 44) | | | | | 1359) | |
| PMAB | EVQLVQ | ASTKGPSVFPLAP | GGGGSG | EVQLLE | GGGGSG | DIQMT | DIQMT | RTVAA |
| 63 | SGAEVK | SSKSTSGGTAALG | GGGSGG | SGGGLV | GGGSGG | QSPSS | QSPSS | PSVFI |
| | KPGASV | CLVKDYFPEPVTV | GGS | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | (SEQ | ITCRA | ITCRA | GTASV |
| | SYYMHW | SSVVTVPSSSLGT | 30) | SYWMHW | ID NO: | SQSIS | SQSIS | VCLLN |
| | VRQAPG | QTYICNVNHKPSN | | VRQAPG | 22) | SSLAW | SYLAW | NFYPR |
| | QGLEWM | TKVDKKVEPKSCD | | KCLEWV | | YQQKP | YQQKP | EAKVQ |
| | GIINPS | KTHTCPPCPAPEA | | SYISGS | | GKAPK | GKAPK | WKVDN |
| | GGSTSY | AGAPSVFLFPPKP | | GGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DTSTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYDIDV | | YYCQQ | YYCQQ | ACEVT |
| | VTVSS | PPSREEMTKNQVS | | WGKGTT | | YKSYP | SYSTP | HQGLS |
| | (SEQ | LTCLVKGFYPSDI | | VTVSS | | VTFGC | PTFGP | SPVTK |
| | ID NO: | AVEWESNGQPENN | | (SEQ | | GTKVE | GTKVD | SFNRG |
| | 1478) | YKTTPPVLDSDGS | | ID NO: | | IK | IK | EC |
| | | FFLYSKLTVDKSR | | 1544) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 1543) | 1479) | 45) |
| | | NO: 44) | | | | | | |
| PMAB | EVQLVQ | ASTKGPSVFPLAP | GGGGSG | EVQLLE | GGGGSG | DIQMT | DIQMT | RTVAA |
| 64 | SGAEVK | SSKSTSGGTAALG | GGGSGG | SGGGLV | GGGSGG | QSPSS | QSPSS | PSVFI |
| | KPGASV | CLVKDYFPEPVTV | GGS | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | (SEQ | ITCRA | ITCRA | GTASV |
| | SYYMHW | SSVVTVPSSSLGT | 30) | SYWMHW | ID NO: | SQSIS | SQSIS | VCLLN |
| | VRQAPG | QTYICNVNHKPSN | | VRQAPG | 22) | SSLAW | SYLAW | NFYPR |
| | QGLEWM | TKVDKKVEPKSCD | | KCLEWV | | YQQKP | YQQKP | EAKVQ |
| | GIINPS | KTHTCPPCPAPEA | | SYISGS | | GKAPK | GKAPK | WKVDN |
| | GGSTSY | AGAPSVFLFPPKP | | GGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DTSTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYDLDV | | YYCQQ | YYCQQ | ACEVT |
| | VTVSS | PPSREEMTKNQVS | | WGKGTT | | YKSYP | SYSTP | HQGLS |
| | (SEQ | LTCLVKGFYPSDI | | VTVSS | | VTFGC | PTFGP | SPVTK |
| | ID NO: | AVEWESNGQPENN | | (SEQ | | GTKVE | GTKVD | SFNRG |
| | 1478) | YKTTPPVLDSDGS | | ID NO: | | IK | IK | EC |
| | | FFLYSKLTVDKSR | | 1545) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 1543) | 1479) | 45) |
| | | NO: 44) | | | | | | |
| PMAB | EVQLLE | ASTKGPSVFPLAP | GGGGSG | EVQLLE | GGGGSG | DIQMT | DIVMT | RTVAA |
| 65 | SGGGLV | SSKSTSGGTAALG | GGGSGG | SGGGLV | GGGSGG | QSPSS | QSPDS | PSVFI |
| | QPGGSL | CLVKDYFPEPVTV | GGS | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 30) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KCLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDLDV | | YYCQQ | AEDVA | ACEVT |
| | WGQGTT | PPSREEMTKNQVS | | WGKGTT | | YKSYP | VYYCQ | HQGLS |
| | VTVSS | LTCLVKGFYPSDI | | VTVSS | | VTFGC | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1476) | FFLYSKLTVDKSR | | 1545) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1543) | NO: | 45) |
| | | NO: 44) | | | | | 1359) | |
| PMAB | EVQLLE | ASTKGPSVFPLAP | GGGSEG | EVQLLE | GGGGSG | DIQMT | DIVMT | RTVAA |
| 66 | SGGGLV | SSKSTSGGTAALG | GGSEGG | SGGGLV | GGGSGG | QSPSS | QSPDS | PSVFI |
| | QPGGSL | CLVKDYFPEPVTV | GSE | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 1546) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDMDV | | YYCQQ | AEDVA | ACEVT |
| | WGOGTT | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1476) | FFLYSKLTVDKSR | | 1445) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLVQ | ASTKGPSVFPLAP | GGGSEG | EVQLLE | GGGGSG | DIQMT | DIQMT | RTVAA |
| 67 | SGAEVK | SSKSTSGGTAALG | GGSEGG | SGGGLV | GGGSGG | QSPSS | QSPSS | PSVFI |
| | KPGASV | CLVKDYFPEPVTV | GSE | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | (SEQ | ITCRA | ITCRA | GTASV |
| | SYYMHW | SSVVTVPSSSLGT | 1546) | SYWMHW | ID NO: | SQSIS | SQSIS | VCLLN |
| | VRQAPG | QTYICNVNHKPSN | | VRQAPG | 22) | SSLAW | SYLAW | NFYPR |
| | QGLEWM | TKVDKKVEPKSCD | | KGLEWV | | YQQKP | YQQKP | EAKVQ |
| | GIINPS | KTHTCPPCPAPEA | | SYISGS | | GKAPK | GKAPK | WKVDN |
| | GGSTSY | AGAPSVFLFPPKP | | GGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DTSTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYDMDV | | YYCQQ | YYCQQ | ACEVT |
| | VTVSS | PPSREEMTKNQVS | | WGKGTT | | YKSYP | SYSTP | HQGLS |
| | (SEQ | LTCLVKGFYPSDI | | VTVSS | | VTFGQ | PTFGP | SPVTK |
| | ID NO: | AVEWESNGQPENN | | (SEQ | | GTKVE | GTKVD | SFNRG |
| | 1478) | YKTTPPVLDSDGS | | ID NO: | | IK | IK | EC |
| | | FFLYSKLTVDKSR | | 1445) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 1367) | 1479) | 45) |
| | | NO: 44) | | | | | | |
| PMAB | EVQLLE | ASTKGPSVFPLAP | GGGSEG | EVQLLE | GGGGSG | DIQMT | DIVMT | RTVAA |
| 68 | SGGGLV | SSKSTSGGTAALG | GGSEGG | SGGGLV | GGGSGG | QSPSS | QSPDS | PSVFI |
| | QPGGSL | CLVKDYFPEPVTV | GSE | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 1546) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDIDV | | YYCQQ | AEDVA | ACEVT |
| | WGQGTT | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1476) | FFLYSKLTVDKSR | | 1477) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLVQ | ASTKGPSVFPLAP | GGGSEG | EVQLLE | GGGGSG | DIQMT | DIQMT | RTVAA |
| 69 | SGAEVK | SSKSTSGGTAALG | GGSEGG | SGGGLV | GGGSGG | QSPSS | QSPSS | PSVFI |
| | KPGASV | CLVKDYFPEPVTV | GSE | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | (SEQ | ITCRA | ITCRA | GTASV |
| | SYYMHW | SSVVTVPSSSLGT | 1546) | SYWMHW | ID NO: | SQSIS | SQSIS | VCLLN |
| | VRQAPG | QTYICNVNHKPSN | | VRQAPG | 22) | SSLAW | SYLAW | NFYPR |
| | QGLEWM | TKVDKKVEPKSCD | | KGLEWV | | YQQKP | YQQKP | EAKVQ |
| | GIINPS | KTHTCPPCPAPEA | | SYISGS | | GKAPK | GKAPK | WKVDN |
| | GGSTSY | AGAPSVFLFPPKP | | GGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DTSTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYDIDV | | YYCQQ | YYCQQ | ACEVT |
| | VTVSS | PPSREEMTKNQVS | | WGKGTT | | YKSYP | SYSTP | HQGLS |
| | SEQ | LTCLVKGFYPSDI | | VTVSS | | VTFGQ | PTFGP | SPVTK |
| | ID NO: | AVEWESNGQPENN | | (SEQ | | GTKVE | GTKVD | SFNRG |
| | 1478) | YKTTPPVLDSDGS | | ID NO: | | IK | IK | EC |
| | | FFLYSKLTVDKSR | | 1477) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 1367) | 1479) | 45) |
| | | NO: 44) | | | | | | |
| PMAB | EVQLVQ | ASTKGPSVFPLAP | GGGSEG | EVQLLE | GGGGSG | DIQMT | DIQMT | RTVAA |
| 70 | SGAEVK | SSKSTSGGTAALG | GGSEGG | SGGGLV | GGGSGG | QSPSS | QSPSS | PSVFI |
| | KPGASV | CLVKDYFPEPVTV | GSE | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | (SEQ | ITCRA | ITCRA | GTASV |
| | SYYMHW | SSVVTVPSSSLGT | 1546) | SYWMHW | ID NO: | SQSIS | SQSIS | VCLLN |
| | VRQAPG | QTYICNVNHKPSN | | VRQAPG | 22) | SSLAW | SYLAW | NFYPR |
| | QGLEWM | TKVDKKVEPKSCD | | KGLEWV | | YQQKP | YQQKP | EAKVQ |
| | GIINPS | KTHTCPPCPAPEA | | SYISGS | | GKAPK | GKAPK | WKVDN |
| | GGSTSY | AGAPSVFLFPPKP | | GGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DTSTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYDLDV | | YYCQQ | YYCQQ | ACEVT |
| | VTVSS | PPSREEMTKNQVS | | WGKGTT | | YKSYP | SYSTP | HQGLS |
| | (SEQ | LTCLVKGFYPSDI | | VTVSS | | VTFGQ | PTFGP | SPVTK |
| | ID NO: | AVEWESNGQPENN | | (SEQ | | GTKVE | GTKVD | SFNRG |
| | 1478) | YKTTPPVLDSDGS | | ID NO: | | IK | IK | EC |
| | | FFLYSKLTVDKSR | | 1480) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 1367) | 1479) | 45) |
| | | NO: 44) | | | | | | |
| PMAB | EVQLLE | ASTKGPSVFPLAP | GGGSEG | EVQLLE | GGGGSG | DIQMT | DIVMT | RTVAA |
| 71 | SGGGLV | SSKSTSGGTAALG | GGSEGG | SGGGLV | GGGSGG | QSPSS | QSPDS | PSVFI |
| | QPGGSL | CLVKDYFPEPVTV | GSE | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 1546) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KGLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDLDV | | YYCQQ | AEDVA | ACEVT |
| | WGQGTT | PPSREEMTKNQVS | | WGKGTT | | YKSYP | VYYCQ | HQGLS |
| | VTVSS | LTCLVKGFYPSDI | | VTVSS | | VTFGQ | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1476) | FFLYSKLTVDKSR | | 1480) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1367) | NO: | 45) |
| | | NO: 44) | | | | | 1359) | |
| PMAB | EVQLLE | ASTKGPSVFPLAP | GGGSEG | EVQLLE | GGGGSG | DIQMT | DIVMT | RTVAA |
| 72 | SGGGLV | SSKSTSGGTAALG | GGSEGG | SGGGLV | GGGSGG | QSPSS | QSPDS | PSVFI |
| | QPGGSL | CLVKDYFPEPVTV | GSE | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 1546) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KCLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDMDV | | YYCQQ | AEDVA | ACEVT |
| | WGQGTT | PPSREEMTKNQVS | | WGKGTT | | YKSYP | VYYCQ | HQGLS |
| | VTVSS | LTCLVKGFYPSDI | | VTVSS | | VTFGC | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1476) | FFLYSKLTVDKSR | | 1542) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1543) | NO: | 45) |
| | | NO: 44) | | | | | 1359) | |
| PMAB | EVQLVQ | ASTKGPSVFPLAP | GGGSEG | EVQLLE | GGGGSG | DIQMT | DIQMT | RTVAA |
| 73 | SGAEVK | SSKSTSGGTAALG | GGSEGG | SGGGLV | GGGSGG | QSPSS | QSPSS | PSVFI |
| | KPGASV | CLVKDYFPEPVTV | GSE | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | (SEQ | ITCRA | ITCRA | GTASV |
| | SYYMHW | SSVVTVPSSSLGT | 1546) | SYWMHW | ID NO: | SQSIS | SQSIS | VCLLN |
| | VRQAPG | QTYICNVNHKPSN | | VRQAPG | 22) | SSLAW | SYLAW | NFYPR |
| | QGLEWM | TKVDKKVEPKSCD | | KCLEWV | | YQQKP | YQQKP | EAKVQ |
| | GIINPS | KTHTCPPCPAPEA | | SYISGS | | GKAPK | GKAPK | WKVDN |
| | GGSTSY | AGAPSVFLFPPKP | | GGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DTSTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYDMDV | | YYCQQ | YYCQQ | ACEVT |
| | VTVSS | PPSREEMTKNQVS | | WGKGTT | | YKSYP | SYSTP | HQGLS |
| | (SEQ | LTCLVKGFYPSDI | | VTVSS | | VTFGC | PTFGP | SPVTK |
| | ID NO: | AVEWESNGQPENN | | (SEQ | | GTKVE | GTKVD | SFNRG |
| | 1478) | YKTTPPVLDSDGS | | ID NO: | | IK | IK | EC |
| | | FFLYSKLTVDKSR | | 1542) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 1543) | 1479) | 45) |
| | | NO: 44) | | | | | | |
| PMAB | EVQLLE | ASTKGPSVFPLAP | GGGSEG | EVQLLE | GGGGSG | DIQMT | DIVMT | RTVAA |
| 74 | SGGGLV | SSKSTSGGTAALG | GGSEGG | SGGGLV | GGGSGG | QSPSS | QSPDS | PSVFI |
| | QPGGSL | CLVKDYFPEPVTV | GSE | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 1546) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KCLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDIDV | | YYCQQ | AEDVA | ACEVT |
| | WGQGTT | PPSREEMTKNQVS | | WGKGTT | | YKSYP | VYYCQ | HQGLS |
| | VTVSS | LTCLVKGFYPSDI | | VTVSS | | VTFGC | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1476) | FFLYSKLTVDKSR | | 1544) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1543) | NO: | 45) |
| | | NO: 44) | | | | | 1359) | |
| PMAB | EVQLVQ | ASTKGPSVFPLAP | GGGSEG | EVQLLE | GGGGSG | DIQMT | DIQMT | RTVAA |
| 75 | SGAEVK | SSKSTSGGTAALG | GGSEGG | SGGGLV | GGGSGG | QSPSS | QSPSS | PSVFI |
| | KPGASV | CLVKDYFPEPVTV | GSE | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | (SEQ | ITCRA | ITCRA | GTASV |
| | SYYMHW | SSVVTVPSSSLGT | 1546) | SYWMHW | ID NO: | SQSIS | SQSIS | VCLLN |
| | VRQAPG | QTYICNVNHKPSN | | VRQAPG | 22) | SSLAW | SYLAW | NFYPR |
| | QGLEWM | TKVDKKVEPKSCD | | KCLEWV | | YQQKP | YQQKP | EAKVQ |
| | GIINPS | KTHTCPPCPAPEA | | SYISGS | | GKAPK | GKAPK | WKVDN |
| | GGSTSY | AGAPSVFLFPPKP | | GGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DISTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYDIDV | | YYCQQ | YYCQQ | ACEVT |
| | VTVSS | PPSREEMTKNQVS | | WGKGTT | | YKSYP | SYSTP | HQGLS |
| | (SEQ | LTCLVKGFYPSDI | | VTVSS | | VTFGC | PTFGP | SPVTK |
| | ID NO: | AVEWESNGQPENN | | (SEQ | | GTKVE | GTKVD | SFNRG |
| | 1478) | YKTTPPVLDSDGS | | ID NO: | | IK | IK | EC |
| | | FFLYSKLTVDKSR | | 1544) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 1543) | 1479) | 45) |
| | | NO: 44) | | | | | | |
| PMAB | EVQLVQ | ASTKGPSVFPLAP | GGGSEG | EVQLLE | GGGGSG | DIQMT | DIQMT | RTVAA |
| 76 | SGAEVK | SSKSTSGGTAALG | GGSEGG | SGGGLV | GGGSGG | QSPSS | QSPSS | PSVFI |
| | KPGASV | CLVKDYFPEPVTV | GSE | QPGGSL | GGSGGG | LSASV | LSASV | FPPSD |
| | KVSCKA | SWNSGALTSGVHT | (SEQ | RLSCAA | GS | GDRVT | GDRVT | EQLKS |
| | SGYSFT | FPAVLQSSGLYSL | ID NO: | SGFTFS | (SEQ | ITCRA | ITCRA | GTASV |
| | SYYMHW | SSVVTVPSSSLGT | 1546) | SYWMHW | ID NO: | SQSIS | SQSIS | VCLLN |
| | VRQAPG | QTYICNVNHKPSN | | VRQAPG | 22) | SSLAW | SYLAW | NFYPR |
| | QGLEWM | TKVDKKVEPKSCD | | KCLEWV | | YQQKP | YQQKP | EAKVQ |
| | GIINPS | KTHTCPPCPAPEA | | SYISGS | | GKAPK | GKAPK | WKVDN |
| | GGSTSY | AGAPSVFLFPPKP | | GGYTNY | | LLIYA | LLIYA | ALQSG |
| | AQKFQG | KDTLMISRTPEVT | | ADSVKG | | ASSLQ | ASSLQ | NSQES |
| | RVTMTR | CVVVDVSHEDPEV | | RFTISR | | SGVPS | SGVPS | VTEQD |
| | DTSTST | KFNWYVDGVEVHN | | DNSKNT | | RFSGS | RFSGS | SKDST |
| | VYMELS | AKTKPREEQYNST | | LYLQMN | | GSGTD | GSGTD | YSLSS |
| | SLRSED | YRVVSVLTVLHQD | | SLRAED | | FTLTI | FTLTI | TLTLS |
| | TAVYYC | WLNGKEYKCKVSN | | TAVYYC | | SSLQP | SSLQP | KADYE |
| | ASGWVY | KALPAPIEKTISK | | ARDRPY | | EDFAT | EDFAT | KHKVY |
| | WGQGTL | AKGQPREPQVYTL | | YYDLDV | | YYCQQ | YYCQQ | ACEVT |
| | VTVSS | PPSREEMTKNQVS | | WGKGTT | | YKSYP | SYSTP | HQGLS |
| | (SEQ | LTCLVKGFYPSDI | | VTVSS | | VTFGC | PTFGP | SPVTK |
| | ID NO: | AVEWESNGQPENN | | (SEQ | | GTKVE | GTKVD | SFNRG |
| | 1478) | YKTTPPVLDSDGS | | ID NO: | | IK | IK | EC |
| | | FFLYSKLTVDKSR | | 1545) | | (SEQ | (SEQ | (SEQ |
| | | WQQGNVFSCSVMH | | | | ID | ID | ID |
| | | EALHNHYTQKSLS | | | | NO: | NO: | NO: |
| | | LSPG (SEQ ID | | | | 1543) | 1479) | 45) |
| | | NO: 44) | | | | | | |
| PMAB | EVQLLE | ASTKGPSVFPLAP | GGGSEG | EVQLLE | GGGGSG | DIQMT | DIVMT | RTVAA |
| 77 | SGGGLV | SSKSTSGGTAALG | GGSEGG | SGGGLV | GGGSGG | QSPSS | QSPDS | PSVFI |
| | QPGGSL | CLVKDYFPEPVTV | GSE | QPGGSL | GGSGGG | LSASV | LAVSL | FPPSD |
| | RLSCAA | SWNSGALTSGVHT | (SEQ | RLSCAA | GS | GDRVT | GERAT | EQLKS |
| | SGFTFS | FPAVLQSSGLYSL | ID NO: | SGFTFS | (SEQ | ITCRA | INCKS | GTASV |
| | SYDMSW | SSVVTVPSSSLGT | 1546) | SYWMHW | ID NO: | SQSIS | SQSVL | VCLLN |
| | VRQAPG | QTYICNVNHKPSN | | VRQAPG | 22) | SSLAW | YSPNN | NFYPR |
| | KGLEWV | TKVDKKVEPKSCD | | KCLEWV | | YQQKP | KNYLA | EAKVQ |
| | SGITIS | KTHTCPPCPAPEA | | SYISGS | | GKAPK | WYQQK | WKVDN |
| | GGSTYY | AGAPSVFLFPPKP | | GGYTNY | | LLIYA | PGQPP | ALQSG |
| | ADSVKG | KDTLMISRTPEVT | | ADSVKG | | ASSLQ | KLLIY | NSQES |
| | RFTISR | CVVVDVSHEDPEV | | RFTISR | | SGVPS | WASTR | VTEQD |
| | DNSKNT | KFNWYVDGVEVHN | | DNSKNT | | RFSGS | ESGVP | SKDST |
| | LYLQMN | AKTKPREEQYNST | | LYLQMN | | GSGTD | DRFSG | YSLSS |
| | SLRAED | YRVVSVLTVLHQD | | SLRAED | | FTLTI | SGSGT | TLTLS |
| | TAVYYC | WLNGKEYKCKVSN | | TAVYYC | | SSLQP | DFTLT | KADYE |
| | ARGRGG | KALPAPIEKTISK | | ARDRPY | | EDFAT | ISSLQ | KHKVY |
| | SGWLDY | AKGQPREPQVYTL | | YYDLDV | | YYCQQ | AEDVA | ACEVT |
| | WGQGTT | PPSREEMTKNQVS | | WGKGTT | | YKSYP | VYYCQ | HQGLS |
| | VTVSS | LTCLVKGFYPSDI | | VTVSS | | VTFGC | QYYTT | SPVTK |
| | (SEQ | AVEWESNGQPENN | | (SEQ | | GTKVE | PPTFG | SFNRG |
| | ID NO: | YKTTPPVLDSDGS | | ID NO: | | IK | QGTRL | EC |
| | 1476) | FFLYSKLTVDKSR | | 1545) | | (SEQ | EIK | (SEQ |
| | | WQQGNVFSCSVMH | | | | ID | (SEQ | ID |
| | | EALHNHYTQKSLS | | | | NO: | ID | NO: |
| | | LSPG (SEQ ID | | | | 1543) | NO: | 45) |
| | | NO: 44) | | | | | 1359) | |

In some embodiments, the therapeutic compound comprises one or more sequences, or a combination thereof, selected from the Table 12.

In some embodiments, the PD-1-MAdCAM antibody comprises an anti-PD-1 Fab as provided for in the following table:

TABLE 13

| Clone | | | | | | | | |
|---|---|---|---|---|---|---|---|---|
| (Fab) | Fab VH Seq | Fab VL Seq | HCDR1 | HCDR2 | HCDR3 | LCDR1 | LCDR2 | LCDR3 |
| PMAB1 | EVQLLESGGG | DIVMTQSPDS | FTFSN | VSGIT | ARGRG | KSSQS | WASTR | QQYYT |
| | LVQPGGSLRL | LAVSLGERAT | SDMS | ISGGS | GSGWL | VLYSP | ES | TPPT |
| | SCAASGFTFS | INCKSSQSVL | (SEQ | TYYA | DY | NNKNY | (SEQ | (SEQ |
| | NSDMSWVRQA | YSPNNKNYLA | ID | (SEQ | (SEQ | LA | ID | ID |
| | PGKGLEWVSG | WYQQKPGQPP | NO: | ID | ID | (SEQ | NO: | NO: |
| | ITISGGSTYY | KLLIYWASTR | 1481) | NO: | NO: | ID | 1485) | 1486) |
| | ADSVKGRFTI | ESGVPDRFSG | | 1482) | 1483) | NO: | | |
| | SRDNSKNTLY | SGSGTDFTLT | | | | 1484) | | |
| | LQMNSLRAED | ISSLQAEDVA | | | | | | |
| | TAVYYCARGR | VYYCQQYYTT | | | | | | |
| | GGSGWLDYWG | PPTFGQGTRL | | | | | | |
| | QGTLVTVSS | EIK (SEQ | | | | | | |
| | (SEQ ID | ID NO: | | | | | | |
| | NO: 1438) | 1359) | | | | | | |
| PMAB15 | EVQLLESGGG | DIVMTQSPDS | FTFSS | VSGIT | ARGRG | KSSQS | WASTR | QQYYT |
| | LVQPGGSLRL | LAVSLGERAT | YDMS | ISGGS | GSGWL | VLYSP | ES | TPPT |
| | SCAASGFTFS | INCKSSQSVL | (SEQ | TYYA | DY | NNKNY | (SEQ | (SEQ |
| | SYDMSWVRQA | YSPNNKNYLA | ID | (SEQ | (SEQ | LA | ID | ID |
| | PGKGLEWVSG | WYQQKPGQPP | NO: | ID | ID | (SEQ | NO: | NO: |
| | ITISGGSTYY | KLLIYWASTR | 1487) | NO: | NO: | ID | 1485) | 1486) |
| | ADSVKGRFTI | ESGVPDRFSG | | 1482) | 1483) | NO: | | |
| | SRDNSKNTLY | SGSGTDFTLT | | | | 1484) | | |
| | LQMNSLRAED | ISSLQAEDVA | | | | | | |
| | TAVYYCARGR | VYYCQQYYTT | | | | | | |
| | GGSGWLDYWG | PPTFGQGTRL | | | | | | |
| | QGTLVTVSS | EIK (SEQ | | | | | | |
| | (SEQ ID | ID NO: | | | | | | |
| | NO: 1446) | 1359) | | | | | | |
| PMAB17 | EVQLLESGGG | DIVMTQSPDS | FTFSS | VSGIT | ARGRG | KSSQS | WASTR | QQYYT |
| | LVQPGGSLRL | LAVSLGERAT | YDMS | ISGGS | GSGWL | VLYSP | ES | TPPT |
| | SCAASGFTES | INCKSSQSVL | (SEQ | TYYA | DY | NNKNY | (SEQ | (SEQ |
| | SYDMSWVRQA | YSPNNKNYLA | ID | (SEQ | (SEQ | LA | ID | ID |
| | PGKGLEWVSG | WYQQKPGQPP | NO: | ID | ID | (SEQ | NO: | NO: |
| | ITISGGSTYY | KLLIYWASTR | 1487) | NO: | NO: | ID | 1485) | 1486) |
| | ADSVKGRFTI | ESGVPDRESG | | 1482) | 1483) | NO: | | |
| | SRDNSKNTLY | SGSGTDFTLT | | | | 1484) | | |
| | LQMNSLRAED | ISSLQAEDVA | | | | | | |
| | TAVYYCARGR | VYYCQQYYTT | | | | | | |
| | GGSGWLDYWG | PPTFGQGTRL | | | | | | |
| | QGTDVTVSS | EIK (SEQ | | | | | | |
| | (SEQ ID | ID NO: | | | | | | |
| | NO: 1447) | 1359) | | | | | | |
| PMAB18 | QVQLVQSGAE | DIQMTQSPSS | YSFTT | MGIIA | ASGWV | QASRD | AASSL | QQSYS |
| | VKKPGASVKV | LSASVGDRVT | YYMH | PSGGS | Y | IKNYL | QS | TPPT |
| | SCKASGYSFT | ITCQASRDIK | (SEQ | TSYA | (SEQ | A | (SEQ | (SEQ |
| | TYYMHWVRQA | NYLAWYQQKP | ID | (SEQ | ID | (SEQ | ID | ID |
| | PGQGLEWMGI | GKAPKLLIYA | NO: | ID | NO: | ID | NO: | NO: |
| | IAPSGGSTSY | ASSLQSGVPS | 628) | NO: | 1489) | NO: | 1491) | 1492) |
| | AQKFQGRVTM | RFSGSGSGTD | | 1488) | | 1490) | | |
| | TRDTSTSTVY | FTLTISSLQP | | | | | | |
| | MELSSLRSED | EDFATYYCQQ | | | | | | |
| | TAVYYCASGW | SYSTPPTFGP | | | | | | |
| | VYWGQGTLVT | GTKVDIK | | | | | | |
| | VSS (SEQ | (SEQ ID | | | | | | |
| | ID NO: | NO: 1449) | | | | | | |
| | 1448) | | | | | | | |
| PMAB53 | EVQLLESGGG | DIVMTQSPDS | FTFSS | VSGIT | ARGRG | KSSQS | WASTR | QQYYT |
| | LVQPGGSLRL | LAVSLGERAT | YDMS | ISGGS | GSGWL | VLYSP | ES | TPPT |
| | SCAASGFTES | INCKSSQSVL | (SEQ | TYYA | DY | NNKNY | (SEQ | (SEQ |
| | SYDMSWVRQA | YSPNNKNYLA | ID | (SEQ | (SEQ | LA | ID | ID |
| | PGKGLEWVSG | WYQQKPGQPP | NO: | ID | ID | (SEQ | NO: | NO: |
| | ITISGGSTYY | KLLIYWASTR | 1487) | NO: | NO: | ID | 1485) | 1486) |
| | ADSVKGRFTI | ESGVPDRFSG | | 1482) | 1483) | NO: | | |
| | SRDNSKNTLY | SGSGTDFTLT | | | | 1484) | | |
| | LQMNSLRAED | ISSLQAEDVA | | | | | | |
| | TAVYYCARGR | VYYCQQYYTT | | | | | | |
| | GGSGWLDYWG | PPTFGQGTRL | | | | | | |
| | QGTTVTVSS | EIK (SEQ | | | | | | |
| | (SEQ ID | ID NO: | | | | | | |
| | NO: 1476) | 1359) | | | | | | |
| PMAB54 | EVQLVQSGAE | DIQMTQSPSS | YSFTS | MGIIN | ASGWV | RASQS | AASSL | QQSYS |
| | VKKPGASVKV | LSASVGDRVT | YYMH | PSGGS | Y | ISSYL | QS | TPPT |
| | SCKASGYSFT | ITCRASQSIS | (SEQ | TSYA | (SEQ | A | (SEQ | (SEQ |
| | SYYMHWVRQA | SYLAWYQQKP | ID | (SEQ | ID | (SEQ | ID | ID |
| | PGQGLEWMGI | GKAPKLLIYA | NO: | ID | NO: | ID | NO: | NO: |
| | INPSGGSTSY | ASSLQSGVPS | 766) | NO: | 1489) | NO: | 1491) | 1492) |
| | AQKFQGRVTM | RFSGSGSGTD | | 977) | | 1214) | | |
| | TRDTSTSTVY | FTLTISSLQP | | | | | | |
| | MELSSLRSED | EDFATYYCQQ | | | | | | |
| | TAVYYCASGW | SYSTPPTFGP | | | | | | |
| | VYWGQGTLVT | GTKVDIK | | | | | | |
| | VSS (SEQ | (SEQ ID | | | | | | |
| | ID NO: | NO: 1479) | | | | | | |
| | 1478) | | | | | | | |

In some embodiments, the therapeutic compound comprises one or more sequences, or a combination thereof, selected from the Table 13.

In some embodiments, the PD-1-MAdCAM antibody comprises an anti-MAdCAM scFv as provided for in the following table:

TABLE 14

| Clone | ScFv VH | Fab VL | | | | | | |
|---|---|---|---|---|---|---|---|---|
| (scFv) | Seq | Seq | HCDR1 | HCDR2 | HCDR3 | LCDR1 | LCDR2 | LCDR3 |
| PMAB1 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTES | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1495) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 591) | | | | | | | |
| PMAB2 | EVQLLESGGG | DIQMTQSPSS | FTFSS | SAISG | CARDR | RASQS | SSLQS | QQYKS |
| | LVQPGGSLRL | LSASVGDRVT | YWMH | SSGST | PYGYY | ISSSL | (SEQ | YPVT |
| | SCAASGFTFS | ITCRASQSIS | (SEQ | YYA | MDVW | A | ID | (SEQ |
| | SYWMHWVRQA | SSLAWYQQKP | ID | (SEQ | (SEQ | (SEQ | NO: | ID |
| | PGKGLEWVSA | GKAPKLLIYA | NO: | ID | ID | ID | 1497) | NO: |
| | ISGSSGSTYY | ASSLQSGVPS | 1499) | NO: | NO: | NO: | | 1498) |
| | ADSVKGRFTI | RFSGSGSGTD | | 1500) | 1501) | 1502) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYGYYMDVWG | GTKVEIK | | | | | | |
| | KGTTVTVSS | (SEQ ID | | | | | | |
| | (SEQ ID | NO: 1367) | | | | | | |
| | NO: 1347) | | | | | | | |
| PMAB3 | EVQLLESGGG | DIQMTQSPSS | FTFSS | SAISG | CARDR | RASQS | SSLQS | QQYKS |
| | LVQPGGSLRL | LSASVGDRVT | YWMH | SSGST | PYYYY | ISSSL | (SEQ | YPVT |
| | SCAASGFTES | ITCRASQSIS | (SEQ | YYA | MDVW | A | ID | (SEQ |
| | SYWMHWVRQA | SSLAWYQQKP | ID | (SEQ | (SEQ | (SEQ | NO: | ID |
| | PGKGLEWVSA | GKAPKLLIYA | NO: | ID | ID | ID | 1497) | NO: |
| | ISGSSGSTYY | ASSLQSGVPS | 1499) | NO: | NO: | NO: | | 1498) |
| | ADSVKGRFTI | RFSGSGSGTD | | 1500) | 1503) | 1502) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 1367) | | | | | | |
| | NO: 1439) | | | | | | | |
| PMAB5 | EVQLLESGGG | DIQMTQSPSS | FTFSS | SAISG | CARDR | RASQS | SSLQS | QQYKS |
| | LVQPGGSLRL | LSASVGDRVT | YWMH | SSGST | PYYYY | ISRSL | (SEQ | YPVT |
| | SCAASGFTFS | ITCRASQSIS | (SEQ | YYA | MDVW | A | ID | (SEQ |
| | SYWMHWVRQA | RSLAWYQQKP | ID | (SEQ | (SEQ | (SEQ | NO: | ID |
| | PGKGLEWVSA | GKAPKLLIYA | NO: | ID | ID | ID | 1497) | NO: |
| | ISGSSGSTYY | ASSLQSGVPS | 1499) | NO: | NO: | NO: | | 1498) |
| | ADSVKGRFTI | RFSGSGSGTD | | 1500) | 1503) | 1504) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 1363) | | | | | | |
| | NO: 1439) | | | | | | | |
| PMAB6 | EVQLLESGGG | DIQMTQSPSS | FTFSS | SAISG | CARDR | RASQS | SSLQS | QQYKS |
| | LVQPGGSLRL | LSASVGDRVT | YWMH | SGGST | PYGYY | ISSSL | (SEQ | YPVT |
| | SCAASGFTFS | ITCRASQSIS | (SEQ | YYA | MDVW | A | ID | (SEQ |
| | SYWMHWVRQA | SSLAWYQQKP | ID | (SEQ | (SEQ | (SEQ | NO: | ID |
| | PGKGLEWISA | GKAPKLLIYA | NO: | ID | ID | ID | 1497) | NO: |
| | ISGSGGSTYY | ASSLQSGVPS | 1499) | NO: | NO: | NO: | | 1498) |
| | ADSVKGRFTI | RFSGSGSGTD | | 1505) | 1501) | 1502) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYGYYMDVWG | GTKVEIK | | | | | | |
| | KGTTVTVSS | (SEQ ID | | | | | | |
| | (SEQ ID | NO: 1367) | | | | | | |
| | NO: 1440) | | | | | | | |
| PMAB7 | EVQLLESGGG | DIQMTQSPSS | FTFSS | SAISG | CARDR | RASQS | SSLQS | QQYKS |
| | LVQPGGSLRL | LSASVGDRVT | YWMH | SGGST | PYYYY | ISSSL | (SEQ | YPVT |
| | SCAASGFTFS | ITCRASQSIS | (SEQ | YYA | MDVW | A | ID | (SEQ |
| | SYWMHWVRQA | SSLAWYQQKP | ID | (SEQ | (SEQ | (SEQ | NO: | ID |
| | PGKGLEWISA | GKAPKLLIYA | NO: | ID | ID | ID | 1497) | NO: |
| | ISGSGGSTYY | ASSLQSGVPS | 1499) | NO: | NO: | NO: | | 1498) |
| | ADSVKGRFTI | RFSGSGSGTD | | 1505) | 1503) | 1502) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 1367) | | | | | | |
| | NO: 1441) | | | | | | | |
| PMAB8 | EVQLLESGGG | DIQMTQSPSS | FTFSS | SAISG | CARDR | RASQS | SSLQS | QQYKS |
| | LVQPGGSLRL | LSASVGDRVT | YWMH | SGGST | PYGYY | ISRSL | (SEQ | YPVT |
| | SCAASGFTFS | ITCRASQSIS | (SEQ | YYA | MDVW | A | ID | (SEQ |
| | SYWMHWVRQA | RSLAWYQQKP | ID | (SEQ | (SEQ | (SEQ | NO: | ID |
| | PGKGLEWISA | GKAPKLLIYA | NO: | ID | ID | ID | 1497) | NO: |
| | ISGSGGSTYY | ASSLQSGVPS | 1499) | NO: | NO: | NO: | | 1498) |
| | ADSVKGRFTI | RFSGSGSGTD | | 1505) | 1501) | 1504) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYGYYMDVWG | GTKVEIK | | | | | | |
| | KGTTVTVSS | (SEQ ID | | | | | | |
| | (SEQ ID | NO: 1363) | | | | | | |
| | NO: 1440) | | | | | | | |
| PMAB9 | EVQLLESGGG | DIQMTQSPSS | FTFSS | SAISG | CARDR | RASQS | SSLQS | QQYKS |
| | LVQPGGSLRL | LSASVGDRVT | YWMH | SGGST | PYYYY | ISRSL | (SEQ | YPVT |
| | SCAASGFTFS | ITCRASQSIS | (SEQ | YYA | MDVW | A | ID | (SEQ |
| | SYWMHWVRQA | RSLAWYQQKP | ID | (SEQ | (SEQ | (SEQ | NO: | ID |
| | PGKGLEWISA | GKAPKLLIYA | NO: | ID | ID | ID | 1497) | NO: |
| | ISGSGGSTYY | ASSLQSGVPS | 1499) | NO: | NO: | NO: | | 1498) |
| | ADSVKGRFTI | RFSGSGSGTD | | 1505) | 1503) | 1504) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 1363) | | | | | | |
| | NO: 1441) | | | | | | | |
| PMAB10 | EVQLLESGGG | DIQMTQSPSS | FTFSS | SAISG | CARDR | RASQS | SSLQS | QQYKS |
| | LVQPGGSLRL | LSASVGDRVT | YWMH | SGGST | PYGYY | ISSSL | (SEQ | YPVT |
| | SCAASGFTES | ITCRASQSIS | (SEQ | YYA | MDVW | A | ID | (SEQ |
| | SYWMHWVRQA | SSLAWYQQKP | ID | (SEQ | (SEQ | (SEQ | NO: | ID |
| | PGKGLEWVSA | GKAPKLLIYA | NO: | ID | ID | ID | 1497) | NO: |
| | ISGSGGSTYY | ASSLQSGVPS | 1499) | NO: | NO: | NO: | | 1498) |
| | ADSVKGRFTI | RFSGSGSGTD | | 1505) | 1501) | 1502) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYGYYMDVWG | GTKVEIK | | | | | | |
| | KGTTVTVSS | (SEQ ID | | | | | | |
| | (SEQ ID | NO: 1367) | | | | | | |
| | NO: 1442) | | | | | | | |
| PMAB11 | EVQLLESGGG | DIQMTQSPSS | FTFSS | SAISG | CARDR | RASQS | SSLQS | QQYKS |
| | LVQPGGSLRL | LSASVGDRVT | YWMH | SGGST | PYYYY | ISSSL | (SEQ | YPVT |
| | SCAASGFTES | ITCRASQSIS | (SEQ | YYA | MDVW | A | ID | (SEQ |
| | SYWMHWVRQA | SSLAWYQQKP | ID | (SEQ | (SEQ | (SEQ | NO: | ID |
| | PGKGLEWVSA | GKAPKLLIYA | NO: | ID | ID | ID | 1497) | NO: |
| | ISGSGGSTYY | ASSLQSGVPS | 1499) | NO: | NO: | NO: | | 1498) |
| | ADSVKGRFTI | RFSGSGSGTD | | 1505) | 1503) | 1502) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 1367) | | | | | | |
| | NO: 1395) | | | | | | | |
| PMAB12 | EVQLLESGGG | DIQMTQSPSS | FTFSS | SAISG | CARDR | RASQS | SSLQS | QQYKS |
| | LVQPGGSLRL | LSASVGDRVT | YWMH | SGGST | PYGYY | ISRSL | (SEQ | YPVT |
| | SCAASGFTES | ITCRASQSIS | (SEQ | YYA | MDVW | A | ID | (SEQ |
| | SYWMHWVRQA | RSLAWYQQKP | ID | (SEQ | (SEQ | (SEQ | NO: | ID |
| | PGKGLEWVSA | GKAPKLLIYA | NO: | ID | ID | ID | 1497) | NO: |
| | ISGSGGSTYY | ASSLQSGVPS | 1499) | NO: | NO: | NO: | | 1498) |
| | ADSVKGRFTI | RFSGSGSGTD | | 1505) | 1501) | 1504) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYGYYMDVWG | GTKVEIK | | | | | | |
| | KGTTVTVSS | (SEQ ID | | | | | | |
| | (SEQ ID | NO: 1363) | | | | | | |
| | NO: 1442) | | | | | | | |
| PMAB13 | EVQLLESGGG | DIQMTQSPSS | FTFSS | SAISG | CARDR | RASQS | SSLQS | QQYKS |
| | LVQPGGSLRL | LSASVGDRVT | YWMH | SGGST | PYYYY | ISRSL | (SEQ | YPVT |
| | SCAASGFTFS | ITCRASQSIS | (SEQ | YYA | MDVW | A | ID | (SEQ |
| | SYWMHWVRQA | RSLAWYQQKP | ID | (SEQ | (SEQ | (SEQ | NO: | ID |
| | PGKGLEWVSA | GKAPKLLIYA | NO: | ID | ID | ID | 1497) | NO: |
| | ISGSGGSTYY | ASSLQSGVPS | 1499) | NO: | NO: | NO: | | 1498) |
| | ADSVKGRFTI | RFSGSGSGTD | | 1505) | 1503) | 1504) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 1363) | | | | | | |
| | NO: 1395) | | | | | | | |
| PMAB14 | EVQLLESGGG | DIQMTQSPSS | FTFSS | SYISG | CARDR | RASQS | SSLQS | QQYKS |
| | LVQPGGSLRL | LSASVGDRVT | YWMH | SGGYT | PYGYY | ISSSL | (SEQ | YPVT |
| | SCAASGFTES | ITCRASQSIS | (SEQ | NYA | MDVW | A | ID | (SEQ |
| | SYWMHWVRQA | SSLAWYQQKP | ID | (SEQ | (SEQ | (SEQ | NO: | ID |
| | PGKGLEWVSY | GKAPKLLIYA | NO: | ID | ID | ID | 1497) | NO: |
| | ISGSGGYTNY | ASSLQSGVPS | 1499) | NO: | NO: | NO: | | 1498) |
| | ADSVKGRFTI | RFSGSGSGTD | | 1506) | 1501) | 1502) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYGYYMDVWG | GTKVEIK | | | | | | |
| | KGTTVTVSS | (SEQ ID | | | | | | |
| | (SEQ ID | NO: 1367) | | | | | | |
| | NO: 1443) | | | | | | | |
| PMAB15 | EVQLLESGGG | DIQMTQSPSS | FTFSS | SAISG | CARDR | RASQS | SSLQS | QQYKS |
| | LVQPGGSLRL | LSASVGDRVT | YWMH | SGGST | PYYYD | ISSSL | (SEQ | YPVT |
| | SCAASGFTFS | ITCRASQSIS | (SEQ | YYA | MDVW | A | ID | (SEQ |
| | SYWMHWVRQA | SSLAWYQQKP | ID | (SEQ | (SEQ | (SEQ | NO: | ID |
| | PGKGLEWVSA | GKAPKLLIYA | NO: | ID | ID | ID | 1497) | NO: |
| | ISGSGGSTYY | ASSLQSGVPS | 1499) | NO: | NO: | NO: | | 1498) |
| | ADSVKGRFTI | RFSGSGSGTD | | 1505) | 1507) | 1502) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYDMDVWG | GTKVEIK | | | | | | |
| | KGTTVTVSS | (SEQ ID | | | | | | |
| | (SEQ ID | NO: 1367) | | | | | | |
| | NO: 1444) | | | | | | | |
| PMAB16 | EVQLLESGGG | DIQMTQSPSS | FTFSS | SYISG | CARDR | RASQS | SSLQS | QQYKS |
| | LVQPGGSLRL | LSASVGDRVT | YWMH | SGGYT | PYYYD | ISSSL | (SEQ | YPVT |
| | SCAASGFTFS | ITCRASQSIS | (SEQ | NYA | MDVW | A | ID | (SEQ |
| | SYWMHWVRQA | SSLAWYQQKP | ID | (SEQ | (SEQ | (SEQ | NO: | ID |
| | PGKGLEWVSY | GKAPKLLIYA | NO: | ID | ID | ID | 1497) | NO: |
| | ISGSGGYTNY | ASSLQSGVPS | 1499) | NO: | NO: | NO: | | 1498) |
| | ADSVKGRFTI | RFSGSGSGTD | | 1506) | 1507) | 1502) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYDMDVWG | GTKVEIK | | | | | | |
| | KGTTVTVSS | (SEQ ID | | | | | | |
| | (SEQ ID | NO: 1367) | | | | | | |
| | NO: 1445) | | | | | | | |
| PMAB19 | EVQLLESGGG | DIQMTQSPSS | FTFSS | SYISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTFS | ITCRASQSVS | (SEQ | NYA | MDVW | A | ID | (SEQ |
| | SFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1508) | NO: | NO: | NO: | | 1498) |
| | ADSVKGRFTI | RFSGSGSGTD | | 1494) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1353) | | | | | | | |
| PMAB20 | EVQLLESGGG | DIQMTQSPSS | FTFSD | SYISG | CARDR | RASQS | SSLQS | QQYKS |
| | LVQPGGSLRL | LSASVGDRVT | YWMH | DSGYT | PYYYY | VSRSL | (SEQ | YPVT |
| | SCAASGFTFS | ITCRASQSVS | (SEQ | NYA | MDVW | A | ID | (SEQ |
| | DYWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1509) | NO: | NO: | NO: | | 1498) |
| | ADSVKGRFTI | RFSGSGSGTD | | 1494) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1391) | | | | | | | |
| PMAB21 | EVQLLESGGG | DIQMTQSPSS | FTFSD | SYISG | CARDR | RASQS | SSLQS | QQYKS |
| | LVQPGGSLRL | LSASVGDRVT | FAMH | DSGYT | PYYYY | VSRSL | (SEQ | YPVT |
| | SCAASGFTFS | ITCRASQSVS | (SEQ | NYA | MDVW | A | ID | (SEQ |
| | DFAMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1510) | NO: | NO: | NO: | | 1498) |
| | ADSVKGRFTI | RFSGSGSGTD | | 1494) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1450) | | | | | | | |
| PMAB22 | EVQLLESGGG | DIQMTQSPSS | FTFSD | SYISG | CARDR | RASQS | SSLQS | QQYKS |
| | LVQPGGSLRL | LSASVGDRVT | FWMS | DSGYT | PYYYY | VSRSL | (SEQ | YPVT |
| | SCAASGFTFS | ITCRASQSVS | (SEQ | NYA | MDVW | A | ID | (SEQ |
| | DFWMSWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1511) | NO: | NO: | NO: | | 1498) |
| | ADSVKGRFTI | RFSGSGSGTD | | 1494) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1451) | | | | | | | |
| PMAB23 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWVSY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1452) | | | | | | | |
| PMAB24 | EVQLLESGGG | DIQMTQSPSS | FTFSD | SAISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTES | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISA | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1512) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1453) | | | | | | | |
| PMAB25 | EVQLLESGGG | DIQMTQSPSS | FTFSD | SYISG | CARDR | RASQS | SSLQS | QQYKS |
| | LVQPGGSLRL | LSASVGDRVT | FWMH | SSGYT | PYYYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGSSGYTNY | ASSLQSGVPS | 1493) | NO: | NO: | NO: | | 1498) |
| | ADSVKGRFTI | RFSGSGSGTD | | 1513) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1454) | | | | | | | |
| PMAB26 | EVQLLESGGG | DIQMTQSPSS | FTFSD | SYISG | CARDR | RASQS | SSLQS | QQYKS |
| | LVQPGGSLRL | LSASVGDRVT | FWMH | DGGYT | PYYYY | VSRSL | (SEQ | YPVT |
| | SCAASGFTES | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDGGYTNY | ASSLQSGVPS | 1493) | NO: | NO: | NO: | | 1498) |
| | ADSVKGRFTI | RFSGSGSGTD | | 1514) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1455) | | | | | | | |
| PMAB27 | EVQLLESGGG | DIQMTQSPSS | FTFSD | SYISG | CARDR | RASQS | SSLQS | QQYKS |
| | LVQPGGSLRL | LSASVGDRVT | FWMH | DSGST | PYYYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGSTNY | ASSLQSGVPS | 1493) | NO: | NO: | NO: | | 1498) |
| | ADSVKGRFTI | RFSGSGSGTD | | 1515) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1456) | | | | | | | |
| PMAB28 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTES | ITCRASQSVS | (SEQ | YYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTYY | ASSLQSGVPS | 1493) | NO: | NO: | NO: | | 1498) |
| | ADSVKGRFTI | RFSGSGSGTD | | 1516) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1457) | | | | | | | |
| PMAB29 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | AASVKGRFTI | RFSGSGSGTD | | 1494) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1458) | | | | | | | |
| PMAB30 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTES | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | AQSVKGRFTI | RFSGSGSGTD | | 1494) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1459) | | | | | | | |
| PMAB31 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1503) | 1496) | | |
| | SRDASKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1460) | | | | | | | |
| PMAB32 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1503) | 1496) | | |
| | SRDQSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1379) | | | | | | | |
| PMAB33 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1503) | 1496) | | |
| | SRDNSKNTLY | FTLTISSLQP | | | | | | |
| | LQMASLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1461) | | | | | | | |
| PMAB34 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1503) | 1496) | | |
| | SRDNSKNTLY | FTLTISSLQP | | | | | | |
| | LQMGSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1462) | | | | | | | |
| PMAB35 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTES | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1503) | 1496) | | |
| | SRDNSKNTLY | FTLTISSLQP | | | | | | |
| | LQMQSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 592) | | | | | | |
| | NO: 1463) | | | | | | | |
| PMAB36 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | ISRSL | (SEQ | YPVT |
| | SCAASGFTFS | ITCRASQSIS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1503) | 1504) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 1363) | | | | | | |
| | NO: 591) | | | | | | | |
| PMAB37 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSSSL | (SEQ | YPVT |
| | SCAASGFTES | ITCRASQSVS | (SEQ | NYA | MDVW | A | ID | (SEQ |
| | DFWMHWVRQA | SSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1503) | 1517) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 1364) | | | | | | |
| | NO: 591) | | | | | | | |
| PMAB38 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRYL | (SEQ | YPVT |
| | SCAASGFTFS | ITCRASQSVS | (SEQ | NYA | MDVW | A | ID | (SEQ |
| | DFWMHWVRQA | RYLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1503) | 1518) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 1365) | | | | | | |
| | NO: 591) | | | | | | | |
| PMAB39 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | YPVT |
| | SCAASGFTES | ITCRASQSVS | (SEQ | NYA | MDVW | N | ID | (SEQ |
| | DFWMHWVRQA | RSLNWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1503) | 1519) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYYMDVWG | GTKVEIK | | | | | | |
| | KGTTVTVSS | (SEQ ID | | | | | | |
| | (SEQ ID | NO: 1366) | | | | | | |
| | NO: 591) | | | | | | | |
| PMAB40 | EVQLLESGGG | DIQMTQSPSS | FTFSD | SYISG | CARDR | RASQS | SSLQS | QQSKS |
| | LVQPGGSLRL | LSASVGDRVT | FWMH | DSGYT | PYYYY | VSRSL | (SEQ | YPVT |
| | SCAASGFTES | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1520) |
| | ADSVKGRFTI | RFSGSGSGTD | | 1494) | 1503) | 1496) | | |
| | SRDNSKNTLY | FTLTISSLQP | | | | | | |
| | LQMNSLRAED | EDFATYYCQQ | | | | | | |
| | TAVYYCARDR | SKSYPVTFGQ | | | | | | |
| | PYYYYMDVWG | GTKVEIK | | | | | | |
| | KGTTVTVSS | (SEQ ID | | | | | | |
| | (SEQ ID | NO: 1464) | | | | | | |
| | NO: 591) | | | | | | | |
| PMAB41 | EVQLLESGGG | DIQMTQSPSS | FTFSD | SYISG | CARDR | RASQS | SSLQS | QQYYS |
| | LVQPGGSLRL | LSASVGDRVT | FWMH | DSGYT | PYYYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1521) |
| | ADSVKGRFTI | RFSGSGSGTD | | 1494) | 1503) | 1496) | | |
| | SRDNSKNTLY | FTLTISSLQP | | | | | | |
| | LQMNSLRAED | EDFATYYCQQ | | | | | | |
| | TAVYYCARDR | YYSYPVTFGQ | | | | | | |
| | PYYYYMDVWG | GTKVEIK | | | | | | |
| | KGTTVTVSS | (SEQ ID | | | | | | |
| | (SEQ ID | NO: 1465) | | | | | | |
| | NO: 591) | | | | | | | |
| PMAB42 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | TPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1522) |
| | ADSVKGRFTI | RFSGSGSGTD | | 1494) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSTPVTFGQ | | | | | | |
| | PYYYYMDVWG | GTKVEIK | | | | | | |
| | KGTTVTVSS | (SEQ ID | | | | | | |
| | (SEQ ID | NO: 1466) | | | | | | |
| | NO: 591) | | | | | | | |
| PMAB43 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYY | VSRSL | (SEQ | YPRT |
| | SCAASGFTES | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1523) |
| | ADSVKGRFTI | RFSGSGSGTD | | 1494) | 1503) | 1496) | | |
| | SRDNSKNTLY | FTLTISSLQP | | | | | | |
| | LQMNSLRAED | EDFATYYCQQ | | | | | | |
| | TAVYYCARDR | YKSYPRTFGQ | | | | | | |
| | PYYYYMDVWG | GTKVEIK | | | | | | |
| | KGTTVTVSS | (SEQ ID | | | | | | |
| | (SEQ ID | NO: 1467) | | | | | | |
| | NO: 591) | | | | | | | |
| PMAB44 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PTYYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1524) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PTYYYMDVWG | GTKVEIK | | | | | | |
| | KGTTVTVSS | (SEQ ID | | | | | | |
| | (SEQ ID | NO: 592) | | | | | | |
| | NO: 1390) | | | | | | | |
| PMAB45 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYDYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1525) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYDYYMDVWG | GTKVEIK | | | | | | |
| | KGTTVTVSS | (SEQ ID | | | | | | |
| | (SEQ ID | NO: 592) | | | | | | |
| | NO: 1468) | | | | | | | |
| PMAB46 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYGYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1501) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYGYYMDVWG | GTKVEIK | | | | | | |
| | KGTTVTVSS | (SEQ ID | | | | | | |
| | (SEQ ID | NO: 592) | | | | | | |
| | NO: 1469) | | | | | | | |
| PMAB47 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYNYY | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1526) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYNYYMDVWG | GTKVEIK | | | | | | |
| | KGTTVTVSS | (SEQ ID | | | | | | |
| | (SEQ ID | NO: 592) | | | | | | |
| | NO: 1470) | | | | | | | |
| PMAB48 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYSYY | VSRSL | (SEQ | YPVT |
| | SCAASGETES | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1527) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYSYYMDVWG | GTKVEIK | | | | | | |
| | KGTTVTVSS | (SEQ ID | | | | | | |
| | (SEQ ID | NO: 592) | | | | | | |
| | NO: 1471) | | | | | | | |
| PMAB49 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYD | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1507) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYDMDVWG | GTKVEIK | | | | | | |
| | KGTTVTVSS | (SEQ ID | | | | | | |
| | (SEQ ID | NO: 592) | | | | | | |
| | NO: 1472) | | | | | | | |
| PMAB50 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYE | VSRSL | (SEQ | YPVT |
| | SCAASGFTES | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1528) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYEMDVWG | GTKVEIK | | | | | | |
| | KGTTVTVSS | (SEQ ID | | | | | | |
| | (SEQ ID | NO: 592) | | | | | | |
| | NO: 1473) | | | | | | | |
| PMAB51 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYK | VSRSL | (SEQ | YPVT |
| | SCAASGFTES | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1529) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYKMDVWG | GTKVEIK | | | | | | |
| | KGTTVTVSS | (SEQ ID | | | | | | |
| | (SEQ ID | NO: 592) | | | | | | |
| | NO: 1474) | | | | | | | |
| PMAB52 | EVQLLESGGG | DIQMTQSPSS | FTFSD | SYISG | CARDR | RASQS | SSLQS | QQYKS |
| | LVQPGGSLRL | LSASVGDRVT | FWMH | DSGYT | PYYYS | VSRSL | (SEQ | YPVT |
| | SCAASGFTFS | ITCRASQSVS | (SEQ | NYA | MDVW | A | ID | (SEQ |
| | DFWMHWVRQA | RSLAWYQQKP | ID | (SEQ | (SEQ | (SEQ | NO: | ID |
| | PGKGLEWISY | GKAPKLLIYA | NO: | ID | ID | ID | 1497) | NO: |
| | ISGDSGYTNY | ASSLQSGVPS | 1493) | NO: | NO: | NO: | | 1498) |
| | ADSVKGRFTI | RFSGSGSGTD | | 1494) | 1530) | 1496) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYSMDVWG | GTKVEIK | | | | | | |
| | KGTTVTVSS | (SEQ ID | | | | | | |
| | (SEQ ID | NO: 592) | | | | | | |
| | NO: 1475) | | | | | | | |
| PMAB53 | EVQLLESGGG | DIQMTQSPSS | FTFSS | SYISG | CARDR | RASQS | SSLQS | QQYKS |
| | LVQPGGSLRL | LSASVGDRVT | YWMH | SGGYT | PYYYD | ISSSL | (SEQ | YPVT |
| | SCAASGFTFS | ITCRASQSIS | (SEQ | NYA | IDVW | A | ID | (SEQ |
| | SYWMHWVRQA | SSLAWYQQKP | ID | (SEQ | (SEQ | (SEQ | NO: | ID |
| | PGKGLEWVSY | GKAPKLLIYA | NO: | ID | ID | ID | 1497) | NO: |
| | ISGSGGYTNY | ASSLQSGVPS | 1499) | NO: | NO: | NO: | | 1498) |
| | ADSVKGRFTI | RFSGSGSGTD | | 1506) | 1531) | 1502) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYDIDVWG | GTKVEIK | | | | | | |
| | KGTTVTVSS | (SEQ ID | | | | | | |
| | (SEQ ID | | | | | | | |
| | NO: 1477) | NO: 1367) | | | | | | |
| PMAB55 | EVQLLESGGG | DIQMTQSPSS | FTFSS | SYISG | CARDR | RASQS | SSLQS | QQYKS |
| | LVQPGGSLRL | LSASVGDRVT | YWMH | SGGYT | PYYYD | ISSSL | (SEQ | YPVT |
| | SCAASGFTFS | ITCRASQSIS | (SEQ | NYA | LDVW | A | ID | (SEQ |
| | SYWMHWVRQA | SSLAWYQQKP | ID | (SEQ | (SEQ | (SEQ | NO: | ID |
| | PGKGLEWVSY | GKAPKLLIYA | NO: | ID | ID | ID | 1497) | NO: |
| | ISGSGGYTNY | ASSLQSGVPS | 1499) | NO: | NO: | NO: | | 1498) |
| | ADSVKGRFTI | RFSGSGSGTD | | 1506) | 1532) | 1502) | | |
| | SRDNSKNTLY | FTLTISSLQP | | | | | | |
| | LQMNSLRAED | EDFATYYCQQ | | | | | | |
| | TAVYYCARDR | YKSYPVTFGQ | | | | | | |
| | PYYYDLDVWG | GTKVEIK | | | | | | |
| | KGTTVTVSS | (SEQ ID | | | | | | |
| | (SEQ ID | NO: 1367) | | | | | | |
| | NO: 1480) | | | | | | | |

In some embodiments, the therapeutic compound comprises one or more sequences, or a combination thereof, selected from the Table 14.

In some embodiments, the therapeutic a Fab PD-1 antibody fused via a linker to a scFv MAdCAM antibody. In some embodiments, the Fab PD-1 antibody is fused to a IgG1 constant domain, wherein said IgG1 constant domain is fused to scFv MAdCAM antibody via a Fc-scFv linker. In some embodiment the scFv MAdCAM antibody comprises an internal scFv linker. In some embodiments, the linker is a peptide linker. In some embodiments, the peptide linker is a glycine/serine linker as provided herein.

In some embodiments, the PD-1-MAdCAM antibody comprises one or more sequences as shown in Table 12. In some embodiments, the MAdCAM antibody comprises a combination of one or more sequence as shown in Table 12. In some embodiments, the anti-PD-1 antibody is in the Fab format and the anti-MAdCAM antibody is in a scFV format as illustrated in Table 12. In some embodiments, the Fab portion of the antibody comprises a CDR1 from any one of clones PMAB1-54 of Table 13, a CDR2 from any one of clones PMAB1-54 of Table 13, and a CDR3 from any one of clones PMAB1-54 of Table 13. In some embodiments, the Fab portion of the antibody comprises a LCDR1 from any one of clones PMAB1-54 of Table 13, a LCDR2 from any one of clones PMAB1-54 of Table 13, and a LCDR3 from any one of clones PMAB1-54 of Table 13. In some embodiments, the scFv portion of the antibody comprises a CDR1 from any one of clones PMAB1-55 of Table 14, a CDR2 from any one of clones PMAB1-55 of Table 14, and a CDR3 from any one of clones PMAB1-55 of Table 14. In some embodiments, the scFc portion of the antibody comprises a LCDR1 from any one of clones PMAB1-55 of Table 14, a LCDR2 from any one of clones PMAB1-55 of Table 14, and a LCDR3 from any one of clones PMAB1-55 of Table 14. In some embodiments, the amino acid residues of the CDRs shown above contain mutations. In some embodiments, the CDRs contain 0, 1, 2, 3, 4, 5, 6, 7, 8, 9, or 10 substitutions or mutations. In some embodiments, the substitution is a conservative substitution.

In some embodiments, the PD-1-MAdCAM antibody has a VH region selected from any one of clones PMAB1-77 of Table 12 and a VL region selected from any one of clones PMAB1-77 as set forth in of Table 12. In some embodiments, the antibody comprises a Fab CDR1 from any one of clones PMAB1-54 of Table 13, a Fab CDR2 from any one of clones PMAB1-54 of Table 13, and a Fab CDR3 from any one of clones PMAB1-54 of Table 13, a scFv CDR1 from any one of clones PMAB1-55 of Table 14, a Fab CDR2 from any one of clones PMAB1-55 of Table 14, and a Fab CDR3 from any one of clones PMAB1-55 of Table 14.

In some embodiments, the variable heavy chain has a Fab HCDR1, HCDR2, and a HCDR3 as set forth for PMAB1 in Table 13. In some embodiments, the variable heavy chain has a Fab HCDR1, HCDR2, and a HCDR3 as set forth for PMAB15 in Table 13. In some embodiments, the variable heavy chain has a Fab HCDR1, HCDR2, and a HCDR3 as set forth for PMAB17 in Table 13. In some embodiments, the variable heavy chain has a Fab HCDR1, HCDR2, and a HCDR3 as set forth for PMAB18 in Table 13. In some embodiments, the variable heavy chain has a Fab HCDR1, HCDR2, and a HCDR3 as set forth for PMAB53 in Table 13. In some embodiments, the variable heavy chain has a Fab HCDR1, HCDR2, and a HCDR3 as set forth for PMAB54 in Table 13.

In some embodiments, the variable light chain has a Fab LCDR1, LCDR2, and a LCDR3 as set forth for PMAB1 in Table 13. In some embodiments, the variable light chain has a Fab LCDR1, LCDR2, and a LCDR3 as set forth for PMAB15 in Table 13. In some embodiments, the variable light chain has a Fab LCDR1, LCDR2, and a LCDR3 as set forth for PMAB17 in Table 13. In some embodiments, the variable light chain has a Fab LCDR1, LCDR2, and a LCDR3 as set forth for PMAB18 in Table 13. In some embodiments, the variable light chain has a Fab LCDR1, LCDR2, and a LCDR3 as set forth for PMAB53 in Table 13. In some embodiments, the variable light chain has a Fab LCDR1, LCDR2, and a LCDR3 as set forth for PMAB54 in Table 13.

In some embodiments, the variable heavy chain has a scFv HCDR1, HCDR2, and a HCDR3 as set forth for PMAB1 in Table 14. In some embodiments, the variable heavy chain has a scFv HCDR1, HCDR2, and a HCDR3 as set forth for PMAB2 in Table 14. In some embodiments, the variable heavy chain has a scFv HCDR1, HCDR2, and a HCDR3 as set forth for PMAB3 in Table 14. In some embodiments, the variable heavy chain has a scFv HCDR1, HCDR2, and a HCDR3 as set forth for PMAB5 in Table 14. In some embodiments, the variable heavy chain has a scFv HCDR1, HCDR2, and a HCDR3 as set forth for PMAB6 in Table 14. In some embodiments, the variable heavy chain has a scFv HCDR1, HCDR2, and a HCDR3 as set forth for PMAB7 in Table 14. In some embodiments, the variable heavy chain has a scFv HCDR1, HCDR2, and a HCDR3 as set forth for PMAB8 in Table 14. In some embodiments, the variable heavy chain has a scFv HCDR1, HCDR2, and a HCDR3 as set forth for PMAB9 in Table 14. In some embodiments, the variable heavy chain has a scFv HCDR1, HCDR2, and a HCDR3 as set forth for PMAB10 in Table 14. In some embodiments, the variable heavy chain has a scFv HCDR1, HCDR2, and a HCDR3 as set forth for PMAB11 in Table 14. In some embodiments, the variable heavy chain has a scFv HCDR1, HCDR2, and a HCDR3 as set forth for PMAB12 in Table 14. In some embodiments, the variable heavy chain has a scFv HCDR1, HCDR2, and a HCDR3 as set forth for PMAB13 in Table 14. In some embodiments, the variable heavy chain has a scFv HCDR1, HCDR2, and a HCDR3 as set forth for PMAB14 in Table 14. In some embodiments, the variable heavy chain has a scFv HCDR1, HCDR2, and a HCDR3 as set forth for PMAB15 in Table 14. In some embodiments, the variable heavy chain has a scFv HCDR1, HCDR2, and a HCDR3 as set forth for PMAB16 in Table 14. In some embodiments, the variable heavy chain has a scFv HCDR1, HCDR2, and a HCDR3 as set forth for PMAB19 in Table 14. In some embodiments, the variable heavy chain has a scFv HCDR1, HCDR2, and a HCDR3 as set forth for PMAB20 in Table 14. In some embodiments, the variable heavy chain has a scFv HCDR1, HCDR2, and a HCDR3 as set forth for PMAB21 in Table 14. In some embodiments, the variable heavy chain has a scFv HCDR1, HCDR2, and a HCDR3 as set forth for PMAB22 in Table 14. In some embodiments, the variable heavy chain has a scFv HCDR1, HCDR2, and a HCDR3 as set forth for PMAB23 in Table 14. In some embodiments, the variable heavy chain has a scFv HCDR1, HCDR2, and a HCDR3 as set forth for PMAB24 in Table 14. In some embodiments, the variable heavy chain has a scFv HCDR1, HCDR2, and a HCDR3 as set forth for PMAB25 in Table 14. In some embodiments, the variable heavy chain has a scFv HCDR1, HCDR2, and a HCDR3 as set forth for PMAB26 in Table 14. In some embodiments, the variable heavy chain has a scFv HCDR1, HCDR2, and a HCDR3 as set forth for PMAB27 in Table 14. In some embodiments, the variable heavy chain has a scFv HCDR1, HCDR2, and a HCDR3 as set forth for PMAB28 in Table 14. In some embodiments, the variable heavy chain has a scFv HCDR1, HCDR2, and a HCDR3 as set forth for PMAB29 in Table 14. In some embodiments, the variable heavy chain has a scFv HCDR1, HCDR2, and a HCDR3 as set forth for PMAB30 in Table 14. In some embodiments, the variable heavy chain has a scFv HCDR1, HCDR2, and a HCDR3 as set forth for PMAB31 in Table 14. In some embodiments, the variable heavy chain has a scFv HCDR1, HCDR2, and a HCDR3 as set forth for PMAB32 in Table 14. In some embodiments, the variable heavy chain has a scFv HCDR1, HCDR2, and a HCDR3 as set forth for PMAB33 in Table 14. In some embodiments, the variable heavy chain has a scFv HCDR1, HCDR2, and a HCDR3 as set forth for PMAB34 in Table 14. In some embodiments, the variable heavy chain has a scFv HCDR1, HCDR2, and a HCDR3 as set forth for PMAB35 in Table 14. In some embodiments, the variable heavy chain has a scFv HCDR1, HCDR2, and a HCDR3 as set forth for PMAB36 in Table 14. In some embodiments, the variable heavy chain has a scFv HCDR1, HCDR2, and a HCDR3 as set forth for PMAB37 in Table 14. In some embodiments, the variable heavy chain has a scFv HCDR1, HCDR2, and a HCDR3 as set forth for PMAB38 in Table 14. In some embodiments, the variable heavy chain has a scFv HCDR1, HCDR2, and a HCDR3 as set forth for PMAB39 in Table 14. In some embodiments, the variable heavy chain has a scFv HCDR1, HCDR2, and a HCDR3 as set forth for PMAB40 in Table 14. In some embodiments, the variable heavy chain has a scFv HCDR1, HCDR2, and a HCDR3 as set forth for PMAB41 in Table 14. In some embodiments, the variable heavy chain has a scFv HCDR1, HCDR2, and a HCDR3 as set forth for PMAB42 in Table 14. In some embodiments, the variable heavy chain has a scFv HCDR1, HCDR2, and a HCDR3 as set forth for PMAB43 in Table 14. In some embodiments, the variable heavy chain has a scFv HCDR1, HCDR2, and a HCDR3 as set forth for PMAB44 in Table 14. In some embodiments, the variable heavy chain has a scFv HCDR1, HCDR2, and a HCDR3 as set forth for PMAB45 in Table 14. In some embodiments, the variable heavy chain has a scFv HCDR1, HCDR2, and a HCDR3 as set forth for PMAB46 in Table 14. In some embodiments, the variable heavy chain has a scFv HCDR1, HCDR2, and a HCDR3 as set forth for PMAB47 in Table 14. In some embodiments, the variable heavy chain has a scFv HCDR1, HCDR2, and a HCDR3 as set forth for PMAB48 in Table 14. In some embodiments, the variable heavy chain has a scFv HCDR1, HCDR2, and a HCDR3 as set forth for PMAB49 in Table 14. In some embodiments, the variable heavy chain has a scFv HCDR1, HCDR2, and a HCDR3 as set forth for PMAB50 in Table 14. In some embodiments, the variable heavy chain has a scFv HCDR1, HCDR2, and a HCDR3 as set forth for PMAB51 in Table 14. In some embodiments, the variable heavy chain has a scFv HCDR1, HCDR2, and a HCDR3 as set forth for PMAB52 in Table 14. In some embodiments, the variable heavy chain has a scFv HCDR1, HCDR2, and a HCDR3 as set forth for PMAB53 in Table 14. In some embodiments, the variable heavy chain has a scFv HCDR1, HCDR2, and a HCDR3 as set forth for PMAB55 in Table 14.

In some embodiments, the variable light chain has a scFv LCDR1, LCDR2, and a LCDR3 as set forth for PMAB1 in Table 14. In some embodiments, the variable light chain has a scFv LCDR1, LCDR2, and a LCDR3 as set forth for PMAB2 in Table 14. In some embodiments, the variable light chain has a scFv LCDR1, LCDR2, and a LCDR3 as set forth for PMAB3 in Table 14. In some embodiments, the variable light chain has a scFv LCDR1, LCDR2, and a LCDR3 as set forth for PMAB5 in Table 14. In some embodiments, the variable light chain has a scFv LCDR1, LCDR2, and a LCDR3 as set forth for PMAB6 in Table 14. In some embodiments, the variable light chain has a scFv LCDR1, LCDR2, and a LCDR3 as set forth for PMAB7 in Table 14. In some embodiments, the variable light chain has a scFv LCDR1, LCDR2, and a LCDR3 as set forth for PMAB8 in Table 14. In some embodiments, the variable light chain has a scFv LCDR1, LCDR2, and a LCDR3 as set forth for PMAB9 in Table 14. In some embodiments, the variable light chain has a scFv LCDR1, LCDR2, and a LCDR3 as set forth for PMAB10 in Table 14. In some embodiments, the variable light chain has a scFv LCDR1, LCDR2, and a LCDR3 as set forth for PMAB11 in Table 14. In some embodiments, the variable light chain has a scFv LCDR1, LCDR2, and a LCDR3 as set forth for PMAB12 in Table 14. In some embodiments, the variable light chain has a scFv LCDR1, LCDR2, and a LCDR3 as set forth for PMAB13 in Table 14. In some embodiments, the variable light chain has a scFv LCDR1, LCDR2, and a LCDR3 as set forth for PMAB14 in Table 14. In some embodiments, the variable light chain has a scFv LCDR1, LCDR2, and a LCDR3 as set forth for PMAB15 in Table 14. In some embodiments, the variable light chain has a scFv LCDR1, LCDR2, and a LCDR3 as set forth for PMAB16 in Table 14. In some embodiments, the variable light chain has a scFv LCDR1, LCDR2, and a LCDR3 as set forth for PMAB19 in Table 14. In some embodiments, the variable light chain has a scFv LCDR1, LCDR2, and a LCDR3 as set forth for PMAB20 in Table 14. In some embodiments, the variable light chain has a scFv LCDR1, LCDR2, and a LCDR3 as set forth for PMAB21 in Table 14. In some embodiments, the variable light chain has a scFv LCDR1, LCDR2, and a LCDR3 as set forth for PMAB22 in Table 14. In some embodiments, the variable light chain has a scFv LCDR1, LCDR2, and a LCDR3 as set forth for PMAB23 in Table 14. In some embodiments, the variable light chain has a scFv LCDR1, LCDR2, and a LCDR3 as set forth for PMAB24 in Table 14. In some embodiments, the variable light chain has a scFv LCDR1, LCDR2, and a LCDR3 as set forth for PMAB25 in Table 14. In some embodiments, the variable light chain has a scFv LCDR1, LCDR2, and a LCDR3 as set forth for PMAB26 in Table 14. In some embodiments, the variable light chain has a scFv LCDR1, LCDR2, and a LCDR3 as set forth for PMAB27 in Table 14. In some embodiments, the variable light chain has a scFv LCDR1, LCDR2, and a LCDR3 as set forth for PMAB28 in Table 14. In some embodiments, the variable light chain has a scFv LCDR1, LCDR2, and a LCDR3 as set forth for PMAB29 in Table 14. In some embodiments, the variable light chain has a scFv LCDR1, LCDR2, and a LCDR3 as set forth for PMAB30 in Table 14. In some embodiments, the variable light chain has a scFv LCDR1, LCDR2, and a LCDR3 as set forth for PMAB31 in Table 14. In some embodiments, the variable light chain has a scFv LCDR1, LCDR2, and a LCDR3 as set forth for PMAB32 in Table 14. In some embodiments, the variable light chain has a scFv LCDR1, LCDR2, and a LCDR3 as set forth for PMAB33 in Table 14. In some embodiments, the variable light chain has a scFv LCDR1, LCDR2, and a LCDR3 as set forth for PMAB34 in Table 14. In some embodiments, the variable light chain has a scFv LCDR1, LCDR2, and a LCDR3 as set forth for PMAB35 in Table 14. In some embodiments, the variable light chain has a scFv LCDR1, LCDR2, and a LCDR3 as set forth for PMAB36 in Table 14. In some embodiments, the variable light chain has a scFv LCDR1, LCDR2, and a LCDR3 as set forth for PMAB37 in Table 14. In some embodiments, the variable light chain has a scFv LCDR1, LCDR2, and a LCDR3 as set forth for PMAB38 in Table 14. In some embodiments, the variable light chain has a scFv LCDR1, LCDR2, and a LCDR3 as set forth for PMAB39 in Table 14. In some embodiments, the variable light chain has a scFv LCDR1, LCDR2, and a LCDR3 as set forth for PMAB40 in Table 14. In some embodiments, the variable light chain has a scFv LCDR1, LCDR2, and a LCDR3 as set forth for PMAB41 in Table 14. In some embodiments, the variable light chain has a scFv LCDR1, LCDR2, and a LCDR3 as set forth for PMAB42 in Table 14. In some embodiments, the variable light chain has a scFv LCDR1, LCDR2, and a LCDR3 as set forth for PMAB43 in Table 14. In some embodiments, the variable light chain has a scFv LCDR1, LCDR2, and a LCDR3 as set forth for PMAB44 in Table 14. In some embodiments, the variable light chain has a scFv LCDR1, LCDR2, and a LCDR3 as set forth for PMAB45 in Table 14. In some embodiments, the variable light chain has a scFv LCDR1, LCDR2, and a LCDR3 as set forth for PMAB46 in Table 14. In some embodiments, the variable light chain has a scFv LCDR1, LCDR2, and a LCDR3 as set forth for PMAB47 in Table 14. In some embodiments, the variable light chain has a scFv LCDR1, LCDR2, and a LCDR3 as set forth for PMAB48 in Table 14. In some embodiments, the variable light chain has a scFv LCDR1, LCDR2, and a LCDR3 as set forth for PMAB49 in Table 14. In some embodiments, the variable light chain has a scFv LCDR1, LCDR2, and a LCDR3 as set forth for PMAB50 in Table 14. In some embodiments, the variable light chain has a scFv LCDR1, LCDR2, and a LCDR3 as set forth for PMAB51 in Table 14. In some embodiments, the variable light chain has a scFv LCDR1, LCDR2, and a LCDR3 as set forth for PMAB52 in Table 14. In some embodiments, the variable light chain has a scFv LCDR1, LCDR2, and a LCDR3 as set forth for PMAB53 in Table 14. In some embodiments, the variable light chain has a scFv LCDR1, LCDR2, and a LCDR3 as set forth for PMAB55 in Table 14.

In some embodiments, the CDRS are swapped for one another. For example, the Fab HCDR1 of clone PMAB1 can be substituted for the Fab HCDR1 of clone PMAB2, or vice versa. This CDR swapping can be done for any of the Fab HCDRs of the clones provided herein (e.g., HCDR1 for HCDR1; HCDR2 for HCDR2; or HCDR3 for HCDR3) or the Fab LCDRs (e.g., LCDR1 for LCDR1; LCDR2 for LCDR2; or LCDR3 for LCDR3). Furthermore, the CDR swapping can be done for any of the scFv HCDRs of the clones provided herein (e.g., HCDR1 for HCDR1; HCDR2 for HCDR2; or HCDR3 for HCDR3) or the scFv LCDRs (e.g., LCDR1 for LCDR1; LCDR2 for LCDR2; or LCDR3 for LCDR3). Therefore, in some embodiments, the antibody comprises a Fab HCDR1 as set forth in any of PMAB1-54 of Table 13, a HCDR2 as set forth in any of PMAB1-54 of Table 13, a HCDR3 as set forth in any of PMAB1-54 of Table 13, a LCDR1 as set forth in any of PMAB1-54 of Table 13, a LCDR2 as set forth in any of PMAB1-54 of Table 13, a LCDR3 as set forth in any of PMAB1-54 of Table 13, or a variant of any of the foregoing. In some embodiments, the antibody comprises a scFv HCDR1 as set forth in any of PMAB1-55 of Table 14, a HCDR2 as set forth in any of PMAB1-55 of Table 14, a HCDR3 as set forth in any of PMAB1-55 of Table 14, a LCDR1 as set forth in any of PMAB1-55 of Table 14, a LCDR2 as set forth in any of PMAB1-55 of Table 14, a LCDR3 as set forth in any of PMAB1-55 of Table 14, or a variant of any of the foregoing.

In some embodiments, the amino acid residues of the CDRs shown above contain mutations. In some embodiments, the CDRs contain 0, 1, 2, 3, 4, 5, 6, 7, 8, 9, or 10 substitutions or mutations. In some embodiments, the substitution is a conservative substitution.

In some embodiments, the anti-MAdCAM antibody, or antigen binding fragment thereof, comprises i) a heavy chain variable region comprising heavy chain CDR1, CDR2, and CDR3 sequences, wherein the heavy chain CDR1 sequence has an amino acid sequence having at least one amino acid substitution relative to SEQ ID NO: 1499; the heavy chain CDR2 has an amino acid sequence having at least one amino acid substitution relative to SEQ ID NO: 1506; and the heavy chain CDR3 has an amino acid sequence having at least one amino acid substitution relative to SEQ ID NOs: 1507, 1531, or 1532; and (ii) a light chain variable region comprising light chain CDR1, CDR2, and CDR3 sequences, wherein the light chain CDR1 sequence has an amino acid sequence having at least one amino acid substitution relative to SEQ ID NO: 1502; the light chain CDR2 has an amino acid sequence having at least one amino acid substitution relative to SEQ ID NO: 1497; and the light chain CDR3 has an amino acid sequence having at least one amino acid substitution relative to SEQ ID NO: 1498.

In some embodiments, the anti-MAdCAM antibody, or antigen binding fragment thereof, comprises i) a heavy chain variable region comprising heavy chain CDR1, CDR2, and CDR3 sequences, wherein the heavy chain CDR1 sequence has an amino acid sequence having one or zero amino acid substitution relative to SEQ ID NO: 1499; the heavy chain CDR2 has an amino acid sequence having one or zero amino acid substitution relative to SEQ ID NO: 1506; and the heavy chain CDR3 has an amino acid sequence having one or zero amino acid substitution relative to SEQ ID NOs: 1507, 1531, or 1532; and (ii) a light chain variable region comprising light chain CDR1, CDR2, and CDR3 sequences, wherein the light chain CDR1 sequence has an amino acid sequence having one or zero amino acid substitution relative to SEQ ID NO: 1502; the light chain CDR2 has an amino acid sequence having one or zero amino acid substitution relative to SEQ ID NO: 1497; and the light chain CDR3 has an amino acid sequence having one or zero amino acid substitution relative to SEQ ID NO: 1498.

In some embodiments, the anti-MAdCAM antibody, or antigen binding fragment thereof, heavy chain variable region comprises a heavy chain CDR1 comprising the amino acid sequence of SEQ ID NO: 1499, a heavy chain CDR2 comprising the amino acid sequence of SEQ ID NO: 1506, and a heavy chain CDR3 comprising the amino acid sequence of SEQ ID NO: 1507. In some embodiments, the anti-MAdCAM antibody, or antigen binding fragment thereof, heavy chain variable region comprises a heavy chain CDR1 comprising the amino acid sequence of SEQ ID NO: 1499, a heavy chain CDR2 comprising the amino acid sequence of SEQ ID NO: 1506, and a heavy chain CDR3 comprising the amino acid sequence of SEQ ID NO: 1531. In some embodiments, the anti-MAdCAM antibody, or antigen binding fragment thereof, heavy chain variable region comprises a heavy chain CDR1 comprising the amino acid sequence of SEQ ID NO: 1499, a heavy chain CDR2 comprising the amino acid sequence of SEQ ID NO: 1506, and a heavy chain CDR3 comprising the amino acid sequence of SEQ ID NO: 1532. In some embodiments, the anti-MAdCAM antibody, or antigen binding fragment thereof, light chain variable region comprises a light chain CDR1 comprising the amino acid sequence of SEQ ID NO: 1502, a light chain CDR2 comprising the amino acid sequence of SEQ ID NO: 1497, and a light chain CDR3 comprising the amino acid sequence of SEQ ID NO: 1498.

In some embodiments, the antibody that binds to PD-1 comprises i) a heavy chain variable region comprising heavy chain CDR1, CDR2, and CDR3 sequences, wherein the heavy chain CDR1 sequence has an amino acid sequence having at least one amino acid substitution relative to SEQ ID NO: 1481, or 1487; the heavy chain CDR2 has an amino acid sequence having at least one amino acid substitution relative to SEQ ID NO: 1482, or 1488; and the heavy chain CDR3 has an amino acid sequence having at least one amino acid substitution relative to SEQ ID NOs: 1483, or 1489; and (ii) a light chain variable region comprising light chain CDR1, CDR2, and CDR3 sequences, wherein the light chain CDR1 sequence has an amino acid sequence having at least one amino acid substitution relative to SEQ ID NO: 1484, 1490, or 1214; the light chain CDR2 has an amino acid sequence having at least one amino acid substitution relative to SEQ ID NO: 1485, or 1491; and the light chain CDR3 has an amino acid sequence having at least one amino acid substitution relative to SEQ ID NO: 1486, or 1492.

In some embodiments, a composition comprising an effector molecule linked to an antibody, or antigen binding fragment thereof, comprises:
a) the antibody in an scFv orientation comprising i) a heavy chain variable region comprising heavy chain CDR1, CDR2, and CDR3 sequences, wherein the heavy chain CDR1 sequence has an amino acid sequence having one or zero amino acid substitution relative to SEQ ID NO: 1499; the heavy chain CDR2 has an amino acid sequence having one or zero amino acid substitution relative to SEQ ID NO: 1506; and the heavy chain CDR3 has an amino acid sequence having one or zero amino acid substitution relative to SEQ ID NOs: 1507, 1531, or 1532; and (ii) a light chain variable region comprising light chain CDR1, CDR2, and CDR3 sequences, wherein the light chain CDR1 sequence has an amino acid sequence having one or zero amino acid substitution relative to SEQ ID NO: 1502; the light chain CDR2 has an amino acid sequence having one or zero amino acid substitution relative to SEQ ID NO: 1497; and the light chain CDR3 has an amino acid sequence having one or zero amino acid substitution relative to SEQ ID NO: 1498; and
b) the effector molecule in a Fab orientation comprising i) a heavy chain variable region comprising heavy chain CDR1, CDR2, and CDR3 sequences, wherein the heavy chain CDR1 sequence has an amino acid sequence having one or zero amino acid substitution relative to SEQ ID NO: 1481, or 1487; the heavy chain CDR2 has an amino acid sequence having one or zero amino acid substitution relative to SEQ ID NO: 1482, or 1488; and the heavy chain CDR3 has an amino acid sequence having one or zero amino acid substitution relative to SEQ ID NOs: 1483, or 1489; and (ii) a light chain variable region comprising light chain CDR1, CDR2, and CDR3 sequences, wherein the light chain CDR1 sequence has an amino acid sequence having one or zero amino acid substitution relative to SEQ ID NO: 1484, 1490, or 1214; the light chain CDR2 has an amino acid sequence having one or zero amino acid substitution relative to SEQ ID NO: 1485, or 1491; and the light chain CDR3 has an amino acid sequence having one or zero amino acid substitution relative to SEQ ID NO: 1486, or 1492.

In some embodiments, the composition comprising an effector molecule linked to an antibody, or antigen binding fragment thereof, comprises:
a) the antibody in an scFv orientation comprising i) a heavy chain variable region comprising heavy chain CDR1, CDR2, and CDR3 sequences, wherein the heavy chain CDR1 sequence has an amino acid sequence of SEQ ID NO: 1499; the heavy chain CDR2 has an amino acid sequence of SEQ ID NO: 1506; and the heavy chain CDR3 has an amino acid sequence of SEQ ID NOs: 1507, 1531, or 1532; and (ii) a light chain variable region comprising light chain CDR1, CDR2, and CDR3 sequences, wherein the light chain CDR1 sequence has an amino acid sequence of SEQ ID NO: 1502; the light chain CDR2 has an amino acid sequence of SEQ ID NO: 1497; and the light chain CDR3 has an amino acid sequence of SEQ ID NO: 1498; and
b) the effector molecule in a Fab orientation comprising i) a heavy chain variable region comprising heavy chain CDR1, CDR2, and CDR3 sequences, wherein the heavy chain CDR1 sequence has an amino acid sequence of SEQ ID NO: 1481, or 1487; the heavy chain CDR2 has an amino acid sequence of SEQ ID NO: 1482, or 1488; and the heavy chain CDR3 has an amino acid sequence of SEQ ID NOs: 1483, or 1489; and (ii) a light chain variable region comprising light chain CDR1, CDR2, and CDR3 sequences, wherein the light chain CDR1 sequence has an amino acid sequence of SEQ ID NO: 1484, 1490, or 1214; the light chain CDR2 has an amino acid sequence of SEQ ID NO: 1485, or 1491; and the light chain CDR3 has an amino acid sequence of SEQ ID NO: 1486, or 1492.

In some embodiments, the composition comprising an effector molecule linked to an antibody, or antigen binding fragment thereof, comprises:
a) the antibody in an scFv orientation comprising i) a heavy chain variable region comprising heavy chain CDR1, CDR2, and CDR3 sequences, wherein the heavy chain CDR1 sequence has the amino acid sequence of SEQ ID NO: 1499; the heavy chain CDR2 has the amino acid sequence of SEQ ID NO: 1506; and the heavy chain CDR3 has the amino acid sequence of SEQ ID NOs: 1507, or variants of any of the foregoing; and (ii) a light chain variable region comprising light chain CDR1, CDR2, and CDR3 sequences, wherein the light chain CDR1 sequence has the amino acid sequence of SEQ ID NO: 1502; the light chain CDR2 has the amino acid sequence of SEQ ID NO: 1497; and the light chain CDR3 has the amino acid sequence of SEQ ID NO: 1498, or variants of any of the foregoing; and
b) the effector molecule in a Fab orientation comprising i) a heavy chain variable region comprising heavy chain CDR1, CDR2, and CDR3 sequences, wherein the heavy chain CDR1 sequence has the amino acid sequence of SEQ ID NO: 1487; the heavy chain CDR2 has the amino acid sequence of SEQ ID NO: 1488; and the heavy chain CDR3 has the amino acid sequence of SEQ ID NO: 1489, or variants of any of the foregoing; and (ii) a light chain variable region comprising light chain CDR1, CDR2, and CDR3 sequences, wherein the light chain CDR1 sequence has the amino acid sequence of SEQ ID NO: 1490; the light chain CDR2 has the amino acid sequence of SEQ ID NO: 1491; and the light chain CDR3 has the amino acid sequence of SEQ ID NO: 1492, or variants of any of the foregoing.

In some embodiments, the composition comprising an effector molecule linked to an antibody, or antigen binding fragment thereof, comprises:
a) the antibody in an scFv orientation comprising i) a heavy chain variable region comprising heavy chain CDR1, CDR2, and CDR3 sequences, wherein the heavy chain CDR1 sequence has the amino acid sequence of SEQ ID NO: 1499; the heavy chain CDR2 has the amino acid sequence of SEQ ID NO: 1506; and the heavy chain CDR3 has the amino acid sequence of SEQ ID NOs: 1507, or variants of any of the foregoing; and (ii) a light chain variable region comprising light chain CDR1, CDR2, and CDR3 sequences, wherein the light chain CDR1 sequence has the amino acid sequence of SEQ ID NO: 1502; the light chain CDR2 has the amino acid sequence of SEQ ID NO: 1497; and the light chain CDR3 has the amino acid sequence of SEQ ID NO: 1498, or variants of any of the foregoing; and
b) the effector molecule in a Fab orientation comprising i) a heavy chain variable region comprising heavy chain CDR1, CDR2, and CDR3 sequences, wherein the heavy chain CDR1 sequence has the amino acid sequence of SEQ ID NO: 1487; the heavy chain CDR2 has the amino acid sequence of SEQ ID NO: 1482; and the heavy chain CDR3 has the amino acid sequence of SEQ ID NO: 1483, or variants of any of the foregoing; and (ii) a light chain variable region comprising light chain CDR1, CDR2, and CDR3 sequences, wherein the light chain CDR1 sequence has the amino acid sequence of SEQ ID NO: 1484; the light chain CDR2 has the amino acid sequence of SEQ ID NO: 1485; and the light chain CDR3 has the amino acid sequence of SEQ ID NO: 1486, or variants of any of the foregoing.

In some embodiments, the VH comprises a sequence as set forth in Table 12. In some embodiments, the VK comprises a sequence as set forth in Table 12. In some embodiments, the Ab comprises a VH and a VK as set forth for the clones in Table 12. In some embodiments, the VH and VK are linked by a linker. In some embodiments, the linker is a peptide linker as provided for herein. In some embodiments, the peptide linker is the linker as provided for in Table 12.

In some embodiments, the anti-MAdCAM antibody, or antigen binding fragment thereof, heavy chain variable region comprises an amino acid sequence having at least 90% identity to the amino acid sequence of SEQ ID NO: 1445, 1477, or 1480 and the anti-MAdCAM antibody light chain variable region comprises an amino acid sequence having at least 90% identity to the amino acid sequence of SEQ ID NO: 1367. In some embodiments, the anti-MAdCAM antibody, or antigen binding fragment thereof, heavy chain variable region comprises an amino acid sequence of SEQ ID NOs: 1445, 1477, or 1480 and the anti-MAdCAM antibody light chain variable region comprises an amino acid of SEQ ID NO: 1367.

In some embodiments, the anti-MAdCAM antibody, or antigen binding fragment thereof, comprises a light chain variable region comprising an amino acid sequence having at least 90% identity to an amino acid sequence selected from SEQ ID NOs: 592, 1360, 1361, 1362, 1363, 1364, 1365, 1366, 1367, 1368, 1369, 1370, 1371, 1372, 1373, 1374, 1375, 1376, 1380, 1381, 1382, 1383, 1384, 1385, 1386, 1464, 1465, 1466, 1467, or 1543; and a heavy chain variable region comprising an amino acid sequence having at least 90% identity to an amino acid sequence selected from SEQ ID NOs: 591, 1346, 1347, 1348, 1349, 1350, 1351, 1352, 1353, 1354, 1355, 1356, 1357, 1358, 1377, 1378, 1379, 1387, 1388, 1389, 1390, 1391, 1392, 1393, 1394, 1395, 1396, 1397, 1398, 1439, 1440, 1441, 1442, 1443, 1444, 1445, 1450, 1451, 1452, 1453, 1454, 1455, 1456, 1457, 1458, 1459, 1460, 1461, 1462, 1463, 1469, 1470, 1471, 1472, 1473, 1474, 1475, 1477, 1480, 1542, 1544, or 1545.

In some embodiments, the antibody that binds to PD-1 comprises a light chain variable region comprising an amino acid sequence having at least 90% identity to an amino acid sequence selected from SEQ ID NOs: 1359, 1449, or 1479; and the variable heavy chain comprising an amino acid sequence having at least 90% identity to an amino acid sequence selected from SEQ ID NOs: 1438, 1446, 1447, 1448, 1476, or 1478.

In some embodiments, the composition comprising an effector molecule linked to an antibody, or antigen binding fragment thereof, comprises: a) the antibody in an scFv orientation comprising a light chain variable region comprising an amino acid sequence having at least 95% identity to an amino acid sequence selected from SEQ ID NOs: 1367; and a heavy chain variable region comprising an amino acid sequence having at least 95% identity to an amino acid sequence selected from SEQ ID NOs: 1445; and b) the effector molecule in a Fab orientation comprising a light chain variable region comprising an amino acid sequence having at least 95% identity to an amino acid sequence selected from SEQ ID NOs: 1359, 1449, or 1479; and a heavy chain variable region comprising an amino acid sequence having at least 95% identity to an amino acid sequence selected from SEQ ID NOs: 1438, 1446, 1447, 1448, 1476, or 1478.

In some embodiments, the composition comprising an effector molecule linked to an antibody, or antigen binding fragment thereof, comprises:
a) the antibody in an scFv orientation comprising a light chain variable region comprising an amino acid sequence of SEQ ID NOs: 1367; and a heavy chain variable region comprising an amino acid sequence of SEQ ID NOs: 1445; and
b) the effector molecule in a Fab orientation comprising a light chain variable region comprising an amino acid sequence of SEQ ID NOs: 1359, 1449, or 1479; and a heavy chain variable region comprising an amino acid sequence of SEQ ID NOs: 1438, 1446, 1447, 1448, 1476, or 1478.

In some embodiments, the composition comprising an effector molecule linked to an antibody, or antigen binding fragment thereof, comprises:
a) the antibody in an scFv orientation comprising a light chain variable region comprising an amino acid sequence of SEQ ID NOs: 1367; and a heavy chain variable region comprising an amino acid sequence of SEQ ID NOs: 1445; and
b) the effector molecule in a Fab orientation comprising a light chain variable region comprising an amino acid sequence of SEQ ID NOs: 1449; and a heavy chain variable region comprising an amino acid sequence of SEQ ID NOs: 1448.

In some embodiments, the composition comprising an effector molecule linked to an antibody, or antigen binding fragment thereof, comprises:
a) the antibody in an scFv orientation comprising a light chain variable region comprising an amino acid sequence of SEQ ID NOs: 1367; and a heavy chain variable region comprising an amino acid sequence of SEQ ID NOs: 1445; and
b) the effector molecule in a Fab orientation comprising a light chain variable region comprising an amino acid sequence of SEQ ID NOs: 1359; and a heavy chain variable region comprising an amino acid sequence of SEQ ID NOs: 1476.

### Pharmaceutical Compositions and Kits

In another aspect, the present embodiments provide compositions, e.g., pharmaceutically acceptable compositions, which include a therapeutic compound described herein, formulated together with a pharmaceutically acceptable carrier. As used herein, “pharmaceutically acceptable carrier” includes any and all solvents, dispersion media, isotonic and absorption delaying agents, and the like that are physiologically compatible.

The carrier can be suitable for intravenous, intramuscular, subcutaneous, parenteral, rectal, local, ophthalmic, topical, spinal or epidermal administration (e.g. by injection or infusion). As used herein, the term “carrier” means a diluent, adjuvant, or excipient with which a compound is administered. In some embodiments, pharmaceutical carriers can also be liquids, such as water and oils, including those of petroleum, animal, vegetable or synthetic origin, such as peanut oil, soybean oil, mineral oil, sesame oil and the like. The pharmaceutical carriers can also be saline, gum acacia, gelatin, starch paste, talc, keratin, colloidal silica, urea, and the like. In addition, auxiliary, stabilizing, thickening, lubricating and coloring agents can be used. The carriers can be used in pharmaceutical compositions comprising the therapeutic compounds provided for herein.

The compositions and compounds of the embodiments provided for herein may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Typical compositions are in the form of injectable or infusible solutions. In some embodiments, the mode of administration is parenteral (e.g., intravenous, subcutaneous, intraperitoneal, intramuscular). In some embodiments, the therapeutic molecule is administered by intravenous infusion or injection. In another embodiment, the therapeutic molecule is administered by intramuscular or subcutaneous injection. In another embodiment, the therapeutic molecule is administered locally, e.g., by injection, or topical application, to a target site. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions typically should be sterile and stable under the conditions of manufacture and storage. The composition can be formulated as a solution, microemulsion, dispersion, liposome, or other ordered structure suitable to high therapeutic molecule concentration. Sterile injectable solutions can be prepared by incorporating the active compound (i.e., therapeutic molecule) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

In certain embodiments, a therapeutic compound can be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound by other than parenteral administration, it may be necessary to coat the compound with, or co-administer the compound with, a material to prevent its inactivation. Therapeutic compositions can also be administered with medical devices known in the art.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage.

Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of a therapeutic compound is 0.1-30 mg/kg, more preferably 1-25 mg/kg. Dosages and therapeutic regimens of the therapeutic compound can be determined by a skilled artisan. In certain embodiments, the therapeutic compound is administered by injection (e.g., subcutaneously or intravenously) at a dose of about 1 to 40 mg/kg, e.g., 1 to 30 mg/kg, e.g., about 5 to 25 mg/kg, about 10 to 20 mg/kg, about 1 to 5 mg/kg, 1 to 10 mg/kg, 5 to 15 mg/kg, 10 to 20 mg/kg, 15 to 25 mg/kg, or about 3 mg/kg. The dosing schedule can vary from e.g., once a week to once every 2, 3, or 4 weeks. In one embodiment, the therapeutic compound is administered at a dose from about 10 to 20 mg/kg every other week. The therapeutic compound can be administered by intravenous infusion at a rate of more than 20 mg/min, e.g., 20-40 mg/min, and typically greater than or equal to 40 mg/min to reach a dose of about 35 to 440 mg/m², typically about 70 to 310 mg/m², and more typically, about 110 to 130 mg/m². In embodiments, the infusion rate of about 110 to 130 mg/m² achieves a level of about 3 mg/kg. In other embodiments, the therapeutic compound can be administered by intravenous infusion at a rate of less than 10 mg/min, e.g., less than or equal to 5 mg/min to reach a dose of about 1 to 100 mg/m², e.g., about 5 to 50 mg/m², about 7 to 25 mg/m², or, about 10 mg/m². In some embodiments, the therapeutic compound is infused over a period of about 30 min. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions may include a “therapeutically effective amount” or a “prophylactically effective amount” of a therapeutic molecule. A “therapeutically effective amount” refers to an amount effective, at dosages and for periods of time necessary, to achieve the desired therapeutic result. A therapeutically effective amount of a therapeutic molecule may vary according to factors such as the disease state, age, sex, and weight of the individual, and the ability of the therapeutic compound to elicit a desired response in the individual. A therapeutically effective amount is also one in which any toxic or detrimental effects of a therapeutic molecule is outweighed by the therapeutically beneficial effects. A “therapeutically effective dosage” preferably inhibits a measurable parameter, e.g., immune attack at least about 20%, more preferably by at least about 40%, even more preferably by at least about 60%, and still more preferably by at least about 80% relative to untreated subjects. The ability of a compound to inhibit a measurable parameter, e.g., immune attack, can be evaluated in an animal model system predictive of efficacy in transplant rejection or autoimmune disorders. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the embodiments is a kit comprising a therapeutic compound described herein. The kit can include one or more other elements including: instructions for use; other reagents, e.g., a label, a therapeutic agent, or an agent useful for chelating, or otherwise coupling, a therapeutic molecule to a label or other therapeutic agent, or a radioprotective composition; devices or other materials for preparing a therapeutic molecule for administration; pharmaceutically acceptable carriers; and devices or other materials for administration to a subject.

In some embodiments, embodiments provided herein also include, but are not limited to:

1. A method of treating or preventing Type 1 diabetes comprising administering to a subject in need thereof, an anti-PD-1 agonist antibody linked to an anti-MAdCAM antibody, or antigen binding fragment thereof, wherein the anti-MAdCAM antibody, or antigen binding fragment thereof, comprises:
i) a heavy chain variable region comprising heavy chain CDR1, CDR2, and CDR3 sequences, wherein the heavy chain CDR1 sequence has an amino acid sequence having at least one amino acid substitution relative to SEQ ID NO: 1499; the heavy chain CDR2 has an amino acid sequence having at least one amino acid substitution relative to SEQ ID NO: 1506; and the heavy chain CDR3 has an amino acid sequence having at least one amino acid substitution relative to SEQ ID NOs: 1507, 1531, or 1532; and
(ii) a light chain variable region comprising light chain CDR1, CDR2, and CDR3 sequences, wherein the light chain CDR1 sequence has an amino acid sequence having at least one amino acid substitution relative to SEQ ID NO: 1502; the light chain CDR2 has an amino acid sequence having at least one amino acid substitution relative to SEQ ID NO: 1497; and the light chain CDR3 has an amino acid sequence having at least one amino acid substitution relative to SEQ ID NO: 1498.
   2. The method of embodiment 1, wherein the subject has Type 1 diabetes or is a subject at risk of developing Type 1 diabetes.
   3. The method of any one of embodiments 1 or 2, wherein the anti-MAdCAM antibody, or antigen binding fragment thereof, comprises:
i) a heavy chain variable region comprising heavy chain CDR1, CDR2, and CDR3 sequences, wherein the heavy chain CDR1 sequence has an amino acid sequence having one or zero amino acid substitution relative to SEQ ID NO: 1499; the heavy chain CDR2 has an amino acid sequence having one or zero amino acid substitution relative to SEQ ID NO: 1506; and the heavy chain CDR3 has an amino acid sequence having one or zero amino acid substitution relative to SEQ ID NOs: 1507, 1531, or 1532; and
(ii) a light chain variable region comprising light chain CDR1, CDR2, and CDR3 sequences, wherein the light chain CDR1 sequence has an amino acid sequence having one or zero amino acid substitution relative to SEQ ID NO: 1502; the light chain CDR2 has an amino acid sequence having one or zero amino acid substitution relative to SEQ ID NO: 1497; and the light chain CDR3 has an amino acid sequence having one or zero amino acid substitution relative to SEQ ID NO: 1498.
   4. The method of embodiment 1, wherein the anti-MAdCAM antibody, or antigen binding fragment thereof, heavy chain variable region comprises a heavy chain CDR1 comprising the amino acid sequence of SEQ ID NO: 1499, a heavy chain CDR2 comprising the amino acid sequence of SEQ ID NO: 1506, and a heavy chain CDR3 comprising the amino acid sequence of SEQ ID NO: 1507.
   5. The method of embodiment 1, wherein the anti-MAdCAM antibody, or antigen binding fragment thereof, heavy chain variable region comprises a heavy chain CDR1 comprising the amino acid sequence of SEQ ID NO: 1499, a heavy chain CDR2 comprising the amino acid sequence of SEQ ID NO: 1506, and a heavy chain CDR3 comprising the amino acid sequence of SEQ ID NO: 1531.
   6. The method of embodiment 1, wherein the anti-MAdCAM antibody, or antigen binding fragment thereof, heavy chain variable region comprises a heavy chain CDR1 comprising the amino acid sequence of SEQ ID NO: 1499, a heavy chain CDR2 comprising the amino acid sequence of SEQ ID NO: 1506, and a heavy chain CDR3 comprising the amino acid sequence of SEQ ID NO: 1532.
   7. The method of any one of embodiments 1-6, wherein the anti-MAdCAM antibody, or antigen binding fragment thereof, light chain variable region comprises a light chain CDR1 comprising the amino acid sequence of SEQ ID NO: 1502, a light chain CDR2 comprising the amino acid sequence of SEQ ID NO: 1497, and a light chain CDR3 comprising the amino acid sequence of SEQ ID NO: 1498.
   8. The method of embodiment 1, wherein the anti-MAdCAM antibody, or antigen binding fragment thereof, heavy chain variable region comprises an amino acid sequence having at least 90% identity to the amino acid sequence of SEQ ID NO: 1445, 1477, or 1480 and the anti-MAdCAM antibody light chain variable region comprises an amino acid sequence having at least 90% identity to the amino acid sequence of SEQ ID NO: 1367.
   9. The method of any one of embodiments 1-8, wherein the anti-MAdCAM antibody, or antigen binding fragment thereof, heavy chain variable region comprises an amino acid sequence of SEQ ID NOs: 1445, 1477, or 1480 and the anti-MAdCAM antibody light chain variable region comprises an amino acid of SEQ ID NO: 1367.
   10. The method of any one of embodiments 1-9, wherein the anti-MAdCAM antibody, or antigen binding fragment thereof, heavy chain variable region and the light chain variable region are in a Fab, or an scFv format.
   11. The method of any one of embodiments 1-10, wherein the anti-MAdCAM antibody, or antigen binding fragment thereof, heavy chain variable region and the light variable chain region are linked with a peptide linker.
   12. The method of embodiment 11, wherein the peptide linker is a glycine/serine linker.
   13. A method of treating Type 1 diabetes comprising administering to a subject in need thereof, an effector molecule linked to an antibody, or antigen binding fragment thereof, wherein the antibody comprises:
a light chain variable region comprising an amino acid sequence having at least 90% identity to an amino acid sequence selected from SEQ ID NOs: 592, 1360, 1361, 1362, 1363, 1364, 1365, 1366, 1367, 1368, 1369, 1370, 1371, 1372, 1373, 1374, 1375, 1376, 1380, 1381, 1382, 1383, 1384, 1385, 1386, 1464, 1465, 1466, 1467, or 1543; and
a heavy chain variable region comprising an amino acid sequence having at least 90% identity to an amino acid sequence selected from SEQ ID NOs: 591, 1346, 1347, 1348, 1349, 1350, 1351, 1352, 1353, 1354, 1355, 1356, 1357, 1358, 1377, 1378, 1379, 1387, 1388, 1389, 1390, 1391, 1392, 1393, 1394, 1395, 1396, 1397, 1398, 1439, 1440, 1441, 1442, 1443, 1444, 1445, 1450, 1451, 1452, 1453, 1454, 1455, 1456, 1457, 1458, 1459, 1460, 1461, 1462, 1463, 1469, 1470, 1471, 1472, 1473, 1474, 1475, 1477, 1480, 1542, 1544, or 1545.
   14. The method of embodiment 13, wherein the effector molecule is a PD-1 agonist.
   15. The method of embodiment 15, wherein the PD-1 agonist is an antibody that binds to PD-1.
   16. The method of embodiment 15, wherein the antibody that binds to PD-1 comprises:
i) a heavy chain variable region comprising heavy chain CDR1, CDR2, and CDR3 sequences, wherein the heavy chain CDR1 sequence has an amino acid sequence having at least one amino acid substitution relative to SEQ ID NO: 1481, or 1487; the heavy chain CDR2 has an amino acid sequence having at least one amino acid substitution relative to SEQ ID NO: 1482, or 1488; and the heavy chain CDR3 has an amino acid sequence having at least one amino acid substitution relative to SEQ ID NOs: 1483, or 1489; and
(ii) a light chain variable region comprising light chain CDR1, CDR2, and CDR3 sequences, wherein the light chain CDR1 sequence has an amino acid sequence having at least one amino acid substitution relative to SEQ ID NO: 1484, 1490, or 1214; the light chain CDR2 has an amino acid sequence having at least one amino acid substitution relative to SEQ ID NO: 1485, or 1491; and the light chain CDR3 has an amino acid sequence having at least one amino acid substitution relative to SEQ ID NO: 1486, or 1492.
   17. The method of embodiment 16, wherein the antibody that binds to PD-1 comprises:
a light chain variable region comprising an amino acid sequence having at least 90% identity to an amino acid sequence selected from SEQ ID NOs: 1359, 1449, or 1479; and
the variable heavy chain comprising an amino acid sequence having at least 90% identity to an amino acid sequence selected from SEQ ID NOs: 1438, 1446, 1447, 1448, 1476, or 1478.
   18. A method of delaying, reducing, treating, or preventing hyperglycemia comprising administering, to a subject in need thereof, a composition comprising an effector molecule linked to an anti-MAdCAM antibody, or antigen binding fragment thereof, and a pharmaceutically acceptable carrier.
   19. The method of embodiment 18, wherein the effector molecule is a PD-1 agonist.
   20. The method of embodiment 19, wherein the PD-1 agonist is an antibody that binds to PD-1.

21. The method of any one of embodiments 18-20, where the subject has hyperglycemina, or is at risk of developing hyperglycemia.

22. The method of any one of embodiments 18-21, wherein the anti-MAdCAM antibody, or antigen binding fragment thereof, comprises:
i) a heavy chain variable region comprising heavy chain CDR1, CDR2, and CDR3 sequences, wherein the heavy chain CDR1 sequence has an amino acid sequence having at least one amino acid substitution relative to SEQ ID NO: 1499; the heavy chain CDR2 has an amino acid sequence having at least one amino acid substitution relative to SEQ ID NO: 1506; and the heavy chain CDR3 has an amino acid sequence having at least one amino acid substitution relative to SEQ ID NOs: 1507, 1531, or 1532; and
(ii) a light chain variable region comprising light chain CDR1, CDR2, and CDR3 sequences, wherein the light chain CDR1 sequence has an amino acid sequence having at least one amino acid substitution relative to SEQ ID NO: 1502; the light chain CDR2 has an amino acid sequence having at least one amino acid substitution relative to SEQ ID NO: 1497; and the light chain CDR3 has an amino acid sequence having at least one amino acid substitution relative to SEQ ID NO: 1498.
   23. The method of embodiment 22, wherein the anti-MAdCAM antibody, or antigen binding fragment thereof, comprises:
i) a heavy chain variable region comprising heavy chain CDR1, CDR2, and CDR3 sequences, wherein the heavy chain CDR1 sequence has an amino acid sequence having one or zero amino acid substitution relative to SEQ ID NO: 1499; the heavy chain CDR2 has an amino acid sequence having one or zero amino acid substitution relative to SEQ ID NO: 1506; and the heavy chain CDR3 has an amino acid sequence having one or zero amino acid substitution relative to SEQ ID NOs: 1507, 1531, or 1532, or variants of any of the foregoing; and
(ii) a light chain variable region comprising light chain CDR1, CDR2, and CDR3 sequences, wherein the light chain CDR1 sequence has an amino acid sequence having one or zero amino acid substitution relative to SEQ ID NO: 1502; the light chain CDR2 has an amino acid sequence having one or zero amino acid substitution relative to SEQ ID NO: 1497; and the light chain CDR3 has an amino acid sequence having one or zero amino acid substitution relative to SEQ ID NO: 1498, or variants of any of the foregoing.
   24. The method of embodiment 18, wherein the anti-MAdCAM antibody, or antigen binding fragment thereof, heavy chain variable region comprises a heavy chain CDR1 comprising an amino acid sequence of SEQ ID NO: 1499, a heavy chain CDR2 comprising an amino acid sequence of SEQ ID NO: 1506, and a heavy chain CDR3 comprising an amino acid sequence of SEQ ID NO: 1507.
   25. The method of embodiment 22, wherein the anti-MAdCAM antibody, or antigen binding fragment thereof, heavy chain variable region comprises a heavy chain CDR1 comprising an amino acid sequence of SEQ ID NO: 1499, a heavy chain CDR2 comprising an amino acid sequence of SEQ ID NO: 1506, and a heavy chain CDR3 comprising an amino acid sequence of SEQ ID NO: 1531.
   26. The method of embodiment 22, wherein the anti-MAdCAM antibody, or antigen binding fragment thereof, heavy chain variable region comprises a heavy chain CDR1 comprising an amino acid sequence of SEQ ID NO: 1499, a heavy chain CDR2 comprising an amino acid sequence of SEQ ID NO: 1506, and a heavy chain CDR3 comprising an amino acid sequence of SEQ ID NO: 1532.
   27. The method of any one of embodiments 22-26, wherein the anti-MAdCAM antibody, or antigen binding fragment thereof, light chain variable region comprises a light chain CDR1 comprising an amino acid sequence of SEQ ID NO: 1502, a light chain CDR2 comprising an amino acid sequence of SEQ ID NO: 1497, and a light chain CDR3 comprising an amino acid sequence of SEQ ID NO: 1498.
   28. The method of embodiment 22, wherein the anti-MAdCAM antibody, or antigen binding fragment thereof, heavy chain variable region comprises an amino acid sequence having at least 90% identity to the amino acid sequence of SEQ ID NO: 1445, 1477, or 1480 and the anti-MAdCAM antibody light chain variable region comprises an amino acid sequence having at least 90% identity to the amino acid sequence of SEQ ID NO: 1367.
   29. The method of embodiment 22, wherein the anti-MAdCAM antibody, or antigen binding fragment thereof, heavy chain variable region comprises an amino acid sequence of SEQ ID NOs: 1445, 1477, or 1480 and the anti-MAdCAM antibody light chain variable region comprises an amino acid of SEQ ID NO: 1367.
   30. The method of any one of embodiments 22-29, wherein the anti-MAdCAM antibody, or antigen binding fragment thereof, heavy chain variable region and the light chain variable region are in a Fab, or an scFv format.
   31. The method of any one of embodiments 18-30, wherein the effector molecule is an antibody, or antigen binding fragment thereof, that binds to PD-1, and wherein the antibody, or antigen binding fragment thereof, comprises:
i) a heavy chain variable region comprising heavy chain CDR1, CDR2, and CDR3 sequences, wherein the heavy chain CDR1 sequence has an amino acid sequence having at least one amino acid substitution relative to SEQ ID NO: 1481, or 1487; the heavy chain CDR2 has an amino acid sequence having at least one amino acid substitution relative to SEQ ID NO: 1482, or 1488; and the heavy chain CDR3 has an amino acid sequence having at least one amino acid substitution relative to SEQ ID NOs: 1483, or 1489; and
(ii) a light chain variable region comprising light chain CDR1, CDR2, and CDR3 sequences, wherein the light chain CDR1 sequence has an amino acid sequence having at least one amino acid substitution relative to SEQ ID NO: 1484, 1490, or 1214; the light chain CDR2 has an amino acid sequence having at least one amino acid substitution relative to SEQ ID NO: 1485, or 1491; and the light chain CDR3 has an amino acid sequence having at least one amino acid substitution relative to SEQ ID NO: 1486, or 1492.
   32. The method of any one of 19, wherein the anti-PD1 antibody comprises:
a light chain variable region comprising an amino acid sequence having at least 90% identity to an amino acid sequence selected from SEQ ID NOs: 1359, 1449, or 1479; and
a heavy chain variable region comprising an amino acid sequence having at least 90% identity to an amino acid sequence selected from SEQ ID NOs: 1438, 1446, 1447, 1448, 1476, or 1478.
   33. A method of treating Type 1 diabetes comprising administering to a subject in need thereof, a composition comprising an effector molecule linked to an antibody, or antigen binding fragment thereof, wherein:
a) the antibody is in an scFv orientation and comprises:
   i) a heavy chain variable region comprising heavy chain CDR1, CDR2, and CDR3 sequences, wherein the heavy chain CDR1 sequence has an amino acid sequence having one or zero amino acid substitution relative to SEQ ID NO: 1499; the heavy chain CDR2 has an amino acid sequence having one or zero amino acid substitution relative to SEQ ID NO: 1506; and the heavy chain CDR3 has an amino acid sequence having one or zero amino acid substitution relative to SEQ ID NOs: 1507, 1531, or 1532; and
   (ii) a light chain variable region comprising light chain CDR1, CDR2, and CDR3 sequences, wherein the light chain CDR1 sequence has an amino acid sequence having one or zero amino acid substitution relative to SEQ ID NO: 1502; the light chain CDR2 has an amino acid sequence having one or zero amino acid substitution relative to SEQ ID NO: 1497; and the light chain CDR3 has an amino acid sequence having one or zero amino acid substitution relative to SEQ ID NO: 1498; and
b) the effector molecule is in a Fab orientation and comprises:
   i) a heavy chain variable region comprising heavy chain CDR1, CDR2, and CDR3 sequences, wherein the heavy chain CDR1 sequence has an amino acid sequence having one or zero amino acid substitution relative to SEQ ID NO: 1481, or 1487; the heavy chain CDR2 has an amino acid sequence having one or zero amino acid substitution relative to SEQ ID NO: 1482, or 1488; and the heavy chain CDR3 has an amino acid sequence having one or zero amino acid substitution relative to SEQ ID NOs: 1483, or 1489; and
   (ii) a light chain variable region comprising light chain CDR1, CDR2, and CDR3 sequences, wherein the light chain CDR1 sequence has an amino acid sequence having one or zero amino acid substitution relative to SEQ ID NO: 1484, 1490, or 1214; the light chain CDR2 has an amino acid sequence having one or zero amino acid substitution relative to SEQ ID NO: 1485, or 1491; and the light chain CDR3 has an amino acid sequence having one or zero amino acid substitution relative to SEQ ID NO: 1486, or 1492.
      34. The method of embodiment 33, wherein:
a) the antibody is in an scFv orientation and comprises:
   i) a heavy chain variable region comprising heavy chain CDR1, CDR2, and CDR3 sequences, wherein the heavy chain CDR1 sequence has an amino acid sequence of SEQ ID NO: 1499; the heavy chain CDR2 has an amino acid sequence of SEQ ID NO: 1506; and the heavy chain CDR3 has an amino acid sequence of SEQ ID NOs: 1507, 1531, or 1532; and
   (ii) a light chain variable region comprising light chain CDR1, CDR2, and CDR3 sequences, wherein the light chain CDR1 sequence has an amino acid sequence of SEQ ID NO: 1502; the light chain CDR2 has an amino acid sequence of SEQ ID NO: 1497; and the light chain CDR3 has an amino acid sequence of SEQ ID NO: 1498; and
b) the effector molecule is in a Fab orientation and comprises:
   i) a heavy chain variable region comprising heavy chain CDR1, CDR2, and CDR3 sequences, wherein the heavy chain CDR1 sequence has an amino acid sequence of SEQ ID NO: 1481, or 1487; the heavy chain CDR2 has an amino acid sequence of SEQ ID NO: 1482, or 1488; and the heavy chain CDR3 has an amino acid sequence of SEQ ID NOs: 1483, or 1489; and
   (ii) a light chain variable region comprising light chain CDR1, CDR2, and CDR3 sequences, wherein the light chain CDR1 sequence has an amino acid sequence of SEQ ID NO: 1484, 1490, or 1214; the light chain CDR2 has an amino acid sequence of SEQ ID NO: 1485, or 1491; and the light chain CDR3 has an amino acid sequence of SEQ ID NO: 1486, or 1492.
      35. The method of any one of embodiments 33 or 34, wherein:
a) the antibody is in an scFv orientation and comprises:
   i) a heavy chain variable region comprising heavy chain CDR1, CDR2, and CDR3 sequences, wherein the heavy chain CDR1 sequence has the amino acid sequence of SEQ ID NO: 1499; the heavy chain CDR2 has the amino acid sequence of SEQ ID NO: 1506; and the heavy chain CDR3 has the amino acid sequence of SEQ ID NOs: 1507, or variants of any of the foregoing; and
   (ii) a light chain variable region comprising light chain CDR1, CDR2, and CDR3 sequences, wherein the light chain CDR1 sequence has the amino acid sequence of SEQ ID NO: 1502; the light chain CDR2 has the amino acid sequence of SEQ ID NO: 1497; and the light chain CDR3 has the amino acid sequence of SEQ ID NO: 1498, or variants of any of the foregoing; and
b) the effector molecule is in a Fab orientation and comprises:
   i) a heavy chain variable region comprising heavy chain CDR1, CDR2, and CDR3 sequences, wherein the heavy chain CDR1 sequence has the amino acid sequence of SEQ ID NO: 1487; the heavy chain CDR2 has the amino acid sequence of SEQ ID NO: 1488; and the heavy chain CDR3 has the amino acid sequence of SEQ ID NO: 1489, or variants of any of the foregoing; and
   (ii) a light chain variable region comprising light chain CDR1, CDR2, and CDR3 sequences, wherein the light chain CDR1 sequence has the amino acid sequence of SEQ ID NO: 1490; the light chain CDR2 has the amino acid sequence of SEQ ID NO: 1491; and the light chain CDR3 has the amino acid sequence of SEQ ID NO: 1492, or variants of any of the foregoing.
      36. The method of any one of embodiments 33 or 34, wherein:
a) the antibody is in an scFv orientation and comprises:
   i) a heavy chain variable region comprising heavy chain CDR1, CDR2, and CDR3 sequences, wherein the heavy chain CDR1 sequence has the amino acid sequence of SEQ ID NO: 1499; the heavy chain CDR2 has the amino acid sequence of SEQ ID NO: 1506; and the heavy chain CDR3 has the amino acid sequence of SEQ ID NOs: 1507, or variants of any of the foregoing; and
   (ii) a light chain variable region comprising light chain CDR1, CDR2, and CDR3 sequences, wherein the light chain CDR1 sequence has the amino acid sequence of SEQ ID NO: 1502; the light chain CDR2 has the amino acid sequence of SEQ ID NO: 1497; and the light chain CDR3 has the amino acid sequence of SEQ ID NO: 1498, or variants of any of the foregoing; and
b) the effector molecule is in a Fab orientation and comprises:
   i) a heavy chain variable region comprising heavy chain CDR1, CDR2, and CDR3 sequences, wherein the heavy chain CDR1 sequence has the amino acid sequence of SEQ ID NO: 1487; the heavy chain CDR2 has the amino acid sequence of SEQ ID NO: 1482; and the heavy chain CDR3 has the amino acid sequence of SEQ ID NO: 1483, or variants of any of the foregoing; and
   (ii) a light chain variable region comprising light chain CDR1, CDR2, and CDR3 sequences, wherein the light chain CDR1 sequence has the amino acid sequence of SEQ ID NO: 1484; the light chain CDR2 has the amino acid sequence of SEQ ID NO: 1485; and the light chain CDR3 has the amino acid sequence of SEQ ID NO: 1486, or variants of any of the foregoing.
      37. A method of treating Type 1 diabetes comprising administering to a subject in need thereof, an effector molecule linked to an antibody, or antigen binding fragment thereof, wherein:
a) the antibody is in an scFv orientation and comprises:
   a light chain variable region comprising an amino acid sequence having at least 95% identity to an amino acid sequence selected from SEQ ID NOs: 1367; and
   a heavy chain variable region comprising an amino acid sequence having at least 95% identity to an amino acid sequence selected from SEQ ID NOs: 1445; and
b) the effector molecule is in a Fab orientation and comprises:
   a light chain variable region comprising an amino acid sequence having at least 95% identity to an amino acid sequence selected from SEQ ID NOs: 1359, 1449, or 1479; and
   a heavy chain variable region comprising an amino acid sequence having at least 95% identity to an amino acid sequence selected from SEQ ID NOs: 1438, 1446, 1447, 1448, 1476, or 1478.
      38. The method of embodiment 37, wherein:
a) the antibody is in an scFv orientation and comprises:
   a light chain variable region comprising an amino acid sequence of SEQ ID NOs: 1367; and
   a heavy chain variable region comprising an amino acid sequence of SEQ ID NOs: 1445; and
b) the effector molecule is in a Fab orientation and comprises:
   a light chain variable region comprising an amino acid sequence of SEQ ID NOs: 1359, 1449, or 1479; and
   a heavy chain variable region comprising an amino acid sequence of SEQ ID NOs: 1438, 1446, 1447, 1448, 1476, or 1478.
      39. The method of any one of embodiments 37 or 38, wherein:
a) the antibody is in an scFv orientation and comprises:
   a light chain variable region comprising an amino acid sequence of SEQ ID NOs: 1367; and
   a heavy chain variable region comprising an amino acid sequence of SEQ ID NOs: 1445; and
b) the effector molecule is in a Fab orientation and comprises:
   a light chain variable region comprising an amino acid sequence of SEQ ID NOs: 1449; and
   a heavy chain variable region comprising an amino acid sequence of SEQ ID NOs: 1448.
      40. The method of any one of embodiments 37 or 38, wherein:
a) the antibody is in an scFv orientation and comprises:
   a light chain variable region comprising an amino acid sequence of SEQ ID NOs: 1367; and
   a heavy chain variable region comprising an amino acid sequence of SEQ ID NOs: 1445; and
b) the effector molecule is in a Fab orientation and comprises:
   a light chain variable region comprising an amino acid sequence of SEQ ID NOs: 1359; and
   a heavy chain variable region comprising an amino acid sequence of SEQ ID NOs: 1476.
      41. The method of any one of embodiments 37-40, wherein the heavy chain variable region and the light variable chain region are linked with a peptide linker.
      42. The method of embodiment 41, wherein the peptide linker is a glycine/serine linker.

The following examples are illustrative, but not limiting, of the compounds, compositions and methods described herein. Other suitable modifications and adaptations known to those skilled in the art are within the scope of the following embodiments.

### EXAMPLES

Non limiting examples of therapeutics, compounds, molecules, antibodies, compositions of matter, and examples may be found in PCT Application No. PCT/US2020/033707, which is hereby incorporated by reference in its entirety.

### Example 1. MAdCAM Molecule Variants With Disrupted Poly Y Patch Do Not Show Non Specific Binding to DNA and Insulin

Non-specific DNA and Insulin binding is predictive of poor pharmacokinetics (PK). An immunosorbent plate was coated with dsDNA at a concentration of 1 μg/mL or Insulin at 5 μg/mL in PBS pH 7.4, 75 μl/well, and incubated overnight at 4° C. Wells were washed with PBS pH 7.4 containing 0.05% Tween-20 (wash buffer) three times, and then blocked with 200 μl/well 1% BSA in PBS pH 7.4 (block buffer) for two hours at room temperature. After three washes with wash buffer, test antibodies (TAs) and controls Lenzilumab (humanized monoclonal antibody that binds to target colony stimulating factor 2/granulocyte-macrophage colony stimulating factor) and Elotuzumab (a humanized IgG1 monoclonal antibody that binds to SLAMF7 on NK cells) were diluted to 100 nM in PBS containing 1% BSA and 0.05% Tween-20 (assay buffer). The diluted material was added to the DNA/insulin coated plate at 75 μl/well for 1 hour at room temperature. After three washes with wash buffer, a donkey anti-human FcY HRP conjugated polyclonal antibody, diluted to 1:5000 in assay buffer, was added to the plate at 75 μl/well for 1 hr at room temperature. After three washes with wash buffer and three washes with wash buffer (with no tween-20), the assay was developed with 3,3′, 5,5′-Tetramethylbenzidine (TMB), and stopped with 1N HCL. OD 450 nm was measured. The experiment included appropriate controls for non-specific binding of test articles to the plate/block in the absence of DNA or insulin. PRNT1 showed dsDNA polyreactivity score of 45.64, and Insulin polyreactivity score of 6.21; MIAB128 showed dsDNA polyreactivity score of 33.01, and Insulin polyreactivity score of 2.62; MIAB129 showed dsDNA polyreactivity score of 3.51, and Insulin polyreactivity score of 2.43; MIAB130 showed dsDNA polyreactivity score of 29.66, and Insulin polyreactivity score of 3.26; MIAB131 showed dsDNA polyreactivity score of 13.49, and Insulin polyreactivity score of 8.00; MIAB133 showed dsDNA polyreactivity score of 44.80, and Insulin polyreactivity score of 13.16; MIAB134 showed dsDNA polyreactivity score of 45.96, and Insulin polyreactivity score of 25.53; MIAB136 showed dsDNA polyreactivity score of 51.85, and Insulin polyreactivity score of 75.37; MIAB137 showed dsDNA polyreactivity score of 43.44, and Insulin polyreactivity score of 67.33; MIAB139 showed dsDNA polyreactivity score of 1.09, and Insulin polyreactivity score of 2.08; MIAB141 showed dsDNA polyreactivity score of 33.26, and Insulin polyreactivity score of 4.18; MIAB144 showed dsDNA polyreactivity score of 47.18, and Insulin polyreactivity score of 5.07; Elotuzumab control showed dsDNA polyreactivity score of 1, and Insulin polyreactivity score of 1; and Lenzilumab control showed dsDNA polyreactivity score of 52.42, and Insulin polyreactivity score of 1.52. No non-specific binding to DNA and insulin was seen with MIAB129, MIAB139, and MIAB141. MIAB129, MIAB139, and MIAB141 were not polyreactive.

### Example 2. MAdCAM Molecule Variants with the A34N Substitution in LCDR1 do not Show Non-Specific Binding to DNA and Insulin

Non-specific DNA and Insulin binding is predictive of poor pharmacokinetics (PK). An immunosorbent plate was coated with dsDNA at a concentration of 1 μg/mL or Insulin at 5 μg/mL in PBS pH 7.4, 75 μl/well, and incubated overnight at 4° C. Wells were washed with PBS pH 7.4 containing 0.05% Tween-20 (wash buffer) three times, and then blocked with 200 μl/well 1% BSA in PBS pH 7.4 (block buffer) for two hours at room temperature. After three washes with wash buffer, TAs and controls Lenzilumab and Elotuzumab were diluted to 100 nM in PBS containing 1% BSA and 0.05% Tween-20 (assay buffer). The diluted material was added to the DNA/insulin coated plate at 75 μl/well for 1 hour at room temperature. After three washes with wash buffer, a donkey anti-human FcY HRP conjugated polyclonal antibody, diluted to 1:5000 in assay buffer, was added to the plate at 75 μl/well for 1 hr at room temperature. After three washes with wash buffer and three washes with wash buffer (with no tween-20), the assay was developed with TMB, and stopped with 1N HCL. OD 450 nm was measured. The experiment included appropriate controls for non-specific binding of test articles to the plate/block in the absence of DNA or insulin. MIAB145 showed dsDNA polyreactivity score of 43.11, and Insulin polyreactivity score of 4.58; MIAB146 showed dsDNA polyreactivity score of 24.57, and Insulin polyreactivity score of 2.61; MIAB147 showed dsDNA polyreactivity score of 8.36, and Insulin polyreactivity score of 3.81; MIAB148 showed dsDNA polyreactivity score of 3.53, and Insulin polyreactivity score of 3.63; MIAB149 showed dsDNA polyreactivity score of 27.86, and Insulin polyreactivity score of 3.53; MIAB150 showed dsDNA polyreactivity score of 9.66, and Insulin polyreactivity score of 3.74; MIAB151 showed dsDNA polyreactivity score of 2.89, and Insulin polyreactivity score of 3.63; MIAB152 showed dsDNA polyreactivity score of 7.01, and Insulin polyreactivity score of 2.83; MIAB153 showed dsDNA polyreactivity score of 1.52, and Insulin polyreactivity score of 2.46; MIAB154 showed dsDNA polyreactivity score of 8.25, and Insulin polyreactivity score of 61.91; MIAB155 showed dsDNA polyreactivity score of 1.62, and Insulin polyreactivity score of 1.99; MIAB156 showed dsDNA polyreactivity score of 4.70, and Insulin polyreactivity score of 45.25; MIAB157 showed dsDNA polyreactivity score of 6.63, and Insulin polyreactivity score of 3.99; MIAB158 showed dsDNA polyreactivity score of 1.67, and Insulin polyreactivity score of 2.67; PRNT1 showed dsDNA polyreactivity score of 38.82, and Insulin polyreactivity score of 5.02; MIAB141 showed dsDNA polyreactivity score of 1.77, and Insulin polyreactivity score of 3.60; Elotuzumab control showed dsDNA polyreactivity score of 0.95, and Insulin polyreactivity score of 1.01; and Lenzilumab control showed dsDNA polyreactivity score of 38.04, and Insulin polyreactivity score of 7.87. No non-specific binding to DNA and insulin was seen with MIAB148, MIAB151, MIAB153, MIAB155, MIAB158 and MIAB141. MIAB148, MIAB151, MIAB153, MIAB155, MIAB158 and MIAB141 were not polyreactive.

### Example 3. MAdCAM Molecule Variants With the A34N Substitution in LCDR1 Bind Human MAdCAM

Anti-human Fc biosensors were equilibrated in assay buffer (1% BSA in 1×PBS with 0.05% Tween-20) for 10 minutes before the experiment was set-up. Test articles were diluted to 5 ug/mL in assay buffer and 200 uL pipetted to 96 well plate. Human MAdCAM was titrated down, two-fold dilutions (starting at 600 nM as the highest concentration, 7-point dilution). The experiment was run using data acquisition software version 10.0 for OCTET96 RED. Test articles were captured using anti-human Fc biosensors for 180 s. Biosensors loaded with test articles were then equilibrated in assay buffer for 120 s. Association was performed in wells with huMAdCAM for 180 seconds. Dissociation was performed in wells with assay buffer for 180 s. Kinetic parameters (kon and kdis) and dissociation constant (KD) were calculated from a 1:1 global fit model using the data analysis software of the Octet96 RED software version 10. MIAB148 showed Kd (M) of 2.69E-06, Kon (1/ms) of 1.17E+05, and Kdis (1/s) of 3.14E-01; MIAB151 showed Kd of 2.96E-06, Kon of 9.87E+04, and Kdis of 2.92E-01; MIAB153 showed Kd of 8.36E-06, Kon of 6.48E+04, and Kdis of 5.43E-01; and PRNT1 showed Kd of 1.84E-08, Kon of 5.83E+05, and Kdis of 1.07E-02. MIAB148, MIAB151, and MIAB153 bound human MAdCAM with lower affinity than the parent PRNT1 molecule.

### Example 4. MAdCAM Molecule Variants With Y105I or Y105W and A34N Mutations in the VH Do Not Show Non-Specific Binding to DNA and Insulin

Non-specific DNA and Insulin binding is predictive of poor pharmacokinetics (PK). An immunosorbent plate was coated with dsDNA at a concentration of 1 μg/mL or Insulin at 5 μg/mL in PBS pH 7.4, 75 μl/well, and incubated overnight at 4° C. Wells were washed with PBS pH 7.4 containing 0.05% Tween-20 (wash buffer) three times, and then blocked with 200 μl/well 1% BSA in PBS pH 7.4 (block buffer) for two hours at room temperature. After three washes with wash buffer, TAs and controls Lenzilumab and Elotuzumab were diluted to 100 nM in PBS containing 1% BSA and 0.05% Tween-20 (assay buffer). The diluted material was added to the DNA/insulin coated plate at 75 μl/well for 1 hour at room temperature. After three washes with wash buffer, a donkey anti-human FcY HRP conjugated polyclonal antibody, diluted to 1:5000 in assay buffer, was added to the plate at 75 μl/well for 1 hr at room temperature. After three washes with wash buffer and three washes with wash buffer (with no tween-20), the assay was developed with TMB, and stopped with 1N HCL. OD 450 nm was measured. The experiment included appropriate controls for non-specific binding of test articles to the plate/block in the absence of DNA or insulin. MIAB159 showed dsDNA polyreactivity score of 9.58, and Insulin polyreactivity score of 4.66; MIAB160 showed dsDNA polyreactivity score of 42.95, and Insulin polyreactivity score of 17.80; MIAB161 showed dsDNA polyreactivity score of 25.87, and Insulin polyreactivity score of 5.00; MIAB162 showed dsDNA polyreactivity score of 21.75, and Insulin polyreactivity score of 5.31; MIAB163 showed dsDNA polyreactivity score of 28.56, and Insulin polyreactivity score of 18.53; MIAB164 showed dsDNA polyreactivity score of 25.46, and Insulin polyreactivity score of 7.07; MIAB165 showed dsDNA polyreactivity score of 19.42, and Insulin polyreactivity score of 9.53; MIAB166 showed dsDNA polyreactivity score of 37.98, and Insulin polyreactivity score of 7.89; MIAB167 showed dsDNA polyreactivity score of 26.28, and Insulin polyreactivity score of 29.56; MIAB168 showed dsDNA polyreactivity score of 7.75, and Insulin polyreactivity score of 8.35; MIAB169 showed dsDNA polyreactivity score of 3.34, and Insulin polyreactivity score of 5.59; MIAB170 showed dsDNA polyreactivity score of 2.05, and Insulin polyreactivity score of 4.73; MIAB172 showed dsDNA polyreactivity score of 26.63, and Insulin polyreactivity score of 3.79; MIAB173 showed dsDNA polyreactivity score of 29.82, and Insulin polyreactivity score of 7.10; PRNT1 showed dsDNA polyreactivity score of 34.37, and Insulin polyreactivity score of 6.91; Elotuzumab control showed dsDNA polyreactivity score of 1.05, and Insulin polyreactivity score of 1.25; and Lenzilumab control showed dsDNA polyreactivity score of 44.96, and Insulin polyreactivity score of 21.31. No non-specific binding to DNA and insulin was seen with MIAB169 and MIAB170. MIAB169 and MIAB170 were not polyreactive.

### Example 5. MAdCAM Molecule Variants With the Y105I Substitution Bind Human MAdCAM

Anti-human Fc biosensors were equilibrated in assay buffer (1% BSA in 1×PBS with 0.05% Tween-20) for 10 minutes before the experiment was set-up. Test articles were diluted to 5 ug/mL in assay buffer and 200 uL pipetted to 96 well plate. Human MAdCAM was titrated down, two-fold dilutions (starting at 600 nM as the highest concentration, 7-point dilution). Experiment was run using data acquisition software version 10.0 for OCTET96 RED. Test articles were captured using anti-human Fc biosensors for 180 s. Biosensors loaded with test articles were then equilibrated in assay buffer for 120 s. Association was performed in wells with huMAdCAM for 180 seconds. Dissociation was performed in wells with assay buffer for 180 s. Kinetic parameters (kon and kdis) and dissociation constant (KD) were calculated from a 1:1 global fit model using the data analysis software of the Octet96 RED software version 10. PRNT1 showed Kd (nM) of 26.6, Kon (1/ms) of 4.16E+05, and Kdis (1/s) of 1.11E-02; MIAB169 showed Kd of 266, Kon of 2.78E+05, and Kdis of 7.38E-02; and MIAB170 showed no binding at 1 μM human MAdCAM tested. MIAB169 bound to human MAdCAM at 10 fold lower affinity than parent PRNT1.

### Example 6. MAdCAM Molecule Variants With the Y105I Substitution Bind Human and Cyno MAdCAM

Anti-human Fc biosensors were equilibrated in assay buffer (1% BSA in 1×PBS with 0.05% Tween-20) for 10 minutes before the experiment was set-up. Test articles were diluted to 5 ug/mL in assay buffer and 200 uL pipetted to 96 well plate. Human MAdCAM was titrated down, two-fold dilutions (starting at 600 nM as the highest concentration, 7-point dilution). Experiment was run using data acquisition software version 10.0 for OCTET96 RED. Test articles were captured using anti-human Fc biosensors for 180 s. Biosensors loaded with test articles were then equilibrated in assay buffer for 120 s. Association was performed in wells with huMAdCAM for 180 seconds. Dissociation was performed in wells with assay buffer for 180 s. Kinetic parameters (kon and kdis) and dissociation constant (KD) were calculated from a 1:1 global fit model using the data analysis software of the Octet96 RED software version 10. PRNT1 showed Kd (nM) of 24 in human, Kd of 13 in cyno, and biphasic Kd in mouse; MIAB169 showed Kd of 340 in human, Kd of 153 in cyno, and biphasic Kd in mouse. MIAB169 showed lower affinity to human and cyno MAdCAM than parent PRNT1.

### Example 7. MAdCAM Molecule Variants with the Y105I Mutation do not Show Non Specific Binding to DNA and Insulin Irrespective of Expression Host

An immunosorbent plate was coated with dsDNA at a concentration of 1 μg/mL or Insulin at 5 μg/mL in PBS pH 7.4, 75 μl/well, and incubated overnight at 4° C. Wells were washed with PBS pH 7.4 containing 0.05% Tween-20 (wash buffer) three times, and then blocked with 200 μl/well 1% BSA in PBS pH 7.4 (block buffer) for two hours at room temperature. After three washes with wash buffer, TAs and controls Lenzilumab and Elotuzumab were diluted to 100 nM in PBS containing 1% BSA and 0.05% Tween-20 (assay buffer). The diluted material was added to the DNA/insulin coated plate at 75 μl/well for 1 hour at room temperature. After three washes with wash buffer, a donkey anti-human FcY HRP conjugated polyclonal antibody, diluted to 1:5000 in assay buffer, was added to the plate at 75 μl/well for 1 hr at room temperature. After three washes with wash buffer and three washes with wash buffer (with no tween-20), the assay was developed with TMB, and stopped with 1N HCL. OD 450 nm was measured. The experiment included appropriate controls for non-specific binding of test articles to the plate/block in the absence of DNA or insulin. MIAB169-CHO showed dsDNA polyreactivity score of 1.65, and Insulin polyreactivity score of 3.38; MIAB169-HEK showed dsDNA polyreactivity score of 3.36, and Insulin polyreactivity score of 6.37; Elotuzumab control showed dsDNA polyreactivity score of 1.16, and Insulin polyreactivity score of 3.43; and Lenzilumab control showed dsDNA polyreactivity score of 49.51, and Insulin polyreactivity score of 69.23. No non-specific binding to DNA and insulin was seen with MIAB169 expressed in CHO or HEK cells.

### Example 8. MAdCAM Germlined Mutants Bind Human MAdCAM

Anti-human Fc biosensors were equilibrated in assay buffer (1% BSA in 1×PBS with 0.05% Tween-20) for 10 minutes before the experiment was set-up. Test articles were diluted to 5 ug/mL in assay buffer and 200 uL pipetted to 96 well plate. Human MAdCAM was titrated down, two-fold dilutions (starting at 600 nM as the highest concentration, 7-point dilution). Experiment was run using data acquisition software version 10.0 for OCTET96 RED. Test articles were captured using anti-human Fc biosensors for 180 s. Biosensors loaded with test articles were then equilibrated in assay buffer for 120 s. Association was performed in wells with huMAdCAM for 180 seconds. Dissociation was performed in wells with assay buffer for 180 s. Kinetic parameters (kon and kdis) and dissociation constant (KD) were calculated from a 1:1 global fit model using the data analysis software of the Octet96 RED software version 10. PRNT1 showed Kd (nM) of 14, Kon (1/ms) of 6.83E+05, and Kdis (1/s) of 9.55E-03; MIAB137 (HCDR2 germlined) showed Kd (nM) of 203, Kon (1/ms) of 4.04E+05, and Kdis (1/s) of 8.2E-02; MIAB136 (HCDR1 germlined), MIAB141 (LCDR1 germlined), and MIAB141 (LCDR3 germlined) showed no binding to 150 nM human MAdCAM. MIAB137 showed a reduced binding affinity to human MAdCAM compared to parental molecule, PRNT1.

In a separate experiment PRNT1 showed Kd (nM) of 26.5, Kon (1/ms) of 4.29E+05, and Kdis (1/s) of 1.14E-02; MIAB145-001 (VK: V29I) showed Kd (nM) of 22.2, Kon (1/ms) of 4.05E+05, and Kdis (1/s) of 8.97E-03; MIAB146-001 (VK: R31S) showed Kd (nM) of 43.8, Kon (1/ms) of 4.49E+05, and Kdis (1/s) of 1.97E-02; MIAB149-001 (VK: V29I) showed Kd (nM) of 68.8, Kon (1/ms) of 3.76E+05, and Kdis (1/s) of 2.59E-02; and MIAB147-001 (VK: S32Y) showed no binding to 200 nM human MAdCAM. MIAB146 and MIAB149 have reduced binding affinity to human MAdCAM, compared to parental molecule, PRNT1.

In another experiment PRNT1 showed Kd (nM) of 21.2, Kon (1/ms) of 3.85E+05, and Kdis (1/s) of 8.16E-03; MIAB133-001 (VH: D31S) showed Kd (nM) of 20.00, Kon (1/ms) of 5.64E+05, and Kdis (1/s) of 1.13E-02; MIAB174-001 (VH: HCDR1: F32Y) showed Kd (nM) of 21.8, Kon (1/ms) of 4.45E+05, and Kdis (1/s) of 9.69E-03; MIAB175-001 (VH: HCDR1: D31S, F32Y) showed Kd (nM) of 22.6, Kon (1/ms) of 4.71E+05, and Kdis (1/s) of 1.06E-02; MIAB177-001 (VH: HCDR2: I48V, Y50A, D54S, S55G, Y57S, N59Y) showed Kd (nM) of 218, Kon (1/ms) of 3.91E+05, and Kdis (1/s) of 8.51E-02; and MIAB178-001 (VH: HCDR1: D31S, F32Y; HCDR2: Y50A, D54S, Y57S, N59Y) showed Kd (nM) of 519, Kon (1/ms) of 3.72E+05, and Kdis (1/s) of 2.20E-01. MIAB177 and MIAB178 have reduced affinity to MAdCAM compared to parental molecule, PRNT1.

In another experiment PRNT1 showed Kd (nM) of 14.8, Kon (1/ms) of 3.96E+05, and Kdis (1/s) of 5.86E-03; MIAB182-001 (HCDR1: D31S, F32Y; HCDR2: I48V, Y50A, D54S, S55G, Y57S, N59Y; VK: V29I) showed Kd (nM) of 119, Kon (1/ms) of 2.26E+05, and Kdis (1/s) of 2.67E-02; MIAB183-001 (HCDR1: D31S, F32Y; HCDR2: I48V, Y50A, D54S, S55G, Y57S, N59Y; VK: R31S) showed Kd (nM) of 362, Kon (1/ms) of 1.66E+05, and Kdis (1/s) of 5.99E-02; MIAB184-001 (HCDR1: D31S, F32T; HCDR2: I48V, Y50A, D54S, S55G, Y57S, N59Y; VK: V29I, R31S) showed Kd (nM) of 563, Kon (1/ms) of 1.45E+05, and Kdis (1/s) of 8.18E-02. Germlining heavy chain with V29I reduced MAdCAM affinity by 10-fold, germlining heavy chain with R31S reduced MAdCAM affinity by 20-fold, and germlining heavy chain and light chain reduced MAdCAM affinity by 40-fold.

### Example 9. MAdCAM-IL2 Molecules with the MAdCAM Y105I Mutation and IL-2 T3A Mutation do not Show Non-Specific Binding to DNA and Insulin Irrespective of Expression Host

An immunosorbent plate was coated with dsDNA at a concentration of 1 μg/mL or Insulin at 5 μg/mL in PBS pH 7.4, 75 μl/well, and incubated overnight at 4° C. Wells were washed with PBS pH 7.4 containing 0.05% Tween-20 (wash buffer) three times, and then blocked with 200 μl/well 1% BSA in PBS pH 7.4 (block buffer) for two hours at room temperature. After three washes with wash buffer, TAs and controls Lenzilumab and Elotuzumab were diluted to 100 nM in PBS containing 1% BSA and 0.05% Tween-20 (assay buffer). The diluted material was added to the DNA/insulin coated plate at 75 μl/well for 1 hour at room temperature. After three washes with wash buffer, a donkey anti-human FcY HRP conjugated polyclonal antibody, diluted to 1:5000 in assay buffer, was added to the plate at 75 μl/well for 1 hr at room temperature. After three washes with wash buffer and three washes with wash buffer (with no tween-20), the assay was developed with TMB, and stopped with 1N HCL. OD 450 nm was measured. The experiment included appropriate controls for non-specific binding of test articles to the plate/block in the absence of DNA or insulin. MIAB198-CHO showed dsDNA polyreactivity score of 1.36, and Insulin polyreactivity score of 3.19; MIAB198-HEK showed dsDNA polyreactivity score of 2.02, and Insulin polyreactivity score of 3.63; Elotuzumab control showed dsDNA polyreactivity score of 1.16, and Insulin polyreactivity score of 3.43; and Lenzilumab control showed dsDNA polyreactivity score of 49.51, and Insulin polyreactivity score of 69.22. No non-specific binding to DNA and insulin was seen with MIAB198 expressed in CHO or HEK cells.

### Example 10. MAdCAM-IL2 Molecules with the MAdCAM Y105I Mutation, IL-2 T3A Mutation, and Light Chain V29I Germline Mutation do not Show Non-Specific Binding to DNA and Insulin and are Expression Host Dependent

An immunosorbent plate was coated with dsDNA at a concentration of 1 μg/mL or Insulin at 5 μg/mL in PBS pH 7.4, 75 μl/well, and incubated overnight at 4° C. Wells were washed with PBS pH 7.4 containing 0.05% Tween-20 (wash buffer) three times, and then blocked with 200 μl/well 1% BSA in PBS pH 7.4 (block buffer) for two hours at room temperature. After three washes with wash buffer, TAs and controls Lenzilumab and Elotuzumab were diluted to 100 nM in PBS containing 1% BSA and 0.05% Tween-20 (assay buffer). The diluted material was added to the DNA/insulin coated plate at 75 μl/well for 1 hour at room temperature. After three washes with wash buffer, a donkey anti-human FcY HRP conjugated polyclonal antibody, diluted to 1:5000 in assay buffer, was added to the plate at 75 μl/well for 1 hr at room temperature. After three washes with wash buffer and three washes with wash buffer (with no tween-20), the assay was developed with TMB, and stopped with 1N HCL. OD 450 nm was measured. The experiment included appropriate controls for non-specific binding of test articles to the plate/block in the absence of DNA or insulin. PRNT1-CHO showed dsDNA polyreactivity score of 20.59, and Insulin polyreactivity score of 7.07; PRNT1-HEK showed dsDNA polyreactivity score of 28.08, and Insulin polyreactivity score of 13.16; MIAB185-CHO showed dsDNA polyreactivity score of 3.43, and Insulin polyreactivity score of 5.07; MIAB185-HEK showed dsDNA polyreactivity score of 23.11, and Insulin polyreactivity score of 38.37; MIAB188-CHO showed dsDNA polyreactivity score of 1.41, and Insulin polyreactivity score of 4.20; MIAB188-HEK showed dsDNA polyreactivity score of 32.80, and Insulin polyreactivity score of 83.29; Elotuzumab control showed dsDNA polyreactivity score of 0.92, and Insulin polyreactivity score of 1.09; and Lenzilumab control showed dsDNA polyreactivity score of 24.07, and Insulin polyreactivity score of 7.93. No non-specific binding to DNA and insulin was seen with MIAB185 and MIAB188 expressed in CHO cells.

### Example 11. MIAB197 is Stable for 1 Month at 4° C. and 37° C.

MIAB197 in acetate buffer was concentrated to 5 mg/mL using spin columns. Samples were collected at various concentrations and analyzed by size exclusion chromatography on an Agilent BioAdvance SEC 300 A column. MIAB197 at 5 mg/mL was incubated at 4° C. and 37° C. for up to 28 days to analyze molecule's stability over time. Samples were collected at various time points and analyzed by size exclusion chromatography on an Agilent BioAdvance SEC 300 A column. No concentration dependent aggregation was observed with MIAB197 when concentrated up to 5 mg/mL in optimized acetate buffer as seen by analytical SEC. MIAB197 at concentration of 5 mg/mL remained stable with no loss of main peak or appearance of high or low molecular weight species at 4° C. and 37° C. for 1 month.

### Example 12. MIAB197 has Favorable Thermal Stability

The PD-1-MAdCAM antibodies were submitted to the Nano DSC system (TA Instrument) for analysis. A temperature ramp of 1° C./min was performed with monitoring from 25° C. to 100° C. Thermograms of the blank buffer were subtracted from each antibody prior to analysis and the Tm values were calculated after deconvolution using the Nano DSC software. PRNT1 showed Tm (C) peak 1 of 64.5, peak 2 of 81.7, and peak 3 of 83.8; MIAB197 showed Tm peak 1 of 69.8, peak 2 of 81.7, and peak 3 of 84. MIAB197 has favorable thermal stability.

### Example 13. MIAB197 has Desirable Characteristics for Development

To characterize the identity and purity of the PD-1-MAdCAM antibodies, the antibodies were prepared in reducing labeling buffer before being submitted to the LabChip GXII system (PerkinElmer). rCE SDS revealed PRNT1 to comprise 26.5% light chain and 71.6% heavy chain; and MIAB197 to comprise 28.47% light chain and 71.53% heavy chain. MIAB197 has good characteristics for development.

### Example 14. MIAB197 has Isoelectric Point Compatible with Manufacturing

The PD-1-MAdCAM antibodies were diluted in a matrix of methyl cellulose, 4 M urea, 3-10 pharmalytes (4%), 5 mM Arginine, and pI markers (indicated below). The mixture was submitted to an iCE3 IEF Analyzer (ProteinSimple) and pre-focused at 1,500 V followed by focusing at 3,000 V. The isoelectric points of each peak were calculated from the bracketing pI markers. Capillary isoelectric focusing (cIEF) showed isoelectric peaks of 7.72 with peak area (%) of 0.60, 7.82 with peak area of 1.94, 7.96 with peak area of 5.98, 8.11 with peak area of 10.52, 8.24 with peak area of 32.43, 8.33 with peak area of 22.95, 8.39 with peak area of 12.56, 8.44 with peak area of 5.21, and 8.54 with peak area of 7.81 for PRNT1; and isoelectric peaks of 8.55 with peak area (%) of 3.63, 8.60 with peak area of 8.66, 8.69 with peak area of 18.38, 8.72 with peak area of 28.79, and 8.76 with peak area of 40.54 for MIAB197. The data demonstrate that the isoelectric peaks for MIAB197 were all above pH 8.5, with ˜70% at pI of 8.7 which is favorable for manufacturability

### Example 15. MIAB204 does not Show Non Specific Binding to DNA and Insulin

Non-specific DNA and Insulin binding is predictive of poor pharmacokinetics (PK). An immunosorbent plate was coated with dsDNA at a concentration of 1 μg/mL or Insulin at 5 μg/mL in PBS pH 7.4, 75 μl/well, and incubated overnight at 4° C. Wells were washed with PBS pH 7.4 containing 0.05% Tween-20 (wash buffer) three times, and then blocked with 200 μl/well 1% BSA in PBS pH 7.4 (block buffer) for two hours at room temperature. After three washes with wash buffer, TAs and controls Lenzilumab and Elotuzumab were diluted to 100 nM in PBS containing 1% BSA and 0.05% Tween-20 (assay buffer). The diluted material was added to the DNA/insulin coated plate at 75 μl/well for 1 hour at room temperature. After three washes with wash buffer, a donkey anti-human FcY HRP conjugated polyclonal antibody, diluted to 1:5000 in assay buffer, was added to the plate at 75 μl/well for 1 hr at room temperature. After three washes with wash buffer and three washes with wash buffer (with no tween-20), the assay was developed with TMB, and stopped with 1N HCL. OD 450 nm was measured. The experiment included appropriate controls for non-specific binding of test articles to the plate/block in the absence of DNA or insulin. MIAB204 showed dsDNA polyreactivity score of 1.66, and Insulin polyreactivity score of 8.43; Elotuzumab control showed dsDNA polyreactivity score of 1.16, and Insulin polyreactivity score of 3.43; and Lenzilumab control showed dsDNA polyreactivity score of 49.51, and Insulin polyreactivity score of 69.23. MIAB204 is not polyreactive.

### Example 16. MIAB204 has Favorable Thermal Stability

The PD-1-MAdCAM antibodies were submitted to the Nano DSC system (TA Instrument) for analysis. A temperature ramp of 1° C./min was performed with monitoring from 25° C. to 100° C. Thermograms of the blank buffer were subtracted from each antibody prior to analysis and the Tm values were calculated after deconvolution using the Nano DSC software. PRNT1 showed Tm (C) peak 1 of 64.5, peak 2 of 81.7, and peak 3 of 83.8; MIAB204 showed Tm peak 1 of 65.4, peak 2 of 69.5, and peak 3 of 84.4. MIAB204 has favorable thermal stability.

### Example 17. MIAB204 has Desirable Characteristics for Development

To characterize the identity and purity of the PD-1-MAdCAM antibodies, the antibodies were prepared in reducing labeling buffer before being submitted to the LabChip GXII system (PerkinElmer). rCE SDS revealed PRNT1 to comprise 26.5% light chain and 71.6% heavy chain; and MIAB204 to comprise one peak comprising 80.64% and second peak comprising 19.36% of the sample. MIAB204 showed different O-glycan occupancies. MIAB204 showed sufficient purity and composition identity for development.

### Example 18. MIAB204 has Isoelectric Point Compatible with Manufacturing

The sample was diluted in a matrix of methyl cellulose, 4 M urea, 3-10 pharmalytes (4%), 5 mM Arginine, and pI markers (indicated below). The mixture was submitted to an iCE3 IEF Analyzer (ProteinSimple) and pre-focused at 1,500 V followed by focusing at 3,000 V. The isoelectric points of each peak were calculated from the bracketing pI markers. Capillary isoelectric focusing (cIEF) showed isoelectric peaks of 7.72 with peak area (%) of 0.60, 7.82 with peak area of 1.94, 7.96 with peak area of 5.98, 8.11 with peak area of 10.52, 8.24 with peak area of 32.43, 8.33 with peak area of 22.95, 8.39 with peak areak of 12.56, 8.44 with peak area of 5.21, and 8.54 with peak area of 7.81 for PRNT1; and isoelectric peaks of 7.59 with peak area (%) of 2.92, 7.84 with peak area of 5.94, 8.00 with peak area of 14.88, 8.19 with peak area of 18.64, 8.29 with peak area of 5.80, 8.33 with peak area of 10.73, 8.38 with peak area of 22.13, 8.43 with peak area of 14.04, and 8.48 with peak area of 4.92 for MIAB204. Isoelectric peaks for MIAB204 show heterogeneity with most peaks having the pI greater than 8. MIAB204 is considered good for manufacturing.

### Example 19. MAdCAM-IL-2M Molecules do not Block the Interaction Between

Recombinant Human MAdCAM and alpha4beta7-positive Hut-78 T cells. 96 well plates were coated with 2.5 ug/mL recombinant human MAdCAM-Fc in PBS overnight at 4 C. Plated were blocked with DMEM containing 20% FBS for 30 minutes at 37° C., and MIAB210 (control), PRNT1, MIAB197, and a control molecule were captured for 1 hour at 37° C. in PBS. Hut-78 cells were incubated in 20% FBS DMEM supplemented with 1 mM MnCl2 for 1 hour at 37° C., and the cells were added to plates for 1 hour at 37° C. Plated were washed with PBS supplemented with 1 mM MnCl2 3 times, followed by 100 uL of cell titer glo. Plates were shaken for 2 minutes, and incubated at room temperature for another 10 minutes. Luminescence was measured and revealed lack of inhibition of MAdCAM and alpha4beta7 interaction. Optimized MAdCAM-IL-2M bi-specifics do not block MAdCAM-alpha4beta7 interaction in vitro and therefore should not interfere with the trafficking of alpha4beta7-positive T cells in vivo.

### Example 20. MAdCAM-IL-2M Molecules Selectively Induce P-STAT5 Phosphorylation on Primary Tregs Versus Teff or NK Cells when Tethered to Human/Mouse MAdCAM Expressing CHO Cells

Parental CHO cells or CHO cells over-expressing human MAdCAM or murine MAdCAM were seeded onto wells of a 96 well plate (Corning) overnight. After washing 3 times with F12+10% FBS media, the plate was blocked for 1 hour with 5 uM whole human IgG. 10 nM parental MAdCAM-IL-2M bi-specifics PRNT1 or optimized variants MIAB204 and MIAB197 were captured for 1 hour. After washing 2 times with F12+10% FBS media, freshly-isolated human PBMCs were stimulated for 60 minutes with captured IL-2MM bispecifics. Cells were then fixed for 10 minutes with BD Cytofix, permeabilized sequentially with BD Perm III, and BioLegend FOXP3 permeabilization buffer, blocked with human serum and stained for 30 minutes with antibodies against phospho-STAT5 A488, CD25 PE, FOXP3 AF647 and CD4 PerCP Cy5.5, CD3 BV421, CD56 BV785 and acquired on an Attune NXT with plate loader. PRNT1, MIAB204, and MIAB197 showed P-STAT5-positive Tregs. Accordingly, PRNT1, MIAB204, and MIAB197 selectively activate Tregs. PRNT1, MIAB204, and MIAB197 selectively induced P-STAT5 phosphorylation on primary Tregs versus Teff or NK cells when tethered to human/mouse MAdCAM expressing CHO cells.

### Example 21. V69A and Q74P Substitutions in the IL-2 Mutein are Beneficial in Improving Solubility of the Molecule

The pTT5 vectors containing the full length IgG1 heavy with C-terminally fused human IL-2 mutant and light chain encoding MIAB211 (control IgG1 mAb) were co-transfected at equimolar ratios into HEK cells. After 5-7 days, cell culture supernatants expressing MIAB211 (control IgG1 mAb) were harvested, and clarified by centrifugation and filtration through a 0.22 μm filtration device. MIAB211, was captured on Mab Select column. The column was washed with PBS pH 7.4 and the captured protein was eluted using 0.1M glycine pH 2.5, with neutralization using a tenth volume of 1M Tris pH 8.0. The protein was buffer exchanged into PBS pH 7.4, and analyzed by size exclusion chromatography on an Agilent BioAdvance SEC 300 A column. MIAB211 (control IgG1 mAb) was aggregated with only 67% monodispersed after ProA purification as shown by size exclusion chromatography. Additional polishing procedures like cation exchange improved the monodispersity to 86% which is not suitable for assays. V69A and Q74P are beneficial in improving solubility of molecule.

### Example 22. PD-1-MAdCAM Molecules With Heavy Chain Mutations Bind Human MAdCAM

Anti-human IgG Fc (AHC) biosensors were equilibrated in assay buffer for 20 minutes. Test article was diluted to 10 μg/mL in assay buffer. A seven-point two-fold serial dilution of human MAdCAM-1 was prepared in assay buffer, starting at 300 nM down to 4.69 nM. Test article was loaded on tips for 240 s followed by a 120 s association phase with MAdCAM and 120 s dissociation phase in assay buffer. Kinetic parameters (Kon and Koff) and dissociation constant (Kd) were calculated from a 1:1 global fit model using the data analysis software of the Octet96 RED software version 10. Parental molecule showed Kd (nM) of 62.8, Kon (1/ms) of 5.81E+05, and Koff (1/s) of 3.65E−02; PMAB19 showed Kd of 31.2, Kon of 5.40E+05, and Koff of 1.68E−02; PMAB20 showed Kd of 90.5, Kon of 4.11E+05, and Koff of 3.72E−02; PMAB23 showed Kd of 110, Kon of 3.55E+05, and Koff of 3.89E−02; PMAB24 showed Kd of 33.2, Kon of 4.04E+05, and Koff of 1.34E−02; PMAB25 showed Kd of 43.6, Kon of 4.86E+05, and Koff of 2.12E−02; PMAB26 showed Kd of 138, Kon of 4.76E+05, and Koff of 6.58E−02; PMAB27 showed Kd of 92.2, Kon of 138E+06, and Koff of 1.28E−01; PMAB28 showed Kd of 86.2, Kon of 1.05E+06, and Koff of 9.02E−02; and PMAB21 and PMAB22 showed no binding. PMAB19, PMAB20, PMAB23, PMAB24, PMAB25, PMAB26, PMAB27, and PMAB28 comprising heavy chain mutations bind to human MAdCAM.

### Example 23. PD-1-MAdCAM Molecules with Light Chain Mutations Bind Human MAdCAM

Anti-human IgG Fc (AHC) biosensors were equilibrated in assay buffer for 20 minutes. Test article was diluted to 10 μg/mL in assay buffer. A seven-point serial dilution of human MAdCAM-1 was prepared in assay buffer, starting at 200 nM down to 3.13 nM. Test article was loaded on tips for 240 s followed by a 120 s association phase with MAdCAM and 120 s dissociation phase in assay buffer. Kinetic parameters (Kon and Koff) and dissociation constant (Kd) were calculated from a 1:1 global fit model using the data analysis software of the Octet96 RED software version 10. Parental molecule showed Kd (nM) of 135, Kon (1/ms) of 2.52E+05, and Koff (1/s) of 3.41E−02; PMAB36 showed Kd of 109, Kon of 2.98E+05, and Koff of 3.25E-02; PMAB37 showed Kd of 285, Kon of 2.94E+05, and Koff of 8.38E−02; PMAB41 showed Kd of 43.5 uM, Kon of 2.12E+03, and Koff of 9.25E−02; PMAB42 showed Kd of 395, Kon of 2.88E+05, and Koff of 1.14E−01; and PMAB38, PMAB39, PMAB40, and PMAB43 showed no binding. PMAB36, PMAB37, PMAB41, and PMAB42 comprising light chain mutations bind to human MAdCAM.

### Example 24. PD-1-MAdCAM Molecules With Germline Mutations Bind Human MAdCAM

Anti-human IgG Fc (AHC) biosensors were equilibrated in assay buffer for 20 minutes. Test article was diluted to 10 μg/mL in assay buffer. A seven-point serial dilution of human MAdCAM-1 was prepared in assay buffer, starting at 200 nM down to 3.13 nM. Test article was loaded on tips for 240 s followed by a 120 s association phase with MAdCAM and 120 s dissociation phase in assay buffer. Kinetic parameters (Kon and Kdis) and dissociation constant (Kd) were calculated from a 1:1 global fit model using the data analysis software of the Octet96 RED software version 10. Parental molecule showed Kd (M) of 1.15E−07, Kon (1/ms) of 3.06E+05, and Kdis (1/s) of 3.51E−02; PMAB13 showed Kd of 1.32E−07, Kon of 5.42E+05, and Kdis of 7.17E−02; PMAB12 showed Kd of 6.33E−08, Kon of 7.33E+05, and Kdis of 4.64E−02; PMAB11 showed Kd of 4.66E−07, Kon of 4.19E+05, and Kdis of 1.95E−01; PMAB10 showed Kd of 1.46E−07, Kon of 6.62E+05, and Kdis of 9.67E−02; PMAB9 showed Kd of 1.59E−07, Kon of 4.55E+05, and Kdis of 7.25E−02; PMAB8 showed Kd of 7.14E−08, Kon of 6.56E+05, and Kdis of 4.69E−02; PMAB7 showed Kd of 2.36E−07, Kon of 5.76E+05, and Kdis of 1.36E−01; PMAB6 showed Kd of 1.50E−07, Kon of 6.98E+05, and Kdis of 1.05E−01; PMAB5 showed Kd of 4.13E−07, Kon of 2.90E+05, and Kdis of 1.20E−01; PMAB4 showed Kd of 4.18E−08, Kon of 1.31E+06, and Kdis of 5.47E−02; PMAB3 showed Kd of 3.33E−07, Kon of 7.17E+05, and Kdis of 2.39E−01; and PMAB2 showed Kd of 1.75E−07, Kon of 7.25E+05, and Kdis of 1.27E−01. PD-1-MAdCAM Molecules comprising germline mutations bind to human MAdCAM.

### Example 25. PD-1-MAdCAM Molecules with Single Mutations Bind Mouse MAdCAM

Anti-human IgG Fc (AHC) biosensors were equilibrated in assay buffer for 20 minutes. Test article was diluted to 10 μg/mL in assay buffer. A seven-point serial dilution of mouse MAdCAM-1 was prepared in assay buffer, starting at 500 nM down to 7.82 nM. Test article was loaded on tips for 180 s followed by a 120 s association phase with MAdCAM and 150 s dissociation phase in assay buffer. Kinetic parameters (Kon and Kdis) and dissociation constant (Kd) were calculated from a 1:1 global fit model using the data analysis software of the Octet96 RED software version 10. Parental molecule showed Kd (M) of 1.38E−07, Kon (1/ms) of 1.48E+05, Kdis (1/s) of 2.04E−02, and response of 0.1387; PMAB19 showed Kd of 1.12E−07, Kon of 1.58E+05, Kdis of 1.77E−02, and response of 0.1494; PMAB20 showed Kd of 1.18E−07, Kon of 1.63E+05, Kdis of 1.93E−02, and response of 0.1531; PMAB23 showed Kd of 1.41E−07, Kon of 1.26E+05, Kdis of 1.78E−02, and response of 0.1406; PMAB24 showed Kd of 5.24E−08, Kon of 1.14E+05, Kdis of 5.96E−03, and response of 0.0549; PMAB25 showed Kd of 1.15E−07, Kon of 1.05E+05, Kdis of 1.20E−02, and response of 0.1328; PMAB26 showed Kd of 1.34E−07, Kon of 8.79E+04, Kdis of 1.18E−02, and response of 0.132; PMAB27 showed Kd of 1.02E−06, Kon of 4.58E+03, Kdis of 4.69E−03, and response of 0.0278; PMAB28 showed Kd of 1.03E−07, Kon of 8.59E+04, Kdis of 8.86E−03, and response of 0.083; PMAB36 showed Kd of 2.06E−07, Kon of 1.22E+05, Kdis of 2.51E−02, and response of 0.1689; PMAB37 showed Kd of 1.76E−07, Kon of 1.01E+05, Kdis of 1.78E−02, and response of 0.1518; PMAB41 showed Kd of 1.19E−07, Kon of 2.08E+05, Kdis of 2.46E−02, and response of 0.1887; and PMAB42 showed Kd of 1.05E−07, Kon of 1.62E+05, Kdis of 1.70E−02, and response of 0.1287. PD-1-MAdCAM Molecules comprising single mutations bind to mouse MAdCAM.

### Example 26. PD-1-MAdCAM Molecules with Single Hydrophobic Patch Mutations Bind Human MAdCAM

Anti-human IgG Fc (AHC) biosensors were equilibrated in assay buffer for 20 minutes. Test article was diluted to 10 μg/mL in assay buffer. A seven-point serial dilution of human MAdCAM-1 was prepared in assay buffer, starting at 200 nM down to 3.13 nM. Test article was loaded on tips for 240 s followed by a 120 s association phase with MAdCAM and 120 s dissociation phase in assay buffer. Kinetic parameters (Kon and Koff) and dissociation constant (Kd) were calculated from a 1:1 global fit model using the data analysis software of the Octet96 RED software version 10. Parental molecule showed Kd (nM) of 116, Kon (1/ms) of 2.38E+05, and Koff (1/s) of 2.76E−02; PMAB45 showed Kd of 735 uM, Kon of 5.91E+02, and Koff of 4.34E−01; PMAB46 showed Kd of 37.9 uM, Kon of 8.16E+03, and Koff of 3.09E−01; PMAB47 showed Kd of 219, Kon of 3.29E+05, and Koff of 7.20E−02; PMAB48 showed Kd of 51 uM, Kon of 9.33E+03, and Koff of 1.33E−01; PMAB49 showed Kd of 142, Kon of 8.79E+04, and Koff of 1.25E−02; PMAB51 showed Kd of 93.5, Kon of 1.15E+05, and Koff of 9.52E−03; and PMAB44 and PMAB50 showed no binding. PMAB45, PMAB46, PMAB47, PMAB48, PMAB49, and PMAB51 comprising single hydrophobic patch mutations bind to human MAdCAM.

### Example 27. Optimized PD-1-MAdCAM Molecules Bind Human and Mouse MAdCAM

Kinetic parameters (Kon and Koff) and dissociation constant (Kd) were assessed and calculated as described above. Binding kinetics to human MAdCAM of the parental molecule showed Kd (M) of 3.76E−08 with a Kd error of 3.76E−08, Kon (1/ms) of 1.06E+06 with a Kon error of 3.32E+04, Kdis (1/s) of 3.98E−02 with a Kdis error of 1.36E−03, and response of 0.0839; PMAB15 showed Kd of 7.31E−08 with a Kd error of 3.82E−09, Kon of 1.15E+06 with a Kon error of 4.99E+04, Kdis of 8.39E−02 with a Kdis error of 2.42E−03, and response of 0.0655; PMAB16 showed Kd of 1.34E−07 with Kd error of 4.18E−09, Kon of 4.72E+05 with a Kon error of 1.26E+04, Kdis of 6.31E−02 with a Kdis error of 1.03E−03, and response of 0.1856; and PMAB17 showed Kd of 1.71E−08 with Kd error of 1.02E−09, Kon of 3.73E+06 with a Kon error of 1.72E+05, Kdis of 6.36E−02 with a Kdis error of 2.42E−03, and response of 0.0416. Binding kinetics to mouse MAdCAM of the parental molecule showed Kd (M) of 1.24E−07 with a Kd error of 5.96E−09, Kon (1/ms) of 3.74E+05 with a Kon error of 1.35E+04, Kdis (1/s) of 4.63E−02 with a Kdis error of 1.49E−03, and response of 0.256; PMAB15 binding was inconclusive; PMAB16 showed Kd of 3.34E−07 with Kd error of 1.34E−08, Kon of 2.48E+05 with a Kon error of 8.63E+03, Kdis of 8.28E−02 with a Kdis error of 1.64E−03, and response of 0.0407; and PMAB17 binding was inconclusive. PMAB15, PMAB16, and PMAB17 bind to human MAdCAM, and PMAB16 binds to mouse MAdCAM. While the combination of germline mutations in PMAB15 and PMAB17 have the appropriate affinity for human MAdCAM, the binding to mouse MAdCAM is severely compromised.

### Example 28. Optimized PD-1-MAdCAM Molecules Bind Human, Cyno, and Mouse MAdCAM

Kinetic parameters (Kon and Koff) and dissociation constant (Kd) were assessed and calculated as described above. Binding kinetics to human MAdCAM of PMAB57 showed Kd (M) of 1.22E−07 with a Kd error of 7.08E−09, Kon (1/ms) of 3.20E+05 with a Kon error of 1.77E+04, Kdis (1/s) of 3.89E−02 with a Kdis error of 6.84E−04, and response of 0.1804; PMAB18 showed Kd of 1.98E−07 with a Kd error of 1.23E−08, Kon of 2.59E+05 with a Kon error of 1.54E+04, Kdis of 5.11E−02 with a Kdis error of 9.93E−04, and response of 0.1842. Binding kinetics to cyno MAdCAM of PMAB57 showed Kd (M) of 4.99E−08 with a Kd error of 8.00E−10, Kon (1/ms) of 3.06E+05 with a Kon error of 4.74E+03, Kdis (1/s) of 1.53E−02 with a Kdis error of 6.39E−05, and response of 0.169; PMAB18 showed Kd of 2.26E−08 with a Kd error of 5.51E−10, Kon of 4.53E+05 with a Kon error of 1.07E+04, Kdis of 1.02E−02 with a Kdis error of 6.08E−05, and response of 0.1447. Binding kinetics to mouse MAdCAM of PMAB57 showed Kd (M) of 2.05E−07 with a Kd error of 4.09E−10, Kon (1/ms) of 2.72E+05 with a Kon error of 5.63E+03, and Kdis (1/s) of 5.58E−02 with a Kdis error of 5.43E−04; PMAB18 showed Kd of 2.01E−07 with a Kd error of 4.41E−10, Kon of 3.86E+05 with a Kon error of 1.01E+04, and Kdis of 7.76E−02 with a Kdis error of 8.45E−04. Optimized PD-1-MAdCAM antibody affinity for MAdCAM matches the targets across human, cyno, and mouse MAdCAM.

### Example 29. Optimized PD-1-MAdCAM Molecules Bind Human, Cyno, and Mouse MAdCAM Regardless of the PD-1 Agonist

Kinetic parameters (Kon and Koff) and dissociation constant (Kd) were assessed and calculated as described above. Binding kinetics to human MAdCAM of PMAB58 showed Kd of 1.35E−07, Kon of 7.12E+04, and Kdis of 9.61E−03; PMAB53 showed Kd of 4.97E−08, Kon of 1.44E+04, and Kdis of 7.16E−04; PMAB56 showed Kd of 2.08E−07, Kon of 2.36E+04, and Kdis of 4.91E−03; PMAB59 showed Kd of 1.40E−07, Kon of 3.83E+04, and Kdis of 5.37E−03; PMAB54 showed Kd of 5.92E−07, Kon of 2.36E+05, and Kdis of 1.40E−01; and PMAB55 showed Kd of 4.76E−08, Kon of 3.43E+04, and Kdis of 1.63E−03. Binding kinetics to cyno MAdCAM of PMAB58 showed Kd of 9.13E−09, Kon of 2.29E+05, and Kdis of 2.09E−03; PMAB53 showed Kd of 3.79E−07, Kon of 5.71E+04, and Kdis of 2.17E−02; PMAB56 showed Kd of 9.65E−08, Kon of 6.12E+05, and Kdis of 5.91E−02; PMAB59 showed Kd of 1.66E−08, Kon of 1.09E+05, and Kdis of 1.82E−03; PMAB54 showed Kd of 1.58E−07, Kon of 7.19E+04, and Kdis of 1.14E−02; and PMAB55 showed Kd of 4.43E−08, Kon of 2.09E+05, and Kdis 9.24E-03. Binding kinetics to mouse MAdCAM of PMAB58 showed Kd of 3.30E−07, Kon of 2.51E+05, and Kdis 8.25E−02; PMAB53 showed Kd of 1.74E−06, Kon of 1.25E+05, and Kdis of 2.17E−01; PMAB56 showed Kd of 1.61E−07, Kon of 9.12E+03, and Kdis of 1.47E−03; PMAB59 showed Kd of 1.31E−07, Kon of 1.30E+04, and Kdis of 1.70E−03; PMAB54 showed Kd of 2.48E−07, Kon of 5.57E+03, and Kdis of 1.38E−03; and PMAB55 showed Kd of 5.95E−08, Kon of 2.20E+04, and Kdis of 1.31E−03. Optimized PD-1-MAdCAM antibodies bind human, cyno, and mouse MAdCAM regardless of the PD-1 agonist, but strongly favor M to L mutants such as PMAB56 and PMAB55.

### Example 30. Optimized PD-1-MAdCAM Molecules are Thermally Stable

Thermal stability of PMAB58, PMAB53, PMAB56, PMAB59, PMAB54, and PMAB55 was evaluated as described above. The data showed that the onset of melting temperature for the M to L mutants, such as PMAB56 and PMAB55, was very similar to their respective parental clones. The M to I mutants, such as PMAB53 and PMAB54, had a higher Tm than the parental and M to L mutant, however the difference in Tm is not significant. The T aggregation onset was measured at 493 nm and produced similar values for PMAB58, PMAB53, and PMAB56; and PMAB59, PMAB54, and PMAB55. Overall, there was no significant difference in the temperature of aggregation onset. Freeze thaw stability was slightly better for the M to L mutants when compared to the initial POI, and the aSEC data showed that the initial peak heights were lower for the M to L mutants in comparison to the parental clone. Accordingly, the optimized PD-1-MAdCAM molecules are thermally stable.

### Example 31. Y105D Mutation Decreases Polyreactive Binding to Insulin

Plates were coated overnight with dsDNA and human insulin in 1×PBS. Plates were blocked with 1×PBS with 1% BSA. Antibody binding was tested at 100 nM. Sample signal was normalized to the background signal (coated wells with 2° antibody only). The data showed good dsDNA polyreactivity scores for all samples except the Y105K mutant and negative control antibody; and good Insulin polyreactivity scores for all samples except the Y105K mutant and the negative control antibody. The Y105D mutant showed improved lower Insulin polyreativity scores than other mutants. Polyreactive binding of the Y103G, Y105D, and Y105K mutants to dsDNA and human insulin shows that the Y105D hydrophobic patch mutation decreases polyreactive binding to human insulin compared to the parental antibody

### Example 32. PMAB16 has Decreased Polyreactive Binding to BVP or HEK Cell Lysate

Plates were coated with 1% Baculovirus particle (BVP) or HEK293 cell lysate (HCL) in carbonate buffer pH 9.5, 4° C. overnight. Plates were blocked with 1×PBS with 2% BSA. Antibodies were tested in triplicate for binding to BVP or HCL at 150, 50, 16.7 and 5.6 μg/mL. Signal was normalized to background signal (coated wells with 2° antibody only). BVP and HCL polyreactivity scores were lower for the PMAB16 antibody as compared to parental PMAB1 when used at 50 ug/mL or 16.7 ug/mL concentrations. Accordingly, the optimized PMAB16 antibody has decreased polyreactivity to BVP or HCL compared to the parent clone.

### Example 33. PMAB16 has Decreased Isoelectric Point

The sample was diluted in a matrix of methyl cellulose, 4 M urea, 3-10 pharmalytes (4%), 5 mM Arginine, and pI markers (indicated below). The mixture was submitted to an iCE3 IEF Analyzer (ProteinSimple) and pre-focused at 1,500 V followed by focusing at 3,000 V. The isoelectric points of each peak were calculated from the bracketing pI markers. Capillary isoelectric focusing (cIEF) showed isoelectric peaks of 8.71 with peak area (%) of 5.75, 8.97 with peak area of 19.20, 9.03 with peak area of 10.63, 9.09 with peak area of 16.92, and 9.13 with peak area of 47.50 for the PMAB1 antibody; and isoelectric peaks of 8.50 with peak area (%) of 3.90, 8.58 with peak area of 6.36, 8.73 with peak area of 45.74, and 8.76 with peak area of 44.00 for PMAB16. All isoelectric peaks for PMAB16 show the pI greater than 8. PMAB16 is considered good for manufacturing.

### Example 34. PMAB16 has Decreased Concentration Dependent Aggregation

Antibodies were affinity purified and buffer exchanged into phosphate buffer, pH 7.0 containing 8.5% sucrose and 100 mM NaCl. Each antibody was then concentrated using a centrifugal concentrator with samples taken at the indicated concentrations for analysis by analytical SEC. The optimized MAdCAM clone PMAB16 showed a decrease in concentration dependent aggregation compared to the parental PMAB1 antibody sequence.

### Example 35. PMAB16 has Good Storage Stability

Antibodies were concentrated using a centrifugal concentrator to a final concentration of 1 mg/mL. Samples were flash frozen at the indicated time points and aggregation was measured by analytical SEC. The optimized MAdCAM antibody PMAB16 showed good storage stability over 28 days at 4° C. PMAB16 stored in the accelerated stress condition of 37° C. also showed good stability out to 21 days. Accordingly, PMAB16 has good storage stability.

### Example 36. PMAB16 has Favorable Thermal Stability

The samples were submitted to the Nano DSC system (TA Instrument) for analysis. A temperature ramp of 1° C./min was performed with monitoring from 25° C. to 100° C. as described above. The data showed the Tm of PMAB16 to be lower than that of the parental molecule PMAB1, and improved storage stability at 4° C. and temperature dependent aggregation.

### Example 37. Identity of PMAB16 was Verified Via Mass Spectroscopy and CE-SDS

Sample was denatured and reduced by guanidine and DTT and deglycosylated by PNGase F before SEC separation and mass spectrometry. Mass spectroscopy showed two peaks for the PMAB16 sample, with values of 75542 Da for the peak 1 and 24258 for the peak 2, consisted with the expected mass.

Sample was prepared in reducing labeling buffer before electrophoresis using the LabChip GXII system. The data showed three peaks with fluorescence values of 26.85% for peak 1, 0.76% for peak 2, and 72.39% for peak 3, consistent with expected chain compositions for PMAB16.

### Example 38. Optimized MAdCAM Clones Retain Binding Specificity

Parental CHO cells or CHO cells expressing MAdCAM-1 were incubated with the indicated test articles. Bound test articles were detected by addition of a fluorescently conjugated anti-human IgG antibody. Optimized MAdCAM clones (PMAB18, PMAB59 and PMAB58) showed similar binding to the parental molecule (PMAB57).

### Example 39. PMAB18 Showed Improved Tethered Activity in Jurkat Assay

MAdCAM-expressing CHO cells were allowed to adhere and form a monolayer. Test articles were added at the indicated concentrations and allowed to bind for 1 h at 37° C. All wells were washed, and PD-1 reporter Jurkat cells were added. Jurkat cells were incubated with test article loaded CHO cells for 2 h at 37° C. PMAB18 showed improved tethered PD-1 agonist activity as compared to the parent antibody.

### Example 40. Optimized PD-1-MAdCAM Antibodies Co-Localize with MAdCAM-1

Fresh frozen mesenteric lymph node replicates from a 12-week BALB/c mouse were sectioned at 5 μm, fixed with acetone, blocked with blockade buffer solution for ten minutes room temperature and incubated with 1 and 10 nM titrations of test articles overnight at 4-degree Celsius. Tissues were then stained with anti-mouse MAdCAM and anti-human IgG Fc for two hours room temperature, DAPI counterstained and mounted and imaged with confocal microscopy. Clones including optimized MAdCAM (PMAB58 and PMAB18) co-localized with MAdCAM-1 expressing structures similarly to the parental clones (PMAB1 and PMAB57).

### Example 41. PMAB58 Prolongs Survival in Xenogeneic Graft Versus Host Disease

Xenogeneic graft versus host disease was induced by the transfer of human PBMC into immunodeficient mice. Beginning 10 days after cell transfer, mice were treated subcutaneously weekly with PMAB1, PMAB58, or vehicle. PMAB58 improved probability of survival to over 80 days, while the median survival time for PMAB1 was 49 days, and 41 days for vehicle. Accordingly, PMAB58 improves survival time in GVHD.

### Example 42. PMAB18 Downregulates Chemokines/Cytokines in Small Intestine

Immunocompromised NSG mice were engrafted with human PBMCs 10 days prior to treatment. Mice were treated weekly with MADCAM-PD1 bispecific (3 mg/kg) for three weeks and sacrificed. Small intestine was homogenized, normalized for total protein concentration and cytokines/chemokines were measured using the O-link proteomic platform. Data represent geometric mean and geometric standard deviation of 8 animals (log 2 scale). A student's t-test was performed on all markers; CLC4, p=0.005; IL17A, p=0.04; CXCL10, p=0.06; IFNG, p=0.03. (NPX, normalized protein expression) The vehicle data showed geoMean values of 257.9 for CCL4, 4.4 for IL17A, 14.1 for CXCL10, and 8812 for IFNG; while PMAB18 showed geoMean values of 43.8 for CCL4, 2.1 for IL17A, 3.9 for CXCL10, and 1899 for IFNG. PMAB18 reduces CCL4, IL17A, CXCL10 and IFNG in small intestine tissue from Xenogeneic graft-versus-host-disease mice. In conclusion, reduced pro-inflammatory cytokine and chemokines in target tissue suggest therapeutic effect of the MADCAM-PD1 agonist bispecific.

### Example 43. PMAB18 and PMAB58 are Detectable in Gut Tissue Through 4 Weeks Post DC Dosing

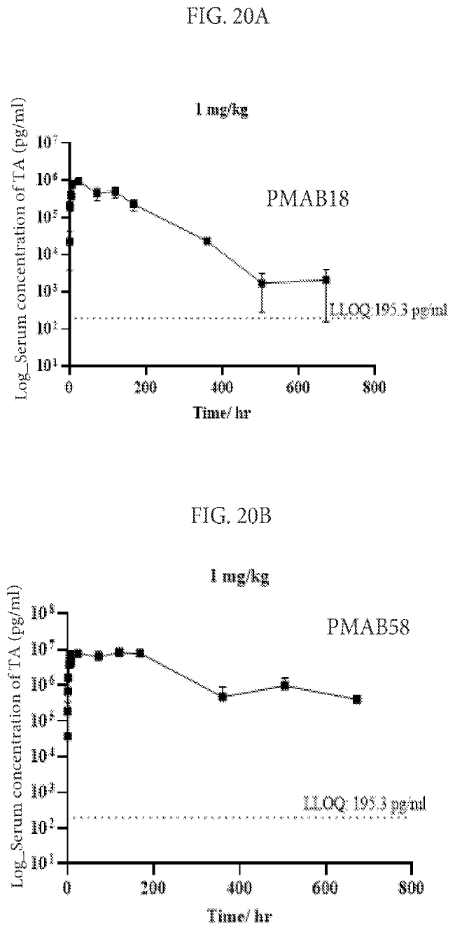

Balb/c mice were SC dosed with 1 mg/kg of PMAB18 or PMAB58. Intact PMAB18 and PMAB58 was detected in gut tissue 4 weeks after subcutaneous administration into Balb/c mice (1 mg/kg dose), revealing desirable extended PK in tissues. PMAB18 and PMAB58 remained intact and exhibited good drug like properties in systemic circulation as shown in FIGS. 20A and 20B.

### Example 44. Anti-PD-1-MAdCAM Bispecific Molecules for the Treatment of Type 1 Diabetes

Using a phage display library human/mouse/cyno cross reactive antibodies, specific for PD-1 or MAdCAM, were isolated. PD-1 antibodies were screened for their ability to antagonize or agonize the PD-1 pathway. A single triple-species cross reactive clone that was specifically agonistic with no evidence of antagonism was identified and incorporated into the final bifunctional antibody. Similarly, a triple species cross reactive non-blocking anti-MAdCAM antibody was identified using a multi-tiered screening approach. These two antibodies were combined to generate an IgG-scFv fusion with the IgG component comprising the anti-PD1 moiety comprising and the scFv moiety comprising the anti-MAdCAM moiety. NOD mice at various ages (10-16 weeks) were treated IV or SC with the anti-PD-1-MAdCAM bispecific molecule or vehicle alone. At multiple time points post treatment tissues (MLN, PLN, Pancreas) were harvested to assess for in vivo localization of test article by probing with anti-human IgG antibody specific for the F_{c} portion of the bifunctional antibody. At multiple time points post treatment cells were isolated from lymph nodes or spleen by mechanical dissociation or from the pancreas by intraductal injection of collagenase IV solution. Isolated cells were stained with a cocktail of antibodies to assess expression of the following markers; CD3, CD4, CD8, Live Dead, CD44, PD-1, Tim3 and IGRP-tet. For efficacy studies 6 week-old mice were treated once with a 500 ug bolus of anti-PD-L1 antibody (10F.9G2, an anti-PD1 antibody) and 250 ug every two days after. Test article was administered at day 0 and day 7 via sub-cutaneous injection.

Starting at 13 weeks of age bifunctional antibody could be detected concurrently with MAdCAM expressing structures in the mesenteric lymph node, pancreatic lymph node and pancreas after a single subcutaneous injection. Treatment with the anti-PD-1-MAdCAM bispecific molecule was able to specifically induce Tim3 on IGRP-tet+ cells which peaked at 4 days post treatment and was undetectable after 7 days. Additionally, the anti-PD-1-MAdCAM bispecific molecule treatment led to a significant reduction of PD-1 on bulk CD8 T cells as well as on IGRP-tet+ cells. In an accelerated PD-L1 blockade mediated model of NOD hyperglycemia simultaneous administration of the anti-PD-1-MAdCAM bispecific molecule with PD-L1 blocking antibody resulted in significantly delayed induction of hyperglycemia compared to blockade alone. Accordingly, the anti-PD-1-MAdCAM bispecific molecule modulates antigen specific and bulk CD8 T cells in pre-hyperglycemic NOD mice and is able to delay PD-L1 blockade accelerated insulitis/hyperglycemia in NOD mice.

### Example 45. Discovery of Anti-PD-1 and Anti-MAdCAM Molecules

Antibodies specific for MAdCAM-1 or PD-1 were screened for binding to human cynomolgus monkey and mouse MAdCAM-1 or PD-1. Antibodies were also tested for their ability to interfere with MAdCAM-1/α4β7 integrin binding (C) or PD-1/PD-L1 binding (D). Triple species non-blocking clones were selected for further development. Data showed multiple clones capable of binding to human cynomolgus monkey and mouse MAdCAM-1 or PD-1.

### Example 46. Discovery of PD-1 Agonist Molecules

A library of anti-PD-1 antibodies was screened for their ability to interfere with PD-L1/PD-1 signaling and their ability to activate PD-1 signaling when immobilized. Agonist antibodies that were not antagonists were selected for further development. Data showed several clones that did not interfere with endogenous PD-1/PD-L1 interactions in vitro, but activated PD-1 signaling when tethered to a surface in vitro. Molecules capable of inducing PD-1 agonist response were selected for further development.

### Example 47. Anti-PD-1-MAdCAM Bispecific Molecule Localizes to MAdCAM Expressing Structures in NOD Mice

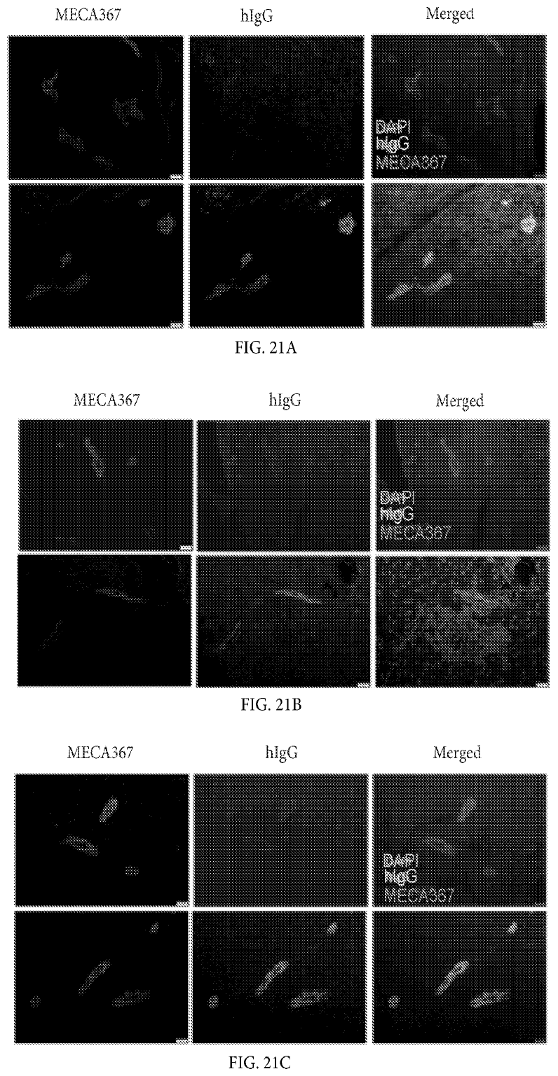

Thirteen week old female NOD mice were subcutaneously dosed with a 3 mg/kg dose of the anti-PD-1-MAdCAM bispecific molecule. Twenty four hours after treatment the mesenteric lymph node, pancreatic lymph node, and pancreas were harvested, and flash frozen in OCT. 5 uM sections were dual stained with anti-MAdCAM (MECA367, an anti-MAdCAM antibody) and anti-huIgG (to detect the anti-PD-1-MAdCAM bispecific molecule) and imaged on a confocal microscope. Data showed localization of the anti-PD-1-MAdCAM bispecific molecule to mesencetrinc lymph node, pancreatic lymph node, and pancreas (FIG. 21). Accordingly, the anti-PD-1-MAdCAM bispecific molecule localizes to MAdCAM expressing structures in NOD mice.

### Example 48. Anti-PD-1-MAdCAM Bispecific Molecule Increase Immune Activation Marker

Sixteen week old NOD mice were treated on day 0 with a SC 3 mg/kg dose of the anti-PD-1-MAdCAM bispecific molecule, untethered PD-1 agonist, or vehicle alone and sacrificed at the indicated time points. FACS analysis was run after IGRP tetramer pulldown on pool peripheral lymphoid organs. Data showed increase in % Tet+ CD44+ PD-1lo at 2 and 4 days after treatment with the anti-PD-1-MAdCAM bispecific molecule, and an increase in % IGRP+ Tim3+ at 2 and 4 days following treatment with the anti-PD-1-MAdCAM bispecific molecule. The anti-PD-1-MAdCAM bispecific molecule can regulate immune activation markers.

### Example 49. Anti-PD-1-MAdCAM Bispecific Molecule Delays PD-L1 Blockade Mediated NOD T1D

Six-week old female NOD mice were dosed IP with 500 ug on day 0, and every 2 days with 250 ug of PD-L1 blocking antibody (10F.9G2, an anti-PD1 antibody). One group of mice was also dosed with a 1 mg/kg SC dose of the anti-PD-1-MAdCAM bispecific molecule at day 0 and day 7. Blood glucose was monitored every two days by tail vein stick. Data showed delayed onset of PD-L1 blockade mediated NOD T1D after treatment with the anti-PD-1-MAdCAM bispecific molecule and PD-L1 blockade. Additionally, treatment with the anti-PD-1-MAdCAM bispecific molecule and PD-L1 blockade resulted in glucose levels that were within the levels of vehicle treated mice, and lower than mice treated with the PD-L1 blockade alone. Accordingly, the anti-PD-1-MAdCAM bispecific molecule delays PD-L1 blockade mediated NOD T1D.

The disclosures of each and every patent, patent application, and publication cited herein are hereby incorporated herein by reference in their entirety. While various embodiments have been disclosed with reference to specific aspects, it is apparent that other aspects and variations of these embodiments may be devised by others skilled in the art without departing from the true spirit and scope of the embodiments. The appended claims are intended to be construed to include all such aspects and equivalent variations.

1. A method of treating Type 1 diabetes comprising administering to a subject in need thereof, an anti-PD-1 agonist antibody linked to an anti-MAdCAM antibody, or antigen binding fragment thereof, wherein the anti-MAdCAM antibody, or antigen binding fragment thereof, comprises:
i) a heavy chain variable region comprising heavy chain CDR1, CDR2, and CDR3 sequences, wherein the heavy chain CDR1 has an amino acid sequence comprising SEQ ID NO:1499 or comprising a sequence having one amino acid substitution relative to SEQ ID NO: 1499; the heavy chain CDR2 has an amino acid sequence comprising SEQ ID NO: 1506 or comprising an amino acid sequence having one amino acid substitution relative to SEQ ID NO: 1506; and the heavy chain CDR3 has an amino acid sequence comprising SEQ ID NOs: 1507, 1531, or 1532 or comprising an amino acid sequence having one amino acid substitution relative to SEQ ID NOs: 1507, 1531, or 1532; and
(ii) a light chain variable region comprising light chain CDR1, CDR2, and CDR3 sequences, wherein the light chain CDR1 has an amino acid sequence comprising SEQ ID NO:1502 or comprising a sequence having one amino acid substitution relative to SEQ ID NO: 1502; the light chain CDR2 has an amino acid sequence comprising SEQ ID NO: 1497 or comprising an amino acid sequence having one amino acid substitution relative to SEQ ID NO: 1497; and the light chain CDR3 has an amino acid sequence comprising SEQ ID NO:1498 or comprising an amino acid sequence having one amino acid substitution relative to SEQ ID NO: 1498.

2-3. (canceled)

4. The method of claim 1, wherein the anti-MAdCAM antibody, or antigen binding fragment thereof, heavy chain variable region comprises a heavy chain CDR1 comprising the amino acid sequence of SEQ ID NO: 1499, a heavy chain CDR2 comprising the amino acid sequence of SEQ ID NO: 1506, and a heavy chain CDR3 comprising the amino acid sequence of SEQ ID NO: 1507, SEQ ID NO: 1531 or SEQ ID NO: 1532.

5-6. (canceled)

7. The method of claim 4, wherein the anti-MAdCAM antibody, or antigen binding fragment thereof, light chain variable region comprises a light chain CDR1 comprising the amino acid sequence of SEQ ID NO: 1502, a light chain CDR2 comprising the amino acid sequence of SEQ ID NO: 1497, and a light chain CDR3 comprising the amino acid sequence of SEQ ID NO: 1498.

8. The method of claim 1, wherein the anti-MAdCAM antibody, or antigen binding fragment thereof, heavy chain variable region comprises an amino acid sequence having at least 90% identity to the amino acid sequence of SEQ ID NO: 1445, 1477, or 1480 and the anti-MAdCAM antibody light chain variable region comprises an amino acid sequence having at least 90% identity to the amino acid sequence of SEQ ID NO: 1367.

9. The method of claim 8, wherein the anti-MAdCAM antibody, or antigen binding fragment thereof, heavy chain variable region comprises an amino acid sequence of SEQ ID NOs: 1445, 1477, or 1480 and the anti-MAdCAM antibody light chain variable region comprises an amino acid of SEQ ID NO: 1367.

10. The method of claim 9, wherein the anti-MAdCAM antibody, or antigen binding fragment thereof, heavy chain variable region and the light chain variable region are in a Fab, or an scFv format.

11. The method of claim 9, wherein the anti-MAdCAM antibody, or antigen binding fragment thereof, heavy chain variable region and the light variable chain region are linked with a peptide linker.

12. The method of claim 11, wherein the peptide linker is a glycine/serine linker.

13. A method of treating Type 1 diabetes comprising administering to a subject in need thereof, an effector molecule linked to an antibody, or antigen binding fragment thereof, wherein the antibody comprises:
a light chain variable region comprising an amino acid sequence having at least 90% identity to an amino acid sequence selected from SEQ ID NOs: 592, 1360, 1361, 1362, 1363, 1364, 1365, 1366, 1367, 1368, 1369, 1370, 1371, 1372, 1373, 1374, 1375, 1376, 1380, 1381, 1382, 1383, 1384, 1385, 1386, 1464, 1465, 1466, 1467, or 1543; and
a heavy chain variable region comprising an amino acid sequence having at least 90% identity to an amino acid sequence selected from SEQ ID NOs: 591, 1346, 1347, 1348, 1349, 1350, 1351, 1352, 1353, 1354, 1355, 1356, 1357, 1358, 1377, 1378, 1379, 1387, 1388, 1389, 1390, 1391, 1392, 1393, 1394, 1395, 1396, 1397, 1398, 1439, 1440, 1441, 1442, 1443, 1444, 1445, 1450, 1451, 1452, 1453, 1454, 1455, 1456, 1457, 1458, 1459, 1460, 1461, 1462, 1463, 1469, 1470, 1471, 1472, 1473, 1474, 1475, 1477, 1480, 1542, 1544, or 1545.

14. The method of claim 13, wherein the effector molecule is a PD-1 agonist.

15. The method of claim 14, wherein the PD-1 agonist is an antibody that binds to PD-1.

16. The method of claim 15, wherein the antibody that binds to PD-1 comprises:
i) a heavy chain variable region comprising heavy chain CDR1, CDR2, and CDR3 sequences, wherein the heavy chain CDR1 comprises the amino acid sequence of SEQ ID NO: 1481, or 1487; the heavy chain CDR2 comprises the amino acid sequence of SEQ ID NO: 1482, or 1488; and the heavy chain CDR3 comprises the amino acid sequence any one of SEQ ID NOs: 1483 or 1489; and
(ii) a light chain variable region comprising light chain CDR1, CDR2, and CDR3 sequences, wherein the light chain CDR1 comprises the amino acid sequence of SEQ ID NO: 1484, 1490, or 1214; the light chain CDR2 comprises the amino acid sequence of SEQ ID NO: 1485, or 1491; and the light chain CDR3 comprises the amino acid sequence any one of SEQ ID NOs: 1486 or 1492.

17. The method of claim 16, wherein the antibody that binds to PD-1 comprises:
a light chain variable region comprising an amino acid sequence having at least 90% identity to an amino acid sequence selected from SEQ ID NOs: 1359, 1449, or 1479; and
a heavy chain variable region comprising an amino acid sequence having at least 90% identity to an amino acid sequence selected from SEQ ID NOs: 1438, 1446, 1447, 1448, 1476, or 1478.

18. A method of delaying, reducing, or treating, hyperglycemia comprising administering; to a subject in need thereof; a composition comprising an effector molecule linked to an anti-MAdCAM antibody, or antigen binding fragment thereof;
and a pharmaceutically acceptable carrier.

19. The method of claim 18, wherein the effector molecule is a PD-1 agonist.

20. The method of claim 19, wherein the PD-1 agonist is an antibody that binds to PD-1.

21. (canceled)

22. The method of claim 20, wherein the anti-MAdCAM antibody, or antigen binding fragment thereof, comprises:
i) a heavy chain variable region comprising heavy chain CDR1, CDR2, and CDR3 sequences, wherein the heavy chain CDR1 comprises the amino acid sequence of SEQ ID NO: 1499; the heavy chain CDR2 comprises the amino acid of SEQ ID NO: 1506; and the heavy chain CDR3 comprises the amino acid sequence of any one of SEQ ID NOs: 1507, 1531, or 1532; and
(ii) a light chain variable region comprising light chain CDR1, CDR2, and CDR3 sequences, wherein the light chain CDR1 comprises the amino acid sequence of SEQ ID NO: 1502; the light chain CDR2 comprises the amino acid sequence of SEQ ID NO: 1497; and the light chain CDR3 comprises the amino acid sequence of SEQ ID NO: 1498.

23-42. (canceled)
